(19) United States
(12) Patent Application Publication
(10) Pub. No.: US 2025/0327045 A1
(43) Pub. Date: Oct. 23, 2025

(54) PRIME EDITOR VARIANTS, CONSTRUCTS, AND METHODS FOR ENHANCING PRIME EDITING EFFICIENCY AND PRECISION

(71) Applicants: THE BROAD INSTITUTE, INC., Cambridge, MA (US); PRESIDENT AND FELLOWS OF HARVARD COLLEGE, Cambridge, MA (US); THE TRUSTEES OF PRINCETON UNIVERSITY, Princeton, NJ (US); THE REGENTS OF THE UNIVERSITY OF CALIFORNIA, Oakland, CA (US)

(72) Inventors: David R. Liu, Cambridge, MA (US); Peter J. Chen, Cambridge, MA (US); Brittany Adamson, Princeton, NJ (US); Jeffrey Hussmann, Oakland, CA (US)

(73) Assignees: THE BROAD INSTITUTE, INC., Cambridge, MA (US); PRESIDENT AND FELLOWS OF HARVARD COLLEGE, Cambridge, MA (US); THE TRUSTEES OF PRINCETON UNIVERSITY, Princeton, NJ (US); THE REGENTS OF THE UNIVERSITY OF CALIFORNIA, Oakland, CA (US)

(21) Appl. No.: 18/271,656

(22) PCT Filed: Jan. 11, 2022
(86) PCT No.: PCT/US2022/012054

§ 371 Date: Jul. 10, 2023

Related U.S. Application Data
(60) Provisional application No. 63/136,194, filed on Jan. 11, 2021.
(60) Provisional application No. 63/176,180, filed on Apr. 16, 2021.
(60) Provisional application No. 63/176,202, filed on Apr. 16, 2021.
(60) Provisional application No. 63/194,865, filed on May 28, 2021.
(60) Provisional application No. 63/194,913, filed on May 28, 2021.
(60) Provisional application No. 63/255,897, filed on Oct. 14, 2021.

(51) Int. Cl. C12N 9/12; C12N 9/22; C12N 15/11

(52) U.S. Cl. CPC C12N 9/1276; C12N 9/226; C12N 15/111; C07K 2319/80; C12N 2310/20; C12Y 207/07049

(57) ABSTRACT

The present disclosure provides compositions and methods for prime editing with improved editing efficiency and/or reduced indel formation by inhibiting the DNA mismatch repair path way while conducting prime editing of a target site. Accordingly, the present disclosure provides a method for editing a nucleic acid molecule by prime editing that involves contacting a nucleic acid molecule with a prime editor, a pegRNA, and an inhibitor of the DNA mismatch repair pathway, thereby installing one or more modifications to the nucleic acid molecule at a target site with increased editing efficiency and/or lower indel formation. The present disclosure further provides polynucleotides for editing a DNA target site by prime editing comprising a nucleic acid sequence encoding a napDNAbp, a polymerase, and an inhibitor of the DNA mismatch repair pathway, wherein the napDNAbp and polymerase is capable in the presence of a pegRNA of installing one or more modifications in the DNA target site with increased editing efficiency and/or lower indel formation. The disclosure further provides, vectors, cells, and kits comprising the compositions and polynucleotides of the disclosure. The present disclosure also provides compositions and methods for prime editing with improved editing efficiency and/or reduced indel formation with modified prime editor fusion proteins. The disclosure further provides, vectors, cells, and kits comprising the compositions and polynucleotides of the disclosure.

### RELATED APPLICATIONS

This application is a national stage filing under 35 U.S.C. § 371 of International PCT Application PCT/US2022/012054, filed Jan. 11, 2022, which claims priority under 35 U.S.C. § 119(e) to U.S. Provisional Application, U.S. Ser. No. 63/255,897, filed on Oct. 14, 2021, to U.S. Provisional Application, U.S. Ser. No. 63/231,230, filed on Aug. 9, 2021, to U.S. Provisional Application, U.S. Ser. No. 63/194,913, filed on May 28, 2021, to U.S. Provisional Application, U.S. Ser. No. 63/194,865, filed on May 28, 2021, to U.S. Provisional Application, U.S. Ser. No. 63/176,202, filed on Apr. 16, 2021, to U.S. Provisional Application, U.S. Ser. No. 63/136,194, filed on Jan. 11, 2021, and to U.S. Provisional Application, U.S. Ser. No. 63/176,180, filed on Apr. 16, 2021, each of which is incorporated herein by reference.

### GOVERNMENT SUPPORT

This invention was made with government support under Grant Nos. AI142756, AI150551, HG009490, EB022376, EB031172, GM118062. CA072720, and GM138167 awarded by the National Institutes of Health, and Grant No. HR0011-17-2-0049 awarded by the Department of Defense. The government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jul. 8, 2023, is named B119570114US05-SEQ-TNG and is 2,594,186 bytes in size.

### INCORPORATION BY REFERENCE

In addition, this application refers to and incorporates by reference the entire contents of each of the following patent applications directed to prime editing previously filed by one or more of the present inventors: U.S. Provisional Application Ser. No. 62/820,813, filed Mar. 19, 2019; U.S. Provisional Application Ser. No. 62/858,958, filed Jun. 7, 2019; U.S. Provisional Application Ser. No. 62/889,996, filed Aug. 21, 2019; U.S. Provisional Application U.S. Ser. No. 62/922,654, filed Aug. 21, 2019; U.S. Provisional Application Ser. No. 62/913,553, filed Oct. 10, 2019; U.S. Provisional Application Ser. No. 62/973,558, filed Oct. 10, 2019; U.S. Provisional Application Ser. No. 62/931,195, filed Nov. 5, 2019; U.S. Provisional Application Ser. No. 62/944,231, filed Dec. 5, 2019: U.S. Provisional Application Ser. No. 62/974,537, filed Dec. 5, 2019; U.S. Provisional Application Ser. No. 62/991,069, filed Mar. 17, 2020; U.S. Provisional Application Ser. No. 63/100,548, filed Mar. 17, 2020; International PCT Application No. PCT/US2020/023721, filed Mar. 19, 2020; International PCT Application No. PCT/US2020/023553, filed Mar. 19, 2020; International PCT Application No. PCT/US2020/023583, filed Mar. 19, 2020; International PCT Application No. PCT/US2020/023730, filed Mar. 19, 2020; International PCT Application No. PCT/US2020/023713, filed Mar. 19, 2020; International PCT Application No. PCT/US2020/023712, filed Mar. 19, 2020; International PCT Application No. PCT/US2020/023727, filed Mar. 19, 2020; International PCT Application No. PCT/US2020/023724, filed Mar. 19, 2020; International PCT Application No. PCT/US2020/023725, filed Mar. 19, 2020; International PCT Application No. PCT/US2020/023728, filed Mar. 19, 2020; International PCT Application No. PCT/US2020/023732, filed Mar. 19, 2020; and International PCT Application No. PCT/US2020/023723, filed Mar. 19, 2020.

### BACKGROUND OF THE INVENTION

The recent development of prime editing enables the insertion, deletion, and/or replacement of genomic DNA sequences without requiring error-prone double-strand DNA breaks. See Anzalone et al., “Search-and-replace genome editing without double-strand breaks or donor DNA,” Nature, 2019, Vol. 576, pp. 149-157, the contents of which are incorporated herein by reference. Prime editing uses an engineered Cas9 nickase-reverse transcriptase fusion protein (e.g., PE1 or PE2) paired with an engineered prime editing guide RNA (pegRNA) that not only directs Cas9 to a target genomic site, but also which encodes the information for installing the desired edit. Without wishing to be bound by any particular theory, prime editing proceeds through a presumed multi-step editing process: 1) the Cas9 domain binds and nicks the target genomic DNA site, which is specified by the pegRNA's spacer sequence; 2) the reverse transcriptase domain uses the nicked genomic DNA as a primer to initiate the synthesis of an edited DNA strand using an engineered extension on the pegRNA as a template for reverse transcription—this generates a single-stranded 3′ flap containing the edited DNA sequence; 3) cellular DNA repair resolves the 3′ flap intermediate by the displacement of a 5′ flap species that occurs via invasion by the edited 3′ flap, excision of the 5′ flap containing the original DNA sequence, and ligation of the new 3′ flap to incorporate the edited DNA strand, forming a heteroduplex of one edited and one unedited strand; and 4) cellular DNA repair replaces the unedited strand within the heteroduplex using the edited strand as a template for repair, completing the editing process.

Since 2019, prime editing has been applied to introduce genetic changes in a wide variety of cells and/or organisms. Given its rapid adoption, prime editing represents a powerful tool for genomic editing. Despite its versatility and wide-scale use, prime editing efficiency can vary widely across different edit classes, target loci, and cell types (Anzalone et al., 2019). Thus, modifications to prime editing systems which result in increasing the specificity and/or efficiency of the prime editing process would significantly help advance the art. In particular, modifications that facilitate more efficient incorporation of the edited DNA strand synthesized by the prime editor into the target genomic site are desirable. It is also desirable to reduce the frequency of indel byproducts that can form as a result of prime editing. Such further modifications to prime editing would advance the art.

### SUMMARY OF THE INVENTION

In one aspect, the present disclosure relates to the observation that the efficiency and/or specificity of prime editing is impacted by a cell's own DNA mismatch repair (MMR) DNA repair pathway. MMR is a multi-factor pathway that is involved in correcting basepair mismatches and insertion/deletion mispairs generated during DNA replication and recombination. As described herein, the inventors developed a novel genetic screening method—referred to in one embodiment as “pooled CRISPRi screen for prime editing outcomes”—which led to the identification of various genetic determinates, including MMR, as affecting the efficiency and/or specificity of prime editing. Accordingly, in one aspect, the present disclosure provides novel prime editing systems comprising a means for inhibiting and/or evade the effects of MMR, thereby increasing the efficiency and/or specificity of prime editing. In one embodiment, the disclosure provides a prime editing system that comprises an MMR-inhibiting protein, such as, but not limited to, a dominant negative variant of an MMR protein, such as a dominant negative MLH1 protein (i.e., “MLH1dn”). In another embodiment, the prime editing system comprises the installation of one or more silent mutations nearby an intended edit, thereby allowing the intended edit from evading MMR recognition, even in the absence of an MMR-inhibiting protein, such as an MLH1dn. In another aspect, the disclosure provides a novel genetic screen for identifying genetic determinants, such as MMR, that impact the efficiency and/or specificity of prime editing. In still further aspects, the disclosure provides nucleic acid constructs encoding the improved prime editing systems described herein. The disclosure in other aspects also provides vectors (e.g., AAV or lentivirus vectors) comprising nucleic acids encoding the improved prime editing system described herein. In still other aspects, the disclosure provides cells comprising the improved prime editing systems described herein. The disclosure also provides in other aspects the components of the genetic screens, including nucleic acid and/or vector constructs, guide RNA, pegRNAs, cells (e.g., CRISPRi cells), and other reagents and/or materials for conducting the herein disclosed genetic screens. In still other aspects, the disclosure provides compositions and kits, e.g., pharmaceutical compositions, comprising the improved prime editing system described herein and which are capable of being administered to a cell, tissue, or organism by any suitable means, such as by gene therapy, mRNA delivery, virus-like particle delivery, or ribonucleoprotein (RNP) delivery. In yet another aspect, the present disclosure provides methods of using the improved prime editing system to install one or more edits in a target nucleic acid molecule, e.g., a genomic locus. In still another aspect, the present disclosure provides methods of treating a disease or disorder using the improved prime editing system to correct or otherwise repair one or more genetic changes (e.g., a single nucleotide polymorphism) in a target nucleic acid molecule, e.g., a genomic locus comprising one or more disease-causing mutations.

Thus, in various aspects, the present disclosure describes an improved and modified approach to prime editing that comprises inhibiting the DNA mismatch repair (MMR) system during prime editing. The inventors have surprisingly found that the editing efficiency of prime editing may be significantly increased (e.g., at least a 2-fold increase, at least a 3-fold increase, at least a 4-fold increase, at least a 5-fold increase, at least a 6-fold increase, at least a 7-fold increase, at least an 8-fold increase, at least a 9-fold increase, at least a 10-fold increase, or more) when one or more functions of the DNA mismatch repair (MMR) system are inhibited, blocked, or otherwise inactivated during prime editing (such as using the MLH1dn inhibitor of MMR). In addition, the inventors have surprisingly found that the frequency of indel formation resulting from prime editing may be significantly decreased (e.g., about a 2-fold decrease, about a 3-fold decrease, about a 4-fold decrease, about a 5-fold decrease, about a 6-fold decrease, about a 7-fold decrease, about a 8-fold decrease, about a 9-fold decrease, or about a 10-fold decrease or lower) when one or more functions of the DNA mismatch repair (MMR) system are inhibited, blocked, or otherwise inactivated during prime editing.

The present disclosure also describes in other embodiments an improved and modified approach to prime editing that comprises evading the DNA mismatch repair (MMR) system during prime editing. The inventors have surprisingly found that the editing efficiency of prime editing may be significantly increased (e.g., at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, or at least 75-fold increased) when one or more silent mutations are installed nearby a desired site for installing a genetic change by prime editing, in the presence or absence of an inhibitor of MMR. In addition, the inventors have surprisingly found that the frequency of indel formation resulting from prime editing may be significantly decreased (e.g., at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, or at least 75-fold decreased) when one or more silent mutations are installed nearby a desired site for installing a genetic change by prime editing, in the presence or absence of an inhibitor of MMR.

In some embodiments, the disclosure describes an improved prime editing system referred to herein as “PE4,” which includes PE2 plus an MLH1 dominant negative protein (e.g., wild-type MLH1 with amino acids 754-756 truncated as described further herein). In certain embodiments, the MLH1dn is expressed in trans in a cell comprising the PE2 fusion protein. The MLH1dn and the PE2 may be provided together or separate, e.g., by delivery on separate plasmids, separate vectors (e.g., AAV or lentivirus vectors), separate vector-like particles, separate ribonucleoprotein complexes (RNPs), or by delivery on the same plasmids, same vectors (e.g., AAV or lentivirus vectors), same vector-like particles, same ribonucleoprotein complexes (RNPs). In other embodiments, the MLH1dn may be fused to PE2 or otherwise associated with, coupled, or joined to PE2 such that they are co-delivered.

In other embodiments, the disclosure describes an improved prime editing system referred to as “PE5,” which includes PE3 (which is PE2 plus a second-strand nicking guide RNA) plus an MLH1 dominant negative protein (e.g., wild-type MLH1 with amino acids 754-756 truncated as described further herein). In certain embodiments, the MLH1dn is expressed in trans in a cell comprising the PE3 prime editor. The MLH1dn and the PE3 may be provide together or separate, e.g., by delivery on separate plasmids, separate vectors (e.g., AAV or lentivirus vectors), separate vector-like particles, separate ribonucleoprotein complexes (RNPs), or by delivery on the same plasmid, same vector (e.g., AAV or lentivirus vectors), same vector-like particles, same ribonucleoprotein complexes (RNPs). In other embodiments, the MLH1dn may be fused to PE3 or otherwise associated with, coupled, or joined to PE3 such that they are co-delivered.

In other aspects, the present disclosure describes an optimized PE2 prime editor architecture referred to herein as “PEmax.” PEmax is a modified form of PE2 which comprises modified reverse transcriptase codon usage. SpCas9 mutations, NLS sequences, and is described in FIG. 54B. Specifically, PEmax refers to a PE complex comprising a fusion protein comprising Cas9 (R221K N394K H840A) and a variant MMLV RT pentamutant (D200N T306K W313F T330P L603W) having the following structure: [bipartite NLS]-[Cas9(R221K)(N394K)(H840A)]-[linker]-[MMLV_RT(D200N)(T330P)(L603W)]-[bipartite NLS]-[NLS]+a desired PEgRNA, wherein the PE fusion has the amino acid sequence of SEQ ID NO: 99, which is shown as follows:

MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVD
DSFFHRLEESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPINASGVDA
KAILSARLSKSRKLENLIAQLPGEKKNGLFGNLIALSLGLTPNFKSNEDLAEDAKLQ
LSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPI
LEKMDGTEELLVKLKREDLLRKQRTFDNGSIPHQIHLGELHAILRRQEDFYPFLKD
NREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIKDK
DFLDNEENEDILEDIVLTLTLFEDREMIEERLKTYAHLFDDKVMKQLKRRRYTGW
GRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQK
GQKNSRERMKRIEEGIKELGSQILKEHPVENTQLQNEKLYLYYLQNGRDMYVDQE
LDINRLSDYDVDAIVPQSFLKDDSIDNKVLTRSDKNRGKSDNVPSEEVVKKMKNYW
RQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGT
ALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKEL
LGITIMERSSFEKNPIDFLEAKGYKEVKKDLIIKLPKYSLFELENGRKRMLASAGEL
QKGNELALPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYFDTTIDRK
RYTSTKEVLDATLIHQSITGLYETRIDLSQLGGDSGGSSGGSKRTADGSEFESPKKK
RKVSGGSSGGSTLNIEDEYRLHETSKEPDVSLGSTWLSDFPQAWAETGGMGLAVRQAP
LIIPLKATSTPVSIKQYPMSQEARLGIKPHIQRLLDQGILVPCQSPWNTPLLPVKKPGTND
YRPVQDLREVNKRVEDIHPTVPNPYNLLSGLPPSHQWYTVLDLKDAFFCLRLHPTSQPL
FAFEWRDPEMGISGQLTWTRLPQGFKNSPTLFNEALHRDLADFRIQHPDLILLQYVDDLL
LAATSELDCQQGTRALLQTLGNLGYRASAKKAQICQKQVKYLGYLLKEGQRWLTEAR
KETVMGQPTPKTPRQLREFLGKAGFCRLFIPGFAEMAAPLYPLTKPGTLFNWGPDQQKA
YQEIKQALLTAPALGLPDLTKPFELFVDEKQGYAKGVLTQKLGPWRRPVAYLSKKLDP
VAAGWPPCLRMVAAIAVLTKDAGKLTMGQPLVILAPHAVEALVKQPPDRWLSNARMT
HYQALLLDTDRVQFGPVVALNPATLLPLPEEGLQHNCLDILAEAHGTRPDLTDQPLPDA
DHTWYTDGSSLLQEGQRKAGAAVTTETEVIWAKALPAGTSAQRAELIALTQALKMAEG
KKLNVYTDSRYAFATAHIHGEIYRRRGWLTSEGKEIKNKDEILALLKALFLPKRLSIIHCP
GHQKGHSAEARGNRMADQAARKAAITETPDTSTLLIENSSPSGGSKRTADGSEFESPKK
KRKVGSGPAAKRVKLD (SEQ ID NO: 99)

KEY:
BIPARTITE SV40 NUCLEAR LOCALIZATION SEQUENCE (NLS) TOP: (SEQ ID NO: 101), BOTTOM: (SEQ ID NO: 140)
CAS9(R221K N39K H840A) (SEQ ID NO: 104)
SGGSX2-BIPARTITE SV40NLS-SGGSX2 LINKER (SEQ ID NO: 105)
M-MLV reverse transcriptase (D200N T306K W313F T330P L603W) (SEQ ID NO: 98)
Other linker sequence (SEQ ID NO: 122)
Other linker sequence (SEQ ID NO: 106)
c-Myc NLS PAAKRVKLD (SEQ ID NO: 135)

In some embodiments, the PE4 may be modified to substitute the PE2 fusion protein with PEmax. In such cases, the modified prime editing system may be referred to as “PE4max.”

In some embodiments, the PE5 may be modified to substitute the PE3 prime editor with PEmax. In such cases, the modified prime editing system may be referred to as “PE5max” and includes a second stranding nicking guide RNA.

The inventors developed prime editing which enables the insertion, deletion, and/or replacement of genomic DNA sequences without requiring error-prone double-strand DNA breaks. The present disclosure now provides an improved method of prime editing involving the blocking, inhibiting, evading, or inactivation of the MMR pathway (e.g., by inhibiting, blocking, or inactivating an MMR pathway protein, including MLH1) during prime editing, whereby doing so surprisingly results in increased editing efficiency and reduced indel formation. As used herein, “during” prime editing can embrace any suitable sequence of events, such that the prime editing step can be applied before, at the same time, or after the step of blocking, inhibiting, evading, or inactivating the MMR pathway (e.g., by targeting the inhibition of MLH1).

In various aspects and without wishing to be bound by any particular theory, prime editing uses an engineered Cas9 nickase-reverse transcriptase fusion protein (e.g., PE1 or PE2) paired with an engineered prime editing guide RNA (pegRNA) that both directs Cas9 to the target genomic site and encodes the information for installing the desired edit. Prime editing proceeds through a multi-step editing process: 1) the Cas9 domain binds and nicks the target genomic DNA site, which is specified by the pegRNA's spacer sequence; 2) the reverse transcriptase domain uses the nicked genomic DNA as a primer to initiate the synthesis of an edited DNA strand using an engineered extension on the pegRNA as a template for reverse transcription—this generates a single-stranded 3′ flap containing the edited DNA sequence; 3) cellular DNA repair resolves the 3′ flap intermediate by the displacement of a 5′ flap species that occurs via invasion by the edited 3′ flap, excision of the 5′ flap containing the original DNA sequence, and ligation of the new 3′ flap to incorporate the edited DNA strand, forming a heteroduplex of one edited and one unedited strand; and 4) cellular DNA repair replaces the unedited strand within the heteroduplex using the edited strand as a template for repair, completing the editing process.

Efficient incorporation of the desired edit requires that the newly synthesized 3′ flap contains a portion of sequence that is homologous to the genomic DNA site. This homology enables the edited 3′ flap to compete with the endogenous DNA strand (the corresponding 5′ flap) for incorporation into the DNA duplex. Because the edited 3′ flap will contain less sequence homology than the endogenous 5′ flap, the competition is expected to favor the 5′ flap strand. Thus, a potential limiting factor in the efficiency of prime editing may be the failure of the 3′ flap, which contains the edit, to effectively invade and displace the 5′ flap strand. Moreover, successful 3′ flap invasion and removal of the 5′ flap only incorporates the edit on one strand of the double-stranded DNA genome. Permanent installation of the edit requires cellular DNA repair to replace the unedited complementary DNA strand using the edited strand as a template. While the cell can be made to favor replacement of the unedited strand over the edited strand (step 4 above) by the introduction of a nick in the unedited strand adjacent to the edit using a secondary sgRNA (i.e., the PE3 system), this process still relies on a second stage of DNA repair.

This disclosure describes a modified approach to prime editing that comprises additionally inhibiting, blocking, or otherwise inactivating the DNA mismatch repair (MMR) system. In certain embodiments, the DNA mismatch repair (MMR) system can be inhibited, blocked, or otherwise inactivating one or more proteins of the MMR system, including, but not limited to MLH1, PMS2 (or MutL alpha), PMS1 (or MutL beta), MLH3 (or MutL gamma), MutS alpha (MSH2-MSH6), MutS beta (MSH2-MSH3), MSH2, MSH6, PCNA, RFC, EXO1, POLδ, and PCNA. The disclosure contemplates any suitable means by which to inhibit, block, or otherwise inactivate the DNA mismatch repair (MMR) system, including, but not limited to inactivating one or more critical proteins of the MMR system at the genetic level, e.g., by introducing one or more mutations in the genes encoding a protein of the MMR system, e.g., MLH1, PMS2 (or MutL alpha), PMS1 (or MutL beta), MLH3 (or MutL gamma), MutS alpha (MSH2-MSH6), MutS beta (MSH2-MSH3), MSH2, MSH6, PCNA, RFC, EXO1, POLδ, and PCNA.

Thus, in one aspect, the present disclosure provides a method for editing a nucleotide molecule (e.g., a genome) using prime editing while blocking, inhibiting, evading, or otherwise inactivating the DNA mismatch repair (MMR) system.

In another aspect, the present disclosure provides a method for editing a nucleotide molecule (e.g., a genome) using prime editing while blocking, inhibiting, evading, or otherwise inactivating a protein of the MMR system, e.g., MLH1, PMS2 (or MutL alpha), PMS1 (or MutL beta), MLH3 (or MutL gamma), MutS alpha (MSH2-MSH6), MutS beta (MSH2-MSH3), MSH2, MSH6, PCNA, RFC, EXO1, POLδ, and PCNA.

In still another aspect, the present disclosure provides a method for editing a nucleotide molecule (e.g., a genome) using prime editing while blocking, inhibiting, evading, or otherwise inactivating MLH1 or variant thereof.

In another aspect, the present disclosure provides a method for editing a nucleotide molecule (e.g., a genome) using prime editing while blocking, inhibiting, evading, or otherwise inactivating PMS2 (or MutL alpha) or variant thereof.

In yet another aspect, the present disclosure provides a method for editing a nucleotide molecule (e.g., a genome) using prime editing while blocking, inhibiting, evading, or otherwise inactivating PMS1 (or MutL beta) or variant thereof.

In still another aspect, the present disclosure provides a method for editing a nucleotide molecule (e.g., a genome) using prime editing while blocking, inhibiting, evading, or otherwise inactivating MLH3 (or MutL gamma) or variant thereof.

In another aspect, the present disclosure provides a method for editing a nucleotide molecule (e.g., a genome) using prime editing while blocking, inhibiting, evading, or otherwise inactivating MutS alpha (MSH2-MSH6) or variant thereof.

In still another aspect, the present disclosure provides a method for editing a nucleotide molecule (e.g., a genome) using prime editing while blocking, inhibiting, evading, or otherwise inactivating MSH2 or variant thereof.

In another aspect, the present disclosure provides a method for editing a nucleotide molecule (e.g., a genome) using prime editing while blocking, inhibiting, evading, or otherwise inactivating MSH6 or variant thereof.

In yet another aspect, the present disclosure provides a method for editing a nucleotide molecule (e.g., a genome) using prime editing while blocking, inhibiting, evading, or otherwise inactivating PCNA or variant thereof.

In still another aspect, the present disclosure provides a method for editing a nucleotide molecule (e.g., a genome) using prime editing while blocking, inhibiting, evading, or otherwise inactivating RFC or variant thereof.

In another aspect, the present disclosure provides a method for editing a nucleotide molecule (e.g., a genome) using prime editing while blocking, inhibiting, evading, or otherwise inactivating EXO1 or variant thereof.

In yet another aspect, the present disclosure provides a method for editing a nucleotide molecule (e.g., a genome) using prime editing while blocking, inhibiting, evading, or otherwise inactivating POLδ or variant thereof.

Thus, in one aspect, the present disclosure provides a method for editing a nucleotide molecule (e.g., a genome) using prime editing while blocking, inhibiting, evading, or otherwise inactivating the DNA mismatch repair (MMR) system.

In another aspect, the disclosure provides a method for evading MMR by installing one or more silent mutations nearby an intended edit, resulting in the evading of MMR and thereby improving editing efficiency of prime editing. In various embodiments, the number of silent mutations installed can be one, or two, or three, or four, or five, or six, or seven, or eight, or nine, or ten, or eleven, or twelve, or thirteen, or fourteen, or fifteen, or sixteen, or seventeen, or eighteen, or nineteen, or twenty or more. The one more silent mutations may be located upstream or downstream (or a combination if multiple silent mutations are involved) of the intended edit site, on the same or opposite strand of DNA as the intended edit site (or a combination if multiple silent mutations are involved). The silent mutations may be located upstream or downstream of the intended edit by 1, 2, 3, 4, 5, 6, 7, 8, 9, 10, 11, 12, 13, 14, 15, 16, 17, 18, 19, 20, 21, 22, 23, 24, 25 26, 27, 28, 29, 30, 31, 32, 33, 34, 35, 36, 37, 38, 39, 40, or more nucleotide positions away from the intended edit. In various embodiments, the method of evading by silent mutation installation results in a significant increase in editing efficiency of prime editing (e.g., at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, or at least 75-fold increased) when one or more silent mutations are installed nearby a desired site for installing a genetic change by prime editing, in the presence or absence of an inhibitor of MMR. In various embodiments, the method of evading MMR by silent mutation installation results in a significant decrease in the frequency of indel formation of prime editing (e.g., at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, or at least 75-fold decrease) when one or more silent mutations are installed nearby a desired site for installing a genetic change by prime editing, in the presence or absence of an inhibitor of MMR.

In another aspect, the present disclosure provides a method for editing a nucleotide molecule (e.g., a genome), comprising contacting a target nucleotide molecule with a prime editor and an inhibitor of the MMR system, e.g., an inhibitor of one or more of MLH1, PMS2 (or MutL alpha). PMS1 (or MutL beta), MLH3 (or MutL gamma), MutS alpha (MSH2-MSH6), MutS beta (MSH2-MSH3), MSH2, MSH6, PCNA, RFC, EXO1, POLδ, or PCNA. In various embodiments, the inhibitor can be a small molecule inhibitor. In other embodiments, the inhibitor can be an antibody, e.g., a neutralizing antibody. In still other embodiments, the inhibitor can be a variant of an MMR protein (e.g., a variant encoded by a dominant negative mutant of the gene encoding the MMR protein that adversely affects the function or expression of the normal wild type MMR protein, also referred to herein as a “dominant negative mutant,” “dominant negative variant,” or a “dominant negative protein,” e.g., a “dominant negative MMR protein”). In some embodiments, the inhibitor is a dominant negative variant of an MMR protein that inhibits the activity of a wild type MMR protein. For example, the inhibitor can be an MLH1 protein variant (e.g., a dominant negative mutant) of one or more of MLH1, PMS2 (or MutL alpha), PMS1 (or MutL beta), MLH3 (or MutL gamma), MutS alpha (MSH2-MSH6), MutS beta (MSH2-MSH3), MSH2, MSH6, PCNA, RFC, EXO1, POLδ, or PCNA, e.g., a dominant negative mutant of MLH1. In still other embodiments, the inhibitor can be targeted at the level of transcription, e.g., an siRNA or other nucleic acid agent that knocks down the level of a transcript encoding MLH1, PMS2 (or MutL alpha), PMS1 (or MutL beta), MLH3 (or MutL gamma), MutS alpha (MSH2-MSH6), MutS beta (MSH2-MSH3), MSH2, MSH6, PCNA, RFC, EXO1, POLδ, or PCNA. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell an mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating MLH1 or variant thereof. In another aspect, the present disclosure provides a method for editing a nucleotide molecule (e.g., a genome), comprising contacting a target nucleotide molecule with a prime editor and an inhibitor of MLH1. In various embodiments, the inhibitor can be a small molecule inhibitor. In other embodiments, the inhibitor can be an anti-MLH1 antibody, e.g., a neutralizing antibody that inactivates MLH1. In still other embodiments, the inhibitor can be a dominant negative mutant of MLH1. In still other embodiments, the inhibitor can be targeted at the level of transcription of MLH1, e.g., an siRNA or other nucleic acid agent that knocks down the level of a transcript encoding MLH1. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell an mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating PMS2 (or MutL alpha) or variant thereof. In another aspect, the present disclosure provides a method for editing a nucleotide molecule (e.g., a genome), comprising contacting a target nucleotide molecule with a prime editor and an inhibitor of PMS2 (or MutL alpha). In various embodiments, the inhibitor can be a small molecule inhibitor. In other embodiments, the inhibitor can be an anti-PMS2 (or MutL alpha) antibody, e.g., a neutralizing antibody that inactivates PMS2 (or MutL alpha). In still other embodiments, the inhibitor can be a dominant negative mutant of PMS2 (or MutL alpha). In still other embodiments, the inhibitor can be targeted at the level of transcription of PMS2 (or MutL alpha), e.g., an siRNA or other nucleic acid agent that knocks down the level of a transcript encoding ML PMS2 (or MutL alpha). In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating PMS1 (or MutL beta) or variant thereof. In another aspect, the present disclosure provides a method for editing a nucleotide molecule (e.g., a genome), comprising contacting a target nucleotide molecule with a prime editor and an inhibitor of PMS1 (or MutL beta). In various embodiments, the inhibitor can be a small molecule inhibitor. In other embodiments, the inhibitor can be an anti-PMS1 (or MutL beta) antibody, e.g., a neutralizing antibody that inactivates PMS1 (or MutL beta). In still other embodiments, the inhibitor can be a dominant negative mutant of PMS1 (or MutL beta). In still other embodiments, the inhibitor can be targeted at the level of transcription of PMS1 (or MutL beta), e.g., an siRNA or other nucleic acid agent that knocks down the level of a transcript encoding PMS1 (or MutL beta). In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating MLH3 (or MutL gamma) or variant thereof. In another aspect, the present disclosure provides a method for editing a nucleotide molecule (e.g., a genome), comprising contacting a target nucleotide molecule with a prime editor and an inhibitor of MLH3 (or MutL gamma). In various embodiments, the inhibitor can be a small molecule inhibitor. In other embodiments, the inhibitor can be an anti-MLH3 (or MutL gamma) antibody, e.g., a neutralizing antibody that inactivates MLH3 (or MutL gamma). In still other embodiments, the inhibitor can be a dominant negative mutant of MLH3 (or MutL gamma). In still other embodiments, the inhibitor can be targeted at the level of transcription of P MLH3 (or MutL gamma). e.g., an siRNA or other nucleic acid agent that knocks down the level of a transcript encoding MLH3 (or MutL gamma). In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating MutS alpha (MSH2-MSH6) or variant thereof. In another aspect, the present disclosure provides a method for editing a nucleotide molecule (e.g., a genome), comprising contacting a target nucleotide molecule with a prime editor and an inhibitor of MutS alpha (MSH2-MSH6). In various embodiments, the inhibitor can be a small molecule inhibitor. In other embodiments, the inhibitor can be an anti-MutS alpha (MSH2-MSH6) antibody, e.g., a neutralizing antibody that inactivates MutS alpha (MSH2-MSH6). In still other embodiments, the inhibitor can be a dominant negative mutant of MutS alpha (MSH2-MSH6). In still other embodiments, the inhibitor can be targeted at the level of transcription of MutS alpha (MSH2-MSH6), e.g., an siRNA or other nucleic acid agent that knocks down the level of a transcript encoding MutS alpha (MSH2-MSH6). In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating MSH2 or variant thereof. In another aspect, the present disclosure provides a method for editing a nucleotide molecule (e.g., a genome), comprising contacting a target nucleotide molecule with a prime editor and an inhibitor of MSH2. In various embodiments, the inhibitor can be a small molecule inhibitor. In other embodiments, the inhibitor can be an anti-MSH2 antibody, e.g., a neutralizing antibody that inactivates MSH2. In still other embodiments, the inhibitor can be a dominant negative mutant of MSH2. In still other embodiments, the inhibitor can be targeted at the level of transcription of MSH2, e.g., an siRNA or other nucleic acid agent that knocks down the level of a transcript encoding MSH2. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating MSH6 or variant thereof. In another aspect, the present disclosure provides a method for editing a nucleotide molecule (e.g., a genome), comprising contacting a target nucleotide molecule with a prime editor and an inhibitor of MSH6. In various embodiments, the inhibitor can be a small molecule inhibitor. In other embodiments, the inhibitor can be an anti-MSH6 antibody, e.g., a neutralizing antibody that inactivates MSH6. In still other embodiments, the inhibitor can be a dominant negative mutant of MSH6. In still other embodiments, the inhibitor can be targeted at the level of transcription of MSH6, e.g., an siRNA or other nucleic acid agent that knocks down the level of a transcript encoding MSH6. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating PCNA or variant thereof. In another aspect, the present disclosure provides a method for editing a nucleotide molecule (e.g., a genome), comprising contacting a target nucleotide molecule with a prime editor and an inhibitor of PCNA. In various embodiments, the inhibitor can be a small molecule inhibitor. In other embodiments, the inhibitor can be an anti-PCNA antibody, e.g., a neutralizing antibody that inactivates PCNA. In still other embodiments, the inhibitor can be a dominant negative mutant of PCNA. In still other embodiments, the inhibitor can be targeted at the level of transcription of PCNA, e.g., an siRNA or other nucleic acid agent that knocks down the level of a transcript encoding PCNA. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating RFC or variant thereof. In another aspect, the present disclosure provides a method for editing a nucleotide molecule (e.g., a genome), comprising contacting a target nucleotide molecule with a prime editor and an inhibitor of RFC. In various embodiments, the inhibitor can be a small molecule inhibitor. In other embodiments, the inhibitor can be an anti-RFC antibody, e.g., a neutralizing antibody that inactivates RFC. In still other embodiments, the inhibitor can be a dominant negative mutant of RFC. In still other embodiments, the inhibitor can be targeted at the level of transcription of RFC, e.g., an siRNA or other nucleic acid agent that knocks down the level of a transcript encoding RFC. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating EXO1 or variant thereof. In another aspect, the present disclosure provides a method for editing a nucleotide molecule (e.g., a genome), comprising contacting a target nucleotide molecule with a prime editor and an inhibitor of EXO1. In various embodiments, the inhibitor can be a small molecule inhibitor. In other embodiments, the inhibitor can be an anti-EXO1 antibody, e.g., a neutralizing antibody that inactivates EXO1. In still other embodiments, the inhibitor can be a dominant negative mutant of EXO1. In still other embodiments, the inhibitor can be targeted at the level of transcription of EXO1, e.g., an siRNA or other nucleic acid agent that knocks down the level of a transcript encoding EXO1. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating POLδ or variant thereof. In another aspect, the present disclosure provides a method for editing a nucleotide molecule (e.g., a genome), comprising contacting a target nucleotide molecule with a prime editor and an inhibitor of POLδ. In various embodiments, the inhibitor can be a small molecule inhibitor. In other embodiments, the inhibitor can be an anti-POLδ antibody, e.g., a neutralizing antibody that inactivates POLδ. In still other embodiments, the inhibitor can be a dominant negative mutant of POLδ. In still other embodiments, the inhibitor can be targeted at the level of transcription of POLδ, e.g., an siRNA or other nucleic acid agent that knocks down the level of a transcript encoding POLδ. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In one aspect, the present disclosure provides methods for editing a nucleic acid molecule by prime editing. In some embodiments, the method comprises contacting a nucleic acid molecule with a prime editor, a pegRNA, and an inhibitor of the DNA mismatch repair pathway, thereby installing one or more modifications to the nucleic acid molecule at a target site.

The method may increase the efficiency of prime editing and/or decrease the frequency of indel formation. In some embodiments, the prime editing efficiency is increased by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold in the presence of the inhibitor of the DNA mismatch repair pathway. In some embodiments, the frequency of indel formation is decreased by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold in the presence of the inhibitor of the DNA mismatch repair pathway.

In some embodiments, the inhibitor of the DNA mismatch repair pathway inhibits one or more proteins of the DNA mismatch repair pathway. In some embodiments, the one or more proteins is selected from the group consisting of MLH1, PMS2 (or MutL alpha), PMS1 (or MutL beta), MLH3 (or MutL gamma), MutS alpha (MSH2-MSH6), MutS beta (MSH2-MSH3), MSH2, MSH6, PCNA, RFC, EXO1, POLδ, and PCNA. In certain embodiments, the one or more proteins is MLH1. In some embodiments, MLH1 comprises an amino acid sequence of SEQ ID NO: 204, or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 204.

The inhibitor utilized in the method may be an antibody, a small molecule, a small interfering RNA (siRNA), a small non-coding microRNA, or a dominant negative variant of an MMR protein that inhibits the activity of a wild type MMR protein (e.g., a dominant negative variant of MLH1). In certain embodiments, the inhibitor is an antibody that inhibits the activity of one or more proteins of the DNA mismatch repair pathway. In some embodiments, the inhibitor is a small molecule that inhibits the activity of one or more proteins of the DNA mismatch repair pathway. In certain embodiments, the inhibitor is a small interfering RNA (siRNA) or a small non-coding microRNA that inhibits the activity of one or more proteins of the DNA mismatch repair pathway. In some embodiments, the inhibitor is a dominant negative variant of MLH1 that inhibits MLH1.

In some embodiments, the dominant negative variant is (a) MLH1 E34A (SEQ ID NO: 222), (b) MLH1 Δ756 (SEQ ID NO: 208), (c) MLH1 Δ754-756 (SEQ ID NO: 209), (d) MLH1 E34A Δ754-756 (SEQ ID NO: 210), (e) MLH1 1-335 (SEQ ID NO: 211), (f) MLH1 1-335 E34A (SEQ ID NO: 212), (g) MLH1 1-335 NLS^{SV40} (SEQ ID NO: 213), (h) MLH1 501-756 (SEQ ID NO: 215), (i) MLH1 501-753 (SEQ ID NO: 216), (j) MLH1 461-753 (SEQ ID NO: 218), or (k) NLS^{SV40} MLH1 501-753 (SEQ ID NO: 223), or a polypeptide comprising an amino acid sequence having at least 70%, at least 75%, at least 80%, at least 85%, at least 90%, at least 95%, at least 96%, at least 97%, at least 98%, at least 99%, or up to and including 100% sequence identity with any of SEQ ID NOs: 208-213, 215, 216, 218, 222, or 223.

The prime editors utilized in the methods of the present disclosure may comprise multiple components. In some embodiments, the prime editor comprises a napDNAbp and a polymerase. In some embodiments, the napDNAbp is a nuclease active Cas9 domain, a nuclease inactive Cas9 domain, or a Cas9 nickase domain or variant thereof. In certain embodiments, the napDNAbp is selected from the group consisting of: Cas9, Cas12e, Cas12d, Cas12a, Cas12b1, Cas13a, Cas12c. Cas12b2. Cas13a, Cas12c, Cas12d, Cas12e, Cas12h, Cas12i, Cas12g, Cas12f (Cas14), Cas12f1, Cas12j (Cas(b), and Argonaute and optionally has a nickase activity. In certain embodiments, the napDNAbp comprises an amino acid sequence of any one of SEQ ID NOs: 2, 4-67, or 99 (PEmax) or an amino acid sequence having at least an 80%, 85%, 90%, 95%, or 99% sequence identity with any one of SEQ ID NOs: 2, 4-67, or 99 (PEmax). In certain embodiments, the napDNAbp comprises an amino acid sequence of SEQ ID NO: 2 or SEQ ID NO: 37 (e.g., the napDNAbp of PE1 and PE2) or an amino acid sequence having at least an 80%, 85%, 90%, 95%, or 99% sequence identity with SEQ ID NO: 2. In some embodiments, the polymerase is a DNA-dependent DNA polymerase or an RNA-dependent DNA polymerase. In some embodiments, the polymerase is a reverse transcriptase. In certain embodiments, the reverse transcriptase comprises an amino acid sequence of any one of SEQ ID NOs: 69-98 or an amino acid sequence having at least an 80%, 85%, 90%, 95%, or 99% sequence identity with any one of SEQ ID NOs: 69-98.

The napDNAbp and the polymerase of the prime editor may be joined together to form a fusion protein. In some embodiments, the napDNAbp and the polymerase of the prime editor are joined by a linker to form a fusion protein. In certain embodiments, the linker comprises an amino acid sequence of any one of SEQ ID NOs: 102, or 118-131, or an amino acid sequence having at least an 80%, 85%, 90%, 95%, or 99% sequence identity with any one of SEQ ID NOs: 102, or 118-131. In some embodiments, the linker is 1, 2, 3, 4, 5, 6, 7, 8, 9, 10, 11, 12, 13, 14, 15, 16, 17, 18, 19, 20, 21, 22, 23.24, 25, 26.27, 28, 29, 30, 31.32, 33, 34.35, 36, 38, 38, 39.40, 41, 42, 43, 44, 45, 46, 47, 48, 49, or 50 amino acids in length.

The components used in the method (e.g., the prime editor, the pegRNA, and/or the inhibitor of the DNA mismatch repair pathway) may be encoded on a DNA vector. In some embodiments, the prime editor, the pegRNA, and the inhibitor of the DNA mismatch repair pathway are encoded on one or more DNA vectors. In certain embodiments, the one or more DNA vectors comprise AAV or lentivirus DNA vectors. In some embodiments, the AAV vector is serotype 1, 2, 3, 4, 5, 6, 7, 8, 9, or 10.

The prime editors utilized in the presently disclosed methods may also be further joined to additional components. In some embodiments, the prime editor as a fusion protein is further joined by a second linker to the inhibitor of the DNA mismatch repair pathway. In certain embodiments, the second linker is a self-hydrolyzing linker. In certain embodiments, the second linker comprises an amino acid sequence of any one of SEQ ID NOs: 102, 118-131, or 233-236, or an amino acid sequence having at least an 80%, 85%, 90%, 95%, or 99% sequence identity with any one of SEQ ID NOs: 102, 118-131, or 233-236. In some embodiments, the second linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In some embodiments, the one or more modifications to the nucleic acid molecule installed at the target site comprise one or more transitions, one or more transversions, one or more insertions, one or more deletions, or one more inversions. In certain embodiments, the one or more transitions are selected from the group consisting of: (a) T to C; (b) A to G; (c) C to T; and (d) G to A. In certain embodiments, the one or more transversions are selected from the group consisting of: (a) T to A; (b) T to G; (c) C to G; (d) C to A; (e) A to T; (f) A to C; (g) G to C; and (h) G to T. In certain embodiments, the one or more modifications comprises changing (1) a G:C basepair to a T:A basepair, (2) a G:C basepair to an A:T basepair, (3) a G:C basepair to a C:G basepair. (4) a T:A basepair to a G:C basepair. (5) a T:A basepair to an A:T basepair, (6) a T:A basepair to a C:G basepair, (7) a C:G basepair to a G:C basepair, (8) a C:G basepair to a T:A basepair, (9) a C:G basepair to an A:T basepair, (10) an A:T basepair to a T:A basepair. (11) an A:T basepair to a G:C basepair, or (12) an A:T basepair to a C:G basepair. In some embodiments, the one or more modifications comprises an insertion or deletion of 1, 2, 3, 4, 5, 6, 7, 8, 9, 10, 11, 12, 13, 14, 15, 16, 17, 18, 19, 20, 21, 22, 23, 24, or 25 nucleotides.

The methods of the present disclosure may be used for making corrections to one or more disease-associated genes. In some embodiments, the one or more modifications comprises a correction to a disease-associated gene. In certain embodiments, the disease-associated gene is associated with a polygenic disorder selected from the group consisting of: heart disease; high blood pressure; Alzheimer's disease; arthritis; diabetes; cancer; and obesity. In certain embodiments, the disease-associated gene is associated with a monogenic disorder selected from the group consisting of: Adenosine Deaminase (ADA) Deficiency; Alpha-1 Antitrypsin Deficiency; Cystic Fibrosis; Duchenne Muscular Dystrophy; Galactosemia; Hemochromatosis; Huntington's Disease; Maple Syrup Urine Disease; Marfan Syndrome; Neurofibromatosis Type 1; Pachyonychia Congenita; Phenylketonuria; Severe Combined Immunodeficiency; Sickle Cell Disease; Smith-Lemli-Opitz Syndrome; a trinucleotide repeat disorder; a prion disease; and Tay-Sachs Disease.

In another aspect, the present disclosure provides compositions for editing a nucleic acid molecule by prime editing. In some embodiments, the composition comprises a prime editor, a pegRNA, and an inhibitor of the DNA mismatch repair pathway, wherein the composition is capable of installing one or more modifications to the nucleic acid molecule at a target site.

The composition may increase the efficiency of prime editing and/or decrease the frequency of indel formation. In some embodiments, the prime editing efficiency is increased by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold in the presence of the inhibitor of the DNA mismatch repair pathway. In some embodiments, the frequency of indel formation is decreased by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold in the presence of the inhibitor of the DNA mismatch repair pathway.

In some embodiments, the inhibitor of the DNA mismatch repair pathway inhibits one or more proteins of the DNA mismatch repair pathway. In some embodiments, the one or more proteins is selected from the group consisting of MLH1, PMS2 (or MutL alpha), PMS1 (or MutL beta), MLH3 (or MutL gamma), MutS alpha (MSH2-MSH6), MutS beta (MSH2-MSH3), MSH2, MSH6, PCNA, RFC, EXO1, POLδ, and PCNA. In certain embodiments, the one or more proteins is MLH1. In some embodiments, MLH1 comprises an amino acid sequence of SEQ ID NO: 204, or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 204.

The inhibitor utilized in the composition may be an antibody, a small molecule, a small interfering RNA (siRNA), a small non-coding microRNA, or a dominant negative variant of an MMR protein that inhibits the activity of a wild type MMR protein (e.g., a dominant negative variant of MLH1). In certain embodiments, the inhibitor is an antibody that inhibits the activity of one or more proteins of the DNA mismatch repair pathway. In some embodiments, the inhibitor is a small molecule that inhibits the activity of one or more proteins of the DNA mismatch repair pathway. In certain embodiments, the inhibitor is a small interfering RNA (siRNA) or a small non-coding microRNA that inhibits the activity of one or more proteins of the DNA mismatch repair pathway. In some embodiments, the inhibitor is a dominant negative variant of MLH1 that inhibits MLH1.

In some embodiments, the dominant negative variant is (a) MLH1 E34A (SEQ ID NO: 222), (b) MLH1 Δ756 (SEQ ID NO: 208), (c) MLH1 Δ754-756 (SEQ ID NO: 209), (d) MLH1 E34A Δ754-756 (SEQ ID NO: 210), (e) MLH1 1-335 (SEQ ID NO: 211), (f) MLH1 1-335 E34A (SEQ ID NO: 212), (g) MLH1 1-335 NLS^{SV40} (SEQ ID NO: 213), (h) MLH1501-756 (SEQ ID NO: 215), (i) MLH1 501-753 (SEQ ID NO: 216), (j) MLH1 461-753 (SEQ ID NO: 218), or (k) NLS^{SV40} MLH1 501-753 (SEQ ID NO: 223), or a polypeptide comprising an amino acid sequence having at least 70%, at least 75%, at least 80%, at least 85%, at least 90%, at least 95%, at least 96%, at least 97%, at least 98%, at least 99%, or up to and including 100% sequence identity with any of SEQ ID NOs: 208-213, 215, 216, 218, 222, or 223.

The prime editors utilized in the compositions of the present disclosure comprise multiple components. In some embodiments, the prime editor comprises a napDNAbp and a polymerase. In some embodiments, the napDNAbp is a nuclease active Cas9 domain, a nuclease inactive Cas9 domain, or a Cas9 nickase domain or variant thereof. In certain embodiments, the napDNAbp is selected from the group consisting of: Cas9, Cas12e, Cas12d, Cas12a, Cas12b1, Cas13a, Cas12c, Cas12b2, Cas13a, Cas12c, Cas12d, Cas12e, Cas12h, Cas12i, Cas12g, Cas12f (Cas14), Cas12f1, Cas12j (Cas(D), and Argonaute and optionally has a nickase activity. In certain embodiments, the napDNAbp comprises an amino acid sequence of any one of SEQ ID NOs: 2, 4-67, or 99 (PEmax) or an amino acid sequence having at least an 80%, 85%, 90%, 95%, or 99% sequence identity with any one of SEQ ID NOs: 2, 4-67, or 99 (PEmax). In certain embodiments, the napDNAbp comprises an amino acid sequence of SEQ ID NO: 2 or SEQ ID NO: 37 (i.e., the napDNAbp of PE1 and PE2) or an amino acid sequence having at least an 80%, 85%, 90%, 95%, or 99% sequence identity with SEQ ID NO: 2 or SEQ ID NO: 37. In some embodiments, the polymerase is a DNA-dependent DNA polymerase or an RNA-dependent DNA polymerase. In some embodiments, the polymerase is a reverse transcriptase. In certain embodiments, the reverse transcriptase comprises an amino acid sequence of any one of SEQ ID NOs: 69-98 or an amino acid sequence having at least an 80%, 85%, 90%, 95%, or 99% sequence identity with any one of SEQ ID NOs: 69-98.

The napDNAbp and the polymerase of the prime editor may be joined together to form a fusion protein. In some embodiments, the napDNAbp and the polymerase of the prime editor are joined by a linker to form a fusion protein. In certain embodiments, the linker comprises an amino acid sequence of any one of SEQ ID NOs: 102, 118-131, or an amino acid sequence having at least an 80%, 85%, 90%, 95%, or 99% sequence identity with any one of SEQ ID NOs: 102, 118-131. In some embodiments, the linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 38, 38, 39, 40, 41, 42, 43, 44, 45, 46, 47, 48, 49, or 50 amino acids in length.

The components used in the compositions disclosed herein (e.g., the prime editor, the pegRNA, and/or the inhibitor of the DNA mismatch repair pathway) may be encoded on a DNA vector. In some embodiments, the prime editor, the pegRNA, and the inhibitor of the DNA mismatch repair pathway are encoded on one or more DNA vectors. In certain embodiments, the one or more DNA vectors comprise AAV or lentivirus DNA vectors. In some embodiments, the AAV vector is serotype 1, 2, 3, 4, 5, 6, 7, 8, 9, or 10.

The prime editors utilized in the presently disclosed compositions may also be further joined to additional components. In some embodiments, the prime editor as a fusion protein is further joined by a second linker to the inhibitor of the DNA mismatch repair pathway. In certain embodiments, the second linker is a self-hydrolyzing linker. In certain embodiments, the second linker comprises an amino acid sequence of any one of SEQ ID NOs: 102, 118-131, or 233-236, or an amino acid sequence having at least an 80%, 85%, 90%, 95%, or 99% sequence identity with any one of SEQ ID NOs: 102, 118-131, or 233-236. In some embodiments, the second linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In some embodiments, the one or more modifications to the nucleic acid molecule installed at the target site comprise one or more transitions, one or more transversions, one or more insertions, one or more deletions, or one more inversions. In certain embodiments, the one or more transitions are selected from the group consisting of: (a) T to C; (b) A to G; (c) C to T; and (d) G to A. In certain embodiments, the one or more transversions are selected from the group consisting of: (a) T to A; (b) T to G; (c) C to G; (d) C to A; (e) A to T; (f) A to C; (g) G to C; and (h) G to T. In certain embodiments, the one or more modifications comprises changing (1) a G:C basepair to a T:A basepair, (2) a G:C basepair to an A:T basepair, (3) a G:C basepair to a C:G basepair, (4) a T:A basepair to a G:C basepair, (5) a T:A basepair to an A:T basepair, (6) a T:A basepair to a C:G basepair, (7) a C:G basepair to a G:C basepair, (8) a C:G basepair to a T:A basepair, (9) a C:G basepair to an A:T basepair, (10) an A:T basepair to a T:A basepair, (11) an A:T basepair to a G:C basepair, or (12) an A:T basepair to a C:G basepair. In some embodiments, the one or more modifications comprises an insertion or deletion of 1, 2, 3, 4, 5, 6, 7, 8, 9, 10, 11, 12, 13, 14, 15, 16, 17, 18, 19, 20, 21, 22, 23, 24, or 25 nucleotides.

The compositions of the present disclosure may be used for making corrections to one or more disease-associated genes. In some embodiments, the one or more modifications comprises a correction to a disease-associated gene. In certain embodiments, the disease-associated gene is associated with a polygenic disorder selected from the group consisting of: heart disease; high blood pressure; Alzheimer's disease; arthritis; diabetes; cancer; and obesity. In certain embodiments, the disease-associated gene is associated with a monogenic disorder selected from the group consisting of: Adenosine Deaminase (ADA) Deficiency; Alpha-1 Antitrypsin Deficiency; Cystic Fibrosis; Duchenne Muscular Dystrophy; Galactosemia; Hemochromatosis; Huntington's Disease; Maple Syrup Urine Disease; Marfan Syndrome; Neurofibromatosis Type 1; Pachyonychia Congenita; Phenylketonuria; Severe Combined Immunodeficiency; Sickle Cell Disease; Smith-Lemli-Opitz Syndrome; a trinucleotide repeat disorder; a prion disease; and Tay-Sachs Disease.

In another aspect, this disclosure provides polynucleotides for editing a DNA target site by prime editing. In some embodiments, the polynucleotide comprises a nucleic acid sequence encoding a napDNAbp, a polymerase, and an inhibitor of the DNA mismatch repair pathway, wherein the napDNAbp and polymerase is capable in the presence of a pegRNA of installing one or more modifications in the DNA target site.

The polynucleotide may increase the efficiency of prime editing and/or decrease the frequency of indel formation. In some embodiments, the prime editing efficiency is increased by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold in the presence of the inhibitor of the DNA mismatch repair pathway. In some embodiments, the frequency of indel formation is decreased by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold in the presence of the inhibitor of the DNA mismatch repair pathway.

In some embodiments, the inhibitor of the DNA mismatch repair pathway inhibits one or more proteins of the DNA mismatch repair pathway. In some embodiments, the one or more proteins is selected from the group consisting of MLH1, PMS2 (or MutL alpha), PMS1 (or MutL beta). MLH3 (or MutL gamma), MutS alpha (MSH2-MSH6), MutS beta (MSH2-MSH3), MSH2, MSH6, PCNA, RFC, EXO1, POLδ, and PCNA. In certain embodiments, the one or more proteins is MLH1. In some embodiments, MLH1 comprises an amino acid sequence of SEQ ID NO: 204, or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 204.

The inhibitor utilized in the polynucleotide may be an antibody, a small molecule, a small interfering RNA (siRNA), a small non-coding microRNA, or a dominant negative variant of an MMR protein that inhibits the activity of a wild type MMR protein (e.g., a dominant negative variant of (MLH1). In certain embodiments, the inhibitor is an antibody that inhibits the activity of one or more proteins of the DNA mismatch repair pathway. In some embodiments, the inhibitor is a small molecule that inhibits the activity of one or more proteins of the DNA mismatch repair pathway. In certain embodiments, the inhibitor is a small interfering RNA (siRNA) or a small non-coding microRNA that inhibits the activity of one or more proteins of the DNA mismatch repair pathway. In some embodiments, the inhibitor is a dominant negative variant of MLH1 that inhibits MLH1.

In some embodiments, the dominant negative variant is (a) MLH1 E34A (SEQ ID NO: 222), (b) MLH1 Δ756 (SEQ ID NO: 208), (c) MLH1 Δ754-756 (SEQ ID NO: 209), (d) MLH1 E34A Δ754-756 (SEQ ID NO: 210), (e) MLH1 1-335 (SEQ ID NO: 211), (f) MLH1 1-335 E34A (SEQ ID NO: 212), (g) MLH1 1-335 NLS^{SV40} (SEQ ID NO: 213), (h) MLH1 501-756 (SEQ ID NO: 215), (i) MLH1 501-753 (SEQ ID NO: 216), (j) MLH1 461-753 (SEQ ID NO: 218), or (k) NLS^{SV40} MLH1 501-753 (SEQ ID NO: 223), or a polypeptide comprising an amino acid sequence having at least 70%, at least 75%, at least 80%, at least 85%, at least 90%, at least 95%, at least 96%, at least 97%, at least 98%, at least 99%, or up to and including 100% sequence identity with any of SEQ ID NOs: 208-213, 215, 216, 218, 222, or 223.

The prime editors utilized in the polynucleotides of the present disclosure comprise multiple components (e.g., a napDNAbp and a polymerase). In some embodiments, the napDNAbp is a nuclease active Cas9 domain, a nuclease inactive Cas9 domain, or a Cas9 nickase domain or variant thereof. In certain embodiments, the napDNAbp is selected from the group consisting of: Cas9, Cas12e, Cas12d, Cas12a, Cas12b1, Cas13a, Cas12c, Cas12b2. Cas13a, Cas12c, Cas12d, Cas12e, Cas12h, Cas12i, Cas12g, Cas12f (Cas14), Cas12f1, Cas12j (CasΦ), and Argonaute and optionally has a nickase activity. In certain embodiments, the napDNAbp comprises an amino acid sequence of any one of SEQ ID NOs: 2, 4-67, or 99 (PEmax) or an amino acid sequence having at least an 80%, 85%, 90%, 95%, or 99% sequence identity with any one of SEQ ID NOs: 2, 4-67, or 99 (PEmax). In certain embodiments, the napDNAbp comprises an amino acid sequence of SEQ ID NO: 2 or SEQ ID NO: 37 (i.e., the napDNAbp of PE1 and PE2) or an amino acid sequence having at least an 80%, 85%, 90%, 95%, or 99% sequence identity with SEQ ID NO: 2 or SEQ ID NO: 37. In some embodiments, the polymerase is a DNA-dependent DNA polymerase or an RNA-dependent DNA polymerase. In some embodiments, the polymerase is a reverse transcriptase. In certain embodiments, the reverse transcriptase comprises an amino acid sequence of any one of SEQ ID NOs: 69-98 or an amino acid sequence having at least an 80%, 85%, 90%, 95%, or 99% sequence identity with any one of SEQ ID NOs: 69-98.

The napDNAbp and the polymerase of the prime editor may be joined together to form a fusion protein. In some embodiments, the napDNAbp and the polymerase of the prime editor are joined by a linker to form a fusion protein. In certain embodiments, the linker comprises an amino acid sequence of any one of SEQ ID NOs: 102, 118-131, or an amino acid sequence having at least an 80%, 85%, 90%, 95%, or 99% sequence identity with any one of SEQ ID NOs: 102, 118-131. In some embodiments, the linker is 1, 2, 3, 4, 5, 6, 7, 8, 9, 10, 11, 12, 13, 14, 15, 16, 17, 18, 19.20, 21, 22.23, 24, 25, 26, 27.28, 29, 30.31, 32, 33, 34, 35.36, 38, 38, 39, 40, 41, 42, 43, 44, 45, 46, 47, 48, 49, or 50 amino acids in length.

The polynucleotides disclosed herein may comprise vectors. In some embodiments, the polynucleotide is a DNA vector. In certain embodiments, the DNA vector is an AAV or lentivirus DNA vector. In some embodiments, the AAV vector is serotype 1, 2, 3, 4, 5, 6, 7, 8, 9, or 10.

The prime editors encoded by the presently disclosed polynucleotides may also be further joined to additional components. In some embodiments, the prime editor as a fusion protein is further joined by a second linker to the inhibitor of the DNA mismatch repair pathway. In certain embodiments, the second linker comprises a self-hydrolyzing linker. In certain embodiments, the second linker comprises an amino acid sequence of any one of SEQ ID NOs: 102, 118-131, or 233-236, or an amino acid sequence having at least an 80%, 85%, 90%, 95%, or 99% sequence identity with any one of SEQ ID NOs: 102, 118-131, or 233-236. In some embodiments, the second linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In some embodiments, the one or more modifications to the nucleic acid molecule installed at the target site comprise one or more transitions, one or more transversions, one or more insertions, one or more deletions, or one more inversions. In certain embodiments, the one or more transitions are selected from the group consisting of: (a) T to C; (b) A to G; (c) C to T; and (d) G to A. In certain embodiments, the one or more transversions are selected from the group consisting of: (a) T to A; (b) T to G; (c) C to G; (d) C to A; (e) A to T; (f) A to C; (g) G to C; and (h) G to T. In certain embodiments, the one or more modifications comprises changing (1) a G:C basepair to a T:A basepair, (2) a G:C basepair to an A:T basepair, (3) a G:C basepair to a C:G basepair, (4) a T:A basepair to a G:C basepair, (5) a T:A basepair to an A:T basepair, (6) a T:A basepair to a C:G basepair, (7) a C:G basepair to a G:C basepair, (8) a C:G basepair to a T:A basepair, (9) a C:G basepair to an A:T basepair, (10) an A:T basepair to a T:A basepair, (11) an A:T basepair to a G:C basepair, or (12) an A:T basepair to a C:G basepair. In some embodiments, the one or more modifications comprises an insertion or deletion of 1, 2, 3, 4, 5, 6, 7, 8, 9, 10, 11, 12, 13, 14, 15, 16, 17, 18, 19, 20, 21, 22, 23, 24, or 25 nucleotides.

The polynucleotides of the present disclosure may be used for making corrections to one or more disease-associated genes. In some embodiments, the one or more modifications comprises a correction to a disease-associated gene. In certain embodiments, the disease-associated gene is associated with a polygenic disorder selected from the group consisting of: heart disease; high blood pressure; Alzheimer's disease; arthritis; diabetes; cancer, and obesity. In certain embodiments, the disease-associated gene is associated with a monogenic disorder selected from the group consisting of: Adenosine Deaminase (ADA) Deficiency; Alpha-1 Antitrypsin Deficiency; Cystic Fibrosis; Duchenne Muscular Dystrophy; Galactosemia; Hemochromatosis; Huntington's Disease; Maple Syrup Urine Disease; Marfan Syndrome; Neurofibromatosis Type 1; Pachyonychia Congenita; Phenylketonuria; Severe Combined Immunodeficiency; Sickle Cell Disease; Smith-Lemli-Opitz Syndrome; a trinucleotide repeat disorder; a prion disease; and Tay-Sachs Disease.

In another aspect, the present disclosure provides cells. In some embodiments, the cell comprises any of the polynucleotides described herein.

In another aspect, the present disclosure provides pharmaceutical compositions. In some embodiments, the pharmaceutical composition comprises any of the compositions disclosed herein. In some embodiments, the pharmaceutical composition comprises any of the compositions disclosed herein and a pharmaceutically acceptable excipient. In some embodiments, the pharmaceutical composition comprises any of the polynucleotides disclosed herein. In some embodiments, the pharmaceutical composition comprises any of the polynucleotides disclosed herein and a pharmaceutically acceptable excipient.

In another aspect, the present disclosure provides kits. In some embodiments, the kit comprises any of the compositions disclosed herein, a pharmaceutical excipient, and instructions for editing a DNA target site by prime editing. In some embodiments, the kit comprises any of the polynucleotides disclosed herein, a pharmaceutical excipient, and instructions for editing a DNA target site by prime editing.

The present disclosure also provides methods and pegRNAs for prime editing whereby correction by the MMR pathway of the alterations introduced into a target nucleic acid molecule is evaded, without the need to provide an inhibitor of the MMR pathway. Surprisingly, pegRNAs designed with consecutive nucleotide mismatches compared to the endogenous sequence of a target site on a target nucleic acid, for example, pegRNAs that have three or more consecutive mismatching nucleotides, can evade correction by the MMR pathway, resulting in an increase in prime editing efficiency and a decrease in the frequency of indel formation compared to the introduction of a single nucleotide mismatch using prime editing. In addition, insertions or deletions of consecutive nucleotides at the target site of the target nucleic acid, for example, insertions or deletions greater than 10 nucleotides in length, introduced by prime editing also evade correction by the MMR pathway, resulting in an increase in prime editing efficiency and a decrease in the frequency of indel formation compared to the introduction of an insertion or deletion of less than 10 nucleotides in length using prime editing.

Thus, in another aspect, the present disclosure provides methods for editing a nucleic acid molecule by prime editing comprising contacting a nucleic acid molecule with a prime editor (e.g., PE2, PE3, or any of the other prime editors described herein) and a pegRNA with a DNA synthesis template on its extension arm comprising three or more consecutive nucleotide mismatches relative to the endogenous sequence of a target site on the nucleic acid molecule. In some embodiments, at least one of the consecutive nucleotide mismatches results in an alteration in the amino acid sequence of a protein expressed from the nucleic acid molecule, while at least one of the remaining nucleotide mismatches is a silent mutation. The silent mutations may be in coding regions of the target nucleic acid molecule (i.e., in a part of a gene that encodes a protein), or the silent mutations may be in non-coding regions of the target nucleic acid molecule. In some embodiments, when the silent mutations are in a coding region, the silent mutations introduce into the nucleic acid molecule one or more alternate codons encoding the same amino acid as the unedited nucleic acid molecule. In some embodiments, when the silent mutations are in a non-coding region, the silent mutations are present in a region of the nucleic acid molecule that does not influence splicing, gene regulation, RNA lifetime, or other biological properties of the target site on the nucleic acid molecule.

Any number of consecutive nucleotide mismatches compared to the sequence of the target site can be designed in the DNA synthesis template of a pegRNA to achieve the benefits of evading correction by the MMR pathway, and thereby increase prime editing efficiency and/or reduce indel formation. In some embodiments, the DNA synthesis template comprises at least three consecutive nucleotide mismatches compared to the sequence of the target site. In some embodiments, the DNA synthesis template of the extension arm on the pegRNA comprises 2, 3, 4, 5, 6, 7, 8, 9, 10, 11, 12, 13, 14, 15, 16, 17, 18, 19, 20, 21, 22, 23, 24, or 25 consecutive nucleotide mismatches relative to the endogenous sequence of a target site in the nucleic acid molecule edited by prime editing. In some embodiments, the DNA synthesis template of the extension arm on the pegRNA comprises three or more, four or more, five or more, six or more, seven or more, eight or more, nine or more, or ten or more consecutive nucleotide mismatches relative to the endogenous sequence of a target site on the nucleic acid molecule. In certain embodiments, the use of three or more consecutive nucleotide mismatches results in an increase in prime editing efficiency by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold relative to a method using a pegRNA comprising a DNA synthesis template comprising only one consecutive nucleotide mismatch relative to the endogenous sequence of a target site on the nucleic acid molecule. In certain embodiments, the use of three or more consecutive nucleotide mismatches results in a decrease in the frequency of indel formation by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold relative to a method using a peg RNA comprising a DNA synthesis template comprising only one consecutive nucleotide mismatch relative to the endogenous sequence of a target site on the nucleic acid molecule.

In another aspect, the present disclosure provides methods for editing a nucleic acid molecule by prime editing comprising contacting a nucleic acid molecule with a prime editor (e.g., PE2, PE3, or any of the other prime editors described herein) and a pegRNA with a DNA synthesis template on its extension arm comprising an insertion or deletion of 10 or more contiguous nucleotides relative to the endogenous sequence of a target site on the nucleic acid molecule. In some embodiments, the DNA synthesis template of a pegRNA can be designed to introduce insertions or deletions greater than 3 nucleotides to avoid or reduce the impact of mismatch correction by the cellular MMR pathway, thereby improving prime editing efficiency. In some embodiments, the DNA synthesis template of the pegRNA is designed to introduce one or more insertions and/or deletions of 3, 4, 5, 6, 7, 8, 9, 10, or more contiguous nucleotides to avoid or reduce the impact of mismatch correction by the cellular MMR pathway, thereby improving prime editing efficiency. In some embodiments, insertions or deletions of any length greater than 10 contiguous nucleotides can be used to achieve the benefits of evading correction by the MMR pathway. In some embodiments, the DNA synthesis template comprises an insertion of 3, 4, 5, 6, 7, 8, 9, 10, 11, 12, 13, 14, 15, 16, 17, 18, 19, 20, 21, 22, 23, 24, or 25 contiguous nucleotides relative to the endogenous sequence of a target site on a nucleic acid molecule edited by prime editing. In some embodiments, the DNA synthesis template comprises a deletion of 3, 4, 5, 6, 7, 8, 9, 10, 11, 12, 13, 14, 15, 16, 17, 18, 19, 20, 21, 22, 23, 24, or 25 contiguous nucleotides relative to the endogenous sequence of a target site on a nucleic acid molecule edited by prime editing. In some embodiments, the DNA synthesis template comprises an insertion of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous nucleotides relative to the endogenous sequence of a target site on a nucleic acid molecule edited by prime editing. In some embodiments, the DNA synthesis template comprises a deletion of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous nucleotides relative to the endogenous sequence of a target site on a nucleic acid molecule edited by prime editing. In some embodiments, the DNA synthesis template comprises an insertion or deletion of 11 or more contiguous nucleotides, 12 or more contiguous nucleotides, 13 or more contiguous nucleotides, 14 or more contiguous nucleotides, 15 or more contiguous nucleotides, 16 or more contiguous nucleotides, 17 or more contiguous nucleotides, 18 or more contiguous nucleotides, 19 or more contiguous nucleotides, 20 or more contiguous nucleotides, 21 or more contiguous nucleotides, 22 or more contiguous nucleotides, 23 or more contiguous nucleotides, 24 or more contiguous nucleotides, or 25 or more contiguous nucleotides relative to a target site on a nucleic acid molecule. In certain embodiments, the DNA synthesis template comprises an insertion or deletion of 15 or more contiguous nucleotides relative to the endogenous sequence of a target site on the nucleic acid molecule.

In some embodiments, prime editing with a pegRNA designed to introduce an insertion and/or deletion of multiple contiguous nucleotides, for example, three or more contiguous nucleotides, relative to the endogenous sequence of a target site results in an increase in prime editing efficiency compared to prime editing with a corresponding control pegRNA (e.g., a control pegRNA that does not introduce an insertion or deletion of three or more contiguous nucleotides) by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold. In some embodiments, prime editing with a pegRNA designed to introduce an insertion or deletion of 3, 4, 5, 6, 7, 8, 9, 10, or more contiguous nucleotides relative to the endogenous sequence of a target site results in an increase in prime editing efficiency relative to prime editing with a corresponding control pegRNA (e.g., a control pegRNA that does not introduce insertion or deletion of the three or more contiguous nucleotides) by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold. In some embodiments, making an insertion or deletion of 10 or more contiguous nucleotides results in an increase in prime editing efficiency by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold relative to a method using a pegRNA comprising a DNA synthesis template comprising an insertion or deletion of fewer than 10 nucleotides relative to the endogenous sequence of a target site on the nucleic acid molecule. In some embodiments, making an insertion or deletion of 10 or more nucleotides results in a decrease in the frequency of indel formation by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold relative to a method using a pegRNA comprising a DNA synthesis template comprising an insertion or deletion of fewer than 10 nucleotides relative to the endogenous sequence of a target site on the nucleic acid molecule.

In another aspect, the present disclosure also provides pegRNAs useful for editing a nucleic acid molecule by prime editing while evading correction by the MMR pathway of the alterations introduced into the nucleic acid molecule, thereby increasing prime editing efficiency and/or reducing indel formation. In some embodiments, the extension arm of the pegRNAs provided by the present disclosure comprise three or more consecutive nucleotide mismatches relative to the endogenous sequence of a target site on the nucleic acid molecule. In some embodiments, at least one of the three consecutive nucleotide mismatches relative to the endogenous sequence of the target site is a silent mutation. In some embodiments, at least one of the consecutive nucleotide mismatches results in an alteration in the amino acid sequence of a protein expressed from the target nucleic acid molecule, while at least one of the remaining nucleotide mismatches is a silent mutation. The silent mutations may be in coding regions of the target nucleic acid molecule (i.e., in a part of a gene that encodes a protein), or the silent mutations may be in non-coding regions of the target nucleic acid molecule. In some embodiments, when the silent mutations are in a coding region, the silent mutations introduce into the nucleic acid molecule one or more alternate codons encoding the same amino acid as the unedited nucleic acid molecule. In some embodiments, when the silent mutations are in a non-coding region, the silent mutations are present in a region of the nucleic acid molecule that does not influence splicing, gene regulation, RNA lifetime, or other biological properties of the target site on the nucleic acid molecule.

Any number of consecutive nucleotide mismatches of three or more can be incorporated into the extension arm of the pegRNAs described herein to achieve the benefits of evading correction by the MMR pathway. In some embodiments, the DNA synthesis template of the extension arm of the pegRNA comprises 2, 3, 4, 5, 6, 7, 8, 9, 10, 11, 12, 13, 14, 15, 16, 17, 18, 19, 20, 21, 22, 23, 24, or 25 consecutive nucleotide mismatches relative to the endogenous sequence of a target site on the nucleic acid molecule edited by prime editing. In some embodiments, the DNA synthesis template of the extension arm of the pegRNA comprises at least three consecutive nucleotide mismatches relative to the endogenous sequence of a target site on the nucleic acid molecule edited by prime editing. In some embodiments, the DNA synthesis template of the extension arm of the pegRNA comprises 3, 4, 5, 6, 7, 8, 9, 10, 11, 12, 13, 14, 15, 16, 17, 18, 19, or 20 consecutive nucleotide mismatches relative to the endogenous sequence of a target site on the nucleic acid molecule edited by prime editing. In some embodiments, the DNA synthesis template of the extension arm of the pegRNA comprises 3, 4, 5, 6, 7, 8, 9, or 10 consecutive nucleotide mismatches relative to the endogenous sequence of a target site on the nucleic acid molecule edited by prime editing. In some embodiments, the DNA synthesis template of the extension arm on the pegRNA comprises three or more, four or more, five or more, six or more, seven or more, eight or more, nine or more, or ten or more consecutive nucleotide mismatches relative to the endogenous sequence of a target site on the nucleic acid molecule. In certain embodiments, the presence of three or more consecutive nucleotide mismatches on the extension arm of the pegRNA results in an increase in prime editing efficiency by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold relative to a pegRNA comprising a DNA synthesis template comprising only one consecutive nucleotide mismatch relative to the endogenous sequence of a target site on the nucleic acid molecule. In certain embodiments, the use of three or more consecutive nucleotide mismatches results in a decrease in the frequency of indel formation by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold relative to a pegRNA comprising a DNA synthesis template comprising only one consecutive nucleotide mismatch relative to the endogenous sequence of a target site on the nucleic acid molecule.

In another aspect, the present disclosure provides a prime editor system for site specific genome modification comprising (a) a prime editor comprising (i) a nucleic acid programmable DNA binding protein (napDNAbp) and (ii) a DNA polymerase, and (b) an inhibitor of the DNA mismatch repair pathway. In some embodiments, the inhibitor of the DNA mismatch repair pathway inhibits one or more proteins of the DNA mismatch repair pathway (e.g., MLH1, PMS2 (or MutL alpha), PMS1 (or MutL beta), MLH3 (or MutL gamma). MutS alpha (MSH2-MSH6), MutS beta (MSH2-MSH3), MSH2, MSH6, PCNA, RFC, EXO1, POLδ, and/or PCNA). In some embodiments, the one or more proteins is MLH1. In certain embodiments, the MLH1 comprises an amino acid sequence of SEQ ID NO: 204, or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 204.

Any inhibitor of the DNA mismatch repair pathway may be used in the systems described herein. In some embodiments, the inhibitor is an antibody that inhibits the activity of one or more proteins of the DNA mismatch repair pathway. In some embodiments, the inhibitor is a small molecule that inhibits the activity of one or more proteins of the DNA mismatch repair pathway. In some embodiments, the inhibitor is a small interfering RNA (siRNA) or a small non-coding microRNA that inhibits the activity of one or more proteins of the DNA mismatch repair pathway. In some embodiments, the inhibitor is a dominant negative variant of an MMR protein that inhibits the activity of a wild type MMR protein (e.g., a dominant negative variant of MLH1 that inhibits MLH1).

In certain embodiments, the dominant negative variant used in the systems of the present disclosure is (a) MLH1 E34A (SEQ ID NO: 222), (b) MLH1 Δ756 (SEQ ID NO: 208), (c) MLH1 Δ754-756 (SEQ ID NO: 209), (d) MLH1 E34A Δ754-756 (SEQ ID NO: 210), (e) MLH1 1-335 (SEQ ID NO: 211), (f) MLH1 1-335 E34A (SEQ ID NO: 212), (g) MLH1 1-335 NLS^{SV40} (SEQ ID NO: 213), (h) MLH1501-756 (SEQ ID NO: 215), (i) MLH1501-753 (SEQ ID NO: 216), (j) MLH1 461-753 (SEQ ID NO: 218), or (k) NLS^{SV40} MLH1 501-753 (SEQ ID NO: 223), or a polypeptide comprising an amino acid sequence having at least 70%, at least 75%, at least 80%, at least 85%, at least 90%, at least 95%, at least 96%, at least 97%, at least 98%, at least 99%, or up to and including 100% sequence identity with any of SEQ ID NOs: 208-213, 215, 216, 218, 222, or 223. The present disclosure also contemplates methods for performing prime editing on a nucleic acid molecule in a cell in which MMR activity is knocked out entirely (e.g., by knocking down one or more genes involved in the MMR pathway in the genome of the cell). Such methods provide the benefits of inhibiting MMR (e.g., improved editing efficiency and decreased indel formation) without the need to provide an inhibitor of MMR. Thus, in another aspect, the present disclosure provides methods for editing a nucleic acid molecule by prime editing comprising: contacting a nucleic acid molecule with a prime editor and a pegRNA, thereby installing one or more modifications to the nucleic acid molecule at a target site, wherein the nucleic acid molecule is in a cell comprising a knockout of one or more genes involved in the DNA mismatch repair (MMR) pathway. In some embodiments, the method further comprises contacting the nucleic acid molecule with a second strand nicking gRNA. In certain embodiments, the prime editing efficiency is increased by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold relative to a method performed in a cell that does not comprise a knockout of one or more genes involved in MMR. In certain embodiments, the frequency of indel formation is decreased by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, or at least 10.0-fold relative to a method performed in a cell that does not comprise a knockout of one or more genes involved in MMR. In some embodiments, the one or more genes involved in MMR is selected from the group consisting of genes encoding the proteins MLH1, PMS2 (or MutL alpha), PMS1 (or MutL beta), MLH3 (or MutL gamma), MutS alpha (MSH2-MSH6), MutS beta (MSH2-MSH3), MSH2, MSH6, PCNA, RFC, EXO1, POLδ, and PCNA. In certain embodiments, the one or more genes is the gene encoding MLH1 (e.g., comprising an amino acid sequence of SEQ ID NO: 204, or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 204).

In another aspect, the present disclosure provides methods for editing a nucleic acid molecule by prime editing comprising: contacting a nucleic acid molecule with a prime editor, a pegRNA, and an inhibitor of p53, thereby installing one or more modifications to the nucleic acid molecule at a target site. In some embodiments, the method further comprises contacting the nucleic acid molecule with a second strand nicking gRNA.

In some embodiments, the prime editing efficiency is increased by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, or at least 75-fold, in the presence of the inhibitor of p53. In some embodiments, the frequency of indel formation is decreased by at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, or at least 75-fold, in the presence of the inhibitor of p53.

In some embodiments, the inhibitor of p53 is a protein. In certain embodiments, the inhibitor of p53 is the protein i53. In some embodiments, the inhibitor of p53 is an antibody that inhibits the activity of p53. In some embodiments, the inhibitor of p53 is a small molecule that inhibits the activity of p53. In some embodiments, the inhibitor of p53 is a small interfering RNA (siRNA) or a small non-coding microRNA that inhibits the activity of p53.

In another aspect, the present disclosure describes improved prime editor fusion proteins, including PEmax of SEQ ID NO: 99. The disclosure also contemplates fusion proteins having an amino acid sequence with a sequence identity of at least 80%, at least 85%, at least 90%, at least 95%, at least 96%, at least 97%, at least 98%, at least 99%, or at least up to 100% with SEQ ID NO: 99.

The inventors have surprisingly found that the editing efficiency of prime editing may be significantly increased (e.g., 2-fold increase, 3-fold increase, 4-fold increase, 5-fold increase, 6-fold increase, 7-fold increase, 8-fold increase, 9-fold increase, or 10-fold increase or more) when one or more components of the canonical prime editor fusion protein (i.e., PE2) are modified. Modifications may include a modified amino acid sequence of one or more components (e.g., a Cas9 component, a reverse transcriptase component, or a linker).

In other aspects, the present disclosure also provides compositions and pharmaceutical compositions comprising PEmax, methods of prime editing using PEmax, polynucleotides and vectors encoding PEmax, and kits and cells comprising PEmax.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 provides a schematic showing that prime editing enables guide RNA-templated genomic manipulations. DNA prime editing intermediates capable of being repaired by cellular factors are shown in boxes.

FIG. 2 provides a schematic for a DNA repair CRISPRi screen for prime editing outcomes.

FIGS. 3A-3C show optimization of prime editing efficiency at the target site. FIG. 3A provides a schematic for the optimization process. FIG. 3B shows percent reads with a specified modification at a target site in HeLa cells. FIG. 3C shows percent reads with a specified modification at a target site with blasticidin selection in HeLa cells.

FIGS. 4A-4B show a prime editing CRISPRi screen with a DNA repair library. FIG. 4A provides a schematic of the screening process. FIG. 4B shows percent reads with a specified modification in bulk editing of post-screen HeLa cells.

FIGS. 5A-5B show that the CRISPRi screen reveals that DNA mismatch repair limits prime editing efficiency. Knockdown of mismatch repair proteins (MSH2. MSH6, PMS2, and MLH1) improves the efficiency of PE2 by 3-fold and PE3 by 2-fold.

FIGS. 6A-6C show that siRNA knockdown of MMR improves prime editing in HEK293T cells. Editing results at multiple endogenous loci validate the findings of the CRISPRi screen.

FIGS. 7A-7B show that complete MMR knockout dramatically enhances prime editing. In the absence of MMR, PE2 editing efficiency is shown to match PE3 editing efficiency.

FIG. 8A provides a schematic for the mechanism of mismatch repair (MMR). In the first step, MSH2:MSH6 (MutSα) binds the mismatch and recruits MLH1:PMS2 (MutLα). The DNA nick signals to MMR which strand to repair. In the second step, MutLα indiscriminately incises the nicked strand 5′ and 3′ of the mismatch. In the third step, EXO1 excises the mismatch from MutLα-generated nicks. In the fourth step, POLδ resynthesizes the excised strand, followed by LIG1 ligation.

FIG. 8B provides yet another schematic for the mechanism of mismatch repair, MMR, in eukaryotic cells. The left side of the schematic depicts 5′ MMR. (A) The MutS homolog proteins (MSH, purple) MutSα (MSH2-MSH6), or MutSβ (MSH2-MSH3) recognize and bind a mismatch. RPA bound to single-strand DNA prevents EXO1 from accessing and degrading DNA. (B) In the sliding clamp model, MutSα/β at a mismatch binds ATP and undergoes nucleotide switch activation, becoming a sliding clamp that diffuses along the DNA. Multiple MSH clamps are loaded at a single mismatch. The interaction of EXO1 with MSH sliding clamps overcomes the RPA barrier and activates EXO1 for 5′ to 3′ excision from the 5′ nick. MutL homolog proteins (MLK) (MutLα is ScMlh1-Pms1 or HsMLH1-PMS2) bind ATP and may interact with MSH sliding clamps, though MLH is not absolutely required in vitro for 5′ MMR. In other models, MSH remains at the mismatch to authorize excision or can load multiple MLH clamps onto the DNA in the vicinity of the mismatch (not shown). (C) In the sliding clamp model, the EXO1/MSH complex dissociates after excising several hundred nucleotides. Iterative rounds of MSH-EXO1 excision create an excision tract coated with RPA that extends from the 5′ nick to just beyond the mismatch. MLH may limit excision by modulating the number of MSH clamps on DNA. (D) RFC (not shown) loads PCNA clamps with specific orientation at 3′ termini of strand breaks or gaps, and PCNA facilitates high-fidelity DNA synthesis by Pol δ or ε. (E) DNA ligase I seals the nick. The right side of the schematic depicts 3′ MMR. (A) MSH recognizes a mismatch. (B) In the sliding clamp model, ATP-dependent binding and nucleotide switching creates MSH sliding clamps that diffuse from the mismatch. The interaction of ATP-bound MLH heterodimers with MSH sliding clamps and PCNA oriented with respect to 3′ termini activates MLH strand-specific nicking. Alternatively, ATP-activated MSH may remain at the mismatch to load MLH and activate nicking (not shown). (C) Excision is EXO1-dependent or -independent, leading to an RPA-coated excision track. An EXO1-independent Pol δ strand-displacement pathway is not shown. (D) Pol δ or ε with the aid of PCNA completes gap filling. (E) DNA ligase I seals the nick.

FIGS. 9A-9C provide a schematic of mismatch repair of PE2 intermediates. MMR inhibition provides additional time for flap ligation, removing the strand discrimination signal for repair of the heteroduplex.

FIG. 10 shows that expression of dominant-negative MLH1 mutants boosts PE2 efficiency. MLH1 dominant-negative mutants improve PE2 efficiency by 2- to 4-fold. RNF2+3 G to C is not responsive to MMR-inhibition.

FIGS. 11A-11B show the effect of MLH1 mutants on PE3. MLH1 mutants reduce PE3 indels by half.

FIGS. 12A-12B show that MLH1 mutant improvements translate to other sites. FIG. 12A shows that PE2 editing efficiency increases with MLH1 mutants, and only RNF2+3 G to C is resistant to MMR-inhibition. FIG. 12B shows that MLH1 mutants reduce the occurrence of indels by half.

FIG. 13 provides a schematic showing mismatch repair of PE3 intermediates.

FIG. 14 provides a schematic showing that mismatch repair differentially resolves PE3 intermediates. Mismatch repair is required for the one edit-favored intermediate.

FIG. 15A-15H show screening of MLH1 mutants for smaller size and improved activity. FIG. 15A shows that MLH1 Δ754-756 most strongly promotes PE2 editing (hereafter named MLH1dn). MLH1 N-terminal domain approaches the effectiveness of MLH1dn (hereafter named MLH1^{NTD}). MLH1 dominant negative mutants may function by saturating binding of MutS. FIG. 15B shows that the MLH1 N-terminal domain+NLS approaches the activity of MLH1neg. FIG. 15C shows that MLH1dn fusion to PE by a self-cleavable P2A linker (PE-2A-MLH1dn) can improve prime editing efficiency. FIGS. 15D-15F show that MMR KD phenocopies MLH1neg expression. FIGS. 15G-15H show that the efficiency of PE2 and PE3 is equal in the absence of MMR, suggesting that the complementary nick only serves to bias MMR.

FIG. 16 shows that MLH1dn reduces indels for PE3. Silent pegRNA is pegRNA that does not encode an edit or produce a mismatch. MLH1dn only reduces PE3 indels if a mismatch is generated.

FIG. 17 show that mismatch repair of PE heteroduplexes produces a diffuse indel pattern. Indel distribution is broad for PE3 for these edits, but inhibiting MMR with MLH1dn narrows that distribution. This suggests that MMR makes incisions after mismatch recognition that contribute to the indels generated by PE3.

FIG. 18 shows mismatch repair of PE3 intermediates.

FIGS. 19A-19B show that MMR excision of the target locus generates indels in PE3.

FIGS. 20A-20B show that MMR knockdown or knockout has no effect on RNF2+3 G to C. This suggests that the RNF2 site is not repaired by MMR or the resulting C:C mismatch is not repaired by MMR.

FIGS. 21A-21C show that other substitution edits at RNF2 can be improved with MLH1dn.

FIGS. 22A-22B show that MLH1dn improves substitution edits at other sites, including HEK3. MLH1dn strongly enhances PE2 editing and lowers PE3 indels.

FIGS. 23A-23D show that MLH1dn improves substitution edits at other sites, including FANCF. MLH1dn strongly enhances PE2 editing and lowers PE3 indels.

FIGS. 24A-24B show that PE improvement by MHL1dn is mismatch dependent. MLH1dn increases PE2 editing by 2-fold on average in HEK293T cells. FIG. 24A shows that G to C edits (C:C mismatches) are unaffected by MMR in HEK293T cells. This suggests that G to C edits have a higher baseline efficiency than other substitutions. FIG. 24B shows a substantial increase in the ratio of edit:indel purity from MLH1dn used with PE3, which is also mismatch dependent.

FIGS. 25A-25D show that MLH1dn also improves the efficiency of small insertion and deletion edits. MMR is known to repair insertions and deletions <15 nucleotides in length.

FIGS. 26A-26B show that MLH1dn reduced pegRNA scaffold integration. Scaffold integration events at these sites occur through a double-strand break (DSB) intermediate.

FIG. 27 shows that MLH1dn does not promote substantial PE off-target editing. Small increases in off-target (OT) editing were observed at the HEK4 off-target site 3.

FIGS. 28A-28B show that MLH1dn does not induce detectable microsatellite instability at biomarker loci. MMR inhibition is known to cause shortening of homopolymer microsatellite regions.

FIG. 29 shows that MLH1dn offers a method to increase prime editing efficiency at sites without good ngRNAs, such as HEK4.

FIG. 30 shows that MLH1dn improves PE at disease sites.

FIG. 31 shows that MLH1dn enhances installation of the protective APOE Christchurch allele in mouse astrocytes. A 50% boost in editing efficiency and a large reduction in indels is shown.

FIG. 32 shows that HEK293T cells are MMR-compromised. The MLH1 promoter is hypermethylated in HEF293T, resulting in lower MLH1 expression.

FIGS. 33A-33B show that MLH1dn enhances prime editing in HeLa cells. FIG. 33A shows prime editing with PE2. FIG. 33B shows prime editing with PE3.

FIGS. 34A-34B show that MLH1dn enhances prime editing in HeLa cells. FIG. 34A shows editing of PRNP +6 G to T. FIG. 34B shows editing of APOE +6 G to T and +10 C to A.

FIGS. 35A-35B show that MLH1dn has a larger effect in MMR competent cell lines like HeLa.

FIGS. 36A-36D show that MLH1dn improvements synergize with stabilized pegRNAs.

FIGS. 37A-37B show that contiguous substitutions are useful as another strategy for evading MMR.

FIG. 38 shows that MMR does not efficiently repair 3 or more contiguous substitutions. Contiguous substitutions therefore offer a method for circumventing MMR and boosting PE efficiency.

FIGS. 39A-39C show that MLH1neg improves PE in HeLa cells.

FIGS. 40A-40G show that pooled Repair-seq CRISPRi screens reveal genetic determinants of substitution on prime editing outcomes. FIG. 40A shows that prime editing with the PE2 system is mediated by the PE2 enzyme (Streptococcus pyogenes Cas9 (SpCas9) H840A nickase fused to a reverse transcriptase) and a prime editing guide RNA (pegRNA). The PE3 system uses an additional single guide RNA (sgRNA) to nick the non-edited strand and yield higher editing efficiency. PBS, primer binding site. RT template, reverse transcription template. FIG. 40B provides an overview of prime editing Repair-seq CRISPRi screens. A library of CRISPRi sgRNAs and a pre-validated prime edit site are transduced into CRISPRi cell lines and transfected with prime editors targeting the edit site. CRISPRi sgRNA identities and prime edited sites are amplified together from genomic DNA and paired-end sequenced together to link each genetic perturbation with editing outcome. SaCas9, Staphylococcus aureus Cas9. FIG. 40C shows the effect of each CRISPRi sgRNA on the percentage of sequencing reads reporting the intended G⋅C-to-C⋅G prime edit at the targeted edit site in pooled CRISPRi screens. Each value depicts all sequencing reads carrying the same CRISPRi sgRNA. FIG. 40D shows the effect of CRISPRi sgRNAs on editing efficiency in all screen conditions. Black dots represent individual non-targeting sgRNAs, black lines show the mean of all non-targeting sgRNAs, and gray shading represents kernel density estimates of the distributions of all sgRNAs. FIGS. 40E-40G show comparisons of gene-level effects of CRISPRi targeting on the intended G⋅C-to-C⋅G prime edit across different screen conditions. (FIG. 40E) K562 PE2 vs. HeLa PE2. (FIG. 40F) K562 PE3+50 vs. HeLa PE3+50. (FIG. 40G) K562 PE2 vs. K562 PE3+50. The effect of each gene is calculated as the average log 2 fold change in frequency from non-targeting sgRNAs for the two most extreme sgRNAs targeting the gene. Plotted quantities are the mean of n=2 independent biological replicates for each cell type, with bars showing the range of values spanned by the replicates. Black dots represent 20 random sets of three non-targeting sgRNAs.

FIGS. 41A-41J show genetic modulators of unintended prime editing outcomes. FIGS. 41A-41D show representative examples of four categories of unintended prime editing outcomes observed in CRISPRi screens. In each panel, the black bar depicts the sequence of an editing outcome, the blue bar depicts genomic sequence around the targeted editing site, and the orange bar depicts the pegRNA sequence. Blue and orange lines between the editing outcome and the genome or pegRNA depict local alignments between the outcome sequence and the relevant reference sequence. Mismatches in alignments are marked by X's, and insertions are marked by downward dimples. The location of the programmed edit is marked by a grey box. Red and cyan rectangles on the genome mark SaCas9 protospacers and PAMs, and black vertical lines mark the locations of SaCas9 nick sites. Orange, beige, grey, and red rectangles on the pegRNA mark the primer binding site (PBS), reverse transcription template (RTT), scaffold, and spacer, respectively. FIGS. 41E-41F provide a summary of editing outcome categories observed in PE2 screens (FIG. 41E) and in PE3+50 screens (FIG. 41F) in K562 cells. Plotted quantities are the mean±SD of all sgRNAs for each indicated gene (60 non-targeting sgRNAs, three sgRNAs per targeted gene), averaged across n=2 independent biological replicates. FIGS. 41G-41H show a comparison of the effects of knockdown of all genes targeted in CRISPRi screens on the frequency of joining of reverse transcribed sequence at unintended locations (FIG. 41G) or the frequency of deletions (FIG. 41H) from PE3+50. The effect of each gene is calculated as the average log 2 fold change in frequency from non-targeting sgRNAs for the two most extreme sgRNAs targeting the gene. Plotted quantities are the mean of n=2 independent biological replicates for each cell type, with bars showing the range of values spanned by the replicates. Black dots represent 20 random sets of three non-targeting sgRNAs. FIG. 41I shows the frequency of deletion as a function of genomic position relative to programmed PE3+50 nicks (dashed vertical lines) in K562 screen replicate I across all reads for indicated sets of CRISPRi sgRNAs (black line: 60 non-targeting sgRNAs; orange and green lines: three sgRNAs targeting each of MSH2, MSH6, MLH1, and PMS2) (top). Log 2 fold change in frequency of deletion as a function of genomic position from MSH2. MSH6, MLH1, and PMS2 sgRNAs compared to non-targeting sgRNAs (bottom). FIG. 41 J shows the effect of gene knockdowns on the fraction of all observed deletions that remove sequence at least 25-nt outside of programmed PE3+50 nicks in K562 screens. Each dot represents all reads for all sgRNAs targeting each gene. Black dots represent 20 sets of three random non-targeting sgRNAs.

FIGS. 42A-42D show a model for mismatch repair of prime editing intermediates. FIG. 42A shows a model for DNA mismatch repair (MMR) of PE2 intermediates. MMR excises and replaces the nicked strand during repair of the prime editor-generated heteroduplex substrate. Infrequent ligation of the nick before MMR recognition deprives the strand discrimination signal for MMR, resulting in un-biased resolution of the heteroduplex. FIG. 42B shows a model for MMR of PE3 intermediates. PE3 installs an additional nick on the non-edited strand that can direct MMR to replace the non-edited strand. Ligation of the edited strand nick leaves only the complementary-strand nick to signal repair by MMR, resulting in the desired prime editing outcome. FIG. 42C shows prime editing efficiencies of PE2 and PE3 prime editors at endogenous sites (HEK3, EMX1, and RUNX1) in HEK293T cells pre-treated with knockdown siRNAs against MSH2, MSH6, MLH1, or PMS2 transcripts. Cells were pre-transfected with siRNAs 3 days prior to transfection with prime editor components and siRNAs. Genomic DNA was harvested 3 days following transfection with prime editors and additional siRNA, then sequenced. Bars represent the mean of n=3 independent biological replicates. FIG. 42D shows prime editing efficiencies in HAP1 ΔMSH2 and HAP1 ΔMLH1 cells (mean of n=3 independent biological replicates). A, gene knockout.

FIGS. 43A-43F show that engineered dominant negative MMR proteins (dominant negative variants of MSH2, MSH6, PMS2, and MLH1) enhance prime editing. FIG. 43A shows editing improvement at HEK2, EMX1, and RUNX1 sites by co-expression of PE2 in trans with human MMR proteins or dominant negative variants in HEK293T cells. MMR proteins include MSH2, MSH6, PMS2, and MLH1. Dominant negative variants are designated as MSH2 K675R, MSH6 K1140R, PMS2 E41A, PMS2 E705K, MLH1 E34A, and MLH1 Δ756. All values from n=3 independent biological replicates are shown. FIG. 43B shows functional annotation of the 756-aa human MLH1 protein, including an ATPase domain, MSH2 interaction domain, NLS domain, PMS2 dimerization domain, and an endonuclease domain. FIG. 43C shows editing enhancement of MLH1 variants co-expressed with PE2 in HEK293T cells at HEK3, EMX1, and RUNX1 sites. Red boxes indicate mutations that inactivate MLH1 ATPase or endonuclease function. MLH1dn, MLH1 Δ754-756. MLH1NTD-NLS, codon-optimized MLH1 1-335-NLSSV40. All values from n=3 independent biological replicates are shown. FIG. 43D shows a comparison of the top three dominant negative MLH1 variants at additional prime edits. All values from n=3 independent biological replicates are shown. FIG. 43E shows prime editing with PE2 and MLH1 dn in trans, PE2 and MLH1^{NTD}-NLS in trans, and PE2-P2A-MLH1dn (human codon optimized) in HEK293T cells. Bars represent the mean of n=3 independent biological replicates. FIG. 43F compares the structure of PE2, PE3, PE4, and PE5. In particular, the PE4 editing system consists of a prime editor enzyme (nickase Cas9-RT fusion), MLH1dn, and pegRNA. The PE5 editing system consists of a prime editor enzyme, MLH1dn, pegRNA, and second-strand nicking sgRNA. FIG. 43G shows editing efficiencies of PE2, PE3, PE4, and PE5 systems in HEK293T cells. Bars represent the mean of n=3 independent biological replicates).

FIGS. 44A-44G show the characterization of PE4 and PE5 across diverse prime editing classes and cell types. FIG. 44A provides a summary of prime editing enhancement by PE4 and PE5 compared to PE2 and PE3 for 84 single-base substitution edits (seven for each substitution type) across seven endogenous sites in HEK293T cells. The grand mean±SD of all individual values of n=3 independent biological replicates are shown. FIG. 44B shows installation of single base mutations at the FANCF locus with PE2, PE3, PE4, and PE5 in HEK293T cells. Bars represent the mean of n=3 independent biological replicates. FIG. 44C shows that PE4 improves the 1- and 3-bp insertion and deletion prime edits compared to PE2 in HEK293T cells. Bars represent the mean of n=3 independent biological replicates. FIG. 44D shows PE4 editing enhancement over PE2 across 33 different insertion and deletion prime edits. Bars represent the mean of all individual values of n=3 independent biological replicates. FIGS. 44F-44F provide a summary of PE2 and PE4 editing efficiencies for 35 different substitutions of 1 to 5 contiguous bases at five endogenous sites in HEK293T cells. Seven pegRNAs were tested for each number of contiguous bases altered. The mean±SD of all individual values of n=3 independent biological replicates are shown. FIGS. 44G-44H show that installation of additional silent or benign mutations near the intended edit can increase editing efficiency by generating a heteroduplex substrate that evades MMR. The PAM sequence (NGG) for each target is underlined. The amino acid sequence of the targeted gene is centered above each DNA codon. Values represent the mean±SD of n=3 independent biological replicates. FIG. 44I shows a comparison of prime editing enhancement in different cell types. PE4 and PE5 systems enhance prime editing to a greater extent in MMR deficient cells (MMR−) than in MMR proficient cells (MMR+). The same set of 30 pegRNAs encoding single-base substitution edits were tested in HEK293T and HeLa cells. K562 and U2OS cells were edited with 10 pegRNAs that are a direct subset of the 30 pegRNAs tested in HEK293T and HeLa cells. The mean±SD of all individual values of sets of n=3 independent biological replicates are shown. P values were calculated using the Mann-Whitney U test. FIG. 44J shows prime editing with PE2, PE3, PE4, and PE5 in HeLa, K562, and U2OS cells. Bars represent the mean of n=3 independent biological replicates).

FIGS. 45A-45H show the effect of dominant negative MLH1 on prime editing product purity and off-targeting. FIG. 45A shows that edit-encoding pegRNAs program a base change within the nascent 3′ DNA flap and generate a heteroduplex following flap interconversion. Non-editing pegRNAs template a 3′ DNA flap with perfect complementarity to the genomic target site. FIG. 45B shows the frequency of indels from PE3 or PE5 with four edit-encoding pegRNAs that program single base mutations or four non-editing pegRNAs. Short horizontal bars indicate the mean of all individual values of sets of n=3 independent biological replicates. FIG. 45C shows the ratio of indel frequency from PE5 over PE3 with 4 edit-encoding pegRNAs that program single base mutations or four edit-encoding pegRNAs that program single base mutations or four non-editing pegRNAs. Short horizontal bars indicate the mean of all individual values of sets of n=3 independent biological replicates. FIG. 45D shows distribution of deletions at genomic target DNA formed by PE3 and PE5 using 12 substitution-encoding pegRNAs at endogenous DNMT1 and RNF2 loci in HEK293T cells. Dotted lines indicate position of pegRNA- and sgRNA-directed nicks. Data represent the mean±SD of n=3 independent biological replicates. FIG. 45E shows PE5/PE3 ratio of frequency of deletions that remove sequence greater than 25-nt outside of pegRNA- and sgRNA-directed nicks in HEK293T cells. Each dot represents one of 84 total pegRNAs that program substitution edits at a combined seven loci (mean of n=3 independent biological replicates). FIG. 45F shows PE5/PE3 ratio of frequency of editing outcomes with unintended pegRNA scaffold sequence incorporation or unintended flap rejoining in HEK293T cells. Each dot represents one of 84 total pegRNAs that program substitution edits at a combined seven loci (mean of n=3 independent biological replicates). FIG. 45G shows off-target prime editing by PE2 and PE4 in HEK293T cells. Bars represent the mean of n=3 independent biological replicates). FIG. 45H shows high-throughput sequencing analysis of 17 sensitive microsatellite repeat loci used for clinical diagnosis of MMR deficiency. HAP1 and HeLa cells are MMR-proficient, and HCT116 cells have impaired MMR. HAP1 ΔMSH2 cells underwent 60 cell divisions following MSH2 knockout. HeLa cells were transiently transfected with PE2 or PE4 components and incubated for 3 days before sequencing, wt, wild-type. All values from n=2 independent biological replicates are shown.

FIGS. 46A-46F show that PEmax architecture with PE4 and PE5 editing systems enhances editing at disease-relevant gene targets and cell types. FIG. 46A shows a schematic of PE2 and PEmax editor architectures. bpNLSSV40, bipartite SV40 NLS nuclear localization signal. MMLV RT, Moloney Murine Leukemia Virus reverse transcriptase pentamutant; codon opt., human codon-optimized. FIG. 46B shows that engineered pegRNAs (epegRNAs) contain a 3′ RNA structural motif that improve prime editing performance. FIG. 46C shows prime editing efficiencies of PE4 and PE5 combined with PEmax architectures and epegRNAs. Seven single-base substitution edits targeting different loci were tested in HeLa and HEK293T cells. Fold changes indicate the average of fold increases from each edit tested. The mean±SD of all individual values of n=3 independent biological replicates are shown. FIG. 46D shows prime editing at therapeutically-relevant sites in wild-type HeLa and HEK293T cells. The HBB locus is edited at the E6 codon commonly mutated in patients with sickle cell disease (E6V). The CDKL5 edit is at a site for which the c.1412delA mutation causes CDKL5 deficiency disorder, epegRNAs were used for editing the HBB, PRNP, and CDKL5 loci. Bars represent the mean of n=3 independent biological replicates. FIG. 46E shows correction of CDKL5 c.1412delA via an A⋅T insertion and a silent G⋅C-to-A⋅T edit in iPSCs derived from a patient heterozygous for the allele. Editing efficiencies indicate the percentage of sequencing reads with c.1412delA correction out of editable alleles that carry the mutation. Indel frequencies reflect all sequencing reads that contain any indels. Bars represent the mean of n=3 independent biological replicates. FIG. 46F shows prime editing in primary human T cells. Bars represent the mean of n=3 independent biological replicates from different healthy T cell donors.

FIGS. 47A-47J show the design and results of Repair-seq screens for substitution prime editing outcomes. FIG. 47A shows optimization of a Staphylococcus aureus (Sa)-pegRNA for installation of a G⋅C-to-C⋅G edit within a lentivirally integrated HBB sequence using SaPE2 in HEK293T cells. PBS, primer-binding site. Data represent the mean of n=3 independent biological replicates. FIG. 47B shows the design of the prime editing Repair-seq lentiviral vector (pPC1000). In Repair-seq screens, a 453-bp region containing CRISPRi sgRNA sequence and prime editing outcome is amplified from genomic DNA for paired-end Illumina sequencing. The CRISPRi sgRNA is sequenced with a 44-nt Illumina forward read (R1), and the prime edited site (including +50 and −50 nick sites) is sequenced with a 263-nt Illumina reverse read (R2). Black triangles indicate positions of SaPE2-induced nicks programmed by Sa-pegRNA and Sa-sgRNAs. Sizes of all vector components are to scale. FIG. 47C shows a schematic of PE2, PE3+50, and PE3-50 prime editing configurations with SaPE2 protein (SaCas9 N580A fused to an engineered MMLV RT). FIG. 47D shows validation of intended G⋅C-to-C⋅G editing at the lentivirally-integrated Repair-seq edit site in HeLa cells expressing dCas9-BFP-KRAB cells. Bars represent the mean of n=2 independent biological replicates. FIG. 47E shows prime editing at the Repair-seq edit site with and without blasticidin selection in HeLa cells expressing dCas9-BFP-KRAB. SaPE2-P2A-BlastR prime editor was used for all conditions. Bars represent the mean of n=2. FIG. 47F shows functional annotation classes of the genes targeted by the pooled CRISPRi sgRNA library used in Repair-seq screens. FIGS. 47G-47J show that the knockdown of MSH2, MSH6, MLH1, and PMS2 increases the frequency of the intended +6 G⋅C-to-C⋅G prime edit in all Repair-seq screens. Dots represent reads from individual CRISPRi sgRNAs.

FIGS. 48A-48I show the genetic modulators of unintended prime editing outcomes. FIG. 48A shows an overview of PE3-50 outcomes in HeLa CRISPRi screens. TP53BP1 knockdown dramatically reduces formation of all unintended editing outcomes. FIG. 48B shows additional details of PE2 outcomes in K562 CRISPRi screens, supplementing FIG. 41H. FIG. 48C shows additional details of PE3+50 outcomes in K562 CRISPRi screens, supplementing information in FIG. 41G. FIGS. 48D-48I show comparisons of effects of gene knockdown on frequencies of indicated outcome categories in indicated screen conditions. Plotted quantities are the mean of the log 2 fold changes from non-targeting sgRNAs for the two most extreme sgRNAs per gene, averaged over n=2 independent biological replicates per condition. Error bars mark the range of values spanned by the replicates. Black dots represent 20 random sets of three non-targeting sgRNAs. FIG. 48D shows that MSH2, MLH1, and PMS2 knockdown produce larger fold changes in installation of additional edits than in intended edits in K562 PE2 screens. FIG. 48E shows unintended joining of reverse transcribed sequence in PE2 screens in K562 and HeLa cells are most increased by knockdown of Fanconi anemia genes (red) as well as a set of RAD51 homologs and other genes involved in homologous recombination (blue). FIG. 48F shows deletions in in PE2 screens in K562 and HeLa cells are most increased by a set of RAD51 homologs and other genes involved in homologous recombination (blue). FIG. 48G shows that in addition to MSH2, MLH1, and PMS2, HLTF knockdown produces larger fold changes in installation of additional edits than in intended edits in K562 PE3+50 screens. FIG. 48H shows that tandem duplications in HeLa and K562 PE3+50 screens are most decreased by knockdown of POLD and RFC subunits. FIG. 48I shows deletions in HeLa PE3+50 and PE3-50 screens have dramatically divergent genetic regulators, highlighting differences in the processing of the different overhang configurations.

FIGS. 49A-49F show validation of prime editing Repair-seq screen results. FIGS. 49A-49B show alignment of Sa-pegRNAs, their templated 3′ DNA flaps following SaPE2 reverse transcription, and the genomic target sequence (top). Compared to the Sa-pegRNA used in Repair-seq screens (FIG. 49A), an Sa-pegRNA with recoded scaffold sequence (FIG. 49B) templates an extended 3′ DNA flap with reduced homology with genomic target sequence. The recoded Sa-pegRNA contains 2 base pair changes that preserve base pairing interactions within the scaffold. Reverse transcription of the Sa-pegRNA scaffold can generate a misextended 3′ flap that is incorporated into the genome. Vertical lines depict base pairing. X's depict mismatches between the misextended reverse-transcribed 3′ flap and genomic sequence. FIGS. 49A-49B also show frequencies of editing outcome categories observed at the screen edit site from arrayed PE2 and PE3+50 experiments in HeLa CRISPRi cells (bottom). Prime editing with the Sa-pegRNA used in siteRepair-seq screens (FIG. 49A) or a recoded Sa-pegRNA (FIG. 49B) results in different frequencies of installation of unintended edits from nearly-matched scaffold. Plotted quantities are the mean±SD of n=4 independent biological replicates, for each cell line containing MSH2 or non-targeting CRISPRi sgRNAs. FIG. 49C shows the mechanism of DNA mismatch repair in humans. FIG. 49D shows mismatch repair of a prime editing heteroduplex intermediate could install additional non-programmed nicks from MutLα endonuclease activity. Excision from these non-programmed nicks and subsequent repair of the resulting intermediates may contribute to larger and more frequent indel byproducts observed from MMR activity. FIG. 49E shows the knockdown efficiency of siRNA treatment relative to a non-targeting siRNA control in HEK293T cells. Cells were transfected with siRNAs, incubated for 3 days, transfected with PE2, pegRNAs, and the same siRNAs, then incubated for another 3 days before relative RNA abundances were assayed by RT-qPCR. NT, non-targeting. Data represent the mean of n=3 independent biological replicates. Each dot represents the mean of n=3 technical replicates. FIG. 49F shows editing in HEK293T cells co-transfected with prime editor components and siRNAs. Cells were not pre-treated with siRNAs before transfection with prime editor. Bars represent the mean of n=3 independent biological replicates.

FIGS. 50A-50H show the development and characterization of dominant negative MMR proteins that enhance prime editing. FIG. 50A shows the prime editing efficiencies from MMR proteins or dominant negative variants expressed in trans with or fused directly to PE2 in HEK293T cells. 32aa linker, (SGGS)×2-XTEN-(SGGS)×2 (SEQ ID NO: 125) (SGGSSGGSSGSETPGTSESATPES SGGSSGGS (SEQ ID NO: 125) or structurally. [SGGS]-[SGGS]-[SGSETPGTSESATPES]-[SGGS]-[SGGS](SEQ ID NO: 125)), codon opt., human codon optimized. Data within the same graph originate from experiments performed at the same time. Data represent the mean±SD of n=3 independent biological replicates. FIG. 50B shows titration of MLH1dn plasmid and PE2 plasmid transfection doses in HEK293T cells. Maximum plasmid amounts tested were 200 ng PE2 and 100 ng MLH1dn. Data represent the mean±SD of n=3 independent biological replicates. FIG. 50C shows prime editing with MLH1dn co-expression in MMR-deficient HCT116 cells that contain a biallelic deletion in MLH1. Bars represent the mean of 3 replicates. FIG. 50D shows a comparison of prime editing with human MLH1dn (human codon-optimized) or mouse MLH1dn (mouse codon optimized) in human HEK293T cells. Bars represent the mean of n=3 independent biological replicates. FIG. 50E shows a comparison of prime editing with human MLH1dn (human codon optimized) or mouse MLH1dn (mouse codon optimized) in mouse N2A cells. Bars represent the mean of n=3 replicates. FIG. 50F shows that MLH1 knockout in clonal HeLa cell lines enhances prime editing efficiency to a greater extent than ML H1 co-expression in clonal wild-type HeLa cells. A, knockout. Bars represent the mean of n=3 or 4 independent biological replicates. FIG. 50G shows editing at the FANCF locus with PE3b and PE5b (complementary-strand nick that is specific for the edited sequence) in HEK293T cells. PE5b, PE3b editing system with MLH1dn co-expression. Bars represent the mean of n=3 independent biological replicates. FIG. 50H shows editing at the HEK2 locus with complementary-strand nicks in HEK293T cells. “None” indicates the lack of a nick, which denotes a PE2 or PE4 editing strategy. Bars represent the mean of n=3 independent biological replicates.

FIGS. 51A-51J show the characterization of PE4 and PE5 across diverse prime edit classes and cell types. FIG. 51A shows a comparison of PE2, PE3, PE4, and PE5 for 84 single-base substitution prime edits across seven endogenous sites in HEK293T cells. Bars represent the mean of n=3 independent biological replicates). FIG. 51B provides a summary of PE4 enhancement in editing efficiency over PE2 for 84 single-base substitution edits across seven endogenous sites in HEK293T cells. PE4/PE2 fold improvements may be lower for PAM edits due to the high basal editing efficiency for PAM edits or the high representation of G⋅C-to-C⋅G edits (five out of 15 in this category). Data represent the mean±SD of n=3 independent biological replicates. FIG. 51C shows the efficiencies of single-base substitution prime edits that alter the PAM (+5 G and +6 G bases) of prime editing target protospacers in HEK293T cells. Four G⋅C-to-A⋅T, five G⋅C-to-C⋅G, and six G⋅C-to-T⋅A PAM edits across a combined seven endogenous sites are shown. The mean of all individual values of n=3 independent biological replicates are shown. FIG. 51D shows the effect of siRNA knockdown of MMR genes on G⋅C-to-C⋅G editing at the RNF2 locus in HEK293T cells. Bars represent the mean of n=3 independent biological replicates. FIG. 51E shows the effect of MMR gene knockout on G⋅C-to-C⋅G editing at the RNF2 locus in HAP1 cells. Δ, gene knockout. Bars represent the mean of n=3 independent biological replicates. FIG. 51F shows prime editing at the integrated screen edit site with CRISPRi knockdown in HeLa CRISPRi cells. PE2 indicates editing with SaPE2 protein and Sa-pegRNA. PE3+50 indicates editing with SaPE2 protein, Sa-pegRNA, and Sa-sgRNA that programs a +50 complementary-strand nick. Bars represent the mean of n=5 independent biological replicates. FIG. 51G provides a summary of PE5 enhancement in editing efficiency over PE3 for 84 single-base substitution edits in HEK293T cells. The grand mean±SD of all individual values of n=3 independent biological replicates are shown. FIG. 51H shows PE4 enhancement in editing efficiency over PE2 across a range of insertion and deletion prime edit lengths in HEK293T cells. A total of 33 different prime edits at a combined three endogenous loci are shown. The mean of all individual values of n=3 independent biological replicates are shown. FIG. 51I shows that PE5 improves editing efficiency and reduces indel byproducts compared to PE3 across small insertion and deletion prime edits in HEK293T cells. FIG. 51J shows PE2 and PE4 editing efficiencies at 33 different insertion and deletion prime edits across a combined three endogenous loci. Bars represent the mean of all individual values of n=3 independent biological replicates.

FIGS. 52A-52C show characterization of PE4 and PE5 systems and improved prime editing efficiency with additional silent mutations. FIG. 52A shows substitutions of contiguous bases with PE2 and PE4 in HEK293T cells. The top sequence indicates the original, unedited genomic sequence. Numbers denote the position of the edited nucleotide relative to the PE2 nick site. Nucleotides within the SpCas9 PAM sequence (NGG) are underlined. Sequences of the intended edited product are shown below, with edited nucleotides marked in red. Bars represent the mean of n=3 independent biological replicates. FIG. 52B shows that installation of additional silent mutations can increase prime editing efficiency by evading MMR. PE4/PE2 fold-change in editing frequency reflects the extent to which MMR activity impedes the indicated prime edit. Edited nucleotides that make the indicated coding mutation are marked in red, and edited nucleotides that make silent mutations are marked in green. Data represent the mean±SD of n=3 independent biological replicates. FIG. 52C shows installation of 22 single-base substitution prime edits across seven endogenous sites in HeLa cells with PE2, PE3, PE4, and PE5. Bars represent the mean of n=3 independent biological replicates.

FIGS. 53A-53G show the effect of dominant negative MLH1 on prime editing product purity and off-targeting. FIG. 53A shows the frequency of indels in HEK293T cells treated with pegRNAs, nicking sgRNAs, and PE2 enzyme, RT-impaired PE2 (PE2-dRT), or nickase Cas9 (SpCas9 H840A), with and without MLH1dn. Non-editing pegRNAs encode a 3′ DNA flap with perfect homology to the genomic target. Bars represent the mean of n=3 independent biological replicates. FIG. 53B shows the distribution of deletion outcomes from PE3 and PE5 at endogenous loci in HEK293T cells. 12 different pegRNAs that program single-base substitutions were tested at each indicated locus. Dotted lines indicate position of pegRNA- and sgRNA-directed nicks. Data represent the mean±SD of n=3 independent biological replicates. FIG. 53C shows the distribution of deletion outcomes from PE3 and PE5 with an edit-encoding and non-editing pegRNA in HEK293T cells. The non-editing pegRNA templates a 3′ DNA flap with perfect complementarity to the genomic target sequence. Data represent the mean±SD of n=3 independent biological replicates. FIG. 53D shows the frequency of all prime editing outcomes with unintended pegRNA scaffold sequence incorporation or unintended flap rejoining in HEK293T cells. 12 pegRNAs each programming a different single-base substitution were tested at each of the seven indicated loci. Each dot represents an individual pegRNA at the indicated loci (mean of n=3 independent biological replicates). FIG. 53E shows the off-target prime editing by PE2 and PE4 in HEK293T cells (mean of n=3 independent biological replicates). FIG. 53F shows the distribution and cumulative distribution of microsatellite repeat lengths in the indicated cell types and treatments. HAP1 and HeLa cells are MMR-proficient, and HCT116 cells have impaired MMR. HAP1 ΔMSH2 cells underwent 60 cell divisions following knockout of MSH2. HeLa cells were transiently transfected with PE2 or PE4 components and grown for a following 3 days before sequencing, wt, wild-type. All values from n=2 independent biological replicates are shown. FIG. 53G shows prime editing at the on-target locus in HeLa cells transfected with PE2 or PE4 components. Bars represent the mean of n=2 independent biological replicates. Microsatellite lengths were assayed from genomic DNA taken from these PE2 and PE4-treated HeLa cells.

FIGS. 54A-54F show that use of PEmax architecture with PE4 and PE5 editing systems enhances editing at disease-relevant gene targets and cell types. FIG. 54A shows a schematic of PE2 and PEmax editor architectures. bpNLS^{SV40}, bipartite SV40 NLS. MMLV RT, Moloney Murine Leukemia Virus reverse transcriptase pentamutant. GS codon, Genscript human codon optimized. FIG. 54B shows engineered pegRNAs (epegRNAs) containing a 3′ RNA structural motif that improve prime editing performance. FIG. 54C shows prime editing efficiencies of PE4 and PE5 combined with PEmax architectures and epegRNAs. Seven single-base substitution edits targeting different loci were tested in HeLa and HEK293T cells. Fold changes indicate the average of fold increases from each edit tested. The mean±SD of all individual values of n=3 independent biological replicates are shown. FIG. 54D shows prime editing at therapeutically-relevant sites in wild-type HeLa and HEK293T cells. The HBB locus is edited at the E6 codon commonly mutated in patients with sickle cell disease (E6V). The CDKL5 edit is at a site for which the c.1412delA mutation causes CDKL5 deficiency disorder. epegRNAs were used for editing the HBB, PRNP, and CDKL5 loci. Bars represent the mean of n=3 independent biological replicates. FIG. 54E shows the correction of CDKL5 c.1412delA via an A⋅T insertion and a silent G⋅C-to-A⋅T edit in iPSCs derived from a patient heterozygous for the allele. Editing efficiencies indicate the percentage of sequencing reads with c.1412delA correction out of editable alleles that carry the mutation. Indel frequencies reflect all sequencing reads that contain any indels. Bars represent the mean of n=3 independent biological replicates. FIG. 54F shows prime editing in primary T cells. Bars represent the mean of n=3 independent biological replicates from different healthy T cell donors.

FIGS. 55A-55B show the development of PEmax and application of PE4 and PE5 to primary cell types. FIGS. 55A-55B show screening of prime editor variants to maximize editing efficiency in HeLa cells. All prime editor architectures carry a Cas9 H840A mutation to prevent nicking of the complementary DNA strand at the target protospacer. *NLSSV40 contains a 1-aa deletion outside the PKKKRKV (SEQ ID NO: 132) NLSSV40 consensus sequence. All individual values of n=3 independent biological replicates are shown.

FIGS. 56A-56G show development of PEmax and application of PE4 and PE5 to primary cell types. FIG. 56A shows a screen of prime editor variants for improved editing efficiency with the PE3 system in HeLa cells. All prime editor architectures carry a SpCas9 H840A mutation to prevent nicking of the complementary DNA strand at the target protospacer. NLSSV40 indicates the bipartite SV40 NLS. *NLSSV40 contains a 1-aa deletion outside the PKKKRKV (SEQ ID NO: 132) NLSSV40 consensus sequence. All individual values of n=3 independent biological replicates are shown. FIG. 56B shows the architecture of the original PE2 editor (Anzalone et al., 2019), PE2* (Liu et al., 2021). CM P-PE-V1 (Park et al., 2021), and prime editor variants developed in this work (PEmax, CMP-PEmax). HN1, HMGN1; H1G, histone H1 central globular domain; codon opt., human codon optimized. FIG. 56C shows that PEmax outperforms other prime editor architectures with the PE3 system in HeLa cells. Bars represent the mean of n=3 independent biological replicates. FIG. 56D shows fold-change in editing efficiency of prime editor architectures compared to PE2 with the PE3 system in HeLa cells. The mean±SD of all individual values of n=3 independent biological replicates are shown. FIG. 56E shows the intended editing and indel frequencies from PE4, PE4max (PE4 editing system with PEmax architecture), PE5, and PE5max (PE5 editing system with PEmax architecture) in HeLa and HEK293T cells. Seven substitution prime edits targeting different endogenous loci were tested for each condition. The mean±SD of all individual values of n=3 independent biological replicates are shown. FIG. 56F shows the correction of CDKL5 c.1412delA via an A⋅T insertion and a G⋅C-to-A⋅T edit in iPSCs derived from a patient heterozygous for the disease allele. Editing efficiencies indicate the percentage of sequencing reads with c.1412delA correction out of editable alleles that carry the mutation. Indel frequencies reflect all sequencing reads that contain any indels that do not map to the c.1412delA allele or wild-type sequence. 1 μg of PE2 mRNA was used in all conditions shown. Bars represent the mean of n=3 independent biological replicates. FIG. 56G shows prime editing in primary T cells. Bars represent the mean of n=3 independent replicates from different T cell donors.

FIGS. 57A-57B show that the recoded pegRNA scaffold reduces unintended outcomes from scaffold sequence incorporation. FIG. 57A shows an alignment of the prime editing Repair-seq target site and SaPE2-generated 3′ DNA flaps templated by (top) the Sa-pegRNA used in Repair-seq screens, or (bottom) an Sa-pegRNA with a recoded scaffold sequence. 3′ flap sequences are aligned with the templated region of the Sa-pegRNA shown above (RT template or scaffold). Red indicates position of the intended +6 G⋅C to C⋅G edit programmed by both Sa-pegRNAs. Blue indicates positions at which the genomic target sequence does not align with the 3′ flap sequence templated by the Sa-pegRNA scaffold. Unintended edits from incorporation of a 3′ flap containing a reverse transcribed Sa-pegRNA scaffold sequence may occur at these blue-indicated nucleotides. FIG. 57B shows a summary of editing outcome categories observed in PE2 and PE3+50 experiments in HeLa CRISPRi cells. Screen pegRNA indicates the Sa-pegRNA used in prime editing Repair-seq screens. Sa-pegRNA with recoded scaffold (sequence shown in FIG. 54A) avoids sequence homology with the Repair-seq edit site. Plotted quantities are the mean±SD of one CRISPRi sgRNA for each indicated target (MSH2 and non-targeting), averaged across n=4 independent biological replicates.

FIG. 58 shows a comparison of PE3max (PE3 editing system with PEmax protein) and PE3 (PE3 editing system with PE2 protein) in HeLa cells (mean of n=3 independent biological replicates).

FIG. 59 shows that PE improvement with MLH1dn depends on prime edit size. MMR most efficiently repairs substitutions and insertion and deletion errors of fewer than or equal to approximately 13 bp in length.

FIG. 60 shows that PE4 and epegRNAs enable prime editing with a single pegRNA integrant.

FIG. 61 shows that PE5 improves installation of the protective Christchurch allele in an APOE4 mouse astrocyte model.

FIGS. 62A-62C show that inhibiting p53 enhances the efficiency and precision of PE3 prime editing. This is particularly true when the nicking sgRNA makes a nick upstream (− side) of the pegRNA-directed nick. Each point on the graphs represents an individual CRISPRi gene knockdown in the Repair-seq screens. The axes depict log 2 fold changes compared to control. Knocking down TP53BP1 (p53 gene) increases intended editing (x-axis) and decreases three types of unintended editing (y-axes), including joining of the reverse transcribed sequence at unintended locations (FIG. 62A), unintended deletions (FIG. 62B), and unintended tandem duplications (FIG. 62C).

FIG. 63 shows that a p53 inhibitor (i53) can enhance the efficiency and precision of PE3 prime editing. This is particularly true when the nicking sgRNA makes a nick upstream (− side) of the pegRNA-directed nick. Only the EMX1 site uses a nick on the “−” side. FIG. 64 represents various aspects of the disclosure, including the use of CRISPRi screens to reveal cellular genes—including mismatch repair genes—having an impact on prime editing outcomes, the use of engineered MLH1 of the mismatch repair (MMR) pathway to enhance the efficiency and precision of prime editing, and the demonstration that improved prime editing systems described herein (e.g., PE4 and PE5 systems, and PEmax editor) were shown to exhibit the same beneficial effects in many cell types.

FIG. 64 shows that CRISPRi screens reveal cellular determinants of prime genome editing, that engineered MLH1 protein enhances prime editing efficiency and precision, and that improved prime editing systems were characterized across edit and cell types.

FIG. 65 provides a schematic showing the optimization of PE2 protein.

FIG. 66 shows the fold change in the frequency of the intended edit using PE2 and various other PE constructs in HEK293T cells (low plasmid dose) at a range of gene targets (HEK3, EMX1, RNF2, FANCF, FUNX1, DNMT1. VEGFA, HEK4, PRNP, APOE, CXCR4, HEK3).

FIG. 67 shows the fold change in the frequency of the intended edit using PE3 and various prime editor constructs in HeLa cells at a range of gene targets (HEK3, FANCF, RUNX1, VEGFA).

FIG. 68 shows a comparison of prime editing in HEK293T vs. HeLa editing using various PE constructs.

FIG. 69 shows NLS architecture optimization of PE3 in HeLa cells.

FIG. 70 provides a schematic showing the final PEmax construct, which corresponds to SEQ ID NO: 99.

FIG. 71 shows that PEmax increases indels in addition to the intended edit.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)—associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc), and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which are hereby incorporated by reference, Cas9 recognizes a short motif in the CRISPR repeat sequences (the PAM or protospacer adjacent motif) to help distinguish self versus non-self, Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference), Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science, 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 2). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 2). In some embodiments, the Cas9 variant comprises a fragment of SEQ ID NO: 2 Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 2). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 2).

### Circular Permutant

As used herein, the term “circular permutant” refers to a protein or polypeptide (e.g., a Cas9) comprising a circular permutation, which is a change in the protein's structural configuration involving a change in the order of amino acids appearing in the protein's amino acid sequence. In other words, circular permutants are proteins that have altered N- and C-termini as compared to a wild-type counterpart, e.g., the wild-type C-terminal half of a protein becomes the new N-terminal half. Circular permutation (or CP) is essentially the topological rearrangement of a protein's primary sequence, connecting its N- and C-terminus, often with a peptide linker, while concurrently splitting its sequence at a different position to create new, adjacent N- and C-termini. The result is a protein structure with different connectivity, but which often can have the same overall similar three-dimensional (3D) shape, and possibly include improved or altered characteristics, including reduced proteolytic susceptibility, improved catalytic activity, altered substrate or ligand binding, and/or improved thermostability. Circular permutant proteins can occur in nature (e.g., concanavalin A and lectin). In addition, circular permutation can occur as a result of posttranslational modifications or may be engineered using recombinant techniques.

### Circularly Permuted Cas9

The term “circularly permuted Cas9” refers to any Cas9 protein, or variant thereof, that occurs as a circular permutant, whereby its N- and C-termini have been topically rearranged. Such circularly permuted Cas9 proteins (“CP-Cas9”), or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of which are incorporated herein by reference. The instant disclosure contemplates any previously known CP-Cas9 or use of a new CP-Cas9 so long as the resulting circularly permuted protein retains the ability to bind DNA when complexed with a guide RNA (gRNA). Exemplary CP-Cas9 proteins are SEQ ID NOs: 54-63.

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821(2012), the entire contents of which is hereby incorporated by reference, Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In certain types of CRISPR systems (e.g., type II CRISPR systems), correct processing of pre-crRNA requires a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves a linear or circular nucleic acid target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered to incorporate embodiments of both the crRNA and tracrRNA into a single RNA species—the guide RNA.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. The tracrRNA of the system is complementary (fully or partially) to the tracr mate sequence present on the guide RNA.

### DNA Synthesis Template

As used herein, the term “DNA synthesis template” refers to the region or portion of the extension arm of a PEgRNA that is utilized as a template strand by a polymerase of a prime editor to encode a 3′ single-strand DNA flap that contains the desired edit and which then, through the mechanism of prime editing, replaces the corresponding endogenous strand of DNA at the target site. The extension arm, including the DNA synthesis template, may be comprised of DNA or RNA. In the case of RNA, the polymerase of the prime editor can be an RNA-dependent DNA polymerase (e.g., a reverse transcriptase). In the case of DNA, the polymerase of the prime editor can be a DNA-dependent DNA polymerase. In various embodiments the DNA synthesis template may comprise the “edit template” and the “homology arm”, and all or a portion of the optional 5′ end modifier region, e2. That is, depending on the nature of the e2 region (e.g., whether it includes a hairpin, toeloop, or stem/loop secondary structure), the polymerase may encode none, some, or all of the e2 region as well. Said another way, in the case of a 3′ extension arm, the DNA synthesis template can include the portion of the extension arm that spans from the 5′ end of the primer binding site (PBS) to 3′ end of the gRNA core that may operate as a template for the synthesis of a single-strand of DNA by a polymerase (e.g., a reverse transcriptase). In the case of a 5′ extension arm, the DNA synthesis template can include the portion of the extension arm that spans from the 5′ end of the PEgRNA molecule to the 3′ end of the edit template. In some embodiments, the DNA synthesis template excludes the primer binding site (PBS) of PEgRNAs either having a 3′ extension arm or a 5′ extension arm. Certain embodiments described here refer to an “an RT template,” which is inclusive of the edit template and the homology arm, i.e., the sequence of the PEgRNA extension arm which is actually used as a template during DNA synthesis. The term “RT template” is equivalent to the term “DNA synthesis template.” in certain embodiments, an RT template may be used to refer to a template polynucleotide for reverse transcription, e.g., in a prime editing system, complex, or method using a prime editor having a polymerase that is a reverse transcriptase. In some embodiments, a DNA synthesis template may be used to refer to a template polynucleotide for DNA polymerization, e.g., RNA-dependent DNA polymerization or DNA-dependent DNA polymerization, e.g., in a prime editing system, complex, or method using a prime editor having a polymerase that is an RNA-dependent DNA polymerase or a DNA-dependent DNA polymerase.

In some embodiments, the DNA synthesis template is a single-stranded portion of the PEgRNA that is 5′ of the PBS and comprises a region of complementarity to the PAM strand (i.e., the non-target strand or the edit strand), and comprises one or more nucleotide edits compared to the endogenous sequence of the double stranded target DNA. In some embodiments, the DNA synthesis template is complementary or substantially complementary to a sequence on the non-target strand that is downstream of a nick site, except for one or more non-complementary nucleotides at the intended nucleotide edit positions. In some embodiments, the DNA synthesis template is complementary or substantially complementary to a sequence on the non-target strand that is immediately downstream (i.e., directly downstream) of a nick site, except for one or more non-complementary nucleotides at the intended nucleotide edit positions. In some embodiments, one or more of the non-complementary nucleotides at the intended nucleotide edit positions are immediately downstream of a nick site. In some embodiments, the DNA synthesis template comprises one or more nucleotide edits relative to the double-stranded target DNA sequence. In some embodiments, the DNA synthesis template comprises one or more nucleotide edits relative to the non-target strand of the double-stranded target DNA sequence. For each PEgRNA described herein, a nick site is characteristic of the particular napDNAbp to which the gRNA core of the PEgRNA associates, and is characteristic of the particular PAM required for recognition and function of the napDNAbp. For example, for a PEgRNA that comprises a gRNA core that associates with a SpCas9, the nick site in the phosphodiester bond between bases three (“−3” position relative to the position 1 of the PAM sequence) and four (“−4” position relative to position 1 of the PAM sequence). In some embodiments, the DNA synthesis template and the primer binding site are immediately adjacent to each other. The terms “nucleotide edit”, “nucleotide change”, “desired nucleotide change”, and “desired nucleotide edit” are used interchangeably to refer to a specific nucleotide edit, e.g., a specific deletion of one or more nucleotides, a specific insertion of one or more nucleotides, a specific substitution (or multiple substitutions) of one or more nucleotides, or a combination thereof, at a specific position in a DNA synthesis template of a PEgRNA to be incorporated in a target DNA sequence. In some embodiments, the DNA synthesis template comprises more than one nucleotide edit relative to the double-stranded target DNA sequence. In such embodiments, each nucleotide edit is a specific nucleotide edit at a specific position in the DNA synthesis template, each nucleotide edit is at a different specific position relative to any of the other nucleotide edits in the DNA synthesis template, and each nucleotide edit is independently selected from a specific deletion of one or more nucleotides, a specific insertion of one or more nucleotides, a specific substitution (or multiple substitutions) of one or more nucleotides, or a combination thereof. A nucleotide edit may refer to the edit on the DNA synthesis template as compared to the sequence on the target strand of the target gene, or a nucleotide edit may refer to the edit encoded by the DNA synthesis template on the newly synthesized single stranded DNA that replaces the endogenous target DNA sequence on the non-target strand.

### Dominant Negative Variant

The terms “dominant negative variant” and “dominant negative mutant” refer to genes or gene products (e.g., proteins) that comprise a mutation that results in the gene product acting antagonistically to the wild-type gene product (i.e., inhibiting its activity). Dominant negative mutations generally result in an altered molecular function (often inactive). For example, the present disclosure provides dominant negative variants of MMR proteins that inhibit the activity of wild-type MMR proteins (e.g., the dominant negative MLH1 proteins described herein).

### Edit Template

The term “edit template” refers to a portion of the extension arm that encodes the desired edit in the single strand 3′ DNA flap that is synthesized by the polymerase, e.g., a DNA-dependent DNA polymerase, RNA-dependent DNA polymerase (e.g., a reverse transcriptase). Certain embodiments described here refer to “an RT template,” which refers to both the edit template and the homology arm together, i.e., the sequence of the PEgRNA extension arm which is actually used as a template during DNA synthesis. The term “RT edit template” is also equivalent to the term “DNA synthesis template,” but wherein the RT edit template reflects the use of a prime editor having a polymerase that is a reverse transcriptase, and wherein the DNA synthesis template reflects more broadly the use of a prime editor having any polymerase.

### Extension Arm

The term “extension arm” refers to a nucleotide sequence component of a PEgRNA which comprises a primer binding site and a DNA synthesis template (e.g., an edit template and a homology arm) for a polymerase (e.g., a reverse transcriptase). In some embodiments, the extension arm is located at the 3′ end of the guide RNA. In other embodiments, the extension arm is located at the 5′ end of the guide RNA. In some embodiments, the extension arm comprises a DNA synthesis template and a primer binding site. In some embodiments, the extension arm comprises the following components in a 5′ to 3′ direction: the DNA synthesis template and the primer binding site. In some embodiments, the extension arm also includes a homology arm. In various embodiments, the extension arm comprises the following components in a 5′ to 3′ direction: the homology arm, the edit template, and the primer binding site. Since polymerization activity of the reverse transcriptase is in the 5′ to 3′ direction, the preferred arrangement of the homology arm, edit template, and primer binding site is in the 5′ to 3′ direction such that the reverse transcriptase, once primed by an annealed primer sequence, polymerizes a single strand of DNA using the edit template as a complementary template strand.

The extension arm may also be described as comprising generally two regions: a primer binding site (PBS) and a DNA synthesis template, for instance. The primer binding site binds to the primer sequence that is formed from the endogenous DNA strand of the target site when it becomes nicked by the prime editor complex, thereby exposing a 3′ end on the endogenous nicked strand. As explained herein, the binding of the primer sequence to the primer binding site on the extension arm of the PEgRNA creates a duplex region with an exposed 3′ end (i.e., the 3′ of the primer sequence), which then provides a substrate for a polymerase to begin polymerizing a single strand of DNA from the exposed 3′ end along the length of the DNA synthesis template. The sequence of the single strand DNA product is the complement of the DNA synthesis template. Polymerization continues towards the 5′ of the DNA synthesis template (or extension arm) until polymerization terminates. Thus, the DNA synthesis template represents the portion of the extension arm that is encoded into a single strand DNA product (i.e., the 3′ single strand DNA flap containing the desired genetic edit information) by the polymerase of the prime editor complex and which ultimately replaces the corresponding endogenous DNA strand of the target site that sits immediately downstream of the PE-induced nick site. Without being bound by theory, polymerization of the DNA synthesis template continues towards the 5′ end of the extension arm until a termination event. Polymerization may terminate in a variety of ways, including, but not limited to (a) reaching a 5′ terminus of the PEgRNA (e.g., in the case of the 5′ extension arm wherein the DNA polymerase simply runs out of template), (b) reaching an impassable RNA secondary structure (e.g., hairpin or stem/loop), or (c) reaching a replication termination signal, e.g., a specific nucleotide sequence that blocks or inhibits the polymerase, or a nucleic acid topological signal, such as, supercoiled DNA or RNA.

### Fusion Protein

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes a Cas9 or equivalent thereof to a reverse transcriptase. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

Guide RNA (“gRNA”)

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to the protospacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein. In addition, methods for designing appropriate guide RNA sequences are provided herein. As used herein, the “guide RNA” may also be referred to as a “traditional guide RNA” to contrast it with the modified forms of guide RNA termed “prime editing guide RNAs” (or “PEgRNAs”).

Guide RNAs or PEgRNAs may comprise various structural elements that include, but are not limited to:

Spacer sequence—the sequence in the guide RNA or PEgRNA (having about 20 nts in length) which binds to the protospacer in the target DNA.

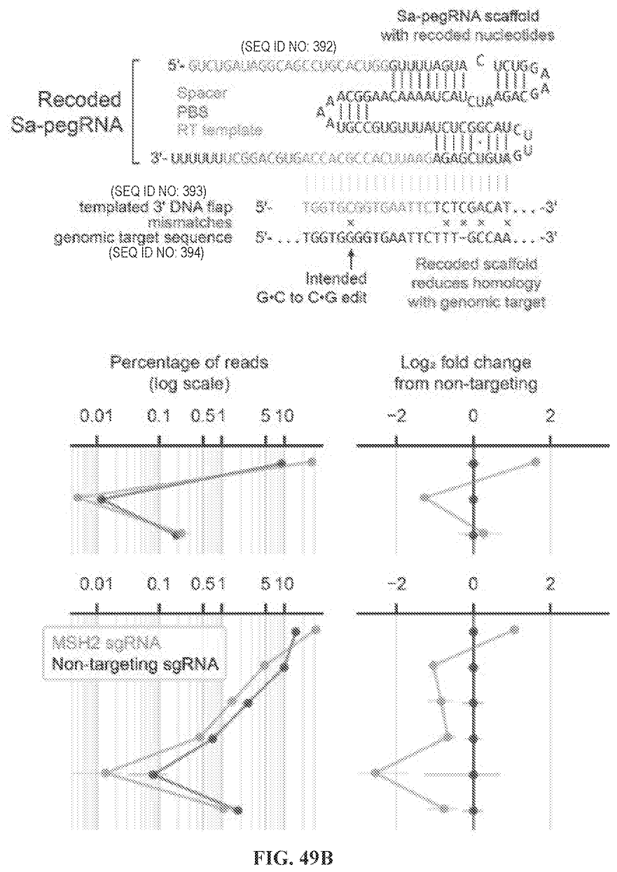

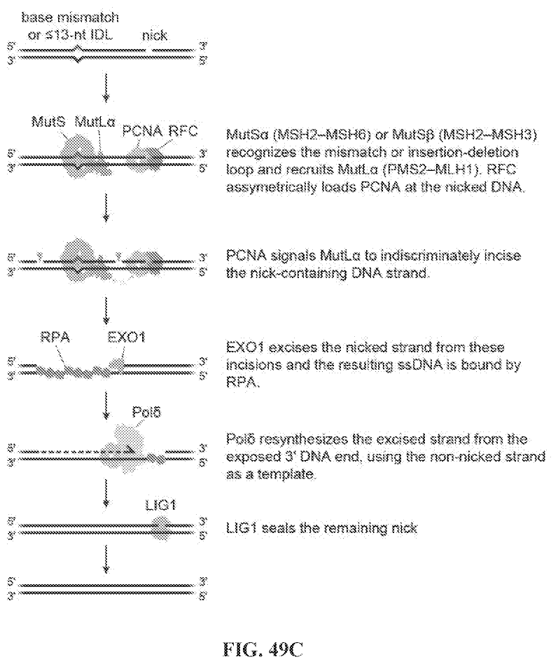

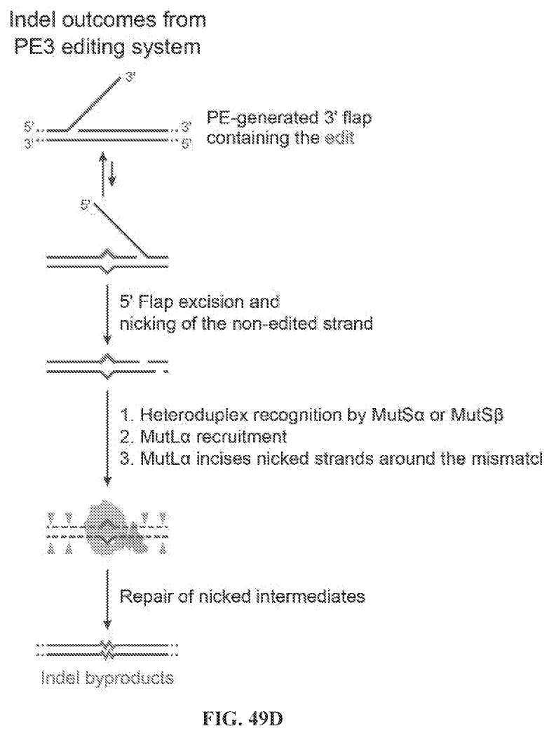

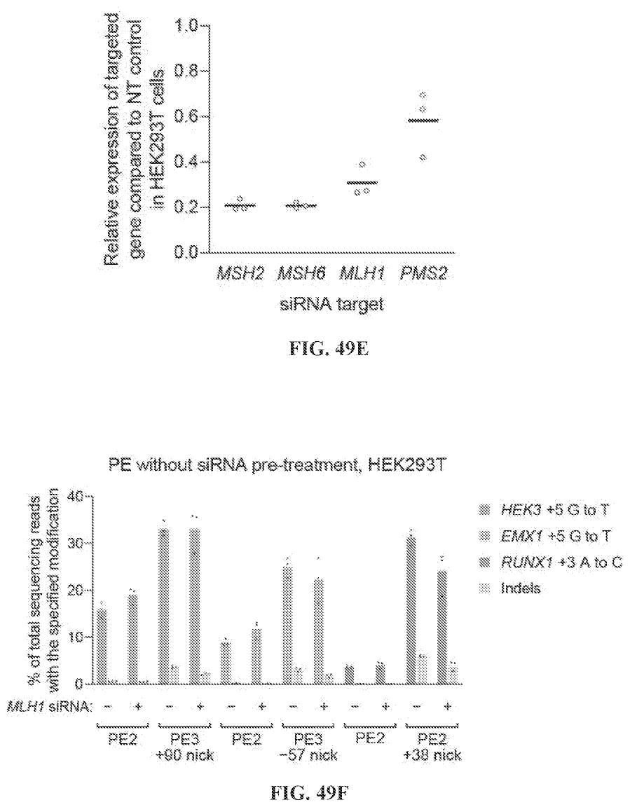

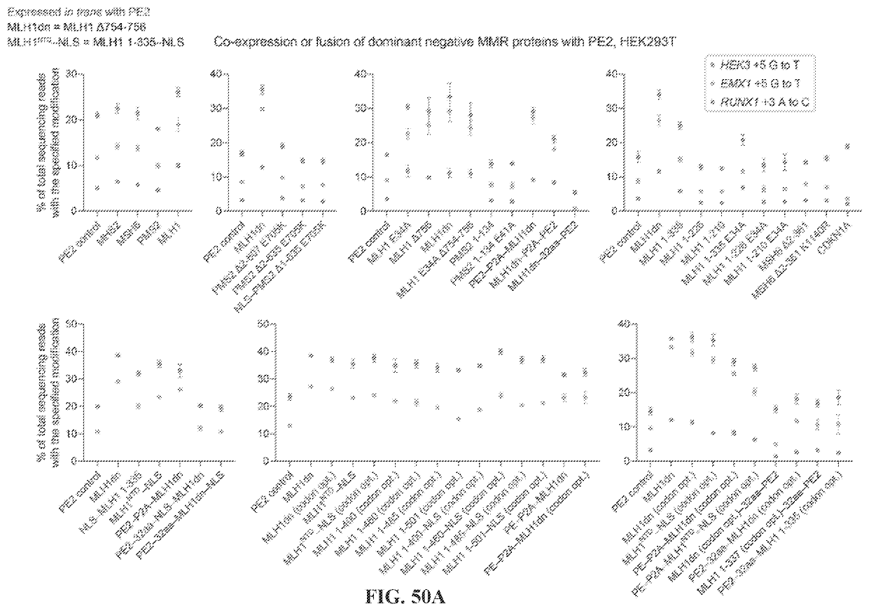

gRNA core (or gRNA scaffold or backbone sequence)—refers to the sequence within the gRNA that is responsible for Cas9 binding, it does not include the 20 bp spacer/targeting sequence that is used to guide Cas9 to target DNA. In some embodiments, the gRNA core or scaffold comprises a sequence that comprises one or more nucleotide alterations compared to a naturally occurring CRISPR-Cas guide RNA scaffold, for example, a Cas9 guide RNA scaffold. In some embodiments, the sequence of the gRNA core is designed to comprise minimal or no sequence homology to the endogenous sequence of the target nucleic acid at the target site, thereby reducing unintended edits. For example, in some embodiments, one or more base pairs in the second stem loop of a Cas9 gRNA core may be “flipped” (e.g., the G-U base pair and the U-A base pair as exemplified in FIG. 49A) to reduce unintended edits. In some embodiments, the gRNA core comprises no more than 1%, 5%, 10%, 15%, 20%, 25%, or 30% sequence homology to the sequence of the double stranded target DNA that flanks 5, 10, 15, 20, 25, 30, 35, 40, 45, or 50 nucleotides upstream or downstream of the position of the one or more nucleotide edits

Extension arm—a single strand extension at the 3′ end or the 5′ end of the PEgRNA which comprises a primer binding site and a DNA synthesis template sequence that encodes via a polymerase (e.g., a reverse transcriptase) a single stranded DNA flap containing the genetic change of interest, which then integrates into the endogenous DNA by replacing the corresponding endogenous strand, thereby installing the desired genetic change.

Transcription terminator—the guide RNA or PEgRNA may comprise a transcriptional termination sequence at the 3′ of the molecule. In some embodiments, the PEgRNA comprises a transcriptional termination sequence between the DNA synthesis template and the gRNA core.

### Homology

The terms “homologous,” “homology,” or “percent homology” as used herein refer to the degree of sequence identity between an amino acid or polynucleotide sequence and a corresponding reference sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95%, or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer or protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide, or portion thereof. In some embodiments, the homology or identity is assessed over a functional portion or a specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman. “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.ukiTools/psa/emboss_needle/) which is part of the EMBOSS package (Rice P et al., Trends Genet., 2000; 16: 276-277), and the GGSEARCH program fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm, which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998).

A skilled person understands that amino acid (or nucleotide) positions may be determined in homologous sequences based on alignment. For example, “H840” in a reference Cas9 sequence may correspond to H839, or another corresponding position in a Cas9 homolog when aligned to the reference Cas9 sequence.

### Host Cell

The term “host cell,” as used herein, refers to a cell that can host, replicate, and express a vector described herein, e.g., a vector comprising a nucleic acid molecule encoding an MLH1 variant and a fusion protein comprising a Cas9 or Cas9 equivalent and a reverse transcriptase.

### Inhibit

As used herein the term “inhibiting,” “inhibit,” or “inhibition” in the context of proteins and enzymes, for example, in the context of enzymes involved in the DNA mismatch repair pathway, refers to a reduction in the activity of the protein or enzyme. In some embodiments, the term refers to a reduction of the level of enzyme activity, e.g., the activity of one or more enzymes in the DNA mismatch repair pathway, to a level that is statistically significantly lower than an initial level, which may, for example, be a baseline level of enzyme activity. In some embodiments, the term refers to a reduction of the level of enzyme activity, e.g., the activity of one or more enzymes in the DNA mismatch repair pathway, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

### Linker

The term “linker,” as used herein, refers to a molecule linking two other molecules or moieties. The linker can be an amino acid sequence in the case of a linker joining two fusion proteins. For example, a Cas9 can be fused to a reverse transcriptase by an amino acid linker sequence. The linker can also be a nucleotide sequence in the case of joining two nucleotide sequences together. For example, in the instant case, the traditional guide RNA is linked via a spacer or linker nucleotide sequence to the RNA extension of a prime editing guide RNA which may comprise a RT template sequence and an RT primer binding site. In other embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In certain embodiments, the linker is a self-hydrolyzing linker (e.g., a 2A self-cleaving peptide as described further herein). Self-hydrolyzing linkers such as 2A self-cleaving peptides are capable of inducing ribosomal skipping during protein translation, resulting in the ribosome failing to make a peptide bond between two genes, or gene fragments.

### MLH1

The term “MLH1” refers to a gene encoding MLH1 (or MutL Homolog 1), a DNA mismatch repair enzyme. The protein encoded by this gene can heterodimerize with mismatch repair endonuclease PMS2 to form MutL alpha (MutLα), part of the DNA mismatch repair system. MLH1 mediates protein-protein interactions during mismatch recognition, strand discrimination, and strand removal. In mismatch repair, the heterodimer MSH2:MSH6 (MutSα) forms and binds the mismatch. MLH1 then forms a heterodimer with PMS2 (MutLα) and binds the MSH2:MSH6 heterodimer. The MutLα heterodimer then incises the nicked strand 5′ and 3′ of the mismatch, followed by excision of the mismatch from MutLα-generated nicks by EXO1. Finally, POLδ resynthesizes the excised strand, followed by LIG1 ligation.

An exemplary amino acid sequence of MLH1 is human isoform 1, P40692-1:

>sp|P40692|MLH1_HUMAN DNA mismatch repair protein Mlh1 OS = Homo sapiens OX = 9606
GN = MLH1 PE = 1 SV = 1:
(SEQ ID NO: 204)
MSFVAGVIRRLDETVVNRIAAGEVIQRPANAIKEMIENCLDAKSTSIQVIVKEGGLKLIQIQDNGTGIRKEDL
DIVCERFTTSKLQSFEDLASISTYGFRGEALASISHVAHVTITTKTADGKCAYRASYSDGKLKAPPKPCAGN
QGTQITVEDLFYNIATRRKALKNPSEEYGKILEVVGRYSVHNAGISFSVKKQGETVADVRTLPNASTVDNIR
SIFGNAVSRELIEIGCEDKTLAFKMNGYISNANYSVKKCIFLLFINHRLVESTSLRKAIETVYAAYLPKNTHPF
LYLSLEISPQNVDVNVHPTKHEVHFLHEESILERVQQHIESKLLGSNSSRMYFTQTLLPGLAGPSGEMVKST
TSLTSSSTSGSSDKVYAHQMVRTDSREQKLDAFLQPLSKPLSSQPQAIVTEDKTDISSGRARQQDEEMLELP
APAEVAAKNQSLEGDTTKGTSEMSEKRGPTSSNPRKRHREDSDVEMVEDDSRKEMTAACTPRRRIINLTSV
LSLQEEINEQGHEVLREMLHNHSFVGCVNPQWALAQHQTKLYLLNTTKLSEELFYQILIYDFANFGVLRLS
EPAPLFDLAMLALDSPESGWTEEDGPKEGLAEYIVEFLKKKAEMLADYFSLEIDEEGNLIGLPLLIDNYVPPL
EGLPIFILRLATEVNWDEEKECFESLSKECAMFYSIRKQYISEESTLSGQQSEVPGSIPNSWKWTVEHIVYKA
LRSHILPPKHFTEDGNILQLANLPDLYKVFERC,

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 204.

Another exemplary amino acid sequence of MLH1 is human isoform 2, P40692-2 (wherein amino acids 1-241 of isoform 1 are missing): >sp|P40692-2|MLH1_HUMAN Isoform 2 of DNA mismatch repair protein Mlh 1 OS═Homo sapiens OX=9606 GN=MLH1:

(SEQ ID NO: 205)
MNGYISNANYSVKKCIFLLFINHRLVESTSLRKAIETVYAAYLPKNTHPFLYLSLEISPQNVDVNVHPTKHE
VHFLHEESILERVQQHIESKLLGSNSSRMYFTQTLLPGLAGPSGEMVKSTTSLTSSSTSGSSDKVYAHQMVR
TDSREQKLDAFLQPLSKPLSSQPQAIVTEDKTDISSGRARQQDEEMLELPAPAEVAAKNQSLEGDTTKGTSE
MSEKRGPTSSNPRKRHREDSDVEMVEDDSRKEMTAACTPRRRIINLTSVLSLQEEINEQGHEVLREMLHNH
SFVGCVNPQWALAQHQTKLYLLNTTKLSEELFYQILIYDFANFGVLRLSEPAPLFDLAMLALDSPESGWTE
EDGPKEGLAEYIVEFLKKKAEMLADYFSLEIDEEGNLIGLPLLIDNYVPPLEGLPIFILRLATEVNWDEEKEC
FESLSKECAMFYSIRKQYISEESTLSGQQSEVPGSIPNSWKWTVEHIVYKALRSHILPPKHFTEDGNILQLAN
LPDLYKVFERC,

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 205.

Another exemplary amino acid sequence of MLH1 is human isoform 3, P40692-3 (where amino acids 1-101 (MSFVAGVIRR . . . ASISTYGFRG (SEQ ID NO: 206) is replaced with MAF): >sp|P40692-2|MLH1_HUMAN Isoform 2 of DNA mismatch repair protein Mlh1 OS═Homo sapiens OX=9606 GN=MLH1:

(SEQ ID NO: 207)
MAFEALASISHVAHVTITTKTADGKCAYRASYSDGKLKAPPKPCAGNQGTQITVEDLFYNIATRRKALKNP
SEEYGKILEVVGRYSVHNAGISFSVKKQGETVADVRTLPNASTVDNIRSIFGNAVSRELIEIGCEDKTLAFKM
NGYISNANYSVKKCIFLLFINHRLVESTSLRKAIETVYAAYLPKNTHPFLYLSLEISPQNVDVNVHPTKHEVH
FLHEESILERVQQHIESKLLGSNSSRMYFTQTLLPGLAGPSGEMVKSTTSLTSSSTSGSSDKVYAHQMVRTD
SREQKLDAFLQPLSKPLSSQPQAIVTEDKTDISSGRARQQDEEMLELPAPAEVAAKNQSLEGDTTKGTSEMS
EKRGPTSSNPRKRHREDSDVEMVEDDSRKEMTAACTPRRRIINLTSVLSLQEEINEQGHEVLREMLHNHSF
VGCVNPQWALAQHQTKLYLLNTTKLSEELFYQILIYDFANFGVLRLSEPAPLFDLAMLALDSPESGWTEED
GPKEGLAEYIVEFLKKKAEMLADYFSLEIDEEGNLIGLPLLIDNYVPPLEGLPIFILRLATEVNWDEEKECFES
LSKECAMFYSIRKQYISEESTLSGQQSEVPGSIPNSWKWTVEHIVYKALRSHILPPKHFTEDGNILQLANLPD
LYKVFERC,

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 207.

The present disclosure contemplates targeting MLH1 and/or MMR pathway components that interact with MLH1, including any wildtype or naturally occurring variant of MLH1, including any amino acid sequence having at least 70%, or 75%, or 80%, or 85%, or 90%, or 95%, or 99% or more sequence identity with any of SEQ ID NOs: 204, 208-213, 215, 216, 218, 222, or 223, or nucleic acid molecules encoding any MLH1 or variant of MLH1 (e.g., a dominant negative mutant of MLH1 as described herein), for inhibiting, blocking, or otherwise inactivating the wild type MLH1 function in the MMR pathway, and consequently, inhibiting, blocking, or otherwise inactivating the MMR pathway, e.g., during genome editing with a prime editor.

In some embodiments, inactivation of the MMR pathway involves an inhibitor that disrupts, blocks, interferes with, or otherwise inactivates the wild type function of the MLH1 protein. In some embodiments, inactivation of the MMR pathway involves a mutant of the MLH1 protein, for example, contacting a target cell with a MLH1 mutant protein or expressing in a target cell an MLH1 mutant nucleic acid that encodes an MLH1 mutant protein. In some embodiments, the MLH1 mutant protein interferes with, and thereby inactivates, the function of a wild type MLH1 protein in the MMR pathway. In some embodiments, the MLH1 mutant is a dominant negative mutant. In some embodiments, the MLH mutant protein is capable of binding to an MLH1-interacting protein, for example, MutS.

Without being bound by theory, MLH1 dominant negative mutants function by saturating binding of MutS, thereby blocking MutS-wild type MLH1 binding and interfering with the function of the wild type MLH1 protein in the MMR pathway.

In various embodiments, the dominant negative MLH1 can include, for example, MLH1 E34A, which is based on SEQ ID NO: 222 and having the following amino acid sequence (underline and bolded to show the E34A mutation):

(SEQ ID NO: 222)
MSFVAGVIRRLDETVVNRIAAGEVIQRPANAIKAMIENCLDAKSTSIQVIVKEGGLKLIQIQDNGTGIRKEDL
DIVCERFTTSKLQSFEDLASISTYGFRGEALASISHVAHVTITTKTADGKCAYRASYSDGKLKAPPKPCAGN
QGTQITVEDLFYNIATRRKALKNPSEEYGKILEVVGRYSVHNAGISFSVKKQGETVADVRTLPNASTVDNIR
SIFGNAVSRELIEIGCEDKTLAFKMNGYISNANYSVKKCIFLLFINHRLVESTSLRKAIETVYAAYLPKNTHPF
LYLSLEISPQNVDVNVHPTKHEVHFLHEESILERVQQHIESKLLGSNSSRMYFTQTLLPGLAGPSGEMVKST
TSLTSSSTSGSSDKVYAHQMVRTDSREQKLDAFLQPLSKPLSSQPQAIVTEDKTDISSGRARQQDEEMLELP
APAEVAAKNQSLEGDTTKGTSEMSEKRGPTSSNPRKRHREDSDVEMVEDDSRKEMTAACTPRRRIINLTSV
LSLQEEINEQGHEVLREMLHNHSFVGCVNPQWALAQHQTKLYLLNTTKLSEELFYQILIYDFANFGVLRLS
EPAPLFDLAMLALDSPESGWTEEDGPKEGLAEYIVEFLKKKAEMLADYFSLEIDEEGNLIGLPLLIDNYVPPL
EGLPIFILRLATEVNWDEEKECFESLSKECAMFYSIRKQYISEESTLSGQQSEVPGSIPNSWKWTVEHIVYKA
LRSHILPPKHFTEDGNILQLANLPDLYKVFERC,

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 222.

In various other embodiments, the dominant negative MLH1 can include, for example, MLH1 Δ756, which is based on SEQ ID NO: 208 and having the following amino acid sequence (underline and bolded to show the Δ756 mutation at the C terminus of the sequence):

(SEQ ID NO: 208)
MSFVAGVIRRLDETVVNRIAAGEVIQRPANAIKEMIENCLDAKSTSIQVIVKEGGLKLIQIQDNGTGIRKEDL
DIVCERFTTSKLQSFEDLASISTYGFRGEALASISHVAHVTITTKTADGKCAYRASYSDGKLKAPPKPCAGN
QGTQITVEDLFYNIATRRKALKNPSEEYGKILEVVGRYSVHNAGISFSVKKQGETVADVRTLPNASTVDNIR
SIFGNAVSRELIEIGCEDKTLAFKMNGYISNANYSVKKCIFLLFINHRLVESTSLRKAIETVYAAYLPKNTHPF
LYLSLEISPQNVDVNVHPTKHEVHFLHEESILERVQQHIESKLLGSNSSRMYFTQTLLPGLAGPSGEMVKST
TSLTSSSTSGSSDKVYAHQMVRTDSREQKLDAFLQPLSKPLSSQPQAIVTEDKTDISSGRARQQDEEMLELP
APAEVAAKNQSLEGDTTKGTSEMSEKRGPTSSNPRKRHREDSDVEMVEDDSRKEMTAACTPRRRIINLTSV
LSLQEEINEQGHEVLREMLHNHSFVGCVNPQWALAQHQTKLYLLNTTKLSEELFYQILIYDFANFGVLRLS
EPAPLFDLAMLALDSPESGWTEEDGPKEGLAEYIVEFLKKKAEMLADYFSLEIDEEGNLIGLPLLIDNYVPPL
EGLPIFILRLATEVNWDEEKECFESLSKECAMFYSIRKQYISEESTLSGQQSEVPGSIPNSWKWTVEHIVYKA
LRSHILPPKHFTEDGNILQLANLPDLYKVFER[−],

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 208 (wherein the [−] indicates deleted amino acid residue(s) relative to the parent or wildtype sequence).

In still other embodiments, the dominant negative MLH1 can include, for example, MLH1 Δ754-Δ756, which is based on SEQ ID NO: 209 and having the following amino acid sequence (underline and bolded to show the Δ754-Δ756 mutation at the C terminus of the sequence):

(SEQ ID NO: 209)
MSFVAGVIRRLDETVVNRIAAGEVIQRPANAIKEMIENCLDAKSTSIQVIVKEGGLKLIQIQDNGTGIRKEDL
DIVCERFTTSKLQSFEDLASISTYGFRGEALASISHVAHVTITTKTADGKCAYRASYSDGKLKAPPKPCAGN
QGTQITVEDLFYNIATRRKALKNPSEEYGKILEVVGRYSVHNAGISFSVKKQGETVADVRTLPNASTVDNIR
SIFGNAVSRELIEIGCEDKTLAFKMNGYISNANYSVKKCIFLLFINHRLVESTSLRKAIETVYAAYLPKNTHPF
LYLSLEISPQNVDVNVHPTKHEVHFLHEESILERVQQHIESKLLGSNSSRMYFTQTLLPGLAGPSGEMVKST
TSLTSSSTSGSSDKVYAHQMVRTDSREQKLDAFLQPLSKPLSSQPQAIVTEDKTDISSGRARQQDEEMLELP
APAEVAAKNQSLEGDTTKGTSEMSEKRGPTSSNPRKRHREDSDVEMVEDDSRKEMTAACTPRRRIINLTSV
LSLQEEINEQGHEVLREMLHNHSFVGCVNPQWALAQHQTKLYLLNTTKLSEELFYQILIYDFANFGVLRLS
EPAPLFDLAMLALDSPESGWTEEDGPKEGLAEYIVEFLKKKAEMLADYFSLEIDEEGNLIGLPLLIDNYVPPL
EGLPIFILRLATEVNWDEEKECFESLSKECAMFYSIRKQYISEESTLSGQQSEVPGSIPNSWKWTVEHIVYKA
LRSHILPPKHFTEDGNILQLANLPDLYKVF[---],

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 209 (wherein the [- - - ] indicates deleted amino acid residue(s) relative to the parent or wildtype sequence).

In yet other embodiments, the dominant negative MLH1 can include, for example, MLH1 E34A Δ754-Δ756, which is based on SEQ ID NO: 210 and having the following amino acid sequence (underline and bolded to show the E34A and Δ754-Δ756 mutations):

(SEQ ID NO: 210)
MSFVAGVIRRLDETVVNRIAAGEVIQRPANAIKAMIENCLDAKSTSIQVIVKEGGLKLIQIQDNGTGIRKEDL
DIVCERFTTSKLQSFEDLASISTYGFRGEALASISHVAHVTITTKTADGKCAYRASYSDGKLKAPPKPCAGN
QGTQITVEDLFYNIATRRKALKNPSEEYGKILEVVGRYSVHNAGISFSVKKQGETVADVRTLPNASTVDNIR
SIFGNAVSRELIEIGCEDKTLAFKMNGYISNANYSVKKCIFLLFINHRLVESTSLRKAIETVYAAYLPKNTHPF
LYLSLEISPQNVDVNVHPTKHEVHFLHEESILERVQQHIESKLLGSNSSRMYFTQTLLPGLAGPSGEMVKST
TSLTSSSTSGSSDKVYAHQMVRTDSREQKLDAFLQPLSKPLSSQPQAIVTEDKTDISSGRARQQDEEMLELP
APAEVAAKNQSLEGDTTKGTSEMSEKRGPTSSNPRKRHREDSDVEMVEDDSRKEMTAACTPRRRIINLTSV
LSLQEEINEQGHEVLREMLHNHSFVGCVNPQWALAQHQTKLYLLNTTKLSEELFYQILIYDFANFGVLRLS
EPAPLFDLAMLALDSPESGWTEEDGPKEGLAEYIVEFLKKKAEMLADYFSLEIDEEGNLIGLPLLIDNYVPPL
EGLPIFILRLATEVNWDEEKECFESLSKECAMFYSIRKQYISEESTLSGQQSEVPGSIPNSWKWTVEHIVYKA
LRSHILPPKHFTEDGNILQLANLPDLYKVF[---],

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 210.

In certain embodiments, the dominant negative MLH1 can include, for example, MLH1 1-335, which is based on SEQ ID NO: 211 and having the following amino acid sequence (contains amino acids 1-335 of SEQ NO: 204):

(SEQ ID NO: 211)
MSFVAGVIRRLDETVVNRIAAGEVIQRPANAIKEMIENCLDAKSTSIQVIVKEGGLKLIQIQDNGTGIRKEDL
DIVCERFTTSKLQSFEDLASISTYGFRGEALASISHVAHVTITTKTADGKCAYRASYSDGKLKAPPKPCAGN
QGTQITVEDLFYNIATRRKALKNPSEEYGKILEVVGRYSVHNAGISESVKKQGETVADVRTLPNASTVDNIR
SIFGNAVSRELIEIGCEDKTLAFKMNGYISNANYSVKKCIFLLFINHRLVESTSLRKAIETVYAAYLPKNTHPF
LYLSLEISPQNVDVNVHPTKHEVHFLHEESILERVQQHIESKLL,

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 211.

In other embodiments, the dominant negative MLH1 can include, for example, MLH1 1-335 E34A, which is based on SEQ ID NO: 212 and having the following amino acid sequence (contains amino acids 1-335 of SEQ NO: 204 and a E34A mutation relative to SEQ ID NO: 204):

(SEQ ID NO: 212)
MSFVAGVIRRLDETVVNRIAAGEVIQRPANAIKAMIENCLDAKSTSIQVI
VKEGGLKLIQIQDNGTGIRKEDLDIVCERFTTSKLQSFEDLASISTYGFR
GEALASISHVAHVTITTKTADGKCAYRASYSDGKLKAPPKPCAGNQGTQI
TVEDLFYNIATRRKALKNPSEEYGKILEVVGRYSVHNAGISFSVKKQGET
VADVRTLPNASTVDNIRSIFGNAVSRELIEIGCEDKTLAFKMNGYISNAN
YSVKKCIFLLFINHRLVESTSLRKAIETVYAAYLPKNTHPFLYLSLEISP
QNVDVNVHPTKHEVHFLHEESILERVQQHIESKLL,

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 212.

In still other embodiments, the dominant negative MLH1 can include, for example, MLH1 1-335 NLS^{SV40} (or referred to as MLH1dn^{NTD}, which is based on SEQ ID NO: 204 and having the following amino acid sequence (contains amino acids 1-335 of SEQ NO: 204 and an NLS sequence of SV40):

(SEQ ID NO: 213)
MSFVAGVIRRLDETVVNRIAAGEVIQRPANAIKEMIENCLDAKSTSIQVI
VKEGGLKLIQIQDNGTGIRKEDLDIVCERFTTSKLQSFEDLASISTYGFR
GEALASISHVAHVTITTKTADGKCAYRASYSDGKLKAPPKPCAGNQGTQI
TVEDLFYNIATRRKALKNPSEEYGKILEVVGRYSVHNAGISFSVKKQGET
VADVRTLPNASTVDNIRSIFGNAVSRELIEIGCEDKTLAFKMNGYISNAN
YSVKKCIFLLFINHRLVESTSLRKAIETVYAAYLPKNTHPFLYLSLEISP
QNVDVNVHPTKHEVHFLHEESILERVQQHIESKLLPKKKRKV,

with the underlined and bolded portion referring to the NLS of SV40), or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 213.

In still other embodiments, the dominant negative MLH1 can include, for example, MLH1 1-335 NLS^{alternate} (which is based on SEQ ID NO: 204 and having the following amino acid sequence (contains amino acids 1-335 of SEQ NO: 204 and an alternate NLS sequence)):

(SEQ ID NO: 214)
MSFVAGVIRRLDETVVNRIAAGEVIQRPANAIKEMIENCLDAKSTSIQVI
VKEGGLKLIQIQDNGTGIRKEDLDIVCERFTTSKLQSFEDLASISTYGFR
GEALASISHVAHVTITTKTADGKCAYRASYSDGKLKAPPKPCAGNQGTQI
TVEDLFYNIATRRKALKNPSEEYGKILEVVGRYSVHNAGISESVKKQGET
VADVRTLPNASTVDNIRSIFGNAVSRELIEIGCEDKTLAFKMNGYISNAN
YSVKKCIFLLFINHRLVESTSLRKAIETVYAAYLPKNTHPFLYLSLEISP
QNVDVNVHPTKHEVHFLHEESILERVQQHIESKLL-[alternate NLS
sequence]-[alternate NLS sequence],

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 214. The alternate NLS sequence can be any suitable NLS sequence, including but not limited to:

| Description | Sequence | SEQ ID NO: |
|---|---|---|
| NLS | MKRTADGSEFESPKKKRKV | SEQ ID NO: 101 |
| NLS | MDSLLMNRRKFLYQFKNVRWAKGRRETYLC | SEQ ID NO: 1 |
| NLS of nucleoplasmin | AVKRPAATKKAGQAKKKKLD | SEQ ID NO: 133 |
| NLS of EGL-13 | MSRRRKANPTKLSENAKKLAKEVEN | SEQ ID NO: 134 |
| NLS of c-MYC | PAAKRVKLD | SEQ ID NO: 135 |
| NLS of TUS-protein | KLKIKRPVK | SEQ ID NO: 136 |
| NLS of polyoma large T-Ag | VSRKRPRP | SEQ ID NO: 137 |
| NLS of Hepatitis D virus antigen | EGAPPAKRAR | SEQ ID NO: 138 |
| NLS of murine p53 | PPQPKKKPLDGE | SEQ ID NO: 139 |
| NLS of PE1 and PE2 | SGGSKRTADGSEFEPKKKRKV | SEQ ID NO: 103 |

In some embodiments, an NLS sequence is appended to the N-terminus of a protein and begins with a methionine (“M”). In other embodiments, an NLS sequence may be appended at the C-terminus of a protein, or between multiple domains of a fusion protein, and does not begin with a methionine (i.e., the M in, for example, SEQ ID NOs: 101, 1, and 134 is not included in the NLS when it is appended at the C-terminus or between two domains in a fusion protein).

In still other embodiments, the dominant negative MLH1 can include, for example, MLH1 501-756, which corresponds to a C-terminal fragment of SEQ ID NO: 204 that corresponds to amino acids 501-756 of SEQ ID NO: 204:

(SEQ ID NO: 215)
INLTSVLSLQEEINEQGHEVLREMLHNHSFVGCVNPQWALAQHQTKLYLL
NTTKLSEELFYQILIYDFANFGVLRLSEPAPLFDLAMLALDSPESGWTEE
DGPKEGLAEYIVEFLKKKAEMLADYFSLEIDEEGNLIGLPLLIDNYVPPL
EGLPIFILRLATEVNWDEEKECFESLSKECAMFYSIRKQYISEESTLSGQ
QSEVPGSIPNSWKWTVEHIVYKALRSHILPPKHFTEDGNILQLANLPDLY
KVFERC,

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 215.

In still other embodiments, the dominant negative MLH1 can include, for example, MLH1 501-753, which corresponds to a C-terminal fragment of SEQ ID NO: 204 that corresponds to amino acids 501-753 of SEQ ID NO: 204:

(SEQ ID NO: 216)
INLTSVLSLQEEINEQGHEVLREMLHNHSFVGCVNPQWALAQHQTKLYLL
NTTKLSEELFYQILIYDFANFGVLRLSEPAPLFDLAMLALDSPESGWTEE
DGPKEGLAEYIVEFLKKKAEMLADYFSLEIDEEGNLIGLPLLIDNYVPPL
EGLPIFILRLATEVNWDEEKECFESLSKECAMFYSIRKQYISEESTLSGQ
QSEVPGSIPNSWKWTVEHIVYKALRSHILPPKHFTEDGNILQLANLPDLY
KVF[---],

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 216.

In still other embodiments, the dominant negative MLH1 can include, for example, MLH1 461-756, which is a C-terminal fragment of SEQ ID NO: 204 that corresponds to amino acids 461-756 of SEQ ID NO: 204:

(SEQ ID NO: 217)
KRGPTSSNPRKRHREDSDVEMVEDDSRKEMTAACTPRRRIINLTSVLSLQ
EEINEQGHEVLREMLHNHSFVGCVNPQWALAQHQTKLYLLNTTKLSEELF
YQILIYDFANFGVLRLSEPAPLFDLAMLALDSPESGWTEEDGPKEGLAEY
IVEFLKKKAEMLADYFSLEIDEEGNLIGLPLLIDNYVPPLEGLPIFILRL
ATEVNWDEEKECFESLSKECAMFYSIRKQYISEESTLSGQQSEVPGSIPN
SWKWTVEHIVYKALRSHILPPKHFTEDGNILQLANLPDLYKVFERC,

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 217.

In various embodiments, the dominant negative MLH1 can include, for example, MLH1 461-753, which is a C-terminal fragment of SEQ ID NO: 204 that corresponds to amino acids 461-753 of SEQ ID NO: 204:

(SEQ ID NO: 218)
KRGPTSSNPRKRHREDSDVEMVEDDSRKEMTAACTPRRRIINLTSVLSLQ
EEINEQGHEVLREMLHNHSFVGCVNPQWALAQHQTKLYLLNTTKLSEELF
YQILIYDFANFGVLRLSEPAPLFDLAMLALDSPESGWTEEDGPKEGLAEY
IVEFLKKKAEMLADYFSLEIDEEGNLIGLPLLIDNYVPPLEGLPIFILRL
ATEVNWDEEKECFESLSKECAMFYSIRKQYISEESTLSGQQSEVPGSIPN
SWKWTVEHIVYKALRSHILPPKHFTEDGNILQLANLPDLYKVF[---],

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 218.

In various other embodiments, the dominant negative MLH1 can include, for example, MLH1 461-753, which is a C-terminal fragment of SEQ ID NO: 204 that corresponds to amino acids 461-753 of SEQ ID NO: 204, and which further comprises an N-terminal NLS, e.g., NLS^{SV40}:

(SEQ ID NO: 218)
[NLS]-KRGPTSSNPRKRHREDSDVEMVEDDSRKEMTAACTPRRRIINLT
SVLSLQEEINEQGHEVLREMLHNHSFVGCVNPQWALAQHQTKLYLLNTTK
LSEELFYQILIYDFANFGVLRLSEPAPLFDLAMLALDSPESGWTEEDGPK
EGLAEYIVEFLKKKAEMLADYFSLEIDEEGNLIGLPLLIDNYVPPLEGLP
IFILRLATEVNWDEEKECFESLSKECAMFYSIRKQYISEESTLSGQQSEV
PGSIPNSWKWTVEHIVYKALRSHILPPKHFTEDGNILQLANLPDLYKVF
[---],

or an amino acid sequence having at least 70%, at least 75%, at least 80%, at least 85%, at least 90%, at least 95%, at least 96%, at least 97%, at least 98%, at least 99%, or up to and including 100% sequence identity with SEQ ID NO: 218. The NLS sequence can be any suitable NLS sequence, including but not limited to SEQ ID NOs: 1, 101, 103, 133-139.
   napDNAbp

As used herein, the term “nucleic acid programmable DNA binding protein” or “napDNAbp,” of which Cas9 is an example, refer to proteins that use RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence.

Without being bound by theory, the binding mechanism of a napDNAbp—guide RNA complex, in general, includes the step of forming an R-loop whereby the napDNAbp induces the unwinding of a double-strand DNA target, thereby separating the strands in the region bound by the napDNAbp. The guide RNA spacer sequence then hybridizes to the “target strand.” This displaces a “non-target strand” that is complementary to the target strand, which forms the single strand region of the R-loop. In some embodiments, the napDNAbp includes one or more nuclease activities, which then cut the DNA, leaving various types of lesions. For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”). Exemplary sequences for these and other napDNAbp are provided herein.

### Nickase

The term “nickase.” as used herein, may refer to a Cas9 with one of the two nuclease domains inactivated. This enzyme is capable of cleaving only one strand of a target DNA. As used herein, a “nickase” may refer to a napDNAbp (e.g., a Cas protein) which is capable of cleaving only one of the two complementary strands of a double-stranded target DNA sequence, thereby generating a nick in that strand. In some embodiments, the nickase cleaves a non-target strand of a double stranded target DNA sequence. In some embodiments, the nickase comprises an amino acid sequence with one or more mutations in a catalytic domain of a canonical napDNAbp (e.g., a Cas protein), wherein the one or more mutations reduces or abolishes nuclease activity of the catalytic domain. In certain embodiments, the napDNAbp is a Cas9 nickase, a Cas12a nickase, or a Cas12b1 nickase. In some embodiments, the nickase is a Cas9 that comprises one or more mutations in a RuvC-like domain relative to a wild type Cas9 sequence or to an equivalent amino acid position in other Cas9 variants or Cas9 equivalents. In some embodiments, the nickase is a Cas9 that comprises one or more mutations in an HNH-like domain relative to a wild type Cas9 sequence or to an equivalent amino acid position in other Cas9 variants or Cas9 equivalents. In some embodiments, the nickase is a Cas9 that comprises an aspartate-to-alanine substitution (D10A) in the RuvC I catalytic domain of Cas9 relative to a canonical Cas9 sequence or to an equivalent amino acid position in other Cas9 variants or Cas9 equivalents. In some embodiments, the nickase is a Cas9 that comprises a H840A, N854A, and/or N863A mutation relative to a canonical Cas9 sequence, or to an equivalent amino acid position in other Cas9 variants or Cas9 equivalents. In some embodiments, the term “Cas9 nickase” refers to a Cas9 with one of the two nuclease domains inactivated. This enzyme is capable of cleaving only one strand of a target DNA. In some embodiments, the nickase is a Cas protein that is not a Cas9 nickase.

In some embodiments, the napDNAbp of the prime editing complex comprises an endonuclease having nucleic acid programmable DNA binding ability. In some embodiments, the napDNAbp comprises an active endonuclease capable of cleaving both strands of a double stranded target DNA. In some embodiments, the napDNAbp is a nuclease active endonuclease, e.g., a nuclease active Cas protein, that can cleave both strands of a double stranded target DNA by generating a nick on each strand. For example, a nuclease active Cas protein can generate a cleavage (a nick) on each strand of a double stranded target DNA. In some embodiments, the two nicks on both strands are staggered nicks, for example, generated by a napDNAbp comprising a Cas12a or Cas12b1. In some embodiments, the two nicks on both strands are at the same genomic position, for example, generated by a napDNAbp comprising a nuclease active Cas9. In some embodiments, the napDNAbp comprises an endonuclease that is a nickase. For example, in some embodiments, the napDNAbp comprises an endonuclease comprising one or more mutations that reduce nuclease activity of the endonuclease, rendering it a nickase. In some embodiments, the napDNAbp comprises an inactive endonuclease. For example, in some embodiments, the napDNAbp comprises an endonuclease comprising one or more mutations that abolish the nuclease activity. In various embodiments, the napDNAbp is a Cas9 protein or variant thereof. The napDNAbp can also be a nuclease active Cas9, a nuclease inactive Cas9 (dCas9), or a Cas9 nickase (nCas9). In a preferred embodiment, the napDNAbp is Cas9 nickase (nCas9) that nicks only a single strand. In certain embodiments, the napDNAbp is a Cas9 nickase, a Cas12a nickase, or a Cas12b1 nickase. In some embodiments, the napDNAbp can be selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute, and optionally has a nickase activity such that only one strand is cut. In some embodiments, the napDNAbp is selected from Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute, and optionally has a nickase activity such that one DNA strand is cut preferentially to the other DNA strand.

### Nick Site

The terms “cleavage site,” “nick site.” and “cut site” as used interchangeably herein in the context of prime editing, refer to a specific position in between two nucleotides or two base pairs in the double-stranded target DNA sequence. In some embodiments, the position of a nick site is determined relative to the position of a specific PAM sequence. In some embodiments, the nick site is the particular position where a nick will occur when the double stranded target DNA is contacted with a napDNAbp, e.g., a nickase such as a Cas nickase, that recognizes a specific PAM sequence. For each PEgRNA described herein, a nick site is characteristic of the particular napDNAbp to which the gRNA core of the PEgRNA associates with, and is characteristic of the particular PAM required for recognition and function of the napDNAbp. For example, for a PEgRNA that comprises a gRNA core that associates with a SpCas9, the nick site in the phosphodiester bond between bases three (“−3” position relative to the position 1 of the PAM sequence) and four (“−4” position relative to position 1 of the PAM sequence).

In some embodiments, a nick site is in a target strand of the double-stranded target DNA sequence. In some embodiments, a nick site is in a non-target strand of the double-stranded target DNA sequence. In some embodiments, the nick site is in a protospacer sequence. In some embodiments, the nick site is adjacent to a protospacer sequence. In some embodiments, a nick site is downstream of a region, e.g., on a non-target strand, that is complementary to a primer binding site of a PEgRNA. In some embodiments, a nick site is downstream of a region, e.g., on a non-target strand, that binds to a primer binding site of a PEgRNA. In some embodiments, a nick site is immediately downstream of a region, e.g., on a non-target strand, that is complementary to a primer binding site of a PEgRNA. In some embodiments, the nick site is upstream of a specific PAM sequence on the non-target strand of the double stranded target DNA, wherein the PAM sequence is specific for recognition by a napDNAbp that associates with the gRNA core of a PEgRNA. In some embodiments, the nick site is downstream of a specific PAM sequence on the non-target strand of the double stranded target DNA, wherein the PAM sequence is specific for recognition by a napDNAbp that associates with the gRNA core of a PEgRNA. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 nucleotides upstream of the PAM sequence, and the PAM sequence is recognized by a Cas9 nickase, wherein the Cas9 nickase comprises a nuclease active HNH domain and a nuclease inactive RuvC domain. In some embodiments, the nick site is 2 base pairs upstream of the PAM sequence, and the PAM sequence is recognized by a S. thermophilus Cas9 nickase.

### Nucleic Acid Molecule

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, O(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

### P53

As used herein, “p53” refers to tumor protein 53. Among other functions, p53 plays a role in DNA damage and repair, specifically in its role in regulation of the cell cycle, apoptosis, and genomic stability. P53 can activate DNA repair proteins when DNA has been damaged. P53 may also arrest cell growth by holding the cell cycle at the G1/S regulation point on DNA damage recognition, thereby providing DNA repair proteins more time to fix the DNA damage before allowing the cell to continue the cell cycle. Thus, in some embodiments of the methods described herein, p53 is inhibited (e.g., by the p53 inhibitor protein “i53.” or another p53 inhibitor) to increase the efficiency of prime editing.

### PEgRNA

As used herein, the terms “prime editing guide RNA” or “PEgRNA” or “extended guide RNA” refer to a specialized form of a guide RNA that has been modified to include one or more additional sequences for implementing the prime editing methods and compositions described herein. As described herein, the prime editing guide RNA comprise one or more “extended regions” of nucleic acid sequence. The extended regions may comprise, but are not limited to, single-stranded RNA or DNA. Further, the extended regions may occur at the 3′ end of a traditional guide RNA. In other arrangements, the extended regions may occur at the 5′ end of a traditional guide RNA. In still other arrangements, the extended region may occur at an intramolecular region of the traditional guide RNA, for example, in the gRNA core region which associates and/or binds to the napDNAbp. The extended region comprises a “DNA synthesis template” which encodes (by the polymerase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “spacer or linker” sequence, or other structural elements, such as, but not limited to aptamers, stem loops, hairpins, toe loops (e.g., a 3′ toeloop), or an RNA-protein recruitment domain (e.g., MS2 hairpin). As used herein the “primer binding site” comprises a sequence that hybridizes to a single-strand DNA sequence having a 3′end generated from the nicked DNA of the R-loop.

In certain embodiments, the PEgRNAs have a 5′ extension arm, a spacer, and a gRNA core. The 5′ extension further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In certain other embodiments, the PEgRNAs have a 5′ extension arm, a spacer, and a gRNA core. The 5′ extension further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In still other embodiments, the PEgRNAs have in the 5′ to 3′ direction a spacer (1), a gRNA core (2), and an extension arm (3). The extension arm (3) is at the 3′ end of the PEgRNA. The extension arm (3) further comprises in the 5′ to 3′ direction a “homology arm,” an “edit template.” and a “primer binding site.” In certain embodiments, a PEgRNA comprises from 5′ to 3′, a space, a DNA synthesis template, and a primer binding site. The extension arm (3) may also comprise an optional modifier region at the 3′ and 5′ ends, which may be the same sequences or different sequences. In addition, the 3′ end of the PEgRNA may comprise a transcriptional terminator sequence. These sequence elements of the PEgRNAs are further described and defined herein.

In still other embodiments, the PEgRNAs have in the 5′ to 3′ direction an extension arm (3), a spacer (1), and a gRNA core (2). The extension arm (3) is at the 5′ end of the PEgRNA. The extension arm (3) further comprises in the 3′ to 5′ direction a “homology arm,” an “edit template.” and a “primer binding site.” The extension arm (3) may also comprise an optional modifier region at the 3′ and 5′ ends, which may be the same sequences or different sequences. The PEgRNAs may also comprise a transcriptional terminator sequence at the 3′ end. These sequence elements of the PEgRNAs are further described and defined herein.

### PE1

As used herein, “PE1” refers to a prime editor system comprising a fusion protein comprising Cas9(H840A) and a wild type MMLV RT having the following structure: [NLS]-[Cas9(H840A)]-[linker]-[MMLV_RT(wt)]+a desired PEgRNA, wherein the PE fusion has the amino acid sequence of SEQ ID NO: 100, which is shown as follows;

(SEQ ID NO: 100)
MKRTADGSEFESPKKKRKVDKKYSIGLDIGTNSVGWAVITDEYKVPSKKF
KVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKY
PTIYHLRKKLVDSTDKADLRLIYLALAHMIKERGHFLIEGDLNPDNSDVD
KLFIQLVQTYNQLFEENPINASGVDAKAILSARLSKSRRLENLIAQLPGE
KKNGLFGNLIALSLGLTPNEKSNFDLAEDAKLQLSKDTYDDDLDNLLAQI
GDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEK
MDGTEELLVKLNREDLLRKQRTEDNGSIPHQIHLGELHAILRRQEDFYPF
LKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMK
RIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVA
QILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYG
GFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTID
RKRYTSTKEVLDATLIHQSITGLYETRIDLSQLGGDSGGSSGGSSGSETP
GTSESATPESSGGSSGGSSTLNIEDEYRLHETSKEPDVSLGSTWLSDFPQ
AWAETGGMGLAVRQAPLIIPLKATSTPVSIKQYPMSQEARLGIKPHIQRL
LDQGILVPCQSPWNTPLLPVKKPGTNDYRPVQDLREVNKRVEDIHPTVPN
PYNLLSGLPPSHQWYTVLDLKDAFFCLRLHPTSQPLFAFEWRDPEMGISG
QLTWTRLPQGFKNSPTLFDEALHRDLADFRIQHPDLILLQYVDDLLLAAT
SELDCQQGTRALLQTLGNLGYRASAKKAQICQKQVKYLGYLLKEGQRWLT
EARKETVMGQPTPKTPRQLREFLGTAGFCRLWIPGFAEMAAPLYPLTKTG
TLFNWGPDQQKAYQEIKQALLTAPALGLPDLTKPFELFVDEKQGYAKGVL
TQKLGPWRRPVAYLSKKLDPVAAGWPPCLRMVAAIAVLTKDAGKLTMGQP
LVILAPHAVEALVKQPPDRWLSNARMTHYQALLLDTDRVQFGPVVALNPA
TLLPLPEEGLQHNCLDILAEAHGTRPDLTDQPLPDADHTWYTDGSSLLQE
GQRKAGAAVTTETEVIWAKALPAGTSAQRAELIALTQALKMAEGKKLNVY
TDSRYAFATAHIHGEIYRRRGLLTSEGKEIKNKDEILALLKALFLPKRLS
IIHCPGHQKGHSAEARGNRMADQAARKAAITETPDTSTLLIENSSPSGGS
KRTADGSEFEPKKKRKV
KEY:
NUCLEAR LOCALIZATION SEQUENCE (NLS) TOP: (SEQ ID
NO: 101), BOTTOM: (SEQ ID NO: 103)
CAS9(H840A)(SEQ ID NO: 37)(SEQ ID NO: 37 corre-
sponds identically to SEQ ID NO: 2, except with an
H840A substitution)
33-AMINO ACID LINKER (SEQ ID NO: 102)
M-MLV reverse transcriptase (SEQ ID NO: 81).

Alternatively, PE1 may also refer to the prime editor fusion protein of SEQ ID NO: 100, i.e., without the pegRNA complexed thereto. PE1 may be complexed with a pegRNA during operation and/or use in prime editing.

### PE2

As used herein, “PE2” refers to a prime editing system comprising a fusion protein comprising Cas9(H840A) and a variant MMLV RT having the following structure: [NLS]-[Cas9(H840A)]-[linker]-[MMLV_RT(D200N)(T330P)(L603W)(T306K)(W313F)]+a desired PEgRNA, wherein the PE fusion has the amino acid sequence of SEQ ID NO: 107, which is shown as follows:

(SEQ ID NO: 107)
MKRTADGSEFESPKKKRKVDKKYSIGLDIGTNSVGWAVITDEYKVPSKKF
KVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKY
PTIYHLRKKLVDSTDKADLRLIYLALAHMIKERGHFLIEGDLNPDNSDVD
KLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMK
RIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVA
QILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYG
GFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKY
VNFLYLASHYEKLKGSPEDNEQKQLFVEQHKHYLDEHIEQISEFSKRVIL
ADANLDKVLSAYNKHRDKPIREQAENIIHLFTLTNLGAPAAFKYFDTTID
RKRYTSTKEVLDATLIHQSITGLYETRIDLSQLGGDSGGSSGGSSGSETP
GTSESATPESSGGSSGGSSTLNIEDEYRLHETSKEPDVSLGSTWLSDFPQ
AWAETGGMGLAVRQAPLIIPLKATSTPVSIKQYPMSQEARLGIKPHIQRL
LDQGILVPCQSPWNTPLLPVKKPGTNDYRPVQDLREVNKRVEDIHPTVPN
PYNLLSGLPPSHQWYTVLDLKDAFFCLRLHPTSQPLFAFEWRDPEMGISG
QLTWTRLPQGFKNSPTLFNEALHRDLADFRIQHPDLILLQYVDDLLLAAT
SELDCQQGTRALLQTLGNLGYRASAKKAQICQKQVKYLGYLLKEGQRWLT
EARKETVMGQPTPKTPRQLREFLGKAGFCRLFIPGFAEMAAPLYPLTKPG
TLFNWGPDQQKAYQEIKQALLTAPALGLPDLTKPFELFVDEKQGYAKGVL
TQKLGPWRRPVAYLSKKLDPVAAGWPPCLRMVAAIAVLTKDAGKLTMGQP
LVILAPHAVEALVKQPPDRWLSNARMTHYQALLLDTDRVQFGPVVALNPA
TLLPLPEEGLQHNCLDILAEAHGTRPDLTDQPLPDADHTWYTDGSSLLQE
GQRKAGAAVTTETEVIWAKALPAGTSAQRAELIALTQALKMAEGKKLNVY
TDSRYAFATAHIHGEIYRRRGWLTSEGKEIKNKDEILALLKALFLPKRLS
IIHCPGHQKGHSAEARGNRMADQAARKAAITETPDTSTLLIENSSPSGGS
KRTADGSEFEPKKKRKV
KEY:
NUCLEAR LOCALIZATION SEQUENCE (NLS) TOP: (SEQ ID
NO: 101), BOTTOM: (SEQ ID NO: 103)
CAS9(H840A)(SEQ ID NO: 37)
33-AMINO ACID LINKER (SEQ ID NO: 102)
M-MLV reverse transcriptase (SEQ ID NO: 98).

Alternatively, PE2 may also refer to the prime editor fusion protein of SEQ ID NO: 107, i.e., without the pegRNA complexed thereto. PE2 may be complexed with a pegRNA during operation and/or use in prime editing.

### PE3

As used herein, “PE3” refers to PE2 plus a second-strand nicking guide RNA that complexes with the PE2 and introduces a nick in the non-edited DNA strand in order to induce preferential replacement of the edited strand.

### PE3b

As used herein. “PE3b” refers to PE3 but wherein the second-strand nicking guide RNA is designed for temporal control such that the second strand nick is not introduced until after the installation of the desired edit. This is achieved by designing a gRNA with a spacer sequence that matches only the edited strand, but not the original allele. Using this strategy, referred to hereafter as PE3b, mismatches between the protospacer and the unedited allele should disfavor nicking by the sgRNA until after the editing event on the PAM strand takes place.

### PE4

As used herein, “PE4” refers to a system comprising PE2 plus an MLH1 dominant negative protein (i.e., wild-type MLH1 with amino acids 754-756 truncated as described further herein) expressed in trans. In some embodiments, PE4 refers to a fusion protein comprising PE2 and an MLH1 dominant negative protein joined via an optional linker.

### PE5

As used herein, “PE5” refers to a system comprising PE3 plus an MLH1 dominant negative protein (i.e., wild-type MLH1 with amino acids 754-756 deleted as described further herein, which may be referred to as “MLH1 Δ754-756” or “MLH1dn”) expressed in trans. In some embodiments, PE5 refers to a fusion protein comprising PE3 and an MLH1 dominant negative protein joined via an optional linker.

### PEmax

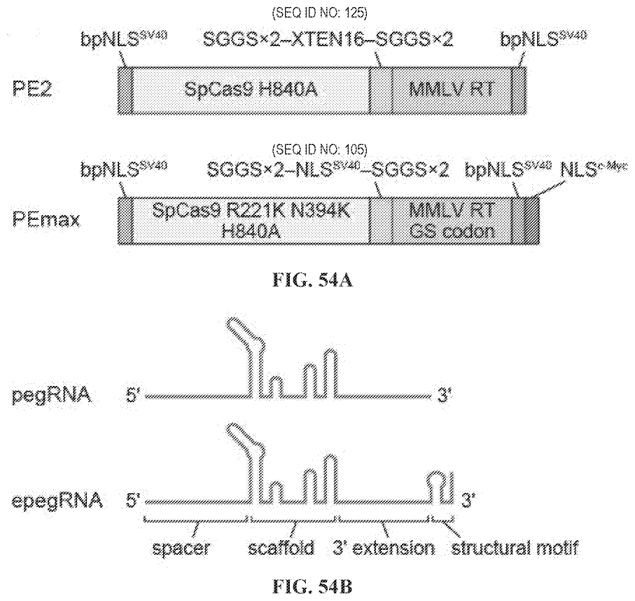

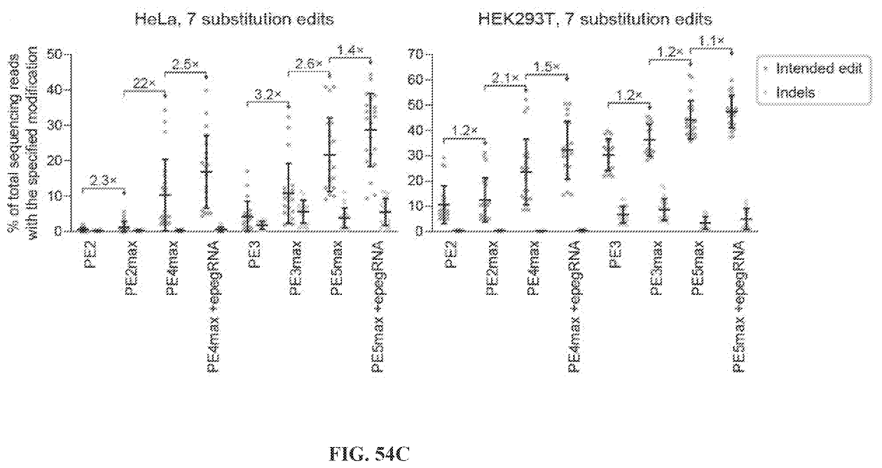

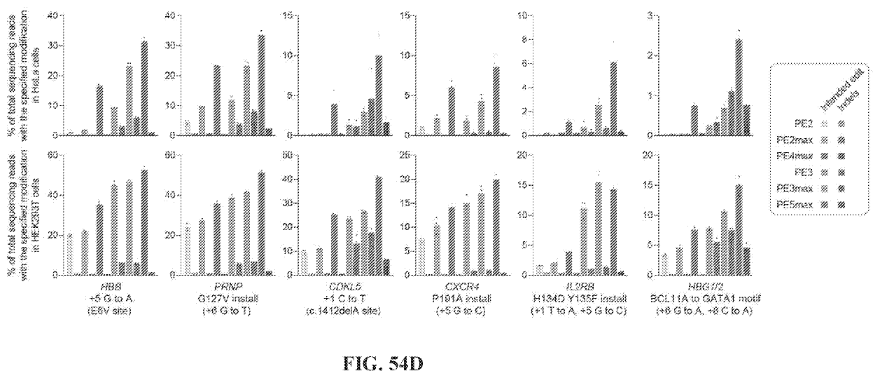

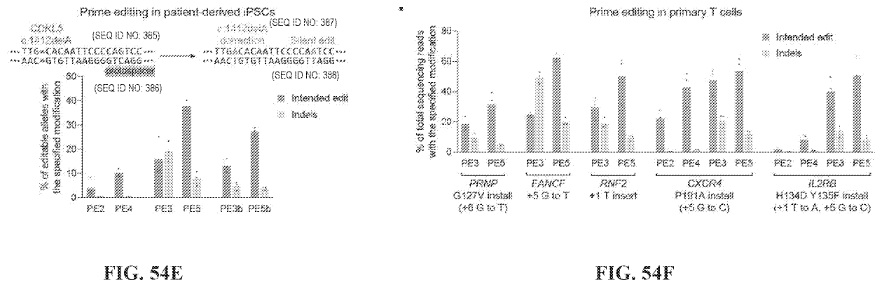

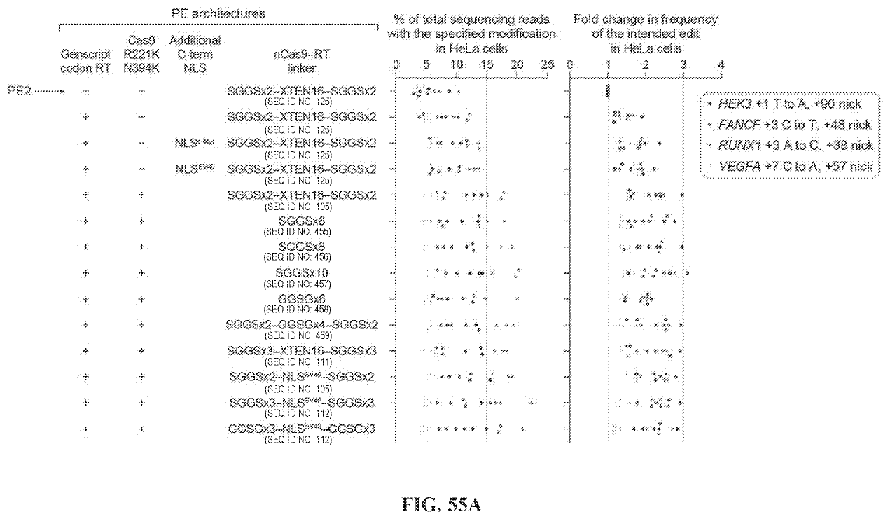

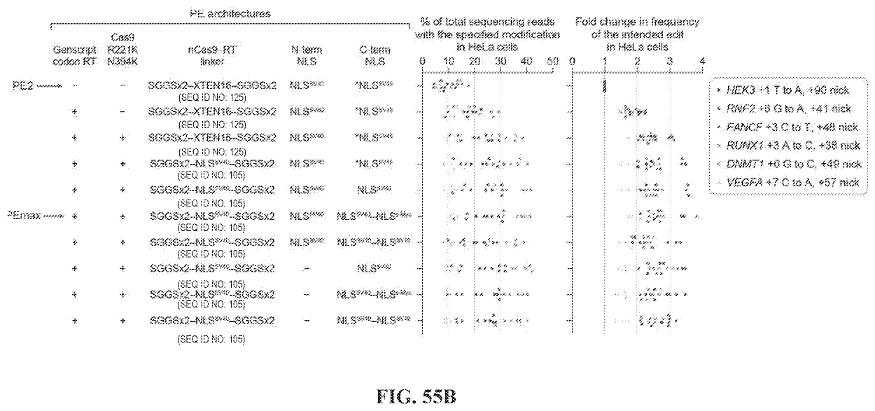

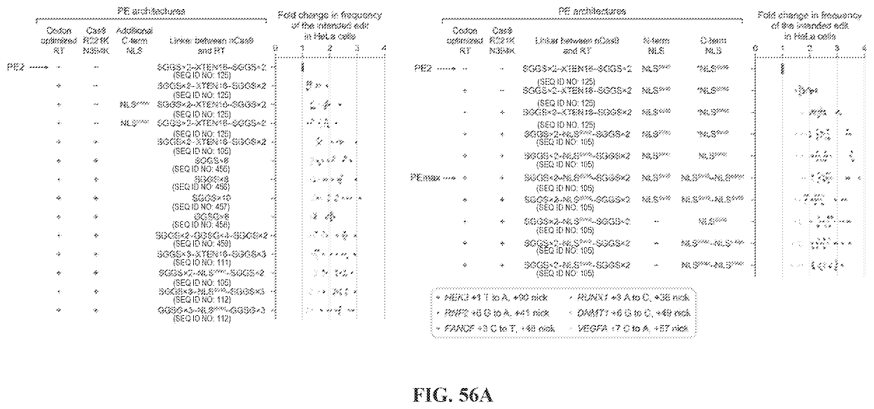

As used herein, “PEmax” (see FIG. 54B) refers to a PE complex comprising a fusion protein comprising Cas9(R221K N394K H840A) and a variant MMLV RT pentamutant (D200N T306K W313F T330P L603W) having the following structure: [bipartite NLS]-[Cas9(R221K)(N394K)(H840A)]-[linker]-[MMLV_RT(D200N)(T330P)(L603W)]-[bipartite NLS]-[NLS]+a desired PEgRNA, wherein the PE fusion has the amino acid sequence of SEQ ID NO: 99.

### PE4max

As used herein. “PE4max” refers to PE4 but wherein the PE2 component is substituted with PEmax.

### PE5max

As used herein, “PE5max” refers to PE5 but wherein the PE2 component of PE3 is substituted with PEmax.

### PE-short

As used herein, “PE-short” refers to a PE construct that is fused to a C-terminally truncated reverse transcriptase, and has the following amino acid sequence:

(SEQ ID NO: 117)
MKRTADGSEFESPKKKRKVDKKYSIGLDIGTNSVGWAVITDEYKVPSKKF
KVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISG
VEDRENASLGTYHDLLKIIKDKDFLDNEENEDILEDIVLTLTLFEDREMI
EERLKTYAHLEDDKVMKQLKRRRYTGWGRLSRKLINGIRDKQSGKTILDF
LKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMK
RIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNY
WRQLLNAKLITQRKEDNLTKAERGGLSELDKAGFIKRQLVETRQITKHVA
QILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYG
GFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKY
VNFLYLASHYEKLKGSPEDNEQKQLEVEQHKHYLDEIIEQISEFSKRVIL
ADANLDKVLSAYNKHRDKPIREQAENIIHLFTLTNLGAPAAFKYFDTTID
RKRYTSTKEVLDATLIHQSITGLYETRIDLSQLGGDSGGSSGGSSGSETP
GTSESATPESSGGSSGGSSTLNIEDEYRLHETSKEPDVSLGSTWLSDFPQ
AWAETGGMGLAVRQAPLIIPLKATSTPVSIKQYPMSQEARLGIKPHIQRL
LDQGILVPCQSPWNTPLLPVKKPGTNDYRPVQDLREVNKRVEDIHPTVPN
PYNLLSGLPPSHQWYTVLDLKDAFFCLRLHPTSQPLFAFEWRDPEMGISG
QLTWTRLPQGFKNSPTLFNEALHRDLADFRIQHPDLILLQYVDDLLLAAT
SELDCQQGTRALLQTLGNLGYRASAKKAQICQKQVKYLGYLLKEGQRWLT
EARKETVMGQPTPKTPRQLREFLGKAGFCRLFIPGFAEMAAPLYPLTKPG
TLFNWGPDQQKAYQEIKQALLTAPALGLPDLTKPFELFVDEKQGYAKGVL
TQKLGPWRRPVAYLSKKLDPVAAGWPPCLRMVAAIAVLTKDAGKLTMGQP
LVILAPHAVEALVKQPPDRWLSNARMTHYQALLLDTDRVQFGPVVALNPA
TLLPLPEEGLQHNCLDNSRLINSGGSKRTADGSEFEPKKKRKV
KEY:
NUCLEAR LOCALIZATION SEQUENCE (NLS) TOP: (SEQ ID
NO: 101), BOTTOM: (SEQ ID NO: 103)
CAS9(H840A)(SEQ ID NO: 37)
33-AMINO ACID LINKER 1 (SEQ ID NO: 102)
M-MLV TRUNCATED reverse transcriptase (SEQ ID NO:
80)

### Polymerase

As used herein, the term “polymerase” refers to an enzyme that synthesizes a nucleotide strand and that may be used in connection with the prime editor systems described herein. The polymerase can be a “template-dependent” polymerase (i.e., a polymerase that synthesizes a nucleotide strand based on the order of nucleotide bases of a template strand). The polymerase can also be a “template-independent” polymerase (i.e., a polymerase that synthesizes a nucleotide strand without the requirement of a template strand). A polymerase may also be further categorized as a “DNA polymerase” or an “RNA polymerase.” In various embodiments, the prime editor system comprises a DNA polymerase. In various embodiments, the DNA polymerase can be a “DNA-dependent DNA polymerase” (i.e., whereby the template molecule is a strand of DNA). In such cases, the DNA template molecule can be a PEgRNA, wherein the extension arm comprises a strand of DNA. In such cases, the PEgRNA may be referred to as a chimeric or hybrid PEgRNA which comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the PEgRNA is RNA, i.e., including an RNA extension. The term “polymerase” may also refer to an enzyme that catalyzes the polymerization of nucleotide (i.e., the polymerase activity). Generally, the enzyme will initiate synthesis at the 3′-end of a primer annealed to a polynucleotide template sequence (e.g., such as a primer sequence annealed to the primer binding site of a PEgRNA) and will proceed toward the 5′ end of the template strand. A “DNA polymerase” catalyzes the polymerization of deoxynucleotides. As used herein in reference to a DNA polymerase, the term DNA polymerase includes a “functional fragment thereof.” A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

### Prime Editing

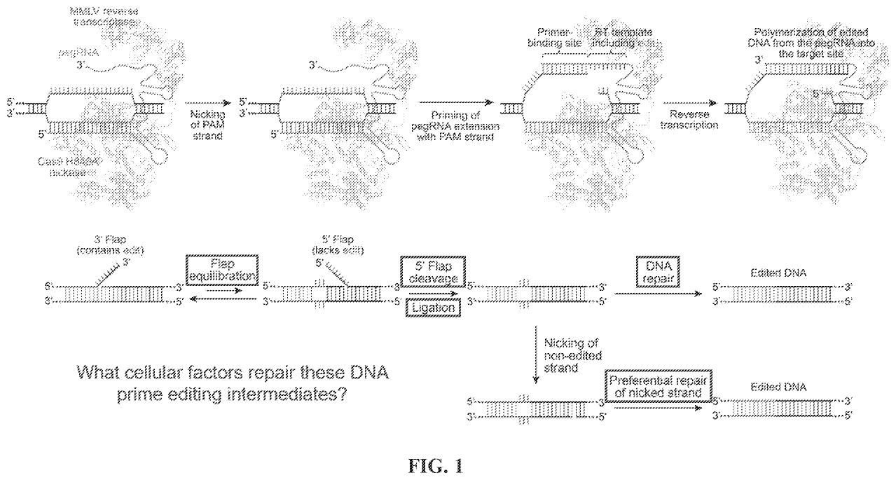

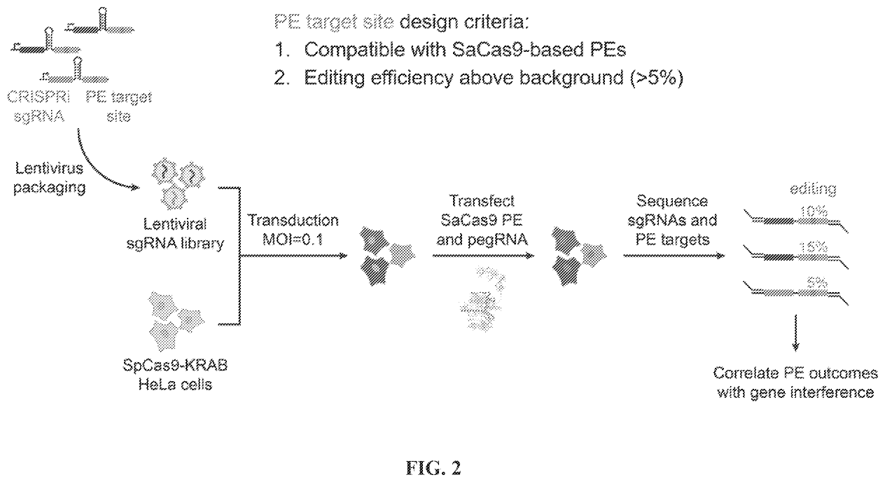

As used herein, the term “prime editing” refers to an approach for gene editing using napDNAbps, a polymerase (e.g., a reverse transcriptase), and specialized guide RNAs that include a DNA synthesis template for encoding desired new genetic information (or deleting genetic information) that is then incorporated into a target DNA sequence. Certain embodiments of prime editing are described in the embodiments of FIG. 1. Classical prime editing is described in the inventors' publication of Anzalone, A. V. et al. Search-and-replace genome editing without double-strand breaks or donor DNA. Nature 576, 149-157 (2019), which is incorporated herein by reference in its entirety.

Prime editing represents a platform for genome editing that is a versatile and precise genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same (or is homologous to) sequence as the endogenous strand (immediately downstream of the nick site) of the target site to be edited (with the exception that it includes the desired edit). Through DNA repair and/or replication machinery, the endogenous strand downstream of the nick site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit which is installed in place of the corresponding target site endogenous DNA strand. The prime editors of the present disclosure relate, in part, to the mechanism of target-primed reverse transcription (TPRT), which can be engineered for conducting precision CRISPR/Cas-based genome editing with high efficiency and genetic flexibility. TPRT is used by mobile DNA elements, such as mammalian non-LTR retrotransposons and bacterial Group II introns. The inventors have herein used Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, wherever the specification mentions a “reverse transcriptase,” the person having ordinary skill in the art should appreciate that any suitable DNA polymerase may be used in place of the reverse transcriptase. Thus, in one aspect, the prime editors may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. The specialized guide RNA also contains new genetic information in the form of an extension that encodes a replacement strand of DNA containing a desired genetic alteration which is used to replace a corresponding endogenous DNA strand at the target site. To transfer information from the PEgRNA to the target DNA, the mechanism of prime editing involves nicking the target site in one strand of the DNA to expose a 3′-hydroxyl group. The exposed Y-hydroxyl group can then be used to prime the DNA polymerization of the edit-encoding extension on PEgRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as, a reverse transcriptase). In the case of a DNA extension, the polymerase of the prime editor may be a DNA-dependent DNA polymerase. The newly synthesized strand (i.e., the replacement DNA strand containing the desired edit) that is formed by the herein disclosed prime editors would be homologous to the genomic target sequence (i.e., have the same sequence as) except for the inclusion of a desired nucleotide change (e.g., a single nucleotide change, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. In certain embodiments, the system can be combined with the use of an error-prone reverse transcriptase enzyme (e.g., provided as a fusion protein with the Cas9 domain, or provided in trans to the Cas9 domain). The error-prone reverse transcriptase enzyme can introduce alterations during synthesis of the single strand DNA flap. Thus, in certain embodiments, error-prone reverse transcriptase can be utilized to introduce nucleotide changes to the target DNA. Depending on the error-prone reverse transcriptase that is used with the system, the changes can be random or non-random. Resolution of the hybridized intermediate (comprising the single strand DNA flap synthesized by the reverse transcriptase hybridized to the endogenous DNA strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide change as a result of cellular DNA repair and/or replication processes. Because templated DNA synthesis offers single nucleotide precision for the modification of any nucleotide, including insertions and deletions, the scope of this approach is very broad and could foreseeably be used for myriad applications in basic science and therapeutics.

In various embodiments, prime editing operates by contacting a target DNA molecule (for which a change in the nucleotide sequence is desired to be introduced) with a nucleic acid programmable DNA binding protein (napDNAbp) complexed with a prime editing guide RNA (PEgRNA). In various embodiments, the prime editing guide RNA (PEgRNA) comprises an extension at the 3′ or 5′ end of the guide RNA, or at an intramolecular location in the guide RNA and encodes the desired nucleotide change (e.g., single nucleotide change, insertion, or deletion). In step (a), the napDNAbp/extended gRNA complex contacts the DNA molecule, and the extended gRNA guides the napDNAbp to bind to a target locus. In step (b), a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the protospacer of the extended gRNA) or the “non-target strand” (i.e., the strand forming the single-stranded portion of the R-loop and which is complementary to the target strand). In step (c), the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence” or “primer binding site” on the PEgRNA. In step (d), a reverse transcriptase (or other suitable DNA polymerase) is introduced which synthesizes a single strand of DNA from the 3′ end of the primed site towards the 5′ end of the prime editing guide RNA. The DNA polymerase (e.g., reverse transcriptase) can be fused to the napDNAbp or alternatively can be provided in trans to the napDNAbp. This forms a single-strand DNA flap comprising the desired nucleotide change (e.g., the single base change, insertion, or deletion, or a combination thereof) and which is otherwise homologous to the endogenous DNA at or adjacent to the nick site. In step (e), the napDNAbp and guide RNA are released. Steps (f) and (g) relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cells endogenous DNA repair and replication processes resolves the mismatched DNA to incorporate the nucleotide change(s) to form the desired altered product. The process can also be driven towards product formation with “second strand nicking.” This process may introduce at least one or more of the following genetic changes: transversions, transitions, deletions, and insertions.

The term “prime editor (PE) system” or “prime editor (PE)” or “PE system” or “PE editing system” refers the compositions involved in the method of genome editing using target-primed reverse transcription (TPRT) describe herein, including, but not limited to the napDNAbps, reverse transcriptases (or another DNA polymerase), fusion proteins (e.g., comprising napDNAbps and reverse transcriptases or comprising napDNAbps and DNA polymerases), prime editing guide RNAs, and complexes comprising fusion proteins and prime editing guide RNAs, as well as accessory elements, such as second strand nicking components (e.g., second strand sgRNAs) and 5′ endogenous DNA flap removal endonucleases (e.g., FEN1) for helping to drive the prime editing process towards the edited product formation.

Although in the embodiments described thus far the PEgRNA constitutes a single molecule comprising a guide RNA (which itself comprises a spacer sequence and a gRNA core or scaffold) and a 5′ or 3′ extension arm comprising the primer binding site and a DNA synthesis template, the PEgRNA may also take the form of two individual molecules comprised of a guide RNA and a trans prime editor RNA template (tPERT), which essentially houses the extension arm (including, in particular, the primer binding site and the DNA synthesis domain) and an RNA-protein recruitment domain (e.g., MS2 aptamer or hairpin) in the same molecule which becomes co-localized or recruited to a modified prime editor complex that comprises a tPERT recruiting protein (e.g., MS2cp protein, which binds to the MS2 aptamer).

### Prime Editor

The term “prime editor” refers to fusion constructs comprising a napDNAbp (e.g., Cas9 nickase) and a reverse transcriptase and is capable of carrying out prime editing on a target nucleotide sequence in the presence of a PEgRNA (or “extended guide RNA”). The term “prime editor” may refer to the fusion protein or to the fusion protein complexed with a PEgRNA, and/or further complexed with a second-strand nicking sgRNA. In some embodiments, the prime editor may also refer to the complex comprising a fusion protein (reverse transcriptase fused to a napDNAbp), a PEgRNA, and a regular guide RNA capable of directing the second-site nicking step of the non-edited strand as described herein. In certain embodiments, a prime editor (e.g., PE1, PE2, or PE3) may be provided as a system along with an inhibitor of the DNA mismatch repair pathway, such as a dominant negative MLH1 protein. In various embodiments, the inhibitor of the DNA mismatch repair pathway, such as a dominant negative MLH1 protein, may be provided in trans to the prime editor. In other embodiments, the inhibitor of the DNA mismatch repair pathway, such as a dominant negative MLH1 protein, may be complexed to the prime editor, e.g., coupled through a linker to the prime editor fusion protein.

### Primer Binding Site

The term “primer binding site” or “the PBS” refers to the portion of a PEgRNA as a component of the extension arm (for example, at the 3′ end of the extension arm). The term “primer binding site” refers to a single-stranded portion of the PEgRNA as a component of the extension arm that comprises a region of complementarity to a sequence on the non-target strand. In some embodiments, the primer binding site is complementary to a region upstream of a nick site in a non-target strand. In some embodiments, the primer binding site is complementary to a region immediately upstream of a nick site in the non-target strand. In some embodiments, the primer binding site is capable of binding to the primer sequence that is formed after nicking (e.g., by a nickase component of a prime editor, for example, a Cas9 nikcase) of the target sequence by the prime editor. When a prime editor nicks one strand of the target DNA sequence (e.g., by a Cas nickase component of the prime editor), a 3′-ended ssDNA flap is formed, which serves a primer sequence that anneals to the primer binding site on the PEgRNA to prime reverse transcription. In some embodiments, the PBS is complementary to, or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

### Protospacer

As used herein, the term “protospacer” refers to the sequence (˜20 bp) in DNA adjacent to the PAM (protospacer adjacent motif) sequence. The protospacer shares the same sequence as the spacer sequence of the guide RNA. The guide RNA anneals to the complement of the protospacer sequence on the target DNA (specifically, one strand thereof, i.e., the “target strand” versus the “non-target strand” of the target DNA sequence). In some embodiments, in order for a Cas nickase component of the prime editor to function it also requires a specific protospacer adjacent motif (PAM) which varies depending on the Cas protein component itself, e.g., the type of Cas protein. For example, the most commonly used Cas9 nuclease, derived from S. pyogenes, recognizes a PAM sequence of NGG that is found directly downstream of the target sequence in the genomic DNA, on the non-target strand. The skilled person will appreciate that the literature in the state of the art sometimes refers to the “protospacer” as the ˜20-nt target-specific guide sequence on the guide RNA itself, rather than referring to it as a “spacer.” Thus, in some cases, the term “protospacer” as used herein may be used interchangeably with the term “spacer.” The context of the description surrounding the appearance of either “protospacer” or “spacer” will help inform the reader as to whether the term is in reference to the gRNA or the DNA target.

### Protospacer Adjacent Motif (PAM)

As used herein, the term “protospacer adjacent sequence” or “PAM” refers to an approximately 2-6 base pair DNA sequence that is an important targeting component of a Cas9 nuclease. Typically, the PAM sequence is on either strand, and is downstream in the 5′ to 3′ direction of the Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes alternative PAM sequence.

For example, with reference to the canonical SpCas9 amino acid sequence is SEQ ID NO: 2, the PAM sequence can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These are examples and are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10(5): 891-899 (which is incorporated herein by reference).

### Reverse Transcriptase

The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA which can then be cloned into a vector for further manipulation. Avian myoblastosis virus (AMV) reverse transcriptase was the first widely used RNA-dependent DNA polymerase (Verma, Biochim. Biophys. Acta 473:1(1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNase H activity has been presented by Berger et al., Biochemistry 22:2365-2372 (1983). Another reverse transcriptase which is used extensively in molecular biology is reverse transcriptase originating from Moloney murine leukemia virus (M-MLV). See, e.g., Gerard, G. R., DNA 5:271-279 (1986) and Kotewicz, M. L., et al., Gene 35:249-258 (1985). M-MLV reverse transcriptase substantially lacking in RNase H activity has also been described. See, e.g., U.S. Pat. No. 5,244,797. The invention contemplates the use of any such reverse transcriptases, or variants or mutants thereof.

In addition, the invention contemplates the use of reverse transcriptases that are error-prone, i.e., that may be referred to as error-prone reverse transcriptases or reverse transcriptases that do not support high fidelity incorporation of nucleotides during polymerization. During synthesis of the single-strand DNA flap based on the RT template integrated with the guide RNA, the error-prone reverse transcriptase can introduce one or more nucleotides which are mismatched with the RT template sequence, thereby introducing changes to the nucleotide sequence through erroneous polymerization of the single-strand DNA flap. These errors introduced during synthesis of the single strand DNA flap then become integrated into the double strand molecule through hybridization to the corresponding endogenous target strand, removal of the endogenous displaced strand, ligation, and then through one more round of endogenous DNA repair and/or sequencing processes.

### Reverse Transcription

As used herein, the term “reverse transcription” indicates the capability of an enzyme to synthesize a DNA strand (that is, complementary DNA or cDNA) using RNA as a template. In some embodiments, the reverse transcription can be “error-prone reverse transcription,” which refers to the properties of certain reverse transcriptase enzymes which are error-prone in their DNA polymerization activity.

### Protein, Peptide, and Polypeptide

The terms “protein,” “peptide.” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a famesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual ⁽⁴th ed., Cold Spring Harbor Laboratory Press, Cold Spring Harbor. N.Y. (2012)), the entire contents of which are incorporated herein by reference.

### Silent Mutation

As used herein, the term “silent mutation” refers to a mutation in a nucleic acid molecule that does not have an effect on the phenotype of the nucleic acid molecule, or the protein it produces if it encodes a protein. Silent mutations can be present in coding regions of a nucleic acid (i.e., segments of a gene that encode for a protein), or they can be present in non-coding regions of a nucleic acid. A silent mutation in a nucleic acid sequence, e.g., in a target DNA sequence or in a DNA synthesis template sequence to be installed in the target sequence, may be a nucleotide alteration that does not result in expression or function of the amino acid sequence encoded by the nucleic acid sequence, or other functional features of the target nucleic acid sequence. When silent mutations are present in a coding region, they may be synonymous mutations. Synonymous mutations refer to substitutions of one base for another in a gene such that the corresponding amino acid residue of the protein produced by the gene is not modified. This is due to the redundancy of the genetic code, allowing for multiple different codons to encode for the same amino acid in a particular organism. When a silent mutation is in a non-coding region or a junction of a coding region and a non-coding region (e.g., an intron/exon junction), it may be in a region that does not impact any biological properties of the nucleic acid molecule (e.g., splicing, gene regulation, RNA lifetime, etc.). Silent mutations may be useful, for example, for increasing the length of an edit made to a target nucleotide sequence using prime editing to evade correction of the edit by the MMR pathway as described herein. In certain embodiments, the number of silent mutations installed may be one, or two, or three, or four, or five, or six, or seven, or eight, or nine, or ten, or more. In certain other embodiments involving at least two silent mutations, the silent mutations may be installed within one, or two, or three, or four, or five, or six, or seven, or eight, or nine, or ten, or 11, 12, 13, 14, 15, 16, 17, 18, 19, 20, 21, 22, 23, 24, or 25 nucleotides from the intended edit site.

### Spacer Sequence

As used herein, the term “spacer sequence” in connection with a guide RNA or a PEgRNA refers to the portion of the guide RNA or PEgRNA of about 20 nucleotides (e.g., 16, 17, 18, 19, 20, 21, 22, 23 or 24 nucleotides) which contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

### Target Site

The term “target site” refers to a sequence within a nucleic acid molecule to be edited by a prime editor (PE) disclosed herein. The target site may refer to the endogenous sequence within the nucleic acid molecule to be edited, e.g., endogenous genomic sequence of a target genome, which is identical to the sequence of the DNA synthesis template except for the one or more nucleotide edits to be installed present on the DNA synthesis template (and except that the DNA synthesis template contains Uracil instead of Thymine), or the corresponding endogenous sequence on the non-target strand that is complementary to the DNA synthesis template except for one or more mismatches at the position of the one or more nucleotide edits to be installed present on the DNA synthesis template. The target site may also further refer to the sequence within a nucleic acid molecule to which a complex of the prime editor (PE) and gRNA binds.

### Variant

As used herein the term “variant” should be taken to mean the exhibition of qualities that have a pattern that deviates from what occurs in nature. e.g., a variant Cas9 is a Cas9 comprising one or more changes in amino acid residues as compared to a wild type Cas9 amino acid sequence. The term “variant” encompasses homologous proteins having at least 75%, or at least 80%, or at least 85%, or at least 90%, or at least 95%, or at least 99% percent identity with a reference sequence and having the same or substantially the same functional activity or activities as the reference sequence. The term also encompasses mutants, truncations, or domains of a reference sequence, and which display the same or substantially the same functional activity or activities as the reference sequence.

### Vector

The term “vector,” as used herein, refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### DETAILED DESCRIPTION OF CERTAIN EMBODIMENTS

The present disclosure provides compositions and methods for prime editing with improved editing efficiency and/or reduced indel formation by inhibiting the DNA mismatch repair pathway while conducting prime editing of a target site. The inventors have surprisingly found that the editing efficiency of prime editing may be significantly increased (e.g., 2-fold increase, 3-fold increase, 4-fold increase, 5-fold increase, 6-fold increase, 7-fold increase, 8-fold increase, 9-fold increase, 10-fold increase, 1l-fold increase, 12-fold increase, 13-fold increase, 14-fold increase, 15-fold increase, 16-fold increase, 17-fold increase, 18-fold increase, 19-fold increase, 20-fold increase, 21-fold increase, 22-fold increase, 23-fold increase, 24-fold increase, 26-fold increase, 27-fold increase, 28-fold increase, 29-fold increase, 30-fold increase, 31-fold increase, 32-fold increase, 33-fold increase, 34-fold increase, 35-fold increase, 36-fold increase, 37-fold increase, 38-fold increase, 39-fold increase, 40-fold increase 0.41-fold increase, 42-fold increase, 43-fold increase, 44-fold increase, 45-fold increase, 46-fold increase, 47-fold increase, 48-fold increase, 49-fold increase, 50-fold increase, 51-fold increase, 52-fold increase, 53-fold increase, 54-fold increase, 55-fold increase, 56-fold increase, 57-fold increase, 58-fold increase, 59-fold increase, 60-fold increase, 61-fold increase, 62-fold increase, 63-fold increase, 64-fold increase, 65-fold increase, 66-fold increase, 67-fold increase, 68-fold increase, 69-fold increase, 70-fold increase, 71-fold increase, 72-fold increase, 73-fold increase, 74-fold increase, 75-fold increase, 76-fold increase, 77-fold increase, 78-fold increase, 79-fold increase, 80-fold increase, 81-fold increase, 82-fold increase, 83-fold increase, 84-fold increase, 85-fold increase, 86-fold increase, 87-fold increase, 88-fold increase, 89-fold increase, 90-fold increase, 91-fold increase, 92-fold increase, 93-fold increase, 94-fold increase, 95-fold increase, 96-fold increase, 97-fold increase, 98-fold increase, 99-fold increase, 100-fold increase or more) when one or more functions of the DNA mismatch repair (MMR) system are inhibited, blocked, or otherwise inactivated during prime editing. In addition, the inventors have surprisingly found that the frequency of indel formation resulting from prime editing may be significantly decreased (e.g., 2-fold decrease, 3-fold decrease, 4-fold decrease, 5-fold decrease, 6-fold decrease, 7-fold decrease, 8-fold decrease, 9-fold decrease, or 10-fold decrease or lower) when one or more functions of the DNA mismatch repair (MMR) system are inhibited, blocked, or otherwise inactivated during prime editing.

The disclosure relates to the surprising finding that the efficiency and/or specificity of prime editing is impacted by a cell's own DNA mismatch repair (MMR) DNA repair pathway. As described herein (e.g., in Example 1), the inventors developed a novel genetic screening method—referred to in one embodiment as “pooled CRISPRi screen for prime editing outcomes”—which led to the identification of various genetic determinates, including MMR, as affecting the efficiency and/or specificity of prime editing. Accordingly, in one aspect, the present disclosure provides novel prime editing systems comprising a means for inhibiting and/or evade the effects of MMR, thereby increasing the efficiency and/or specificity of prime editing. In one embodiment, the disclosure provides a prime editing system that comprises an MMR-inhibiting protein, such as, but not limited to, a dominant negative MMR protein, such as a dominant negative MLH1 protein (i.e., “MLH1dn”). In another embodiment, the prime editing system comprises the installation of one or more silent mutations nearby an intended edit, thereby allowing the intended edit from evading MMR recognition, even in the absence of an MMR-inhibiting protein, such as an MLH1dn. In another aspect, the disclosure provides a novel genetic screen for identifying genetic determinants, such as MMR, that impact the efficiency and/or specificity of prime editing. In still further aspects, the disclosure provides nucleic acid constructs encoding the improved prime editing systems described herein. The disclosure in other aspects also provides vectors (e.g., AAV or lentivirus vectors) comprising nucleic acids encoding the improved prime editing system described herein. In still other aspects, the disclosure provides cells comprising the improved prime editing systems described herein. The disclosure also provides in other aspects the components of the genetic screens, including nucleic acid and/or vector constructs, guide RNA, pegRNAs, cells (e.g., CRISPRi cells), and other reagents and/or materials for conducting the herein disclosed genetic screens. In still other aspects, the disclosure provides compositions and kits, e.g., pharmaceutical compositions, comprising the improved prime editing system described herein and which are capable of being administered to a cell, tissue, or organism by any suitable means, such as by gene therapy, mRNA delivery, virus-like particle delivery, or ribonucleoprotein (RNP) delivery. In yet another aspect, the present disclosure provides methods of using the improved prime editing system to install one or more edits in a target nucleic acid molecule, e.g., a genomic locus. In still another aspect, the present disclosure provides methods of treating a disease or disorder using the improved prime editing system to correct or otherwise repair one or more genetic changes (e.g., a single polymorphism) in a target nucleic acid molecule, e.g., a genomic locus comprising one or more disease-causing mutations.

In one embodiment, the MLH1 protein is inhibited, blocked, or otherwise inactivated. In other embodiments, other proteins of the MMR system are inhibited, blocked, or otherwise inactivated, including, but not limited to, PMS2 (or MutL alpha). PMS1 (or MutL beta), MLH3 (or MutL gamma), MutS alpha (MSH2-MSH6), MutS beta (MSH2-MSH3), MSH2, MSH6, PCNA, RFC, EXO1, POLδ, and PCNA. The inhibition may involve inhibiting the protein with an inhibitor (e.g., antibody or small molecule inhibitor or a dominant negative variant of the protein which disrupts, blocks, or otherwise inactivates the function of the protein, e.g., a dominant negative form of MLH1). The inhibition may also involve any other suitable means, such as by protein degradation (e.g., PROTAC-based degradation of MLH1), transcript-level inhibition (e.g., siRNA transcript degradation/gene silencing or microRNA-based inhibition of translation of the MLH1 transcript), or at the genetic level (i.e., installing a mutation in the MLH1 gene (or regulatory regions) which inactivates or reduces the expression of the MLH1 gene, or which installs a mutation which inactivates, blocks, or minimizes that activity of the encoded MLH1 product). In addition, the disclosure contemplates that the prime editor (e.g., delivered as a fusion protein comprising a napDNAbp and a polymerase, such as a Cas9 nickase fused to a reverse transcriptase) may be administered together with any inhibitor of the DNA mismatch repair pathway.

Accordingly, the present disclosure provides a method for editing a nucleic acid molecule by prime editing that involves contacting a nucleic acid molecule with a prime editor, a pegRNA, and an inhibitor of the DNA mismatch repair pathway, thereby installing one or more modifications to the nucleic acid molecule at a target site with increased editing efficiency and/or lower indel formation. The present disclosure further provides polynucleotides for editing a DNA target site by prime editing comprising a nucleic acid sequence encoding a napDNAbp, a polymerase, and an inhibitor of the DNA mismatch repair pathway, wherein the napDNAbp and polymerase is capable in the presence of a pegRNA of installing one or more modifications in the DNA target site with increased editing efficiency and/or lower indel formation. The disclosure further provides, vectors, cells, and kits comprising the compositions and polynucleotides of the disclosure, as well as methods of making such vectors, cells, and kits, as well as methods for delivery such compositions, polynucleotides, vectors, cells and kits to cells in vitro, ex vivo (e.g., during cell-based therapy which modify cells outside of the body), and in vivo.

### MMR Pathway

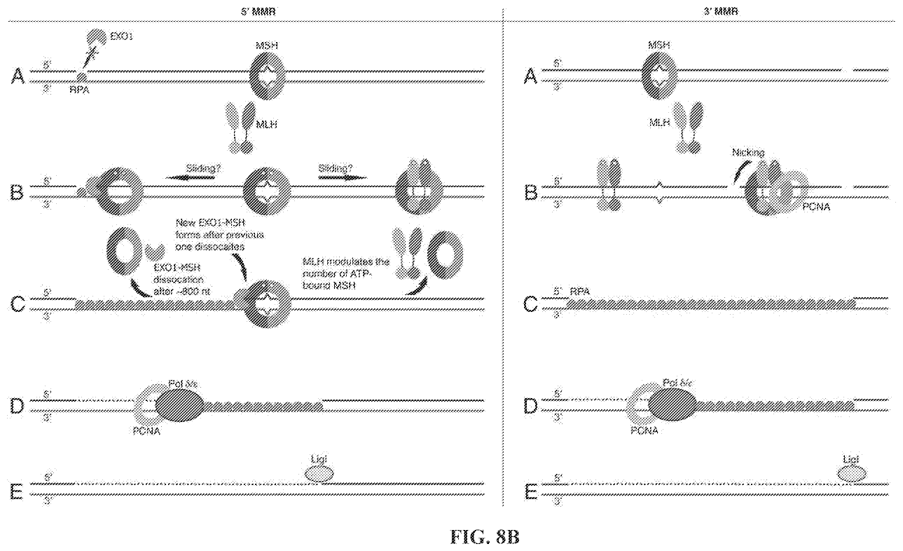

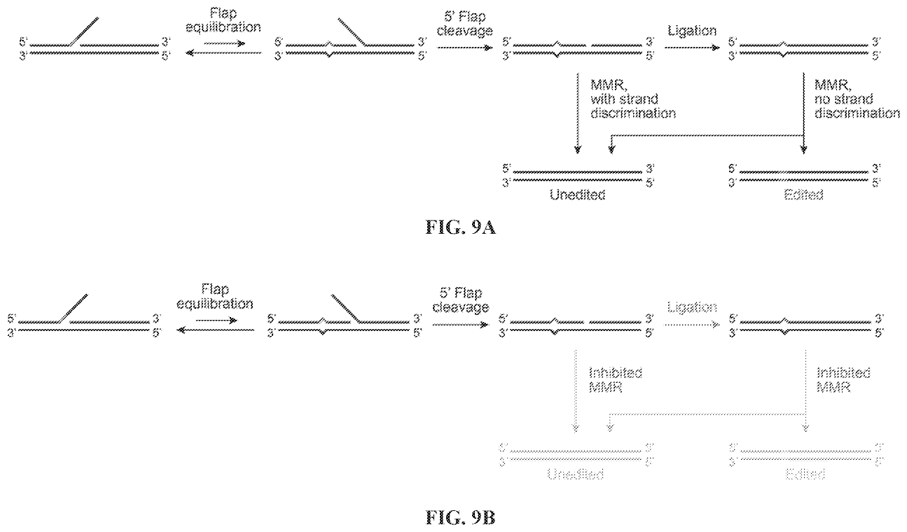

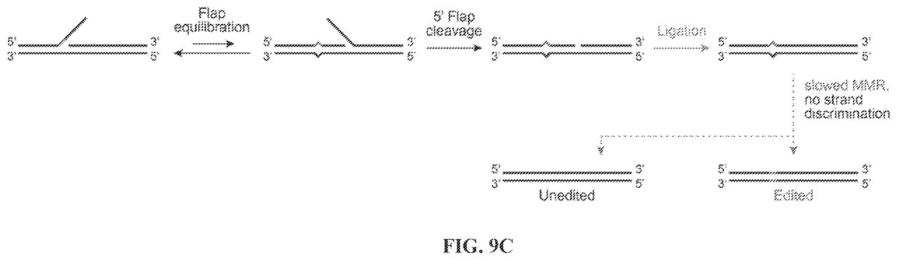

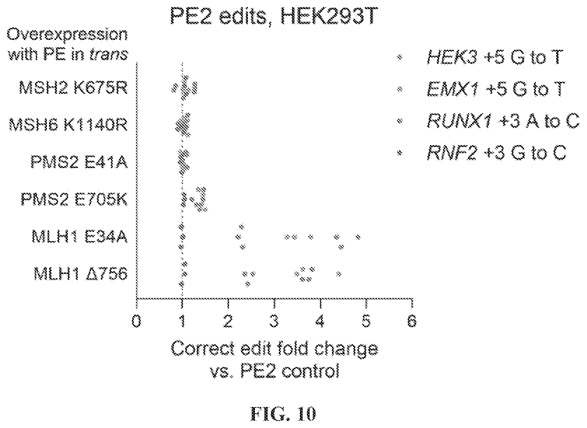

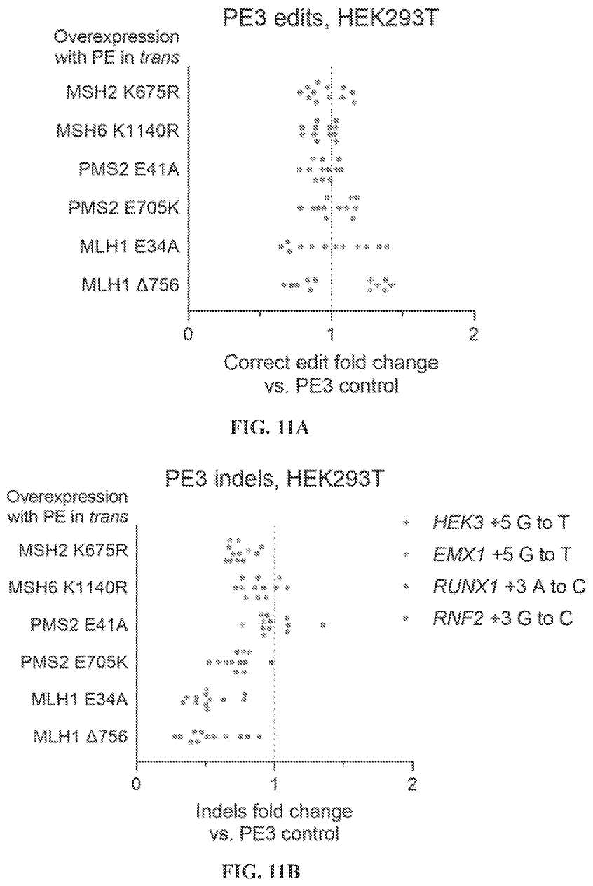

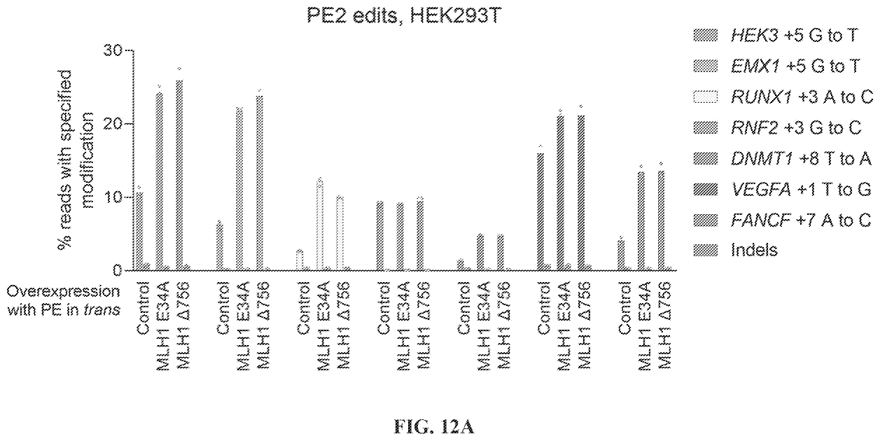

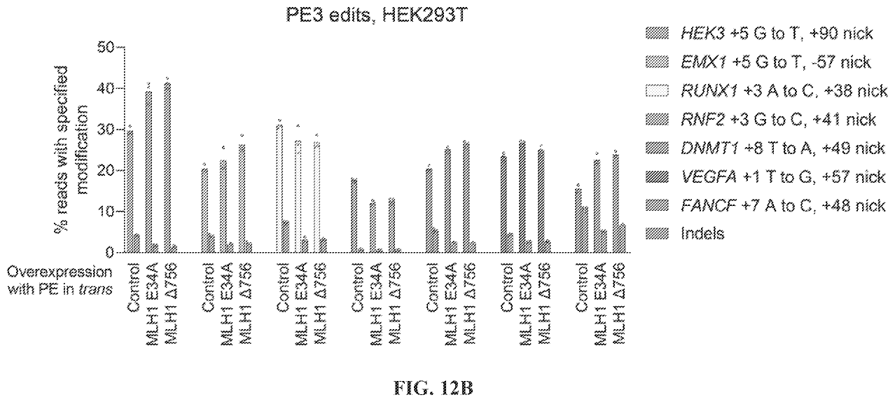

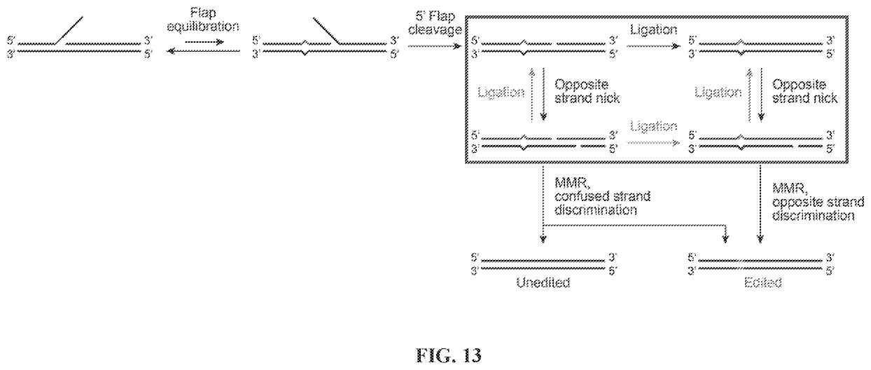

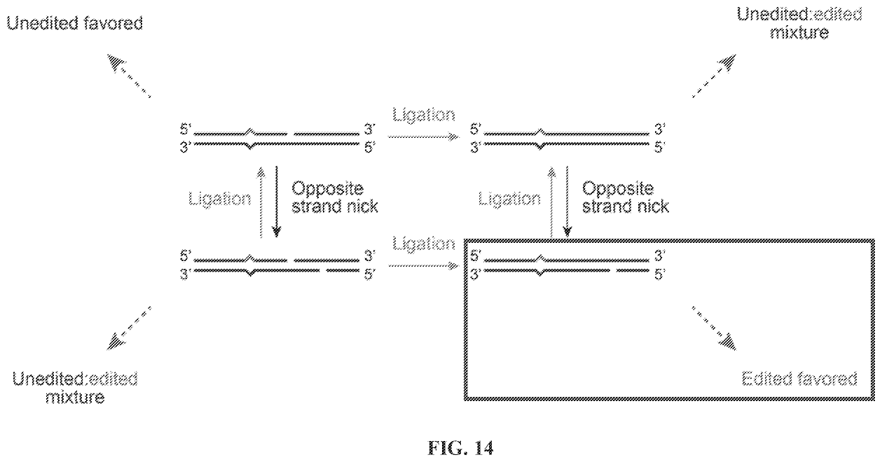

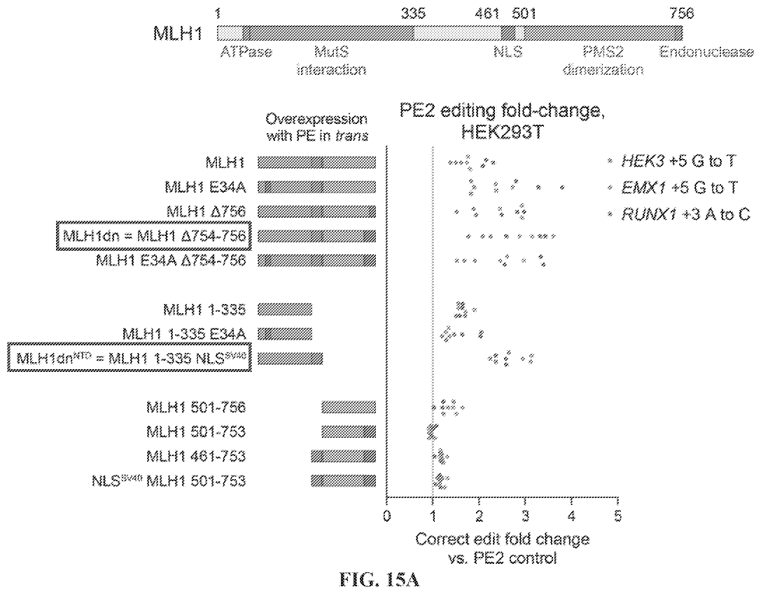

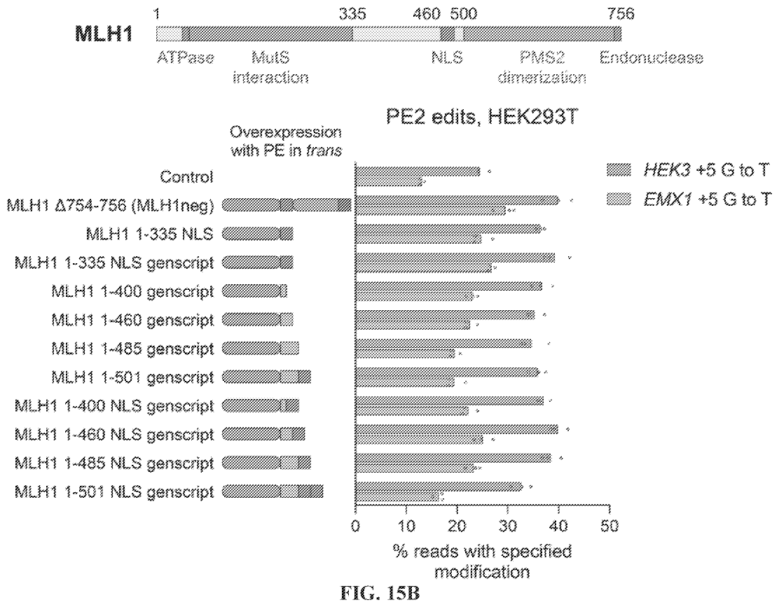

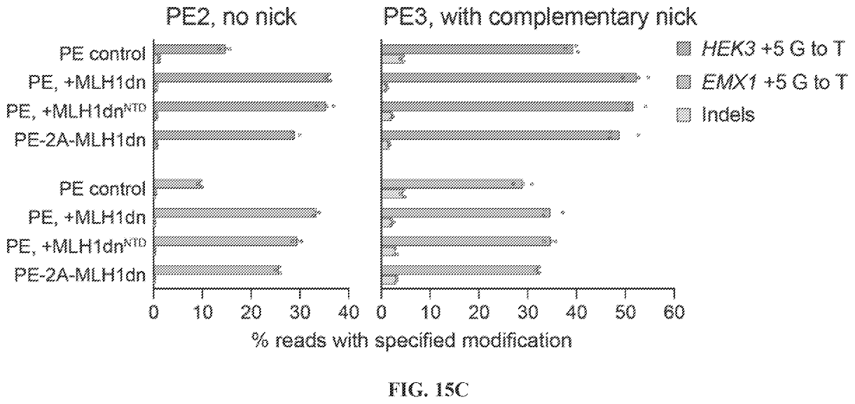

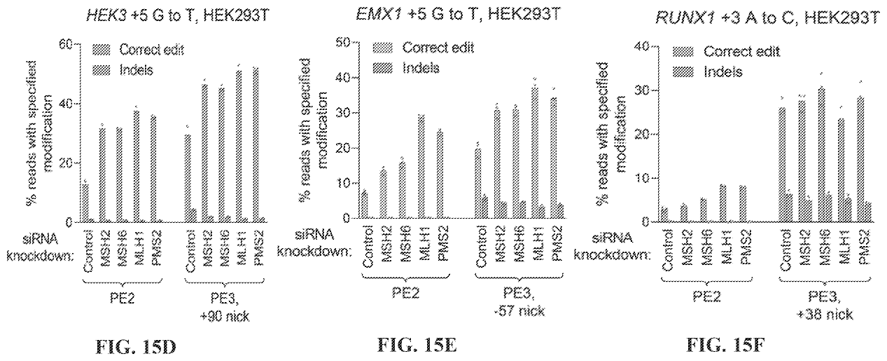

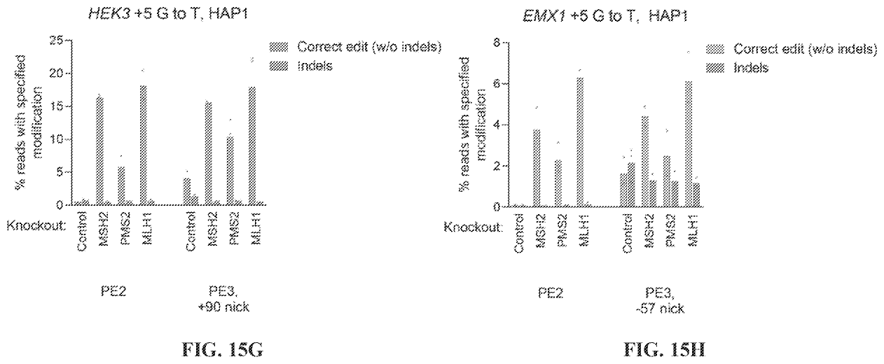

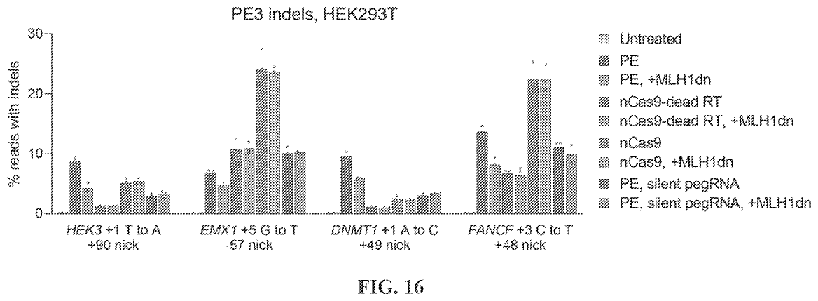

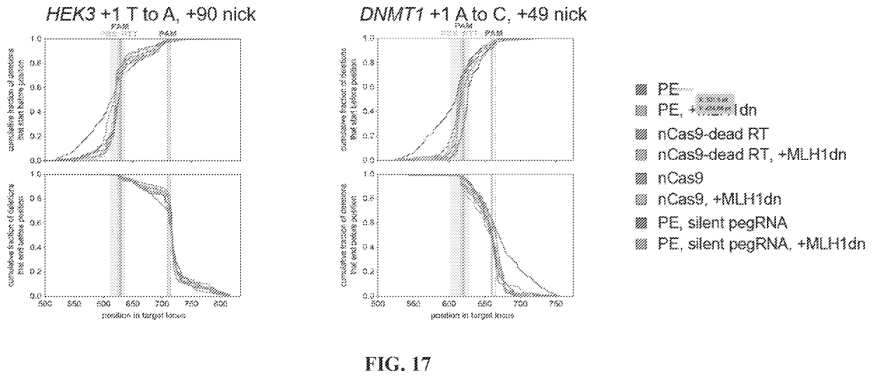

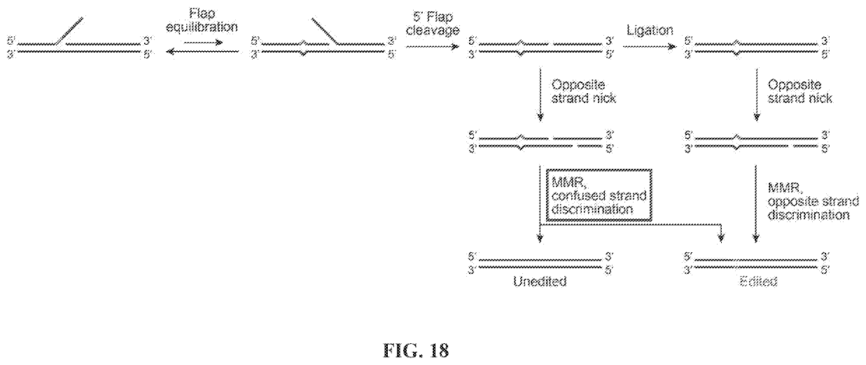

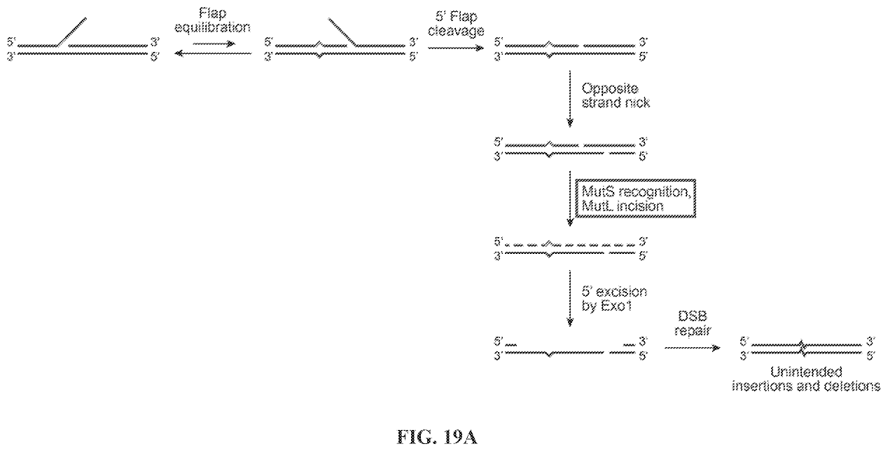

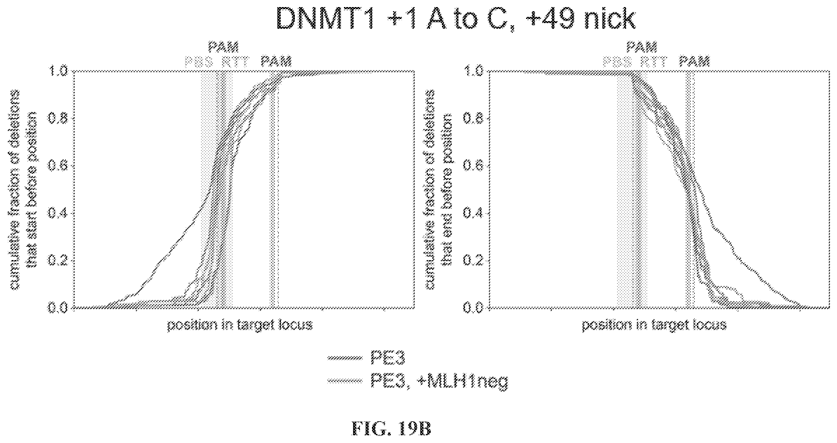

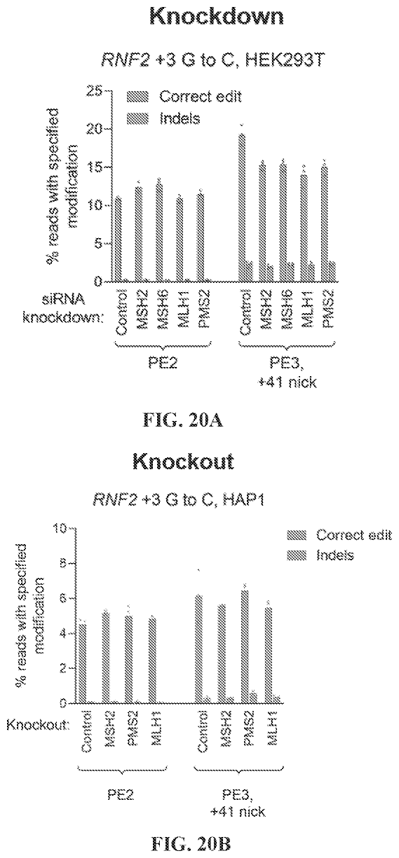

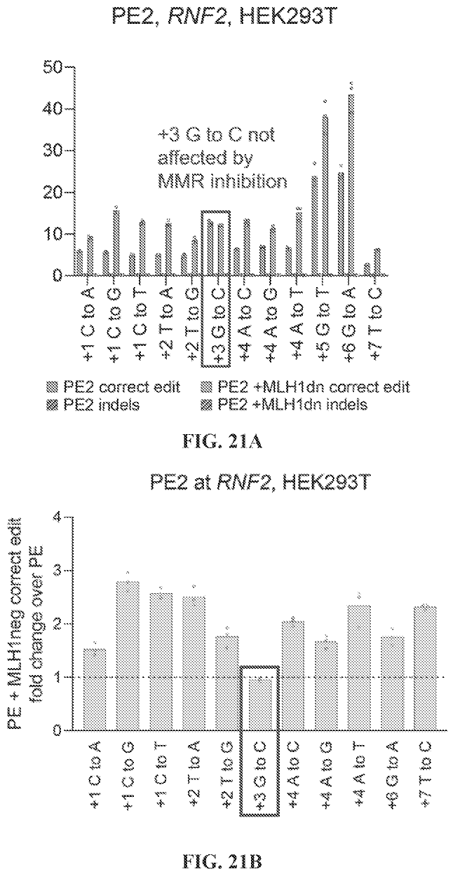

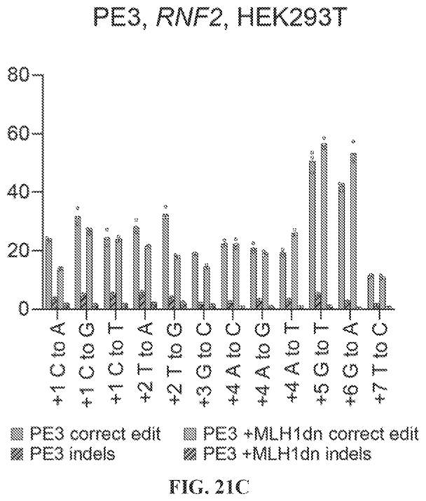

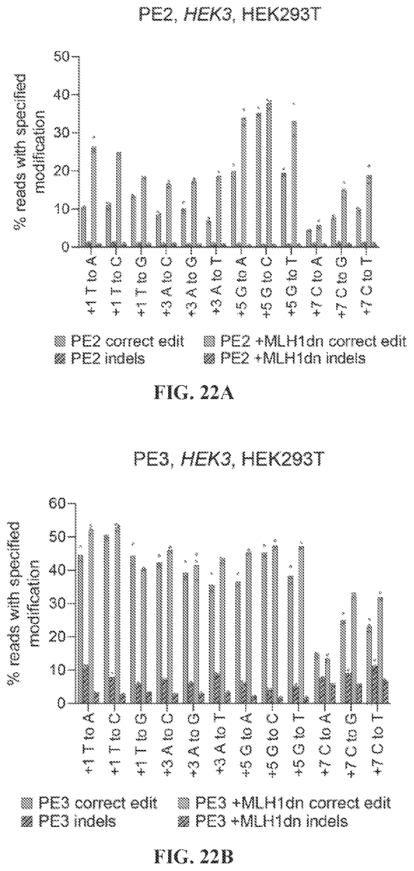

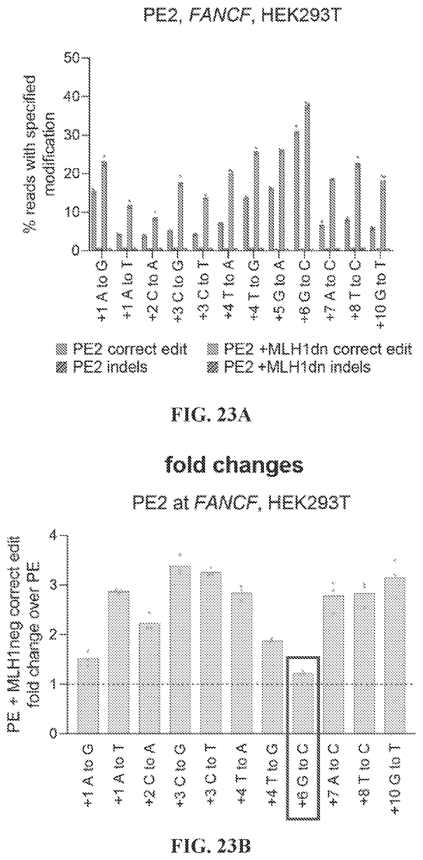

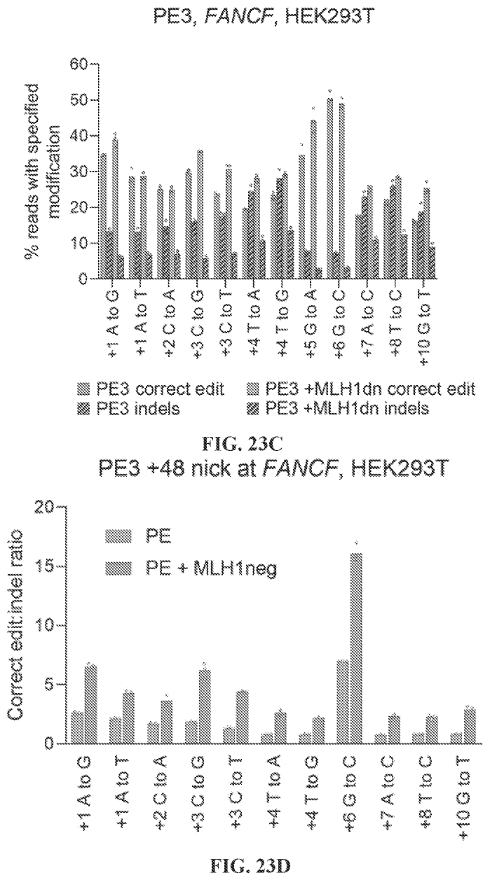

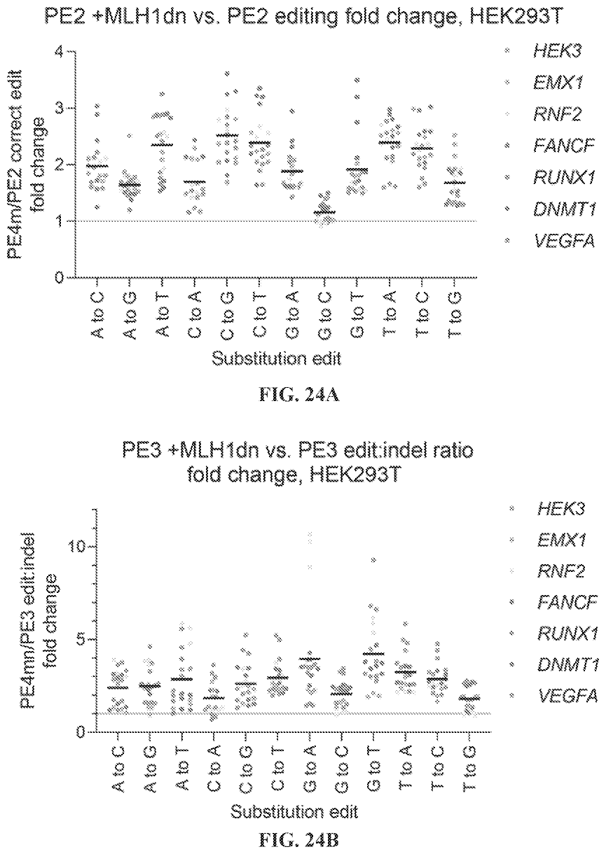

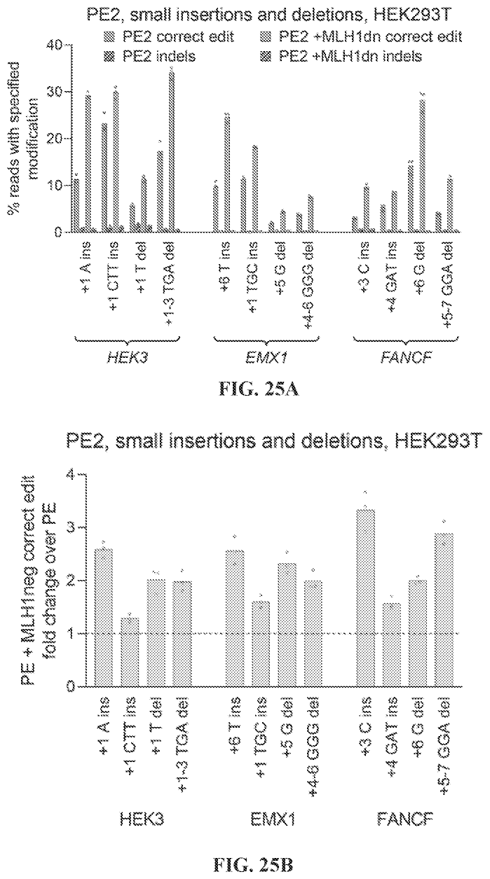

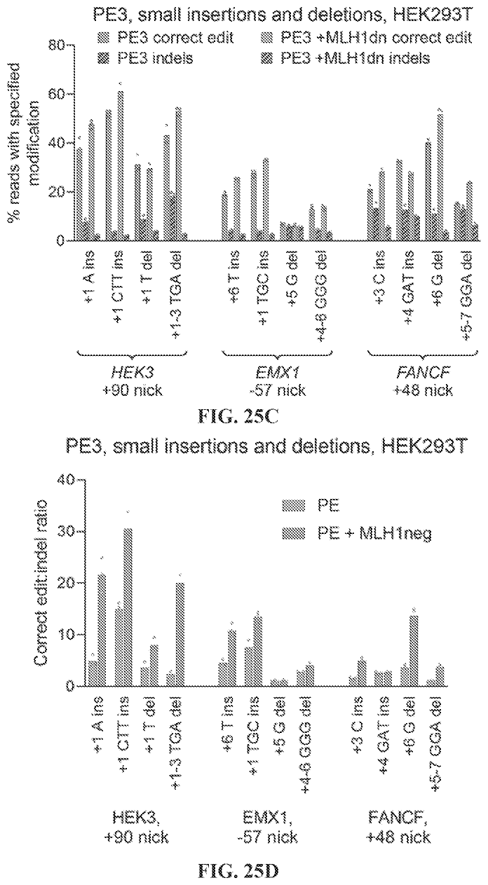

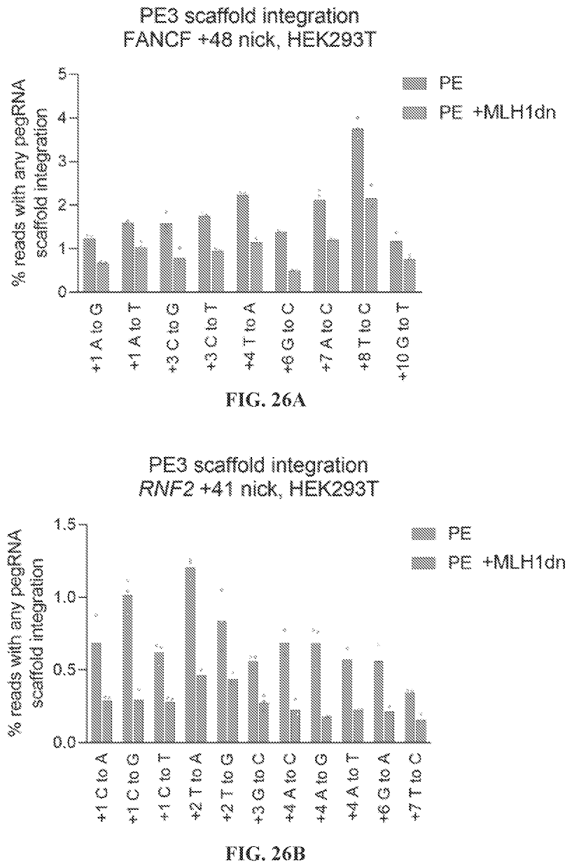

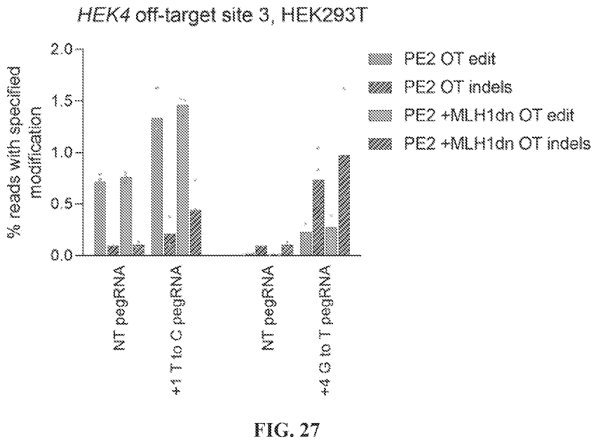

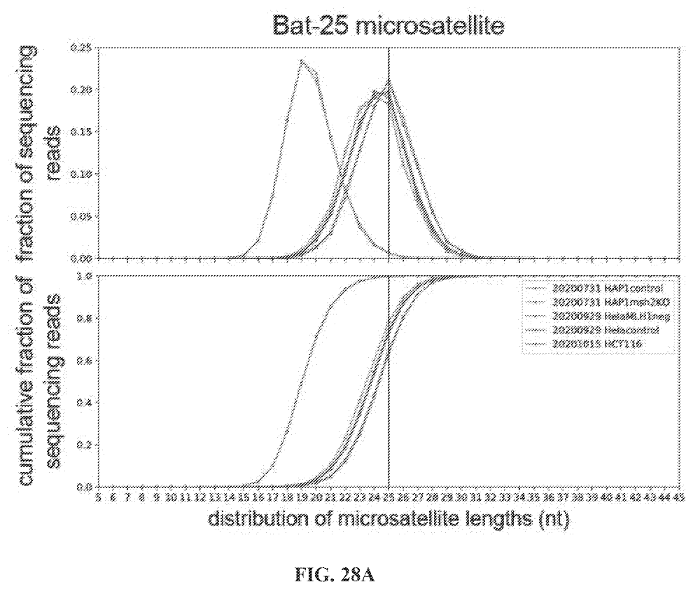

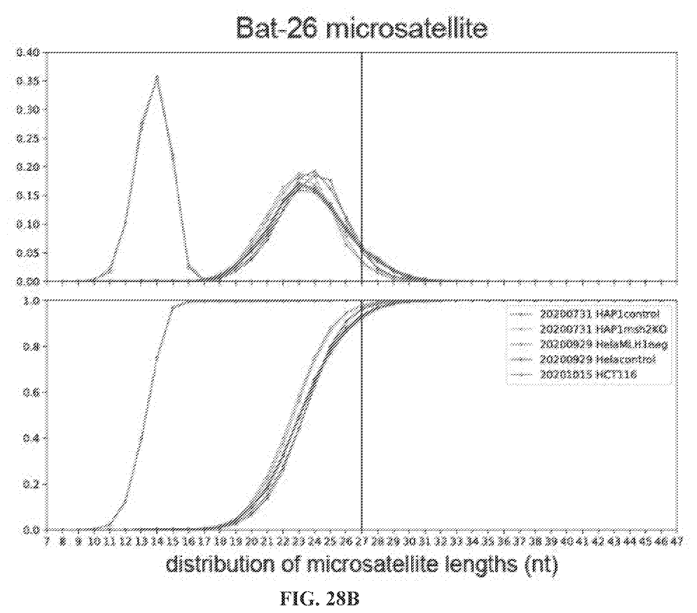

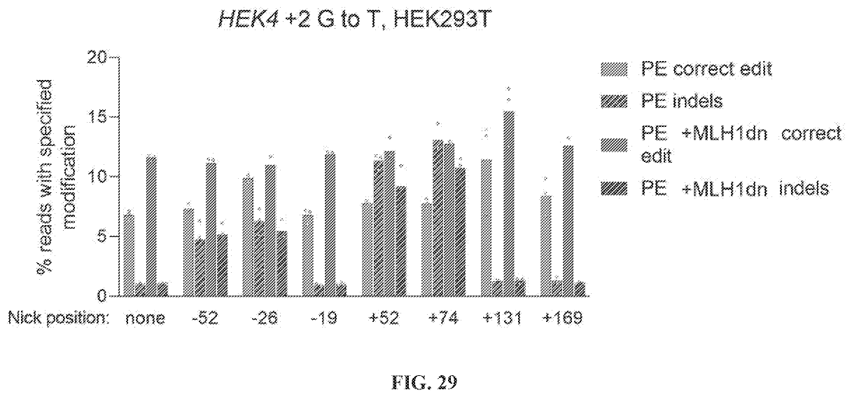

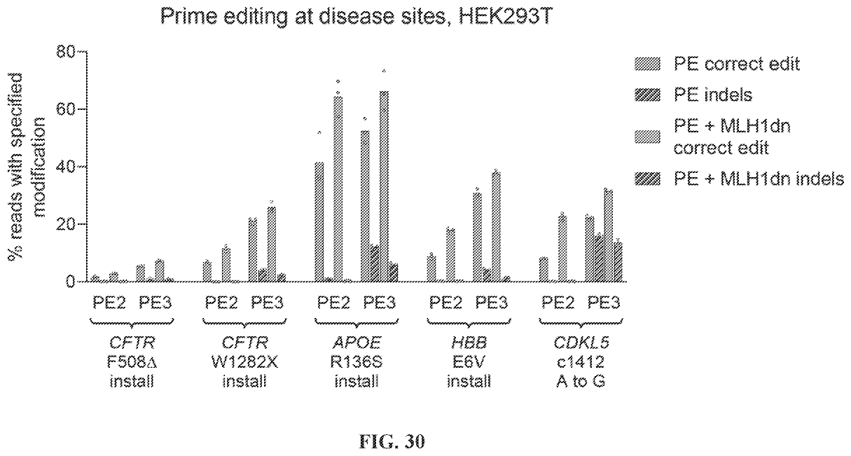

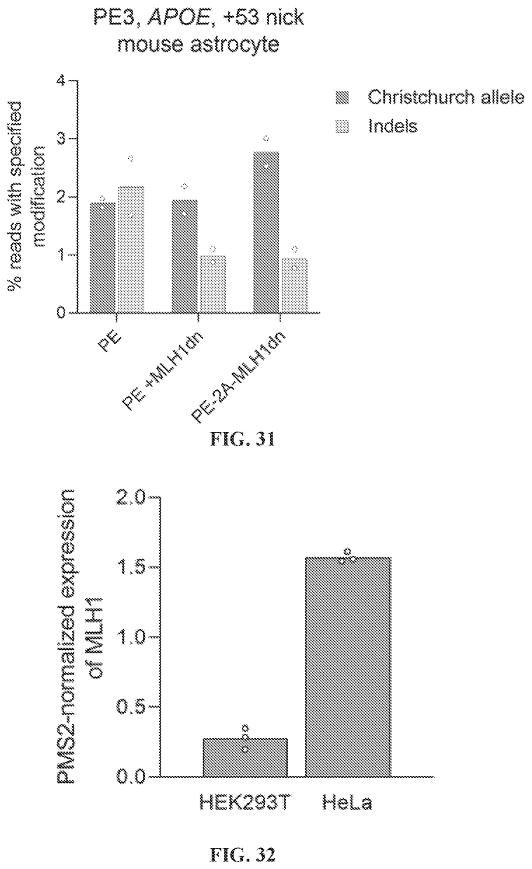

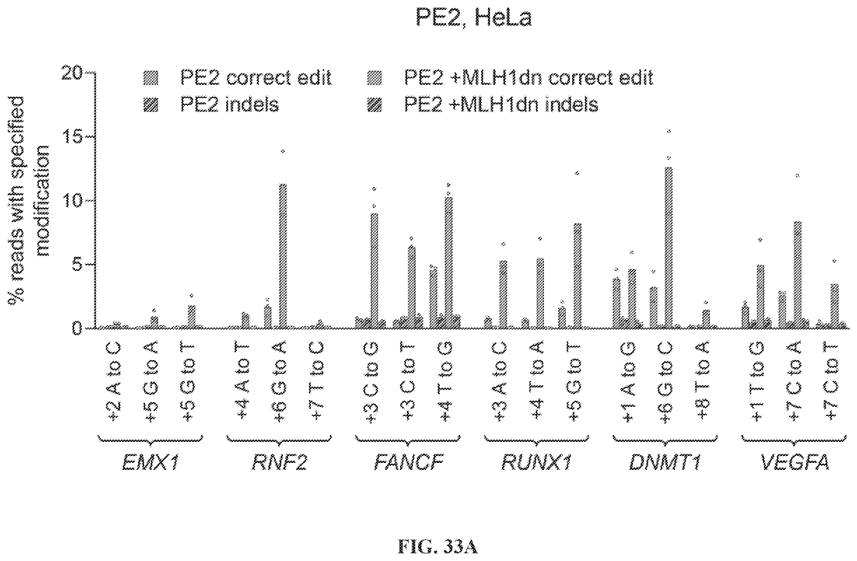

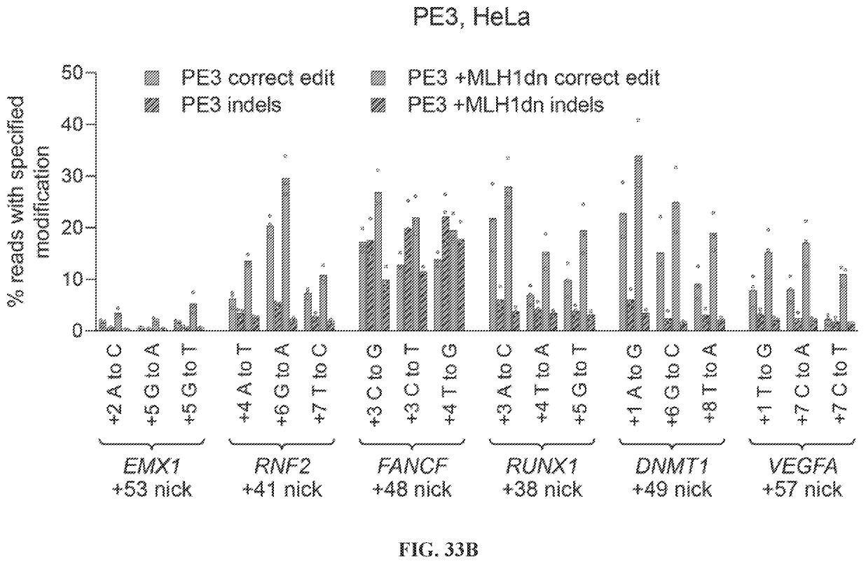

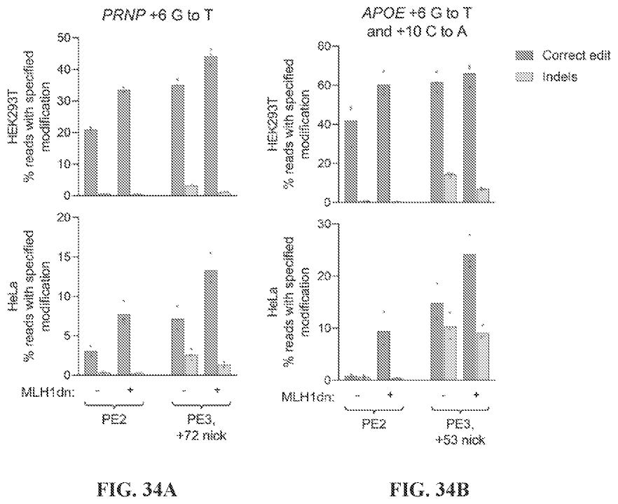

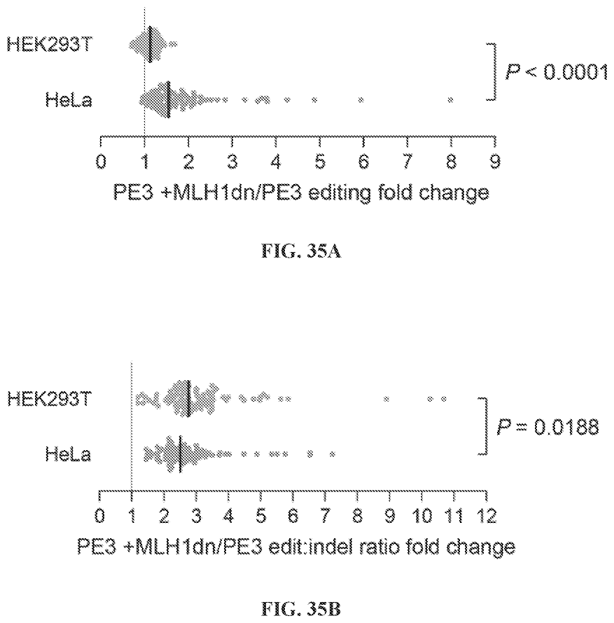

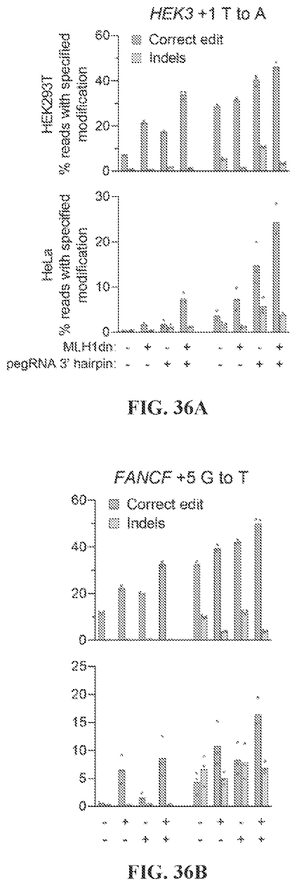

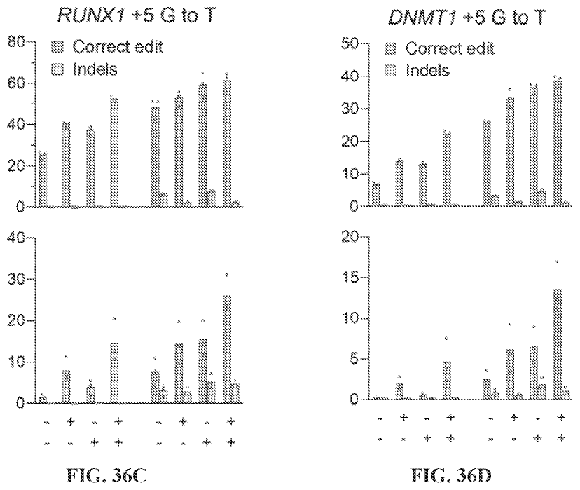

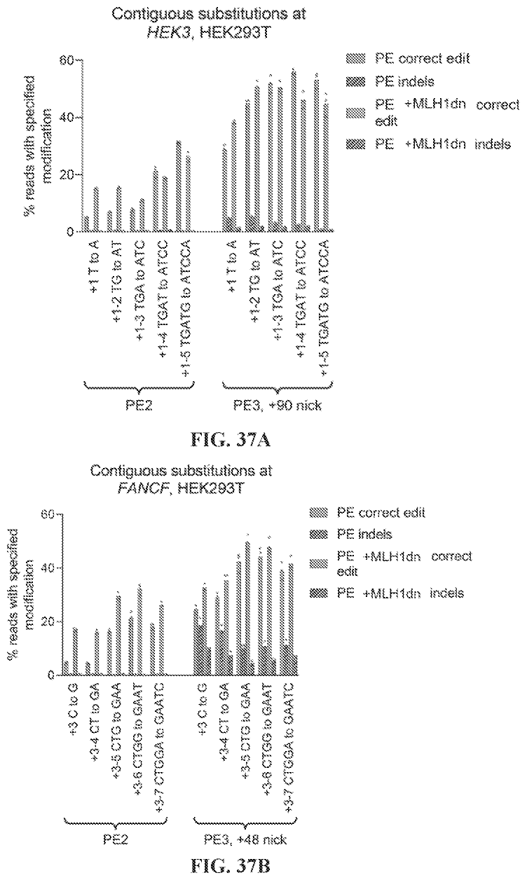

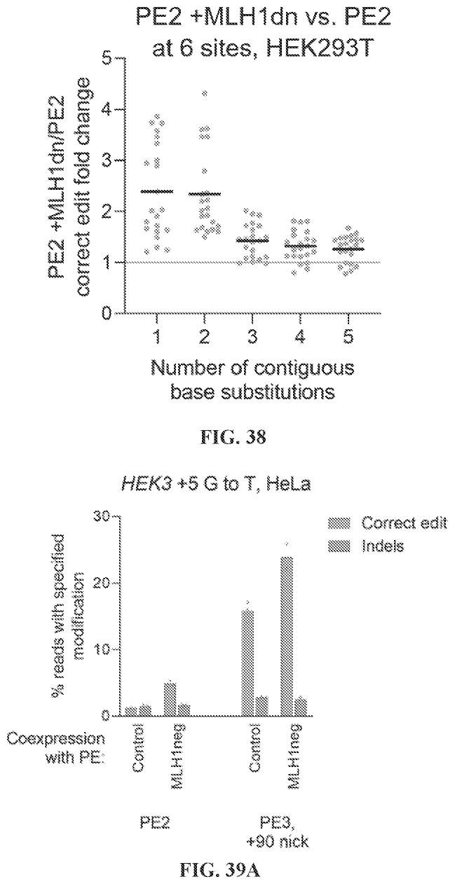

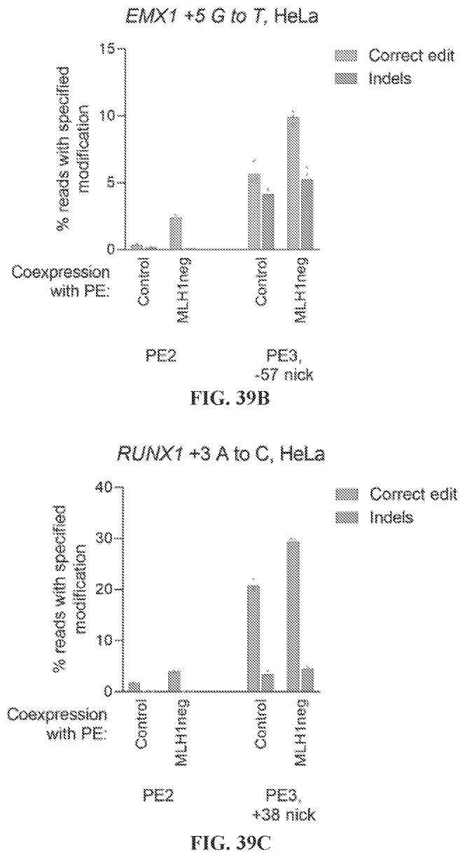

As noted above, the present disclosure relates to the observation that the efficiency and/or specificity of prime editing is impacted by a cell's own DNA mismatch repair (MMR) DNA repair pathway. DNA mismatch repair (MMR) is a highly conserved biological pathway that plays a key role in maintaining genomic stability (e.g., see FIGS. 8A and 8B). Escherichia coli MutS and MutL and their eukaryotic homologs, MutSα and MutLα, respectively, are key players in MMR-associated genome maintenance. In various aspects, the disclosure contemplates any suitable means by which to inhibit, block, or otherwise inactivate the DNA mismatch repair (MMR) system, including, but not limited to inactivating one or more critical proteins of the MMR system at the genetic level, e.g., by introducing one or more mutations in the gene(s) encoding a protein of the MMR system. Such proteins include, but are not limited to MLH1, PMS2 (or MutL alpha), PMS1 (or MutL beta). MLH3 (or MutL gamma), MutS alpha (MSH2-MSH6), MutS beta (MSH2-MSH3), MSH2, MSH6, PCNA, RFC, EXO1, POLδ, and PCNA. The nucleotide and amino acid sequences of such naturally occurring proteins and variants thereof are known in the art. Exemplary sequences are provided herein. The present disclosure embraces using any inhibitor, blocking agent, knockdown strategy, or other means of inactivating any known protein involved in MMR (“MMR protein”), including any wild type or naturally occurring variant of such MMR protein, and any engineered variant (including single or multiple amino acid substitutions, deletions, insertions, rearrangements, or fusions) of such MMR protein, so long as the inhibiting, blocking, or otherwise inactivation of one or more of said MMR proteins or variants thereof result in the inhibition, blockage, or inactivation of the MMR pathway. The inhibiting, blocking, or inactivation of any one or more MMR proteins or variants may be by any suitable means applied at the genetic level (e.g., in the gene encoding the one or more MMR proteins, such as introducing a mutation that inactivates the MMR protein or variant thereof), transcriptional level (e.g., by transcript knockdown), translational level (e.g., by blocking translation of one or more MMR proteins from their cognate transcripts), or at the protein level (e.g., administering of an inhibitor (e.g., small molecule, antibody, dominant negative protein variant) or by targeted protein degradation (e.g., PROTAC-based degradation).

In one aspect, the present disclosure provides an improved method of prime editing comprising additionally inhibiting the DNA mismatch repair (MMR) system during prime editing by inhibiting, blocking, or otherwise inactivating MLH1 or a variant thereof.

Without being bound by theory, MLH1 is a key MMR protein that heterodimerizes with PMS2 to form MutL alpha, a component of the post-replicative DNA mismatch repair system (MMR). DNA repair is initiated by MutS alpha (MSH2-MSH6) or MutS beta (MSH2-MSH3) binding to a dsDNA mismatch, then MutL alpha is recruited to the heteroduplex. Assembly of the MutL-MutS-heteroduplex ternary complex in presence of RFC and PCNA is sufficient to activate endonuclease activity of PMS2. It introduces single-strand breaks near the mismatch and thus generates new entry points for the exonuclease EXO1 to degrade the strand containing the mismatch. DNA methylation would prevent cleavage and therefore assure that only the newly mutated DNA strand is going to be corrected. MutL alpha (MLH1-PMS2) interacts physically with the clamp loader subunits of DNA polymerase III, suggesting that it may play a role to recruit the DNA polymerase III to the site of the MMR. Also implicated in DNA damage signaling, a process which induces cell cycle arrest and can lead to apoptosis in case of major DNA damages. MLH1 also heterodimerizes with MLH3 to form MutL gamma which plays a role in meiosis.

The “canonical” human MLH1 amino acid sequence is represented by SEQ ID NO: 204.

MLH1 also may include other human isoforms, including P40692-2 (SEQ ID NO: 205), which differs from the canonical sequence in that residues 1-241 of the canonical sequence are missing.

MLH1 also may include a third known isoform known as P40692-3 (SEQ ID NO: 207), which differs from the canonical sequence in that residues 1-101 (of MSFVAGVIRR . . . ASISTYGFRG (SEQ ID NO: 206)) are replaced with MAF.

### MMR Inhibitors and Methods of MMR Inhibition

The present disclosure provides a method for editing a nucleic acid molecule by prime editing that involves contacting a nucleic acid molecule with a prime editor, a pegRNA, and an inhibitor of the DNA mismatch repair pathway, thereby installing one or more modifications to the nucleic acid molecule at a target site with increased editing efficiency and/or lower indel formation. Thus, the present disclosure contemplates any suitable means to inhibit MMR. In one embodiment, the disclosure embraces administering an effective amount of an inhibitor of the MMR pathway. In various embodiments, the MMR pathway may be inhibited by inhibiting, blocking, or inactivating any one or more MMR proteins or variants at the genetic level (e.g., in the gene encoding the one or more MMR proteins, such as introducing a mutation that inactivates the MMR protein or variant thereof), transcriptional level (e.g., by transcript knockdown), translational level (e.g., by blocking translation of one or more MMR proteins from their cognate transcripts), or at the protein level (e.g., application of an inhibitor (e.g., small molecule, antibody, dominant negative protein partner) or by targeted protein degradation (e.g., PROTAC-based degradation). The present disclosure also contemplates methods of prime editing which are designed to install modifications to a nucleic acid molecule that evade correction by the MMR pathway, without the need to provide an MMR inhibitor.

The inventors developed prime editing which enables the insertion, deletion, or replacement of genomic DNA sequences without requiring error-prone double-strand DNA breaks. The present disclosure now provides an improved method of prime editing involving the blocking, inhibiting, or inactivation of the MMR pathway (e.g., by inhibiting, blocking, or inactivating an MMR pathway protein, including MLH1) during prime editing, whereby doing so surprisingly results in increased editing efficiency and reduced indel formation. As used herein, “during” prime editing can embrace any suitable sequence of events, such that the prime editing step can be applied before, at the same time, or after the step of blocking, inhibiting, or inactivating the MMR pathway (e.g., by targeting the inhibition of MLH1).

Prime editing uses an engineered Cas9 nickase-reverse transcriptase fusion protein (e.g., PE1 or PE2) paired with an engineered prime editing guide RNA (pegRNA) that both directs Cas9 to the target genomic site and encodes the information for installing the desired edit. Prime editing proceeds through a multi-step editing process: 1) the Cas9 domain binds and nicks the target genomic DNA site, which is specified by the pegRNA's spacer sequence; 2) the reverse transcriptase domain uses the nicked genomic DNA as a primer to initiate the synthesis of an edited DNA strand using an engineered extension on the pegRNA as a template for reverse transcription—this generates a single-stranded 3′ flap containing the edited DNA sequence; 3) cellular DNA repair resolves the 3′ flap intermediate by the displacement of a 5′ flap species that occurs via invasion by the edited 3′ flap, excision of the 5′ flap containing the original DNA sequence, and ligation of the new 3′ flap to incorporate the edited DNA strand, forming a heteroduplex of one edited and one unedited strand; and 4) cellular DNA repair replaces the unedited strand within the heteroduplex using the edited strand as a template for repair, completing the editing process.

Efficient incorporation of the desired edit requires that the newly synthesized 3′ flap contains a portion of sequence that is homologous to the genomic DNA site. This homology enables the edited 3′ flap to compete with the endogenous DNA strand (the corresponding 5′ flap) for incorporation into the DNA duplex. Because the edited 3′ flap will contain less sequence homology than the endogenous 5′ flap, the competition is expected to favor the 5′ flap strand. Thus, a potential limiting factor in the efficiency of prime editing may be the failure of the 3′ flap, which contains the edit, to effectively invade and displace the 5′ flap strand. Moreover, successful 3′ flap invasion and removal of the 5′ flap only incorporates the edit on one strand of the double-stranded DNA genome. Permanent installation of the edit requires cellular DNA repair to replace the unedited complementary DNA strand using the edited strand as a template. While the cell can be made to favor replacement of the unedited strand over the edited strand (step 4 above) by the introduction of a nick in the unedited strand adjacent to the edit using a secondary sgRNA (the PE3 system), this process still relies on a second stage of DNA repair.

This disclosure describes a modified approach to prime editing that comprises additionally inhibiting, blocking, or otherwise inactivating the DNA mismatch repair (MMR) system. In some embodiments, an MMR inhibitor is provided to the target nucleic acid along with other components of a prime editing system, for example, an exogenous MMR inhibitor such as an siRNA can be provided to a cell comprising the target nucleic acid. In some embodiments, a prime editing system component, e.g., a pegRNA, is designed to install modifications in the target nucleic acid which evade the MMR system, without the need to provide an inhibitor. In certain embodiments, the DNA mismatch repair (MMR) system can be inhibited, blocked, or otherwise inactivating one or more proteins of the MMR system, including, but not limited to MLH1. PMS2 (or MutL alpha), PMS1 (or MutL beta). MLH3 (or MutL gamma), MutS alpha (MSH2-MSH6). MutS beta (MSH2-MSH3), MSH2, MSH6, PCNA, RFC, EXO1, POLδ, and PCNA. The disclosure contemplates any suitable means by which to inhibit, block, or otherwise inactivate the DNA mismatch repair (MMR) system, including, but not limited to inactivating one or more critical proteins of the MMR system at the genetic level, e.g., by introducing one or more mutations in the genes encoding a protein of the MMR system, e.g., MLH1, PMS2 (or MutL alpha), PMS1 (or MutL beta), MLH3 (or MutL gamma). MutS alpha (MSH2-MSH6), MutS beta (MSH2-MSH3), MSH2, MSH6, PCNA, RFC, EXO1, POLδ, and PCNA.

Thus, in one aspect, the present disclosure provides a method for editing a nucleotide molecule (e.g., a genome) using prime editing while blocking, inhibiting, or otherwise inactivating the DNA mismatch repair (MMR) system.

In another aspect, the present disclosure provides a method for editing a nucleotide molecule (e.g., a genome) using prime editing while blocking, inhibiting, or otherwise inactivating a protein of the MMR system, e.g., MLH1, PMS2 (or MutL alpha), PMS1 (or MutL beta), MLH3 (or MutL gamma), MutS alpha (MSH2-MSH6), MutS beta (MSH2-MSH3), MSH2, MSH6, PCNA, RFC, EXO1, POLδ, and PCNA.

In one aspect, the present disclosure provides an improved method of prime editing comprising additionally inhibiting the DNA mismatch repair (MMR) system during prime editing by inhibiting, blocking, or otherwise inactivating MLH1 or a variant thereof. Without being bound by theory, MLH1 is a key MMR protein that heterodimerizes with PMS2 to form MutL alpha, a component of the post-replicative DNA mismatch repair system (MMR). DNA repair is initiated by MutS alpha (MSH2-MSH6) or MutS beta (MSH2-MSH3) binding to a dsDNA mismatch, then MutL alpha is recruited to the heteroduplex. Assembly of the MutL-MutS-heteroduplex ternary complex in presence of RFC and PCNA is sufficient to activate endonuclease activity of PMS2. It introduces single-strand breaks near the mismatch and thus generates new entry points for the exonuclease EXO1 to degrade the strand containing the mismatch. DNA methylation would prevent cleavage and therefore assure that only the newly mutated DNA strand is going to be corrected. MutL alpha (MLH1-PMS2) interacts physically with the clamp loader subunits of DNA polymerase III, suggesting that it may play a role to recruit the DNA polymerase III to the site of the MMR. Also implicated in DNA damage signaling, a process which induces cell cycle arrest and can lead to apoptosis in case of major DNA damages. MLH1 also heterodimerizes with MLH3 to form MutL gamma which plays a role in meiosis. The “canonical” human MLH1 amino acid sequence is represented by SEQ ID NO: 204

MLH1 also may include other human isoforms, including P40692-2 (SEQ ID NO: 205), which differs from the canonical sequence in that residues 1-241 of the canonical sequence are missing.

MLH1 also may include a third known isoform known as P40692-3 (SEQ ID NO: 207), which differs from the canonical sequence in that residues 1-101 (of MSFVAGVIRR . . . ASISTYGFRG (SEQ ID NO: 206)) are replaced with MAF.

The disclosure contemplates that any of the following MLH1 proteins may be inhibited by an inhibitor, or otherwise blocked or inactivated in order to inhibit the MMR pathway during prime editing. In addition, such exemplary proteins may also be used to engineer or otherwise make a dominant negative variant that may be used as a type of inhibitor when administered in an effective amount which blocks, inactivates, or inhibits the MMR. Without being bound by theory, it is believed that MLH1 dominant negative mutants can saturate binding of MutS. Exemplary MLH1 proteins include the following amino acid sequences, or amino acid sequences having at least 70%, at least 75%, at least 80%, at least 85%, at least 90%, at least 95%, at least 96%, at least 97%, at least 98%, at least 99%, or up to 100% sequence identity with any of the following sequences:

| | | SEQ |
|---|---|---|
| Description | Sequence | ID NO: |
| MLH1 | MSFVAGVIRRLDETVVNRIAAGEVIQRPANAIKEMIENCLDAKSTSIQVIVKEGG | 204 |
| Homo sapiens | LKLIQIQDNGTGIRKEDLDIVCERFTTSKLQSFEDLASISTYGFRGEALASISHVAH | |
| SwissProt | VTITTKTADGKCAYRASYSDGKLKAPPKPCAGNQGTQITVEDLFYNIATRRKAL | |
| Accession No. | KNPSEEYGKILEVVGRYSVHNAGISFSVKKQGETVADVRTLPNASTVDNIRSIFG | |
| P40692 | NAVSRELIEIGCEDKTLAFKMNGYISNANYSVKKCIFLLFINHRLVESTSLRKAIE | |
| Wild type | TVYAAYLPKNTHPFLYLSLEISPQNVDVNVHPTKHEVHFLHEESILERVQQHIES | |
| | KLLGSNSSRMYFTQTLLPGLAGPSGEMVKSTTSLTSSSTSGSSDKVYAHQMVRT | |
| | DSREQKLDAFLQPLSKPLSSQPQAIVTEDKTDISSGRARQQDEEMLELPAPAEVA | |
| | AKNQSLEGDTTKGTSEMSEKRGPTSSNPRKRHREDSDVEMVEDDSRKEMTAAC | |
| | TPRRRIINLTSVLSLQEEINEQGHEVLREMLHNHSFVGCVNPQWALAQHQTKLY | |
| | LLNTTKLSEELFYQILIYDFANFGVLRLSEPAPLFDLAMLALDSPESGWTEEDGP | |
| | KEGLAEYIVEFLKKKAEMLADYFSLEIDEEGNLIGLPLLIDNYVPPLEGLPIFILRL | |
| | ATEVNWDEEKECFESLSKECAMFYSIRKQYISEESTLSGQQSEVPGSIPNSWKWT | |
| | VEHIVYKALRSHILPPKHFTEDGNILQLANLPDLYKVFERC | |
| MLH1 | MAFVAGVIRRLDETVVNRIAAGEVIQRPANAIKEMIENCLDAKSTNIQVVVKEG | 219 |
| Mus musculus | GLKLIQIQDNGTGIRKEDLDIVCERFTTSKLQTFEDLASISTYGFRGEALASISHV | |
| SwissProt | AHVTITTKTADGKCAYRASYSDGKLQAPPKPCAGNQGTLITVEDLFYNIITRRK | |
| Accession No. | ALKNPSEEYGKILEVVGRYSIHNSGISFSVKKQGETVSDVRTLPNATTVDNIRSIF | |
| Q9JK91 | GNAVSRELIEVGCEDKTLAFKMNGYISNANYSVKKCIFLLFINHRLVESAALRK | |
| Wild type | AIETVYAAYLPKNTHPFLYLSLEISPQNVDVNVHPTKHEVHFLHEESILQRVQQH | |
| | IESKLLGSNSSRMYFTQTLLPGLAGPSGEAARPTTGVASSSTSGSGDKVYAYQM | |
| | VRTDSREQKLDAFLQPVSSLGPSQPQDPAPVRGARTEGSPERATREDEEMLALP | |
| | APAEAAAESENLERESLMETSDAAQKAAPTSSPGSSRKRHREDSDVEMVENAS | |
| | GKEMTAACYPRRRIINLTSVLSLQEEISERCHETLREMLRNHSFVGCVNPQWAL | |
| | AQHQTKLYLLNTTKLSEELFYQILIYDFANFGVLRLSEPAPLFDLAMLALDSPES | |
| | GWTEDDGPKEGLAEYIVEFLKKKAEMLADYFSVEIDEEGNLIGLPLLIDSYVPPL | |
| | EGLPIFILRLATEVNWDEEKECFESLSKECAMFYSIRKQYILEESTLSGQQSDMPG | |
| | STSKPWKWTVEHIIYKAFRSHLLPPKHFTEDGNVLQLANLPDLYKVFERC | |
| MLH1 | MSFVAGVIRRLDETVVNRIAAGEVIQRPANAIKEMTENCLDAKSTNIQVIVREG | 220 |
| Rattus | GLKLIQIQDNGTGIRKEDLDIVCERFTTSKLQTFEDLAMISTYGFRGEALASISHV | |
| norvegicus | AHVTITTKTADGKCAYRASYSDGKLQAPPKPCAGNQGTLITVEDLFYNIITRKK | |
| SwissProt | ALKNPSEEYGKILEVVGRYSIHNSGISFSVKKQGETVSDVRTLPNATTVDNIRSIF | |
| Accession No. | GNAVSRELIEVGCEDKTLAFKMNGYISNANYSVKKCIFLLFINHRLVESAALKK | |
| P97679 | AIEAVYAAYLPKNTHPFLYLILEISPQNVDVNVHPTKHEVHFLHEESILERVQQHI | |
| Wild type | ESKLLGSNSSRMYFTQTLLPGLAGPSGEAVKSTTGIASSSTSGSGDKVHAYQMV | |
| | RTDSRDQKLDAFMQPVSRRLPSQPQDPVPGNRTEGSPEKAMQKDQEISELPAPM | |
| | EAAADSASLERESVIGASEVVAPQRHPSSPGSSRKRHPEDSDVEMMENDSRKEM | |
| | TAACYPRRRIINLTSVLSLQEEINDRGHETLREMLRNHTFVGCVNPQWALAQHQ | |
| | TKLYLLNTTKLSEELFYQILIYDFANFGVLRLPEPAPLFDFAMLALDSPESGWTE | |
| | EDGPKEGLAEYIVEFLKKKAKMLADYFSVEIDEEGNLIGLPLLIDSYVPPLEGLPI | |
| | FILRLATEVNWDEEECFESLSKECAVFYSIRKQYILEESALSGQQSDMPGSPSKP | |
| | WKWTVEHIIYKAFRSHLLPPKHFTEDGNVLQLANLPDLCKVFERC | |
| MLH1 | MSLVAGVIRRLDETVVNRIAAGEVIQRPANAIKEMIENCLDAKSTSIQVVVKEG | 221 |
| Bos taurus | GLKLIQIQDNGTGIRKEDLEIVCERFTTSKLQSFEDLAHISTYGFRGEALASISHV | |
| SwissProt | AHVTITTKTADGKCAYRAHYSDGKLKAPPKPCAGNQGTQITVEDLFYNISTRRK | |
| Accession No. | ALKNPSEEYGKILEVVGRYAVHNSGIGFSVKKQGETVADVRTLPNATTVDNIRS | |
| FIMPGO | IFGNAVSRELIEVECEDKTLAFKMNGYISNANYSVKKCIFLLFINHRLVESASLRK | |
| Wild type | AIETVYAAYLPKSTHPFLYLSLEISPQNVDVNVHPTKHEVHFLHEDSILERLQQHI | |
| | ESRLLGSNASRTYFTQTLLPGLPGPSGEAVKSTASVTSSSTAGSGDRVYAHQMV | |
| | RTDCREQKLDAFLQPVSKALSSQPQAVVPEHRTDASSSGTRQQDEEMLELPAPA | |
| | AVAAKSQALEDDATMRAADLAEKRGPSSSPENPRKRPREDSDVEMVEDASRKE | |
| | MTAACTPRRRIINLTSVLSLQEEINERGHETLREMLHNHSFVGCVNPQWALAQH | |
| | QTKLYLLNTTRLSEELFYQILVYDFANFGVLRLSEPAPLFDLAMLALDSPESGWT | |
| | EEDGPKEGLAEYIVEFLKKKAEMLADYFSLEIDEEGNLVGLPLLIDNYVPPLEGL | |
| | PIFILRLATEVNWDEEKECFESLSKECAMFYSIRKQYVSAESTLSGQQSEVPGST | |
| | ANPWKWTVEHVIYKAFRSHLLPPKHFTEDGNILQLANLPDLYKVFERC | |

methods and compositions described herein utilize MLH1 mutants or truncated variants. In some embodiments, the mutants and truncated variants of the human MLH1 wild-type protein are utilized.

In one aspect, a truncated variant of human MLH1 is provided by this disclosure. In some embodiments, amino acids 754-756 of the wild-type human MLH1l protein are truncated (Δ754-756, hereinafter referred to as MLH1dn). In some embodiments, a truncated variant of human MLH1 comprising only the N-terminal domain (amino acids 1-335) is provided (hereinafter referred to as MLH1dn^{NTD}. In various embodiments, the following MLH11 variants are provided in this disclosure:

| | | SEQ |
|---|---|---|
| Description | Sequence | ID NO: |
| MLH1 E34A | MSFVAGVIRRLDETVVNRIAAGEVIQRPANAIKAMIENCLDAKSTSIQVIVKEGG | 222 |
| | LKLIQIQDNGTGIRKEDLDIVCERFTTSKLQSFEDLASISTYGFRGEALASISHVAH | |
| | VTITTKTADGKCAYRASYSDGKLKAPPKPCAGNQGTQITVEDLFYNIATRRKAL | |
| | KNPSEEYGKILEVVGRYSVHNAGISFSVKKQGETVADVRTLPNASTVDNIRSIFG | |
| | NAVSRELIEIGCEDKTLAFKMNGYISNANYSVKKCIFLLFINHRLVESTSLRKAIE | |
| | TVYAAYLPKNTHPFLYLSLEISPQNVDVNVHPTKHEVHFLHEESILERVQQHIES | |
| | KLLGSNSSRMYFTQTLLPGLAGPSGEMVKSTTSLTSSSTSGSSDKVYAHQMVRT | |
| | DSREQKLDAFLQPLSKPLSSQPQAIVTEDKTDISSGRARQQDEEMLELPAPAEVA | |
| | AKNQSLEGDTTKGTSEMSEKRGPTSSNPRKRHREDSDVEMVEDDSRKEMTAAC | |
| | TPRRRIINLTSVLSLQEEINEQGHEVLREMLHNHSFVGCVNPQWALAQHQTKLY | |
| | LLNTTKLSEELFYQILIYDFANFGVLRLSEPAPLFDLAMLALDSPESGWTEEDGP | |
| | KEGLAEYIVEFLKKKAEMLADYFSLEIDEEGNLIGLPLLIDNYVPPLEGLPIFILRL | |
| | ATEVNWDEEKECFESLSKECAMFYSIRKQYISEESTLSGQQSEVPGSIPNSWKWT | |
| | VEHIVYKALRSHILPPKHFTEDGNILQLANLPDLYKVFERC | |
| MLH1 Δ756 | MSFVAGVIRRLDETVVNRIAAGEVIQRPANAIKEMIENCLDAKSTSIQVIVKEGG | 208 |
| | LKLIQIQDNGTGIRKEDLDIVCERFTTSKLQSFEDLASISTYGFRGEALASISHVAH | |
| | VTITTKTADGKCAYRASYSDGKLKAPPKPCAGNQGTQITVEDLFYNIATRRKAL | |
| | KNPSEEYGKILEVVGRYSVHNAGISFSVKKQGETVADVRTLPNASTVDNIRSIFG | |
| | NAVSRELIEIGCEDKTLAFKMNGYISNANYSVKKCIFLLFINHRLVESTSLRKAIE | |
| | TVYAAYLPKNTHPFLYLSLEISPQNVDVNVHPTKHEVHFLHEESILERVQQHIES | |
| | KLLGSNSSRMYFTQTLLPGLAGPSGEMVKSTTSLTSSSTSGSSDKVYAHQMVRT | |
| | DSREQKLDAFLQPLSKPLSSQPQAIVTEDKTDISSGRARQQDEEMLELPAPAEVA | |
| | AKNQSLEGDTTKGTSEMSEKRGPTSSNPRKRHREDSDVEMVEDDSRKEMTAAC | |
| | TPRRRIINLTSVLSLQEEINEQGHEVLREMLHNHSFVGCVNPQWALAQHQTKLY | |
| | LLNTTKLSEELFYQILIYDFANFGVLRLSEPAPLFDLAMLALDSPESGWTEEDGP | |
| | KEGLAEYIVEFLKKKAEMLADYFSLEIDEEGNLIGLPLLIDNYVPPLEGLPIFILRL | |
| | ATEVNWDEEKECFESLSKECAMFYSIRKQYISEESTLSGQQSEVPGSIPNSWKWT | |
| | VEHIVYKALRSHILPPKHFTEDGNILQLANLPDLYKVFER[-] | |
| MLH1 Δ754- | MSFVAGVIRRLDETVVNRIAAGEVIQRPANAIKEMIENCLDAKSTSIQVIVKEGG | 209 |
| 756 | LKLIQIQDNGTGIRKEDLDIVCERFTTSKLQSFEDLASISTYGFRGEALASISHVAH | |
| | VTITTKTADGKCAYRASYSDGKLKAPPKPCAGNQGTQITVEDLFYNIATRRKAL | |
| | KNPSEEYGKILEVVGRYSVHNAGISFSVKKQGETVADVRTLPNASTVDNIRSIFG | |
| | NAVSRELIEIGCEDKTLAFKMNGYISNANYSVKKCIFLLFINHRLVESTSLRKAIE | |
| | TVYAAYLPKNTHPFLYLSLEISPQNVDVNVHPTKHEVHFLHEESILERVQQHIES | |
| | KLLGSNSSRMYFTQTLLPGLAGPSGEMVKSTTSLTSSSTSGSSDKVYAHQMVRT | |
| | DSREQKLDAFLQPLSKPLSSQPQAIVTEDKTDISSGRARQQDEEMLELPAPAEVA | |
| | AKNQSLEGDTTKGTSEMSEKRGPTSSNPRKRHREDSDVEMVEDDSRKEMTAAC | |
| | TPRRRIINLTSVLSLQEEINEQGHEVLREMLHNHSFVGCVNPQWALAQHQTKLY | |
| | LLNTTKLSEELFYQILIYDFANFGVLRLSEPAPLFDLAMLALDSPESGWTEEDGP | |
| | KEGLAEYIVEFLKKKAEMLADYFSLEIDEEGNLIGLPLLIDNYVPPLEGLPIFILRL | |
| | ATEVNWDEEKECFESLSKECAMFYSIRKQYISEESTLSGQQSEVPGSIPNSWKWT | |
| | VEHIVYKALRSHILPPKHFTEDGNILQLANLPDLYKVF[---] | |
| MLH1 E34A | MSFVAGVIRRLDETVVNRIAAGEVIQRPANAIKAMIENCLDAKSTSIQVIVKEGG | 210 |
| 4754-756 | LKLIQIQDNGTGIRKEDLDIVCERFTTSKLQSFEDLASISTYGFRGEALASISHVAH | |
| | VTITTKTADGKCAYRASYSDGKLKAPPKPCAGNQGTQITVEDLFYNIATRRKAL | |
| | KNPSEEYGKILEVVGRYSVHNAGISFSVKKQGETVADVRTLPNASTVDNIRSIFG | |
| | NAVSRELIEIGCEDKTLAFKMNGYISNANYSVKKCIFLLFINHRLVESTSLRKAIE | |
| | TVYAAYLPKNTHPFLYLSLEISPQNVDVNVHPTKHEVHFLHEESILERVQQHIES | |
| | KLLGSNSSRMYFTQTLLPGLAGPSGEMVKSTTSLTSSSTSGSSDKVYAHQMVRT | |
| | DSREQKLDAFLQPLSKPLSSQPQAIVTEDKTDISSGRARQQDEEMLELPAPAEVA | |
| | AKNQSLEGDTTKGTSEMSEKRGPTSSNPRKRHREDSDVEMVEDDSRKEMTAAC | |
| | TPRRRIINLTSVLSLQEEINEQGHEVLREMLHNHSFVGCVNPQWALAQHQTKLY | |
| | LLNTTKLSEELFYQILIYDFANFGVLRLSEPAPLFDLAMLALDSPESGWTEEDGP | |
| | KEGLAEYIVEFLKKKAEMLADYFSLEIDEEGNLIGLPLLIDNYVPPLEGLPIFILRL | |
| | ATEVNWDEEKECFESLSKECAMFYSIRKQYISEESTLSGQQSEVPGSIPNSWKWT | |
| | VEHIVYKALRSHILPPKHFTEDGNILQLANLPDLYKVF[---] | |
| MLH1 1-335 | MSFVAGVIRRLDETVVNRIAAGEVIQRPANAIKEMIENCLDAKSTSIQVIVKEGG | 211 |
| | ILKLIQIQDNGTGIRKEDLDIVCERFTTSKLQSFEDLASISTYGFRGEALASISHVAH | |
| | VTITTKTADGKCAYRASYSDGKLKAPPKPCAGNQGTQITVEDLFYNIATRRKAL | |
| | KNPSEEYGKILEVVGRYSVHNAGISFSVKKQGETVADVRTLPNASTVDNIRSIFG | |
| | NAVSRELIEIGCEDKTLAFKMNGYISNANYSVKKCIFLLFINHRLVESTSLRKAIE | |
| | TVYAAYLPKNTHPFLYLSLEISPQNVDVNVHPTKHEVHFLHEESILERVQQHIES | |
| | KLL | |
| MLH1 1-335 | MSFVAGVIRRLDETVVNRIAAGEVIQRPANAIKAMIENCLDAKSTSIQVIVKEGG | 212 |
| E34A | LKLIQIQDNGTGIRKEDLDIVCERFTTSKLQSFEDLASISTYGFRGEALASISHVAH | |
| | VTITTKTADGKCAYRASYSDGKLKAPPKPCAGNQGTQITVEDLFYNIATRRKAL | |
| | KNPSEEYGKILEVVGRYSVHNAGISFSVKKQGETVADVRTLPNASTVDNIRSIFG | |
| | NAVSRELIEIGCEDKTLAFKMNGYISNANYSVKKCIFLLFINHRLVESTSLRKAIE | |
| | TVYAAYLPKNTHPFLYLSLEISPQNVDVNVHPTKHEVHFLHEESILERVQQHIES | |
| | KLL | |
| MLH1 1-335 | MSFVAGVIRRLDETVVNRIAAGEVIQRPANAIKEMIENCLDAKSTSIQVIVKEGG | 213 |
| NIS^{SV40} | LKLIQIQDNGTGIRKEDLDIVCERFTTSKLQSFEDLASISTYGFRGEALASISHVAH | |
| | VTITTKTADGKCAYRASYSDGKLKAPPKPCAGNQGTQITVEDLFYNIATRRKAL | |
| | KNPSEEYGKILEVVGRYSVHNAGISFSVKKQGETVADVRTLPNASTVDNIRSIFG | |
| | NAVSRELIEIGCEDKTLAFKMNGYISNANYSVKKCIFLLFINHRLVESTSLRKAIE | |
| | TVYAAYLPKNTHPFLYLSLEISPQNVDVNVHPTKHEVHFLHEESILERVQQHIES | |
| | KLLPKKKRKV | |
| MLH1 501-756 | INLTSVLSLQEEINEQGHEVLREMLHNHSFVGCVNPQWALAQHQTKLYLLNTT | 215 |
| | KLSEELFYQILIYDFANFGVLRLSEPAPLFDLAMLALDSPESGWTEEDGPKEGLA | |
| | EYIVEFLKKKAEMLADYFSLEIDEEGNLIGLPLLIDNYVPPLEGLPIFILRLATEVN | |
| | WDEEKECFESLSKECAMFYSIRKQYISEESTLSGQQSEVPGSIPNSWKWTVEHIV | |
| | YKALRSHILPPKHFTEDGNILQLANLPDLYKVFERC | |
| MLH1 501-753 | INLTSVLSLQEEINEQGHEVLREMLHNHSFVGCVNPQWALAQHQTKLYLLNTT | 216 |
| | KLSEELFYQILIYDFANFGVLRLSEPAPLFDLAMLALDSPESGWTEEDGPKEGLA | |
| | EYIVEFLKKKAEMLADYFSLEIDEEGNLIGLPLLIDNYVPPLEGLPIFILRLATEVN | |
| | WDEEKECFESLSKECAMFYSIRKQYISEESTLSGQQSEVPGSIPNSWKWTVEHIV | |
| | YKALRSHILPPKHFTEDGNILQLANLPDLYKVF[---] | |
| MLH1 461-756 | KRGPTSSNPRKRHREDSDVEMVEDDSRKEMTAACTPRRRIINLTSVLSLQEEINE | 217 |
| | QGHEVLREMLHNHSFVGCVNPQWALAQHQTKLYLLNTTKLSEELFYQILIYDF | |
| | ANFQVLRLSEPAPLFDLAMLALDSPESGWTEEDGPKEGLAEYIVEFLKKKAEML | |
| | ADYFSLEIDEEGNLIGLPLLIDNYVPPLEGLPIFILRLATEVNWDEEKECFESLSKE | |
| | CAMFYSIRKQYISEESTLSGQQSEVPGSIPNSWKWTVEHIVYKALRSHILPPKHFT | |
| | EDGNILQLANLPDLYKVFERC | |
| MLH1 461-753 | KRGPTSSNPRKRHREDSDVEMVEDDSRKEMTAACTPRRRIINLTSVLSLQEEINE | 218 |
| | QGHEVLREMLHNHSFVGCVNPQWALAQHQTKLYLLNTTKLSEELFYQILIYDF | |
| | ANFGVLRLSEPAPLEDLAMLALDSPESGWTEEDGPKEGLAEYIVEFLKKKAEML | |
| | ADYFSLEIDEEGNLIGLPLLIDNYVPPLEGLPIFILRLATEVNWDEEKECFESLSKE | |
| | CAMFYSIRKQYISEESTLSGQQSEVPGSIPNSWKWTVEHIVYKALRSHILPPKHFT | |
| | EDGNILQLANLPDLYKVF[---] | |
| NLS^{SV40} MLH1 | PKKKRKVINLTSVLSLQEEINEQGHEVLREMLHNHSFVGCVNPQWALAQHQT | 223 |
| 501-753 | KLYLLNTTKLSEELFYQILIYDFANFGVLRLSEPAPLFDLAMLALDSPESGWTEE | |
| | DGPKEGLAEYIVEFLKKKAEMLADYFSLEIDEEGNLIGLPLLIDNYVPPLEGLPIFI | |
| | LRLATEVNWDEEKECFESLSKECAMFYSIRKQYISEESTLSGQQSEVPGSIPNSW | |
| | KWTVEHIVYKALRSHILPPKHFTEDGNILQLANLPDLYKVF[---] | |
| NLS^{SV40} MLH1 | PKKKRKVKRGPTSSNPRKRHREDSDVEMVEDDSRKEMTAACTPRRRIINLTSV | 224 |
| 461-753 | LSLQEEINEQGHEVLREMLHNHSFVGCVNPQWALAQHQTKLYLLNTTKLSEEL | |
| | FYQILIYDFANFGVLRLSEPAPLFDLAMLALDSPESGWTEEDGPKEGLAEYIVEF | |
| | LKKKAEMLADYFSLEIDEEGNLIGLPLLIDNYVPPLEGLPIFILRLATEVNWDEEK | |
| | ECFESLSKECAMFYSIRKQYISEESTLSGQQSEVPGSIPNSWKWTVEHIVYKALR | |
| | SHILPPKHFTEDGNILQLANLPDLYKVF[---] | |

In another aspect, the present disclosure provides a method for editing a nucleotide molecule (e.g., a genome) using prime editing while blocking, inhibiting, or otherwise inactivating PMS2 (or MutL alpha) or variant thereof.

In yet another aspect, the present disclosure provides a method for editing a nucleotide molecule (e.g., a genome) using prime editing while blocking, inhibiting, or otherwise inactivating PMS1 (or MutL beta) or variant thereof.

In still another aspect, the present disclosure provides a method for editing a nucleotide molecule (e.g., a genome) using prime editing while blocking, inhibiting, or otherwise inactivating MLH3 (or MutL gamma) or variant thereof.

In another aspect, the present disclosure provides a method for editing a nucleotide molecule (e.g., a genome) using prime editing while blocking, inhibiting, or otherwise inactivating MutS alpha (MSH2-MSH6) or variant thereof.

In still another aspect, the present disclosure provides a method for editing a nucleotide molecule (e.g., a genome) using prime editing while blocking, inhibiting, or otherwise inactivating MSH2 or variant thereof.

In another aspect, the present disclosure provides a method for editing a nucleotide molecule (e.g., a genome) using prime editing while blocking, inhibiting, or otherwise inactivating MSH6 or variant thereof.

In yet another aspect, the present disclosure provides a method for editing a nucleotide molecule (e.g., a genome) using prime editing while blocking, inhibiting, or otherwise inactivating PCNA or variant thereof.

In still another aspect, the present disclosure provides a method for editing a nucleotide molecule (e.g., a genome) using prime editing while blocking, inhibiting, or otherwise inactivating RFC or variant thereof.

In another aspect, the present disclosure provides a method for editing a nucleotide molecule (e.g., a genome) using prime editing while blocking, inhibiting, or otherwise inactivating EXO1or variant thereof.

In yet another aspect, the present disclosure provides a method for editing a nucleotide molecule (e.g., a genome) using prime editing while blocking, inhibiting, or otherwise inactivating POLδ or variant thereof.

Exemplary amino acid sequences for these MMR proteins (PMS2 (or MutL alpha), PMS1 (or MutL beta), MLH3 (or MutL gamma), MutS alpha (MSH2-MSH6). MutS beta (MSH2-MSH-3), MSH-2, MSH6, PCNA, RFC, EXO1, POLδ, and PCNA) are as follows:

| | | SEQ |
|---|---|---|
| | | ID |
| Description | Sequence | NQ |
| PMS2 | MERAESSSTEPAKAIKPIDRKSVHQICSGQVVLSLSTAVKELVENSLDAGATNIDL | 225 |
| Homo sapiens | KLKDYGVDLIEVSDNGCGVEEENFEGLTLKHHTSKIQEFADLTQVETFGFRGEAL | |
| SwissProt | SSLCALSDVTISTCHASAKVGTRLMFDHNGKIIQKTPYPRPRGTTVSVQQLFSTLP | |
| Accession No. | VRHKEFQRNIKKEYAKMVQVLHAYCIISAGIRVSCTNQLGQGKRQPVVCTGGSP | |
| P54278 | SIKENIGSVFGQKQLQSLIPFVQLPPSDSVCEEYGLSCSDALHNLFYISGFISQCTHG | |
| Wild type | VGRSSTDRQFFFINRRPCDPAKVCRLVNEVYHMYNRHQYPFVVLNISVDSECVDI | |
| | NVTPDKRQILLQEEKLLLAVLKTSLIGMFDSDVNKLNVSQQPLLDVEGNLIKMH | |
| | AADLEKPMVEKQDQSPSLRTGEEKKDVSISRLREAFSLRHTTENKPHSPKTPEPR | |
| | RSPLGQKRGMLSSSTSGAISDKGVLRPQKEAVSSSHGPSDPTDRAEVEKDSGHGS | |
| | TSVDSEGFSIPDTGSHCSSEYAASSPGDRGSQEHVDSQEKAPKTDDSFSDVDCHS | |
| | NQEDTGCKFRVLPQPTNLATPNTKRFKKEEILSSSDICQKLVNTQDMSASQVDVA | |
| | VKINKKVVPLDESMSSLAKRIKQLHHEAQQSEGEQNYRKFRAKICPGENQAAED | |
| | ELRKEISKTMFAEMEIIGQFNLGFIITKLNEDIFIVDQHATDEKYNFEMLQQHTVL | |
| | QGQRLIAPQTLNLTAVNEAVLIENLEIFRKNGFDFVIDENAPVTERAKLISLPTSKN | |
| | WTFGPQDVDELIFMLSDSPGVMCRPSRVKQMFASRACRKSVMIGTALNTSEMK | |
| | KLITHMGEMDHPWNCPHGRPTMRHIANLGVISQN | |
| PMS1 | MKQLPAATVRLLSSSQIITSVVSVVKELIENSLDAGATSVDVKLENYGFDKIEVRD | 226 |
| Homo sapiens | NGEGIKAVDAPVMAMKYYTSKINSHEDLENLTTYGFRGEALGSICCIAEVLITTR | |
| SwissProt | TAADNFSTQYVLDGSGHILSQKPSHLGQGTTVTALRLFKNLPVRKQFYSTAKKC | |
| Accession No. | KDEIKKIQDLLMSFGILKPDLRIVFVHNKAVIWQKSRVSDHKMALMSVLGTAVM | |
| P54277 | NNMESFQYHSEESQIYLSGFLPKCDADHSFTSLSTPERSFIFINSRPVHQKDILKLIR | |
| Wild type | HHYNLKCLKESTRLYPVFFLKIDVPTADVDVNLTPDKSQVLLQNKESVLIALENL | |
| | MTTCYGPLPSTNSYENNKTDVSAADIVLSKTAETDVLFNKVESSGKNYSNVDTS | |
| | VIPFQNDMHNDESGKNTDDCLNHQISIGDFGYGHCSSEISNIDKNTKNAFQDISMS | |
| | NVSWENSQTEYSKTCFISSVKHTQSENGNKDHIDESGENEEEAGLENSSEISADE | |
| | WSRGNILKNSVGENIEPVKILVPEKSLPCKVSNNNYPIPEQMNLNEDSCNKKSNVI | |
| | DNKSGKVTAYDLLSNRVIKKPMSASALFVQDHRPQFLIENPKTSLEDATLQIEEL | |
| | WKTLSEEEKLKYEEKATKDLERYNSQMKRAIEQESQMSLKDGRKKIKPTSAWN | |
| | LAQKHKLKTSLSNQPKLDELLQSQIEKRRSQNIKMVQIPFSMKNLKINEKKQNKV | |
| | DLEEKDEPCLIHNLRFPDAWLMTSKTEVMLLNPYRVEEALLFKRLLENHKLPAEP | |
| | LEKPIMLTESLFNGSHYLDVLYKMTADDQRYSGSTYLSDPRLTANGFKIKLIPGV | |
| | SITENYLEIEGMANCLPFYGVADLKEILNAILNRNAKEVYECRPRKVISYLEGEAV | |
| | RLSRQLPMYLSKEDIQDIIYRMKHQFGNEIKECVHGRPFFHHLTYLPETT | |
| MLH3 | MIKCLSVEVQAKLRSGLAISSLGQCVEELALNSIDAEAKCVAVRVNMETFQVQVI | 227 |
| Homo sapiens | DNGFGMGSDDVEKVGNRYFTSKCHSVQDLENPRFYGFRGEALANIADMASAVE | |
| SwissProt | ISSKKNRTMKTFVKLFQSGKALKACEADVTRASAGTTVTVYNLFYQLPVRRKC | |
| Accession No. | MDPRLEFEKVRQRIEALSLMHPSISFSLRNDVSGSMVLQLPKTKDVCSRFCQIYGL | |
| Q9UHC1 | GKSQKLREISFKYKEFELSGYISSEAHYNKNMQFLFVNKRLVLRTKLHKLIDFLLR | |
| Wild type | KESIICKPKNGPTSRQMNSSLRHRSTPELYGIYVINVQCQFCEYDVCMEPAKTLIE | |
| | FQNWDTLLFCIQEGVKMFLKQEKLFVELSGEDIKEFSEDNGFSLFDATLQKRVTS | |
| | DERSNFQEACNNILDSYEMFNLQSKAVKRKTTAENVNTQSSRDSEATRKNTNDA | |
| | FLYIYESGGPGHSKMTEPSLQNKDSSCSESKMLEQETIVASEAGENEKHKKSFLE | |
| | HSSLENPCGTSLEMFLSPFQTPCHFEESGQDLEIWKESTTVNGMAANILKNNRIQN | |
| | QPKRFKDATEVGCQPLPFATTLWGVHSAQTEKEKKKESSNCGRRNVFSYGRVKL | |
| | CSTGFITHVVQNEKTKSTETEHSFKNYVRPGPTRAQETFGNRTRHSVETPDIKDL | |
| | ASTLSKESGQLPNKKNCRTNISYGLENEPTATYTMFSAFQEGSKKSQTDCILSDTS | |
| | PSFPWYRHVSNDSRKTDKLIGFSKPIVRKKLSLSSQLGSLEKFKRQYGKVENPLD | |
| | TEVEESNGVTTNLSLQVEPDILLKDKNRLENSDVCKITTMEHSDSDSSCQPASHIL | |
| | NSEKFPFSKDEDCLEQQMPSLRESPMTLKELSLFNRKPLDLEKSSESLASKLSRLK | |
| | GSERETQTMGMMSRFNELPNSDSSRKDSKLCSVLTQDFCMLFNNKHEKTENGVI | |
| | PTSDSATQDNSFNKNSKTHSNSNTTENCVISETPLVLPYNNSKVTGKDSDVLIRAS | |
| | EQQIGSLDSPSGMLMNPVEDATGDQNGICFQSEESKARACSETEESNTCCSDWQR | |
| | HFDVALGRMVYVNKMTGLSTFIAPTEDIQAACTKDLTTVAVDVVLENGSQYRC | |
| | QPFRSDLVLPFLPRARAERTVMRQDNRDTVDDTVSSESLQSLFSEWDNPVFARYP | |
| | EVAVDVSSGQAESLAVKIHNILYPYRFTKGMIHSMQVLQQVDNKFIACLMSTKT | |
| | EENGEAGGNLLVLVDQHAAHERIRLEQLIIDSYEKQQAQGSGRKKLLSSTLIPPLE | |
| | ITVTEEQRRLLWCYHKNLEDLGLEFVFPDTSDSLVLVGKVPLCFVEREANELRRG | |
| | RSTVTKSIVEEFIREQLELLQTTGGIQGTLPLTVQKVLASQACHGAIKFNDGLSLQ | |
| | ESCRLIEALSSCQLPFQCAHGRPSMLPLADIDHLEQEKQIKPNLTKLRKMAQAWR | |
| | LFGKAECDTRQSLQQSMPPCEPP | |
| MSH2 | MAVQPKETLQLESAAEVGFVRFFQGMPEKPTTTVRLFDRGDFYTAHGEDALLAA | 228 |
| Homo sapiens | REVFKTQGVIKYMGPAGAKNLQSVVLSKMNFESFVKDLLLVRQYRVEVYKNRA | |
| SwissProt | GNKASKENDWYLAYKASPGNLSQFEDILFGNNDMSASIGVVGVKMSAVDGQRQ | |
| Accession No. | VGVGYVDSIQRKLGLCEFPDNDQFSNLEALLIQIGPKECVLPGGETAGDMGKLRQ | |
| P43246 | IIQRGGILITERKKADFSTKDIYQDLNRLLKGKKGEQMNSAVLPEMENQVAVSSL | |
| Wild type | SAVIKFLELLSDDSNFGQFELTTFDFSQYMKLDIAAVRALNLFQGSVEDTTGSQSL | |
| | AALLNKCKTPQGQRLVNQWIKQPLMDKNRIEERLNLVEAFVEDAELRQTLQEDL | |
| | LRRFPDLNRLAKKFQRQAANLQDCYRLYQGINQLPNVIQALEKHEGKHQKLLLA | |
| | VFVTPLTDLRSDFSKFQEMIETTLDMDQVENHEFLVKPSFDPNLSELREIMNDLE | |
| | KKMQSTLISAARDLGLDPGKQIKLDSSAQFGYYFRVTCKEEKVLRNNKNFSTVDI | |
| | QKNGVKFTNSKLTSLNEEYTKNKTEYEEAQDAIVKEIVNISSGYVEPMQTLNDVL | |
| | AQLDAVVSFAHVSNGAPVPYVRPAILEKGQGRIILKASRHACVEVQDEIAFIPND | |
| | VYFEKDKQMFHIITGPNMGGKSTYIRQTGVIVLMAQIGCFVPCESAEVSIVDCILA | |
| | RVGAGDSQLKGVSTFMAEMLETASILRSATKDSLIIIDELGRGTSTYDGFGLAWAI | |
| | SEYIATKIGAFCMFATHFHELTALANQIPTVNNLHVTALTTEETLTMLYQVKKGV | |
| | CDQSFGIHVAELANFPKHVIECAKQKALELEEFQYIGESQGYDIMEPAAKKCYLE | |
| | REQGEKIIQEFLSKVKQMPFTEMSEENITIKLKQLKAEVIAKNNSFVNEIISRIKVTT | |
| MSH6 | MSRQSTLYSFFPKSPALSDANKASARASREGGRAAAAPGASPSPGGDAAWSEAG | 229 |
| Homo sapiens | PGPRPLARSASPPKAKNLNGGLRRSVAPAAPTSCDFSPGDLVWAKMEGYPWWP | |
| SwissProt | CLVYNHPFDGTFIREKGKSVRVHVQFFDDSPTRGWVSKRLLKPYTGSKSKEAQK | |
| Accession No. | GGHFYSAKPEILRAMQRADEALNKDKIKRLELAVCDEPSEPEEEEEMEVGTTYV | |
| P52701 | TDKSEEDNEIESEEEVQPKTQGSRRSSRQIKKRRVISDSESDIGGSDVEFKPDTKEE | |
| Wild type | GSSDEISSGVGDSESEGLNSPVKVARKRKRMVTGNGSLKRKSSRKETPSATKQAT | |
| | SISSETKNTLRAFSAPQNSESQAHVSGGGDDSSRPTVWYHETLEWLKEEKRRDEH | |
| | RRRPDHPDFDASTLYVPEDFLNSCTPGMRKWWQIKSQNFDLVICYKVGKFYELY | |
| | HMDALIGVSELGLVFMKGNWAHSGFPEIAFGRYSDSLVQKGYKVARVEQTETPE | |
| | MMEARCRKMAHISKYDRVVRREICRIITKGTQTYSVLEGDPSENYSKYLLSLKEK | |
| | EEDSSGHTRAYGVCFVDTSLGKFFIGQFSDDRHCSRFRTLVAHYPPVQVLFEKGN | |
| | LSKETKTILKSSLSCSLQEGLIPGSQFWDASKTLRTLLEEEYFREKLSDGIGVMLPQ | |
| | VLKGMTSESDSIGLTPGEKSELALSALGGCVFYLKKCLIDQELLSMANFEEYIPLD | |
| | SDTVSTTRSGAIFTKAYQRMVLDAVTLNNLEIFLNGTNGSTEGTLLERVDTCHTP | |
| | FGKRLLKQWLCAPLCNHYAINDRLDAIEDLMVVPDKISEVVELLKKLPDLERLLS | |
| | KIHNVGSPLKSQNHPDSRAIMYEETTYSKKKIIDFLSALEGFKVMCKIIGIMEEVA | |
| | DGFKSKILKQVISLQTKNPEGRFPDLTVELNRWDTAFDHEKARKTGLITPKAGFD | |
| | SDYDQALADIRENEQSLLEYLEKQRNRIGCRTIVYWGIGRNRYQLEIPENFTTRNL | |
| | PEEYELKSTKKGCKRYWTKTIEKKLANLINAEERRDVSLKDCMRRLFYNFDKNY | |
| | KDWQSAVECIAVLDVLLCLANYSRGGDGPMCRPVILLPEDTPPFLELKGSRHPCI | |
| | TKTFFGDDFIPNDILIGCEEEEQENGKAYCVLVTGPNMGGKSTLMRQAGLLAVM | |
| | AQMGCYVPAEVCRLTPIDRVFTRLGASDRIMSGESTFFVELSETASILMHATAHS | |
| | LVLVDELGRGTATFDGTAIANAVVKELAETIKCRTLFSTHYHSLVEDYSQNVAV | |
| | RLGHMACMVENECEDPSQETITFLYKFIKGACPKSYGFNAARLANLPEEVIQKGH | |
| | RKAREFEKMNQSLRLFREVCLASERSTVDAEAVHKLLTLIKEL | |
| PCNA | MFEARLVQGSILKKVLEALKDLINEACWDISSSGVNLQSMDSSHVSLVQLTLRSE | 230 |
| Homo sapiens | GFDTYRCDRNLAMGVNLTSMSKILKCAGNEDIITLRAEDNADTLALVFEAPNQE | |
| SwissProt | KVSDYEMKLMDLDVEQLGIPEQEYSCVVKMPSGEFARICRDLSHIGDAVVISCA | |
| Accession No. | KDGVKFSASGELGNGNIKLSQTSNVDKEEEAVTIEMNEPVQLTFALRYLNFFTKA | |
| P12004 | TPLSSTVTLSMSADVPLVVEYKIADMGHLKYYLAPKIEDEEGS | |
| Wild type | | |
| RFC | MDIRKFFGVIPSGKKLVSETVKKNEKTKSDEETLKAKKGIKEIKVNSSRKEDDFK | 231 |
| Homo sapiens | QKQPSKKKRIIYDSDSESEETLQVKNAKKPPEKLPVSSKPGKISRQDPVTYISETDE | |
| SwissProt | EDDFMCKKAASKSKENGRSTNSHLGTSNMKKNEENTKTKNKPLSPIKLTPTSVL | |
| Accession No. | DYFGTGSVQRSNKKMVASKRKELSQNTDESGLNDEAIAKQLQLDEDAELERQL | |
| P35251 | HEDEEFARTLAMLDEEPKTKKARKDTEAGETFSSVQANLSKAEKHKYPHKVKT | |
| Wild type | AQVSDERKSYSPRKQSKYESSKESQQHSKSSADKIGEVSSPKASSKLAIMKRKEE | |
| | SSYKEIEPVASKRKENAIKLKGETKTPKKTKSSPAKKESVSPEDSEKKRTNYQAY | |
| | RSYLNREGPKALGSKEIPKGAENCLEGLIFVITGVLESIERDEAKSLIERYGGKVTG | |
| | NVSKKTNYLVMGRDSGQSKSDKAAALGTKIIDEDGLLNLIRTMPGKKSKYEIAV | |
| | ETEMKKESKLERTPQKNVQGKRKISPSKKESESKKSRPTSKRDSLAKTIKKETDV | |
| | FWKSLDFKEQVAEETSGDSKARNLADDSSENKVENLLWVDKYKPTSLKTIIGQQ | |
| | GDQSCANKLLRWLRNWQKSSSEDKKHAAKFGKFSGKDDGSSFKAALLSGPPGV | |
| | GKTTTASLVCQELGYSYVELNASDTRSKSSLKAIVAESLNNTSIKGFYSNGAASS | |
| | VSTKHALIMDEVDGMAGNEDRGGIQELIGLIKHTKIPIICMCNDRNHPKIRSLVHY | |
| | CFDLRFQRPRVEQIKGAMMSIAFKEGLKIPPPAMNEIILGANQDIRQVLHNLSMW | |
| | CARSKALTYDQAKADSHRAKKDIKMGPFDVARKVFAAGEETAHMSLVDKSDLF | |
| | FHDYSIAPLFVQENYIHVKPVAAGGDMKKHLMLLSRAADSICDGDLVDSQIRSK | |
| | QNWSLLPAQAIYASVLPGELMRGYMTQFPTFPSWLGKHSSTGKHDRIVQDLALH | |
| | MSLRTYSSKRTVNMDYLSLLRDALVQPLTSQGVDGVQDVVALMDTYYLMKED | |
| | FENIMEISSWGGKPSPFSKLDPKVKAAFTRAYNKEAHLTPYSLQAIKASRHSTSPS | |
| | LDSEYNEELNEDDSQSDEKDQDAIETDAMIKKKTKSSKPSKPEKDKEPRKGKGK | |
| | SSKK | |
| EXO1 | MGIQGLLQFIKEASEPIHVRKYKGQVVAVDTYCWLHKGAIACAEKLAKGEPTDR | 156 |
| Homo sapiens | YVGFCMKFVNMLLSHGIKPILVFDGCTLPSKKEVERSRRERRQANLLKGKQLLR | |
| SwissProt | EGKVSEARECFTRSINITHAMAHKVIKAARSQGVDCLVAPYEADAQLAYLNKAG | |
| Accession No. | IVQAIITEDSDLLAFGCKKVILKMDQFGNGLEIDQARLQMCRQLGDVFTEEKFRY | |
| Q9UQ84 | MCILSGCDYLSSLRGIGLAKACKVLRLANNPDIVKVIKKIGHYLKMNITVPEDYIN | |
| Wild type | GFIRANNTFLYQLVFDPIKRKLIPLNAYEDDVDPETLSYAGQYVDDSIALQIALGN | |
| | KDINTFEQIDDYNPDTAMPAHSRSHSWDDKTCQKSANVSSIWHRNYSPRPESGT | |
| | VSDAPQLKENPSTVGVERVISTKGLNLPRKSSIVKRPRSAELSEDDLLSQYSLSFT | |
| | KKTKKNSSEGNKSLSFSEVFVPDLVNGPINKKSVSTPPRTRNKFATFLQRKNEES | |
| | GAVVVPGTRSRFFCSSDSTDCVSNKVSIQPLDETAVTDKENNLHESEYGDQEGKR | |
| | LVDTDVARNSSDDIPNNHIPGDHIPDKATVFTDEESYSFESSKFTRTISPPTLGTLR | |
| | SCFSWSGGLGDFSRTPSPSPSTALQQFRRKSDSPTSLPENNMSDVSQLKSEESSDD | |
| | ESHPLREEACSSQSQESGEFSLQSSNASKLSQCSSKDSDSEESDCNIKLLDSQSDQT | |
| | SKLRLSHFSKKDTPLRNKVPGLYKSSSADSLSTTKIKPLGPARASGLSKKPASIQK | |
| | RKHHNAENKPGLQIKLNELWKNFGFKKDSEKLPPCKKPLSPVRDNIQLTPEAEED | |
| | IFNKPECGRVQRAIFQ | |
| POLδ | MDGKRRPGPGPGVPPKRARGGLWDDDDAPRPSQFEEDLALMEEMEAEHRLQEQ | 232 |
| Homo sapiens | EEEELQSVLEGVADGQVPPSAIDPRWLRPTPPALDPQTEPLIFQQLEIDHYVGPAQ | |
| SwissProt | PVPGGPPPSRGSVPVLRAFGVTDEGFSVCCHIHGFAPYFYTPAPPGFGPEHMGDL | |
| Accession No | QRELNLAISRDSRGGRELTGPAVLAVELCSRESMFGYHGHGPSPFLRITVALPRLV | |
| P28340 | APARRLLEQGIRVAGLGTPSFAPYEANVDFEIRFMVDTDIVGCNWLELPAGKYAL | |
| Wild type | RLKEKATQCQLEADVLWSDVVSHPPEGPWQRIAPLRVLSFDIECAGRKGIFPEPE | |
| | RDPVIQICSLGLRWGEPEPFLRLALTLRPCAPILGAKVQSYEKEEDLLQAWSTFIRI | |
| | MDPDVITGYNIQNFDLPYLISRAQTLKVQTFPFLGRVAGLCSNIRDSSFQSKQTGR | |
| | RDTKVVSMVGRVQMDMLQVLLREYKLRSYTLNAVSFHFLGEQKEDVQHSIITD | |
| | LQNGNDQTRRRLAVYCLKDAYLPLRLLERLMVLVNAVEMARVTGVPLSYLLSR | |
| | GQQVKVVSQLLRQAMHEGLLMPVVKSEGGEDYTGATVIEPLKGYYDVPIATLD | |
| | FSSLYPSIMMAHNLCYTTLLRPGTAQKLGLTEDQFIRTPTGDEFVKTSVRKGLLP | |
| | QILENLLSARKRAKAELAKETDPLRRQVLDGRQLALKVSANSVYGFTGAQVGKL | |
| | PCLEISQSVTGFGRQMIEKTKQLVESKYTVENGYSTSAKVVYGDTDSVMCRFGV | |
| | SSVAEAMALGREAADWVSGHFPSPIRLEFEKVYFPYLLISKKRYAGLLFSSRPDA | |
| | HDRMDCKGLEAVRRDNCPLVANLVTASLRRLLIDRDPEGAVAHAQDVISDLLCN | |
| | RIDISQLVITKELTRAASDYAGKQAHVELAERMRKRDPGSAPSLGDRVPYVIISAA | |
| | KGVAAYMKSEDPLFVLEHSLPIDTQYYLEQQLAKPLLRIFEPILGEGRAEAVLLR | |
| | GDHTRCKTVLTGKVGGLLAFAKRRNCCIGCRTVLSHQGAVCEFCQPRESELYQK | |
| | EVSHLNALEERFSRLWTQCQRCQGSLHEDVICTSRDCPIFYMRKKVRKDLEDQE | |
| | QLLRRFGPPGPEAW | |

Thus, in one aspect, the present disclosure provides a method for editing a nucleotide molecule (e.g., a genome) using prime editing while blocking, inhibiting, or otherwise inactivating the DNA mismatch repair (MMR) system.

In another aspect, the present disclosure provides a method for editing a nucleotide molecule (e.g., a genome), comprising contacting a target nucleotide molecule with a prime editor and an inhibitor of the MMR system, e.g., an inhibitor of one or more of MLH1. PMS2 (or MutL alpha). PMS1 (or MutL beta), MLH3 (or MutL gamma), MutS alpha (MSH2-MSH6), MutS beta (MSH2-MSH3), MSH2, MSH6, PCNA, RFC, EXO1, POLδ, or PCNA. In various embodiments, the inhibitor can be a small molecule inhibitor. In other embodiments, the inhibitor can be an antibody, e.g., a neutralizing antibody. In still other embodiments, the inhibitor can be a dominant negative mutant of one or more of MLH1, PMS2 (or MutL alpha), PMS1 (or MutL beta), MLH3 (or MutL gamma), MutS alpha (MSH2-MSH6). MutS beta (MSH2-MSH3), MSH2, MSH6, PCNA, RFC, EXO1, POLδ, or PCNA, e.g., a dominant negative mutant of MLH1. In still other embodiments, the inhibitor can be targeted at the level of transcription, e.g., an siRNA or other nucleic acid agent that knocks down the level of a transcript encoding MLH1, PMS2 (or MutL alpha), PMS1 (or MutL beta), MLH3 (or MutL gamma), MutS alpha (MSH2-MSH6), MutS beta (MSH2-MSH3), MSH2, MSH6, PCNA, RFC, EXO1, POLδ, or PCNA. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating MLH1 or variant thereof. In another aspect, the present disclosure provides a method for editing a nucleotide molecule (e.g., a genome), comprising contacting a target nucleotide molecule with a prime editor and an inhibitor of MLH1. In various embodiments, the inhibitor can be a small molecule inhibitor. In other embodiments, the inhibitor can be an anti-MLH1 antibody, e.g., a neutralizing antibody that inactivates MLH1. In still other embodiments, the inhibitor can be a dominant negative mutant of MLH1. In still other embodiments, the inhibitor can be targeted at the level of transcription of MLH1, e.g., an siRNA or other nucleic acid agent that knocks down the level of a transcript encoding MLH1. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating PMS2 (or MutL alpha) or variant thereof. In another aspect, the present disclosure provides a method for editing a nucleotide molecule (e.g., a genome), comprising contacting a target nucleotide molecule with a prime editor and an inhibitor of PMS2 (or MutL alpha). In various embodiments, the inhibitor can be a small molecule inhibitor. In other embodiments, the inhibitor can be an anti-PMS2 (or MutL alpha) antibody, e.g., a neutralizing antibody that inactivates PMS2 (or MutL alpha). In still other embodiments, the inhibitor can be a dominant negative mutant of PMS2 (or MutL alpha). In still other embodiments, the inhibitor can be targeted at the level of transcription of PMS2 (or MutL alpha), e.g., an siRNA or other nucleic acid agent that knocks down the level of a transcript encoding ML PMS2 (or MutL alpha). In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating PMS1 (or MutL beta) or variant thereof. In another aspect, the present disclosure provides a method for editing a nucleotide molecule (e.g., a genome), comprising contacting a target nucleotide molecule with a prime editor and an inhibitor of PMS1 (or MutL beta). In various embodiments, the inhibitor can be a small molecule inhibitor. In other embodiments, the inhibitor can be an anti-PMS1 (or MutL beta) antibody, e.g., a neutralizing antibody that inactivates PMS1 (or MutL beta). In still other embodiments, the inhibitor can be a dominant negative mutant of PMS1 (or MutL beta). In still other embodiments, the inhibitor can be targeted at the level of transcription of PMS1 (or MutL beta), e.g., an siRNA or other nucleic acid agent that knocks down the level of a transcript encoding PMS1 (or MutL beta). In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating MLH3 (or MutL gamma) or variant thereof. In another aspect, the present disclosure provides a method for editing a nucleotide molecule (e.g., a genome), comprising contacting a target nucleotide molecule with a prime editor and an inhibitor of MLH3 (or MutL gamma). In various embodiments, the inhibitor can be a small molecule inhibitor. In other embodiments, the inhibitor can be an anti-MLH3 (or MutL gamma) antibody, e.g., a neutralizing antibody that inactivates MLH3 (or MutL gamma). In still other embodiments, the inhibitor can be a dominant negative mutant of MLH3 (or MutL gamma). In still other embodiments, the inhibitor can be targeted at the level of transcription of P MLH3 (or MutL gamma), e.g., an siRNA or other nucleic acid agent that knocks down the level of a transcript encoding MLH3 (or MutL gamma). In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating MutS alpha (MSH2-MSH6) or variant thereof. In another aspect, the present disclosure provides a method for editing a nucleotide molecule (e.g., a genome), comprising contacting a target nucleotide molecule with a prime editor and an inhibitor of MutS alpha (MSH2-MSH6). In various embodiments, the inhibitor can be a small molecule inhibitor. In other embodiments, the inhibitor can be an anti-MutS alpha (MSH2-MSH6) antibody, e.g., a neutralizing antibody that inactivates MutS alpha (MSH2-MSH6). In still other embodiments, the inhibitor can be a dominant negative mutant of MutS alpha (MSH2-MSH6). In still other embodiments, the inhibitor can be targeted at the level of transcription of MutS alpha (MSH2-MSH6), e.g., an siRNA or other nucleic acid agent that knocks down the level of a transcript encoding MutS alpha (MSH2-MSH6). In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating MSH2 or variant thereof. In another aspect, the present disclosure provides a method for editing a nucleotide molecule (e.g., a genome), comprising contacting a target nucleotide molecule with a prime editor and an inhibitor of MSH2. In various embodiments, the inhibitor can be a small molecule inhibitor. In other embodiments, the inhibitor can be an anti-MSH2 antibody. e.g., a neutralizing antibody that inactivates MSH2. In still other embodiments, the inhibitor can be a dominant negative mutant of MSH2. In still other embodiments, the inhibitor can be targeted at the level of transcription of MSH2, e.g., an siRNA or other nucleic acid agent that knocks down the level of a transcript encoding MSH2. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating MSH6 or variant thereof. In another aspect, the present disclosure provides a method for editing a nucleotide molecule (e.g., a genome), comprising contacting a target nucleotide molecule with a prime editor and an inhibitor of MS H6. In various embodiments, the inhibitor can be a small molecule inhibitor. In other embodiments, the inhibitor can be an anti-MSH6 antibody, e.g., a neutralizing antibody that inactivates MSH6. In still other embodiments, the inhibitor can be a dominant negative mutant of MSH6. In still other embodiments, the inhibitor can be targeted at the level of transcription of MSH6, e.g., an siRNA or other nucleic acid agent that knocks down the level of a transcript encoding MSH6. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating PCNA or variant thereof. In another aspect, the present disclosure provides a method for editing a nucleotide molecule (e.g., a genome), comprising contacting a target nucleotide molecule with a prime editor and an inhibitor of PCNA. In various embodiments, the inhibitor can be a small molecule inhibitor. In other embodiments, the inhibitor can be an anti-PCNA antibody, e.g., a neutralizing antibody that inactivates PCNA. In still other embodiments, the inhibitor can be a dominant negative mutant of PCNA. In still other embodiments, the inhibitor can be targeted at the level of transcription of PCNA, e.g., an siRNA or other nucleic acid agent that knocks down the level of a transcript encoding PCNA. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating RFC or variant thereof. In another aspect, the present disclosure provides a method for editing a nucleotide molecule (e.g., a genome), comprising contacting a target nucleotide molecule with a prime editor and an inhibitor of RFC. In various embodiments, the inhibitor can be a small molecule inhibitor. In other embodiments, the inhibitor can be an anti-RFC antibody, e.g., a neutralizing antibody that inactivates RFC. In still other embodiments, the inhibitor can be a dominant negative mutant of RFC. In still other embodiments, the inhibitor can be targeted at the level of transcription of RFC, e.g., an siRNA or other nucleic acid agent that knocks down the level of a transcript encoding RFC. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating EXO1 or variant thereof. In another aspect, the present disclosure provides a method for editing a nucleotide molecule (e.g., a genome), comprising contacting a target nucleotide molecule with a prime editor and an inhibitor of EXO1. In various embodiments, the inhibitor can be a small molecule inhibitor. In other embodiments, the inhibitor can be an anti-EXO1 antibody, e.g., a neutralizing antibody that inactivates EXO1. In still other embodiments, the inhibitor can be a dominant negative mutant of EXO1. In still other embodiments, the inhibitor can be targeted at the level of transcription of EXO1, e.g., an siRNA or other nucleic acid agent that knocks down the level of a transcript encoding EXO1. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still another aspect, the present disclosure provides a method for editing a nucleotide molecule (e.g., a genome) using prime editing while blocking, inhibiting, or otherwise inactivating POLδ or variant thereof. In another aspect, the present disclosure provides a method for editing a nucleotide molecule (e.g., a genome), comprising contacting a target nucleotide molecule with a prime editor and an inhibitor of POLδ. In various embodiments, the inhibitor can be a small molecule inhibitor. In other embodiments, the inhibitor can be an anti-POLδ antibody, e.g., a neutralizing antibody that inactivates POLδ. In still other embodiments, the inhibitor can be a dominant negative mutant of POLδ. In still other embodiments, the inhibitor can be targeted at the level of transcription of POLδ, e.g., an siRNA or other nucleic acid agent that knocks down the level of a transcript encoding POLδ. In yet other embodiments, the step of “contacting a target nucleotide molecule with a prime editor” can include (i) delivering directly to a cell an effective amount of a prime editor fusion protein (e.g., PE1 or PE2) complexed with a lipid delivery system; (ii) delivery to a cell a mRNA or delivery complex comprising an mRNA that encodes a prime editor fusion protein and/or a suitable pegRNA; and (iii) a DNA vector (e.g., an AAV or lentivirus vector, plasmid, or other nucleic acid delivery vector) that encodes a prime editor fusion protein and/or a suitable pegRNA on one or more DNA vectors.

In still other aspects, the present disclosure provides methods for prime editing whereby correction by the MMR pathway of the alterations introduced into a target nucleic acid molecule is evaded, without the need to provide an inhibitor of the MMR pathway. Surprisingly, pegRNAs designed with consecutive nucleotide mismatches compared to a target site on the target nucleic acid, for example, pegRNAs that have three or more consecutive mismatching nucleotides, can evade correction by the MMR pathway, resulting in an increase in prime editing efficiency and/or a decrease in the frequency of indel formation compared to the introduction of a single nucleotide mismatch using prime editing. In addition, insertions and deletions of multiple consecutive nucleotides, for example, three or more contiguous nucleotides, or 10 or more contiguous nucleotides in length introduced by prime editing may also evade correction by the MMR pathway, resulting in an increase in prime editing efficiency and/or a decrease in the frequency of indel formation compared to prime editing with a corresponding control pegRNA (e.g., a control pegRNA that does not introduce insertion or deletion of three or more contiguous nucleotides). In some embodiments, prime editing that introduces insertion or deletion of 10 or more contiguous nucleotides results in an increase in prime editing efficiency and/or a decrease in indel frequency compared to the introduction of an insertion or deletion of less than 10 nucleotides in length using prime editing.

Thus, in one aspect, the present disclosure provides methods for editing a nucleic acid molecule by prime editing comprising contacting a nucleic acid molecule with a prime editor and a pegRNA comprising a DNA synthesis template on its extension arm comprising three or more consecutive nucleotide mismatches relative to a target site on the nucleic acid molecule. In some embodiments, the pegRNA comprises a DNA synthesis template comprising one or more nucleotide edits compared to the endogenous sequence of the nucleic acid molecule (e.g., a double stranded target DNA) to be edited, wherein the one or more nucleotide edits comprises (i) an intended change is an insertion, deletion, or substitution of x consecutive nucleotides that corrects a mutation (e.g. a disease associated mutation) in the nucleic acid molecule, and (ii) an insertion, deletion, or substitution of y consecutive nucleotides directly adjacent to the x nucleotides, wherein (x+y) is an integer no less than 3. In some embodiments, the insertion, deletion, or substitution of the y consecutive nucleotides is a silent mutation. In some embodiments, the insertion, deletion, or substitution of the y consecutive nucleotides is a benign mutation. The silent mutations may be present in coding regions of the target nucleic acid molecule or in non-coding regions of the target nucleic acid molecule. When the silent mutations are present in a coding region, they introduce into the nucleic acid molecule one or more alternate codons encoding the same amino acid as the unedited nucleic acid molecule. Alternatively, when the silent mutations are in a non-coding region or a junction of a coding region and a non-coding region (e.g., an intron/exon junction), the silent mutations may be present in a region of the nucleic acid molecule that does not influence splicing, gene regulation, RNA lifetime, or other biological properties of the target site on the nucleic acid molecule. A benign mutation may refer to a nucleotide alteration or amino acid alteration that alters the amino acid sequence of the protein or polypeptide encoded by the target nucleic acid sequence, but does not impair or substantively impair expression and/or function of the protein or polypeptide. In some embodiments, x is an integer between 1 and 50. In some embodiments, y is an integer between 1 and 50. In some embodiments, y is an integer no less than 1. In some embodiments, the inclusion of the silent mutation(s) increases the efficiency, reduces unintended indel frequency, and/or improves editing outcome purity by prime editing. As used herein, the term “prime editing outcome purity” may refer to the ratio of intended edit to unintended indels that result from prime editing. In some embodiments, the inclusion of the silent mutation(s) increases the efficiency, reduces unintended indel frequency, and/or improves editing outcome purity by prime editing by at least 1.5-fold, at least 2.0 fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0 fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, or at least 75-fold compared to prime editing with a control pegRNA that does not include the silent mutation(s), e.g., a control pegRNA that only includes the insertion, deletion, or substitution of the x consecutive nucleotides and not the insertion, deletion, or substitution of the y consecutive nucleotides.

In some embodiments, at least one of the three or more consecutive nucleotide mismatches results in an alteration in the amino acid sequence of a protein expressed from the nucleic acid molecule. In some embodiments, more than one of the consecutive nucleotide mismatches results in an alteration in the amino acid sequence of a protein expressed from the nucleic acid molecule. In some embodiments, at least one of the nucleotide mismatches are silent mutations that do not result in an alteration in the amino acid sequence of a protein expressed from the nucleic acid molecule. The silent mutations may be present in coding regions of the target nucleic acid molecule or in non-coding regions of the target nucleic acid molecule. When the silent mutations are present in a coding region, they introduce into the nucleic acid molecule one or more alternate codons encoding the same amino acid as the unedited nucleic acid molecule. Alternatively, when the silent mutations are in a non-coding region, the silent mutations may be present in a region of the nucleic acid molecule that does not influence splicing, gene regulation, RNA lifetime, or other biological properties of the target site on the nucleic acid molecule.

Any number of consecutive nucleotide mismatches of three or more can be used to achieve the benefits of evading correction by the MMR pathway. In some embodiments, the DNA synthesis template of the extension arm on the pegRNA comprises 3, 4, 5, 6, 7, 8, 9, 10, 11, 12, 13, 14, 15, 16, 17, 18, 19, 20, 21, 22, 23, 24, or 25 consecutive nucleotide mismatches relative to the endogenous sequence of a target site in the nucleic acid molecule edited by prime editing. In some embodiments, the DNA synthesis template of the extension arm on the pegRNA comprises 3, 4, or 5 consecutive nucleotide mismatches relative to the endogenous sequence of a target site in the nucleic acid molecule edited by prime editing. In some embodiments, the DNA synthesis template of the extension arm on the pegRNA comprises 6, 7, 8, 9, 10, 11, 12, 13, 14, 15, 16, 17, 18, 19, or 20 consecutive nucleotide mismatches relative to the endogenous sequence of a target site in the nucleic acid molecule edited by prime editing. In some embodiments, the DNA synthesis template of the extension arm on the pegRNA comprises three or more, four or more, five or more, six or more, seven or more, eight or more, nine or more, or ten or more consecutive nucleotide mismatches relative to a target site on the nucleic acid molecule.

In another aspect, the present disclosure provides methods for editing a nucleic acid molecule by prime editing comprising contacting a nucleic acid molecule with a prime editor and a pegRNA comprising a DNA synthesis template on its extension arm comprising an insertion or deletion of 10 or more nucleotides relative to a target site on the nucleic acid molecule. Insertions and deletions of 10 or more nucleotides in length evade correction by the MMR pathway when introduced by prime editing and thus can benefit from the inhibition of the MMR pathway without the need to provide an inhibitor of MMR. Insertions and deletions of any length greater than 10 nucleotides can be used to achieve the benefits of evading correction by the MMR pathway. In some embodiments, the DNA synthesis template comprises an insertion or deletion of 10, 11, 12, 13, 14, 15, 16, 17, 18, 19, 20, 21, 22, 23, 24, or 25 nucleotides relative to the endogenous sequence at a target site of the nucleic acid molecule edited by prime editing. In some embodiments, the DNA synthesis template comprises an insertion or deletion of 11 or more nucleotides, 12 or more nucleotides, 13 or more nucleotides, 14 or more nucleotides, 15 or more nucleotides, 16 or more nucleotides, 17 or more nucleotides, 18 or more nucleotides, 19 or more nucleotides, 20 or more nucleotides, 21 or more nucleotides, 22 or more nucleotides, 23 or more nucleotides, 24 or more nucleotides, or 25 or more nucleotides relative to a target site on a nucleic acid molecule. In certain embodiments, the DNA synthesis template comprises an insertion or deletion of 15 or more nucleotides relative to a target site on the nucleic acid molecule.

The present disclosure provides compositions and methods for prime editing with improved editing efficiency and/or reduced indel formation by inhibiting the DNA mismatch repair pathway while conducting prime editing of a target site. Accordingly, the present disclosure provides a method for editing a nucleic acid molecule by prime editing that involves contacting a nucleic acid molecule with a prime editor, a pegRNA, and an inhibitor of the DNA mismatch repair pathway, thereby installing one or more modifications to the nucleic acid molecule at a target site with increased editing efficiency and/or lower indel formation. The present disclosure further provides polynucleotides for editing a DNA target site by prime editing comprising a nucleic acid sequence encoding a napDNAbp, a polymerase, and an inhibitor of the DNA mismatch repair pathway, wherein the napDNAbp and polymerase is capable in the presence of a pegRNA of installing one or more modifications in the DNA target site with increased editing efficiency and/or lower indel formation. Thus, the methods and compositions described herein utilize prime editors, which may comprise a nucleic acid programmable DNA binding protein (napDNAbp).

Prime Editors: napDNAbp Domain

In one aspect, a napDNAbp of the prime editors described herein can be associated with or complexed with at least one guide nucleic acid (e.g., guide RNA or a PEgRNA), which localizes the napDNAbp to a DNA sequence that comprises a DNA strand (i.e., a target strand) that is complementary to the guide nucleic acid, or a portion thereof (e.g., the spacer of a guide RNA which anneals to the protospacer of the DNA target). In other words, the guide nucleic-acid “programs” the napDNAbp (e.g., Cas9 or equivalent) to localize and bind to complementary sequence of the protospacer in the DNA.

Any suitable napDNAbp may be used in the prime editors utilized in the methods and compositions described herein. In various embodiments, the napDNAbp may be any Class 2 CRISPR-Cas system, including any type II, type V, or type VI CRISPR-Cas enzyme. Given the rapid development of CRISPR-Cas as a tool for genome editing, there have been constant developments in the nomenclature used to describe and/or identify CRISPR-Cas enzymes, such as Cas9 and Cas9 orthologs. This application references CRISPR-Cas enzymes with nomenclature that may be old and/or new. The skilled person will be able to identify the specific CRISPR-Cas enzyme being referenced in this Application based on the nomenclature that is used, whether it is old (i.e., “legacy”) or new nomenclature. CRISPR-Cas nomenclature is extensively discussed in Makarova et al., “Classification and Nomenclature of CRISPR-Cas Systems: Where from Here?,” The CRISPR Journal, Vol. 1. No. 5, 2018, the entire contents of which are incorporated herein by reference. The particular CRISPR-Cas nomenclature used in any given instance in this Application is not limiting in any way and the skilled person will be able to identify which CRISPR-Cas enzyme is being referenced.

For example, the following type II, type V, and type VI Class 2 CRISPR-Cas enzymes have the following art-recognized old (i.e., legacy) and new names. Each of these enzymes, and/or variants thereof, may be used with the prime editors utilized in the methods and compositions described herein:

| | |
|---|---|
| Legacy nomenclature | Current nomenclature* |

type II CRISPR-Cas enzymes

| | |
|---|---|
| Cas9 | same |

type V CRISPR-Cas enzymes

| | |
|---|---|
| Cpf1 | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |

type VI CRISPR-Cas enzymes

| | |
|---|---|
| C2c2 | Cas13a |
| Cas13d | same |
| C2c7 | Cas13c |
| C2c6 | Cas13b |

*See Makarova et al., The CRISPR Journal, Vol. 1, No. 5, 2018

Without being bound by theory, the mechanism of action of certain napDNAbp contemplated herein includes the step of forming an R-loop whereby the napDNAbp induces the unwinding of a double-strand DNA target, thereby separating the strands in the region bound by the napDNAbp. The guide RNA spacer then hybridizes to the “target strand” at a region that is complementary to the protospacer sequence. This displaces a “non-target strand” that is complementary to the target strand, which forms the single strand region of the R-loop. In some embodiments, the napDNAbp includes one or more nuclease activities, which then cut the DNA leaving various types of lesions. For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”).

The below description of various napDNAbps which can be used in connection with the prime editors utilized in the presently disclosed methods and compositions is not meant to be limiting in any way. The prime editors may comprise the canonical SpCas9, or any ortholog Cas9 protein, or any variant Cas9 protein—including any naturally occurring variant, mutant, or otherwise engineered version of Cas9—that is known or that can be made or evolved through a directed evolutionary or otherwise mutagenic process. In various embodiments, the Cas9 or Cas9 variants have a nickase activity, i.e., only cleave one strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats).

The prime editors utilized in the methods and compositions described herein may also comprise Cas9 equivalents, including Cas12a (Cpf1) and Cas12b1 proteins which are the result of convergent evolution. The napDNAbps used herein (e.g., SpCas9, Cas9 variant, or Cas9 equivalents) may also contain various modifications that alter/enhance their PAM specificities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a reference SpCas9 canonical sequence or a reference Cas9 equivalent (e.g., Cas12a (Cpf1)).

The napDNAbp can be a CRISPR (clustered regularly interspaced short palindromic repeat)-associated nuclease. As outlined above, CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (mc), and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a napDNAbp that is mutated to with respect to a corresponding wild-type enzyme such that the mutated napDNAbp lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, or to equivalent amino acid positions in other Cas9 variants or Cas9 equivalents.

As used herein, the term “Cas protein” refers to a full-length Cas protein obtained from nature, a recombinant Cas protein having a sequences that differs from a naturally occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas 9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any Class 2 CRISPR system (e.g., type II, V, VI), including Cas12a (Cpf1), Cas12e (CasX), Cas12b1 (C2c1), Cas12b2, Cas12c (C2c3), C2c4, C2c8, C2c5, C2c10, C2c9 Cas13a (C2c2), Cas13d, Cas13c (C2c7), Cas13b (C2c6), and Cas13b. Further Cas-equivalents are described in Makarova et al., “C2c2 is a single-component programmable RNA-guided RNA-targeting CRISPR effector,” Science 2016; 353(6299) and Makarova et al., “Classification and Nomenclature of CRISPR-Cas Systems: Where from Here?,” The CRISPR Journal, Vol. 1. No. 5, 2018, the contents of which are incorporated herein by reference.

The terms “Cas9” or “Cas9 nuclease” or “Cas9 moiety” or “Cas9 domain” embrace any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular Cas9 that is employed in the prime editors utilized in the methods and compositions described herein.

As noted herein, Cas9 nuclease sequences and structures are well-known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference).

Examples of Cas9 and Cas9 equivalents are provided as follows; however, these specific examples are not meant to be limiting. The prime editors utilized in the methods and compositions of the present disclosure may use any suitable napDNAbp, including any suitable Cas9 or Cas9 equivalent.

### A. Wild Type Canonical SaCas9

In one embodiment, the prime editor constructs utilized in the methods and compositions described herein may comprise the “canonical SpCas9” nuclease from S. pyogenes, which has been widely used as a tool for genome engineering and is categorized as the type II subgroup of enzymes of the Class 2 CRISPR-Cas systems. This Cas9 protein is a large, multi-domain protein containing two distinct nuclease domains. Point mutations can be introduced into Cas9 to abolish one or both nuclease activities, resulting in a nickase Cas9 (nCas9) or dead Cas9 (dCas9), respectively, that still retains its ability to bind DNA in a sgRNA-programmed manner. In principle, when fused to another protein or domain, Cas9, or variant thereof (e.g., nCas9) can target that protein to virtually any DNA sequence simply by co-expression with an appropriate sgRNA. As used herein, the canonical SpCas9 protein refers to the wild type protein from Streptococcus pyogenes having the following amino acid sequence:

| | | SEQ |
|---|---|---|
| Description | Sequence | ID NO: |
| SpCas9 | MDKKYSIGLDIGTNSVGWAVITDEYKVPSKKFKVLGNTDRHSIKKNLIGALLED | 2 |
| Streptococcus | SGETAEATRLKRTARRRYTRRKNRICYLQEIFSNEMAKVDDSFFHRLEESELVEE | |
| pyogenes M.1 | DKKHERHPIFGNIVDEVAYHEKYPTIYHLRKKLVDSTDKADLRLIYLALAHMIK | |
| SwissProt | FRGHFLIEGDLNPDNSDVDKLFIQLVQTYNQLFEENPINASGVDAKAILSARLSK | |
| Accession No. | SRRLENLIAQLPGEKKNGLFGNLIALSLGLTPNFKSNFDLAEDAKLQLSKDTYDD | |
| Q99ZW2 | DLDNLLAQIGDQYADLFLAAKNLSDAILLSDILRVNTEITKAPLSASMIKRYDEH | |
| Wild type | HQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDN | |
| | REKIEKILTFRIPYYVGPLARGNSRFAWMTRKSEETITPWNFEEVVDKGASAQSF | |
| | IERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVMKQLKR | |
| | RRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDN | |
| | VPSEEVVKKMKNYWRQLLNAKLITQRKFDNLTKAERGGLSELDKAGFIKRQLV | |
| | ETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEI | |
| | GKATAKYFFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKYGGFDSPT | |
| | VAYSVLVVAKVEKGKSKKLKSVKELLGITIMERSSFEKNPIDFLEAKGYKEVKK | |
| | DLIIKLPKYSLFELENGRKRMLASAGELQKGNELALPSKYVNFLYLASHYEKLK | |
| | GSPEDNEQKQLFVEQHKHYLDEIIEQISEFSKRVILADANLDKVLSAYNKHRDKP | |
| | IREQAENIIHLFTLTNLGAPAAFKYFDTTIDRKRYTSTKEVLDATLIHQSITGLYET | |
| | RIDLSQLGGD | |
| SpCas9 | ATGGATAAAAAATATAGCATTGGCCTGGATATTGGCACCAACAGCGTGGGC | 3 |
| Reverse | TGGGCGGTGATTACCGATGAATATAAAGTGCCGAGCAAAAAATTTAAAGTG | |
| translation of | CTGGGCAACACCGATCGCCATAGCATTAAAAAAAACCTGATTGGCGCGCTG | |
| SwissProt | CTGTTTGATAGCGGCGAAACCGCGGAAGCGACCCGCCTGAAACGCACCGCG | |
| Accession No. | CGCCGCCGCTATACCCGCCGCAAAAACCGCATTTGCTATCTGCAGGAAATTT | |
| Q99ZW2 | TTAGCAACGAAATGGCGAAAGTGGATGATAGCTTTTTTCATCGCCTGGAAGA | |
| Streptococcus | AAGCTTTCTGGTGGAAGAAGATAAAAAACATGAACGCCATCCGATTTTTGG | |
| pyogenes | CAACATTGTGGATGAAGTGGCGTATCATGAAAAATATCCGACCATTTATCAT | |
| | CTGCGCAAAAAACTGGTGGATAGCACCGATAAAGCGGATCTGCGCCTGATT | |
| | TATCTGGCGCTGGCGCATATGATTAAATTTCGCGGCCATTTTCTGATTGAAG | |
| | GCGATCTGAACCCGGATAACAGCGATGTGGATAAACTGTTTATTCAGCTGGT | |
| | GCAGACCTATAACCAGCTGTTTGAAGAAAACCCGATTAACGCGAGCGGCGT | |
| | GGATGCGAAAGCGATTCTGAGCGCGCGCCTGAGCAAAAGCCGCCGCCTGGA | |
| | AAACCTGATTGCGCAGCTGCCGGGCGAAAAAAAAAACGGCCTGTTTGGCAA | |
| | CCTGATTGCGCTGAGCCTGGGCCTGACCCCGAACTTTAAAAGCAACTTTGAT | |
| | CTGGCGGAAGATGCGAAACTGCAGCTGAGCAAAGATACCTATGATGATGAT | |
| | CTGGATAACCTGCTGGCGCAGATTGGCGATCAGTATGCGGATCTGTTTCTGG | |
| | CGGCGAAAAACCTGAGCGATGCGATTCTGCTGAGCGATATTCTGCGCGTGA | |
| | ACACCGAAATTACCAAAGCGCCGCTGAGCGCGAGCATGATTAAACGCTATG | |
| | ATGAACATCATCAGGATCTGACCCTGCTGAAAGCGCTGGTGCGCCAGCAGC | |
| | TGCCGGAAAAATATAAAGAAATTTTTTTTGATCAGAGCAAAAACGGCTATG | |
| | CGGGCTATATTGATGGCGGCGCGAGCCAGGAAGAATTTTATAAATTTATTAA | |
| | ACCGATTCTGGAAAAAATGGATGGCACCGAAGAACTGCTGGTGAAACTGAA | |
| | CCGCGAAGATCTGCTGCGCAAACAGCGCACCTTTGATAACGGCAGCATTCC | |
| | GCATCAGATTCATCTGGGCGAACTGCATGCGATTCTGCGCCGCCAGGAAGAT | |
| | TTTTATCCGTTTCTGAAAGATAACCGCGAAAAAATTGAAAAAATTCTGACCT | |
| | TTCGCATTCCGTATTATGTGGGCCCGCTGGCGCGCGGCAACAGCCGCTTTGC | |
| | GTGGATGACCCGCAAAAGCGAAGAAACCATTACCCCGTGGAACTTTGAAGA | |
| | AGTGGTGGATAAAGGCGCGAGCGCGCAGAGCTTTATTGAACGCATGACCAA | |
| | CTTTGATAAAAACCTGCCGAACGAAAAAGTGCTGCCGAAACATAGCCTGCT | |
| | GTATGAATATTTTACCGTGTATAACGAACTGACCAAAGTGAAATATGTGACC | |
| | GAAGGCATGCGCAAACCGGCGTTTCTGAGCGGCGAACAGAAAAAAGCGATT | |
| | GTGGATCTGCTGTTTAAAACCAACCGCAAAGTGACCGTGAAACAGCTGAAA | |
| | GAAGATTATTTTAAAAAAATTGAATGCTTTGATAGCGTGGAAATTAGCGGCG | |
| | TGGAAGATCGCTTTAACGCGAGCCTGGGCACCTATCATGATCTGCTGAAAAT | |
| | TATTAAAGATAAAGATTTTCTGGATAACGAAGAAAACGAAGATATTCTGGA | |
| | AGATATTGTGCTGACCCTGACCCTGTTTGAAGATCGCGAAATGATTGAAGAA | |
| | CGCCTGAAAACCTATGCGCATCTGTTTGATGATAAAGTGATGAAACAGCTGA | |
| | AACGCCGCCGCTATACCGGCTGGGGCCGCCTGAGCCGCAAACTGATTAACG | |
| | GCATTCGCGATAAACAGAGCGGCAAAACCATTCTGGATTTTCTGAAAAGCG | |
| | ATGGCTTTGCGAACCGCAACTTTATGCAGCTGATTCATGATGATAGCCTGAC | |
| | CTTTAAAGAAGATATTCAGAAAGCGCAGGTGAGCGGCCAGGGCGATAGCCT | |
| | GCATGAACATATTGCGAACCTGGCGGGCAGCCCGGCGATTAAAAAAGGCAT | |
| | TCTGCAGACCGTGAAAGTGGTGGATGAACTGGTGAAAGTGATGGGCCGCCA | |
| | TAAACCGGAAAACATTGTGATTGAAATGGCGCGCGAAAACCAGACCACCCA | |
| | GAAAGGCCAGAAAAACAGCCGCGAACGCATGAAACGCATTGAAGAAGGCA | |
| | TTAAAGAACTGGGCAGCCAGATTCTGAAAGAACATCCGGTGGAAAACACCC | |
| | AGCTGCAGAACGAAAAACTGTATCTGTATTATCTGCAGAACGGCCGCGATA | |
| | TGTATGTGGATCAGGAACTGGATATTAACCGCCTGAGCGATTATGATGTGGA | |
| | TCATATTGTGCCGCAGAGCTTTCTGAAAGATGATAGCATTGATAACAAAGTG | |
| | CTGACCCGCAGCGATAAAAACCGCGGCAAAAGCGATAACGTGCCGAGCGAA | |
| | GAAGTGGTGAAAAAAATGAAAAACTATTGGCGCCAGCTGCTGAACGCGAAA | |
| | CTGATTACCCAGCGCAAATTTGATAACCTGACCAAAGCGGAACGCGGCGGC | |
| | CTGAGCGAACTGGATAAAGCGGGCTTTATTAAACGCCAGCTGGTGGAAACC | |
| | CGCCAGATTACCAAACATGTGGCGCAGATTCTGGATAGCCGCATGAACACC | |
| | AAATATGATGAAAACGATAAACTGATTCGCGAAGTGAAAGTGATTACCCTG | |
| | AAAAGCAAACTGGTGAGCGATTTTCGCAAAGATTTTCAGTTTTATAAAGTGC | |
| | GCGAAATTAACAACTATCATCATGCGCATGATGCGTATCTGAACGCGGTGGT | |
| | GGGCACCGCGCTGATTAAAAAATATCCGAAACTGGAAAGCGAATTTGTGTA | |
| | TGGCGATTATAAAGTGTATGATGTGCGCAAAATGATTGCGAAAAGCGAACA | |
| | GGAAATTGGCAAAGCGACCGCGAAATATTTTTTTTATAGCAACATTATGAAC | |
| | TTTTTTAAAACCGAAATTACCCTGGCGAACGGCGAAATTCGCAAACGCCCGC | |
| | TGATTGAAACCAACGGCGAAACCGGCGAAATTGTGTGGGATAAAGGCCGCG | |
| | ATTTTGCGACCGTGCGCAAAGTGCTGAGCATGCCGCAGGTGAACATTGTGA | |
| | AAAAAACCGAAGTGCAGACCGGCGGCTTTAGCAAAGAAAGCATTCTGCCGA | |
| | AACGCAACAGCGATAAACTGATTGCGCGCAAAAAAGATTGGGATCCGAAAA | |
| | AATATGGCGGCTTTGATAGCCCGACCGTGGCGTATAGCGTGCTGGTGGTGGC | |
| | GAAAGTGGAAAAAGGCAAAAGCAAAAAACTGAAAAGCGTGAAAGAACTGC | |
| | TGGGCATTACCATTATGGAACGCAGCAGCTTTGAAAAAAACCCGATTGATTT | |
| | TCTGGAAGCGAAAGGCTATAAAGAAGTGAAAAAAGATCTGATTATTAAACT | |
| | GCCGAAATATAGCCTGTTTGAACTGGAAAACGGCCGCAAACGCATGCTGGC | |
| | GAGCGCGGGCGAACTGCAGAAAGGCAACGAACTGGCGCTGCCGAGCAAAT | |
| | ATGTGAACTTTCTGTATCTGGCGAGCCATTATGAAAAACTGAAAGGCAGCCC | |
| | GGAAGATAACGAACAGAAACAGCTGTTTGTGGAACAGCATAAACATTATCT | |
| | GGATGAAATTATTGAACAGATTAGCGAATTTAGCAAACGCGTGATTCTGGC | |
| | GGATGCGAACCTGGATAAAGTGCTGAGCGCGTATAACAAACATCGCGATAA | |
| | ACCGATTCGCGAACAGGCGGAAAACATTATTCATCTGTTTACCCTGACCAAC | |
| | CTGGGCGCGCCGGCGGCGTTTAAATATTTTGATACCACCATTGATCGCAAAC | |
| | GCTATACCAGCACCAAAGAAGTGCTGGATGCGACCCTGATTCATCAGAGCA | |
| | TTACCGGCCTGTATGAAACCCGCATTGATCTGAGCCAGCTGGGCGGCGAT | |

The prime editors utilized in the methods and compositions described herein may include canonical SpCas9, or any variant thereof having at least 80%, at least 85%, at least 90%, at least 95%, or at least 99% sequence identity with a wild type Cas9 sequence provided above. These variants may include SpCas9 variants containing one or more mutations, including any known mutation reported with the SwissProt Accession No. Q99ZW2 (SEQ ID NO: 2) entry, which include:

| SpCas9 mutation | Function/Characteristic |
|---|---|
| (relative to the amino acid | (as reported) (see UniProtKB- |
| sequence of the canonical | Q99ZW2 (CAS9_STRPT1) |
| SpCas9 sequence, SEQ | entry-incorporated herein by |
| ID NO: 2) | reference) |
| D10A | Nickase mutant which cleaves |
| | the protospacer strand (but no |
| | cleavage of non-protospacer strand) |
| S15A | Decreased DNA cleavage activity |
| R66A | Decreased DNA cleavage activity |
| R70A | No DNA cleavage |
| R74A | Decreased DNA cleavage |
| R78A | Decreased DNA cleavage |
| 97-150 deletion | No nuclease activity |
| R165A | Decreased DNA cleavage |
| 175-307 deletion | About 50% decreased DNA cleavage |
| 312-409 deletion | No nuclease activity |
| E762A | Nickase |
| H840A | Nickase mutant which cleaves |
| | the non-protospacer strand but |
| | does not cleave the protospacer strand |
| N854A | Nickase |
| N863A | Nickase |
| H982A | Decreased DNA cleavage |
| D986A | Nickase |
| 1099-1368 deletion | No nuclease activity |
| R1333A | Reduced DNA binding |

Other wild type SpCas9 sequences that may be used in the present disclosure, include:

| | | SEQ ID |
|---|---|---|
| Description | Sequence | NO: |
| SpCas9 | ATGGATAAGAAATACTCAATAGGCTTAGATATCGGCACAAATAGCGTCGGAT | 4 |
| Streptococcus | GGGCGGTGATCACTGATGATTATAAGGTTCCGTCTAAAAAGTTCAAGGTTCTG | |
| pyogenes | GGAAATACAGACCGCCACAGTATCAAAAAAAATCTTATAGGGGCTCTTTTATT | |
| MGAS1882 | TGGCAGTGGAGAGACAGCGGAAGCGACTCGTCTCAAACGGACAGCTCGTAGA | |
| wild type | AGGTATACACGTCGGAAGAATCGTATTTGTTATCTACAGGAGATTTTTTCAAA | |
| NC_017053.1 | TGAGATGGCGAAAGTAGATGATAGTTTCTTTCATCGACTTGAAGAGTCTTTTTT | |
| | GGTGGAAGAAGACAAGAAGCATGAACGTCATCCTATTTTTGGAAATATAGTA | |
| | GATGAAGTTGCTTATCATGAGAAATATCCAACTATCTATCATCTGCGAAAAAA | |
| | ATTGGCAGATTCTACTGATAAAGCGGATTTGCGCTTAATCTATTTGGCCTTAGC | |
| | GCATATGATTAAGTTTCGTGGTCATTTTTTGATTGAGGGAGATTTAAATCCTGA | |
| | TAATAGTGATGTGGACAAACTATTTATCCAGTTGGTACAAATCTACAATCAAT | |
| | TATTTGAAGAAAACCCTATTAACGCAAGTAGAGTAGATGCTAAAGCGATTCTT | |
| | TCTGCACGATTGAGTAAATCAAGACGATTAGAAAATCTCATTGCTCAGCTCCC | |
| | CGGTGAGAAGAGAAATGGCTTGTTTGGGAATCTCATTGCTTTGTCATTGGGAT | |
| | TGACCCCTAATTTTAAATCAAATTTTGATTTOGCAGAAGATGCTAAATTACAG | |
| | CTTTCAAAAGATACTTACGATGATGATTTAGATAATTTATTGGCGCAAATTGG | |
| | AGATCAATATGCTGATTTGTTTTTGGCAGCTAAGAATTTATCAGATGCTATTTT | |
| | ACTTTCAGATATCCTAAGAGTAAATAGTGAAATAACTAAGGCTCCCCTATCAG | |
| | CTTCAATGATTAAGCGCTACGATGAACATCATCAAGACTTGACTCTTTTAAAA | |
| | GCTTTAGTTCGACAACAACTTCCAGAAAAGTATAAAGAAATCTTTTTTGATCA | |
| | ATCAAAAAACGGATATGCAGGTTATATTGATGGGGGAGCTAGCCAAGAAGAA | |
| | TTTTATAAATTTATCAAACCAATTTTAGAAAAAATGGATGGTACTGAGGAATT | |
| | ATTGGTGAAACTAAATCGTGAAGATTTGCTGCGCAAGCAACGGACCTTTGACA | |
| | ACGGCTCTATTCCCCATCAAATTCACTTGGGTGAGCTGCATGCTATTTTGAGAA | |
| | GACAAGAAGACTTTTATCCATTTTTAAAAGACAATCGTGAGAAGATTGAAAAA | |
| | ATCTTGACTTTTCGAATTCCTTATTATGTTGGTCCATTGGCGCGTGGCAATAGT | |
| | CGTTTTGCATGGATGACTCGGAAGTCTGAAGAAACAATTACCCCATGGAATTT | |
| | TGAAGAAGTTGTCGATAAAGGTGCTTCAGCTCAATCATTTATTGAACGCATGA | |
| | CAAACTTTGATAAAAATCTTCCAAATGAAAAAGTACTACCAAAACATAGTTTG | |
| | CTTTATGAGTATTTTACGGTTTATAACGAATTGACAAAGGTCAAATATGTTACT | |
| | GAGGGAATGCGAAAACCAGCATTTCTTTCAGGTGAACAGAAGAAAGCCATTG | |
| | TTGATTTACTCTTCAAAACAAATCGAAAAGTAACCGTTAAGCAATTAAAAGAA | |
| | GATTATTTCAAAAAAATAGAATGTTTTGATAGTGTTGAAATTTCAGGAGTTGA | |
| | AGATAGATTTAATGCTTCATTAGGCGCCTACCATGATTTGCTAAAAATTATTA | |
| | AAGATAAAGATTTTTTGGATAATGAAGAAAATGAAGATATCTTAGAGGATATT | |
| | GTTTTAACATTGACCTTATTTGAAGATAGGGGGATGATTGAGGAAAGACTTAA | |
| | AACATATGCTCACCTCTTTGATGATAAGGTGATGAAACAGCTTAAACGTCGCC | |
| | GTTATACTGGTTGGGGACGTTTGTCTCGAAAATTGATTAATGGTATTAGGGAT | |
| | AAGCAATCTGGCAAAACAATATTAGATTTTTTGAAATCAGATGGTTTTGCCAA | |
| | TCGCAATTTTATGCAGCTGATCCATGATGATAGTTTGACATTTAAAGAAGATA | |
| | TTCAAAAAGCACAGGTGTCTGGACAAGGCCATAGTTTACATGAACAGATTGCT | |
| | AACTTAGCTGGCAGTCCTGCTATTAAAAAAGGTATTTTACAGACTGTAAAAAT | |
| | TGTTGATGAACTGGTCAAAGTAATGGGGCATAAGCCAGAAAATATCGTTATTG | |
| | AAATGGCACGTGAAAATCAGACAACTCAAAAGGGCCAGAAAAATTCGCGAGA | |
| | GCGTATGAAACGAATCGAAGAAGGTATCAAAGAATTAGGAAGTCAGATTCTT | |
| | AAAGAGCATCCTGTTGAAAATACTCAATTGCAAAATGAAAAGCTCTATCTCTA | |
| | TTATCTACAAAATGGAAGAGACATGTATGTGGACCAAGAATTAGATATTAATC | |
| | GTTTAAGTGATTATGATGTCGATCACATTGTTCCACAAAGTTTCATTAAAGAC | |
| | GATTCAATAGACAATAAGGTACTAACGCGTTCTGATAAAAATCGTGGTAAATC | |
| | GGATAACGTTCCAAGTGAAGAAGTAGTCAAAAAGATGAAAAACTATTGGAGA | |
| | CAACTTCTAAACGCCAAGTTAATCACTCAACGTAAGTTTGATAATTTAACGAA | |
| | AGCTGAACGTGGAGGTTTGAGTGAACTTGATAAAGCTGGTTTTATCAAACGCC | |
| | AATTGGTTGAAACTCGCCAAATCACTAAGCATGTGGCACAAATTTTGGATAGT | |
| | CGCATGAATACTAAATACGATGAAAATGATAAACTTATTCGAGAGGTTAAAGT | |
| | GATTACCTTAAAATCTAAATTAGTTTCTGACTTCCGAAAAGATTTCCAATTCTA | |
| | TAAAGTACGTGAGATTAACAATTACCATCATGCCCATGATGCGTATCTAAATG | |
| | CCGTCGTTGGAACTGCTTTGATTAAGAAATATCCAAAACTTGAATCGGAGTTT | |
| | GTCTATGGTGATTATAAAGTTTATGATGTTCGTAAAATGATTGCTAAGTCTGA | |
| | GCAAGAAATAGGCAAAGCAACCGCAAAATATTTCTTTTACTCTAATATCATGA | |
| | ACTTCTTCAAAACAGAAATTACACTTGCAAATGGAGAGATTCGCAAACGCCCT | |
| | CTAATCGAAACTAATGGGGAAACTGGAGAAATTGTCTGGGATAAAGGGCGAG | |
| | ATTTTGCCACAGTGCGCAAAGTATTGTCCATGCCCCAAGTCAATATTGTCAAG | |
| | AAAACAGAAGTACAGACAGGCGGATTCTCCAAGGAGTCAATTTTACCAAAAA | |
| | GAAATTCGGACAAGCTTATTGCTCGTAAAAAAGACTGGGATCCAAAAAAATA | |
| | TGGTGGTTTTGATAGTCCAACGGTAGCTTATTCAGTCCTAGTGGTTGCTAAGGT | |
| | GGAAAAAGGGAAATCGAAGAAGTTAAAATCCGTTAAAGAGTTACTAGGGATC | |
| | ACAATTATGGAAAGAAGTTCCTTTGAAAAAAATCCGATTGACTTTTTAGAAGC | |
| | TAAAGGATATAAGGAAGTTAAAAAAGACTTAATCATTAAACTACCTAAATAT | |
| | AGTCTTTTTGAGTTAGAAAACGGTCGTAAACGGATGCTGGCTAGTGCCGGAGA | |
| | ATTACAAAAAGGAAATGAGCTGGCTCTGCCAAGCAAATATGTGAATTTTTTAT | |
| | ATTTAGCTAGTCATTATGAAAAGTTGAAGGGTAGTCCAGAAGATAACGAACA | |
| | AAAACAATTGTTTGTGGAGCAGCATAAGCATTATTTAGATGAGATTATTGAGC | |
| | AAATCAGTGAATTTTCTAAGCGTGTTATTTTAGCAGATGCCAATTTAGATAAA | |
| | GTTCTTAGTGCATATAACAAACATAGAGACAAACCAATACGTGAACAAGCAG | |
| | AAAATATTATTCATTTATTTACGTTGACGAATCTTGGAGCTCCCGCTGCTTTTA | |
| | AATATTTTGATACAACAATTGATCGTAAACGATATACGTCTACAAAAGAAGTT | |
| | TTAGATGCCACTCTTATCCATCAATCCATCACTGGTCTTTATGAAACACGCATT | |
| | GATTTGAGTCAGCTAGGAGOTGACTGA | |
| SpCas9 | MDKKYSIGLDIGTNSVGWAVITDDYKVPSKKFKVLGNTDRHSIKKNLIGALLFGS | 5 |
| Streptococcus | GETAEATRLKRTARRRYTRRKNRICYLQEIFSNEMAKVDDSFFHRLEESFLVEEDK | |
| pyogenes | KHERHPIFGNIVDEVAYHEKYPTIYHLRKKLADSTDKADLRLIYLALAHMIKFRGH | |
| MGAS1882 | FLIEGDLNPDNSDVDKLFIQLVQIYNQLFEENPINASRVDAKAILSARLSKSRRLEN | |
| wild type | LIAQLPGEKRNGLFGNLIALSLGLTPNFKSNFDLAEDAKLQLSKDTYDDDLDNLLA | |
| NC_017053.1 | QIGDQYADLFLAAKNLSDAILLSDILRVNSEITKAPLSASMIKRYDEHHQDLTLLK | |
| | ALVRQQLPEKYKEIFFDQSKNGYAGYIDGGASQEEFYKFIKPILEKMDGTEELLVK | |
| | LNREDLLRKQRTFDNGSIPHQIHLGELHAILRRQEDFYPFLKDNREKIEKILTFRIPY | |
| | YVGPLARGNSRFAWMTRKSEETITPWNFEEVVDKGASAQSFIERMTNEDKNLPNE | |
| | KVLPKHSLLYEYFTVYNELTKVKYVTEGMRKPAFLSGEQKKAIVDLLFKTNRKV | |
| | TVKQLKEDYFKKIECFDSVEISGVEDRFNASLGAYHDLLKIIKDKDFLDNEENEDIL | |
| | EDIVLTLTLFEDRGMIEERLKTYAHLEDDKVMKQLKRRRYTGWGRLSRKLINGIR | |
| | DKQSGKTILDFLKSDGFANRNFMQLIHDDSLTFKEDIQKAQVSGQGHSLHEQIANL | |
| | AGSPAIKKGILQTVKIVDELVKVMGHKPENIVIEMARENQTTQKGQKNSRERMKR | |
| | IEEGIKELGSQILKEHPVENTQLQNEKLYLYYLQNGRDMYVDQELDINRLSDYDV | |
| | DHIVPQSFIKDDSIDNKVLTRSDKNRGKSDNVPSEEVVKKMKNYWRQLLNAKLIT | |
| | QRKFDNLTKAERGGLSELDKAGFIKRQLVETRQITKHVAQILDSRMNTKYDENDK | |
| | LIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRK | |
| | RPLIETNGETGEIVWDKGRDFATVRKVLSMPQVNIVKKTEVQTGGFSKESILPKRN | |
| | SDKLIARKKDWDPKKYGGFDSPTVAYSVLVVAKVEKGKSKKLKSVKELLGITIM | |
| | ERSSFEKNPIDFLEAKGYKEVKKDLIIKLPKYSLFELENGRKRMLASAGELQKGNE | |
| | LALPSKYVNFLYLASHYEKLKGSPEDNEQKQLFVEQHKHYLDEIIEQISEFSKRVIL | |
| | ADANLDKVLSAYNKHRDKPIREQAENIIHLFTLTNLGAPAAFKYEDTTIDRKRYTS | |
| | TKEVLDATLIHQSITGLYETRIDLSQLGGD | |
| SpCas9 | ATGGATAAAAAGTATTCTATTGGTTTAGACATCGGCACTAATTCCGTTGGATG | 6 |
| Streptococcus | GGCTGTCATAACCGATGAATACAAAGTACCTTCAAAGAAATTTAAGGTGTTGG | |
| pyogenes wild | GGAACACAGACCGTCATTCGATTAAAAAGAATCTTATCGGTGCCCTCCTATTC | |
| type | GATAGTGGCGAAACGGCAGAGGCGACTCGCCTGAAACGAACCGCTCGGAGAA | |
| SWBC2D7W014 | GGTATACACGTCGCAAGAACCGAATATGTTACTTACAAGAAATTTTTAGCAAT | |
| | GAGATGGCCAAAGTTGACGATTCTTTCTTTCACCGTTTGGAAGAGTCCTTCCTT | |
| | GTCGAAGAGGACAAGAAACATGAACGGCACCCCATCTTTGGAAACATAGTAG | |
| | ATGAGGTGGCATATCATGAAAAGTACCCAACGATTTATCACCTCAGAAAAAA | |
| | GCTAGTTGACTCAACTGATAAAGCGGACCTGAGGTTAATCTACTTGGCTCTTG | |
| | CCCATATGATAAAGTTCCGTGGGCACTTTCTCATTGAGGGTGATCTAAATCCG | |
| | GACAACTCGGATGTCGACAAACTGTTCATCCAGTTAGTACAAACCTATAATCA | |
| | GTTGTTTGAAGAGAACCCTATAAATGCAAGTGGCGTGGATGCGAAGGCTATTC | |
| | TTAGCGCCCGCCTCTCTAAATCCCGACGGCTAGAAAACCTGATCGCACAATTA | |
| | CCCGGAGAGAAGAAAAATGGGTTGTTCGGTAACCTTATAGCGCTCTCACTAGG | |
| | CCTGACACCAAATTTTAAGTCGAACTTCGACTTAGCTGAAGATGCCAAATTGC | |
| | AGCTTAGTAAGGACACGTACGATGACGATCTCGACAATCTACTGGCACAAATT | |
| | GGAGATCAGTATGCGGACTTATTTTTGGCTGCCAAAAACCTTAGCGATGCAAT | |
| | CCTCCTATCTGACATACTGAGAGTTAATACTGAGATTACCAAGGCGCCGTTAT | |
| | CCGCTTCAATGATCAAAAGGTACGATGAACATCACCAAGACTTGACACTTCTC | |
| | AAGGCCCTAGTCCGTCAGCAACTGCCTGAGAAATATAAGGAAATATTCTTTGA | |
| | TCAGTCGAAAAACGGGTACGCAGGTTATATTGACGGCGGAGCGAGTCAAGAG | |
| | GAATTCTACAAGTTTATCAAACCCATATTAGAGAAGATGGATGGGACGGAAG | |
| | AGTTGCTTGTAAAACTCAATCGCGAAGATCTACTGCGAAAGCAGCGGACTTTC | |
| | GACAACGGTAGCATTCCACATCAAATCCACTTAGGCGAATTGCATGCTATACT | |
| | TAGAAGGCAGGAGGATTTTTATCCGTTCCTCAAAGACAATCGTGAAAAGATTG | |
| | AGAAAATCCTAACCTTTCGCATACCTTACTATGTGGGACCCCTGGCCCGAGGG | |
| | AACTCTCGGTTCGCATGGATGACAAGAAAGTCCGAAGAAACGATTACTCCATG | |
| | GAATTTTGAGGAAGTTGTCGATAAAGGTGCGTCAGCTCAATCGTTCATCGAGA | |
| | GGATGACCAACTTTGACAAGAATTTACCGAACGAAAAAGTATTGCCTAAGCA | |
| | CAGTTTACTTTACGAGTATTTCACAGTGTACAATGAACTCACGAAAGTTAAGT | |
| | ATGTCACTGAGGGCATGCGTAAACCCGCCTTTCTAAGCGGAGAACAGAAGAA | |
| | AGCAATAGTAGATCTGTTATTCAAGACCAACCGCAAAGTGACAGTTAAGCAAT | |
| | TGAAAGAGGACTACTTTAAGAAAATTGAATGCTTCGATTCTGTCGAGATCTCC | |
| | GGGGTAGAAGATCGATTTAATGCGTCACTTGGTACGTATCATGACCTCCTAAA | |
| | GATAATTAAAGATAAGGACTTCCTGGATAACGAAGAGAATGAAGATATCTTA | |
| | GAAGATATAGTGTTGACTCTTACCCTCTTTGAAGATCGGGAAATGATTGAGGA | |
| | AAGACTAAAAACATACGCTCACCTGTTCGACGATAAGGTTATGAAACAGTTAA | |
| | AGAGGCGTCGCTATACGGGCTGGGGACGATTGTCGCGGAAACTTATCAACGG | |
| | GATAAGAGACAAGCAAAGTGGTAAAACTATTCTCGATTTTCTAAAGAGCGAC | |
| | GGCTTCGCCAATAGGAACTTTATGCAGCTGATCCATGATGACTCTTTAACCTTC | |
| | AAAGAGGATATACAAAAGGCACAGGTTTCCGGACAAGGGGACTCATTGCACG | |
| | AACATATTGCGAATCTTGCTGGTTCGCCAGCCATCAAAAAGGGCATACTCCAG | |
| | ACAGTCAAAGTAGTGGATGAGCTAGTTAAGGTCATGGGACGTCACAAACCGG | |
| | AAAACATTGTAATCGAGATGGCACGCGAAAATCAAACGACTCAGAAGGGGCA | |
| | AAAAAACAGTCGAGAGCGGATGAAGAGAATAGAAGAGGGTATTAAAGAACT | |
| | GGGCAGCCAGATCTTAAAGGAGCATCCTGTGGAAAATACCCAATTGCAGAAC | |
| | GAGAAACTTTACCTCTATTACCTACAAAATGGAAGGGACATGTATGTTGATCA | |
| | GGAACTGGACATAAACCGTTTATCTGATTACGACGTCGATCACATTGTACCCC | |
| | AATCCTTTTTGAAGGACGATTCAATCGACAATAAAGTGCTTACACGCTCGGAT | |
| | AAGAACCGAGGGAAAAGTGACAATGTTCCAAGCGAGGAAGTCGTAAAGAAA | |
| | ATGAAGAACTATTGGCGGCAGCTCCTAAATGCGAAACTGATAACGCAAAGAA | |
| | AGTTCGATAACTTAACTAAAGCTGAGAGGGGTGGCTTGTCTGAACTTGACAAG | |
| | GCCGGATTTATTAAACGTCAGCTCGTGGAAACCCGCCAAATCACAAAGCATGT | |
| | TGCACAGATACTAGATTCCCGAATGAATACGAAATACGACGAGAACGATAAG | |
| | CTGATTCGGGAAGTCAAAGTAATCACTTTAAAGTCAAAATTGGTGTCGGACTT | |
| | CAGAAAGGATTTTCAATTCTATAAAGTTAGGGAGATAAATAACTACCACCATG | |
| | CGCACGACGCTTATCTTAATGCCGTCGTAGGGACCGCACTCATTAAGAAATAC | |
| | CCGAAGCTAGAAAGTGAGTTTGTGTATGGTGATTACAAAGTTTATGACGTCCG | |
| | TAAGATGATCGCGAAAAGCGAACAGGAGATAGGCAAGGCTACAGCCAAATAC | |
| | TTCTTTTATTCTAACATTATGAATTTCTTTAAGACGGAAATCACTCTGGCAAAC | |
| | GGAGAGATACGCAAACGACCTTTAATTGAAACCAATGGGGAGACAGGTGAAA | |
| | TCGTATGGGATAAGGGCCGGGACTTCGCGACGGTGAGAAAAGTTTTGTCCATG | |
| | CCCCAAGTCAACATAGTAAAGAAAACTGAGGTGCAGACCGGAGGGTTTTCAA | |
| | AGGAATCGATTCTTCCAAAAAGGAATAGTGATAAGCTCATCGCTCGTAAAAA | |
| | GGACTGGGACCCGAAAAAGTACGGTGGCTTCGATAGCCCTACAGTTGCCTATT | |
| | CTGTCCTAGTAGTGGCAAAAGTTGAGAAGGGAAAATCCAAGAAACTGAAGTC | |
| | AGTCAAAGAATTATTGGGGATAACGATTATGGAGCGCTCGTCTTTTGAAAAGA | |
| | ACCCCATCGACTTCCTTGAGGCGAAAGGTTACAAGGAAGTAAAAAAGGATCT | |
| | CATAATTAAACTACCAAAGTATAGTCTGTTTGAGTTAGAAAATGGCCGAAAAC | |
| | GGATGTTGGCTAGCGCCGGAGAGCTTCAAAAGGGGAACGAACTCGCACTACC | |
| | GTCTAAATACGTGAATTTCCTGTATTTAGCGTCCCATTACGAGAAGTTGAAAG | |
| | GTTCACCTGAAGATAACGAACAGAAGCAACTTTTTGTTGAGCAGCACAAACAT | |
| | TATCTCGACGAAATCATAGAGCAAATTTCGGAATTCAGTAAGAGAGTCATCCT | |
| | AGCTGATGCCAATCTGGACAAAGTATTAAGCGCATACAACAAGCACAGGGAT | |
| | AAACCCATACGTGAGCAGGCGGAAAATATTATCCATTTGTTTACTCTTACCAA | |
| | CCTCGGCGCTCCAGCCGCATTCAAGTATTTTGACACAACGATAGATCGCAAAC | |
| | GATACACTTCTACCAAGGAGGTGCTAGACGCGACACTGATTCACCAATCCATC | |
| | ACGGGATTATATGAAACTCGGATAGATTTGTCACAGCTTGGGGGTGACGGATC | |
| | CCCCAAGAAGAAGAGGAAAGTCTCGAGCGACTACAAAGACCATGACGGTGAT | |
| | TATAAAGATCATGACATCGATTACAAGGATGACGATGACAAGGCTGCAGGA | |
| SpCas9 | MDKKYSIGLDIGTNSVGWAVITDEYKVPSKKFKVLGNTDRHSIKKNLIGALLFDS | 7 |
| Streptococcus | GETAEATRLKRTARRRYTRRKNRICYLQEIFSNEMAKVDDSFFHRLEESFLVEEDK | |
| pyogenes wild | KHERHPIFGNIVDEVAYHEKYPTIYHLRKKLVDSTDKADLRLIYLALAHMIKFRGH | |
| type | FLIEGDLNPDNSDVDKLFIQLVQTYNQLFEENPINASGVDAKAILSARLSKSRRLEN | |
| Encoded product | LIAQLPGEKKNGLFGNLIALSLGLTPNFKSNEDLAEDAKLQLSKDTYDDDLDNLL | |
| of | AQIGDQYADLFLAAKNLSDAILLSDILRVNTEITKAPLSASMIKRYDEHHQDLTLL | |
| SWBC2D7W014 | KALVRQQLPEKYKEIFFDQSKNGYAGYIDGGASQEEFYKFIKPILEKMDGTEELLV | |
| | KLNREDLLRKQRTEDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLP | |
| | NEKVLPKHSLLYEYFTVYNELTKVKYVTEGMRKPAFLSGEQKKAIVDLLFKTNR | |
| | KVTVKQLKEDYFKKIECFDSVEISGVEDRFNASLGTYHDLLKIIKDKDFLDNEENE | |
| | DILEDIVLTLTLFEDREMIEERLKTYAHLEDDKVMKQLKRRRYTGWGRLSRKLIN | |
| | GIRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSR | |
| | ERMKRIEEGIKELGSQILKEHPVENTQLQNEKLYLYYLQNGRDMYVDQELDINRL | |
| | SDYDVDHIVPQSFLKDDSIDNKVLTRSDKNRGKSDNVPSEEVVKKMKNYWRQLL | |
| | NAKLITQRKFDNLTKAERGGLSELDKAGFIKRQLVETRQITKHVAQILDSRMNTK | |
| | YDENDKLIREVKVITLKSKLVSDFRKDFQFYKVREINNYHHAHDAYLNAVVGTA | |
| | LIKKYPKLESEFVYGDYKVYDVRKMIAKSEQEIGKATAKYFFYSNIMNFFKTEITL | |
| | ANGEIRKRPLIETNGETGEIVWDKGRDFATVRKVLSMPQVNIVKKTEVQTGGFSK | |
| | ESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPAAFKYEDTTI | |
| | DRKRYTSTKEVLDATLIHQSITGLYETRIDLSQLGGDGSPKKKRKVSSDYKDHDG | |
| | DYKDHDIDYKDDDDKAAG | |
| SpCas9 | ATGGATAAGAAATACTCAATAGGCTTAGATATCGGCACAAATAGCGTCGGAT | 8 |
| Streptococcus | GGGCGGTGATCACTGATGAATATAAGGTTCCGTCTAAAAAGTTCAAGGTTCTG | |
| pyogenes | GGAAATACAGACCGCCACAGTATCAAAAAAAATCTTATAGGGGCTCTTTTATT | |
| MIGAS wild | TGACAGTGGAGAGACAGCGGAAGCGACTCGTCTCAAACGGACAGCTCGTAGA | |
| type | AGGTATACACGTCGGAAGAATCGTATTTGTTATCTACAGGAGATTTTTTCAAA | |
| NC_002737.2 | TGAGATGGCGAAAGTAGATGATAGTTTCTTTCATCGACTTGAAGAGTCTTTTTT | |
| | GGTGGAAGAAGACAAGAAGCATGAACGTCATCCTATTTTTGGAAATATAGTA | |
| | GATGAAGTTGCTTATCATGAGAAATATCCAACTATCTATCATCTGCGAAAAAA | |
| | ATTGGTAGATTCTACTGATAAAGCGGATTTGCGCTTAATCTATTTGGCCTTAGC | |
| | GCATATGATTAAGTTTCGTGGTCATTTTTTGATTGAGGGAGATTTAAATCCTGA | |
| | TAATAGTGATGTGGACAAACTATTTATCCAGTTGGTACAAACCTACAATCAAT | |
| | TATTTGAAGAAAACCCTATTAACGCAAGTGGAGTAGATGCTAAAGCGATTCTT | |
| | TCTGCACGATTGAGTAAATCAAGACGATTAGAAAATCTCATTGCTCAGCTCCC | |
| | CGGTGAGAAGAAAAATGGCTTATTTGGGAATCTCATTGCTTTGTCATTGGGTT | |
| | TGACCCCTAATTTTAAATCAAATTTTGATTTGGCAGAAGATGCTAAATTACAG | |
| | CTTTCAAAAGATACTTACGATGATGATTTAGATAATTTATTGGCGCAAATTGG | |
| | AGATCAATATGCTGATTTGTTTTTGGCAGCTAAGAATTTATCAGATGCTATTTT | |
| | ACTTTCAGATATCCTAAGAGTAAATACTGAAATAACTAAGGCTCCCCTATCAG | |
| | CTTCAATGATTAAACGCTACGATGAACATCATCAAGACTTGACTCTTTTAAAA | |
| | GCTTTAGTTCGACAACAACTTCCAGAAAAGTATAAAGAAATCTTTTTTGATCA | |
| | ATCAAAAAACGGATATGCAGGTTATATTGATGGGGGAGCTAGCCAAGAAGAA | |
| | TTTTATAAATTTATCAAACCAATTTTAGAAAAAATGGATGGTACTGAGGAATT | |
| | ATTGGTGAAACTAAATCGTGAAGATTTGCTGCGCAAGCAACGGACCTTTGACA | |
| | ACGGCTCTATTCCCCATCAAATTCACTTGGGTGAGCTGCATGCTATTTTGAGAA | |
| | GACAAGAAGACTTTTATCCATTTTTAAAAGACAATCGTGAGAAGATTGAAAAA | |
| | ATCTTGACTTTTCGAATTCCTTATTATGTTGGTCCATTGGCGCGTGGCAATAGT | |
| | CGTTTTGCATGGATGACTCGGAAGTCTGAAGAAACAATTACCCCATGGAATTT | |
| | TGAAGAAGTTGTCGATAAAGGTGCTTCAGCTCAATCATTTATTGAACGCATGA | |
| | CAAACTTTGATAAAAATCTTCCAAATGAAAAAGTACTACCAAAACATAGTTTG | |
| | CTTTATGAGTATTTTACGGTTTATAACGAATTGACAAAGGTCAAATATGTTACT | |
| | GAAGGAATGCGAAAACCAGCATTTCTTTCAGGTGAACAGAAGAAAGCCATTG | |
| | TTGATTTACTCTTCAAAACAAATCGAAAAGTAACCGTTAAGCAATTAAAAGAA | |
| | GATTATTTCAAAAAAATAGAATGTTTTGATAGTGTTGAAATTTCAGGAGTTGA | |
| | AGATAGATTTAATGCTTCATTAGGTACCTACCATGATTTGCTAAAAATTATTAA | |
| | AGATAAAGATTTTTTGGATAATGAAGAAAATGAAGATATCTTAGAGGATATTG | |
| | TTTTAACATTGACCTTATTTGAAGATAGGGAGATGATTGAGGAAAGACTTAAA | |
| | ACATATGCTCACCTCTTTGATGATAAGGTGATGAAACAGCTTAAACGTCGCCG | |
| | TTATACTGGTTGGGGACGTTTGTCTCGAAAATTGATTAATGGTATTAGGGATA | |
| | AGCAATCTGGCAAAACAATATTAGATTTTTTGAAATCAGATGGTTTTGCCAAT | |
| | CGCAATTTTATGCAGCTGATCCATGATGATAGTTTGACATTTAAAGAAGACAT | |
| | TCAAAAAGCACAAGTGTCTGGACAAGGCGATAGTTTACATGAACATATTGCA | |
| | AATTTAGCTGGTAGCCCTGCTATTAAAAAAGGTATTTTACAGACTGTAAAAGT | |
| | TGTTGATGAATTGGTCAAAGTAATGGGGCGGCATAAGCCAGAAAATATCGTTA | |
| | TTGAAATGGCACGTGAAAATCAGACAACTCAAAAGGGCCAGAAAAATTCGCG | |
| | AGAGCGTATGAAACGAATCGAAGAAGGTATCAAAGAATTAGGAAGTCAGATT | |
| | CTTAAAGAGCATCCTGTTGAAAATACTCAATTGCAAAATGAAAAGCTCTATCT | |
| | CTATTATCTCCAAAATGGAAGAGACATGTATGTGGACCAAGAATTAGATATTA | |
| | ATCGTTTAAGTGATTATGATGTCGATCACATTGTTCCACAAAGTTTCCTTAAAG | |
| | ACGATTCAATAGACAATAAGGTCTTAACGCGTTCTGATAAAAATCGTGGTAAA | |
| | TCGGATAACGTTCCAAGTGAAGAAGTAGTCAAAAAGATGAAAAACTATTGGA | |
| | GACAACTTCTAAACGCCAAGTTAATCACTCAACGTAAGTTTGATAATTTAACG | |
| | AAAGCTGAACGTGGAGGTTTGAGTGAACTTGATAAAGCTGGTTTTATCAAACG | |
| | CCAATTGGTTGAAACTCGCCAAATCACTAAGCATGTGGCACAAATTTTGGATA | |
| | GTCGCATGAATACTAAATACGATGAAAATGATAAACTTATTCGAGAGGTTAAA | |
| | GTGATTACCTTAAAATCTAAATTAGTTTCTGACTTCCGAAAAGATTTCCAATTC | |
| | TATAAAGTACGTGAGATTAACAATTACCATCATGCCCATGATGCGTATCTAAA | |
| | TGCCGTCGTTGGAACTGCTTTGATTAAGAAATATCCAAAACTTGAATCGGAGT | |
| | TTGTCTATGGTGATTATAAAGTTTATGATGTTCGTAAAATGATTGCTAAGTCTG | |
| | AGCAAGAAATAGGCAAAGCAACCGCAAAATATTTCTTTTACTCTAATATCATG | |
| | AACTTCTTCAAAACAGAAATTACACTTGCAAATGGAGAGATTCGCAAACGCCC | |
| | TCTAATCGAAACTAATGGGGAAACTGGAGAAATTGTCTGGGATAAAGGGCGA | |
| | GATTTTGCCACAGTGCGCAAAGTATTGTCCATGCCCCAAGTCAATATTGTCAA | |
| | GAAAACAGAAGTACAGACAGGCGGATTCTCCAAGGAGTCAATTTTACCAAAA | |
| | AGAAATTCGGACAAGCTTATTGCTCGTAAAAAAGACTGGGATCCAAAAAAAT | |
| | ATGGTGGTTTTGATAGTCCAACGGTAGCTTATTCAGTCCTAGTGGTTGCTAAG | |
| | GTGGAAAAAGGGAAATCGAAGAAGTTAAAATCCGTTAAAGAGTTACTAGGGA | |
| | TCACAATTATGGAAAGAAGTTCCTTTGAAAAAAATCCGATTGACTTTTTAGAA | |
| | GCTAAAGGATATAAGGAAGTTAAAAAAGACTTAATCATTAAACTACCTAAAT | |
| | ATAGTCTTTTTGAGTTAGAAAACGGTCGTAAACGGATGCTGGCTAGTGCCGGA | |
| | GAATTACAAAAAGGAAATGAGCTGGCTCTGCCAAGCAAATATGTGAATTTTTT | |
| | ATATTTAGCTAGTCATTATGAAAAGTTGAAGGGTAGTCCAGAAGATAACGAAC | |
| | AAAAACAATTGTTTGTGGAGCAGCATAAGCATTATTTAGATGAGATTATTGAG | |
| | CAAATCAGTGAATTTTCTAAGCGTGTTATTTTAGCAGATGCCAATTTAGATAA | |
| | AGTTCTTAGTGCATATAACAAACATAGAGACAAACCAATACGTGAACAAGCA | |
| | GAAAATATTATTCATTTATTTACGTTGACGAATCTTGGAGCTCCCGCTGCTTTT | |
| | AAATATTTTGATACAACAATTGATCGTAAACGATATACGTCTACAAAAGAAGT | |
| | TTTAGATGCCACTCTTATCCATCAATCCATCACTGGTCTTTATGAAACACGCAT | |
| | TGATTTGAGTCAGCTAGGAGGTGACTGA | |
| SpCas9 | MDKKYSIGLDIGTNSVGWAVITDEYKVPSKKFKVLGNTDRHSIKKNLIGALLEDS\| | 2 |
| Streptococcus | GETAEATRLKRTARRRYTRRKNRICYLQEIFSNEMAKVDDSFFHRLEESFLVEEDK | |
| pyogenes | KHERHPIFGNIVDEVAYHEKYPTIYHLRKKLVDSTDKADLRLIYLALAHMIKFRGH | |
| MIGAS wild | FLIEGDLNPDNSDVDKLFIQLVQTYNQLFEENPINASGVDAKAILSARLSKSRRLEN | |
| type | LIAQLPGEKKNGLFGNLIALSLGLTPNFKSNEDLAEDAKLQLSKDTYDDDLDNLL | |
| Encoded product | AQIGDQYADLFLAAKNLSDAILLSDILRVNTEITKAPLSASMIKRYDEHHQDLTLL | |
| of NC_002737.2 | KALVRQQLPEKYKEIFFDQSKNGYAGYIDGGASQEEFYKFIKPILEKMDGTEELLV | |
| (100% identical | KLNREDLLRKQRTEDNGSIPHQIHLGELHAILRRQEDFYPFLKDNREKIEKILTFRIP | |
| to the canonical | YYVGPLARGNSRFAWMTRKSEETITPWNFEEVVDKGASAQSFIERMTNFDKNLP | |
| Q99ZW2 | NEKVLPKHSLLYEYFTVYNELTKVKYVTEGMRKPAFLSGEQKKAIVDLLFKTNR | |
| wild type) | KVTVKQLKEDYFKKIECFDSVEISGVEDRFNASLGTYHDLLKIIKDKDFLDNEENE | |
| | DILEDIVLTLTLFEDREMIEERLKTYAHLEDDKVMKQLKRRRYTGWGRLSRKLIN | |
| | GIRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSR | |
| | ERMKRIEEGIKELGSQILKEHPVENTQLQNEKLYLYYLQNGRDMYVDQELDINRL | |
| | SDYDVDHIVPQSFLKDDSIDNKVLTRSDKNRGKSDNVPSEEVVKKMKNYWRQLL | |
| | NAKLITQRKFDNLTKAERGGLSELDKAGFIKRQLVETRQITKHVAQILDSRMNTK | |
| | YDENDKLIREVKVITLKSKLVSDFRKDFQFYKVREINNYHHAHDAYLNAVVGTA | |
| | LIKKYPKLESEFVYGDYKVYDVRKMIAKSEQEIGKATAKYFFYSNIMNFFKTEITL | |
| | ANGEIRKRPLIETNGETGEIVWDKGRDFATVRKVLSMPQVNIVKKTEVQTGGFSK | |
| | ESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPAAFKYFDTTI | |
| | DRKRYTSTKEVLDATLIHQSITGLYETRIDLSQLGGD | |

The prime editors utilized in the methods and compositions described herein may include any of the above SpCas9 sequences, or any variant thereof having at least 80%, at least 85%, at least 90%, at least 95%, or at least 99% sequence identity thereto.

### B. Wild Type Cas9 Orthologs

In other embodiments, the Cas9 protein can be a wild type Cas9 ortholog from another bacterial species different from the canonical Cas9 from S. pyogenes. For example, the following Cas9 orthologs can be used in connection with the prime editor constructs utilized in the methods and compositions described in this specification. In addition, any variant Cas9 orthologs having at least 80%, at least 85%, at least 90%, at least 95%, or at least 99% sequence identity to any of the below orthologs may also be used with the prime editors.

| Description | Sequence |
|---|---|
| LfCas9 | MKEYHIGLDIGTSSIGWAVTDSQFKLMRIKGKTAIGVRLFEEGKTAAERRTFRTTRRRLKR |
| Lactobacillus | RKWRLHYLDEIFAPHLQEVDENFLRRLKQSNIHPEDPTKNQAFIGKLLFPDLLKKNERGYP |
| fermentum wild | TLIKMRDELPVEQRAHYPVMNIYKLREAMINEDRQFDLREVYLAVHHIVKYRGHFLNNA |
| type | SVDKFKVGRIDFDKSFNVLNEAYEELQNGEGSFTIEPSKVEKIGQLLLDTKMRKLDRQKA |
| GenBank: | VAKLLEVKVADKEETKRNKQIATAMSKLVLGYKADFATVAMANGNEWKIDLSSETSED |
| SNX31424.11 | EIEKFREELSDAQNDILTEITSLFSQIMLNEIVPNGMSISESMMDRYWTHERQLAEVKEYLA |
| | TQPASARKEFDQVYNKYIGQAPKERGFDLEKGLKKILSKKENWKEIDELLKAGDELPKQR |
| | TSANGVIPHQMHQQELDRIIEKQAKYYPWLATENPATGERDRHQAKYELDQLVSFRIPYY |
| | VGPLVTPEVQKATSGAKFAWAKRKEDGEITPWNLWDKIDRAESAEAFIKRMTVKDTYLL |
| | NEDVLPANSLLYQKYNVLNELNNVRVNGRRLSVGIKQDIYTELFKKKKTVKASDVASLV |
| | MAKTRGVNKPSVEGLSDPKKENSNLATYLDLKSIVGDKVDDNRYQTDLENIIEWRSVFED |
| | GEIFADKLTEVEWLTDEQRSALVKKRYKGWGRLSKKLLTGIVDENGQRIIDLMWNTDQN |
| | FKEIVDQPVFKEQIDQLNQKAITNDGMTLRERVESVLDDAYTSPQNKKAIWQVVRVVEDI |
| | VKAVGNAPKSISIEFARNEGNKGEITRSRRTQLQKLFEDQAHELVKDTSLTEELEKAPDLS |
| | DRYYFYFTQGGKDMYTGDPINFDEISTKYDIDHILPQSFVKDNSLDNRVLTSRKENNKKS |
| | DQVPAKLYAAKMKPYWNQLLKQGLITQRKFENLTKDVDQNIKYRSLGFVKRQLVETRQ |
| | VIKLTANILGSMYQEAGTEIIETRAGLTKQLREEFDLPKVREVNDYHHAVDAYLTTFAGQ |
| | YLNRRYPKLRSFFVYGEYMKFKHGSDLKLRNFNFFHELMEGDKSQGKVVDQQTGELITT |
| | RDEVAKSFDRLLNMKYMLVSKEVHDRSDQLYGATIVTAKESGKLTSPIEIKKNRLVDLYG |
| | AYTNGTSAFMTIIKFTGNKPKYKVIGIPTTSAASLKRAGKPGSESYNQELHRIIKSNPKVKK |
| | GFEIVVPHVSYGQLIVDGDCKFTLASPTVQHPATQLVLSKKSLETISSGYKILKDKPAIANE |
| | RLIRVFDEVVGQMNRYFTIFDQRSNRQKVADARDKFLSLPTESKYEGAKKVQVGKTEVIT |
| | NLLMGLHANATQGDLKVLGLATFGFFQSTTGLSLSEDTMIVYQSPTGLFERRICLKDI |
| | (SEQ ID NO: 9) |
| SaCas9 | MDKKYSIGLDIGTNSVGWAVITDEYKVPSKKFKVLGNTDRHSIKKNLIGALLFDSGETAE |
| Staphylococcus | ATRLKRTARRRYTRRKNRICYLQEIFSNEMAKVDDSFFHRLEESFLVEEDKKHERHPIFGN |
| aureus wild type | IVDEVAYHEKYPTIYHLRKKLVDSTDKADLRLIYLALAHMIKFRGHFLIEGDLNPDNSDVD |
| GenBank: | KLFIQLVQTYNQLFEENPINASGVDAKAILSARLSKSRRLENLIAQLPGEKKNGLFGNLIAL |
| AYD60528.1 | SLGLTPNFKSNFDLAEDAKLQLSKDTYDDDLDNLLAQIGDQYADLFLAAKNLSDAILLSDI |
| | LRVNTEITKAPLSASMIKRYDEHHQDLTLLKALVRQQLPEKYKEIFFDQSKNGYAGYIDG |
| | GASQEEFYKFIKPILEKMDGTEELLVKLNREDLLRKQRTFDNGSIPHQIHLGELHAILRRQE |
| | DFYPFLKDNREKIEKILTFRIPYYVGPLARGNSRFAWMTRKSEETITPWNFEEVVDKGASA |
| | QSFIERMTNFDKNLPNEKVLPKHSLLYEYFTVYNELTKVKYVTEGMRKPAFLSGEQKKAI |
| | VDLLFKTNRKVTVKQLKEDYFKKIECFDSVEISGVEDRENASLGTYHDLLKIIKDKDELDN |
| | EENEDILEDIVLTLTLFEDREMIEERLKTYAHLEDDKVMKQLKRRRYTGWGRLSRKLINGI |
| | RDKQSGKTILDFLKSDGFANRNFMQLIHDDSLTFKEDIQKAQVSGQGDSLHEHIANLAGSP |
| | AIKKGILQTVKVVDELVKVMGRHKPENIVIEMARENQTTQKGQKNSRERMKRIEEGIKEL |
| | GSQILKEHPVENTQLQNEKLYLYYLQNGRDMYVDQELDINRLSDYDVDHIVPQSFLKDDS |
| | IDNKVLTRSDKNRGKSDNVPSEEVVKKMKNYWRQLLNAKLITQRKFDNLTKAERGGLSE |
| | LDKAGFIKRQLVETRQITKHVAQILDSRMNTKYDENDKLIREVKVITLKSKLVSDFRKDFQ |
| | FYKVREINNYHHAHDAYLNAVVGTALIKKYPKLESEFVYGDYKVYDVRKMIAKSEQEIG |
| | KATAKYFFYSNIMNFFKTEITLANGEIRKRPLIETNGETGEIVWDKGRDFATVRKVLSMPQ |
| | VNIVKKTEVQTGGFSKESILPKRNSDKLIARKKDWDPKKYGGFDSPTVAYSVLVVAKVEK |
| | GKSKKLKSVKELLGITIMERSSFEKNPIDFLEAKGYKEVKKDLIIKLPKYSLFELENGRKRM |
| | LASAGELQKGNELALPSKYVNFLYLASHYEKLKGSPEDNEQKQLFVEQHKHYLDEIIEQIS |
| | EFSKRVILADANLDKVLSAYNKHRDKPIREQAENIIHLFTLTNLGAPAAFKYFDTTIDRKR |
| | YTSTKEVLDATLIHQSITGLYETRIDLSQLGGD (SEQ ID NO: 2) |
| SaCas9 | MGKRNYILGLDIGITSVGYGIIDYETRDVIDAGVRLFKEANVENNEGRRSKRGARRLKRR |
| Staphylococcus | RRHRIQRVKKLLFDYNLLTDHSELSGINPYEARVKGLSQKLSEEEFSAALLHLAKRRGVH |
| aureus | NVNEVEEDTGNELSTKEQISRNSKALEEKYVAELQLERLKKDGEVRGSINRFKTSDYVKE |
| | AKQLLKVQKAYHQLDQSFIDTYIDLLETRRTYYEGPGEGSPFGWKDIKEWYEMLMGHCT |
| | YFPEELRSVKYAYNADLYNALNDLNNLVITRDENEKLEYYEKFQIIENVFKQKKKPTLKQI |
| | AKEILVNEEDIKGYRVTSTGKPEFTNLKVYHDIKDITARKEHIENAELLDQIAKILTIYQSSE |
| | DIQEELTNLNSELTQEEIEQISNLKGYTGTHNLSLKAINLILDELWHTNDNQIAIFNRLKLVP |
| | KKVDLSQQKEIPTTLVDDFILSPVVKRSFIQSIKVINAIIKKYGLPNDIIIELAREKNSKDAQK |
| | MINEMQKRNRQTNERIEEIIRTTGKENAKYLIEKIKLHDMQEGKCLYSLEAIPLEDLLNNPF |
| | NYEVDHIIPRSVSFDNSFNNKVLVKQEENSKKGNRTPFQYLSSSDSKISYETFKKHILNLAK |
| | GKGRISKTKKEYLLEERDINRFSVQKDFINRNLVDTRYATRGLMNLLRSYFRVNNLDVKV |
| | KSINGGFTSFLRRKWKFKKERNKGYKHHAEDALIIANADFIFKEWKKLDKAKKVMENQM |
| | FEEKQAESMPEIETEQEYKEIFITPHQIKHIKDFKDYKYSHRVDKKPNRKLINDTLYSTRKD |
| | DKGNTLIVNNLNGLYDKDNDKLKKLINKSPEKLLMYHHDPQTYQKLKLIMEQYGDEKNP |
| | LYKYYEETGNYLTKYSKKDNGPVIKKIKYYGNKLNAHLDITDDYPNSRNKVVKLSLKPY |
| | RFDVYLDNGVYKFVTVKNLDVIKKENYYEVNSKCYEEAKKLKKISNQAEFIASFYKNDLI |
| | KINGEL YRVIGVNNDLLNRIEVNMIDITYREYLENMNDKRPPHIIKTIASKTQSIKKYSTDIL |
| | GNLYEVKSKKHPQIIKK |
| | (SEQ ID NO: 10) |
| StCas9 | MLFNKCIIISINLDFSNKEKCMTKPYSIGLDIGTNSVGWAVITDNYKVPSKKMKVLGNTSK |
| Streptococcus | KYIKKNLLGVLLFDSGITAEGRRLKRTARRRYTRRRNRILYLQEIFSTEMATLDDAFFQRL |
| thermophilus | DDSFLVPDDKRDSKYPIFGNLVEEKVYHDEFPTIYHLRKYLADSTKKADLRLVYLALAHM |
| UniProtKB/ | IKYRGHFLIEGEFNSKNNDIQKNFQDFLDTYNAIFESDLSLENSKQLEEIVKDKISKLEKKD |
| Swiss-Prot: | RILKLFPGEKNSGIFSEFLKLIVGNQADFRKCFNLDEKASLHFSKESYDEDLETLLGYIGDD |
| G3ECR1.2 | YSDVFLKAKKLYDAILLSGFLTVTDNETEAPLSSAMIKRYNEHKEDLALLKEYIRNISLKT |
| Wild type | YNEVFKDDTKNGYAGYIDGKTNQEDFYVYLKNLLAEFEGADYFLEKIDREDFLRKQRTF |
| | DNGSIPYQIHLQEMRAILDKQAKFYPFLAKNKERIEKILTFRIPYYVGPLARGNSDFAWSIR |
| | KRNEKITPWNFEDVIDKESSAEAFINRMTSEDLYLPEEKVLPKHSLLYETFNVYNELTKVR |
| | FIAESMRDYQFLDSKQKKDIVRLYFKDKRKVTDKDIIEYLHAIYGYDGIELKGIEKQFNSSL |
| | STYHDLLNIINDKEFLDDSSNEAIIEEIIHTLTIFEDREMIKQRLSKFENIFDKSVLKKLSRRH |
| | YTGWGKLSAKLINGIRDEKSGNTILDYLIDDGISNRNFMQLIHDDALSFKKKIQKAQIIGDE |
| | DKGNIKEVVKSLPGSPAIKKGILQSIKIVDELVKVMGGRKPESIVVEMARENQYTNQGKSN |
| | SQQRLKRLEKSLKELGSKILKENIPAKLSKIDNNALQNDRLYLYYLQNGKDMYTGDDLDI |
| | DRLSNYDIDHIIPQAFLKDNSIDNKVLVSSASNRGKSDDFPSLEVVKKRKTFWYQLLKSKL |
| | ISQRKEDNLTKAERGGLLPEDKAGFIQRQLVETRQITKHVARLLDEKENNKKDENNRAVR |
| | TVKIITLKSTLVSQFRKDFELYKVREINDFHHAHDAYLNAVIASALLKKYPKLEPEFVYGD |
| | YPKYNSFRERKSATEKVYFYSNIMNIFKKSISLADGRVIERPLIEVNEETGESVWNKESDLA |
| | TVRRVLSYPQVNVVKKVEEQNHGLDRGKPKGLFNANLSSKPKPNSNENLVGAKEYLDPK |
| | KYGGYAGISNSFAVLVKGTIEKGAKKKITNVLEFQGISILDRINYRKDKLNFLLEKGYKDIE |
| | LIIELPKYSLFELSDGSRRMLASILSTNNKRGEIHKGNQIFLSQKFVKLLYHAKRISNTINEN |
| | HRKYVENHKKEFEELFYYILEFNENYVGAKKNGKLLNSAFQSWQNHSIDELCSSFIGPTGS |
| | ERKGLFELTSRGSAADFEFLGVKIPRYRDYTPSSLLKDATLIHQSVTGLYETRIDLAKLGEG |
| | (SEQ ID NO: 11) |
| LcCas9 | MKIKNYNLALTPSTSAVGHVEVDDDLNILEPVHHQKAIGVAKFGEGETAEARRLARSARR |
| Lactobacillus | TTKRRANRINHYFNEIMKPEIDKVDPLMEDRIKQAGLSPLDERKEFRTVIFDRPNIASYYHN |
| crispatus | QFPTIWHLQKYLMITDEKADIRLIYWALHSLLKHRGHFENTTPMSQFKPGKLNLKDDMLA |
| NCBI Reference | LDDYNDLEGLSFAVANSPEIEKVIKDRSMHKKEKIAELKKLIVNDVPDKDLAKRNNKIITQ |
| Sequence: | IVNAIMGNSFHLNFIFDMDLDKLTSKAWSFKLDDPELDTKFDAISGSMTDNQIGIFETLQKI |
| WP_1334780441 | YSAISLLDILNGSSNVVDAKNALYDKHKRDLNLYFKFLNTLPDEIAKTLKAGYTLYIGNRK |
| Wild type | KDLLAARKLLKVNVAKNFSQDDFYKLINKELKSIDKQGLQTRESEKVGELVAQNNFLPV |
| | QRSSDNVFIPYQLNAITFNKILENQGKYYDFLVKPNPAKKDRKNAPYELSQLMQFTIPYYV |
| | GPLVTPEEQVKSGIPKTSRFAWMVRKDNGAITPWNFYDKVDIEATADKFIKRSIAKDSYLL |
| | SELVLPKHSLLYEKYEVENELSNVSLDGKKLSGGVKQILFNEVFKKTNKVNTSRILKALA |
| | KHNIPGSKITGLSNPEEFTSSLQTYNAWKKYFPNQIDNFAYQQDLEKMIEWSTVFEDHKIL |
| | AKKLDEIEWLDDDQKKFVANTRLRGWGRLSKRLLTGLKDNYGKSIMQRLETTKANFQQI |
| | VYKPEFREQIDKISQAAAKNQSLEDILANSYTSPSNRKAIRKTMSVVDEYIKLNHGKEPDK |
| | IFLMFQRSEQEKGKQTEARSKQLNRILSQLKADKSANKLFSKQLADEFSNAIKKSKYKLN |
| | DKQYFYFQQLGRDALTGEVIDYDELYKYTVLHIIPRSKLTDDSQNNKVLTKYKIVDGSVA |
| | LKFGNSYSDALGMPIKAFWTELNRLKLIPKGKLLNLTTDFSTLNKYQRDGYIARQLVETQ |
| | QIVKLLATIMQSRFKHTKIIEVRNSQVANIRYQFDYFRIKNLNEYYRGFDAYLAAVVGTYL |
| | YKVYPKARRLFVYGQYLKPKKTNQENQDMHLDSEKKSQGFNFLWNLLYGKQDQIFVNG |
| | TDVIAFNRKDLITKMNTVYNYKSQKISLAIDYHNGAMFKATLFPRNDRDTAKTRKLIPKK |
| | KDYDTDIYGGYTSNVDGYMLLAEIIKRDGNKQYGFYGVPSRLVSELDTLKKTRYTEYEEK |
| | LKEIIKPELGVDLKKIKKIKILKNKVPFNQVIIDKGSKFFITSTSYRWNYRQLILSAESQQTL |
| | MDLVVDPDFSNHKARKDARKNADERLIKVYEEILYQVKNYMPMFVELHRCYEKLVDAQ |
| | KTFKSLKISDKAMVLNQILILLHSNATSPVLEKLGYHTRFTLGKKHNLISENAVLVTQSITG |
| | LKENHVSIKQML (SEQ ID NO: 12) |
| PdCas9 | MTNEKYSIGLDIGTSSIGFAVVNDNNRVIRVKGKNAIGVRLFDEGKAAADRRSFRTTRRSF |
| Pedicoccus | RTTRRRLSRRRWRLKLLREIFDAYITPVDEAFFIRLKESNLSPKDSKKQYSGDILENDRSDK |
| damnosus | DFYEKYPTIYHLRNALMTEHRKFDVREIYLAIHHIMKERGHELNATPANNEKVGRLNLEE |
| NCBI Reference | \|KFEELNDIYQRVFPDESIEFRTDNLEQIKEVLLDNKRSRADRQRTLVSDIYQSSEDKDIEKR |
| Sequence: | NKAVATEILKASLGNKAKLNVITNVEVDKEAAKEWSITEDSESIDDDLAKIEGQMTDDGH |
| WP_0629132731 | EIIEVLRSLYSGITLSAIVPENHTLSQSMVAKYDLHKDHLKLFKKLINGMTDTKKAKNLRA |
| Wild type | AYDGYIDGVKGKVLPQEDFYKQVQVNLDDSAEANEIQTYIDQDIFMPKQRTKANGSIPHQ |
| | LQQQELDQUIENQKAYYPWLAELNPNPDKKRQQLAKYKLDELVTFRVPYYVGPMITAKD |
| | QKNQSGAEFAWMIRKEPGNITPWNFDQKVDRMATANQFIKRMTTTDTYLLGEDVLPAQS |
| | LLYQKFEVLNELNKIRIDHKPISIEQKQQIFNDLFKQFKNVTIKHLQDYLVSQGQYSKRPLI |
| | EGLADEKRFNSSLSTYSDLCGIFGAKLVEENDRQEDLEKIIEWSTIFEDKKIYRAKLNDLT |
| | WLTDDQKEKLATKRYQGWGRLSRKLLVGLKNSEHRNIMDILWITNENFMQIQAEPDFAK |
| | LVTDANKGMLEKTDSQDVINDLYTSPQNKKAIRQILLVVHDIQNAMHGQAPAKIHVEFAR |
| | GEERNPRRSVQRQRQVEAAYEKVSNELVSAKVRQEFKEAINNKRDFKDRLFLYFMQGGI |
| | DIYTGKQLNIDQLSSYQIDHILPQAFVKDDSLTNRVLTNENQVKADSVPIDIFGKKMLSVW |
| | GRMKDQGLISKGKYRNLTMNPENISAHTENGFINRQLVETRQVIKLAVNILADEYGDSTQI |
| | ISVKADLSHQMREDFELLKNRDVNDYHHAFDAYLAAFIGNYLLKRYPKLESYFVYGDFK |
| | KFTQKETKMRRFNFIYDLKHCDQVVNKETGEILWTKDEDIKYIRHLFAYKKILVSHEVRE |
| | KRGALYNQTIYKAKDDKGSGQESKKLIRIKDDKETKIYGGYSGKSLAYMTIVQITKKNKV |
| | SYRVIGIPTLALARLNKLENDSTENNGELYKIIKPQFTHYKVDKKNGEIIETTDDFKIVVSK |
| | VRFQQLIDDAGQFFMLASDTYKNNAQQLVISNNALKAINNTNITDCPRDDLERLDNLRLD |
| | SAFDEIVKKMDKYFSAYDANNFREKIRNSNLIFYQLPVEDQWENNKITELGKRTVLTRILQ |
| | GLHANATTTDMSIFKIKTPFGQLRQRSGISLSENAQLIYQSPTGLFERRVQLNKIK (SEQ ID |
| | NO: 13) |
| FnCas9 | MKKQKFSDYYLGFDIGTNSVGWCVTDLDYNVLRFNKKDMWGSRLFEEAKTAAERRVQ |
| Fusobaterium | RNSRRRLKRRKWRLNLLEEIFSNEILKIDSNFFRRLKESSLWLEDKSSKEKFTLENDDNYK. |
| nucleatum | DYDFYKQYPTIFHLRNELIKNPEKKDIRLVYLAIHSIFKSRGHFLFEGQNLKEIKNFETLYN |
| NCBI Reference | NLIAFLEDNGINKIIDKNNIEKLEKIVCDSKKGLKDKEKEFKEIFNSDKQLVAIFKLSVGSSV |
| Sequence: | SLNDLFDTDEYKKGEVEKEKISFREQIYEDDKPIYYSILGEKIELLDIAKTFYDFMVLNNILA |
| WP_0607989841 | DSQYISEAKVKLYEEHKKDLKNLKYIIRKYNKGNYDKLFKDKNENNYSAYIGLNKEKSK |
| | KEVIEKSRLKIDDLIKNIKGYLPKVEEIEEKDKAIFNKILNKIELKTILPKQRISDNGTLPYQI |
| | HEAELEKILENQSKYYDELNYEENGIITKDKLLMTFKFRIPYYVGPLNSYHKDKGGNSWIV |
| | RKEEGKILPWNFEQKVDIEKSAEEFIKRMTNKCTYLNGEDVIPKDTFLYSEYVILNELNKV |
| | QVNDEFLNEENKRKIIDELFKENKKVSEKKFKEYLLVKQIVDGTIELKGVKDSENSNYISYI |
| | RFKDIFGEKLNLDIYKEISEKSILWKCLYGDDKKIFEKKIKNEYGDILTKDEIKKINTFKENN |
| | WGRLSEKLLTGIEFINLETGECYSSVMDALRRTNYNLMELLSSKFTLQESINNENKEMNEA |
| | SYRDLIEESYVSPSLKRAIFQTLKIYEEIRKITGRVPKKVFIEMARGGDESMKNKKIPARQE |
| | QLKKLYDSCGNDIANFSIDIKEMKNSLISYDNNSLRQKKLYLYYLQFGKCMYTGREIDLD |
| | RLLQNNDTYDIDHIYPRSKVIKDDSFDNLVLVLKNENAEKSNEYPVKKEIQEKMKSFWRF |
| | LKEKNFISDEKYKRLTGKDDFELRGFMARQLVNVRQTTKEVGKILQQIEPEIKIVYSKAEI |
| | ASSFREMFDFIKVRELNDTHHAKDAYLNIVAGNVYNTKFTEKPYRYLQEIKENYDVKKIY |
| | NYDIKNAWDKENSLEIVKKNMEKNTVNITRFIKEKKGQLFDLNPIKKGETSNEIISIKPKVY |
| | NGKDDKLNEKYGYYKSLNPAYFLYVEHKEKNKRIKSFERVNLVDVNNIKDEKSLVKYLI |
| | ENKKLVEPRVIKKVYKRQVILINDYPYSIVTLDSNKLMDFENLKPLFLENKYEKILKNVIKF |
| | LEDNQGKSEENYKFIYLKKKDRYEKNETLESVKDRYNLEFNEMYDKFLEKLDSKDYKNY |
| | MNNKKYQELLDVKEKFIKLNLFDKAFTLKSFLDLFNRKTMADESKVGLTKYLGKIQKISS |
| | NVLSKNELYLLEESVTGLFVKKIKL (SEQ ID NO: 14) |
| EcCas9 | RRKQRIQILQELLGEEVLKTDPGFFHRMKESRYVVEDKRTLDGKQVELPYALFVDKDYTD |
| Enterococcus | KEYYKQFPTINHLIVYLMTTSDTPDIRLVYLALHYYMKNRGNFLHSGDINNVKDINDILEQ |
| cecorum | LDNVLETFLDGWNLKLKSYVEDIKNIYNRDLGRGERKKAFVNTLGAKTKAEKAFCSLISG |
| NCBI Reference | GSTNLAELFDDSSLKEIETPKIEFASSSLEDKIDGIQEALEDRFAVIEAAKRLYDWKTLTDIL |
| Sequence: | GDSSSLAEARVNSYQMHHEQLLELKSLVKEYLDRKVFQEVFVSLNVANNYPAYIGHTKI |
| WP_0473385011 | NGKKKELEVKRTKRNDFYSYVKKQVIEPIKKKVSDEAVLTKLSEIESLIEVDKYLPLQVNS |
| Wild type | DNGVIPYQVKLNELTRIFDNLENRIPVLRENRDKIIKTFKFRIPYYVGSLNGVVKNGKCTN |
| | WMVRKEEGKIYPWNFEDKVDLEASAEQFIRRMTNKCTYLVNEDVLPKYSLLYSKYLVLS |
| | ELNNLRIDGRPLDVKIKQDIYENVFKKNRKVTLKKIKKYLLKEGIITDDDELSGLADDVKS |
| | SLTAYRDFKEKLGHLDLSEAQMENIILNITLFGDDKKLLKKRLAALYPFIDDKSLNRIATLN |
| | YRDWGRLSERFLSGITSVDQETGELRTIIQCMYETQANLMQLLAEPYHFVEAIEKENPKVD |
| | LESISYRIVNDLYVSPAVKRQIWQTLLVIKDIKQVMKHDPERIFIEMAREKQESKKTKSRK |
| | QVLSEVYKKAKEYEHLFEKLNSLTEEQLRSKKIYLYFTQLGKCMYSGEPIDFENLVSANS |
| | NYDIDHIYPQSKTIDDSENNIVLVKKSLNAYKSNHYPIDKNIRDNEKVKTLWNTLVSKGLI |
| | TKEKYERLIRSTPFSDEELAGFIARQLVETRQSTKAVAEILSNWFPESEIVYSKAKNVSNFR |
| | QDFEILKVRELNDCHHAHDAYLNIVVGNAYHTKFTNSPYRFIKNKANQEYNLRKLLQKV |
| | NKIESNGVVAWVGQSENNPGTIATVKKVIRRNTVLISRMVKEVDGQLFDLTLMKKGKGQ |
| | VPIKSSDERLTDISKYGGYNKATGAYFTFVKSKKRGKVVRSFEYVPLHLSKQFENNNELL |
| | KEYIEKDRGLTDVEILIPKVLINSLFRYNGSLVRITGRGDTRLLLVHEQPLYVSNSFVQQLK |
| | SVSSYKLKKSENDNAKLTKTATEKLSNIDELYDGLLRKLDLPIYSYWFSSIKEYLVESRTK |
| | YIKLSIEEKAL VIFEILHLFQSDAQVPNLKILGLSTKPSRIRIQKNLKDTDKMSIIHQSPSGIFE |
| | HEIELTSL (SEQ ID NO: 15) |
| AhCas9 | MQNGELGITVSSEQVGWAVTNPKYELERASRKDLWGVRLFDKAETAEDRRMFRTNRRL |
| Anaerostipes | NQRKKNRIHYLRDIFHEEVNQKDPNFFQQLDESNFCEDDRTVEFNEDTNLYKNQFPTVYH |
| hadrus | LRKYLMETKDKPDIRLVYLAFSKFMKNRGHFLYKGNLGEVMDFENSMKGFCESLEKENI |
| NCBI Reference | DFPTLSDEQVKEVRDILCDHKIAKTVKKKNIITITKVKSKTAKAWIGLFCGCSVPVKVLFQ |
| Sequence: | DIDEEIVTDPEKISFEDASYDDYIANIEKGVGIYYEAIVSAKMLFDWSILNEILGDHQLLSDA |
| WP_0449242781 | MIAEYNKHHDDLKRLQKIIKGTGSRELYQDIFINDVSGNYVCYVGHAKTMSSADQKQFY |
| Wild type | TFLKNRLKNVNGISSEDAEWIDTEIKNGTLLPKQTKRDNSVIPHQLQLREFELILDNMQEM |
| | YPFLKENREKLLKIFNFVIPYYVGPLKGVVRKGESTNWMVPKKDGVIHPWNFDEMVDKE |
| | ASAECFISRMTGNCSYLFNEKVLPKNSLLYETFEVLNELNPLKINGEPISVELKQRIYEQLF |
| | LTGKKVTKKSLTKYLIKNGYDKDIELSGIDNEFHSNLKSHIDFEDYDNLSDEEVEQIILRITV |
| | FEDKQLLKDYLNREFVKLSEDERKQICSLSYKGWGNLSEMLLNGITVTDSNGVEVSVMD |
| | MLWNTNLNLMQILSKKYGYKAEIEHYNKEHEKTIYNREDLMDYLNIPPAQRRKVNQLITI |
| | VKSLKKTYGVPNKIFFKISREHQDDPKRTSSRKEQLKYLYKSLKSEDEKHLMKELDELND |
| | HELSNDKVYLYFLQKGRCIYSGKKLNLSRLRKSNYQNDIDYIYPLSAVNDRSMNNKVLTG |
| | IQENRADKYTYFPVDSEIQKKMKGFWMELVLQGFMTKEKYFRLSRENDFSKSELVSFIER |
| | EISDNQQSGRMIASVLQYYFPESKIVFVKEKLISSFKRDFHLISSYGHNHLQAAKDAYITIV |
| | VGNVYHTKFTMDPAIYFKNHKRKDYDLNRLFLENISRDGQIAWESGPYGSIQTVRKEYAQ |
| | NHIAVTKRVVEVKGGLFKQMPLKKGHGEYPLKTNDPRFGNIAQYGGYTNVTGSYFVLVE |
| | SMEKGKKRISLEYVPVYLHERLEDDPGHKLLKEYLVDHRKLNHPKILLAKVRKNSLLKID |
| | GFYYRLNGRSGNALILTNAVELIMDDWQTKTANKISGYMKRRAIDKKARVYQNEFHIQE |
| | LEQLYDFYLDKLKNGVYKNRKNNQAELIHNEKEQFMELKTEDQCVLLTEIKKLFVCSPM |
| | QADLTLIGGSKHTGMIAMSSNVTKADFAVIAEDPLGLRNKVIYSHKGEK (SEQ ID NO: 16) |
| KyCas9 | MSQNNNKIYNIGLDIGDASVGWAVVDEHYNLLKRHGKHMWGSRLFTQANTAVERRSSR |
| Kandleria | STRRRYNKRRERIRLLREIMEDMVLDVDPTFFIRLANVSFLDQEDKKDYLKENYHSNYNL |
| vitulina | FIDKDENDKTYYDKYPTIYHLRKHLCESKEKEDPRLIYLALHHIVKYRGNFLYEGQKESM |
| NCBI Reference | DVSNIEDKMIDVLRQFNEINLFEYVEDRKKIDEVLNVLKEPLSKKHKAEKAFALFDTTKD |
| Sequence: | NKAAYKELCAALAGNKENVTKMLKEAELHDEDEKDISFKFSDATFDDAFVEKQPLLGDC |
| WP_0315899691 | VEFIDLLHDIYSWVELQNILGSAHTSEPSISAAMIQRYEDHKNDLKLLKDVIRKYLPKKYF |
| Wild type | EVFRDEKSKKNNYCNYINHPSKTPVDEFYKYIKKLIEKIDDPDVKTILNKIELESFMLKQNS |
| | RTNGAVPYQMQLDELNKILENQSVYYSDLKDNEDKIRSILTFRIPYYFGPLNITKDRQFDW |
| | IIKKEGKENERILPWNANEIVDVDKTADEFIKRMRNFCTYFPDEPVMAKNSLTVSKYEVL |
| | NEINKLRINDHLIKRDMKDKMLHTLFMDHKSISANAMKKWLVKNQYFSNTDDIKIEGFQ |
| | KENACSTSLTPWIDFTKIFGKINESNYDFIEKIIYDVTVFEDKKILRRRLKKEYDLDEEKIKK |
| | ILKLKYSGWSRLSKKLLSGIKTKYKDSTRTPETVLEVMERTNMNLMQVINDEKLGFKKTI |
| | DDANSTSVSGKFSYAEVQELAGSPAIKRGIWQALLIVDEIKKIMKHEPAHVYIEFARNEDE |
| | KERKDSFVNQMLKLYKDYDFEDETEKEANKHLKGEDAKSKIRSERLKLYYTQMGKCMY |
| | TGKSLDIDRLDTYQVDHIVPQSLLKDDSIDNKVLVLSSENQRKLDDLVIPSSIRNKMYGFW |
| | EKLENNKIISPKKFYSLIKTEFNEKDQERFINRQIVETRQITKHVAQIIDNHYENTKVVTVRA |
| | DLSHQFRERYHIYKNRDINDFHHAHDAYIATILGTYIGHRFESLDAKYIYGEYKRIFRNQK |
| | NKGKEMKKNNDGFILNSMRNIYADKDTGEIVWDPNYIDRIKKCFYYKDCFVTKKLEENN |
| | GTFFNVTVLPNDTNSDKDNTLATVPVNKYRSNVNKYGGFSGVNSFIVAIKGKKKKGKKV |
| | IEVNKLTGIPLMYKNADEEIKINYLKQAEDLEEVQIGKEILKNQLIEKDGGLYYIVAPTEIIN |
| | AKQLILNESQTKLVCEIYKAMKYKNYDNLDSEKIIDLYRLLINKMELYYPEYRKQL VKKF |
| | EDRYEQLKVISIEEKCNIIKQILATLHCNSSIGKIMYSDFKISTTIGRLNGRTISLDDISFIAESP |
| | TGMYSKKYKL (SEQ ID NO: 17) |
| EfCas9 | MRLFEEGHTAEDRRLKRTARRRISRRRNRLRYLQAFFEEAMTDLDENFFARLQESFLVPE |
| Enterococcus | DKKWHRHPIFAKLEDEVAYHETYPTIYHLRKKLADSSEQADLRLIYLALAHIVKYRGHFLI |
| faecalis | EGKLSTENTSVKDQFQQFMVIYNQTFVNGESRLVSAPLPESVLIEEELTEKASRTKKSEKV |
| NCBI Reference | LQQFPQEKANGLFGQFLKLMVGNKADFKKVFGLEEEAKITYASESYEEDLEGILAKVGDE |
| Sequence: | YSDVFLAAKNVYDAVELSTILADSDKKSHAKLSSSMIVRFTEHQEDLKKFKRFIRENCPDE |
| WP_016631044.1 | YDNLFKNEQKDGYAGYIAHAGKVSQLKFYQYVKKIIQDIAGAEYFLEKIAQENFLRKQRT |
| Wild type | FDNGVIPHQIHLAELQAIIHRQAAYYPFLKENQEKIEQLVTFRIPYYVGPLSKGDASTFAWL |
| | KRQSEEPIRPWNLQETVDLDQSATAFIERMTNFDTYLPSEKVLPKHSLLYEKFMVFNELTK |
| | ISYTDDRGIKANFSGKEKEKIFDYLFKTRRKVKKKDIIQFYRNEYNTEIVTLSGLEEDQFNA |
| | SFSTYQDLLKCGLTRAELDHPDNAEKLEDIIKILTIFEDRQRIRTQLSTFKGQFSAEVLKKLE |
| | RKHYTGWGRLSKKLINGIYDKESGKTILDYLVKDDGVSKHYNRNFMQLINDSQLSFKNAI |
| | QKAQSSEHEETLSETVNELAGSPAIKKGIYQSLKIVDELVAIMGYAPKRIVVEMARENQTT |
| | STGKRRSIQRLKIVEKAMAEIGSNLLKEQPTTNEQLRDTRLFLYYMQNGKDMYTGDELSL |
| | HRLSHYDIDHIIPQSFMKDDSLDNLVLVGSTENRGKSDDVPSKEVVKDMKAYWEKLYAA |
| | GLISQRKFQRLTKGEQGGLTLEDKAHFIQRQLVETRQITKNVAGILDQRYNAKSKEKKVQI |
| | ITLKASLTSQFRSIFGLYKVREVNDYHHGQDAYLNCVVATTLLKVYPNLAPEFVYGEYPK |
| | FQTFKENKATAKAIIYTNLLRFFTEDEPRFTKDGEILWSNSYLKTIKKELNYHQMNIVKKV |
| | EVQKGGFSKESIKPKGPSNKLIPVKNGLDPQKYGGFDSPVVAYTVLFTHEKGKKPLIKQEI |
| | LGITIMEKTRFEQNPILFLEEKGFLRPRVLMKLPKYTLYEFPEGRRRLLASAKEAQKGNQM |
| | VLPEHLLTLLYHAKQCLLPNQSESLAYVEQHQPEFQEILERVVDFAEVHTLAKSKVQQIV |
| | KLFEANQTADVKEIAASFIQLMQFNAMGAPSTFKFFQKDIERARYTSIKEIFDATIIYQSPTG |
| | LYETRRKVVD (SEQ ID NO: 18) |
| Staphylococcus | KRNYILGLDIGITSVGYGIIDYETRDVIDAGVRLFKEANVENNEGRRSKRGARRLKRRRRH |
| aureus Cas9 | RIQRVKKLLFDYNLLTDHSELSGINPYEARVKGLSQKLSEEEFSAALLHLAKRRGVHNVN |
| | EVEEDTGNELSTKEQISRNSKALEEKYVAELQLERLKKDGEVRGSINRFKTSDYVKEAKQ |
| | LLKVQKAYHQLDQSFIDTYIDLLETRRTYYEGPGEGSPFGWKDIKEWYEMLMGHCTYFP |
| | EELRSVKYAYNADLYNALNDLNNLVITRDENEKLEYYEKFQIIENVFKQKKKPTLKQIAK |
| | EILVNEEDIKGYRVTSTGKPEFTNLKVYHDIKDITARKEIENAELLDQIAKILTIYQSSEDIQ |
| | EELTNLNSELTQEEIEQISNLKGYTGTHNLSLKAINLILDELWHTNDNQIAIFNRLKLVPKK |
| | VDLSQQKEIPTTLVDDFILSPVVKRSFIQSIKVINAIIKKYGLPNDIIIELAREKNSKDAQKMI |
| | NEMQKRNRQTNERIEEIIRTTGKENAKYLIEKIKLHDMQEGKCLYSLEAIPLEDLLNNPEN |
| | YEVDHIIPRSVSFDNSFNNKVLVKQEENSKKGNRTPFQYLSSSDSKISYETFKKHILNLAKG |
| | KGRISKTKKEYLLEERDINRESVQKDFINRNLVDTRYATRGLMNLLRSYFRVNNLDVKVK |
| | SINGGFTSFLRRKWKFKKERNKGYKHHAEDALIIANADFIFKEWKKLDKAKKVMENQMF |
| | EEKQAESMPEIETEQEYKEIFITPHQIKHIKDFKDYKYSHRVDKKPNRELINDTLYSTRKDD |
| | KGNTLIVNNLNGLYDKDNDKLKKLINKSPEKLLMYHHDPQTYQKLKLIMEQYGDEKNPL |
| | YKYYEETGNYLTKYSKKDNGPVIKKIKYYGNKLNAHLDITDDYPNSRNKVVKLSLKPYR |
| | FDVYLDNGVYKFVTVKNLDVIKKENYYEVNSKCYEEAKKLKKISNQAEFIASFYNNDLIK |
| | INGELYRVIGVNNDLLNRIEVNMIDITYREYLENMNDKRPPRIIKTIASKTQSIKKYSTDILG |
| | NLYEVKSKKHPQIIKKG (SEQ ID NO: 19) |
| Geobacillus | MKYKIGLDIGITSIGWAVINLDIPRIEDLGVRIFDRAENPKTGESLALPRRLARSARRRLRRR |
| thermodenitrificans | KHRLERIRRLFVREGILTKEELNKLFEKKHEIDVWQLRVEALDRKLNNDELARILLHLAKR |
| Cas9 | RGFRSNRKSERTNKENSTMLKHIEENQSILSSYRTVAEMVVKDPKFSLHKRNKEDNYTNT |
| | VARDDLEREIKLIFAKQREYGNIVCTEAFEHEYISIWASQRPFASKDDIEKKVGFCTFEPKE |
| | KRAPKATYTFQSFTVWEHINKLRLVSPGGIRALTDDERRLIYKQAFHKNKITFHDVRTLLN |
| | LPDDTRFKGLLYDRNTTLKENEKVRFLELGAYHKIRKAIDSVYGKGAAKSFRPIDEDTFG |
| | YALTMEKDDTDIRSYLRNEYEQNGKRMENLADKVYDEELIEELLNLSFSKFGHLSLKALR |
| | NILPYMEQGEVYSTACERAGYTFTGPKKKQKTVLLPNIPPIANPVVMRALTQARKVVNAII |
| | KKYGSPVSIHIELARELSQSFDERRKMQKEQEGNRKKNETAIRQLVEYGLTLNPTGLDIVK |
| | FKLWSEQNGKCAYSLQPIEIERLLEPGYTEVDHVIPYSRSLDDSYTNKVLVLTKENREKGN |
| | RTPAEYLGLGSERWQQFETFVLINKQFSKKKRDRLLRLHYDENEENEFKNRNLNDTRYIS |
| | RFLANFIREHLKFADSDDKQKVYTVNGRITAHLRSRWNENKNREESNLHHAVDAAIVAC |
| | TTPSDIARVTAFYQRREQNKELSKKTDPQFPQPWPHFADELQARLSKNPKESIKALNLGN |
| | YDNEKLESLQPVFVSRMPKRSITGAAHQETLRRYIGIDERSGKIQTVVKKKLSEIQLDKTG |
| | HFPMYGKESDPRTYEAIRQRLLEHNNDPKKAFQEPLYKPKKNGELGPIIRTIKIIDTTNQVIP |
| | LNDGKTVAYNSNIVRVDVFEKDGKYYCVPIYTIDMMKGILPNKAIEPNKPYSEWKEMTE |
| | DYTFRFSLYPNDLIRIEFPREKTIKTAVGEEIKIKDLFAYYQTIDSSNGGLSLVSHDNNFSLR |
| | SIGSRTLKRFEKYQVDVLGNIYKVRGEKRVGVASSSHSKAGETIRPL (SEQ ID NO: 20) |
| ScCas9 | MEKKYSIGLDIGTNSVGWAVITDDYKVPSKKFKVLGNTNRKSIKKNLMGALLFDSGETAE |
| S. canis | ATRLKRTARRRYTRRKNRIRYLQEIFANEMAKLDDSFFQRLEESFLVEEDKKNERHPIFGN |
| 1375 AA | LADEVAYHRNYPTIYHLRKKLADSPEKADLRLIYLALAHIIKFRQHFLIEGKLNAENSDVA |
| 159.2 kDa | KLFYQLIQTYNQLFEESPLDEIEVDAKGILSARLSKSKRLEKLIAVFPNEKKNGLEGNIIALA |
| | LGLTPNFKSNFDLTEDAKLQLSKDTYDDDLDELLGQIGDQYADLFSAAKNLSDAILLSDIL |
| | RSNSEVTKAPLSASMVKRYDEHHQDLALLKTLVRQQFPEKYAEIFKDDTKNGYAGYVGI |
| | GIKHRKRTTKLATQEEFYKFIKPILEKMDGAEELLAKLNRDDLLRKQRTFDNGSIPHQIHL |
| | KELHAILRRQEEFYPFLKENREKIEKILTFRIPYYVGPLARGNSRFAWLTRKSEEATTPWNF |
| | EEVVDKGASAQSFIERMTNFDEQLPNKKVLPKHSLLYEYFTVYNELTKVKYVTERMRKP |
| | EFLSGEQKKAIVDLLFKTNRKVTVKQLKEDYFKKIECFDSVEIIGVEDRENASLGTYHDLL |
| | KIIKDKDFLDNEENEDILEDIVLTLTLFEDREMIEERLKTYAHLEDDKVMKQLKRRHYTG |
| | WGRLSRKMINGIRDKQSGKTILDFLKSDGFSNRNFMQLIHDDSLTFKEEIEKAQVSGQGDS |
| | LHEQIADLAGSPAIKKGILQTVKIVDELVKVMGHKPENIVIEMARENQTTTKGLQQSRERK |
| | KRIEEGIKELESQILKENPVENTQLQNEKLYLYYLQNGRDMYVDQELDINRLSDYDVDHI |
| | VPQSFIKDDSIDNKVLTRSVENRGKSDNVPSEEVVKKMKNYWRQLLNAKLITQRKFDNLT |
| | KAERGGLSEADKAGFIKRQLVETRQITKHVARILDSRMNTKRDKNDKPIREVKVITLKSKL |
| | VSDFRKDFQLYKVRDINNYHHAHDAYLNAVVGTALIKKYPKLESEFVYGDYKVYDVRK |
| | MIAKSEQEIGKATAKRFFYSNIMNFFKTEVKLANGEIRKRPLIETNGETGEVVWNKEKDFA |
| | TVRKVLAMPQVNIVKKTEVQTGGFSKESILSKRESAKLIPRKKGWDTRKYGGFGSPTVAY |
| | SILVVAKVEKGKAKKLKSVKVLVGITIMEKGSYEKDPIGFLEAKGYKDIKKELIFKLPKYS |
| | LFELENGRRRMLASATELQKANELVLPQHLVRLLYYTQNISATTGSNNLGYIEQHREEFK |
| | EIFEKIIDFSEKYILKNKVNSNLKSSFDEQFAVSDSILLSNSFVSLLKYTSFGASGGFTFLDLD |
| | VKQGRLRYQTVTEVLDATLIYQSITGLYETRTDLSQLGGD (SEQ ID NO: 21) |

The prime editors utilized in the methods and compositions described herein may include any of the above Cas9 ortholog sequences, or any variants thereof having at least 80%, at least 85%, at least 90%, at least 95%, or at least 99% sequence identity thereto.

The napDNAbp may include any suitable homologs and/or orthologs or naturally occurring enzymes, such as, Cas9, Cas9 homologs and/or orthologs have been described in various species, including, but not limited to, S. pyogenes and S. thermophilus. Preferably, the Cas moiety is configured (e.g., mutagenized, recombinantly engineered, or otherwise obtained from nature) as a nickase, i.e., capable of cleaving only a single strand of the target double-stranded DNA. Additional suitable Cas9 nucleases and sequences will be apparent to those of skill in the art based on this disclosure, and such Cas9 nucleases and sequences include Cas9 sequences from the organisms and loci disclosed in Chylinski, Rhun. and Charpentier. “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain; that is, the Cas9 is a nickase. In some embodiments, the Cas9 protein comprises an amino acid sequence that is at least 80% identical to the amino acid sequence of a Cas9 protein as provided by any one of the variants of Table 3. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs in the above tables.

### C. Dead Cas9 Variant

In certain embodiments, the prime editors utilized in the methods and compositions described herein may include a dead Cas9, e.g., dead SpCas9, which has no nuclease activity due to one or more mutations that inactive both nuclease domains of Cas9, namely the RuvC domain (which cleaves the non-protospacer DNA strand) and HNH domain (which cleaves the protospacer DNA strand). The nuclease inactivation may be due to one or mutations that result in one or more substitutions and/or deletions in the amino acid sequence of the encoded protein, or any variants thereof having at least 80%, at least 85%, at least 90%, at least 95%, or at least 99% sequence identity thereto.

As used herein, the term “dCas9” refers to a nuclease-inactive Cas9 or nuclease-dead Cas9, or a functional fragment thereof, and embraces any naturally occurring dCas9 from any organism, any naturally-occurring dCas9 equivalent or functional fragment thereof, any engineered dCas9 variant or functional fragment thereof, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and method for making dCas9 proteins are further described herein and/or are described in the art and are incorporated herein by reference.

In other embodiments, dCas9 corresponds to, or comprises in part or in whole, a Cas9 amino acid sequence having one or more mutations that inactivate the Cas9 nuclease activity. In other embodiments, Cas9 variants having mutations other than D10A and H840A are provided which may result in the full or partial inactivation of the endogenous Cas9 nuclease activity (e.g., nCas9 or dCas9, respectively). Such mutations, by way of example, include other amino acid substitutions at D10 and H840, or other substitutions within the nuclease domains of Cas9 (e.g., substitutions in the HNH nuclease subdomain and/or the RuvC1 subdomain) with reference to a wild type sequence such as Cas9 from Streptococcus pyogenes (NCBI Reference Sequence: NC_017053.1). In some embodiments, variants or homologues of Cas9 (e.g., variants of Cas9 from Streptococcus pyogenes (NCBI Reference Sequence: NC_017053.1 (SEQ ID NO: 4))) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of dCas9 (e.g., variants of NCBI Reference Sequence: NC_017053.1 (SEQ ID NO: 4)) are provided having amino acid sequences which are shorter, or longer than NC_017053.1 (SEQ ID NO: 4) by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In one embodiment, the dead Cas9 may be based on the canonical SpCas9 sequence of Q99ZW2 and may have the following sequence, which comprises a D10X and an H810X, wherein X may be any amino acid, substitutions (underlined and bolded), or a variant be variant of SEQ ID NO: 40 having at least 80%, at least 85%, at least 90%, at least 95%, or at least 99% sequence identity thereto.

In one embodiment, the dead Cas9 may be based on the canonical SpCas9 sequence of Q99ZW2 and may have the following sequence, which comprises a D10A and an H810A substitutions (underlined and bolded), or be a variant of SEQ ID NO: 23 having at least 80%, at least 85%, at least 90%, at least 95%, or at least 99% sequence identity thereto.

| | | SEQ |
|---|---|---|
| Description | Sequence | ID NO: |
| dead Cas9 or | MDKKYSIGLXIGTNSVGWAVITDEYKVPSKKFKVLGNTDRHSIKKNLIGALLEDSG | 22 |
| dCas9 | ETAEATRLKRTARRRYTRRKNRICYLQEIFSNEMAKVDDSFFHRLEESFLVEEDKK | |
| Streptococcus | HERHPIFGNIVDEVAYHEKYPTIYHLRKKLVDSTDKADLRLIYLALAHMIKERGHFL | |
| pyogenes | IEGDLNPDNSDVDKLFIQLVQTYNQLFEENPINASGVDAKAILSARLSKSRRLENLIA | |
| Q99ZW2 Cas9 | QLPGEKKNGLFGNLIALSLGLTPNFKSNFDLAEDAKLQLSKDTYDDDLDNLLAQIG | |
| with D10X and | DQYADLFLAAKNLSDAILLSDILRVNTEITKAPLSASMIKRYDEHHQDLTLLKALVR | |
| H810X | QQLPEKYKEIFFDQSKNGYAGYIDGGASQEEFYKFIKPILEKMDGTEELLVKLNRED | |
| Where “X” is | LLRKQRTFDNGSIPHQIHLGELHAILRRQEDFYPFLKDNREKIEKILTFRIPYYVGPLA | |
| any amino acid | RGNSRFAWMTRKSEETITPWNFEEVVDKGASAQSFIERMTNFDKNLPNEKVLPKHS | |
| | LLYEYFTVYNELTKVKYVTEGMRKPAFLSGEQKKAIVDLLFKTNRKVTVKQLKED | |
| | YFKKIECFDSVEISGVEDRENASLGTYHDLLKIIKDKDFLDNEENEDILEDIVLTLTLF | |
| | EDREMIEERLKTYAHLEDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKELGSQI | |
| | LKEHPVENTQLQNEKLYLYYLQNGRDMYVDQELDINRLSDYDVDXIVPQSFLKDD | |
| | SIDNKVLTRSDKNRGKSDNVPSEEVVKKMKNYWRQLLNAKLITQRKEDNLTKAER | |
| | GGLSELDKAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGDYKVYD | |
| | VRKMIAKSEQEIGKATAKYFFYSNIMNFFKTEITLANGEIRKRPLIETNGETGEIVWD | |
| | KGRDFATVRKVLSMPQVNIVKKTEVQTGGFSKESILPKRNSDKLIARKKDWDPKK | |
| | YGGFDSPTVAYSVLVVAKVEKGKSKKLKSVKELLGITIMERSSFEKNPIDFLEAKG | |
| | YKEVKKDLIKLPKYSLFELENGRKRMLASAGELQKGNELALPSKYVNFLYLASHY | |
| | EKLKGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYET | |
| | RIDLSQLGGD | |
| dead Cas9 or | MDKKYSIGLAIGTNSVGWAVITDEYKVPSKKFKVLGNTDRHSIKKNLIGALLEDSG | 23 |
| dCas9 | ETAEATRLKRTARRRYTRRKNRICYLQEIFSNEMAKVDDSFFHRLEESFLVEEDKK | |
| Streptococcus | HERHPIFGNIVDEVAYHEKYPTIYHLRKKLVDSTDKADLRLIYLALAHMIKERGHFL | |
| pyogenes | IEGDLNPDNSDVDKLFIQLVQTYNQLFEENPINASGVDAKAILSARLSKSRRLENLIA | |
| Q99ZW2 Cas9 | QLPGEKKNGLFGNLIALSLGLTPNFKSNFDLAEDAKLQLSKDTYDDDLDNLLAQIG | |
| with D10A and | DQYADLFLAAKNLSDAILLSDILRVNTEITKAPLSASMIKRYDEHHQDLTLLKALVR | |
| H810A | QQLPEKYKEIFFDQSKNGYAGYIDGGASQEEFYKFIKPILEKMDGTEELLVKLNRED | |
| | LLRKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHS | |
| | LLYEYFTVYNELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDILEDIVLTLTLF | |
| | EDREMIEERLKTYAHLEDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKELGSQI | |
| | LKEHPVENTQLQNEKLYLYYLQNGRDMYVDQELDINRLSDYDVDAIVPQSFLKDD | |
| | SIDNKVLTRSDKNRGKSDNVPSEEVVKKMKNYWRQLLNAKLITQRKFDNLTKAER | |
| | GGLSELDKAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGDYKVYD | |
| | VRKMIAKSEQEIGKATAKYFFYSNIMNFFKTEITLANGEIRKRPLIETNGETGEIVWD | |
| | KGRDFATVRKVLSMPQVNIVKKTEVQTGGFSKESILPKRNSDKLIARKKDWDPKK | |
| | YGGFDSPTVAYSVLVVAKVEKGKSKKLKSVKELLGITIMERSSFEKNPIDFLEAKG | |
| | YKEVKKDLIKLPKYSLFELENGRKRMLASAGELQKGNELALPSKYVNFLYLASHY | |
| | EKLKGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYET | |
| | RIDLSQLGGD | |

### D. Cas9 Nickase Variant

In one embodiment, the prime editors utilized in the methods and compositions described herein comprise a Cas9 nickase. The term “Cas9 nickase” or “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, of H983A, D986A, or E762A, or a combination thereof.

In various embodiments, the Cas9 nickase can have a mutation in the RuvC nuclease domain and have one of the following amino acid sequences, or a variant thereof having an amino acid sequence that has at least 80%, at least 85%, at least 90%, at least 95%, or at least 99% sequence identity thereto.

| | | SEQ ID |
|---|---|---|
| Description | Sequence | NO: |
| Cas9 nickase | MDKKYSIGLXIGTNSVGWAVITDEYKVPSKKFKVLGNTDRHSIKKNLIGALLEDS | 24 |
| Streptococcus | GETAEATRLKRTARRRYTRRKNRICYLQEIFSNEMAKVDDSFFHRLEESFLVEEDK | |
| pyogenes | KHERHPIFGNIVDEVAYHEKYPTIYHLRKKLVDSTDKADLRLIYLALAHMIKERGH | |
| Q99ZW2 Cas9 | FLIEGDLNPDNSDVDKLFIQLVQTYNQLFEENPINASGVDAKAILSARLSKSRRLEN | |
| with D10X, | LIAQLPGEKKNGLFGNLIALSLGLTPNFKSNFDLAEDAKLQLSKDTYDDDLDNLL | |
| wherein X is any | AQIGDQYADLFLAAKNLSDAILLSDILRVNTEITKAPLSASMIKRYDEHHQDLTLL | |
| alternate amino | KALVRQQLPEKYKEIFFDQSKNGYAGYIDGGASQEEFYKFIKPILEKMDGTEELLV | |
| acid | KLNREDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLP | |
| | NEKVLPKHSLLYEYFTVYNELTKVKYVTEGMRKPAFLSGEQKKAIVDLLEKTNR | |
| | KVTVKQLKEDYFKKIECFDSVEISGVEDRENASLGTYHDLLKIIKDKDFLDNEENE | |
| | DILEDIVLTLTLFEDREMIEERLKTYAHLEDDKVMKQLKRRRYTGWGRLSRKLIN | |
| | GIRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSR | |
| | ERMKRIEEGIKELGSQILKEHPVENTQLQNEKLYLYYLQNGRDMYVDQELDINRL | |
| | SDYDVDHIVPQSFLKDDSIDNKVLTRSDKNRGKSDNVPSEEVVKKMKNYWRQLL | |
| | NAKLITQRKFDNLTKAERGGLSELDKAGFIKRQLVETRQITKHVAQILDSRMNTK | |
| | YDENDKLIREVKVITLKSKLVSDFRKDFQFYKVREINNYHHAHDAYLNAVVGTA | |
| | LIKKYPKLESEFVYGDYKVYDVRKMIAKSEQEIGKATAKYFFYSNIMNFFKTEITL | |
| | ANGEIRKRPLIETNGETGEIVWDKGRDFATVRKVLSMPQVNIVKKTEVQTGGFSK | |
| | ESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPAAFKYEDTTI | |
| | DRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLEDS | 25 |
| Streptococcus | GETAEATRLKRTARRRYTRRKNRICYLQEIFSNEMAKVDDSFFHRLEESFLVEEDK | |
| pyogenes | KHERHPIFGNIVDEVAYHEKYPTIYHLRKKLVDSTDKADLRLIYLALAHMIKERGH | |
| Q99ZW2 Cas9 | FLIEGDLNPDNSDVDKLFIQLVQTYNQLFEENPINASGVDAKAILSARLSKSRRLEN | |
| with E762X, | LIAQLPGEKKNGLFGNLIALSLGLTPNFKSNFDLAEDAKLQLSKDTYDDDLDNLL | |
| wherein X is any | AQIGDQYADLFLAAKNLSDAILLSDILRVNTEITKAPLSASMIKRYDEHHQDLTLL | |
| alternate amino | KALVRQQLPEKYKEIFFDQSKNGYAGYIDGGASQEEFYKFIKPILEKMDGTEELLV | |
| acid | KLNREDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLP | |
| | NEKVLPKHSLLYEYFTVYNELTKVKYVTEGMRKPAFLSGEQKKAIVDLLEKTNR | |
| | KVTVKQLKEDYFKKIECFDSVEISGVEDRENASLGTYHDLLKIIKDKDFLDNEENE | |
| | DILEDIVLTLTLFEDREMIEERLKTYAHLEDDKVMKQLKRRRYTGWGRLSRKLIN | |
| | GIRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIXMARENQTTQKGQKNSR | |
| | ERMKRIEEGIKELGSQILKEHPVENTQLQNEKLYLYYLQNGRDMYVDQELDINRL | |
| | SDYDVDHIVPQSFLKDDSIDNKVLTRSDKNRGKSDNVPSEEVVKKMKNYWRQLL | |
| | NAKLITQRKFDNLTKAERGGLSELDKAGFIKRQLVETRQITKHVAQILDSRMNTK | |
| | YDENDKLIREVKVITLKSKLVSDFRKDFQFYKVREINNYHHAHDAYLNAVVGTA | |
| | LIKKYPKLESEFVYGDYKVYDVRKMIAKSEQEIGKATAKYFFYSNIMNFFKTEITL | |
| | ANGEIRKRPLIETNGETGEIVWDKGRDFATVRKVLSMPQVNIVKKTEVQTGGFSK | |
| | ESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPAAFKYFDTTI | |
| | DRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLEDS | 26 |
| Streptococcus | GETAEATRLKRTARRRYTRRKNRICYLQEIFSNEMAKVDDSFFHRLEESFLVEEDK | |
| pyogenes | KHERHPIFGNIVDEVAYHEKYPTIYHLRKKLVDSTDKADLRLIYLALAHMIKFRGH | |
| Q99ZW2 Cas9 | FLIEGDLNPDNSDVDKLFIQLVQTYNQLFEENPINASGVDAKAILSARLSKSRRLEN | |
| with H983X, | LIAQLPGEKKNGLFGNLIALSLGLTPNFKSNFDLAEDAKLQLSKDTYDDDLDNLL | |
| wherein X is any | AQIGDQYADLFLAAKNLSDAILLSDILRVNTEITKAPLSASMIKRYDEHHQDLTLL | |
| alternate amino | KALVRQQLPEKYKEIFFDQSKNGYAGYIDGGASQEEFYKFIKPILEKMDGTEELLV | |
| acid | KLNREDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLP | |
| | NEKVLPKHSLLYEYFTVYNELTKVKYVTEGMRKPAFLSGEQKKAIVDLLFKTNR | |
| | KVTVKQLKEDYFKKIECFDSVEISGVEDRFNASLGTYHDLLKIIKDKDFLDNEENE | |
| | DILEDIVLTLTLFEDREMIEERLKTYAHLEDDKVMKQLKRRRYTGWGRLSRKLIN | |
| | GIRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSR | |
| | ERMKRIEEGIKELGSQILKEHPVENTQLQNEKLYLYYLQNGRDMYVDQELDINRL | |
| | SDYDVDHIVPQSFLKDDSIDNKVLTRSDKNRGKSDNVPSEEVVKKMKNYWRQLL | |
| | NAKLITQRKFDNLTKAERGGLSELDKAGFIKRQLVETRQITKHVAQILDSRMNTK | |
| | YDENDKLIREVKVITLKSKLVSDFRKDFQFYKVREINNYHXAHDAYLNAVVGTA | |
| | LIKKYPKLESEFVYGDYKVYDVRKMIAKSEQEIGKATAKYFFYSNIMNFFKTEITL | |
| | ANGEIRKRPLIETNGETGEIVWDKGRDFATVRKVLSMPQVNIVKKTEVQTGGFSK | |
| | ESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPAAFKYEDTTI | |
| | DRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLEDS | 27 |
| Streptococcus | GETAEATRLKRTARRRYTRRKNRICYLQEIFSNEMAKVDDSFFHRLEESFLVEEDK | |
| pyogenes | KHERHPIFGNIVDEVAYHEKYPTIYHLRKKLVDSTDKADLRLIYLALAHMIKERGH | |
| Q99ZW2 Cas9 | FLIEGDLNPDNSDVDKLFIQLVQTYNQLFEENPINASGVDAKAILSARLSKSRRLEN | |
| with D986X, | LIAQLPGEKKNGLFGNLIALSLGLTPNFKSNFDLAEDAKLQLSKDTYDDDLDNLL | |
| wherein X is any | AQIGDQYADLFLAAKNLSDAILLSDILRVNTEITKAPLSASMIKRYDEHHQDLTLL | |
| alternate amino | KALVRQQLPEKYKEIFFDQSKNGYAGYIDGGASQEEFYKFIKPILEKMDGTEELLV | |
| acid | KLNREDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLP | |
| | NEKVLPKHSLLYEYFTVYNELTKVKYVTEGMRKPAFLSGEQKKAIVDLLFKTNR | |
| | KVTVKQLKEDYFKKIECFDSVEISGVEDRFNASLGTYHDLLKIIKDKDFLDNEENE | |
| | DILEDIVLTLTLFEDREMIEERLKTYAHLEDDKVMKQLKRRRYTGWGRLSRKLIN | |
| | GIRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSR | |
| | ERMKRIEEGIKELGSQILKEHPVENTQLQNEKLYLYYLQNGRDMYVDQELDINRL | |
| | SDYDVDHIVPQSFLKDDSIDNKVLTRSDKNRGKSDNVPSEEVVKKMKNYWRQLL | |
| | NAKLITQRKFDNLTKAERGGLSELDKAGFIKRQLVETRQITKHVAQILDSRMNTK | |
| | YDENDKLIREVKVITLKSKLVSDFRKDFQFYKVREINNYHHAHXAYLNAVVGTA | |
| | LIKKYPKLESEFVYGDYKVYDVRKMIAKSEQEIGKATAKYFFYSNIMNFFKTEITL | |
| | ANGEIRKRPLIETNGETGEIVWDKGRDFATVRKVLSMPQVNIVKKTEVQTGGFSK | |
| | ESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPAAFKYEDTTI | |
| | DRKRYTSTKEVLDATLIHQSITGLYETRIDLSQLGGD | |
| Cas9 nickase | MDKKYSIGLAIGTNSVGWAVITDEYKVPSKKFKVLGNTDRHSIKKNLIGALLEDS | 28 |
| Streptococcus | GETAEATRLKRTARRRYTRRKNRICYLQEIFSNEMAKVDDSFFHRLEESELVEEDK | |
| pyogenes | KHERHPIFGNIVDEVAYHEKYPTIYHLRKKLVDSTDKADLRLIYLALAHMIKERGH | |
| Q99ZW2 Cas9 | FLIEGDLNPDNSDVDKLFIQLVQTYNQLFEENPINASGVDAKAILSARLSKSRRLEN | |
| with D10A | LIAQLPGEKKNGLFGNLIALSLGLTPNFKSNFDLAEDAKLQLSKDTYDDDLDNLL | |
| | AQIGDQYADLFLAAKNLSDAILLSDILRVNTEITKAPLSASMIKRYDEHHQDLTLL | |
| | KALVRQQLPEKYKEIFFDQSKNGYAGYIDGGASQEEFYKFIKPILEKMDGTEELLV | |
| | KLNREDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLP | |
| | NEKVLPKHSLLYEYFTVYNELTKVKYVTEGMRKPAFLSGEQKKAIVDLLEKTNR | |
| | KVTVKQLKEDYFKKIECFDSVEISGVEDRENASLGTYHDLLKIIKDKDFLDNEENE | |
| | DILEDIVLTLTLFEDREMIEERLKTYAHLEDDKVMKQLKRRRYTGWGRLSRKLIN | |
| | GIRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSR | |
| | ERMKRIEEGIKELGSQILKEHPVENTQLQNEKLYLYYLQNGRDMYVDQELDINRL | |
| | SDYDVDHIVPQSFLKDDSIDNKVLTRSDKNRGKSDNVPSEEVVKKMKNYWRQLL | |
| | NAKLITQRKFDNLTKAERGGLSELDKAGFIKRQLVETRQITKHVAQILDSRMNTK | |
| | YDENDKLIREVKVITLKSKLVSDFRKDFQFYKVREINNYHHAHDAYLNAVVGTA | |
| | LIKKYPKLESEFVYGDYKVYDVRKMIAKSEQEIGKATAKYFFYSNIMNFFKTEITL | |
| | ANGEIRKRPLIETNGETGEIVWDKGRDFATVRKVLSMPQVNIVKKTEVQTGGFSK | |
| | ESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPAAFKYEDTTI | |
| | DRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLEDS | 29 |
| Streptococcus | GETAEATRLKRTARRRYTRRKNRICYLQEIFSNEMAKVDDSFFHRLEESFLVEEDK | |
| pyogenes | KHERHPIFGNIVDEVAYHEKYPTIYHLRKKLVDSTDKADLRLIYLALAHMIKFRGH | |
| Q99ZW2 Cas9 | FLIEGDLNPDNSDVDKLFIQLVQTYNQLFEENPINASGVDAKAILSARLSKSRRLEN | |
| with E762A | LIAQLPGEKKNGLFGNLIALSLGLTPNFKSNFDLAEDAKLQLSKDTYDDDLDNLL | |
| | AQIGDQYADLFLAAKNLSDAILLSDILRVNTEITKAPLSASMIKRYDEHHQDLTLL | |
| | KALVRQQLPEKYKEIFFDQSKNGYAGYIDGGASQEEFYKFIKPILEKMDGTEELLV | |
| | KLNREDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNEDKNLP | |
| | NEKVLPKHSLLYEYFTVYNELTKVKYVTEGMRKPAFLSGEQKKAIVDLLEKTNR | |
| | KVTVKQLKEDYFKKIECFDSVEISGVEDRENASLGTYHDLLKIIKDKDFLDNEENE | |
| | DILEDIVLTLTLFEDREMIEERLKTYAHLEDDKVMKQLKRRRYTGWGRLSRKLIN | |
| | GIRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIAMARENQTTQKGQKNSR | |
| | ERMKRIEEGIKELGSQILKEHPVENTQLQNEKLYLYYLQNGRDMYVDQELDINRL | |
| | SDYDVDHIVPQSFLKDDSIDNKVLTRSDKNRGKSDNVPSEEVVKKMKNYWRQLL | |
| | NAKLITQRKFDNLTKAERGGLSELDKAGFIKRQLVETRQITKHVAQILDSRMNTK | |
| | YDENDKLIREVKVITLKSKLVSDFRKDFQFYKVREINNYHHAHDAYLNAVVGTA | |
| | LIKKYPKLESEFVYGDYKVYDVRKMIAKSEQEIGKATAKYFFYSNIMNFFKTEITL | |
| | ANGEIRKRPLIETNGETGEIVWDKGRDFATVRKVLSMPQVNIVKKTEVQTGGFSK | |
| | ESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPAAFKYEDTTI | |
| | DRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLEDS | 30 |
| Streptococcus | GETAEATRLKRTARRRYTRRKNRICYLQEIFSNEMAKVDDSFFHRLEESFLVEEDK | |
| pyogenes | KHERHPIFGNIVDEVAYHEKYPTIYHLRKKLVDSTDKADLRLIYLALAHMIKERGH | |
| Q99ZW2 Cas9 | FLIEGDLNPDNSDVDKLFIQLVQTYNQLFEENPINASGVDAKAILSARLSKSRRLEN | |
| with H983A | LIAQLPGEKKNGLFGNLIALSLGLTPNFKSNFDLAEDAKLQLSKDTYDDDLDNLL | |
| | AQIGDQYADLFLAAKNLSDAILLSDILRVNTEITKAPLSASMIKRYDEHHQDLTLL | |
| | KALVRQQLPEKYKEIFFDQSKNGYAGYIDGGASQEEFYKFIKPILEKMDGTEELLV | |
| | KLNREDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLP | |
| | NEKVLPKHSLLYEYFTVYNELTKVKYVTEGMRKPAFLSGEQKKAIVDLLFKTNR | |
| | KVTVKQLKEDYFKKIECFDSVEISGVEDRFNASLGTYHDLLKIIKDKDELDNEENE | |
| | DILEDIVLTLTLFEDREMIEERLKTYAHLEDDKVMKQLKRRRYTGWGRLSRKLIN | |
| | GIRDKQSGKTILDELKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSR | |
| | ERMKRIEEGIKELGSQILKEHPVENTQLQNEKLYLYYLQNGRDMYVDQELDINRL | |
| | SDYDVDHIVPQSFLKDDSIDNKVLTRSDKNRGKSDNVPSEEVVKKMKNYWRQLL | |
| | NAKLITQRKFDNLTKAERGGLSELDKAGFIKRQLVETRQITKHVAQILDSRMNTK | |
| | YDENDKLIREVKVITLKSKLVSDFRKDFQFYKVREINNYHAAHDAYLNAVVGTA | |
| | LIKKYPKLESEFVYGDYKVYDVRKMIAKSEQEIGKATAKYFFYSNIMNFFKTEITL | |
| | ANGEIRKRPLIETNGETGEIVWDKGRDFATVRKVLSMPQVNIVKKTEVQTGGFSK | |
| | ESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPAAFKYEDTTI | |
| | DRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLEDS | 31 |
| Streptococcus | GETAEATRLKRTARRRYTRRKNRICYLQEIFSNEMAKVDDSFFHRLEESFLVEEDK | |
| pyogenes | KHERHPIFGNIVDEVAYHEKYPTIYHLRKKLVDSTDKADLRLIYLALAHMIKFRGH | |
| Q99ZW2 Cas9 | FLIEGDLNPDNSDVDKLFIQLVQTYNQLFEENPINASGVDAKAILSARLSKSRRLEN | |
| with D986A | LIAQLPGEKKNGLFGNLIALSLGLTPNFKSNFDLAEDAKLQLSKDTYDDDLDNLL | |
| | AQIGDQYADLFLAAKNLSDAILLSDILRVNTEITKAPLSASMIKRYDEHHQDLTLL | |
| | KALVRQQLPEKYKEIFFDQSKNGYAGYIDGGASQEEFYKFIKPILEKMDGTEELLV | |
| | KLNREDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNEDKNLP | |
| | NEKVLPKHSLLYEYFTVYNELTKVKYVTEGMRKPAFLSGEQKKAIVDLLEKTNR | |
| | KVTVKQLKEDYFKKIECFDSVEISGVEDRENASLGTYHDLLKIIKDKDFLDNEENE | |
| | DILEDIVLTLTLFEDREMIEERLKTYAHLEDDKVMKQLKRRRYTGWGRLSRKLIN | |
| | GIRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSR | |
| | ERMKRIEEGIKELGSQILKEHPVENTQLQNEKLYLYYLQNGRDMYVDQELDINRL | |
| | SDYDVDHIVPQSFLKDDSIDNKVLTRSDKNRGKSDNVPSEEVVKKMKNYWRQLL | |
| | NAKLITQRKFDNLTKAERGGLSELDKAGFIKRQLVETRQITKHVAQILDSRMNTK | |
| | YDENDKLIREVKVITLKSKLVSDFRKDFQFYKVREINNYHHAHAAYLNAVVGTA | |
| | LIKKYPKLESEFVYGDYKVYDVRKMIAKSEQEIGKATAKYFFYSNIMNFFKTEITL | |
| | ANGEIRKRPLIETNGETGEIVWDKGRDFATVRKVLSMPQVNIVKKTEVQTGGFSK | |
| | ESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEITEQIS | |
| | EFSKRVILADANLDKVLSAYNKHRDKPIREQAENIIHLFTLTNLGAPAAFKYEDTTI | |
| | DRKRYTSTKEVLDATLIHQSITGLYETRIDLSQLGGD | |

In another embodiment, the Cas9 nickase comprises a mutation in the HNH domain which inactivates the HNH nuclease activity. For example, mutations in histidine (Q) 840 or asparagine (R) 863 have been reported as loss-of-function mutations of the HNH nuclease domain and the creation of a functional Cas9 nickase (e.g., Nishimasu et al., “Crystal structure of Cas9 in complex with guide RNA and target DNAS” Cell 156(5), 935-949, which is incorporated herein by reference). Thus, nickase mutations in the HNH domain could include H84KX and R863X, wherein X is any amino acid other than the wild type amino acid. In certain embodiments, the nickase could be H840A or R863A or a combination thereof.

In various embodiments, the Cas9 nickase can have a mutation in the HNH nuclease domain and have one of the following amino acid sequences, or a variant thereof having an amino acid sequence that has at least 80%, at least 85%, at least 90%, at least 95%, or at least 99% sequence identity thereto.

| | | SEQ |
|---|---|---|
| Description | Sequence | ID NO: |
| Cas9 nickase | MDKKYSIGLDIGTNSVGWAVITDEYKVPSKKFKVLGNTDRHSIKKNLIGALLEDSG | 32 |
| Streptococcus | ETAEATRLKRTARRRYTRRKNRICYLQEIFSNEMAKVDDSFFHRLEESFLVEEDKK | |
| pyogenes | HERHPIFGNIVDEVAYHEKYPTIYHLRKKLVDSTDKADLRLIYLALAHMIKFRGHFL | |
| Q99ZW2 Cas9 | IEGDLNPDNSDVDKLFIQLVQTYNQLFEENPINASGVDAKAILSARLSKSRRLENLIA | |
| with H840X, | QLPGEKKNGLFGNLIALSLGLTPNFKSNFDLAEDAKLQLSKDTYDDDLDNLLAQIG | |
| wherein X is any | DQYADLFLAAKNLSDAILLSDILRVNTEITKAPLSASMIKRYDEHHQDLTLLKALVR | |
| alternate amino | QQLPEKYKEIFFDQSKNGYAGYIDGGASQEEFYKFIKPILEKMDGTEELLVKLNRED | |
| acid | LLRKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHS | |
| | LLYEYFTVYNELTKVKYVTEGMRKPAFLSGEQKKAIVDLLFKTNRKVTVKQLKED | |
| | YFKKIECFDSVEISGVEDRENASLGTYHDLLKIIKDKDFLDNEENEDILEDIVLTLTLF | |
| | EDREMIEERLKTYAHLEDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKELGSQI | |
| | LKEHPVENTQLQNEKLYLYYLQNGRDMYVDQELDINRLSDYDVDXIVPQSFLKDD | |
| | SIDNKVLTRSDKNRGKSDNVPSEEVVKKMKNYWRQLLNAKLITQRKFDNLTKAER | |
| | GGLSELDKAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGDYKVYD | |
| | VRKMIAKSEQEIGKATAKYFFYSNIMNFFKTEITLANGEIRKRPLIETNGETGEIVWD | |
| | KGRDFATVRKVLSMPQVNIVKKTEVQTGGFSKESILPKRNSDKLIARKKDWDPKK | |
| | YGGFDSPTVAYSVLVVAKVEKGKSKKLKSVKELLGITIMERSSFEKNPIDFLEAKG | |
| | YKEVKKDLIIKLPKYSLFELENGRKRMLASAGELQKGNELALPSKYVNFLYLASHY | |
| | EKLKGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYET | |
| | RIDLSQLGGD | |
| Cas9 nickase | MDKKYSIGLDIGTNSVGWAVITDEYKVPSKKFKVLGNTDRHSIKKNLIGALLEDSG | 33 |
| Streptococcus | ETAEATRLKRTARRRYTRRKNRICYLQEIFSNEMAKVDDSFFHRLEESFLVEEDKK | |
| pyogenes | HERHPIFGNIVDEVAYHEKYPTIYHLRKKLVDSTDKADLRLIYLALAHMIKFRGHFL | |
| Q99ZW2 Cas9 | IEGDLNPDNSDVDKLFIQLVQTYNQLFEENPINASGVDAKAILSARLSKSRRLENLIA | |
| with H840A | QLPGEKKNGLFGNLIALSLGLTPNFKSNEDLAEDAKLQLSKDTYDDDLDNLLAQIG | |
| | DQYADLFLAAKNLSDAILLSDILRVNTEITKAPLSASMIKRYDEHHQDLTLLKALVR | |
| | QQLPEKYKEIFFDQSKNGYAGYIDGGASQEEFYKFIKPILEKMDGTEELLVKLNRED | |
| | LLRKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHS | |
| | LLYEYFTVYNELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDILEDIVLTLTLF | |
| | EDREMIEERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKELGSQI | |
| | LKEHPVENTQLQNEKLYLYYLQNGRDMYVDQELDINRLSDYDVDAIVPQSFLKDD | |
| | SIDNKVLTRSDKNRGKSDNVPSEEVVKKMKNYWRQLLNAKLITQRKFDNLTKAER | |
| | GGLSELDKAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGDYKVYD | |
| | VRKMIAKSEQEIGKATAKYFFYSNIMNFFKTEITLANGEIRKRPLIETNGETGEIVWD | |
| | KGRDFATVRKVLSMPQVNIVKKTEVQTGGFSKESILPKRNSDKLIARKKDWDPKK | |
| | YGGFDSPTVAYSVLVVAKVEKGKSKKLKSVKELLGITIMERSSFEKNPIDFLEAKG | |
| | YKEVKKDLIIKLPKYSLFELENGRKRMLASAGELQKGNELALPSKYVNFLYLASHY | |
| | EKLKGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYET | |
| | RIDLSQLGGD | |
| Cas9 nickase | MDKKYSIGLDIGTNSVGWAVITDEYKVPSKKFKVLGNTDRHSIKKNLIGALLFDS | 34 |
| Streptococcus | ETAEATRLKRTARRRYTRRKNRICYLQEIFSNEMAKVDDSFFHRLEESFLVEEDKK | |
| pyogenes | HERHPIFGNIVDEVAYHEKYPTIYHLRKKLVDSTDKADLRLIYLALAHMIKFRGHFL | |
| Q99ZW2 Cas9 | HEGDLNPDNSDVDKLFIQLVQTYNQLFEENPINASGVDAKAILSARLSKSRRLENLIA | |
| with R863X, | QLPGEKKNGLFGNLIALSLGLTPNFKSNFDLAEDAKLQLSKDTYDDDLDNLLAQIG | |
| wherein X is any | DQYADLFLAAKNLSDAILLSDILRVNTEITKAPLSASMIKRYDEHHQDLTLLKALVR | |
| alternate amino | QQLPEKYKEIFFDQSKNGYAGYIDGGASQEEFYKFIKPILEKMDGTEELLVKLNRED | |
| acid | LLRKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHS | |
| | LLYEYFTVYNELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDILEDIVLTLTLF | |
| | EDREMIEERLKTYAHLEDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKELGSQI | |
| | LKEHPVENTQLQNEKLYLYYLQNGRDMYVDQELDINRLSDYDVDHIVPQSFLKDD | |
| | SIDNKVLTRSDKNXGKSDNVPSEEVVKKMKNYWRQLLNAKLITQRKFDNLTKAER | |
| | GGLSELDKAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGDYKVYD | |
| | VRKMIAKSEQEIGKATAKYFFYSNIMNFFKTEITLANGEIRKRPLIETNGETGEIVWD | |
| | KGRDFATVRKVLSMPQVNIVKKTEVQTGGFSKESILPKRNSDKLIARKKDWDPKK | |
| | YGGFDSPTVAYSVLVVAKVEKGKSKKLKSVKELLGITIMERSSFEKNPIDFLEAKG | |
| | YKEVKKDLIIKLPKYSLFELENGRKRMLASAGELQKGNELALPSKYVNFLYLASHY | |
| | EKLKGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYET | |
| | RIDLSQLGGD | |
| Cas9 nickase | MDKKYSIGLDIGTNSVGWAVITDEYKVPSKKFKVLGNTDRHSIKKNLIGALLEDSG | 35 |
| Streptococcus | ETAEATRLKRTARRRYTRRKNRICYLQEIFSNEMAKVDDSFFHRLEESFLVEEDKK | |
| pyogenes | HERHPIFGNIVDEVAYHEKYPTIYHLRKKLVDSTDKADLRLIYLALAHMIKFRGHFL | |
| Q99ZW2 Cas9 | JEGDLNPDNSDVDKLFIQLVQTYNQLFEENPINASGVDAKAILSARLSKSRRLENLIA | |
| with R863A | QLPGEKKNGLFGNLIALSLGLTPNFKSNEDLAEDAKLQLSKDTYDDDLDNLLAQIG | |
| | DQYADLFLAAKNLSDAILLSDILRVNTEITKAPLSASMIKRYDEHHQDLTLLKALVR | |
| | QQLPEKYKEIFFDQSKNGYAGYIDGGASQEEFYKFIKPILEKMDGTEELLVKLNRED | |
| | LLRKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHS | |
| | LLYEYFTVYNELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDILEDIVLTLTLF | |
| | EDREMIEERLKTYAHLEDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKELGSQI | |
| | LKEHPVENTQLQNEKLYLYYLQNGRDMYVDQELDINRLSDYDVDHIVPQSFLKDD | |
| | SIDNKVLTRSDKNAGKSDNVPSEEVVKKMKNYWRQLLNAKLITQRKFDNLTKAER | |
| | GGLSELDKAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGDYKVYD | |
| | VRKMIAKSEQEIGKATAKYFFYSNIMNFFKTEITLANGEIRKRPLIETNGETGEIVWD | |
| | KGRDFATVRKVLSMPQVNIVKKTEVQTGGFSKESILPKRNSDKLIARKKDWDPKK | |
| | YGGFDSPTVAYSVLVVAKVEKGKSKKLKSVKELLGITIMERSSFEKNPIDFLEAKG | |
| | YKEVKKDLIIKLPKYSLFELENGRKRMLASAGELQKGNELALPSKYVNFLYLASHY | |
| | EKLKGSPEDNEQKQLFVEQHKHYLDEIIEQISEFSKRVILADANLDKVLSAYNKHRD | |
| | KPIREQAENIIHLFILTNLGAPAAFKYFDTTIDRKRYTSTKEVLDATLIHQSITGLYET | |
| | RIDLSQLGGD | |

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto.

| Description | Sequence |
|---|---|
| Cas9 nickase | DKKYSIGLDIGTNSVGWAVITDEYKVPSKKFKVLGNTDRHSIKKNLIGALLFDSGETAEAT |
| (Met minus) | RLKRTARRRYTRRKNRICYLQEIFSNEMAKVDDSFFHRLEESFLVEEDKKHERHPIFGNIV |
| Streptococcus | DEVAYHEKYPTIYHLRKKLVDSTDKADLRLIYLALAHMIKFRGHFLIEGDLNPDNSDVDK |
| pyogenes | LFIQLVQTYNQLFEENPINASGVDAKAILSARLSKSRRLENLIAQLPGEKKNGLFGNLIALS |
| Q99ZW2 Cas9 | LGLTPNFKSNFDLAEDAKLQLSKDTYDDDLDNLLAQIGDQYADLFLAAKNLSDAILLSDI |
| with H840X, | LRVNTEITKAPLSASMIKRYDEHHQDLTLLKALVRQQLPEKYKEIFFDQSKNGYAGYIDG |
| wherein X is any | GASQEEFYKFIKPILEKMDGTEELLVKLNREDLLRKQRTFDNGSIPHQIHLGELHAILRRQE |
| alternate amino | DFYPFLKDNREKIEKILTFRIPYYVGPLARGNSRFAWMTRKSEETITPWNFEEVVDKGASA |
| acid | QSFIERMTNFDKNLPNEKVLPKHSLLYEYFTVYNELTKVKYVTEGMRKPAFLSGEQKKAI |
| | VDLLFKTNRKVTVKQLKEDYFKKIECFDSVEISGVEDRFNASLGTYHDLLKIIKDKDELDN |
| | EENEDILEDIVLTLTLFEDREMIEERLKTYAHLFDDKVMKQLKRRRYTGWGRLSRKLINGI |
| | RDKQSGKTILDFLKSDGFANRNEMQLIHDDSLTFKEDIQKAQVSGQGDSLHEHIANLAGS |
| | PAIKKGILQTVKVVDELVKVMGRHKPENIVIEMARENQTTQKGQKNSRERMKRIEEGIKE |
| | LGSQILKEHPVENTQLQNEKLYLYYLQNGRDMYVDQELDINRLSDYDVDXIVPQSFLKD |
| | DSIDNKVLTRSDKNRGKSDNVPSEEVVKKMKNYWRQLLNAKLITQRKFDNLTKAERGG |
| | LSELDKAGFIKRQLVETRQITKHVAQILDSRMNTKYDENDKLIREVKVITLKSKLVSDFRK |
| | DFQFYKVREINNYHHAHDAYLNAVVGTALIKKYPKLESEFVYGDYKVYDVRKMIAKSE |
| | QEIGKATAKYFFYSNIMNFFKTEITLANGEIRKRPLIETNGETGEIVWDKGRDFATVRKVL |
| | SMPQVNIVKKTEVQTGGFSKESILPKRNSDKLIARKKDWDPKKYGGEDSPTVAYSVLVVA |
| | KVEKGKSKKLKSVKELLGITIMERSSFEKNPIDFLEAKGYKEVKKDLIIKLPKYSLFELENG |
| | RKRMLASAGELQKGNELALPSKYVNFLYLASHYEKLKGSPEDNEQKQLFVEQHKHYLDE |
| | IIEQISEFSKRVILADANLDKVLSAYNKHRDKPIREQAENIIHLFILTNLGAPAAFKYFDTTI |
| | DRKRYTSTKEVLDATLIHQSITGLYETRIDLSQLGGD (SEQ ID NO: 36) |
| Cas9 nickase | DKKYSIGLDIGTNSVGWAVITDEYKVPSKKFKVLGNTDRHSIKKNLIGALLEDSGETAEAT |
| (Met minus) | RLKRTARRRYTRRKNRICYLQEIFSNEMAKVDDSFFHRLEESELVEEDKKHERHPIFGNIV |
| Streptococcus | DEVAYHEKYPTIYHLRKKLVDSTDKADLRLIYLALAHMIKFRGHFLIEGDLNPDNSDVDK |
| pyogenes | LFIQLVQTYNQLFEENPINASGVDAKAILSARLSKSRRLENLIAQLPGEKKNGLFGNLIALS |
| Q99ZW2 Cas9 | LGLTPNFKSNFDLAEDAKLQLSKDTYDDDLDNLLAQIGDQYADLFLAAKNLSDAILLSDI |
| with H840A | LRVNTEITKAPLSASMIKRYDEHHQDLTLLKALVRQQLPEKYKEIFFDQSKNGYAGYIDG |
| | GASQEEFYKFIKPILEKMDGTEELLVKLNREDLLRKQRTFDNGSIPHQIHLGELHAILRRQE |
| | DFYPFLKDNREKIEKILTFRIPYYVGPLARGNSRFAWMTRKSEETITPWNFEEVVDKGASA |
| | QSFIERMTNFDKNLPNEKVLPKHSLLYEYFTVYNELTKVKYVTEGMRKPAFLSGEQKKAI |
| | VDLLFKTNRKVTVKQLKEDYFKKIECFDSVEISGVEDRENASLGTYHDLLKIIKDKDFLDN |
| | EENEDILEDIVLTLTLFEDREMIEERLKTYAHLEDDKVMKQLKRRRYTGWGRLSRKLINGI |
| | RDKQSGKTILDFLKSDGFANRNFMQLIHDDSLTFKEDIQKAQVSGQGDSLHEHIANLAGS |
| | PAIKKGILQTVKVVDELVKVMGRHKPENIVIEMARENQTTQKGQKNSRERMKRIEEGIKE |
| | LGSQILKEHPVENTQLQNEKLYLYYLQNGRDMYVDQELDINRLSDYDVDAIVPQSFLKD |
| | DSIDNKVLTRSDKNRGKSDNVPSEEVVKKMKNYWRQLLNAKLITQRKFDNLTKAERGG |
| | LSELDKAGFIKRQLVETRQITKHVAQILDSRMNTKYDENDKLIREVKVITLKSKLVSDFRK |
| | DFQFYKVREINNYHHAHDAYLNAVVGTALIKKYPKLESEFVYGDYKVYDVRKMIAKSE |
| | QEIGKATAKYFFYSNIMNFFKTEITLANGEIRKRPLIETNGETGEIVWDKGRDFATVRKVL |
| | SMPQVNIVKKTEVQTGGFSKESILPKRNSDKLIARKKDWDPKKYGGFDSPTVAYSVLVVA |
| | KVEKGKSKKLKSVKELLGITIMERSSFEKNPIDFLEAKGYKEVKKDLIIKLPKYSLFELENG |
| | RKRMLASAGELQKGNELALPSKYVNFLYLASHYEKLKGSPEDNEQKQLFVEQHKHYLDE |
| | HIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD (SEQ ID NO: 37) |
| Cas9 nickase | DKKYSIGLDIGTNSVGWAVITDEYKVPSKKFKVLGNTDRHSIKKNLIGALLEDSGETAEAT |
| (Met minus) | RLKRTARRRYTRRKNRICYLQEIFSNEMAKVDDSFFHRLEESFLVEEDKKHERHPIFGNIV |
| Streptococcus | DEVAYHEKYPTIYHLRKKLVDSTDKADLRLIYLALAHMIKFRGHFLIEGDLNPDNSDVDK |
| pyogenes | LFIQLVQTYNQLFEENPINASGVDAKAILSARLSKSRRLENLIAQLPGEKKNGLFGNLIALS |
| Q99ZW2 Cas9 | LGLTPNFKSNEDLAEDAKLQLSKDTYDDDLDNLLAQIGDQYADLFLAAKNLSDAILLSDI |
| with R863X, | LRVNTEITKAPLSASMIKRYDEHHQDLTLLKALVRQQLPEKYKEIFFDQSKNGYAGYIDG |
| wherein X is any | GASQEEFYKFIKPILEKMDGTEELLVKLNREDLLRKQRTFDNGSIPHQIHLGELHAILRRQE |
| alternate amino | DFYPFLKDNREKIEKILTFRIPYYVGPLARGNSRFAWMTRKSEETITPWNFEEVVDKGASA |
| acid | QSFIERMTNFDKNLPNEKVLPKHSLLYEYFTVYNELTKVKYVTEGMRKPAFLSGEQKKAI |
| | VDLLFKTNRKVTVKQLKEDYFKKIECFDSVEISGVEDRENASLGTYHDLLKIIKDKDFLDN |
| | EENEDILEDIVLTLTLFEDREMIEERLKTYAHLFDDKVMKQLKRRRYTGWGRLSRKLINGI |
| | RDKQSGKTILDFLKSDGFANRNFMQLIHDDSLTFKEDIQKAQVSGQGDSLHEHIANLAGS |
| | PAIKKGILQTVKVVDELVKVMGRHKPENIVIEMARENQTTQKGQKNSRERMKRIEEGIKE |
| | LGSQILKEHPVENTQLQNEKLYLYYLQNGRDMYVDQELDINRLSDYDVDHIVPQSFLKD |
| | DSIDNKVLTRSDKNXGKSDNVPSEEVVKKMKNYWRQLLNAKLITQRKFDNLTKAERGG |
| | LSELDKAGFIKRQLVETRQITKHVAQILDSRMNTKYDENDKLIREVKVITLKSKLVSDFRK |
| | DFQFYKVREINNYHHAHDAYLNAVVGTALIKKYPKLESEFVYGDYKVYDVRKMIAKSE |
| | QEIGKATAKYFFYSNIMNFFKTEITLANGEIRKRPLIETNGETGEIVWDKGRDFATVRKVL |
| | SMPQVNIVKKTEVQTGGFSKESILPKRNSDKLIARKKDWDPKKYGGFDSPTVAYSVLVVA |
| | KVEKGKSKKLKSVKELLGITIMERSSFEKNPIDFLEAKGYKEVKKDLIIKLPKYSLFELENG |
| | RKRMLASAGELQKGNELALPSKYVNFLYLASHYEKLKGSPEDNEQKQLFVEQHKHYLDE |
| | HIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD(SEQ ID NO: 38) |
| Cas9 nickase | DKKYSIGLDIGTNSVGWAVITDEYKVPSKKFKVLGNTDRHSIKKNLIGALLEDSGETAEAT |
| (Met minus) | RLKRTARRRYTRRKNRICYLQEIFSNEMAKVDDSFFHRLEESFLVEEDKKHERHPIFGNIV |
| Streptococcus | DEVAYHEKYPTIYHLRKKLVDSTDKADLRLIYLALAHMIKFRGHFLIEGDLNPDNSDVDK |
| pyogenes | LFIQLVQTYNQLFEENPINASGVDAKAILSARLSKSRRLENLIAQLPGEKKNGLFGNLIALS |
| Q99ZW2 Cas9 | LGLTPNFKSNFDLAEDAKLQLSKDTYDDDLDNLLAQIGDQYADLFLAAKNLSDAILLSDI |
| with R863A | LRVNTEITKAPLSASMIKRYDEHHQDLTLLKALVRQQLPEKYKEIFFDQSKNGYAGYIDG |
| | GASQEEFYKFIKPILEKMDGTEELLVKLNREDLLRKQRTFDNGSIPHQIHLGELHAILRRQE |
| | DFYPFLKDNREKIEKILTFRIPYYVGPLARGNSRFAWMTRKSEETITPWNFEEVVDKGASA |
| | QSFIERMTNFDKNLPNEKVLPKHSLLYEYFTVYNELTKVKYVTEGMRKPAFLSGEQKKAI |
| | VDLLFKTNRKVTVKQLKEDYFKKIECFDSVEISGVEDRENASLGTYHDLLKIIKDKDFLDN |
| | EENEDILEDIVLTLTLFEDREMIEERLKTYAHLEDDKVMKQLKRRRYTGWGRLSRKLINGI |
| | RDKQSGKTILDFLKSDGFANRNFMQLIHDDSLTFKEDIQKAQVSGQGDSLHEHIANLAGS |
| | PAIKKGILQTVKVVDELVKVMGRHKPENIVIEMARENQTTQKGQKNSRERMKRIEEGIKE |
| | LGSQILKEHPVENTQLQNEKLYLYYLQNGRDMYVDQELDINRLSDYDVDHIVPQSFLKD |
| | DSIDNKVLTRSDKNAGKSDNVPSEEVVKKMKNYWRQLLNAKLITQRKFDNLTKAERGG |
| | LSELDKAGFIKRQLVETRQITKHVAQILDSRMNTKYDENDKLIREVKVITLKSKLVSDFRK |
| | DFQFYKVREINNYHHAHDAYLNAVVGTALIKKYPKLESEFVYGDYKVYDVRKMIAKSE |
| | QEIGKATAKYFFYSNIMNFFKTEITLANGEIRKRPLIETNGETGEIVWDKGRDFATVRKVL |
| | SMPQVNIVKKTEVQTGGFSKESILPKRNSDKLIARKKDWDPKKYGGFDSPTVAYSVLVVA |
| | KVEKGKSKKLKSVKELLGITIMERSSFEKNPIDFLEAKGYKEVKKDLIIKLPKYSLFELENG |
| | RKRMLASAGELQKGNELALPSKYVNFLYLASHYEKLKGSPEDNEQKQLFVEQHKHYLDE |
| | IIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD (SEQ ID NO: 39) |

### E. Uther Cas9 Variants

Besides dead Cas9 and Cas9 nickase variants, the Cas9 proteins used herein may also include other “Cas9 variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42.43, 44, 45, 46, 47, 48, 49, 50 or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SEQ ID NO: 2).

In some embodiments, the disclosure also may utilize Cas9 fragments that retain their functionality and that are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In various embodiments, the prime editors utilized in the methods and compositions disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

### F. Small-Sized Cas9 Variants

In some embodiments, the prime editors utilized in the methods and compositions contemplated herein can include a Cas9 protein that is of smaller molecular weight than the canonical SpCas9 sequence. In some embodiments, the smaller-sized Cas9 variants may facilitate delivery to cells, e.g., by an expression vector, nanoparticle, or other means of delivery. In certain embodiments, the smaller-sized Cas9 variants can include enzymes categorized as type II enzymes of the Class 2 CRISPR-Cas systems. In some embodiments, the smaller-sized Cas9 variants can include enzymes categorized as type V enzymes of the Class 2 CRISPR-Cas systems. In other embodiments, the smaller-sized Cas9 variants can include enzymes categorized as type VI enzymes of the Class 2 CRISPR-Cas systems.

The canonical SpCas9 protein is 1368 amino acids in length and has a predicted molecular weight of 158 kilodaltons. The term “small-sized Cas9 variant”, as used herein, refers to any Cas9 variant—naturally occurring, engineered, or otherwise—that is less than at least 1300 amino acids, or at least less than 1290 amino acids, or than less than 1280 amino acids, or less than 1270 amino acid, or less than 1260 amino acid, or less than 1250 amino acids, or less than 1240 amino acids, or less than 1230 amino acids, or less than 1220 amino acids, or less than 1210 amino acids, or less than 1200 amino acids, or less than 1190 amino acids, or less than 1180 amino acids, or less than 1170 amino acids, or less than 1160 amino acids, or less than 1150 amino acids, or less than 1 140 amino acids, or less than 1130 amino acids, or less than 1120 amino acids, or less than 1110 amino acids, or less than 1100 amino acids, or less than 1050 amino acids, or less than 1000 amino acids, or less than 950 amino acids, or less than 900 amino acids, or less than 850 amino acids, or less than 800 amino acids, or less than 750 amino acids, or less than 700 amino acids, or less than 650 amino acids, or less than 600 amino acids, or less than 550 amino acids, or less than 500 amino acids, but at least larger than about 400 amino acids and retaining the required functions of the Cas9 protein. The Cas9 variants can include those categorized as type II, type V, or type VI enzymes of the Class 2 CRISPR-Cas system.

In various embodiments, the prime editors utilized in the methods and compositions disclosed herein may comprise one of the small-sized Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference small-sized Cas9 protein.

| | | SEQ |
|---|---|---|
| Description | Sequence | ID NO: |
| SaCas9 | MGKRNYILGLDIGITSVGYGIIDYETRDVIDAGVRLFKEANVENNEGRRSKRGARR | 10 |
| Staphylococcus | LKRRRRHRIQRVKKLLFDYNLLTDHSELSGINPYEARVKGLSQKLSEEEFSAALLHL | |
| aureus | AKRRGVHNVNEVEEDTGNELSTKEQISRNSKALEEKYVAELQLERLKKDGEVRGSI | |
| 1053 AA | NRFKTSDYVKEAKQLLKVQKAYHQLDQSFIDTYIDLLETRRTYYEGPGEGSPFGW | |
| 123 kDa | KDIKEWYEMLMGHCTYFPEELRSVKYAYNADLYNALNDLNNLVITRDENEKLEY | |
| | YEKFQIIENVFKQKKKPTLKQIAKEILVNEEDIKGYRVTSTGKPEFTNLKVYHDIKDI | |
| | TARKEIIENAELLDQIAKILTIYQSSEDIQEELTNLNSELTQEEIEQISNLKGYTGTHNL | |
| | SLKAINLILDELWHTNDNQIAIFNRLKLVPKKVDLSQQKEIPTTLVDDFILSPVVKRS | |
| | FIQSIKVINAIIKKYGLPNDIIIELAREKNSKDAQKMINEMQKRNRQTNERIEEIIRTTG | |
| | KENAKYLIEKIKLHDMQEGKCLYSLEAIPLEDLLNNPFNYEVDHIIPRSVSFDNSENN | |
| | KVLVKQEENSKKGNRTPFQYLSSSDSKISYETFKKHILNLAKGKGRISKTKKEYLLE | |
| | ERDINRFSVQKDFINRNLVDTRYATRGLMNLLRSYFRVNNLDVKVKSINGGFTSEL | |
| | RRKWKFKKERNKGYKHHAEDALIIANADFIFKEWKKLDKAKKVMENQMFEEKQA | |
| | ESMPEIETEQEYKEIFITPHQIKHIKDFKDYKYSHRVDKKPNRKLINDTLYSTRKDDK | |
| | GNTLIVNNLNGLYDKDNDKLKKLINKSPEKLLMYHHDPQTYQKLKLIMEQYGDEK | |
| | NPLYKYYEETGNYLTKYSKKDNGPVIKKIKYYGNKLNAHLDITDDYPNSRNKVVK | |
| | LSLKPYRFDVYLDNGVYKFVTVKNLDVIKKENYYEVNSKCYEEAKKLKKISNQAE | |
| | FIASFYKNDLIKINGELYRVIGVNNDLLNRIEVNMIDITYREYLENMNDKRPPHIIKTI | |
| | ASKTQSIKKYSTDILGNLYEVKSKKHPQIIKK | |
| NmeCas9 | MAAFKPNSINYILGLDIGIASVGWAMVEIDEEENPIRLIDLGVRVFERAEVPKTGDSL | 40 |
| N. meningitidis | AMARRLARSVRRLTRRRAHRLLRTRRLLKREGVLQAANFDENGLIKSLPNTPWQL | |
| 1083 AA | RAAALDRKLTPLEWSAVLLHLIKHRGYLSQRKNEGETADKELGALLKGVAGNAH | |
| 124.5 kDa | ALQTGDFRTPAELALNKFEKESGHIRNQRSDYSHTFSRKDLQAELILLFEKQKEFGN | |
| | PHVSGGLKEGIETLLMTQRPALSGDAVQKMLGHCTFEPAEPKAAKNTYTAERFIW | |
| | LTKLNNLRILEQGSERPLTDTERATLMDEPYRKSKLTYAQARKLLGLEDTAFFKGL | |
| | RYGKDNAEASTLMEMKAYHAISRALEKEGLKDKKSPLNLSPELQDEIGTAFSLEKT | |
| | DEDITGRLKDRIQPEILEALLKHISFDKFVQISLKALRRIVPLMEQGKRYDEACABIY | |
| | GDHYGKKNTEEKIYLPPIPADEIRNPVVLRALSQARKVINGVVRRYGSPARIHIETA | |
| | REVGKSFKDRKEIEKRQEENRKDREKAAAKFREYFPNFVGEPKSKDILKLRLYEQQ | |
| | HGKCLYSGKEINLGRLNEKGYVEIDAALPFSRTWDDSFNNKVLVLGSENQNKGNQ | |
| | TPYEYFNGKDNSREWQEFKARVETSRFPRSKKQRILLQKFDEDGFKERNLNDTRYV | |
| | NRFLCQFVADRMRLTGKGKKRVFASNGQITNLLRGFWGLRKVRAENDRHHALDA | |
| | VVVACSTVAMQQKITRFVRYKEMNAFDGKTIDKETGEVLHQKTHFPQPWEFFAQE | |
| | VMIRVFGKPDGKPEFEEADTLEKLRTLLAEKLSSRPEAVHEYVTPLFVSRAPNRKM | |
| | SGQGHMETVKSAKRLDEGVSVLRVPLTQLKLKDLEKMVNREREPKLYEALKARL | |
| | EAHKDDPAKAFAEPFYKYDKAGNRTQQVKAVRVEQVQKTGVWVRNHNGIADNA | |
| | TMVRVDVFEKGDKYYLVPIYSWQVAKGILPDRAVVQGKDEEDWQLIDDSENFKFS | |
| | LHPNDLVEVITKKARMFGYFASCHRGTGNINIRIHDLDHKIGKNGILEGIGVKTALS | |
| | FQKYQIDELGKEIRPCRLKKRPPVR | |
| CjCas9 | MARILAFDIGISSIGWAFSENDELKDCGVRIFTKVENPKTGESLALPRRLARSARKRL | 41 |
| C. jejuni | ARRKARLNHLKHLIANEFKLNYEDYQSFDESLAKAYKGSLISPYELRFRALNELLS | |
| 984 AA | KQDFARVILHIAKRRGYDDIKNSDDKEKGAILKAIKQNEEKLANYQSVGEYLYKEY | |
| 114.9 kDa | FQKFKENSKEFTNVRNKKESYERCIAQSFLKDELKLIFKKQREFGFSFSKKFEEEVLS | |
| | VAFYKRALKDESHLVGNCSFFTDEKRAPKNSPLAFMFVALTRIINLLNNLKNTEGIL | |
| | YTKDDLNALLNEVLKNGTLTYKQTKKLLGLSDDYEFKGEKGTYFIEFKKYKEFIKA | |
| | LGEHNLSQDDLNEIAKDITLIKDEIKLKKALAKYDLNQNQIDSLSKLEFKDHLNISFK | |
| | ALKLVTPLMLEGKKYDEACNELNLKVAINEDKKDFLPAFNETYYKDEVTNPVVLR | |
| | AIKEYRKVLNALLKKYGKVHKINIELAREVGKNHSQRAKIEKEQNENYKAKKDAE | |
| | LECEKLGLKINSKNILKLRLFKEQKEFCAYSGEKIKISDLQDEKMLEIDHIYPYSRSF | |
| | DDSYMNKVLVFTKQNQEKLNQTPFEAFGNDSAKWQKIEVLAKNLPTKKQKRILDK | |
| | NYKDKEQKNFKDRNLNDTRYIARLVLNYTKDYLDFLPLSDDENTKLNDTQKGSKV | |
| | HVEAKSGMLTSALRHTWGFSAKDRNNHLHHAIDAVIIAYANNSIVKAFSDFKKEQ | |
| | ESNSAELYAKKISELDYKNKRKFFEPFSGFRQKVLDKIDEIFVSKPERKKPSGALHEE | |
| | TFRKEEEFYQSYGGKEGVLKALELGKIRKVNGKIVKNGDMFRVDIFKHKKTNKFY | |
| | AVPIYTMDFALKVLPNKAVARSKKGEIKDWILMDENYEFCFSLYKDSLILIQTKDM | |
| | QEPEFVYYNAFTSSTVSLIVSKHDNKFETLSKNQKILFKNANEKEVIAKSIGIQNLKV | |
| | FEKYIVSALGEVTKAEFRQREDFKK | |
| GeoCas9 | MRYKIGLDIGITSVGWAVMNLDIPRIEDLGVRIFDRAENPQTGESLALPRRLARSAR | 42 |
| G. | RRLRRRKHRLERIRRLVIREGILTKEELDKLFEEKHEIDVWQLRVEALDRKLNNDEL | |
| stearothermophilus | ARVLLHLAKRRGFKSNRKSERSNKENSTMLKHIEENRAILSSYRTVGEMIVKDPKF | |
| 1087 AA | ALHKRNKGENYTNTIARDDLEREIRLIFSKQREFGNMSCTEEFENEYITIWASQRPV | |
| 127 kDa | ASKDDIEKKVGFCTFEPKEKRAPKATYTFQSFIAWEHINKLRLISPSGARGLTDEER | |
| | RLLYEQAFQKNKITYHDIRTLLHLPDDTYFKGIVYDRGESRKQNENIRFLELDAYH | |
| | QIRKAVDKVYGKGKSSSFLPIDFDTFGYALTLFKDDADIHSYLRNEYEQNGKRMPN | |
| | LANKVYDNELIEELLNLSFTKFGHLSLKALRSILPYMEQGEVYSSACERAGYTFTGP | |
| | KKKQKTMLLPNIPPIANPVVMRALTQARKVVNAIIKKYGSPVSIHIELARDLSQTFD | |
| | ERRKTKKEQDENRKKNETAIRQLMEYGLTLNPTGHDIVKFKLWSEQNGRCAYSLQ | |
| | PIEIERLLEPGYVEVDHVIPYSRSLDDSYTNKVLVLTRENREKGNRIPAEYLGVGTE | |
| | RWQQFETFVLTNKQFSKKKRDRLLRLHYDENEETEFKNRNLNDTRYISRFFANFIR | |
| | EHLKFAESDDKQKVYTVNGRVTAHLRSRWEFNKNREESDLHHAVDAVIVACTTPS | |
| | DIAKVTAFYQRREQNKELAKKTEPHFPQPWPHFADELRARLSKHPKESIKALNLGN | |
| | YDDQKLESLQPVFVSRMPKRSVTGAAHQETLRRYVGIDERSGKIQTVVKTKLSEIK | |
| | LDASGHFPMYGKESDPRTYEAIRQRLLEHNNDPKKAFQEPLYKPKKNGEPGPVIRT | |
| | VKIIDTKNQVIPLNDGKTVAYNSNIVRVDVFEKDGKYYCVPVYTMDIMKGILPNKÅ | |
| | IEPNKPYSEWKEMTEDYTFRFSLYPNDLIRIELPREKTVKTAAGEEINVKDVFVYYK | |
| | TIDSANGGLELISHDHRFSLRGVGSRTLKRFEKYQVDVLGNIYKVRGEKRVGLASS | |
| | AHSKPGKTIRPLQSTRD | |
| LbaCas12a | MSKLEKFTNCYSLSKTLRFKAIPVGKTQENIDNKRLLVEDEKRAEDYKGVKKLLDR | 43 |
| L. bacterium | YYLSFINDVLHSIKLKNLNNYISLFRKKTRTEKENKELENLEINLRKEIAKAFKGNEG | |
| 1228 AA | YKSLFKKDIIETILPEFLDDKDEIALVNSENGFTTAFTGFFDNRENMFSEEAKSTSIAF | |
| 143,9 kDa | RCINENLTRYISNMDIFEKVDAIFDKHEVQEIKEKILNSDYDVEDFFEGEFFNFVLTQ | |
| | EGIDVYNAHIGGFVTESGEKIKGLNEYINLYNQKTKQKLPKFKPLYKQVLSDRESLS | |
| | FYGEGYTSDEEVLEVFRNTLNKNSEIFSSIKKLEKLFKNFDEYSSAGIFVKNGPAISTI | |
| | SKDIFGEWNVIRDKWNAEYDDIHLKKKAVVTEKYEDDRRKSFKKIGSFSLEQLQEY | |
| | ADADLSVVEKLKEIIIQKVDEIYKVYGSSEKLFDADFVLEKSLKKNDAVVAIMKDL | |
| | LDSVKSFENYIKAFFGEGKETNRDESFYGDFVLAYDILLKVDHIYDAIRNYVTQKPY | |
| | SKDKFKLYFQNPQFMGGWDKDKETDYRATILRYGSKYYLAIMDKKYAKCLQKID | |
| | KDDVNGNYEKINYKLLPGPNKMLPKVFFSKKWMAYYNPSEDIQKIYKNGTFKKG | |
| | DMENLNDCHKLIDFFKDSISRYPKWSNAYDFNFSETEKYKDIAGFYREVEEQGYKV | |
| | SFESASKKEVDKLVEEGKLYMFQIYNKDFSDKSHGTPNLHTMYFKLLFDENNHGQI | |
| | RLSGGAELFMRRASLKKEELVVHPANSPIANKNPDNPKKTTTLSYDVYKDKRFSED | |
| | QYELHIPIAINKCPKNIFKINTEVRVLLKHDDNPYVIGIDRGERNLLYIVVVDGKGNI | |
| | VEQYSLNEIINNENGIRIKTDYHSLLDKKEKERFEARQNWTSIENIKELKAGYISQVV | |
| | HKICELVEKYDAVIALEDLNSGFKNSRVKVEKQVYQKFEKMLIDKLNYMVDKKSN | |
| | PCATGGALKGYQITNKFESFKSMSTQNGFIFYIPAWLTSKIDPSTGFVNLLKTKYTSI | |
| | ADSKKFISSFDRIMYVPEEDLFEFALDYKNFSRTDADYIKKWKLYSYGNRIRIFRNP | |
| | KKNNVFDWEEVCLTSAYKELFNKYGINYQQGDIRALLCEQSDKAFYSSFMALMSL | |
| | MLQMRNSITGRTDVDFLISPVKNSDGIFYDSRNYEAQENAILPKNADANGAYNIAR | |
| | KVLWAIGQFKKAEDEKLDKVKIAISNKEWLEYAQTSVKH | |
| BhCas12b | MATRSFILKIEPNEEVKKGLWKTHEVLNHGIAYYMNILKLIRQEAIYEHHEQDPKNP | 44 |
| B. hisashii | KKVSKAEIQAELWDFVLKMQKCNSFTHEVDKDEVFNILRELYEELVPSSVEKKGE | |
| 1108 AA | ANQLSNKFLYPLVDPNSQSGKGTASSGRKPRWYNIKIAGDPSWEEEKKKWEEDK | |
| 130.4 kDa | KKDPLAKILGKLAEYGLIPLFIPYTDSNEPIVKEIKWMEKSRNQSVRRLDKDMFIQA | |
| | LERFLSWESWNLKVKEEYEKVEKEYKTLEERIKEDIQALKALEQYEKERQEQLLRD | |
| | TLNTNEYRLSKRGLRGWREIIQKWLKMDENEPSEKYLEVFKDYQRKHPREAGDYS | |
| | VYEFLSKKENHFIWRNHPEYPYLYATFCEIDKKKKDAKQQATFTLADPINHPLWVR | |
| | FEERSGSNLNKYRILTEQLHTEKLKKKLTVQLDRLIYPTESGGWEEKGKVDIVLLPS | |
| | RQFYNQIFLDIEEKGKHAFTYKDESIKFPLKGTLGGARVQFDRDHLRRYPHKVESG | |
| | NVGRIYFNMTVNIEPTESPVSKSLKIHRDDFPKVVNFKPKELTEWIKDSKGKKLKSG | |
| | IESLEIGLRVMSIDLGQRQAAAASIFEVVDQKPDIEGKLFFPIKGTELYAVHRASENI | |
| | KLPGETLVKSREVLRKAREDNLKLMNQKLNFLRNVLHFQQFEDITEREKRVTKWIS | |
| | RQENSDVPLVYQDELIQIRELMYKPYKDWVAFLKQLHKRLEVEIGKEVKHWRKSL | |
| | SDGRKGLYGISLKNIDEIDRTRKFLLRWSLRPTEPGEVRRLEPGQRFAIDQLNHLNA | |
| | LKEDRLKKMANTIIMHALGYCYDVRKKKWQAKNPACQHILFEDLSNYNPYEERSR | |
| | FENSKLMKWSRREIPRQVALQGEIYGLQVGEVGAQFSSRFHAKTGSPGIRCSVVTK | |
| | EKLQDNRFFKNLQREGRLTLDKIAVLKEGDLYPDKGGEKFISLSKDRKCVTTHADI | |
| | NAAQNLQKRFWTRTHGFYKVYCKAYQVDGQTVYIPESKDQKQKITEEFGEGYFIL | |
| | KDGVYEWVNAGKLKIKKGSSKQSSSELVDSDILKDSFDLASELKGEKLMLYRDPS | |
| | GNVFPSDKWMAAGVFFGKLERILISKLTNQYSISTIEDDSSKQSM | |

### G. Cas9 Equivalents

In some embodiments, the prime editors utilized in the methods and compositions described herein can include any Cas9 equivalent. As used herein, the term “Cas9 equivalent” is a broad term that encompasses any napDNAbp protein that serves the same function as Cas9 in the prime editors despite that its amino acid primary sequence and/or its three-dimensional structure may be different and/or unrelated from an evolutionary standpoint. Thus, while Cas9 equivalents include any Cas9 ortholog, homolog, mutant, or variant described or embraced herein that are evolutionarily related, the Cas9 equivalents also embrace proteins that may have evolved through convergent evolution processes to have the same or similar function as Cas9, but that do not necessarily have any similarity with regard to amino acid sequence and/or three-dimensional structure. The prime editors utilized in the methods and compositions described here embrace any Cas9 equivalent that would provide the same or similar function as Cas9 despite that the Cas9 equivalent may be based on a protein that arose through convergent evolution. For instance, if Cas9 refers to a type II enzyme of the CRISPR-Cas system, a Cas9 equivalent can refer to a type V or type VI enzyme of the CRISPR-Cas system.

For example, Cas12e (CasX) is a Cas9 equivalent that reportedly has the same function as Cas9 but which evolved through convergent evolution. Thus, the Cas12e (CasX) protein described in Liu et al., “CasX enzymes comprises a distinct family of RNA-guided genome editors,” Nature, 2019, Vol. 566: 218-223, is contemplated to be used with the prime editors utilized in the methods and compositions described herein. In addition, any variant or modification of Cas12e (CasX) is conceivable and within the scope of the present disclosure.

Cas9 is a bacterial enzyme that evolved in a wide variety of species. However, the Cas9 equivalents contemplated herein may also be obtained from archaea, which constitute a domain and kingdom of single-celled prokaryotic microbes different from bacteria. In some embodiments, Cas9 equivalents may refer to Cas12e (CasX) or Cas12d (CasY), which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-Cas12e and CRISPR-Cas12d, which are among the most compact systems yet discovered. In some embodiments, Cas9 refers to Cas12e, or a variant of Cas12e. In some embodiments, Cas9 refers to a Cas12d, or a variant of Cas12d. It should be appreciated that other RNA-guided DNA binding proteins may be used as a nucleic acid programmable DNA binding protein (napDNAbp) and are within the scope of this disclosure. Also see Liu et al., “CasX enzymes comprises a distinct family of RNA-guided genome editors,” Nature, 2019, Vol. 566: 218-223. Any of these Cas9 equivalents are contemplated.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12e (CasX) or Cas12d (CasY) protein. In some embodiments, the napDNAbp is a naturally-occurring Cas12e (CasX) or Cas12d (CasY) protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the nucleic acid programmable DNA binding proteins include, without limitation, Cas9 (e.g., dCas9 and nCas9), Cas12e (CasX), Cas12d (CasY), Cas12a (Cpf1), Cas12b1 (C2c1), Cas13a (C2c2), Cas12c (C2c3), Argonaute, and Cas12b1. One example of a nucleic acid programmable DNA-binding protein that has different PAM specificity than Cas9 is Clustered Regularly Interspaced Short Palindromic Repeats from Prevotella and Francisella 1 (i.e., Cas12a (Cpf1)). Similar to Cas9, Cas12a (Cpf1) is also a Class 2 CRISPR effector, but it is a member of type V subgroup of enzymes, rather than the type II subgroup. It has been shown that Cas12a (Cpf1) mediates robust DNA interference with features distinct from Cas9, Cas12a (Cpf1) is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference.

In still other embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3. Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 2).

In various other embodiments, the napDNAbp can be any of the following proteins: a Cas9, a Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12g. a Cas12h, a Cas12i, a Cas13b, a Cas13c, a Cas 13d, a Cas 14, a Csn2, an xCas9, an SpCas9-NG, a circularly permuted Cas9, or an Argonaute (Ago) domain, or a variant thereof.

Exemplary Cas9 equivalent protein sequences can include the following:

| Description | Sequence |
|---|---|
| AsCas12a | HAALDQPLPTTLKKQEEKEILKSQLDSLLGLYHLLDWFAVDESNEVDPEFSARLTGIKLEME |
| (previously | MTQFEGFTNLYQVSKTLRFELIPQGKTLKHIQEQGFIEEDKARNDHYKELKPIIDRIYKTYAD |
| known as | QCLQLVQLDWENLSAAIDSYRKEKTEETRNALIEEQATYRNAIHDYFIGRTDNLTDAINKRH |
| Cpf1) | AEIYKGLFKAELFNGKVLKQLGTVTTTEHENALLRSFDKFTTYFSGFYENRKNVFSAEDIST |
| Acidaminococcus | AIPHRIVQDNFPKFKENCHIFTRLITAVPSLREHFENVKKAIGIFVSTSIEEVFSFPFYNQLLTQ |
| sp. (strain | TQIDLYNQLLGGISREAGTEKIKGLNEVLNLAIQKNDETAHIIASLPHRFIPLFKQILSDRNTLS |
| BV316) UniProtKB | FILEEFKSDEEVIQSFCKYKTLLRNENVLETAEALFNELNSIDLTHIFISHKKLETISSALCDHW |
| U2UMQ6 | DTLRNALYERRISELTGKITKSAKEKVQRSLKHEDINLQEIISAAGKELSEAFKQKTSEILSHA |
| | PSLSFYNKARNYATKKPYSVEKFKLNFQMPTLASGWDVNKEKNNGAILFVKNGLYYLGIM |
| | PKQKGRYKALSFEPTEKTSEGFDKMYYDYFPDAAKMIPKCSTQLKAVTAHFQTHTTPILLS |
| | NNFIEPLEITKEIYDLNNPEKEPKKFQTAYAKKTGDQKGYREALCKWIDFTRDFLSKYTKTT |
| | SIDLSSLRPSSQYKDLGEYYAELNPLLYHISFQRIAEKEIMDAVETGKLYLFQIYNKDFAKGH |
| | HGKPNLHTLYWTGLFSPENLAKTSIKLNGQAELFYRPKSRMKRMAHRLGEKMLNKKLKDQ |
| | KTPIPDTLYQELYDYVNHRLSHDLSDEARALLPNVITKEVSHEIIKDRRFTSDKFFFHVPITLN |
| | YQAANSPSKFNQRVNAYLKEHPETPIIGIDRGERNLIYITVIDSTGKILEQRSLNTIQQFDYQK |
| | KLDNREKERVAARQAWSVVGTIKDLKQGYLSQVIHEIVDLMIHYQAVVVLENLNFGFKSK |
| | RTGIAEKAVYQQFEKMLIDKLNCLVLKDYPAEKVGGVLNPYQLTDQFTSFAKMGTQSGFLF |
| | YVPAPYTSKIDPLTGFVDPFVWKTIKNHESRKHFLEGFDFLHYDVKTGDFILHFKMNRNLSF |
| | QRGLPGFMPAWDIVFEKNETQFDAKGTPFIAGKRIVPVIENHRFTGRYRDLYPANELIALLEE |
| | KGIVFRDGSNILPKLLENDDSHAIDTMVALIRSVLQMRNSNAATGEDYINSPVRDLNGVCFD |
| | SRFQNPEWPMDADANGAYHIALKGQLLLNHLKESKDLKLQNGISNQDWLAYIQELRN |
| | (SEQ ID NO: 45) |
| AsCas12a | MTQFEGFTNLYQVSKTLRFELIPQGKTLKHIQEQGFIEEDKARNDHYKELKPIIDRIYKTYAD |
| nickase (e.g., | QCLQLVQLDWENLSAAIDSYRKEKTEETRNALIEEQATYRNAIHDYFIGRTDNLTDAINKRH |
| R1226A) | AEIYKGLFKAELFNGKVLKQLGTVTTTEHENALLRSFDKFTTYFSGFYENRKNVFSAEDIST |
| | AIPHRIVQDNFPKFKENCHIFTRLITAVPSLREHFENVKKAIGIFVSTSIEEVFSFPFYNQLLTQ |
| | TQIDLYNQLLGGISREAGTEKIKGLNEVLNLAIQKNDETAHIIASLPHRFIPLFKQILSDRNTLS |
| | FILEEFKSDEEVIQSFCKYKTLLRNENVLETAEALFNELNSIDLTHIFISHKKLETISSALCDHW |
| | DTLRNALYERRISELTGKITKSAKEKVQRSLKHEDINLQEISAAGKELSEAFKQKTSEILSHA |
| | HAALDQPLPTTLKKQEEKEILKSQLDSLLGLYHLLDWFAVDESNEVDPEFSARLTGIKLEME |
| | PSLSFYNKARNYATKKPYSVEKFKLNFQMPTLASGWDVNKEKNNGAILFVKNGLYYLGIM |
| | PKQKGRYKALSFEPTEKTSEGFDKMYYDYFPDAAKMIPKCSTQLKAVTAHFQTHTTPILLS |
| | NNFIEPLEITKEIYDLNNPEKEPKKFQTAYAKKTGDQKGYREALCKWIDFTRDFLSKYTKTT |
| | SIDLSSLRPSSQYKDLGEYYAELNPLLYHISFQRIAEKEIMDAVETGKLYLFQIYNKDFAKGH |
| | HGKPNLHTLYWTGLFSPENLAKTSIKLNGQAELFYRPKSRMKRMAHRLGEKMLNKKLKDQ |
| | KTPIPDTLYQELYDYVNHRLSHDLSDEARALLPNVITKEVSHEIIKDRRFTSDKFFFHVPITLN |
| | YQAANSPSKFNQRVNAYLKEHPETPIIGIDRGERNLIYITVIDSTGKILEQRSLNTIQQFDYQK |
| | KLDNREKERVAARQAWSVVGTIKDLKQGYLSQVIHEIVDLMIHYQAVVVLENLNFGFKSK |
| | RTGIAEKAVYQQFEKMLIDKLNCLVLKDYPAEKVGGVLNPYQLTDQFTSFAKMGTQSGFLF |
| | YVPAPYTSKIDPLTGFVDPFVWKTIKNHESRKHFLEGFDFLHYDVKTGDFILHFKMNRNLSF |
| | QRGLPGFMPAWDIVFEKNETQFDAKGTPFIAGKRIVPVIENHRFTGRYRDLYPANELIALLEE |
| | KGIVFRDGSNILPKLLENDDSHAIDTMVALIRSVLQMANSNAATGEDYINSPVRDLNGVCFD |
| | SRFQNPEWPMDADANGAYHIALKGQLLLNHLKESKDLKLQNGISNQDWLAYIQELRN |
| | (SEQ ID NO: 46) |
| LbCas12a | MNYKTGLEDFIGKESLSKTLRNALIPTESTKIHMEEMGVIRDDELRAEKQQELKEIMDDYYR |
| (previously | TFIEEKLGQIQGIQWNSLFQKMEETMEDISVRKDLDKIQNEKRKEICCYFTSDKRFKDLFNA |
| known as | KLITDILPNFIKDNKEYTEEEKAEKEQTRVLFQRFATAFTNYFNQRRNNFSEDNISTAISFRIV |
| Cpf1) | NENSEIHLQNMRAFQRIEQQYPEEVCGMEEEYKDMLQEWQMKHIYSVDFYDRELTQPGIEY |
| Lachnospiraceae | YNGICGKINEHMNQFCQKNRINKNDFRMKKLHKQILCKKSSYYEIPFRFESDQEVYDALNEF |
| bacterium | IKTMKKKEIIRRCVHLGQECDDYDLGKIYISSNKYEQISNALYGSWDTIRKCIKEEYMDALP |
| GAM79 | GKGEKKEEKAEAAAKKEEYRSIADIDKIISLYGSEMDRTISAKKCITEICDMAGQISIDPLVCN |
| Ref Seq. | SDIKLLQNKEKTTEIKTILDSFLHVYQWGQTFIVSDIIEKDSYFYSELEDVLEDFEGITTLYNH |
| WP_119623382.1 | VRSYVTQKPYSTVKFKLHFGSPTLANGWSQSKEYDNNAILLMRDQKFYLGIFNVRNKPDK |
| | QIIKGHEKEEKGDYKKMIYNLLPGPSKMLPKVFITSRSGQETYKPSKHILDGYNEKRHIKSSP |
| | KFDLGYCWDLIDYYKECIHKHPDWKNYDFHFSDTKDYEDISGFYREVEMQGYQIKWTYIS |
| | ADEIQKLDEKGQIFLFQIYNKDFSVHSTGKDNLHTMYLKNLFSEENLKDIVLKLNGEAELFF |
| | RKASIKTPIVHKKGSVLVNRSYTQTVGNKEIRVSIPEEYYTEIYNYLNHIGKGKLSSEAQRYL |
| | DEGKIKSFTATKDIVKNYRYCCDHYFLHLPITINFKAKSDVAVNERTLAYIAKKEDIHIGIDR |
| | GERNLLYISVVDVHGNIREQRSFNIVNGYDYQQKLKDREKSRDAARKNWEEIEKIKELKEG |
| | YLSMVIHYIAQLVVKYNAVVAMEDLNYGFKTGRFKVERQVYQKFETMLIEKLHYLVFKDR |
| | EVCEEGGVLRGYQLTYIPESLKKVGKQCGFIFYVPAGYTSKIDPTTGFVNLESFKNLTNRESR |
| | QDFVGKFDEIRYDRDKKMFEFSFDYNNYIKKGTILASTKWKVYTNGTRLKRIVVNGKYTSQ |
| | SMEVELTDAMEKMLQRAGIEYHDGKDLKGQIVEKGIEAEIIDIFRLTVQMRNSRSESEDREY |
| | DRLISPVLNDKGEFFDTATADKTLPQDADANGAYCIALKGLYEVKQIKENWKENEQFPRNK. |
| | LVQDNKTWFDFMQKKRYL (SEQ ID NO: 47) |
| PcCas12a- | MAKNFEDFKRLYSLSKTLRFEAKPIGATLDNIVKSGLLDEDEHRAASYVKVKKLIDEYHKV |
| previously | FIDRVLDDGCLPLENKGNNNSLAEYYESYVSRAQDEDAKKKFKEIQQNLRSVIAKKLTEDK |
| known at | AYANLFGNKLIESYKDKEDKKKIIDSDLIQFINTAESTQLDSMSQDEAKELVKEFWGFVTYF |
| Cpf1 | YGFFDNRKNMYTAEEKSTGIAYRLVNENLPKFIDNIEAFNRAITRPEIQENMGVLYSDESEYL |
| Prevotella copri | NVESIQEMFQLDYYNMLLTQKQIDVYNAIIGGKTDDEHDVKIKGINEYINLYNQQHKDDKL |
| Ref Seq. | PKLKALFKQILSDRNAISWLPEEFNSDQEVLNAIKDCYERLAENVLGDKVLKSLLGSLADYS |
| WP_119227726.1 | LDGIFIRNDLQLTDISQKMFGNWGVIQNAIMQNIKRVAPARKHKESEEDYEKRIAGIFKKAD |
| | SFSISYINDCLNEADPNNAYFVENYFATFGAVNTPTMQRENLFALVQNAYTEVAALLHSDY |
| | PTVKHLAQDKANVSKIKALLDAIKSLQHFVKPLLGKGDESDKDERFYGELASLWAELDTVT |
| | PLYNMIRNYMTRKPYSQKKIKLNFENPQLLGGWDANKEKDYATIILRRNGLYYLAIMDKDS |
| | RKLLGKAMPSDGECYEKMVYKFFKDVTTMIPKCSTQLKDVQAYFKVNTDDYVLNSKAFN |
| | KPLTITKEVEDLNNVLYGKYKKFQKGYLTATGDNVGYTHAVNVWIKFCMDELNSYDSTCI |
| | YDESSLKPESYLSLDAFYQDANLLLYKLSFARASVSYINQLVEEGKMYLFQIYNKDFSEYSK |
| | GTPNMHTLYWKALFDERNLADVVYKLNGQAEMFYRKKSIENTHPTHPANHPILNKNKDNK |
| | KKESLFDYDLIKDRRYTVDKFMFHVPITMNFKSVGSENINQDVKAYLRHADDMHIIGIDRG |
| | ERHLLYLVVIDLQGNIKEQYSLNEIVNEYNGNTYHTNYHDLLDVREEERLKARQSWQTIENI |
| | KELKEGYLSQVIHKITQLMVRYHAIVVLEDLSKGFMRSRQKVEKQVYQKFEKMLIDKLNYL |
| | VDKKTDVSTPGGLLNAYQLTCKSDSSQKLGKQSGFLFYIPAWNTSKIDPVTGFVNLLDTHSL |
| | NSKEKIKAFFSKFDAIRYNKDKKWFEFNLDYDKFGKKAEDTRTKWTLCTRGMRIDTFRNKE |
| | KNSQWDNQEVDLTTEMKSLLEHYYIDIHGNLKDAISAQTDKAFFTGLLHILKLTLQMRNSIT |
| | GTETDYLVSPVADENGIFYDSRSCGNQLPENADANGAYNIARKGLMLIEQIKNAEDLNNVK |
| | FDISNKAWINFAQQKPYKNG (SEQ ID NO: 48) |
| ErCas12a- | MFSAKLISDILPEFVIHNNNYSASEKEEKTQVIKLESRFATSFKDYFKNRANCESANDISSSSC |
| previously | HRIVNDNAEIFFSNALVYRRIVKNLSNDDINKISGDMKDSLKEMSLEEIYSYEKYGEFITQEGI |
| known at | SFYNDICGKVNLFMNLYCQKNKENKNLYKLRKLHKQILCIADTSYEVPYKFESDEEVYQSV |
| Cpf1 | NGFLDNISSKHIVERLRKIGENYNGYNLDKIYIVSKFYESVSQKTYRDWETINTALEIHYNNI |
| Eubacterium | LPGNGKSKADKVKKAVKNDLQKSITEINELVSNYKLCPDDNIKAETYIHEISHILNNFEAQEL |
| rectale | KYNPEIHLVESELKASELKNVLDVIMNAFHWCSVFMTEELVDKDNNFYAELEEIYDEIYPVI |
| Ref Seq. | SLYNLVRNYVTQKPYSTKKIKLNFGIPTLADGWSKSKEYSNNAIILMRDNLYYLGIFNAKNK |
| WP_119223642.1 | PDKKIIEGNTSENKGDYKKMIYNLLPGPNKMIPKVFLSSKTGVETYKPSAYILEGYKQNKHL |
| | KSSKDFDITFCHDLIDYFKNCIAIHPEWKNFGFDESDTSTYEDISGFYREVELQGYKIDWTYIS |
| | EKDIDLLQEKGQLYLFQIYNKDFSKKSSGNDNLHTMYLKNLFSEENLKDIVLKLNGEAEIFF |
| | RKSSIKNPIIHKKGSILVNRTYEAEEKDQFGNIQIVRKTIPENIYQELYKYFNDKSDKELSDEA |
| | AKLKNVVGHHEAATNIVKDYRYTYDKYFLHMPITINFKANKTSFINDRILQYIAKEKDLHVI |
| | GIDRGERNLIYVSVIDTCGNIVEQKSFNIVNGYDYQIKLKQQEGARQIARKEWKEIGKIKEIK |
| | EGYLSLVIHEISKMVIKYNAIIAMEDLSYGFKKGRFKVERQVYQKFETMLINKLNYLVFKDIS |
| | ITENGGLLKGYQLTYIPDKLKNVGHQCGCIFYVPAAYTSKIDPTTGFVNIFKFKDLTVDAKR |
| | EFIKKFDSIRYDSDKNLFCFTFDYNNFITQNTVMSKSSWSVYTYGVRIKRRFVNGRESNESDT |
| | IDITKDMEKTLEMTDINWRDGHDLRQDIIDYEIVQHIFEIFKLTVQMRNSLSELEDRDYDRLI |
| | SPVLNENNIFYDSAKAGDALPKDADANGAYCIALKGLYEIKQITENWKEDGKFSRDKLKISN |
| | KDWFDFIQNKRYL (SEQ ID NO: 49) |
| CsCas12a | MNYKTGLEDFIGKESLSKTLRNALIPTESTKIHMEEMGVIRDDELRAEKQQELKEIMDDYYR |
| previously | AFIEEKLGQIQGIQWNSLFQKMEETMEDISVRKDLDKIQNEKRKEICCYFTSDKRFKDLFNA |
| known at | KLITDILPNFIKDNKEYTEEEKAEKEQTRVLFQRFATAFTNYFNQRRNNFSEDNISTAISFRIV |
| Cpf1 | NENSEIHLQNMRAFQRIEQQYPEEVCGMEEEYKDMLQEWQMKHIYLVDFYDRVLTQPGIE |
| Clostridium | YYNGICGKINEHMNQFCQKNRINKNDFRMKKLHKQILCKKSSYYEIPFRFESDQEVYDALN |
| sp. AF34- | EFIKTMKEKEIICRCVHLGQKCDDYDLGKIYISSNKYEQISNALYGSWDTIRKCIKEEYMDAL |
| 10BHRef | PGKGEKKEEKAEAAAKKEEYRSIADIDKIISLYGSEMDRTISAKKCITEICDMAGQISTDPLV |
| Seq. | CNSDIKLLQNKEKTTEIKTILDSFLHVYQWGQTFIVSDIIEKDSYFYSELEDVLEDFEGITTLY |
| WP_118538418.1 | NHVRSYVTQKPYSTVKFKLHFGSPTLANGWSQSKEYDNNAILLMRDQKFYLGIFNVRNKP |
| | DKQIIKGHEKEEKGDYKKMIYNLLPGPSKMLPKVFITSRSGQETYKPSKHILDGYNEKRHIK |
| | SSPKFDLGYCWDLIDYYKECIHKHPDWKNYDFHFSDTKDYEDISGFYREVEMQGYQIKWT |
| | YISADEIQKLDEKGQIFLFQIYNKDFSVHSTGKDNLHTMYLKNLFSEENLKDIVLKLNGEAEL |
| | FFRKASIKTPVVHKKGSVLVNRSYTQTVGDKEIRVSIPEEYYTEIYNYLNHIGRGKLSTEAQR |
| | YLEERKIKSFTATKDIVKNYRYCCDHYFLHLPITINFKAKSDIAVNERTLAYIAKKEDIHIIGID |
| | RGERNLLYISVVDVHGNIREQRSFNIVNGYDYQQKLKDREKSRDAARKNWEEIEKIKELKE |
| | GYLSMVIHYIAQLVVKYNAVVAMEDLNYGFKTGRFKVERQVYQKFETMLIEKLHYLVFKD |
| | REVCEEGGVLRGYQLTYIPESLKKVGKQCGFIFYVPAGYTSKIDPTTGFVNLFSFKNLTNRES |
| | RQDFVGKFDEIRYDRDKKMFEFSFDYNNYIKKGTMLASTKWKVYTNGTRLKRIVVNGKYT |
| | SQSMEVELTDAMEKMLQRAGIEYHDGKDLKGQIVEKGIEAEIIDIFRLTVQMRNSRSESEDR |
| | EYDRLISPVLNDKGEFFDTATADKTLPQDADANGAYCIALKQLYEVKQIKENWKENEQFPR |
| | NKLVQDNKTWFDFMQKKRYL (SEQ ID NO: 50) |
| BhCas12b | MATRSFILKIEPNEEVKKGLWKTHEVINHGIAYYMNILKLIRQEAIYEHHEQDPKNPKKVSK |
| Bacillus hisashii | AEIQAELWDFVLKMQKCNSFTHEVDKDEVENILRELYEELVPSSVEKKGEANQLSNKFLYP |
| Ref Seq. | LVDPNSQSGKGTASSGRKPRWYNIKIAGDPSWEEEKKKWEEDKKKDPLAKILGKLAEYGLI |
| WP_095142515.1 | PLFIPYTDSNEPIVKEIKWMEKSRNQSVRRLDKDMFIQALERFLSWESWNLKVKEEYEKVEK |
| | EYKTLEERIKEDIQALKALEQYEKERQEQLLRDTLNTNEYRLSKRGLRGWREIIQKWLKMD |
| | JENEPSEKYLEVFKDYQRKHPREAGDYSVYEFLSKKENHFIWRNHPEYPYLYATFCEIDKKK |
| | KDAKQQATFTLADPINHPLWVRFEERSGSNLNKYRILTEQLHTEKLKKKLTVQLDRLIYPTE |
| | SGGWEEKGKVDIVLLPSRQFYNQIFLDIEEKGKHAFTYKDESIKFPLKGTLGGARVQFDRDH |
| | LRRYPHKVESGNVGRIYFNMTVNIEPTESPVSKSLKIHRDDFPKVVNFKPKELTEWIKDSKG |
| | KKLKSGIESLEIGLRVMSIDLGQRQAAAASIFEVVDQKPDIEGKLFFPIKGTELYAVHRASFNI |
| | KLPGETLVKSREVLRKAREDNLKLMNQKLNFLRNVLHFQQFEDITEREKRVTKWISRQENS |
| | DVPLVYQDELIQIRELMYKPYKDWVAFLKQLHKRLEVEIGKEVKHWRKSLSDGRKGLYGIS |
| | LKNIDEIDRTRKFLLRWSLRPTEPGEVRRLEPGQRFAIDQLNHLNALKEDRLKKMANTIIMH |
| | ALGYCYDVRKKKWQAKNPACQIILFEDLSNYNPYEERSRFENSKLMKWSRREIPRQVALQG |
| | EIYGLQVGEVGAQFSSRFHAKTGSPGIRCSVVTKEKLQDNRFFKNLQREGRLTLDKIAVLKE |
| | GDLYPDKGGEKFISLSKDRKCVTTHADINAAQNLQKRFWTRTHGFYKVYCKAYQVDGQTV |
| | YIPESKDQKQKIIEEFGEGYFILKDGVYEWVNAGKLKIKKGSSKQSSSELVDSDILKDSFDLA |
| | SELKGEKLMLYRDPSGNVFPSDKWMAAGVFFGKLERILISKLTNQYSISTIEDDSSKQSM |
| | (SEQ ID NO: 44) |
| ThCas12b | MSEKTTQRAYTLRLNRASGECAVCQNNSCDCWHDALWATHKAVNRGAKAFGDWLLTLR |
| Thermomonas | GGLCHTLVEMEVPAKGNNPPQRPTDQERRDRRVLLALSWLSVEDEHGAPKEFIVATGRDS |
| hydrothermalis | ADDRAKKVEEKLREILEKRDFQEHEIDAWLQDCGPSLKAHIREDAVWVNRRALFDAAVERI |
| Ref Seq. | KTLTWEEAWDFLEPFFGTQYFAGIGDGKDKDDAEGPARQGEKAKDLVQKAGQWLSARFGI |
| WP_072754838 | GTGADEMSMAEAYEKIAKWASQAQNGDNGKATIEKLACALRPSEPPTLDTVLKCISGPGHK |
| | SATREYLKTLDKKSTVTQEDLNQLRKLADEDARNCRKKVGKKGKKPWADEVLKDVENSC |
| | ELTYLQDNSPARHREFSVMLDHAARRVSMAHSWIKKAEQRRRQFESDAQKLKNLQERAPS |
| | AVEWLDRFCESRSMTTGANTGSGYRIRKRAIEGWSYVVQAWAEASCDTEDKRIAAARKVQ |
| | ADPEIEKFGDIQLFEALAADEAICVWRDQEGTQNPSILIDYVTGKTAEHNQKRFKVPAYRHP |
| | DELRHPVFCDFGNSRWSIQFAIHKEIRDRDKGAKQDTRQLQNRHGLKMRLWNGRSMTDVN |
| | LHWSSKRLTADLALDQNPNPNPTEVTRADRLGRAASSAFDHVKIKNVFNEKEWNGRLQAP |
| | RAELDRIAKLEEQGKTEQAEKLRKRLRWYVSFSPCLSPSGPFIVYAGQHNIQPKRSGQYAPH |
| | AQANKGRARLAQLILSRLPDLRILSVDLGHRFAAACAVWETLSSDAFRREIQGLNVLAGGS |
| | GEGDLFLHVEMTGDDGKRRTVVYRRIGPDQLLDNTPHPAPWARLDRQFLIKLQGEDEGVR |
| | EASNEELWTVHKLEVEVGRTVPLIDRMVRSGFGKTEKQKERLKKLRELGWISAMPNEPSAE |
| | TDEKEGEIRSISRSVDELMSSALGTLRLALKRHGNRARIAFAMTADYKPMPGGQKYYFHEA |
| | KEASKNDDETKRRDNQIEFLQDALSLWHDLFSSPDWEDNEAKKLWQNHIATLPNYQTPEEI |
| | SAELKRVERNKKRKENRDKLRTAAKALAENDQLRQHLHDTWKERWESDDQQWKERLRSL |
| | KDWIFPRGKAEDNPSIRHVGGLSITRINTISGLYQILKAFKMRPEPDDLRKNIPQKGDDELEN |
| | FNRRLLEARDRLREQRVKQLASRIIEAALGVGRIKIPKNGKLPKRPRTTVDTPCHAVVIESLK |
| | TYRPDDLRTRRENRQLMQWSSAKVRKYLKEGCELYGLHFLEVPANYTSRQCSRTGLPGIRC |
| | DDVPTGDFLKAPWWRRAINTAREKNGGDAKDRFLVDLYDHLNNLQSKGEALPATVRVPR |
| | QGGNLFIAGAQLDDINKERRAIQADLNAAANIGLRALLDPDWRGRWWYVPCKDGTSEPAL |
| | DRIEGSTAFNDVRSLPTGDNSSRRAPREIENLWRDPSGDSLESGTWSPTRAYWDTVQSRVIE |
| | LLRRHAGLPTS (SEQ ID NO: 51) |
| LsCas12b | MSIRSFKLKLKTKSGVNAEQLRRGLWRTHQLINDGIAYYMNWLVLLRQEDLFIRNKETNEI |
| Laceyella sacchari | EKRSKEEIQAVLLERVHKQQQRNQWSGEVDEQTLLQALRQLYEEIVPSVIGKSGNASLKAR |
| WP_132221894.1 | FFLGPLVDPNNKTTKDVSKSGPTPKWKKMKDAGDPNWVQEYEKYMAERQTLVRLEEMGL |
| | IPLFPMYTDEVGDIHWLPQASGYTRTWDRDMFQQAIERLLSWESWNRRVRERRAQFEKKT |
| | HDFASRFSESDVQWMNKLREYEAQQEKSLEENAFAPNEPYALTKKALRGWERVYHSWMR |
| | LDSAASEEAYWQEVATCQTAMRGEFGDPAIYQFLAQKENHDIWRGYPERVIDFAELNHLQ |
| | RELRRAKEDATFTLPDSVDHPLWVRYEAPGGTNIHGYDLVQDTKRNLTLILDKFILPDENGS |
| | WHEVKKVPFSLAKSKQFHRQVWLQEEQKQKKREVVFYDYSTNLPHLGTLAGAKLQWDRN |
| | FLNKRTQQQIEETGEIGKVFFNISVDVRPAVEVKNGRLQNGLGKALTVLTHPDGTKIVTGW |
| | KAEQLEKWVGESGRVSSLGLDSLSEGLRVMSIDLGQRTSATVSVFEITKEAPDNPYKFFYQL |
| | EGTEMFAVHQRSFLLALPGENPPQKIKQMREIRWKERNRIKQQVDQLSAILRLHKKVNEDE |
| | RIQAIDKLLQKVASWQLNEEIATAWNQALSQLYSKAKENDLQWNQAIKNAHHQLEPVVGK |
| | QISLWRKDLSTGRQGIAGLSLWSIEELEATKKLLTRWSKRSREPGVVKRIERFETFAKQIQHH |
| | INQVKENRLKQLANLIVMTALGYKYDQEQKKWIEVYPACQVVLFENLRSYRFSFERSRREN |
| | KKLMEWSHRSIPKLVQMQGELFGLQVADVYAAYSSRYHGRTGAPGIRCHALTEADLRNET |
| | NIIHELIEAGFIKEEHRPYLQQGDLVPWSGGELFATLQKPYDNPRILTLHADINAAQNIQKRF |
| | WHPSMWFRVNCESVMEGEIVTYVPKNKTVHKKQGKTFRFVKVEGSDVYEWAKWSKNRN |
| | KNTFSSITERKPPSSMILFRDPSGTFFKEQEWVEQKTFWGKVQSMIQAYMKKTIVQRMEE |
| | (SEQ ID NO: 52) |
| DtCas12b | MVLGRKDDTAELRRALWTTHEHVNLAVAEVERVLLRCRGRSYWTLDRRGDPVHVPESQV |
| Dsulfonatronum | AEDALAMAREAQRRNGWPVVGEDEEILLALRYLYEQIVPSCLLDDLGKPLKGDAQKIGTN |
| thiodismutans | YAGPLFDSDTCRRDEGKDVACCGPFHEVAGKYLGALPEWATPISKQEFDGKDASHLRFKA |
| WP_031386437 | TGGDDAFFRVSIEKANAWYEDPANQDALKNKAYNKDDWKKEKDKGISSWAVKYIQKQLQ |
| | LGQDPRTEVRRKLWLELGLLPLFIPVFDKTMVGNLWNRLAVRLALAHLLSWESWNHRAVQ |
| | DQALARAKRDELAALFLGMEDGFAGLREYELRRNESIKQHAFEPVDRPYVVSGRALRSWT |
| | RVREEWLRHGDTQESRKNICNRLQDRLRGKFGDPDVFHWLAEDGQEALWKERDCVTSFSL |
| | LNDADGLLEKRKGYALMTFADARLHPRWAMYEAPGGSNLRTYQIRKTENGLWADVVLLS |
| | PRNESAAVEEKTFNVRLAPSGQLSNVSFDQIQKGSKMVGRCRYQSANQQFEGLLGGAEILF |
| | DRKRIANEQHGATDLASKPGHVWFKLTLDVRPQAPQGWLDGKGRPALPPEAKHFKTALSN |
| | KSKFADQVRPGLRVLSVDLGVRSFAACSVFELVRGGPDQGTYFPAADGRTVDDPEKLWAK |
| | HERSFKITLPGENPSRKEEIARRAAMEELRSLNGDIRRLKAILRLSVLQEDDPRTEHLRLFME |
| | AIVDDPAKSALNAELFKGFGDDRFRSTPDLWKQHCHFFHDKAEKVVAERFSRWRTETRPKS |
| | SSWQDWRERRGYAGGKSYWAVTYLEAVRGLILRWNMRGRTYGEVNRQDKKQFGTVASA |
| | LLHHINQLKEDRIKTGADMIIQAARGFVPRKNGAGWVQVHEPCRLILFEDLARYRFRTDRSR |
| | RENSRLMRWSHREIVNEVGMQGELYGLHVDTTEAGFSSRYLASSGAPGVRCRHLVEEDFH |
| | DGLPGMHLVGELDWLLPKDKDRTANEARRLLGGMVRPGMLVPWDGGELFATLNAASQL |
| | HVIHADINAAQNLQRRFWGRCGEAIRIVCNQLSVDGSTRYEMAKAPKARLLGALQQLKNG |
| | DAPFHLTSIPNSQKPENSYVMTPTNAGKKYRAGPGEKSSGEEDELALDIVEQAEELAQGRKT |
| | FFRDPSGVFFAPDRWLPSEIYWSRIRRRIWQVTLERNSSGRQERAEMDEMPY (SEQ ID NO: |
| | 53) |

The prime editors utilized in the methods and compositions described herein may also comprise Cas12a (Cpf1) (dCpf1) variants that may be used as a guide nucleotide sequence-programmable DNA-binding protein domain. The Cas12a (Cpf1) protein has a RuvC-like endonuclease domain that is similar to the RuvC domain of Cas9 but does not have a HNH endonuclease domain, and the N-terminal of Cas12a (Cpf1) does not have the alfa-helical recognition lobe of Cas9. It was shown in Zetsche et al., Cell, 163, 759-771, 2015 (which is incorporated herein by reference) that, the RuvC-like domain of Cas12a (Cpf1) is responsible for cleaving both DNA strands and inactivation of the RuvC-like domain inactivates Cas12a (Cpf1) nuclease activity.

In some embodiments, the napDNAbp is a single effector of a microbial CRISPR-Cas system. Single effectors of microbial CRISPR-Cas systems include, without limitation, Cas9, Cas12a (Cpf1), Cas12b1 (C2c1), Cas13a (C2c2), and Cas12c (C2c3). Typically, microbial CRISPR-Cas systems are divided into Class 1 and Class 2 systems. Class 1 systems have multi-subunit effector complexes, while Class 2 systems have a single protein effector. For example, Cas9 and Cas12a (Cpf1) are Class 2 effectors. In addition to Cas9 and Cas12a (Cpf1), three distinct Class 2 CRISPR-Cas systems (Cas12b1, Cas13a, and Cas12c) have been described by Shmakov et al., “Discovery and Functional Characterization of Diverse Class 2 CRISPR Cas Systems”, Mol. Cell, 2015 Nov. 5; 60(3): 385-397, the entire contents of which are hereby incorporated by reference.

Effectors of two of the systems, Cas12b1 and Cas12c, contain RuvC-like endonuclease domains related to Cas12a. A third system, Cas13a contains an effector with two predicted HEPN RNase domains. Production of mature CRISPR RNA is tracrRNA-independent, unlike production of CRISPR RNA by Cas12b1, Cas12b1 depends on both CRISPR RNA and tracrRNA for DNA cleavage. Bacterial Cas13a has been shown to possess a unique RNase activity for CRISPR RNA maturation distinct from its RNA-activated single-stranded RNA degradation activity. These RNase functions are different from each other and from the CRISPR RNA-processing behavior of Cas12a. See, e.g., East-Seletsky, et al., “Two distinct RNase activities of CRISPR-Cas13a enable guide-RNA processing and RNA detection”, Nature, 2016 Oct. 13; 538(7624):270-273, the entire contents of which are hereby incorporated by reference. In vitro biochemical analysis of Cas13a in Leptotrichia shahii has shown that Cas13a is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), the entire contents of which are hereby incorporated by reference.

The crystal structure of Alicyclobaccillus acidoterrastris Cas12b1 (AacC2c1) has been reported in complex with a chimeric single-molecule guide RNA (sgRNA). See e.g., Liu el al., “C2c1-sgRNA Complex Structure Reveals RNA-Guided DNA Cleavage Mechanism”, Mol. Cell, 2017 Jan. 19:65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell. 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems. In some embodiments, the napDNAbp may be a C2c1, a C2c2, or a C2c3 protein. In some embodiments, the napDNAbp is a C2c1 protein. In some embodiments, the napDNAbp is a Cas13a protein. In some embodiments, the napDNAbp is a Cas12c protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12b1 (C2c1), Cas13a (C2c2), or Cas12c (C2c3) protein. In some embodiments, the napDNAbp is a naturally-occurring Cas12b1 (C2c1), Cas13a (C2c2), or Cas12c (C2c3) protein.

### H, Cas9 Circular Permutants

In various embodiments, the prime editors utilized in the methods and compositions disclosed herein may comprise a circular permutant of Cas9.

The term “circularly permuted Cas9” or “circular permutant” of Cas9 or “CP-Cas9”) refers to any Cas9 protein, or variant thereof, that occurs or has been modified or engineered as a circular permutant variant, which means the N-terminus and the C-terminus of a Cas9 protein (e.g., a wild type Cas9 protein) have been topically rearranged. Such circularly permuted Cas9 proteins, or variants thereof, retain the ability to bind DNA when complexed with a guide RNA (gRNA). See, Oakes et al., “Protein Engineering of Cas9 for enhanced function,” Methods Enzymol. 2014, 546: 491-511 and Oakes et al., “CRISPR-Cas9 Circular Permutants as Programmable Scaffolds for Genome Modification,” Cell, Jan. 10, 2019, 176: 254-267, each of are incorporated herein by reference. The instant disclosure contemplates any previously known CP-Cas9 or use a new CP-Cas9 so long as the resulting circularly permuted protein retains the ability to bind DNA when complexed with a guide RNA (gRNA).

Any of the Cas9 proteins described herein, including any variant, ortholog, or any engineered or naturally occurring Cas9 or equivalent thereof, may be reconfigured as a circular permutant variant.

In various embodiments, the circular permutants of Cas9 may have the following structure: N-terminus-[original C-terminus]-[optional linker]-[original N-terminus]-C-terminus.

As an example, the present disclosure contemplates the following circular permutants of canonical S. pyogenes Cas9 (1368 amino acids of UniProtKB—Q99ZW2 (CAS9_STRP1) (numbering is based on the amino acid position in SEQ ID NO: 2)):
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus; or
N-terminus-[10-1368]-[optional linker]-[1-9]-C-terminus, or the corresponding circular permutants of other Cas9 proteins (including other Cas9 orthologs, variants, etc.).

In particular embodiments, the circular permutant Cas9 has the following structure (based on S. pyogenes Cas9 (1368 amino acids of UniProtKB—Q99ZW2 (CAS9_STRP1) (numbering is based on the amino acid position in SEQ ID NO: 2):
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc.).

In still other embodiments, the circular permeant Cas9 has the following structure (based on S. pyogenes Cas9 (1368 amino acids of UniProtKB—Q99ZW2 (CAS9_STRP1) (numbering is based on the amino acid position in SEQ ID NO: 2):
N-terminus-[103-1368]-[optional linker]-[1-102]-C-terminus;
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc.).

In some embodiments, the circular permutant can be formed by linking a C-terminal fragment of a Cas9 to an N-terminal fragment of a Cas9, either directly or by using a linker, such as an amino acid linker. In some embodiments, The C-terminal fragment may correspond to the C-terminal 95% or more of the amino acids of a Cas9 (e.g., amino acids about 1300-1368), or the C-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., any one of SEQ ID NOs: 54-63). The N-terminal portion may correspond to the N-terminal 95% or more of the amino acids of a Cas9 (e.g., amino acids about 1-1300), or the N-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., of SEQ ID NO: 2).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 of SEQ ID NO: 2). In some embodiments, the C-terminal fragment that is rearranged to the N-terminus includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or I % of the amino acids of a Cas9 (e.g., the Cas9 of SEQ ID NO: 2). In some embodiments, the C-terminal fragment that is rearranged to the N-terminus includes or corresponds to the C-terminal 410 residues or less of a Cas9 (e.g., the Cas9 of SEQ ID NO: 2). In some embodiments, the C-terminal portion that is rearranged to the N-terminus includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., the Cas9 of SEQ ID NO: 2). In some embodiments, the C-terminal portion that is rearranged to the N-terminus includes or corresponds to the C-terminal 357, 341, 328, 120, or 69 residues of a Cas9 (e.g., the Cas9 of SEQ ID NO: 2).

In other embodiments, circular permutant Cas9 variants may be defined as a topological rearrangement of a Cas9 primary structure based on the following method, which is based on S. pyogenes Cas9 of SEQ ID NO: 2: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (relative the S. pyogenes Cas9 of SEQ ID NO: 2) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²¹⁰, Cas9-CP²⁷⁰, Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 2, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

Exemplary CP-Cas9 amino acid sequences, based on the Cas9 of SEQ ID NO: 2, are provided below in which linker sequences are indicated by underlining and optional methionine (M) residues are indicated in bold. It should be appreciated that the disclosure provides CP-Cas9 sequences that do not include a linker sequence or that include different linker sequences. It should be appreciated that CP-Cas9 sequences may be based on Cas9 sequences other than that of SEQ ID NO: 2 and any examples provided herein are not meant to be limiting. Exemplary CP-Cas9 sequences are as follows:

| | | SEQ |
|---|---|---|
| CP name | Sequence | ID NO: |
| CP1012 | DYKVYDVRKMIAKSEQEIGKATAKYFFYSNIMNFFKTEITLANGEIRKRPLIETNGETGE | 54 |
| | IVWDKGRDFATVRKVLSMPQVNIVKKTEVQTGGFSKESILPKRNSDKLIARKKDWDPK | |
| | KYGGFDSPTVAYSVLVVAKVEKGKSKKLKSVKELLGITIMERSSFEKNPIDFLEAKGYK | |
| | EVKKDLIIKLPKYSLFELENGRKRMLASAGELQKGNELALPSKYVNFLYLASHYEKLK | |
| | GSPEDNEQKQLFVEQHKHYLDEIIEQISEFSKRVILADANLDKVLSAYNKHRDKPIREQ | |
| | AENIIHLFTLINLGAPAAFKYFDTTIDRKRYTSTKEVLDATLIHQSITGLYETRIDLSQLG | |
| | GDGGSGGSGGSGGSGGSGGSGGDKKYSIGLAIGTNSVGWAVITDEYKVPSKKFKVLG | |
| | NTDRHSIKKNLIGALLFDSGETAEATRLKRTARRRYTRRKNRICYLQEIFSNEMAKVDD | |
| | SFFHRLEESFLVEEDKKHERHPIFGNIVDEVAYHEKYPTIYHLRKKLVDSTDKADLRLIY | |
| | LALAHMIKFRGHFLIEGDLNPDNSDVDKLFIQLVQTYNQLFEENPINASGVDAKAILSA | |
| | RLSKSRRLENLIAQLPGEKKNGLFGNLIALSLGLTPNFKSNFDLAEDAKLQLSKDTYDD | |
| | DLDNLLAQIGDQYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPILEKMDGTEELLV | |
| | KLNREDLLRKQRTFDNGSIPHQIHLGELHAILRRQEDFYPFLKDNREKIEKILTFRIPYYV | |
| | GPLARGNSRFAWMTRKSEETITPWNFEEVVDKGASAQSFIERMTNFDKNLPNEK VLPK | |
| | HSLLYEYFTVYNELTKVKYVTEGMRKPAFLSGEQKKAIVDLLFKTNRKVTVKOLKED | |
| | YFKKIECFDSVEISGVEDRFNASLGTYHDLLKIIKDKDFLDNEENEDILEDIVLTLTLFED | |
| | REMIEERLKTYAHLFDDKVMKOLKRRRYTGWGRLSRKLINGIRDKQSGKTILDFLKSD | |
| | GFANRNEMQLIHDDSLTFKEDIQKAQVSGQGDSLHEHIANLAGSPAIKKGILQTVKVVD | |
| | ELVKVMGRHKPENIVIEMARENQTTQKGQKNSRERMKRIEEGIKELGSQILKEHPVEN | |
| | TQLQNEKLYLYYLONGRDMYVDQELDINRLSDYDVDHIVPQSFLKDDSIDNKVLTRSD | |
| | KNROKSDNVPSEEVVKKMKNYWRQLLNAKLITQRKFDNLTKAERGGLSELDKAGFIK | |
| | RQLVETROITKHVAQILDSRMNTKYDENDKLIREVKVITLKSKLVSDFRKDFQFYKVRE | |
| | INNYHHAHDAYLNAVVGTALIKKYPKLESEFVYG | |
| CP1028 | EIGKATAKYFFYSNIMNFFKTEITLANGEIRKRPLIETNGETGEIVWDKGRDFATVRKV | 55 |
| | LSMPQVNIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLIIKLPKYSL | |
| | FELENGRKRMLASAGELOKGNELALPSKYVNFLYLASHYEKLKGSPEDNEQKQLFVE | |
| | QHKHYLDEIIEQISEFSKRVILADANLDKVLSAYNKHRDKPIREQAENIIHLFTLINLGA | |
| | PAAFKYFDTTIDRKRYTSTKEVLDATLIHQSITGLYETRIDLSQLGGDGGSGGSGGSGG | |
| | SGGSGGSGGMDKKYSIGLAIGTNSVGWAVITDEYKVPSKKFKVLGNTDRHSIKKNLIG | |
| | ALLFDSGETAEATRLKRTARRRYTRRKNRICYLQEIFSNEMAKVDDSFFHRLEESFLVE | |
| | EDKKHERHPIFGNIVDEVAYHEKYPTIYHLRKKLVDSTDKADLRLIYLALAHMIKFRG | |
| | HFLIEGDLNPDNSDVDKLFIQLVQTYNQLFEENPINASGVDAKAILSARLSKSRRLENLI | |
| | AQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQL | |
| | PEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFA | |
| | WMTRKSEETITPWNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKEDYFKKIECFDSV | |
| | EISGVEDRFNASLGTYHDLLKIIKDKDFLDNEENEDILEDIVLTLTLFEDREMIEERLKT | |
| | YAHLFDDKVMKOLKRRRYTGWGRLSRKLINGIRDKQSGKTILDFLKSDGFANRNFMQ | |
| | LIHDDSLTFKEDIQKAQVSGQGDSLHEHIANLAGSPAIKKGILQTVKVVDELVKVMGR | |
| | HKPENIVIEMARENOTTOKGQKNSRERMKRIEEGIKELGSQILKEHPVENTQLQNEKLY | |
| | LYYLONGRDMYVDQELDINRLSDYDVDHIVPQSFLKDDSIDNKVLTRSDKNRGKSDN | |
| | VPSEEVVKKMKNYWRQLLNAKLITORKEDNLTKAERGGLSELDKAGFIKRQLVETRQ | |
| | ITKHVAQILDSRMNTKYDENDKLIREVKVITLKSKLVSDFRKDFOFYKVREINNYHHA | |
| | HDAYLNAVVGTALIKKYPKLESEFVYGDYKVYDVRKMIAKSEQ | |
| CP1041 | NIMNFFKTEITLANGEIRKRPLIETNGETGEIVWDKGRDFATVRKVLSMPQVNIVKKTE | 56 |
| | VQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLAS | |
| | AGELQKGNELALPSKYVNFLYLASHYEKLKGSPEDNEQKQLFVEQHKHYLDEIIEQISE | |
| | FSKRVILADANLDKVLSAYNKHRDKPIREQAENIIHLFTLTNLGAPAAFKYFDTTIDRK | |
| | RYTSTKEVLDATLIHQSITGLYETRIDLSQLGGDGGSGGSGGSGGSGGSGGSGGDKKY | |
| | SIGLAIGTNSVGWAVITDEYKVPSKKFKVLGNTDRHSIKKNLIGALLFDSGETAEATRL | |
| | KRTARRRYTRRKNRICYLQEIFSNEMAKVDDSFFHRLEESFLVEEDKKHERHPIFGNIV | |
| | DEVAYHEKYPTIYHLRKKLVDSTDKADLRLIYLALAHMIKFRGHFLIEGDLNPDNSDV | |
| | DKLFIQLVQTYNQLFEENPINASGVDAKAILSARLSKSRRLENLIAQLPGEKKNGLFGN | |
| | LIALSLGLTPNFKSNFDLAEDAKLQLSKDTYDDDLDNLLAQIGDQYADLFLAAKNLSD | |
| | AILLSDILRVNTEITKAPLSASMIKRYDEHHQDLTLLKALVROQLPEKYKEIFFDQSKN | |
| | GYAGYIDGGASQEEFYKFIKPILEKMDGTEELLVKLNREDLLRKORTFDNGSIPHQIHL | |
| | GELHAILRRQEDFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISGVEDRENAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMARE | |
| | NOTTQKGQKNSRERMKRIEEGIKELGSQILKEHPVENTQLQNEKLYLYYLONGRDMY | |
| | VDQELDINRLSDYDVDHIVPQSFLKDDSIDNKVLTRSDKNRGKSDNVPSEEVVKKMK | |
| | NYWROLLNAKLITORKFDNLTKAERGGLSELDKAGFIKRQLVETRQITKHVAQILDSR | |
| | MNTKYDENDKLIREVKVITLKSKLVSDFRKDFQFYKVREINNYHHAHDAYLNAVVGT | |
| | ALIKKYPKLESEFVYGDYKVYDVRKMIAKSEQEIGKATAKYFFYS | |
| CP1249 | PEDNEQKQLFVEQHKHYLDEIIEQISEFSKRVILADANLDKVLSAYNKHRDKPIREQAE | 57 |
| | NIIHLFTLTNLGAPAAFKYFDTTIDRKRYTSTKEVLDATLIHQSITGLYETRIDLSQLGG | |
| | DGGSGGSGGSGGSGGSGGSGGMDKKYSIGLAIGTNSVGWAVITDEYKVPSKKFKVLG | |
| | NTDRHSIKKNLIGALLEDSGETAEATRLKRTARRRYTRRKNRICYLQEIFSNEMAKVD | |
| | DSFFHRLEESFLVEEDKKHERHPIFGNIVDEVAYHEKYPTIYHLRKKLVDSTDKADLRL | |
| | IYLALAHMIKFRGHFLIEGDLNPDNSDVDKLFIQLVQTYNQLFEENPINASGVDAKAIL | |
| | SARLSKSRRLENLIAQLPGEKKNGLFGNLIALSLGLTPNFKSNFDLAEDAKLQLSKDTY | |
| | DDDLDNLLAQIGDQYADLFLAAKNLSDAILLSDILRVNTEITKAPLSASMIKRYDEHHQ | |
| | DLTLLKALVRQQLPEKYKEIFFDQSKNGYAGYIDGGASQEEFYKFIKPILEKMDGTEEL | |
| | LVKLNREDLLRKQRTFDNGSIPHQIHLGELHAILRRQEDFYPFLKDNREKIEKILTFRIPY | |
| | YVGPLARGNSRFAWMTRKSEETITPWNFEEVVDKGASAQSFIERMTNFDKNLPNEKV | |
| | LPKHSLLYEYFTVYNELTKVKYVTEGMRKPAFLSGEQKKAIVDLLFKTNRKVTVKOL | |
| | KEDYFKKIECFDSVEISGVEDRFNASLGTYHDLLKIIKDKDELDNEENEDILEDIVLTLT | |
| | LFEDREMIEERLKTYAHLFDDKVMKOLKRRRYTGWGRLSRKLINGIRDKQSGKTILDF | |
| | LKSDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKEH | |
| | PVENTQLQNEKLYLYYLQNGRDMYVDQELDINRLSDYDVDHIVPQSFLKDDSIDNKV | |
| | LTRSDKNRGKSDNVPSEEVVKKMKNYWRQLLNAKLITORKFDNLTKAERGGLSELD | |
| | KAGFIKRQLVETRQITKHVAQILDSRMNTKYDENDKLIREVKVITLKSKLVSDFRKDFQ | |
| | FYKVREINNYHHAHDAYLNAVVGTALIKKYPKLESEFVYGDYKVYDVRKMIAKSEQE | |
| | IGKATAKYFFYSNIMNFFKTEITLANGEIRKRPLIETNGETGEIVWDKGRDFATVRKVL | |
| | SMPQVNIVKKTEVQTGGFSKESILPKRNSDKLIARKKDWDPKKYGGFDSPTVAYSVLV | |
| | VAKVEKGKSKKLKSVKELLGITIMERSSFEKNPIDFLEAKGYKEVKKDLIIKLPKYSLFE | |
| | LENGRKRMLASAGELOKGNELALPSKYVNFLYLASHYEKLKGS | |
| CP1300 | KPIREQAENIIHLFTLTNLGAPAAFKYFDTTIDRKRYTSTKEVLDATLIHQSITGLYETRI | 58 |
| | DLSQLGGDGGSGGSGGSGGSGGSGGSGGDKKYSIGLAIGTNSVGWAVITDEYKVPSK | |
| | KFKVLGNTDRHSIKKNLIGALLFDSGETAEATRLKRTARRRYTRRKNRICYLQEIFSNE | |
| | MAKVDDSFFHRLEESELVEEDKKHERHPIFGNIVDEVAYHEKYPTIYHLRKKLVDSTD | |
| | KADLRLIYLALAHMIKFRGHFLIEGDLNPDNSDVDKLFIQLVQTYNQLFEENPINASGV | |
| | DAKAILSARLSKSRRLENLIAQLPGEKKNGLFGNLIALSLGLTPNFKSNFDLAEDAKLQ | |
| | LSKDTYDDDLDNLLAQIGDQYADLFLAAKNLSDAILLSDILRVNTEITKAPLSASMIKR | |
| | YDEHHQDLTLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPFLKDNREKIE | |
| | KILTFRIPYYVGPLARGNSRFAWMTRKSEETITPWNFEEVVDKGASAQSFIERMTNEDK | |
| | NLPNEKVLPKHSLLYEYFTVYNELTKVKYVTEGMRKPAFLSGEQKKAIVDLLFKTNR | |
| | KVTVKOLKEDYFKKIECFDSVEISGVEDRENASLGTYHDLLKIIKDKDFLDNEENEDIL | |
| | EDIVLTLTLFEDREMIEERLKTYAHLEDDKVMKOLKRRRYTGWGRLSRKLINGIRDKQ | |
| | SGKTILDFLKSDGFANRNFMQLIHDDSLTFKEDIQKAQVSGQGDSLHEHIANLAGSPAI | |
| | KKGILQTVKVVDELVKVMGRHKPENIVIEMARENQTTQKGQKNSRERMKRIEEGIKE | |
| | LGSQILKEHPVENTQLQNEKLYLYYLQNGRDMYVDQELDINRLSDYDVDHIVPQSFLK | |
| | DDSIDNKVLTRSDKNRGKSDNVPSEEVVKKMKNYWROLLNAKLITQRKFDNLTKAE | |
| | RGGLSELDKAGFIKRQLVETROITKHVAQILDSRMNTKYDENDKLIREVKVITLKSKLV | |
| | SDFRKDFQFYKVREINNYHHAHDAYLNAVVGTALIKKYPKLESEFVYGDYKVYDVR | |
| | KMIAKSEQEIGKATAKYFFYSNIMNFFKTEITLANGEIRKRPLIETNGETGEIVWDKGR | |
| | DFATVRKVLSMPQVNIVKKTEVQTGGFSKESILPKRNSDKLIARKKDWDPKKYGGEDS | |
| | PTVAYSVLVVAKVEKGKSKKLKSVKELLGITIMERSSFEKNPIDFLEAKGYKEVKKDLI | |
| | IKLPKYSLFELENGRKRMLASAGELQKGNELALPSKYVNFLYLASHYEKLKGSPEDNE | |
| | QKQLFVEQHKHYLDEIIEQISEFSKRVILADANLDKVLSAYNKHRD | |

The Cas9 circular permutants may be useful in the prime editing constructs utilized in the methods and compositions described herein. Exemplary C-terminal fragments of Cas9, based on the Cas9 of SEQ ID NO: 2, which may be rearranged to an N-terminus of Cas9, are provided below. It should be appreciated that such C-terminal fragments of Cas9 are exemplary and are not meant to be limiting. These exemplary CP-Cas9 fragments have the following sequences:

| | | SEQ |
|---|---|---|
| CP name | Sequence | ID NO: |
| CP1012 | DYKVYDVRKMIAKSEQEIGKATAKYFFYSN | 59 |
| C- | IMNFFKTEITLANGEIRKRPLIETNGETGE | |
| terminal | IVWDKGRDFATVRKVLSMPQVNIVKKTEVQ | |
| fragment | TGGFSKESILPKRNSDKLIARKKDWDPKKY | |
| | GGFDSPTVAYSVLVVAKVEKGKSKKLKSVK | |
| | ELLGITIMERSSFEKNPIDFLEAKGYKEVK | |
| | KDLIIKLPKYSLFELENGRKRMLASAGELQ | |
| | KGNELALPSKYVNFLYLASHYEKLKGSPED | |
| | NEQKQLFVEQHKHYLDEIIEQISEFSKRVI | |
| | LADANLDKVLSAYNKHRDKPIREQAENIIH | |
| | LFTLTNLGAPAAFKYFDTTIDRKRYTSTKE | |
| | VLDATLIHQSITGLYETRIDLSQLGGD | |
| CP1028 | EIGKATAKYFFYSNIMNFFKTEITLANGEI | 60 |
| C- | RKRPLIETNGETGEIVWDKGRDFATVRKVL | |
| terminal | SMPQVNIVKKTEVQTGGFSKESILPKRNSD | |
| fragment | KLIARKKDWDPKKYGGEDSPTVAYSVLVVA | |
| | KVEKGKSKKLKSVKELLGITIMERSSFEKN | |
| | PIDFLEAKGYKEVKKDLIIKLPKYSLFELE | |
| | NGRKRMLASAGELQKGNELALPSKYVNFLY | |
| | LASHYEKLKGSPEDNEQKQLFVEQHKHYLD | |
| | EIIEQISEFSKRVILADANLDKVLSAYNKH | |
| | RDKPIREQAENIIHLFTLINLGAPAAFKYF | |
| | DTTIDRKRYTSTKEVLDATLIHQSITGLYE | |
| | TRIDLSQLGGD | |
| CP1041 | NIMNFFKTEITLANGEIRKRPLIETNGETG | 61 |
| C- | EIVWDKGRDFATVRKVLSMPQVNIVKKTEV | |
| terminal | QTGGFSKESILPKRNSDKLIARKKDWDPKK | |
| fragment | YGGFDSPTVAYSVLVVAKVEKGKSKKLKSV | |
| | KELLGITIMERSSFEKNPIDFLEAKGYKEV | |
| | KKDLIIKLPKYSLFELENGRKRMLASAGEL | |
| | QKGNELALPSKYVNFLYLASHYEKLKGSPE | |
| | DNEQKQLFVEQHKHYLDEIIEQISEFSKRV | |
| | ILADANLDKVLSAYNKHRDKPIREQAENII | |
| | HLFTLTNLGAPAAFKYEDTTIDRKRYTSTK | |
| | EVLDATLIHQSITGLYETRIDLSQLGGD | |
| CP1249 | PEDNEQKQLFVEQHKHYLDEIIEQISEFSK | 62 |
| C- | RVILADANLDKVLSAYNKHRDKPIREQAEN | |
| terminal | IHLFTLTNLGAPAAFKYFDTTIDRKRYTST | |
| fragment | KEVLDATLIHQSITGLYETRIDLSQLGGD | |
| CP1300 | KPIREQAENIIHLFTLTNLGAPAAFKYFDT | 63 |
| C- | TIDRKRYTSTKEVLDATLIHQSITGLYETR | |
| terminal | IDLSQLGGD | |
| fragment | | |

I, Cas9 Variants with Modified PAM Specificities

The prime editors utilized in the methods and compositions of the present disclosure may also comprise Cas9 variants with modified PAM specificities. Some aspects of this disclosure provide Cas9 proteins that exhibit activity on a target sequence that does not comprise the canonical PAM (5′-NGG-3′, where N is A, C. G, or T) at its 3′-end. In some embodiments, the Cas9 protein exhibits activity on a target sequence comprising a 5′-NGG-3′ PAM sequence at its 3′-end. In some embodiments, the Cas9 protein exhibits activity on a target sequence comprising a 5′-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′-NAT-3′ PAM sequence at its 3′-end. In still other embodiments, the Cas9 protein exhibits activity on a target sequence comprising a 5′-NAG-3′ PAM sequence at its 3′-end.

It should be appreciated that any of the amino acid mutations described herein, (e.g., A262T) from a first amino acid residue (e.g., A) to a second amino acid residue (e.g., T) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine (e.g., a A262T mutation) may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine; asparagine and glutamine; methionine and cysteine; aspartic acid and glutamic acid; and arginine and lysine. The skilled artisan would recognize that such conservative amino acid substitutions will likely have minor effects on protein structure and are likely to be well tolerated without compromising function. In some embodiments, any amino of the amino acid mutations provided herein from one amino acid to a threonine may be an amino acid mutation to a serine. In some embodiments, any amino of the amino acid mutations provided herein from one amino acid to an arginine may be an amino acid mutation to a lysine. In some embodiments, any amino of the amino acid mutations provided herein from one amino acid to an isoleucine, may be an amino acid mutation to an alanine, valine, methionine, or leucine. In some embodiments, any amino of the amino acid mutations provided herein from one amino acid to a lysine may be an amino acid mutation to an arginine. In some embodiments, any amino of the amino acid mutations provided herein from one amino acid to an aspartic acid may be an amino acid mutation to a glutamic acid or asparagine. In some embodiments, any amino of the amino acid mutations provided herein from one amino acid to a valine may be an amino acid mutation to an alanine, isoleucine, methionine, or leucine. In some embodiments, any amino of the amino acid mutations provided herein from one amino acid to a glycine may be an amino acid mutation to an alanine. It should be appreciated, however, that additional conserved amino acid residues would be recognized by the skilled artisan and any of the amino acid mutations to other conserved amino acid residues are also within the scope of this disclosure.

In some embodiments, the Cas9 protein comprises a combination of mutations that exhibit activity on a target sequence comprising a 5′-NAA-3′ PAM sequence at its 3′-end. In some embodiments, the combination of mutations are present in any one of the clones listed in Table 1. In some embodiments, the combination of mutations are conservative mutations of the clones listed in Table 1. In some embodiments, the Cas9 protein comprises the combination of mutations of any one of the Cas9 clones listed in Table 1.

TABLE 1

NAA PAM Clones
Mutations from wild-type SpCas9 (e.g., SEQ ID NO: 2)
D177N, K218R, D614N, D1135N, P1137S,
E1219V, A1320V, A1323D, R1333K
D177N, K218R, D614N, D1135N, E1219V,
Q1221H, H1264Y, A1320V, R1333K
A10T, I322V, S409I, E427G, G715C, D1135N,
E1219V, Q1221H, H1264Y, A1320V, R1333K
A367T, K710E, R1114G, D1135N, P1137S, E1219V,
Q1221H, H1264Y, A1320V, R1333K
A10T, I322V, S409I, E427G, R753G, D861N, D1135N,
K1188R, E1219V, Q1221H, H1264H, A1320V,
R1333K
A10T, I322V, S409I, E427G, R654L, V743I, R753G,
M1021T, D1135N, D1180G, K1211R, E1219V, Q1221H,
H1264Y, A1320V, R1333K
A10T, I322V, S409I, E427G, V743I, R753G, E762G,
D1135N, D1180G, K1211R, E1219V, Q1221H, H1264Y,
A1320V, R1333K
A10T, I322V, S409I, E427G, R753G, D1135N, D1180G,
K1211R, E1219V, Q1221H, H1264Y, S1274R,
A1320V, R1333K
A10T, I322V, S409I, E4270, A589S, R753G, D1135N,
E1219V, Q1221H, H1264H, A1320V, R1333K
A10T, I322V, S409I, E427G, R753G, E757K, G865G,
D1135N, E1219V, Q1221H, H1264Y, A1320V, R1333K
A10T, I322V, S409I, E427G, R654L, R753G, E757K,
D1135N, E1219V, Q1221H, H1264Y, A1320V, R1333K
A10T, I322V, S409I, E427G, K599R, M631A, R654L,
K673E, V743I, R753G, N758H, E762G, D1135N,
D1180G, E1219V, Q1221H, Q1256R, H1264Y,
A1320V, A1323D, R1333K
A10T, I322V, S409I, E4270, R654L, K673E, V743I,
R753G, E762G, N869S, N1054D, R1114G, D1135N,
D1180G, E1219V, Q1221H, H1264Y, A1320V,
A1323D, R1333K
A10T, I322V, S409I, E4270, R654L, L727I, V743I,
R753G, E762G, R859S, N946D, F1134L, D1135N,
D1180G, E1219V, Q1221H, H1264Y, N1317T,
A1320V, A1323D, R1333K
A10T, I322V, S409I, E427G, R654L, K673E, V743I,
R753G, E762G, N803S, N869S, Y1016D, G1077D,
RI114G, F1134L, D1135N, DI180G, E1219V, Q1221H,
H1264Y, V1290G, L1318S, A1320V, A1323D,
R1333K
A10T, I322V, S409I, E427G, R654L, K673E, V743I,
R753G, E762G, N803S, N869S, Y1016D, G1077D,
R1114G, F1134L, D1135N, K1151E, D1180G, E1219V,
Q1221H, H1264Y, V1290G, L1318S, A1320V,
R1333K
A10T, I322V, S409I, E427G, R654L, K673E, V743I,
R753G, E762G, N803S, N869S, Y1016D, G1077D,
R1114G, F1134L, D1135N, D1180G, E1219V, Q1221H,
H1264Y, V1290G, L1318S, A1320V, A1323D,
R1333K
A10T, I322V, S409I, E427G, R654L, K673E, F693L,
V743I, R753G, E762G, N803S, N869S, L921P, Y1016D,
G1077D, F1080S, R1114G, D1135N, D1180G, E1219V,
Q1221H, H1264Y, L1318S, A1320V, A1323D,
R1333K
A10T, I322V, S409I, E427G, E630K, R654L, K673E,
V743I, R753G, E762G, Q768H, N803S, N869S, Y1016D,
G1077D, R1114G, F1134L, D1135N, D1180G, E1219V,
Q1221H, H1264Y, L1318S, A1320V, R1333K
A10T, I322V, S409I, E427G, R654L, K673E, F693L,
V743I, R753G, E762G, Q768H, N803S, N869S, Y1016D,
G1077D, R1114G, F1134L, D1135N, D1180G, E1219V,
Q1221H, G1223S, H1264Y, L1318S, A1320V,
R1333K
A10T, I322V, S409I, E427G, R654L, K673E, F693L,
V743I, R753G, E762G, N803S, N869S, L921P, Y1016D,
G1077D, F1801S, R1114G, D1135N, D1180G, E1219V,
Q1221H, H1264Y, L1318S, A1320V, A1323D,
R1333K
A10T, I322V, S409I, E427G, R654L, V743I, R753G,
M1021T, D1135N, D1180G, K1211R, E1219V, Q1221H,
H1264Y, A1320V, R1333K
A10T, I322V, S409I, E427G, R654L, K673E, V743I,
R753G, E762G, M673I, N803S, N869S, G1077D, R1114G,
D1135N, V1139A, D1180G, E1219V, Q1221H,
A1320V, R1333K
A10T, I322V, S409I, E427G, R654L, K673E, V743I,
R753G, E762G, N8038, N869S, R1114G, D1135N,
E1219V, Q1221H, A1320V, R1333K

In some embodiments, the Cas9 protein comprises an amino acid sequence that is at least 80% identical to the amino acid sequence of a Cas9 protein as provided by any one of the variants of Table 1. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 1.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 2. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 2 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100.000-fold, at least 500,000-fold, or at least 1,000,000-fold increased as compared to the activity of Streptococcus pyogenes as provided by SEQ ID NO: 2 on the same target sequence. In some embodiments, the 3′ end of the target sequence is directly adjacent to an AAA, GAA, CAA, or TAA sequence. In some embodiments, the Cas9 protein comprises a combination of mutations that exhibit activity on a target sequence comprising a 5′-NAC-3′ PAM sequence at its 3′-end. In some embodiments, the combination of mutations are present in any one of the clones listed in Table 2. In some embodiments, the combination of mutations are conservative mutations of the clones listed in Table 2. In some embodiments, the Cas9 protein comprises the combination of mutations of any one of the Cas9 clones listed in Table 2.

TABLE 2

NAC PAM Clones
Mutations from wild-type SpCas9 (e.g., SEQ ID NO: 2)
T472I, R753G, K890E, D1332N, R1335Q, T1337N
I1057S, D1135N, P1301S. R1335Q, T1337N
T472I, R753G, D1332N, R1335Q, T1337N
D1135N, E1219V, D1332N, R1335Q, T1337N
T472I, R753G, K890E, D1332N, R1335Q, T1337N
I1057S, D1135N, P1301S, R1335Q, T1337N
T472I, R753G, D1332N, R1335Q, T1337N
T472I, R753G, Q771H, D1332N, R1335Q, T1337N
E627K, T638P, K652T, R753G, N803S, K959N, R1114G,
D1135N, E1219V, D1332N, R1335Q, T1337N
E627K, T638P, K652T, R753G, N803S, K959N,
R1114G, D1135N, K1156E, E1219V, D1332N, R1335Q,
T1337N
E627K, T638P, V647I, R753G, N803S, K959N, G1030R,
I1055E, R1114G, D1135N, E1219V, D1332N,
R1335Q, T1337N
E627K, E630G, T638P, V647A, G687R, N767D, N803S,
K959N, R1114G, D1135N, E1219V, D1332G, R1335Q,
T1337N
E627K, T638P, R753G, N803S, K959N, R1114G,
D1135N, E1219V, N1266H, D1332N, R1335Q, T1337N
E627K, T638P, R753G, N803S, K959N, I1057T,
R1114G, D1135N, E1219V, D1332N, R1335Q, T1337N
E627K, T638P, R753G, N803S, K959N, R1114G,
D1135N, E1219V, D1332N, R1335Q, T1337N
E627K, M631I, T638P, R753G, N803S, K959N, Y1036H,
R1114G, D1135N, E1219V, D1251G, D1332G,
R1335Q, T1337N
E627K, T638P, R753G, N803S, V875I, K959N, Y1016C,
R1114G, D1135N, E1219V, D1251G, D1332G,
R1335Q, T1337N, I1348V
K608R, E627K, T638P, V647I, R654L, R7530, N803S,
T804A, K848N, V922A, K959N, R1114G, D1135N,
E1219V, D1332N, R1335Q, T1337N
K608R, E627K, T638P, V647I, R753G, N803S, V922A,
K959N, K1014N, V1015A, R1114G, D1135N, K1156N,
E1219V, N1252D, D1332N, R1335Q, T1337N
K608R, E627K, R629G, T638P, V647I, A711T, R753G,
K775R, K789E, N803S, K959N, V1015A, Y1036H, R1114G,
D1135N, E1219V, N1286H, D1332N, R1335Q, T1337N
K608R, E627K, T638P, V647I, T740A, R753G, N803S,
K948E, K959N, Y1016S, R1114G, D1135N, E1219V,
N1286H, D1332N, R1335Q, T1337N
K608R, E627K, T638P, V647I, T740A, N803S, K948E,
K959N, Y1016S, R1114G, D1135N, E1219V, N1286H,
D1332N, R1335Q, T1337N
I670S, K608R, E627K, E630G, T638P, V647I, R653K,
R753G, I795L, K797N, N803S, K866R, K890N, K959N,
Y1016C, R1114G, D1135N, E1219V, D1332N, R1335Q, T1337N
K608R, E627K, T638P, V647I, T740A, G752R, R753G,
K797N, N803S, K948E, K959N, V1015A, Y1016S,
R1114G, D1135N, E1219V, N1266H, D1332N, R1335Q, T1337N
I570T, A589V, K608R, E627K, T638P, V647I, R654L,
Q716R, R753G, N803S, K948E, K959N, Y1016S,
R1114G, D1135N, E1207G, E1219V, N1234D,
D1332N, R1335Q, T1337N
K608R, E627K, R629G, T638P, V647I, R654L, Q740R,
R753G, N803S, K959N, N990S, T995S, V1015A,
Y1036D, R1114G, D1135N, E1207G, E1219V,
N1234D, N1266H, D1332N, R1335Q, T1337N
I562F, V565D, I570T, K608R, L625S, E627K, T638P,
V647I, R654I, G752R, R753G, N803S, N808D, K959N,
M1021L, R1114G, D1135N, N1177S, N1234D,
D1332N, R1335Q, T1337N
I562F, I570T, K608R, E627K, T638P, V647I, R753G,
E790A, N803S, K959N, V1015A, Y1036H, R1114G,
D1135N, DI180E, A1184T, E1219V, D1332N, R1335Q, T1337N
I570T, K608R, E627K, T638P, V647I, R654H, R753G,
E790A, N803S, K959N, V1015A, R1114G, D1127A,
D1135N, E1219V, D1332N, R1335Q, T1337N
I570T, K608R, L625S, E627K, T638P, V647I, R654I,
T703P, R753G, N803S, N808D, K959N, M1021L,
R1114G, D1135N, E1219V, D1332N, R1335Q, T1337N
I570S, K608R, E627K, E630G, T638P, V647I, R653K,
R7530, I795L, N803S, K866R, K890N, K959N, Y1016C,
R1114G, D1135N, E1219V, D1332N, R1335Q, T1337N
I570T, K608R, E627K, T638P, V647I, R654H, R753G,
E790A, N803S, K959N, V1016A, R1114G, D1135N,
E1219V, K1246E, D1332N, R1335Q, T1337N
K608R, E627K, T638P, V647I, R654L, K673E, R753G,
E790A, N803S, K948E, K959N, R1114G, D1127G, D1135N,
D1180E, E1219V, N1286H, D1332N, R1335Q, T1337N
K608R, L625S, E627K, T638P, V647I, R654I, I670T,
R753G, N803S, N808D, K959N, M1021L, R1114G,
D1135N, E1219V, N1286H, D1332N, R1335Q, T1337N
E627K, M631V, T638P, V647I, K710E, R753G, N803S,
N808D, K948E, M1021L, R1114G, D1135N, E1219V,
D1332N, R1335Q, T1337N, S1338T, H1349R

In some embodiments, the Cas9 protein comprises an amino acid sequence that is at least 80% identical to the amino acid sequence of a Cas9 protein as provided by any one of the variants of Table 2. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 2.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 2. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 2 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1.000-fold, at least 5,000-fold, at least 10,000-fold, at least 50,000-fold, at least 100.000-fold, at least 500,000-fold, or at least 1,000,000-fold increased as compared to the activity of Streptococcus pyogenes as provided by SEQ ID NO: 2 on the same target sequence. In some embodiments, the 3′ end of the target sequence is directly adjacent to an AAC, GAC, CAC, or TAC sequence.

In some embodiments, the Cas9 protein comprises a combination of mutations that exhibit activity on a target sequence comprising a 5′-NAT-3′ PAM sequence at its 3′-end. In some embodiments, the combination of mutations are present in any one of the clones listed in Table 3. In some embodiments, the combination of mutations are conservative mutations of the clones listed in Table 3. In some embodiments, the Cas9 protein comprises the combination of mutations of any one of the Cas9 clones listed in Table 3.

TABLE 3

NAT PAM Clones
Mutations from wild-type SpCas9 (e.g., SEQ ID NO: 2)
K961E, H985Y, D1135N, K1191N, E1219V,
Q1221H, A1320A, P1321S, R1335L
D1135N, G1218S, E1219V, Q1221H,
P1249S, P1321S, D1322G, R1335L
V743I, R753G, E790A, D1135N, G1218S, E1219V,
Q1221H, A1227V, P1249S, N1286K, A1293T, P1321S,
D1322G, R1335L, T1339I
F575S, M631L, R654L, V748I, V743I, R753G, D853E,
V922A, R1114G D1135N, G1218S, E1219V, Q1221H,
A1227V, P1249S, N1286K, A1293T,
P1321S, D1322G, R1335L, T1339I
F575S, M631L, R654L, R664K, R753G, D853E,
V922A, R1114G D1135N, D1180G, G1218S, E1219V,
Q1221H, P1249S, N1286K, P1321S, D1322G, R1335L
M631L, R654L, R753G, K797E, D853E, V922A,
D1012A, R1114G D1135N, G1218S, E1219V, Q1221H,
P1249S, N1317K, P1321S, D1322G, R1335L
F575S, M631L, R654L, R664K, R753G, D853E,
V922A, R1114G, Y1131C, D1135N, D1180G, G1218S,
E1219V, Q1221H, P1249S, P1321S, D1322G, R1335L
F575S, M631L, R654L, R664K, R753G, D853E,
V922A, R1114G, Y1131C, D1135N, DI180G, G1218S,
E1219V, Q1221H, P1249S, P1321S, DI322G, R1335L
F575S, D596Y, M631L, R654L, R664K, R753G, D853E,
V922A, R1114G, Y1131C, D1135N, D1180G, G1218S,
E1219V, Q1221H, P1249S, Q1256R, P1321S, D1322G, R1335L
F575S, M631L, R654L, R664K, K710E, V750A, R753G,
D853E, V922A, R1114G, Y1131C, D1135N, D1180G,
G1218S, E1219V, Q1221H, P1249S, P1321S, D1322G, R1335L
F575S, M631L, K649R, R654L, R664K, R753G, D853E,
V922A, R1114G, Y1131C, D1135N, K1156E, D1180G,
G1218S, E1219V, Q1221H, P1249S, P1321S, D1322G, R1335L
F575S, M631L, R654L, R664K, R753G, D853E,
V922A, R1114G, Y1131C, D1135N, DI180G, G1218S,
E1219V, Q1221H, P1249S, P1321S, DI322G, R1335L
F575S, M631L, R654L, R664K, R753G, D853E,
V922A, I1057G, R1114G, Y1131C, D1135N, D1180G,
G1218S, E1219V, Q1221H, P1249S,
N1308D, P1321S, D1322G, R1335L
M631L, R654L, R753G, D853E, V922A, R1114G,
Y1131C, D1135N, E1150V, D1180G, G1218S, E1219V,
Q1221H, P1249S, P1321S, D1332G, R1335L
M631L, R654L, R664K, R753G, D853E, I1057V,
Y1131C, D1135N, D1180G, G1218S, E1219V, Q1221H,
P1249S, P1321S, D1332G, R1335L
M631L, R654L, R664K, R753G, I1057V, R1114G, Y1131C,
D1135N, D1180G, G1218S, E1219V, Q1221H,
P1249S, P1321S, D1332G, R1335L

The above description of various napDNAbps which can be used in connection with the prime editors is not meant to be limiting in any way. The prime editors may comprise the canonical SpCas9, or any ortholog Cas9 protein, or any variant Cas9 protein-including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The prime editors utilized in the methods and compositions described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also contain various modifications that alter/enhance their PAM specificities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a references SpCas9 canonical sequences or a reference Cas9 equivalent (e.g., Cas12a/Cpf1). In a particular embodiment, the Cas9 variant having expanded PAM capabilities is SpCas9 (H840A) VRQR (SEQ ID NO: 64), which has the following amino acid sequence (with the V, R, Q. R substitutions relative to the SpCas9 (H840A) of SEQ ID NO: 33 being show in bold underline. In addition, the methionine residue in SpCas9 (H840) was removed for SpCas9 (H840A) VRQR):

(SEQ ID NO: 64)
DKKYSIGLDIGTNSVGWAVITDEYKVPSKKFKVLGNTDRHSIKKNLIGALLFDSGETAEATRLKRTARRRY
TRRKNRICYLQEIFSNEMAKVDDSFFHRLEESELVEEDKKHERHPIFGNIVDEVAYHEKYPTIYHLRKKLVD
STDKADLRLIYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFIERMTNEDKNLPNE
KVLPKHSLLYEYFTVYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEERLKTYAHLFDDKVM
KQLKRRRYTGWGRLSRKLINGIRDKQSGKTILDELKSDGFANRNFMQLIHDDSLTFKEDIQKAQVSGQGDS
LHEHIANLAGSPAIKKGILQTVKVVDELVKVMGRHKPENIVIEMARENQTTQKGQKNSRERMKRIEEGIKE
LGSQILKEHPVENTQLQNEKLYLYYLQNGRDMYVDQELDINRLSDYDVDAIVPQSFLKDDSIDNKVLTRSD
KNRGKSDNVPSEEVVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLANGEIRKRPLIETNGETGEIVW
DKGRDFATVRKVLSMPQVNIVKKTEVQTGGFSKESILPKRNSDKLIARKKDWDPKKYGGFVSPTVAYSVL
VVAKVEKGKSKKLKSVKELLGITIMERSSFEKNPIDFLEAKGYKEVKKDLIIKLPKYSLFELENGRKRMLAS
ARELQKGNELALPSKYVNFLYLASHYEKLKGSPEDNEQKQLFVEQHKHYLDEIIEQISEFSKRVILADANLD
KVLSAYNKHRDKPIREQAENIIHLFTLTNLGAPAAFKYFDTTIDRKQYRSTKEVLDATLIHQSITGLYETRID
LSQLGGD

In another particular embodiment, the Cas9 variant having expanded PAM capabilities is SpCas9 (H840A) VRER, which has the following amino acid sequence (with the V, R, E, R substitutions relative to the SpCas9 (H840A) of SEQ ID NO: 51 being shown in bold underline. In addition, the methionine residue in SpCas9 (H840) was removed for SpCas9 (H840A) VRER):

(SEQ ID NO: 65)
DKKYSIGLDIGTNSVGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYHEKYPTIYHLRKKLVD
STDKADLRLIYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFIERMTNFDKNLPNE
KVLPKHSLLYEYFTVYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEERLKTYAHLEDDKVM
KQLKRRRYTGWGRLSRKLINGIRDKQSGKTILDELKSDGFANRNFMQLIHDDSLTFKEDIQKAQVSGQGDS
LHEHIANLAGSPAIKKGILQTVKVVDELVKVMQRHKPENIVIEMARENQTTQKGQKNSRERMKRIEEGIKE
LGSQILKEHPVENTQLQNEKLYLYYLQNGRDMYVDQELDINRLSDYDVDAIVPQSFLKDDSIDNKVLTRSD
KNRGKSDNVPSEEVVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLANGEIRKRPLIETNGETGEIVW
DKGRDFATVRKVLSMPQVNIVKKTEVQTGGFSKESILPKRNSDKLIARKKDWDPKKYGGFVSPTVAYSVL
VVAKVEKGKSKKLKSVKELLGITIMERSSFEKNPIDFLEAKGYKEVKKDLIIKLPKYSLFELENGRKRMLAS
ARELQKGNELALPSKYVNFLYLASHYEKLKGSPEDNEQKQLFVEQHKHYLDEHIEQISEFSKRVILADANLD
KVLSAYNKHRDKPIREQAENIIHLFTLTNLGAPAAFKYFDTTIDRKEYRSTKEVLDATLIHQSITGLYETRID
LSQLGGD

In some embodiments, the napDNAbp that functions with a non-canonical PAM sequence is an Argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 July; 34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference.

In some embodiments, the napDNAbp is a prokaryotic homolog of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol Direct. 2009 Aug. 25; 4:29. doi: 10.1186/1745-6150-4-29, the entire contents of which is hereby incorporated by reference. In some embodiments, the napDNAbp is a Marinitoga piezophila Argonaute (MpAgo) protein. The CRISPR-associated Marinitoga piezophila Argonaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12:113(15):4057-62, the entire contents of which are hereby incorporated by reference). It should be appreciated that other argonaute proteins may be used, and are within the scope of this disclosure.

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editing fusion proteins provided herein may need to be placed at a precise location, for example where a target base is placed within a 4 base region (e.g., a “editing window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the fusion proteins provided herein may contain a Cas9 domain that is capable of binding a nucleotide sequence that does not contain a canonical (e.g., NGG) PAM sequence, Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015): the entire contents of each are hereby incorporated by reference.

For example, a napDNAbp domain with altered PAM specificity, such as a domain with at least 80%, at least 85%, at least 90%, at least 95%, or at least 99% sequence identity with wild type Francisella novicida Cpf1 (D917, E1006, and D1255) (SEQ ID NO: 66), which has the following amino acid sequence:

(SEQ ID NO: 66)
MSIYQEFVNKYSLSKTLRFELIPQGKTLENIKARGLILDDEKRAKDYKKAKQUIDKYHQFFIEEILSSVCISED
LLQNYSDVYFKLKKSDDDNLQKDFKSAKDTIKKQISEYIKDSEKFKNLFNQNLIDAKKGQESDLILWLKQS
KDNGIELFKANSDITDIDEALEIIKSFKGWTTYFKGFHENRKNVYSSNDIPTSIIYRIVDDNLPKFLENKAKYE
SLKDKAPEAINYEQIKKDLAEELTFDIDYKTSEVNQRVFSLDEVFEIANFNNYLNQSGITKENTIIGGKFVNG
ENTKRKGINEYINLYSQQINDKTLKKYKMSVLFKQILSDTESKSFVIDKLEDDSDVVTTMQSFYEQIAAFKT
VEEKSIKETLSLLFDDLKAQKLDLSKIYFKNDKSLTDLSQQVEDDYSVIGTAVLEYITQQIAPKNLDNPSKKE
QELIAKKTEKAKYLSLETIKLALEEFNKHRDIDKQCRFEEILANFAAIPMIFDEIAQNKDNLAQISIKYQNQG
KKDLLQASAEDDVKAIKDLLDQTNNLLHKLKIFHISQSEDKANILDKDEHFYLVFEECYFELANIVPLYNKI
RNYITQKPYSDEKFKLNFENSTLANGWDKNKEPDNTAILFIKDDKYYLGVMNKKNNKIFDDKAIKENKGE
GYKKIVYKLLPGANKMLPKVFFSAKSIKFYNPSEDILRIRNHSTHTKNGSPQKGYEKFEFNIEDCRKFIDFYK
QSISKHPEWKDFGFRFSDTQRYNSIDEFYREVENQGYKLTFENISESYIDSVVNQGKLYLFQIYNKDFSAYSK
GRPNLHTLYWKALFDERNLQDVVYKLNGEAELFYRKQSIPKKITHPAKEAIANKNKDNPKKESVFEYDLIK
DKRFTEDKFFFHCPITINFKSSGANKENDEINLLLKEKANDVHILSIDRGERHLAYYTLVDGKGNIIKQDTENI
IGNDRMKTNYHDKLAAIEKDRDSARKDWKKINNIKEMKEGYLSQVVHEIAKLVIEYNAIVVFEDLNFGFK
RGRFKVEKQVYQKLEKMLIEKLNYLVFKDNEFDKTGGVLRAYQLTAPFETFKKMGKQTGIIYYVPAGFTS
KICPVTGFVNQLYPKYESVSKSQEFFSKFDKICYNLDKGYFEFSFDYKNFGDKAAKGKWTIASFGSRLINFR
NSDKNHNWDTREVYPTKELEKLLKDYSIEYGHGECIKAAICGESDKKFFAKLTSVLNTILQMRNSKTGTEL
DYLISPVADVNGNFFDSRQAPKNMPQDADANGAYHIGLKGLMLLGRIKNNQEGKKLNLVIKNEEYFEFVQ
NRNN

An additional napDNAbp domain with altered PAM specificity, such as a domain having at least 80%, at least 85%, at least 90%, at least 95%, or at least 99% sequence identity with wild type Geobacillus thermodenitrificans Cas9 (SEQ ID NO: 20), which has the following amino acid sequence:

(SEQ ID NO: 20)
MKYKIGLDIGITSIGWAVINLDIPRIEDLGVRIFDRAENPKTGESLALPRRLARSARRRLRRRKHRLERIRRLF
VREGILTKEELNKLFEKKHEIDVWQLRVEALDRKLNNDELARILLHLAKRRGFRSNRKSERTNKENSTMLK
HIEENQSILSSYRTVAEMVVKDPKESLHKRNKEDNYTNTVARDDLEREIKLIFAKQREYGNIVCTEAFEHEY
ISIWASQRPFASKDDIEKKVGFCTFEPKEKRAPKATYTFQSFTVWEHINKLRLVSPGGIRALTDDERRLIYKQ
AFHKNKITFHDVRTLLNLPDDTRFKGLLYDRNTTLKENEKVRFLELGAYHKIRKAIDSVYGKGAAKSFRPI
DFDTFGYALTMFKDDTDIRSYLRNEYEQNGKRMENLADKVYDEELIEELLNLSFSKFGHLSLKALRNILPY
MEQGEVYSTACERAGYTFTGPKKKQKTVLLPNIPPIANPVVMRALTQARKVVNAIIKKYGSPVSIHIELARE
LSQSFDERRKMQKEQEGNRKKNETAIRQLVEYGLTLNPTGLDIVKFKLWSEQNGKCAYSLQPIEIERLLEPG
YTEVDHVIPYSRSLDDSYTNKVLVLTKENREKGNRTPAEYLGLGSERWQQFETFVLINKQFSKKKRDRLL
RLHYDENEENEFKNRNLNDTRYISRFLANFIREHLKFADSDDKQKVYTVNGRITAHLRSRWNENKNREESN
LHHAVDAAIVACTTPSDIARVTAFYQRREQNKELSKKTDPQFPQPWPHFADELQARLSKNPKESIKALNLG
NYDNEKLESLQPVFVSRMPKRSITGAAHQETLRRYIGIDERSGKIQTVVKKKLSEIQLDKTGHFPMYGKESD
PRTYEAIRQRLLEHNNDPKKAFQEPLYKPKKNGELGPUIRTIKIIDTTNQVIPLNDGKTVAYNSNIVRVDVFE
KDGKYYCVPIYTIDMMKGILPNKAIEPNKPYSEWKEMTEDYTFRESLYPNDLIRIEFPREKTIKTAVGEEIKI
KDLFAYYQTIDSSNGGLSLVSHDNNFSLRSIGSRTLKRFEKYQVDVLGNIYKVRGEKRVGVASSSHSKAGE
TIRPL

In some embodiments, the nucleic acid programmable DNA binding protein (napDNAbp) is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 34(7): 768-73 (2016), PubMed PMID: 27136078; Swarts et al., Nature, 507(7491): 258-61 (2014); and Swarts et al., Nucleic Acids Res. 43(10) (2015): 5120-9, each of which is incorporated herein by reference. The sequence of Natronobacterium gregoryi Argonaute is provided in SEQ ID NO: 67.

The disclosed fusion proteins may comprise a napDNAbp domain having at least 80%, at least 85%, at least 90%, at least 95%, or at least 99% sequence identity with wild type Natronobacterium gregoryi Argonaute (SEQ ID NO: 67), which has the following amino acid sequence:

(SEQ ID NO: 67)
MTVIDLDSTTTADELTSGHTYDISVTLTGVYDNTDEQHPRMSLAFEQDNGERRYITLWKNTTPKDVFTYDY
ATGSTYIFTNIDYEVKDGYENLTATYQTTVENATAQEVGTTDEDETFAGGEPLDHHLDDALNETPDDAETE
SDSGHVMTSFASRDQLPEWTLHTYTLTATDGAKTDTEYARRTLAYTVRQELYTDHDAAPVATDGLMLLT
PEPLGETPLDLDCGVRVEADETRTLDYTTAKDRLLARELVEEGLKRSLWDDYLVRGIDEVLSKEPVLTCDE
FDLHERYDLSVEVGHSGRAYLHINFRHRFVPKLTLADIDDDNIYPGLRVKTTYRPRRGHIVWGLRDECATD
SLNTLGNQSVVAYHRNNQTPINTDLLDAIEAADRRVVETRRQGHGDDAVSFPQELLAVEPNTHQIKQFASD
GFHQQARSKTRLSASRCSEKAQAFAERLDPVRLNGSTVEFSSEFFTGNNEQQLRLLYENGESVLTFRDGAR
GAHPDETFSKGIVNPPESFEVAVVLPEQQADTCKAQWDTMADLLNQAGAPPTRSETVQYDAFSSPESISLN
VAGAIDPSEVDAAFVVLPPDQEGFADLASPTETYDELKKALANMGIYSQMAYFDRFRDAKIFYTRNVALG
LLAAAGGVAFITEHAMPGDADMFIGIDVSRSYPEDGASGQINIAATATAVYKDGTILGHSSTRPQLGEKLQ
STDVRDIMKNAILGYQQVTGESPTHIVIHRDGFMNEDLDPATEFLNEQGVEYDIVEIRKQPQTRLLAVSDVQ
YDTPVKSIAAINQNEPRATVATFGAPEYLATRDGGGLPRPIQIERVAGETDIETLTRQVYLLSQSHIQVHNST
ARLPITTAYADQASTHATKGYLVQTGAFESNVGFL

In addition, any available methods may be utilized to obtain or construct a variant or mutant Cas9 protein. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual ⁽⁴th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Because of their nature, gain-of-function mutations are usually dominant.

Mutations can be introduced into a reference Cas9 protein using site-directed mutagenesis. Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation. More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Mutations may also be introduced by directed evolution processes, such as phage-assisted continuous evolution (PACE) or phage-assisted noncontinuous evolution (PANCE). The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference. Variant Cas9s may also be obtain by phage-assisted non-continuous evolution (PANCE),” which as used herein, refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

Any of the references noted above which relate to Cas9 or Cas9 equivalents are hereby incorporated by reference in their entireties, if not already stated so.

### Other Programmable Nucleases

In various embodiments described herein, the prime editors comprise a napDNAbp, such as a Cas9 protein. These proteins are “programmable” by way of their becoming complexed with a guide RNA (or a PEgRNA, as the case may be), which guides the Cas9 protein to a target site on the DNA which possess a sequence that is complementary to the spacer portion of the gRNA (or PEgRNA) and also which possesses the required PAM sequence. However, in certain embodiment envisioned here, the napDNAbp may be substituted with a different type of programmable protein, such as a zinc finger nuclease or a transcription activator-like effector nuclease (TALEN).

It is contemplated that suitable nucleases do not necessarily need to be “programmed” by a nucleic acid targeting molecule (such as a guide RNA), but rather, may be programmed by defining the specificity of a DNA-binding domain, such as and in particular, a nuclease. Just as in prime editing with napDNAbp moieties, it is preferable that such alternative programmable nucleases be modified such that only one strand of a target DNA is cut. In other words, the programmable nucleases should function as nickases, preferably. Once a programmable nuclease is selected (e.g., a ZFN or a TALEN), then additional functionalities may be engineered into the system to allow it to operate in accordance with a prime editing-like mechanism. For example, the programmable nucleases may be modified by coupling (e.g., via a chemical linker) an RNA or DNA extension arm thereto, wherein the extension arm comprises a primer binding site (PBS) and a DNA synthesis template. The programmable nuclease may also be coupled (e.g., via a chemical or amino acid linker) to a polymerase, the nature of which will depend upon whether the extension arm is DNA or RNA. In the case of an RNA extension arm, the polymerase can be an RNA-dependent DNA polymerase (e.g., reverse transcriptase). In the case of a DNA extension arm, the polymerase can be a DNA-dependent DNA polymerase (e.g., a prokaryotic polymerase, including Pol I, Pol II, or Pol III, or a eukaryotic polymerase, including Pol a, Pol b, Pol g, Pol d, Pol e, or Pol z). The system may also include other functionalities added as fusions to the programmable nucleases, or added in trans to facilitate the reaction as a whole (e.g., (a) a helicase to unwind the DNA at the cut site to make the cut strand with the 3′ end available as a primer, (b) a FEN1 to help remove the endogenous strand on the cut strand to drive the reaction towards replacement of the endogenous strand with the synthesized strand, or (c) a nCas9:gRNA complex to create a second site nick on the opposite strand, which may help drive the integration of the synthesize repair through favored cellular repair of the non-edited strand). In an analogous manner to prime editing with a napDNAbp, such a complex with an otherwise programmable nuclease could be used to synthesize and then install a newly synthesized replacement strand of DNA carrying an edit of interest permanently into a target site of DNA.

Suitable alternative programmable nucleases are well known in the art which may be used in place of a napDNAbp:gRNA complex to construct an alternative prime editor system that can be programmed to selectively bind a target site of DNA, and which can be further modified in the manner described above to co-localize a polymerase and an RNA or DNA extension arm comprising a primer binding site and a DNA synthesis template to specific nick site. For example, Transcription Activator-Like Effector Nucleases (TALENs) may be used as the programmable nuclease in the prime editing methods and compositions of matter described herein. TALENS are artificial restriction enzymes generated by fusing the TAL effector DNA binding domain to a DNA cleavage domain. These reagents enable efficient, programmable, and specific DNA cleavage and represent powerful tools for genome editing in situ. Transcription activator-like effectors (TALEs) can be quickly engineered to bind practically any DNA sequence. The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety. In addition, TALENS are described in WO 2015/027134, U.S. Pat. No. 9,181,535, Boch et al., “Breaking the Code of DNA Binding Specificity of TAL-Type 11 Effectors”, Science, vol. 326. pp. 1509-1512 (2009), Bogdanove et al., TAL Effectors: Customizable Proteins for DNA Targeting, Science, vol. 333, pp. 1843-1846 (2011), Cade et al., “Highly efficient generation of heritable zebrafish gene mutations using homo- and heterodimeric TALENs”, Nucleic Acids Research, vol. 40, pp. 8001-8010 (2012), and Cermak et al., “Efficient design and assembly of custom TALEN and other TAL effector-based constructs for DNA targeting”. Nucleic Acids Research, vol. 39, No. 17, e82 (2011), each of which are incorporated herein by reference.

Zinc finger nucleases may also be used as alternative programmable nucleases for use in the prime editing methods and compositions disclosed herein in place of napDNAbps, such as Cas9 nickases. Like with TALENS, the ZFN proteins may be modified such that they function as nickases, i.e., engineering the ZFN such that it cleaves only one strand of the target DNA in a manner similar to the napDNAbp used with the prime editors in the methods and compositions described herein. ZFN proteins have been extensively described in the art, for example, in Carroll et al., “Genome Engineering with Zinc-Finger Nucleases,” Genetics, August 2011, Vol. 188: 773-782; Durai et al., “Zinc finger nucleases: custom-designed molecular scissors for genome engineering of plant and mammalian cells,” Nucleic Acids Res, 2005, Vol. 33: 5978-90; and Gaj et al., “ZFN, TALEN, and CRISPR/Cas-based methods for genome engineering,” Trends Biotechnol. 2013, Vol. 31: 397-405, each of which are incorporated herein by reference in their entireties.

### Prime Editors: Polymerase Domain (e.g., Reverse Transcriptases)

The present disclosure provides compositions and methods for prime editing with improved editing efficiency and/or reduced indel formation by inhibiting the DNA mismatch repair pathway while conducting prime editing of a target site. Accordingly, the present disclosure provides a method for editing a nucleic acid molecule by prime editing that involves contacting a nucleic acid molecule with a prime editor, a pegRNA, and an inhibitor of the DNA mismatch repair pathway, thereby installing one or more modifications to the nucleic acid molecule at a target site with increased editing efficiency and/or lower indel formation. The present disclosure further provides polynucleotides for editing a DNA target site by prime editing comprising a nucleic acid sequence encoding a napDNAbp, a polymerase, and an inhibitor of the DNA mismatch repair pathway, wherein the napDNAbp and polymerase is capable in the presence of a pegRNA of installing one or more modifications in the DNA target site with increased editing efficiency and/or lower indel formation. Thus, the methods and compositions described herein utilize prime editors, which may comprise a polymerase (e.g., a reverse transcriptase).

In various embodiments, the prime editors used in the methods and compositions disclosed herein includes a polymerase (e.g., DNA-dependent DNA polymerase or RNA-dependent DNA polymerase, such as, reverse transcriptase), or a variant thereof, which can be provided as a fusion protein with a napDNAbp or other programmable nuclease, or provide in trans.

Any polymerase may be used in the prime editors with the methods and compositions disclosed herein. The polymerases may be wild type polymerases, functional fragments, mutants, variants, or truncated variants, and the like. The polymerases may include wild type polymerases from eukaryotic, prokaryotic, archael, or viral organisms, and/or the polymerases may be modified by genetic engineering, mutagenesis, directed evolution-based processes. The polymerases may include T7 DNA polymerase. T5 DNA polymerase, T4 DNA polymerase. Klenow fragment DNA polymerase, DNA polymerase III and the like. The polymerases may also be thermostable, and may include Taq, Tne, Tma, Pfu, Tfl, Tth, Stoffel fragment, VENT® and DEEPVENT® DNA polymerases, KOD, Tgo, JDF3, and mutants, variants and derivatives thereof (see U.S. Pat. Nos. 5,436,149; 4,889,818; 4,965,185; 5,079,352; 5,614,365; 5,374,553; 5,270,179; 5,047,342; 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J.-M, et al., Nuc. Acids Res. 22(15):3259-3260 (1994), each of which are incorporated by reference). For synthesis of longer nucleic acid molecules (e.g., nucleic acid molecules longer than about 3-5 Kb in length), at least two DNA polymerases can be employed. In certain embodiments, one of the polymerases can be substantially lacking a 3′ exonuclease activity and the other may have a 3′ exonuclease activity. Such pairings may include polymerases that are the same or different. Examples of DNA polymerases substantially lacking in 3′ exonuclease activity include, but are not limited to. Taq, Tne(exo-), Tma(exo-), Pfu(exo-), Pwo(exo-), exo-KOD and Tth DNA polymerases, and mutants, variants and derivatives thereof.

Preferably, the polymerase usable in the prime editors utilized in the methods and compositions disclosed herein are “template-dependent” polymerase (since the polymerases are intended to rely on the DNA synthesis template to specify the sequence of the DNA strand under synthesis during prime editing. As used herein, the term “template DNA molecule” refers to that strand of a nucleic acid from which a complementary nucleic acid strand is synthesized by a DNA polymerase, for example, in a primer extension reaction of the DNA synthesis template of a PEgRNA.

As used herein, the term “template dependent manner” is intended to refer to a process that involves the template dependent extension of a primer molecule (e.g., DNA synthesis by DNA polymerase). The term “template dependent manner” refers to polynucleotide synthesis of RNA or DNA wherein the sequence of the newly synthesized strand of polynucleotide is dictated by the well-known rules of complementary base pairing (see, for example, Watson, J. D. el al., In: Molecular Biology of the Gene, ⁴th Ed., W. A. Benjamin. Inc., Menlo Park. Calif. (1987)). The term “complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide. As such, in the case of prime editing, it can be said that the single strand of DNA synthesized by the polymerase of the prime editor against the DNA synthesis template is said to be “complementary” to the sequence of the DNA synthesis template.

### A. Exemplary Polymerases

In various embodiments, the prime editors utilized in the methods and compositions described herein comprise a polymerase. The disclosure contemplates any wild type polymerase obtained from any naturally-occurring organism or virus, or obtained from a commercial or non-commercial source. In addition, the polymerases usable in the prime editors can include any naturally-occurring mutant polymerase, engineered mutant polymerase, or other variant polymerase, including truncated variants that retain function. The polymerases usable herein may also be engineered to contain specific amino acid substitutions, such as those specifically disclosed herein. In certain preferred embodiments, the polymerases usable in the prime editors utilized in the methods and compositions of the present disclosure are template-based polymerases, i.e., they synthesize nucleotide sequences in a template-dependent manner.

A polymerase is an enzyme that synthesizes a nucleotide strand and which may be used in connection with the prime editor systems utilized in the methods and compositions described herein. The polymerases are preferably “template-dependent” polymerases (i.e., a polymerase which synthesizes a nucleotide strand based on the order of nucleotide bases of a template strand). In certain configurations, the polymerases can also be a “template-independent” (i.e., a polymerase which synthesizes a nucleotide strand without the requirement of a template strand). A polymerase may also be further categorized as a “DNA polymerase” or an “RNA polymerase.” In various embodiments, the prime editor systems comprise a DNA polymerase. In various embodiments, the DNA polymerase can be a “DNA-dependent DNA polymerase” (i.e., whereby the template molecule is a strand of DNA). In such cases, the DNA template molecule can be a PEgRNA, wherein the extension arm comprises a strand of DNA. In such cases, the PEgRNA may be referred to as a chimeric or hybrid PEgRNA which comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the PEgRNA is RNA, i.e., including an RNA extension. The term “polymerase” may also refer to an enzyme that catalyzes the polymerization of nucleotide (i.e., the polymerase activity). Generally, the enzyme will initiate synthesis at the 3′-end of a primer annealed to a polynucleotide template sequence (e.g., such as a primer sequence annealed to the primer binding site of a PEgRNA), and will proceed toward the 5′ end of the template strand. A “DNA polymerase” catalyzes the polymerization of deoxynucleotides. As used herein in reference to a DNA polymerase, the term DNA polymerase includes a “functional fragment thereof”. A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

In some embodiments, the polymerases can be from bacteriophage. Bacteriophage DNA polymerases are generally devoid of 5′ to 3′ exonuclease activity, as this activity is encoded by a separate polypeptide. Examples of suitable DNA polymerases are T4, T7, and phi29 DNA polymerase. The enzymes available commercially are: T4 (available from many sources e.g., Epicentre) and T7 (available from many sources, e.g., Epicentre for unmodified and USB for 3′ to 5′ exo T7 “Sequenase” DNA polymerase).

The other embodiments, the polymerases are archaeal polymerases. There are 2 different classes of DNA polymerases which have been identified in archaea: 1. Family B/pol I type (homologs of Pfu from Pyrococcus furiosus) and 2. pol II type (homologs of P. furiosus DP1/DP2 2-subunit polymerase). DNA polymerases from both classes have been shown to naturally lack an associated 5′ to 3′ exonuclease activity and to possess 3′ to 5′ exonuclease (proofreading) activity. Suitable DNA polymerases (pol I or pol II) can be derived from archaea with optimal growth temperatures that are similar to the desired assay temperatures. Thermostable archaeal DNA polymerases are isolated from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. There are 3 classes of eubacterial DNA polymerases, pol I, II, and III. Enzymes in the Pol I DNA polymerase family possess 5′ to 3′ exonuclease activity, and certain members also exhibit 3′ to 5′ exonuclease activity. Pol II DNA polymerases naturally lack 5′ to 3′ exonuclease activity, but do exhibit 3′ to 5′ exonuclease activity. Pol III DNA polymerases represent the major replicative DNA polymerase of the cell and are composed of multiple subunits. The pol III catalytic subunit lacks 5′ to ‘3′ exonuclease activity, but in some cases’ 3′ to 5′ exonuclease activity is located in the same polypeptide. There are a variety of commercially available Pol I DNA polymerases, some of which have been modified to reduce or abolish 5′ to ‘3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritina (Tma UITma). Additional eubacteria related to those listed above are described in Thermophilic Bacteria (Kristjansson, J. K., ed.) CRC Press, Inc., Boca Raton, Fla., 1992.

The invention further provides for chimeric or non-chimeric DNA polymerases that are chemically modified according to methods disclosed in U.S. Pat. Nos. 5,677,152, 6,479,264 and 6,183,998, the contents of which are hereby incorporated by reference in their entirety. Additional archaea DNA polymerases related to those listed above are described in the following references: Archaea: A Laboratory Manual (Robb, F. T. and Place. A. R., eds.), Cold Spring Harbor Laboratory Press, Cold Spring Harbor, N.Y., 1995 and Thermophilic Bacteria (Kristjansson, J. K., ed.) CRC Press, Inc., Boca Raton, Fla., 1992.

### B. Exemplary Reverse Transcriptases

In various embodiments, the prime editors utilized in the methods and compositions described herein comprise a reverse transcriptase as the polymerase. The disclosure contemplates any wild type reverse transcriptase obtained from any naturally-occurring organism or virus, or obtained from a commercial or non-commercial source. In addition, the reverse transcriptases usable in the prime editors utilized in the methods and compositions of the disclosure can include any naturally-occurring mutant RT, engineered mutant RT, or other variant RT, including truncated variants that retain function. The RTs may also be engineered to contain specific amino acid substitutions, such as those specifically disclosed herein.

Reverse transcriptases are multi-functional enzymes typically with three enzymatic activities including RNA- and DNA-dependent DNA polymerization activity, and an RNaseH activity that catalyzes the cleavage of RNA in RNA-DNA hybrids. Some mutants of reverse transcriptases have disabled the RNaseH moiety to prevent unintended damage to the mRNA. These enzymes that synthesize complementary DNA (cDNA) using mRNA as a template were first identified in RNA viruses. Subsequently, reverse transcriptases were isolated and purified directly from virus particles, cells or tissues. (e.g., see Kacian et al., 1971, Biochim. Biophys. Acta 46: 365-83; Yang et al., 1972, Biochem. Biophys. Res. Comm. 47: 505-11; Gerard et al., 1975, J. Virol. 15: 785-97; Liu et al., 1977, Arch. Virol. 55 187-200; Kato et al., 1984, J. Virol. Methods 9: 325-39; Luke et al., 1990. Biochem. 29: 1764-69 and Le Grice et al., 1991. J. Virol. 65: 7004-07, each of which are incorporated by reference). More recently, mutants and fusion proteins have been created in the quest for improved properties such as thermostability, fidelity and activity. Any of the wild type, variant, and/or mutant forms of reverse transcriptase which are known in the art or which can be made using methods known in the art are contemplated herein.

The reverse transcriptase (RT) gene (or the genetic information contained therein) can be obtained from a number of different sources. For instance, the gene may be obtained from eukaryotic cells which are infected with retrovirus, or from a number of plasmids which contain either a portion of or the entire retrovirus genome. In addition, messenger RNA-like RNA which contains the RT gene can be obtained from retroviruses. Examples of sources for RT include, but are not limited to, Moloney murine leukemia virus (M-MLV or MLVRT); human T-cell leukemia virus type 1 (HTLV-1); bovine leukemia virus (BLV); Rous Sarcoma Virus (RSV); human immunodeficiency virus (HIV); yeast, including Saccharomyces, Neurospora, Drosophila; primates; and rodents. See, for example, Weiss, et al., U.S. Pat. No. 4,663,290 (1987); Gerard, G. R., DNA:271-79 (1986); Kotewicz, M. L., et al., Gene 35:249-58 (1985); Tanese, N., et al., Proc. Natl. Acad. Sci. (USA):4944-48 (1985); Roth, M. J., at al., J. Biol. Chem. 260:9326-35 (1985); Michel, F., et al., Nature 316:641-43 (1985); Akins, R. A., et al., Cell 47:505-16 (1986), EMBO J. 4:1267-75 (1985); and Fawcett, D. F., Cell 47:1007-15 (1986) (each of which are incorporated herein by reference in their entireties).

### Wild Type RTs

Exemplary enzymes for use with the prime editors can include, but are not limited to, M-MLV reverse transcriptase and RSV reverse transcriptase. Enzymes having reverse transcriptase activity are commercially available. In certain embodiments, the reverse transcriptase provided in trans to the other components of the prime editor system. That is, the reverse transcriptase is expressed or otherwise provided as an individual component. i.e., not as a fusion protein with a napDNAbp.

A person of ordinary skill in the art will recognize that wild type reverse transcriptases, including but not limited to, Moloney Murine Leukemia Virus (M-MLV); Human Immunodeficiency Virus (HIV) reverse transcriptase and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase may be suitably used in the subject methods and composition described herein.

Exemplary wild type RT enzymes are as follows:

| Description | Sequence |
|---|---|
| REVERSE | TLNIEDEYRLHETSKEPDVSLGSTWLSDFPQAWAETGGMGLAVRQAPLIIPLKATST |
| TRANSCRIPTASE | PVSIKQYPMSQEARLGIKPHIQRLLDQGILVPCQSPWNTPLLPVKKPGTNDYRPVQD |
| (M-MLV RT) WILD | LREVNKRVEDIHPTVPNPYNLLSGLPPSHQWYTVLDLKDAFFCLRLHPTSQPLFAFE |
| TYPE | WRDPEMGISGQLTWTRLPQGFKNSPTLFDEALHRDLADFRIQHPDLILLQYVDDLL |
| MOLONEY | LAATSELDCQQGTRALLQTLGNLGYRASAKKAQICQKQVKYLGYLLKEGQRWLT |
| MURINE | EARKETVMGQPTPKTPRQLREFLGTAGFCRLWIPGFAEMAAPLYPLIKTGTLFNW |
| LEUKEMIA VIRUS | GPDQQKAYQEIKQALLTAPALGLPDLTKPFELFVDEKQGYAKGVLTQKLGPWRRP |
| USED IN PE1 | VAYLSKKLDPVAAGWPPCLRMVAAIAVLTKDAGKLTMGQPLVILAPHAVEALVK |
| (PRIME EDITOR 1 | QPPDRWLSNARMTHYQALLLDTDRVQFGPVVALNPATLLPLPEEGLQHNCLDILA |
| FUSION PROTEIN | EAHGTRPDLTDQPLPDADHTWYTDGSSLLQEGQRKAGAAVTTETEVIWAKALPAG |
| DISCLOSED | TSAQRAELIALTQALKMAEGKKLNVYTDSRYAFATAHIHGEIYRRRGLLTSEGKEI |
| HEREIN) | KNKDEILALLKALFLPKRLSIIHCPGHQKGHSAEARGNRMADQAARKAAITETPDT |
| | STLLIENSSP (SEQ ID NO: 81) |
| REVERSE | AFPLERPDWDYTTQAGRNHLVHYRQLLLAGLQNAGRSPINLAKVKGITQGPNESP |
| TRANSCRIPTASE | SAFLERLKEAYRRYTPYDPEDPGQETNVSMSFIWQSAPDIGRKLGRLEDLKSKTLG |
| MOLONEY | DLVREAEKIFNKRETPEEREERIRRETEEKEERRRTVDEQKEKERDRRRHREMSKLL |
| MURINE | ATVVIGQEQDRQEGERKRPQLDKDQCAYCKEKGHWAKDCPKKPRGPRGPRPQTS |
| LEUKEMIA VIRUS | LLTLGDXGGQGQDPPPEPRITLKVGGQPVTFLVDTGAQHSVLTQNPGPLSDKSAW |
| REF SEQ. | VQGATGGKRYRWTTDRKVHLATGKVTHSFLHVPDCPYPLLGRDLLTKLKAQIHFE |
| AAA66622.1 | GSGAQVVGPMGQPLQVLTLNIEDEYRLHETSKEPDVSLGFTWLSDFPQAWAESGG |
| | MGLAVRQAPLIIPLKATSTPVSIKQYPMSQEARLGIKPHIQRLLDQGILVPCQSPWNT |
| | PLLPVKKPGTNDYRPVQDLREVNKRVEDIHPTVPNPYNLLSGLPPSHQWYTVLDLK |
| | DAFFCLRLHPTSQPLFAFEWRDPEMGISGQLTWTRLPQGFKNSPTLFDEALHRDLA |
| | DFR (SEQ ID NO: 69) |
| REVERSE | TLQLEEEYRLFEPESTQKQEMDIWLKNFPQAWAETGGMGTAHCQAPVLIQLKATA |
| TRANSCRIPTASE | TPISIRQYPMPHEAYQGIKPHIRRMLDQGILKPCQSPWNTPLLPVKKPGTEDYRPVQ |
| FELINE LEUKEMIA | DLREVNKRVEDIHPTVPNPYNLLSTLPPSHPWYTVLDLKDAFFCLRLHSESQLLEAF |
| VIRUS | EWRDPEIGLSGQLTWTRLPQGFKNSPTLFDEALHSDLADFRVRYPALVLLQYVDDL |
| REF SEQ. | LLAAATRTECLEGTKALLETLGNKGYRASAKKAQICLQEVTYLGYSLKDGQRWLT |
| NP955579.1 | KARKEAILSIPVPKNSRQVREFLGTAGYCRLWIPGFAELAAPLYPLTRPGTLFQWGT |
| | EQQLAFEDIKKALLSSPALGLPDITKPFELFIDENSGFAKGVLVQKLGPWKRPVAYL |
| | SKKLDTVASGWPPCLRMVAAIAILVKDAGKLTLGQPLTILTSHPVEALVRQPPNKW |
| | LSNARMTHYQAMLLDAERVHFGPTVSLNPATLLPLPSGGNHHDCLQILAETHGTRP |
| | DLTDQPLPDADLTWYTDGSSFIRNGEREAGAAVTTESEVIWAAPLPPGTSAQRAELI |
| | ALTQALKMAEGKKLTVYTDSRYAFATTHVHGEIYRRRGLLTSEGKEIKNKNEILAL |
| | LEALFLPKRLSIIHCPGHQKGDSPQAKGNRLADDTAKKAATETHSSLTVL |
| | SEQ ID NO: 70) |
| REVERSE | PISPIETVPVKLKPGMDGPKVKQWPLTEEKIKALVEICTEMEKEGKISKIGPENPYNT |
| TRANSCRIPTASE | PVFAIKKKDSTKWRKLVDFRELNKRTQDFWEVQLGIPHPAGLKKKKSVTVLDVGD |
| HIV-1 RT, CHAIN A | AYFSVPLDEDFRKYTAFTIPSINNETPGIRYQYNVLPQGWKGSPAIFQSSMTKILEPF |
| REF SEQ. ITL3-A | RKQNPDIVIYQYMDDLYVGSDLEIGQHRTKIEELRQHLLRWGLTTPDKKHQKEPPF |
| | LWMGYELHPDKWTVQPIVLPEKDSWTVNDIQKLVGKLNWASQIYPGIKVRQLXK |
| | LLRGTKALTEVIPLTEEAELELAENREILKEPVHGVYYDPSKDLIAEIQKQGQGQWT |
| | YQIYQEPFKNLKTGKYARMRGAHTNDVKQLTEAVQKITTESIVIWGKTPKFKLPIQ |
| | KETWETWWTEYWQATWIPEWEFVNTPPLVKLWYQLEKEPIVGAETFYVDGAANR |
| | ETKLGKAGYVINRGRQKVVTLTDTTNQKTELQAIYLALQDSGLEVNIVTDSQYAL |
| | GIIQAQPDQSESELVNQIIEQLIKKEKVYLAWVPAHKGIGGNEQVDKLVSAGIRKV |
| | (SEQ ID NO: 71) |
| | See Martinelli et al., Virology, 1990, 174(1): 135-144, which |
| | is incorporated by reference |
| REVERSE | PISPIETVPVKLKPGMDGPKVKQWPLTEEKIKALVEICTEMEKEGKISKIGPENPYNT |
| TRANSCRIPTASE | PVFAIKKKDSTKWRKLVDFRELNKRTQDFWEVQLGIPHPAGLKKKKSVTVLDVGD |
| HIV-1 RT, CHAIN B | AYFSVPLDEDFRKYTAFTIPSINNETPGIRYQYNVLPQGWKGSPAIFQSSMTKILEPF |
| REF SEQ. ITL3-B | RKQNPDIVIYQYMDDLYVGSDLEIGQHRTKIEELRQHLLRWGLTTPDKKHQKEPPF |
| | LWMGYELHPDKWTVQPIVLPEKDSWTVNDIQKLVGKLNWASQIYPGIKVRQLCKL |
| | LRGTKALTEVIPLTEEAELELAENREILKEPVHGVYYDPSKDLIAEIQKQGQGQWTY |
| | QIYQEPFKNLKTGKYARMRGAHTNDVKQLTEAVQKITTESIVIWGKTPKFKLPIQK |
| | ETWETWWTEYWQATWIPEWEFVNTPPLVKLWYQLEKEPIVGAETF (SEQ ID NO: 72) |
| | See Stammers et al., J. Mol. Biol., 1994, 242(4): 586-588, which |
| | is incorporated by reference |
| REVERSE | TVALHLAIPLKWKPNHTPVWIDQWPLPEGKLVALTQLVEKELQLGHIEPSLSCWNT |
| TRANSCRIPTASE | PVFVIRKASGSYRLLHDLRAVNAKLVPFGAVQQGAPVLSALPRGWPLMVLDLKDC |
| ROUS SARCOMA | FFSIPLAEQDREAFAFTLPSVNNQAPARRFQWKVLPQGMTCSPTICQLIVGQILEPLR |
| VIRUS RT | LKHPSLRMLHYMDDLLLAASSHDGLEAAGEEVISTLERAGFTISPDKVQKEPGVQY |
| REF SEQ. ACL14945 | LGYKLGSTYAAPVGLVAEPRIATLWDVQKLVGSLQWLRPALGIPPRLRGPFYEQLR |
| | GSDPNEAREWNLDMKMAWREIVQLSTTAALERWDPALPLEGAVARCEQGAIGVL |
| | GQGLSTHPRPCLWLFSTQPTKAFTAWLEVLTLLITKLRASAVRTFGKEVDILLLPAC |
| | FRDELPLPEGILLALRGFAGKIRSSDTPSIFDIARPLHVSLKVRVTDHPVPGPTVFTDA |
| | SSSTHKGVVVWREGPRWEIKEIADLGASVQQLEARAVAMALLLWPTTPTNVVTDS |
| | AFVAKMLLKMGQEGVPSTAAAFILEDALSQRSAMAAVLHVRSHSEVPGFFTEGND |
| | VADSQATFQAYPLREAKDLHTALHIGPRALSKACNISMQQAREVVQTCPHCNSAP |
| | ALEAGVNPRGLGPLQIWQTDFTLEPRMAPRSWLAVTVDTASSAIVVTQHGRVTSV |
| | AAQHHWATVIAVLGRPKAIKTDNGSCFTSKSTREWLARWGIAHTTGIPGNSQGQA |
| | MVERANRLLKDKIRVLAEGDGFMKRIPTSKQGELLAKAMYALNHFERGENTKTPI |
| | QKHWRPTVLTEGPPVKIRIETGEWEKGWNVLVWGRGYAAVKNRDTDKVIWVPSR |
| | KVKPDIAQKDEVTKKDEASPLFA (SEQ ID NO: 73) |
| | See Yasukawa et al., J. Biochem. 2009, 145(3): 315-324, which |
| | is incorporated by reference |
| REVERSE | MMDHLLQKTQIQNQTEQVMNITNPNSIYIKGRLYFKGYKKIELHCFVDTGASLCIA |
| TRANSCRIPTASE | SKFVIPEEHWINAERPIMVKIADGSSITINKVCRDIDLIIAGEIFHIPTVYQQESGIDFII |
| CAULIFLOWER | GNNFCQLYEPFIQFTDRVIFTKDRTYPVHIAKLTRAVRVGTEGFLESMKKRSKTQQP |
| MOSAIC VIRUS RT | EPVNISTNKIAILSEGRRLSEEKLFITQQRMQKIEELLEKVCSENPLDPNKTKQWMK |
| REF SEQ. | ASIKLSDPSKAIKVKPMKYSPMDREEFDKQIKELLDLKVIKPSKSPHMAPAFLVNNE |
| AGT42196 | AEKRRGKKRMVVNYKAMNKATVGDAYNLPNKDELLTLIRGKKIFSSFDCKSGFW |
| | QVLLDQDSRPLTAFTCPQGHYEWNVVPFGLKQAPSIFQRHMDEAFRVFRKFCCVY |
| | VDDILVFSNNEEDHLLHVAMILQKCNQHGIILSKKKAQLFKKKINFLGLEIDEGTHK |
| | PQGHILEHINKFPDTLEDKKQLQRFLGILTYASDYIPKLAQIRKPLQAKLKENVPWK |
| | WTKEDTLYMQKVKKNLQGFPPLHHPLPEEKLIIETDASDDYWGGMLKAIKINEGT |
| | NTELICRYASGSFKAAEKNYHSNDKETLAVINTIKKFSIYLTPVHFLIRTDNTHFKSF |
| | VNLNYKGDSKLGRNIRWQAWLSHYSFDVEHIKGTDNHFADELSREFNRVNS(SEQ |
| | ID NO: 74) |
| | See Farzadfar et al., Virus Genes, 2013, 47(2): 347-356, which |
| | is incorporated by reference |
| REVERSE | MKEKISKIDKNFYTDIFIKTSFQNEFEAGGVIPPIAKNQVSTISNKNKTFYSLAHSSPH |
| TRANSCRIPTASE | YSIQTRIEKELLKNIPLSASSFAFRKERSYLHYLEPHTQNVKYCHLDIVSFFHSIDVNI |
| KLEBSIELLA | VRDTFSVYFSDEFLVKEKQSLLDAFMASVTLTAELDGVEKTFIPMGFKSSPSISNIIF |
| PNEUMONIA | RKIDILIQKFCDKNKITYTRYADDLLFSTKKENNILSSTFFINEISSILSINKFKLNKSK |
| REF SEQ. | YLYKEGTISLGGYVIENILKDNSSGNIRLSSSKLNPLYKALYEIKKGSSSKHICIKVEN |
| RFF81513.1 | LKLKRFIYKKNKEKFEAKFYSSQLKNKLLGYRSYLLSFVIFHKKYKCINPIFLEKCVF |
| | LISEIESIMNRKF (SEQ ID NO: 75) |
| REVERSE | MKITSNNVTAVINGKGWHSINWKKCHQHVKTIQTRIAKAACNQQWRTVGRLQRL |
| TRANSCRIPTASE | LVRSFSARALAVKRVTENSGRKTPGVDGQIWSTPESKWEAIFKLRRKGYKPLPLKR |
| ESCHERICHIA | VFIPKSNGKKRPLGIPVMLDRAMQALHLLGLEPVSETNADHNSYGFRPARCTADAI |
| COLI RT | QQVCNMYSSRNASKWVLEGDIKGCFEHISHEWLLENIPMDKQILRNWLKAGIIEKS |
| REF SEQ. | IFSKTLSGTPQGGIISPVLANMALDGLERLLQNRFGRNRLI (SEQ ID NO: 76) |
| TGH57013 | |
| REVERSE | MSKIKINYEKYHIKPFPHFDQRIKVNKKVKENLQNPFYIAAHSFYPFIHYKKISYKFK |
| TRANSCRIPTASE | NGTLSSPKERDIFYSGHMDGYIYKHYGEILNHKYNNTCIGKGIDHVSLAYRNNKMG |
| BACILLUS | KSNIHFAAEVINFISEQQQAFIFVSDFSSYFDSLDHAILKEKLIEVLEEQDKLSKDWW |
| SUBTILIS RT | NVFKHITRYNWVEKEEVISDLECTKEKIARDKKSRERYYTPAEFREFRKRVNIKSND |
| REF SEQ. QBJ66766 | TGVGIPQGTAISAVLANVYAIDLDQKLNQYALKYGGIYRRYSDDIIMVLPMTSDGQ |
| | DPSNDHVSFIKSVVKRNKVTMGDSKTSVLYYANNNIYEDYQRKRESKMDYLGFSF |
| | DGMTVKIREKSLFKYYHRTYKKINSINWASVKKEKKVGRKKLYLLYSHLGRNYKG |
| | HGNFISYCKKAHAVFEGNKKIESLINQQIKRHWKKIQKRLVDV (SEQ ID NO: 77) |
| EUBACTERIUM | DTSNLMEQILSSDNLNRAYLQVVRNKGAEGVDGMKYTELKEHLAKNGETIKGQL |
| RECTALE GROUP | RTRKYKPQPARRVEIPKPDGGVRNLGVPTVTDRFIQQAIAQVLTPIYEEQFHDHSYG |
| II INTRON RT | FRPNRCAQQAILTALNIMNDGNDWIVDIDLEKFFDTVNHDKLMTLIGRTIKDGDVIS |
| | IVRKYLVSGIMIDDEYEDSIVGTPQGGNLSPLLANIMLNELDKEMEKRGLNFVRYA |
| | DDCIIMVGSEMSANRVMRNISRFIEEKLGLKVNMTKSKVDRPSGLKYLGFGFYFDP |
| | RAHQFKAKPHAKSVAKFKKRMKELTCRSWGVSNSYKVEKLNQLIRGWINYFKIGS |
| | MKTLCKELDSRIRYRLRMCIWKQWKTPQNQEKNLVKLGIDRNTARRVAYTGKRIA |
| | YVCNKGAVNVAISNKRLASFGLISMLDYYIEKCVTC (SEQ ID NO: 78) |
| GEOBACILLUS | ALLERILARDNLITALKRVEANQGAPGIDGVSTDQLRDYIRAHWSTIHAQLLAGTY |
| STEAROTHERMOPHILUS | RPAPVRRVEIPKPGGGTRQLGIPTVVDRLIQQAILQELTPIFDPDFSSSSFGFRPGRNA |
| GROUP II | HDAVRQAQGYIQEGYRYVVDMDLEKFFDRVNHDILMSRVARKVKDKRVLKLIRA |
| INTRON RT | YLQAGVMIEGVKVQTEEGTPQGQPLSPLLANILLDDLDKELEKRGLKFCRYADDC |
| | NIYVKSLRAGQRVKQSIQRFLEKTLKLKVNEEKSAVDRPWKRAFLGFSFTPERKAR |
| | IRLAPRSIQRLKQRIRQLTNPNWSISMPERIHRVNQYVMGWIGYFRLVETPSVLQTIE |
| | GWIRRRLRLCQWLQWKRVRTRIRELRALGLKETAVMEIANTRKGAWRTTKTPQL |
| | HQALGKTYWTAQGLKSLTQR (SEQ ID NO: 79) |

### Variant and Error-Prone RTs

Reverse transcriptases are essential for synthesizing complementary DNA (cDNA) strands from RNA templates. Reverse transcriptases are enzymes composed of distinct domains that exhibit different biochemical activities. The enzymes catalyze the synthesis of DNA from an RNA template, as follows: In the presence of an annealed primer, reverse transcriptase binds to an RNA template and initiates the polymerization reaction. RNA-dependent DNA polymerase activity synthesizes the complementary DNA (cDNA) strand, incorporating dNTPs. RNase H activity degrades the RNA template of the DNA:RNA complex. Thus, reverse transcriptases comprise (a) a binding activity that recognizes and binds to a RNA/DNA hybrid, (b) an RNA-dependent DNA polymerase activity, and (c) an RNase H activity. In addition, reverse transcriptases generally are regarded as having various attributes, including their thermostability, processivity (rate of dNTP incorporation), and fidelity (or error-rate). The reverse transcriptase variants contemplated herein may include any mutations to reverse transcriptase that impacts or changes any one or more of these enzymatic activities (e.g., RNA-dependent DNA polymerase activity, RNase H activity, or DNA/RNA hybrid-binding activity) or enzyme properties (e.g., thermostability, processivity, or fidelity). Such variants may be available in the art in the public domain, available commercially, or may be made using known methods of mutagenesis, including directed evolutionary processes (e.g., PACE or PANCE).

In various embodiments, the reverse transcriptase may be a variant reverse transcriptase. As used herein, a “variant reverse transcriptase” includes any naturally occurring or genetically engineered variant comprising one or more mutations (including singular mutations, inversions, deletions, insertions, and rearrangements) relative to a reference sequences (e.g., a reference wild type sequence). An RT may have several activities, including an RNA-dependent DNA polymerase activity, ribonuclease H activity, and DNA-dependent DNA polymerase activity. Collectively, these activities enable the enzyme to convert single-stranded RNA into double-stranded cDNA. In retroviruses and retrotransposons, this cDNA can then integrate into the host genome, from which new RNA copies can be made via host-cell transcription. Variant RT's may comprise a mutation which impacts one or more of these activities (either which reduces or increases these activities, or which eliminates these activities all together). In addition, variant RTs may comprise one or more mutations which render the RT more or less stable, less prone to aggregation, and facilitates purification and/or detection, and/or other the modification of properties or characteristics.

A person of ordinary skill in the art will recognize that variant reverse transcriptases derived from other reverse transcriptases, including but not limited to Moloney Murine Leukemia Virus (M-MLV); Human Immunodeficiency Virus (HIV) reverse transcriptase and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase may be suitably used in the subject methods and composition described herein.

One method of preparing variant RTs is by genetic modification (e.g., by modifying the DNA sequence of a wild-type reverse transcriptase). A number of methods are known in the art that permit the random as well as targeted mutation of DNA sequences (see for example, Ausubel et. al. Short Protocols in Molecular Biology (1995) 3.sup.rd Ed. John Wiley & Sons, Inc.). In addition, there are a number of commercially available kits for site-directed mutagenesis, including both conventional and PCR-based methods. Examples include the QuikChange Site-Directed Mutagenesis Kits (AGILENT®), the Q5® Site-Directed Mutagenesis Kit (NEW ENGLAND BIOLABS®), and GeneArt™ Site-Directed Mutagenesis System (THERMOFISHER SCIENTIFIC®).

In addition, mutant reverse transcriptases may be generated by insertional mutation or truncation (N-terminal, internal, or C-terminal insertions or truncations) according to methodologies known to one skilled in the art. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual ⁽⁴th ed., Cold Spring Harbor Laboratory Press, Cold Spring Harbor. N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Because of their nature, gain-of-function mutations are usually dominant.

Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation.

More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Methods of random mutagenesis, which will result in a panel of mutants bearing one or more randomly situated mutations, exist in the art. Such a panel of mutants may then be screened for those exhibiting the desired properties, for example, increased stability, relative to a wild-type reverse transcriptase.

An example of a method for random mutagenesis is the so-called “error-prone PCR method.” As the name implies, the method amplifies a given sequence under conditions in which the DNA polymerase does not support high fidelity incorporation. Although the conditions encouraging error-prone incorporation for different DNA polymerases vary, one skilled in the art may determine such conditions for a given enzyme. A key variable for many DNA polymerases in the fidelity of amplification is, for example, the type and concentration of divalent metal ion in the buffer. The use of manganese ion and/or variation of the magnesium or manganese ion concentration may therefore be applied to influence the error rate of the polymerase.

In various aspects, the RT of the prime editors may be an “error-prone” reverse transcriptase variant. Error-prone reverse transcriptases that are known and/or available in the art may be used. The error-rate of any particular reverse transcriptase is a property of the enzyme's “fidelity,” which represents the accuracy of template-directed polymerization of DNA against its RNA template. An RT with high fidelity has a low-error rate. Conversely, an RT with low fidelity has a high-error rate. The fidelity of M-MLV-based reverse transcriptases are reported to have an error rate in the range of one error in 15,000 to 27,000 nucleotides synthesized. See Boutabout et al., “DNA synthesis fidelity by the reverse transcriptase of the yeast retrotransposon Ty1,” Nucleic Acids Res, 2001, 29: 2217-2222, which is incorporated by reference. Thus, for purposes of this application, those reverse transcriptases considered to be “error-prone” or which are considered to have an “error-prone fidelity” are those having an error rate that is less than one error in 15,000 nucleotides synthesized.

Error-prone reverse transcriptase also may be created through mutagenesis of a starting RT enzyme (e.g., a wild type M-MLV RT). The method of mutagenesis is not limited and may include directed evolution processes, such as phage-assisted continuous evolution (PACE) or phage-assisted noncontinuous evolution (PANCE). The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; international PCT Application. PCT/US2011/066747, filed Dec. 22, 2011, published as WO 2012/088381 on Jun. 28, 2012; U.S. Application, U.S. Pat. No. 9,023,594, issued May 5, 2015. International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference.

Error-prone reverse transcriptases may also be obtain by phage-assisted non-continuous evolution (PANCE),” which as used herein, refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

Other error-prone reverse transcriptases have been described in the literature, each of which are contemplated for use in the herein methods and compositions. For example, error-prone reverse transcriptases have been described in Bebenek et al., “Error-prone Polymerization by HIV-1 Reverse Transcriptase,” J Biol Chem, 1993. Vol. 268: 10324-10334 and Sebastian-Martin et al., “Transcriptional inaccuracy threshold attenuates differences in RNA-dependent DNA synthesis fidelity between retroviral reverse transcriptases,” Scientific Reports, 2018, Vol. 8: 627, each of which are incorporated by reference. Still further, reverse transcriptases, including error-prone reverse transcriptases can be obtained from a commercial supplier, including ProtoScript® (II) Reverse Transcriptase, AMV Reverse Transcriptase, WarmStart® Reverse Transcriptase, and M-MuLV Reverse Transcriptase, all from NEW ENGLAND BIOLABS®, or AMV Reverse Transcriptase XL, SMARTScribe Reverse Transcriptase, GPR ultra-pure MMLV Reverse Transcriptase, all from TAKARA BIO USA, INC. (formerly CLONTECH).

The herein disclosure also contemplates reverse transcriptases having mutations in RNaseH domain. As mentioned above, one of the intrinsic properties of reverse transcriptases is the RNase H activity, which cleaves the RNA template of the RNA:cDNA hybrid concurrently with polymerization. The RNase H activity can be undesirable for synthesis of long cDNAs because the RNA template may be degraded before completion of full-length reverse transcription. The RNase H activity may also lower reverse transcription efficiency, presumably due to its competition with the polymerase activity of the enzyme. Thus, the present disclosure contemplates any reverse transcriptase variants that comprise a modified RNaseH activity. The herein disclosure also contemplates reverse transcriptases having mutations in the RNA-dependent DNA polymerase domain. As mentioned above, one of the intrinsic properties of reverse transcriptases is the RNA-dependent DNA polymerase activity, which incorporates the nucleobases into the nascent cDNA strand as coded by the template RNA strand of the RNA:cDNA hybrid. The RNA-dependent DNA polymerase activity can be increased or decreased (i.e., in terms of its rate of incorporation) to either increase or decrease the processivity of the enzyme. Thus, the present disclosure contemplates any reverse transcriptase variants that comprise a modified RNA-dependent DNA polymerase activity such that the processivity of the enzyme of either increased or decreased relative to an unmodified version.

Also contemplated herein are reverse transcriptase variants that have altered thermostability characteristics. The ability of a reverse transcriptase to withstand high temperatures is an important aspect of cDNA synthesis. Elevated reaction temperatures help denature RNA with strong secondary structures and/or high GC content, allowing reverse transcriptases to read through the sequence. As a result, reverse transcription at higher temperatures enables full-length cDNA synthesis and higher yields, which can lead to an improved generation of the 3′ flap ssDNA as a result of the prime editing process. Wild type M-MLV reverse transcriptase typically has an optimal temperature in the range of 37-48° C.; however, mutations may be introduced that allow for the reverse transcription activity at higher temperatures of over 48° C., including 49° C., 50° C., 51° C., 52° C., 53° C., 54° C., 55° C., 56° C., 57° C., 58° C., 59° C. 60° C., 61° C. 62° C., 63° C., 64° C., 65° C., 66° C., and higher.

The variant reverse transcriptases contemplated herein, including error-prone RTs, thermostable RTs, increase-processivity RTs, can be engineered by various routine strategies, including mutagenesis or evolutionary processes. In some cases, the variants can be produced by introducing a single mutation. In other cases, the variants may require more than one mutation. For those mutants comprising more than one mutation, the effect of a given mutation may be evaluated by introduction of the identified mutation to the wild-type gene by site-directed mutagenesis in isolation from the other mutations borne by the particular mutant. Screening assays of the single mutant thus produced will then allow the determination of the effect of that mutation alone.

Variant RT enzymes used herein may also include other “RT variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference RT protein, including any wild type RT, or mutant RT, or fragment RT, or other variant of RT disclosed or contemplated herein or known in the art.

In some embodiments, an RT variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT. In some embodiments, the RT variant comprises a fragment of a reference RT, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference RT. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type RT (M-MLV reverse transcriptase) (e.g., SEQ ID NO: 81) or to any of the reverse transcriptases of SEQ ID NOs: 69-79.

In some embodiments, the disclosure also may utilize RT fragments which retain their functionality and which are fragments of any herein disclosed RT proteins. In some embodiments, the RT fragment is at least 100 amino acids in length. In some embodiments, the fragment is at least 100, 150, 200, 250, 300, 350, 400, 450, 500, 550, or up to 600 or more amino acids in length.

In still other embodiments, the disclosure also may utilize RT variants which are truncated at the N-terminus or the C-terminus, or both, by a certain number of amino acids which results in a truncated variant which still retains sufficient polymerase function. In some embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the N-terminal end of the protein. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the C-terminal end of the protein. In still other embodiments, the RT truncated variant has a truncation at the N-terminal and the C-terminal end which are the same or different lengths. For example, the prime editors utilized in the methods and compositions disclosed herein may include a truncated version of M-MLV reverse transcriptase. In this embodiment, the reverse transcriptase contains 4 mutations (D200N, T306K, W313F, T330P; noting that the L603W mutation present in PE2 is no longer present due to the truncation). The DNA sequence encoding this truncated editor is 522 bp smaller than PE2, and therefore makes its potentially useful for applications where delivery of the DNA sequence is challenging due to its size (i.e., adeno-associated virus and lentivirus delivery). This embodiment is referred to as MMLV-RT(trunc) and has the following amino acid sequence:

| | |
|---|---|
| MMLV-RT | TLNIEDEYRLHETSKEPDVSLGSTWLSDFPQAWAETGGMG |
| (TRUNC) | LAVRQAPLIIPLKATSTPVSIKQYPMSQEARLGIKPHIQR |
| | LLDQGILVPCQSPWNTPLLPVKKPGTNDYRPVQDLREVNK |
| | RVEDIHPTVPNPYNLLSGLPPSHQWYTVLDLKDAFFCLRL |
| | HPTSQPLFAFEWRDPEMGISGQLTWTRLPQGFKNSPTLFN |
| | EALHRDLADFRIQHPDLILLQYVDDLLLAATSELDCQQGT |
| | RALLQTLGNLGYRASAKKAQICQKQVKYLGYLLKEGQRWL |
| | TEARKETVMGQPTPKTPRQLREFLGKAGFCRLFIPGFAEM |
| | AAPLYPLTKPGTLFNWGPDQQKAYQEIKQALLTAPALGLP |
| | DLTKPFELFVDEKQGYAKGVLTQKLGPWRRPVAYLSKKLD |
| | PVAAGWPPCLRMVAAIAVLTKDAGKLTMGQPLVILAPHAV |
| | EALVKQPPDRWLSNARMTHYQALLLDTDRVQFGPVVALNP |
| | ATLLPLPEEGLQHNCLDNSRLIN |
| | (SEQ ID NO: 80) |

In various embodiments, the prime editors utilized in the methods and compositions disclosed herein may comprise one of the RT variants described herein, or a RT variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants. In still other embodiments, the present methods and compositions may utilize a DNA polymerase that has been evolved into a reverse transcriptase, as described in Effefson et al., “Synthetic evolutionary origin of a proofreading reverse transcriptase,” Science, Jun. 24, 2016, Vol. 352: 1590-1593, the contents of which are incorporated herein by reference.

In certain other embodiments, the reverse transcriptase is provided as a component of a fusion protein also comprising a napDNAbp. In other words, in some embodiments, the reverse transcriptase is fused to a napDNAbp as a fusion protein.

In various embodiments, variant reverse transcriptases can be engineered from wild type M-MLV reverse transcriptase as represented by SEQ ID NO: 81.

In various embodiments, the prime editors utilized in the methods and compositions described herein (with RT provided as either a fusion partner or in trans) can include a variant RT comprising one or more of the following mutations: P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, or D653N in the wild type M-MLV RT of SEQ ID NO: 81 or at a corresponding amino acid position in another wild type RT polypeptide sequence.

Some exemplary reverse transcriptases that can be fused to napDNAbp proteins or provided as individual proteins according to various embodiments of this disclosure are provided below. Exemplary reverse transcriptases include variants with at least 80%, at least 85%, at least 90%, at least 95%, or at least 99% sequence identity to the following wild-type enzymes or partial enzymes:

| | Sequence (variant substitutions |
|---|---|
| Description | relative to wild type) |
| Reverse | TLNIEDEYRLHETSKEPDVSLGSTWLSDFPQAWAETGGMGLAVRQ |
| transcriptase (M- | APLIPLKATSTPVSIKQYPMSQEARLGIKPHIQRLLDQGILVPCQ |
| MLV RT) wild | SPWNTPLLPVKKPGTNDYRPVQDLREVNKRVEDIHPTVPNPYNLL |
| type | SGLPPSHQWYTVLDLKDAFFCLRLHPTSQPLFAFEWRDPEMGISG |
| Moloney murine | QLTWTRLPQGFKNSPTLFDEALHRDLADFRIQHPDLILLQYVDDL |
| leukemia Virus | LLAATSELDCQQGTRALLQTLGNLGYRASAKKAQICQKQVKYLGY |
| Used in PE1 | LLKEGQRWLTEARKETVMGQPTPKTPRQLREFLGTAGFCRLWIPG |
| (prime editor 1 | FAEMAAPLYPLTKTGTLFNWGPDQQKAYQEIKQALLTAPALGLPD |
| fusion protein | LTKPFELFVDEKQGYAKGVLTQKLGPWRRPVAYLSKKLDPVAAGW |
| disclosed herein) | PPCLRMVAAIAVLTKDAGKLTMGQPLVILAPHAVEALVKQPPDRW |
| | LSNARMTHYQALLLDTDRVQFGPVVALNPATLLPLPEEGLQHNCL |
| | DILAEAHGTRPDLTDQPLPDADHTWYTDGSSLLQEGQRKAGAAVT |
| | TETEVIWAKALPAGTSAQRAELIALTQALKMAEGKKLNVYTDSRY |
| | AFATAHIHGEIYRRRGLLTSEGKEIKNKDEILALLKALFLPKRLS |
| | IIHCPGHQKGHSAEARGNRMADQAARKAAITETPDTSTLLIENSS |
| | P |
| | (SEQ ID NO: 81) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| | QSPWNTPLLPVKKPGTNDYRPVQDLREVNKRVEDIHPTVPNPYNL |
| | LSGLPPSHQWYTVLDLKDAFFCLRLHPTSQPLFAFEWRDPEMGIS |
| | GQLTWTRLPQGFKNSPTLFNEALHRDLADFRIQHPDLILLQYVDD |
| | LLLAATSELDCQQGTRALLQTLGNLGYRASAKKAQICQKQVKYLG |
| | YLLKEGQRWLTEARKETVMGQPTPKTPRQLREFLGTAGFCRLWIP |
| | GFAEMAAPLYPLIKTGTLENWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGLLTSEGKEIKNKDEILALLKALFLPKRL |
| | SIIHCPGHQKGHSAEARGNRMADQAARKAAITETPDTSTLLIENS |
| | SP |
| | (SEQ ID NO: 82) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| | LSGLPPSHQWYTVLDLKDAFFCLRLHPTSQPLFAFEWRDPEMGIS |
| | GQLTWTRLPQGFKNSPTLFNEALHRDLADFRIQHPDLILLQYVDD |
| | LLLAATSELDCQQGTRALLQTLGNLGYRASAKKAQICQKQVKYLG |
| | YLLKEGQRWLTEARKETVMGQPTPKTPRQLREFLGTAGFCRLWIP |
| | GFAEMAAPLYPLTKPGTLFNWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGLLTSEGKEIKNKDEILALLKALFLPKRL |
| | SIIHCPGHQKGHSAEARGNRMADQAARKAAITETPDTSTLLIENS |
| | SP |
| | (SEQ ID NO: 83) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| L603W | LSGLPPSHQWYTVLDLKDAFFCLRLHPTSQPLFAFEWRDPEMGIS |
| | GQLTWTRLPQGFKNSPTLFNEALHRDLADFRIQHPDLILLQYVDD |
| | LLLAATSELDCQQGTRALLQTLGNLGYRASAKKAQICQKQVKYLG |
| | YLLKEGQRWLTEARKETVMGQPTPKTPRQLREFLGTAGFCRLWIP |
| | GFAEMAAPLYPLTKPGTLFNWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGWLTSEGKEIKNKDEILALLKALFLPKRL |
| | SIIHCPGHQKGHSAEARGNRMADQAARKAAITETPDTSTLLIENS |
| | SP |
| | (SEQ ID NO: 84) |
| M-MLV RT | TLNIEDEYRLHETSKEPDVSLGSTWLSDFPQAWAETGGMGLAVRQ |
| D200N | APLIIPLKATSTPVSIKQYPMSQKARLGIKPHIQRLLDQGILVPC |
| T330P | QSPWNTPLLPVKKPGTNDYRPVQDLREVNKRVEDIHPTVPNPYNL |
| L603W | LSGLPPSHQWYTVLDLKDAFFCLRLHPTSQPLFAFEWRDPEMGIS |
| E69K | GQLTWTRLPQGFKNSPTLFNEALHRDLADFRIQHPDLILLQYVDD |
| | LLLAATSELDCQQGTRALLQTLGNLGYRASAKKAQICQKQVKYLG |
| | YLLKEGQRWLTEARKETVMGQPTPKTPRQLREFLGTAGFCRLWIP |
| | GFAEMAAPLYPLTKPGTLFNWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGWLTSEGKEIKNKDEILALLKALFLPKRL |
| | SIIHCPGHQKGHSAEARGNRMADQAARKAAITETPDTSTLLIENS |
| | SP |
| | (SEQ ID NO: 85) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| L603W | LSGLPPSHQWYTVLDLKDAFFCLRLHPTSQPLFAFEWRDPEMGIS |
| E302R | GQLTWTRLPQGFKNSPTLFNEALHRDLADFRIQHPDLILLQYVDD |
| | LLLAATSELDCQQGTRALLQTLGNLGYRASAKKAQICQKQVKYLG |
| | YLLKEGQRWLTEARKETVMGQPTPKTPRQLRRFLGTAGFCRLWIP |
| | GFAEMAAPLYPLTKPGTLFNWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGWLTSEGKEIKNKDEILALLKALFLPKRL |
| | SIIHCPGHQKGHSAEARGNRMADQAARKAAITETPDTSTLLIENS |
| | SP |
| | (SEQ ID NO: 86) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| L603W | LSGLPPSHQWYTVLDLKDAFFCLRLHPTSQPLFAFEWRDPEMGIS |
| E607K | GQLTWTRLPQGFKNSPTLFNEALHRDLADFRIQHPDLILLQYVDD |
| | LLLAATSELDCQQGTRALLQTLGNLGYRASAKKAQICQKQVKYLG |
| | YLLKEGQRWLTEARKETVMGQPTPKTPRQLREFLGTAGFCRLWIP |
| | GFAEMAAPLYPLTKPGTLENWGPDQQKAYQEIKQALLTAPALGLP |
| | DLTKPFELFVDEKQGYAKGVLTQKLQPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGWLTSKGKEIKNKDEILALLKALFLPKRL |
| | SIIHCPGHQKGHSAEARGNRMADQAARKAAITETPDTSTLLIENS |
| | SP |
| | (SEQ ID NO: 87) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| L603W | LSGPPPSHQWYTVLDLKDAFFCLRLHPTSQPLFAFEWRDPEMGIS |
| L139P | GQLTWTRLPQGFKNSPTLFNEALHRDLADFRIQHPDLILLQYVDD |
| | LLLAATSELDCQQGTRALLQTLGNLGYRASAKKAQICQKQVKYLG |
| | YLLKEGQRWLTEARKETVMGQPTPKTPRQLREFLGTAGFCRLWIP |
| | GFAEMAAPLYPLTKPGTLFNWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGWLTSEGKEIKNKDEILALLKALFLPKRL |
| | SIIHCPGHQKGHSAEARGNRMADQAARKAAITETPDTSTLLIENS |
| | SP |
| | (SEQ ID NO: 88) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| L603W | LSGLPPSHQWYTVLDLKDAFFCLRLHPTSQPLFAFEWRDPEMGIS |
| LA35G | GQLTWTRLPQGFKNSPTLFNEALHRDLADFRIQHPDLILLQYVDD |
| | LLLAATSELDCQQGTRALLQTLGNLGYRASAKKAQICQKQVKYLG |
| | YLLKEGQRWLTEARKETVMGQPTPKTPRQLREFLGTAGFCRLWIP |
| | GFAEMAAPLYPLTKPGTLENWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVIGAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGWLTSEGKEIKNKDEILALLKALFLPKRL |
| | SIIHCPGHQKGHSAEARGNRMADQAARKAAITETPDTSTLLIENS |
| | SP |
| | (SEQ ID NO: 89) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| L603W | LSGLPPSHQWYTVLDLKDAFFCLRLHPTSQPLFAFEWRDPEMGIS |
| N454K | GQLTWTRLPQGFKNSPTLFNEALHRDLADFRIQHPDLILLQYVDD |
| | LLLAATSELDCQQGTRALLQTLGNLGYRASAKKAQICQKQVKYLG |
| | YLLKEGQRWLTEARKETVMGQPTPKTPRQLREFLGTAGFCRLWIP |
| | GFAEMAAPLYPLTKPGTLENWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSKARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGWLTSEGKEIKNKDEILALLKALFLPKRL |
| | SIIHCPGHQKGHSAEARGNRMADQAARKAAITETPDTSTLLIENS |
| | SP |
| | (SEQ ID NO: 90) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| L603W | LSGLPPSHQWYTVLDLKDAFFCLRLHPTSQPLFAFEWRDPEMGIS |
| T306K | GQLTWTRLPQGFKNSPTLFNEALHRDLADFRIQHPDLILLQYVDD |
| | LLLAATSELDCQQGTRALLQTLGNLGYRASAKKAQICQKQVKYLG |
| | YLLKEGQRWLTEARKETVMGQPTPKTPRQLREFLGKAGFCRLWIP |
| | GFAEMAAPLYPLTKPGTLFNWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGWLTSEGKEIKNKDEILALLKALFLPKRL |
| | SIIHCPGHQKGHSAEARGNRMADQAARKAAITETPDTSTLLIENS |
| | SP |
| | (SEQ ID NO: 91) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| L603W | LSGLPPSHQWYTVLDLKDAFFCLRLHPTSQPLFAFEWRDPEMGIS |
| W313F | GQLTWTRLPQGFKNSPTLFNEALHRDLADFRIQHPDLILLQYVDD |
| | LLLAATSELDCQQGTRALLQTLGNLGYRASAKKAQICQKQVKYLG |
| | YLLKEGQRWLTEARKETVMGQPTPKTPRQLREFLGTAGFCRLFIP |
| | GFAEMAAPLYPLTKPGTLENWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGWLTSEGKEIKNKDEILALLKALFLPKRL |
| | SIIHCPGHQKGHSAEARGNRMADQAARKAAITETPDTSTLLIENS |
| | SP |
| | (SEQ ID NO: 92) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| L603W | LSGLPPSHQWYTVLDLKDAFFCLRLHPTSQPLFAFEWRDPEMGIS |
| D524G | GQLTWTRLPQGFKNSPTLFNEALHRDLADFRIQHPDLILLQYVDD |
| E562Q | LLLAATSELDCQQGTRALLQTLGNLGYRASAKKAQICQKQVKYLG |
| D583N | YLLKEGQRWLTEARKETVMGQPTPKTPRQLREFLGTAGFCRLWIP |
| | GFAEMAAPLYPLTKPGTLENWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTGGSSLLQEGQRKAGAAV |
| | TTETEVIWAKALPAGTSAQRAQLIALTQALKMAEGKKLNVYTNSR |
| | YAFATAHIHGEIYRRRGWLTSEGKEIKNKDEILALLKALFLPKRL |
| | SIIHCPGHQKGHSAEARGNRMADQAARKAAITETPDTSTLLIENS |
| | SP |
| | (SEQ ID NO: 93) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| L603W | LSGLPPSHQWYTVLDLKDAFFCLRLHPTSQPLFAFEWRDPEMGIS |
| E302R | GQLTWTRLPQGFKNSPTLFNEALHRDLADFRIQHPDLILLQYVDD |
| W313F | LLLAATSELDCQQGTRALLQTLGNLGYRASAKKAQICQKQVKYLG |
| | YLLKEGQRWLTEARKETVMGQPTPKTPRQLRRFLGTAGFCRLFIP |
| | GFAEMAAPLYPLTKPGTLFNWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGWLTSEGKEIKNKDEILALLKALFLPKRL |
| | SIIHCPGHQKGHSAEARGNRMADQAARKAAITETPDTSTLLIENS |
| | SP |
| | (SEQ ID NO: 94) |
| M-MLV RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| L603W | LSGPPPSHQWYTVLDLKDAFFCLRLHPTSQPLFAFEWRDPEMGIS |
| E607K | GQLTWTRLPQGFKNSPTLFNEALHRDLADFRIQHPDLILLQYVDD |
| L139P | LLLAATSELDCQQGTRALLQTLGNLGYRASAKKAQICQKQVKYLG |
| | YLLKEGQRWLTEARKETVMGQPTPKTPRQLREFLGTAGFCRLWIP |
| | GFAEMAAPLYPLTKPGTLFNWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGEIYRRRGWLTSKGKEIKNKDEILALLKALFLPKRL |
| | SIHCPGHQKGHSAEARGNRMADQAARKAAITETPDTSTLLIENSS |
| | P |
| | (SEQ ID NO: 95) |
| M-MLV RT | TLNIEDEYRLHETSKEPDVSLGSTWLSDFPQAWAETGGMGLAVRQ |
| PS1L S67K | APLIILLKATSTPVSIKQYPMKQEARLGIKPHIQRLLDQGILVPC |
| T197A H204R | QSPWNTPLLPVKKPGTNDYRPVQDLREVNKRVEDIHPTVPNPYNL |
| E302K F309N | LSGLPPSHQWYTVLDLKDAFFCLRLHPTSQPLFAFEWRDPEMGIS |
| W313FT330P | GQLTWTRLPQGFKNSPALFDEALRRDLADFRIQHPDLILLQYVDD |
| L435G N454K | LLLAATSELDCQQGTRALLQTLGNLQYRASAKKAQICQKQVKYLG |
| D524G D583N | YLLKEGQRWLTEARKETVMGQPTPKTPRQLRKFLGTAGNCRLFIP |
| H594Q D653N | GFAEMAAPLYPLTKPGTLFNWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVIGAPHAVEALVKQPPDR |
| | WLSKARMTHYQALLLDTDRVQFGPVVALNPATLLPLPEEGLQHNC |
| | LDILAEAHGTRPDLTDQPLPDADHTWYTGGSSLLQEGQRKAGAAV |
| | TTETEVIWAKALPAGTSAQRAELIALTQALKMAEGKKLNVYTNSR |
| | YAFATAHIQGETYRRRGLLTSEGKEIKNKDEILALLKALFLPKRL |
| | SIIHCPGHQKGHSAEARGNRMANQAARKAAITETPDTSTLLIENS |
| | SP |
| | (SEQ ID NO: 96) |
| M-MLV RT | TLNIEDEYRLHETSKEPDVSLGSTWLSDFPQAWAETGGMGLAVRQ |
| D200N P51L | APLIILLKATSTPVSIKQYPMKQEARLGIKPHIQRLLDQGILVPC |
| S67K T197A | QSPWNTPLLPVKKPGTNDYRPVQDLREVNKRVEDIHPTVPNPYNL |
| H204R E302K | LSGLPPSHQWYTVLDLKDAFFCLRLHPTSQPLFAFEWRDPEMGIS |
| F309N W313F | GQLTWTRLPQGFKNSPALFNEALRRDLADFRIQHPDLILLQYVDD |
| T330P L345G | LLLAATSELDCQQGTRALLQTLGNLGYRASAKKAQICQKQVKYLG |
| N454K D524G | YLLKEGQRWLTEARKETVMGQPTPKTPRQLRKFLGTAGNCRLFIP |
| D583N H594Q | GFAEMAAPLYPLTKPGTLFNWGPDQQKAYQEIKQALLTAPALGLP |
| D653N | DLTKPFELFVDEKQGYAKGVLTQKLGPWRRPVAYLSKKLDPVAAG |
| | WPPCLRMVAAIAVLTKDAGKLTMGQPLVIGAPHAVEALVKQPPDR |
| | WLSKARMTHYQALLLDTDRVQFGPVVALNPATLLPLPEEGLQHNC |
| | LDILAEAHGTRPDLTDQPLPDADHTWYTGGSSLLQEGQRKAGAAV |
| | TTETEVIWAKALPAGTSAQRAELIALTQALKMAEGKKLNVYTNSR |
| | YAFATAHIQGETYRRRGLLTSEGKEIKNKDEILALLKALFLPKRL |
| | SIIHCPGHQKGHSAEARGNRMANQAARKAAITETPDTSTLLIENS |
| | SP |
| | (SEQ ID NO: 97) |
| M-MLV RT | TLNIEDEYRLHETSKEPDVSLGSTWLSDFPQAWAETGGMGLAVRQ |
| D200N T330P | APLIPLKATSTPVSIKQYPMSQEARLGIKPHIQRLLDQGILVPCQ |
| L603W T306K | SPWNTPLLPVKKPGTNDYRPVQDLREVNKRVEDIHPTVPNPYNLL |
| W313F | SGLPPSHQWYTVLDLKDAFFCLRLHPTSQPLFAFEWRDPEMGISG |
| in PE2 | QLTWTRLPQGFKNSPTLFNEALHRDLADFRIQHPDLILLQYVDDL |
| | LLAATSELDCQQGTRALLQTLGNLGYRASAKKAQICQKQVKYLGY |
| | LLKEGQRWLTEARKETVMGQPTPKTPRQLREFLGKAGFCRLFIPG |
| | FAEMAAPLYPLTKPGTLFNWGPDQQKAYQEIKQALLTAPALGLPD |
| | LTKPFELFVDEKQGYAKGVLTQKLGPWRRPVAYLSKKLDPVAAGW |
| | PPCLRMVAAIAVLTKDAGKLTMGQPLVILAPHAVEALVKQPPDRW |
| | LSNARMTHYQALLLDTDRVQFGPVVALNPATLLPLPEEGLQHNCL |
| | DILAEAHGTRPDLTDQPLPDADHTWYTDGSSLLQEGQRKAGAAVT |
| | TETEVIWAKALPAGTSAQRAELIALTQALKMAEGKKLNVYTDSRY |
| | AFATAHIHGEIYRRRGWLTSEGKEIKNKDEILALLKALFLPKRLS |
| | IIHCPGHQKGHSAEARGNRMADQAARKAAITETPDTSTLLIENSS |
| | P |
| | (SEQ ID NO: 98) |

In various other embodiments, the prime editors utilized in the methods and compositions described herein (with RT provided as either a fusion partner or in trans) can include a variant RT comprising one or more of the following mutations: P51X, S67X, E69X, L139X, T197X, D200X, H204X, F209X, E302X, T306X, F309X. W313X. T330X, L345X, L435X, N454X. D524X, E562X, D583X, H594X, L603X, E607X, or D653X in the wild type M-MLV RT of SEQ ID NO: 81 or at a corresponding amino acid position in another wild type RT polypeptide sequence, wherein “X” can be any amino acid.

In various other embodiments, the prime editors utilized in the methods and compositions described herein (with RT provided as either a fusion partner or in trans) can include a variant RT comprising a P51X mutation in the wild type M-MLV RT of SEQ ID NO: 81 or at a corresponding amino acid position in another wild type RT polypeptide sequence, wherein “X” can be any amino acid. In certain embodiments, X is L.

In various other embodiments, the prime editors utilized in the methods and compositions described herein (with RT provided as either a fusion partner or in trans) can include a variant RT comprising a S67X mutation in the wild type M-MLV RT of SEQ ID NO: 81 or at a corresponding amino acid position in another wild type RT polypeptide sequence, wherein “X” can be any amino acid. In certain embodiments, X is K.

In various other embodiments, the prime editors utilized in the methods and compositions described herein (with RT provided as either a fusion partner or in trans) can include a variant RT comprising a E69X mutation in the wild type M-MLV RT of SEQ ID NO: 81 or at a corresponding amino acid position in another wild type RT polypeptide sequence, wherein “X” can be any amino acid. In certain embodiments, X is K.

In various other embodiments, the prime editors utilized in the methods and compositions described herein (with RT provided as either a fusion partner or in trans) can include a variant RT comprising a L139X mutation in the wild type M-MLV RT of SEQ ID NO: 81 or at a corresponding amino acid position in another wild type RT polypeptide sequence, wherein “X” can be any amino acid. In certain embodiments, X is P.

In various other embodiments, the prime editors utilized in the methods and compositions described herein (with RT provided as either a fusion partner or in trans) can include a variant RT comprising a T197X mutation in the wild type M-MLV RT of SEQ ID NO: 81 or at a corresponding amino acid position in another wild type RT polypeptide sequence, wherein “X” can be any amino acid. In certain embodiments, X is A.

In various other embodiments, the prime editors utilized in the methods and compositions described herein (with RT provided as either a fusion partner or in trans) can include a variant RT comprising a D200X mutation in the wild type M-MLV RT of SEQ ID NO: 81 or at a corresponding amino acid position in another wild type RT polypeptide sequence, wherein “X” can be any amino acid. In certain embodiments, X is N.

In various other embodiments, the prime editors utilized in the methods and compositions described herein (with RT provided as either a fusion partner or in trans) can include a variant RT comprising a H204X mutation in the wild type M-MLV RT of SEQ ID NO: 81 or at a corresponding amino acid position in another wild type RT polypeptide sequence, wherein “X” can be any amino acid. In certain embodiments, X is R.

In various other embodiments, the prime editors utilized in the methods and compositions described herein (with RT provided as either a fusion partner or in trans) can include a variant RT comprising a F209X mutation in the wild type M-MLV RT of SEQ ID NO: 81 or at a corresponding amino acid position in another wild type RT polypeptide sequence, wherein “X” can be any amino acid. In certain embodiments, X is N.

In various other embodiments, the prime editors utilized in the methods and compositions described herein (with RT provided as either a fusion partner or in trans) can include a variant RT comprising a E302X mutation in the wild type M-MLV RT of SEQ ID NO: 81 or at a corresponding amino acid position in another wild type RT polypeptide sequence, wherein “X” can be any amino acid. In certain embodiments, X is K.

In various other embodiments, the prime editors utilized in the methods and compositions described herein (with RT provided as either a fusion partner or in trans) can include a variant RT comprising a E302X mutation in the wild type M-MLV RT of SEQ ID NO: 81 or at a corresponding amino acid position in another wild type RT polypeptide sequence, wherein “X” can be any amino acid. In certain embodiments, X is R.

In various other embodiments, the prime editors utilized in the methods and compositions described herein (with RT provided as either a fusion partner or in trans) can include a variant RT comprising a T306X mutation in the wild type M-MLV RT of SEQ ID NO: 81 or at a corresponding amino acid position in another wild type RT polypeptide sequence, wherein “X” can be any amino acid. In certain embodiments, X is K.

In various other embodiments, the prime editors utilized in the methods and compositions described herein (with RT provided as either a fusion partner or in trans) can include a variant RT comprising a F309X mutation in the wild type M-MLV RT of SEQ ID NO: 81 or at a corresponding amino acid position in another wild type RT polypeptide sequence, wherein “X” can be any amino acid. In certain embodiments, X is N.

In various other embodiments, the prime editors utilized in the methods and compositions described herein (with RT provided as either a fusion partner or in trans) can include a variant RT comprising a W313X mutation in the wild type M-MLV RT of SEQ ID NO: 81 or at a corresponding amino acid position in another wild type RT polypeptide sequence, wherein “X” can be any amino acid. In certain embodiments, X is F.

In various other embodiments, the prime editors utilized in the methods and compositions described herein (with RT provided as either a fusion partner or in trans) can include a variant RT comprising a T330X mutation in the wild type M-MLV RT of SEQ ID NO: 81 or at a corresponding amino acid position in another wild type RT polypeptide sequence, wherein “X” can be any amino acid. In certain embodiments, X is P.

In various other embodiments, the prime editors utilized in the methods and compositions described herein (with RT provided as either a fusion partner or in trans) can include a variant RT comprising a L345X mutation in the wild type M-MLV RT of SEQ ID NO: 81 or at a corresponding amino acid position in another wild type RT polypeptide sequence, wherein “X” can be any amino acid. In certain embodiments, X is G.

In various other embodiments, the prime editors utilized in the methods and compositions described herein (with RT provided as either a fusion partner or in trans) can include a variant RT comprising a L435X mutation in the wild type M-MLV RT of SEQ ID NO: 81 or at a corresponding amino acid position in another wild type RT polypeptide sequence, wherein “X” can be any amino acid. In certain embodiments, X is G.

In various other embodiments, the prime editors utilized in the methods and compositions described herein (with RT provided as either a fusion partner or in trans) can include a variant RT comprising a N454X mutation in the wild type M-MLV RT of SEQ ID NO: 81 or at a corresponding amino acid position in another wild type RT polypeptide sequence, wherein “X” can be any amino acid. In certain embodiments, X is K.

In various other embodiments, the prime editors utilized in the methods and compositions described herein (with RT provided as either a fusion partner or in trans) can include a variant RT comprising a D524X mutation in the wild type M-MLV RT of SEQ ID NO: 81 or at a corresponding amino acid position in another wild type RT polypeptide sequence, wherein “X” can be any amino acid. In certain embodiments, X is G.

In various other embodiments, the prime editors utilized in the methods and compositions described herein (with RT provided as either a fusion partner or in trans) can include a variant RT comprising a E562X mutation in the wild type M-MLV RT of SEQ ID NO: 81 or at a corresponding amino acid position in another wild type RT polypeptide sequence, wherein “X” can be any amino acid. In certain embodiments, X is Q.

In various other embodiments, the prime editors utilized in the methods and compositions described herein (with RT provided as either a fusion partner or in trans) can include a variant RT comprising a D583X mutation in the wild type M-MLV RT of SEQ ID NO: 81 or at a corresponding amino acid position in another wild type RT polypeptide sequence, wherein “X” can be any amino acid. In certain embodiments, X is N.

In various other embodiments, the prime editors utilized in the methods and compositions described herein (with RT provided as either a fusion partner or in trans) can include a variant RT comprising a H594X mutation in the wild type M-MLV RT of SEQ ID NO: 81 or at a corresponding amino acid position in another wild type RT polypeptide sequence, wherein “X” can be any amino acid. In certain embodiments, X is Q.

In various other embodiments, the prime editors utilized in the methods and compositions described herein (with RT provided as either a fusion partner or in trans) can include a variant RT comprising a L603X mutation in the wild type M-MLV RT of SEQ ID NO: 81 or at a corresponding amino acid position in another wild type RT polypeptide sequence, wherein “X” can be any amino acid. In certain embodiments, X is W.

In various other embodiments, the prime editors utilized in the methods and compositions described herein (with RT provided as either a fusion partner or in trans) can include a variant RT comprising a E607X mutation in the wild type M-MLV RT of SEQ ID NO: 81 or at a corresponding amino acid position in another wild type RT polypeptide sequence, wherein “X” can be any amino acid. In certain embodiments, X is K.

In various other embodiments, the prime editors utilized in the methods and compositions described herein (with RT provided as either a fusion partner or in trans) can include a variant RT comprising a D653X mutation in the wild type M-MLV RT of SEQ ID NO: 81 or at a corresponding amino acid position in another wild type RT polypeptide sequence, wherein “X” can be any amino acid. In certain embodiments, X is N.

Some exemplary reverse transcriptases that can be fused to napDNAbp proteins or provided as individual proteins according to various embodiments of this disclosure are provided below. Exemplary reverse transcriptases include variants with at least 80%, at least 85%, at least 90%, at least 95% or at least 99% sequence identity to the wild-type enzymes or partial enzymes represented by SEQ ID NOs: 81-98.

The prime editor system utilized in the methods and compositions described here contemplates any publicly-available reverse transcriptase described or disclosed in any of the following U.S. patents (each of which are incorporated by reference in their entireties): U.S. Pat. Nos. 10,202,658; 10,189,831; 10,150,955; 9,932,567; 9,783,791; 9,580,698; 9,534,201; and 9,458,484, and any variant thereof that can be made using known methods for installing mutations, or known methods for evolving proteins. The following references describe reverse transcriptases in art. Each of their disclosures are incorporated herein by reference in their entireties.

Herzig, E., Voronin, N., Kucherenko, N. & Hizi, A. A Novel Leu92 Mutant of HIV-1 Reverse Transcriptase with a Selective Deficiency in Strand Transfer Causes a Loss of Viral Replication. J. Virol. 89, 8119-8129 (2015).

Mohr, G. et al. A Reverse Transcriptase-Cas1 Fusion Protein Contains a Cas6 Domain Required for Both CRISPR RNA Biogenesis and RNA Spacer Acquisition. Mol. Cell 72, 700-714.e8 (2018).

Zhao, C., Liu, F. & Pyle, A. M. An ultraprocessive, accurate reverse transcriptase encoded by a metazoan group II intron. RNA 24, 183-195 (2018).

Zimmerly, S. & Wu, L. An Unexplored Diversity of Reverse Transcriptases in Bacteria. Microbiol Spectr 3, MDNA3-0058-2014 (2015).

Ostertag. E. M. & Kazazian Jr, H. H. Biology of Mammalian L1 Retrotransposons. Annual Review of Genetics 35, 501-538 (2001).

Perach, M. & Hizi, A. Catalytic Features of the Recombinant Reverse Transcriptase of Bovine Leukemia Virus Expressed in Bacteria. Virology 259, 176-189 (1999).

Lim, D. et al. Crystal structure of the moloney murine leukemia virus RNase H domain. J. Virol. 80, 8379-8389 (2006).

Zhao, C. & Pyle, A. M. Crystal structures of a group II intron maturase reveal a missing link in spliceosome evolution. Nature Structural & Molecular Biology 23, 558-565 (2016). Griffiths, D. J. Endogenous retroviruses in the human genome sequence. Genome Biol. 2, REVIEWS1017 (2001).

Baranauskas, A. et al. Generation and characterization of new highly thermostable and processive M-MuLV reverse transcriptase variants. Protein Eng Des Sel 25, 657-668 (2012).

Zimmerly, S., Guo, H., Perlman, P. S. & Lambowltz, A. M. Group II intron mobility occurs by target DNA-primed reverse transcription. Cell82, 545-554 (1995).

Feng, Q., Moran, J. V., Kazazian, H. H. & Boeke. J. D. Human L1 retrotransposon encodes a conserved endonuclease required for retrotransposition. Cell 87, 905-916 (1996).

Berkhout, B., Jebbink. M. & Zsfros, J. Identification of an Active Reverse Transcriptase Enzyme Encoded by a Human Endogenous HERV-K Retrovirus. Journal of Virology 73, 2365-2375 (1999).

Kotewicz, M. L., Sampson. C. M., D'Alessio, J. M. & Gerard. G. F. Isolation of cloned Moloney murine leukemia virus reverse transcriptase lacking ribonuclease H activity. Nucleic Acids Res 16, 265-277 (1988).

Arezi, B. & Hogrefe, H. Novel mutations in Moloney Murine Leukemia Virus reverse transcriptase increase thermostability through tighter binding to template-primer. Nucleic Acids Res 37, 473-481 (2009).

Blain, S. W. & Goff, S. P. Nuclease activities of Moloney murine leukemia virus reverse transcriptase. Mutants with altered substrate specificities. J. Biol. Chem. 268, 23585-23592 (1993).

Xiong. Y. & Eickbush, T. H. Origin and evolution of retroelements based upon their reverse transcriptase sequences. EMBO J 9, 3353-3362 (1990). Herschhom, A. & Hizi. A. Retroviral reverse transcriptases. Cell. Mol. Life Sci. 67, 2717-2747 (2010).

Taube, R., Loya, S., Avidan, O., Perach, M. & Hizi, A. Reverse transcriptase of mouse mammary tumour virus: expression in bacteria, purification and biochemical characterization. Biochem. J. 329 (Pt 3), 579-587 (1998).

Liu, M. et al. Reverse Transcriptase-Mediated Tropism Switching in Bordetella Bacteriophage. Science 295, 2091-2094 (2002).

Luan. D. D., Korman, M. H., Jakubczak, J. L. & Eickbush. T. H. Reverse transcription of R2Bm RNA is primed by a nick at the chromosomal target site: a mechanism for non-LTR retrotransposition. Cell 72, 595-605 (1993).

Nottingham, R. M. et al. RNA-seq of human reference RNA samples using a thermostable group II intron reverse transcriptase. RNA 22, 597-613 (2016).

Telesnitsky, A. & Goff, S. P. RNase H domain mutations affect the interaction between Moloney murine leukemia virus reverse transcriptase and its primer-template. Proc. Natl. Acad. Sci. U.S.A. 90, 1276-1280 (1993).

Halvas, E. K., Svarovskaia, E. S. & Pathak, V. K. Role of Murine Leukemia Virus Reverse Transcriptase Deoxyribonucleoside Triphosphate-Binding Site in Retroviral Replication and In Vivo Fidelity. Journal of Virology 74, 10349-10358 (2000).

Nowak, E. et al. Structural analysis of monomeric retroviral reverse transcriptase in complex with an RNA/DNA hybrid. Nucleic Acids Res 41, 3874-3887 (2013).

Stamos, J. L., Lentzsch, A. M. & Lambowitz, A. M. Structure of a Thermostable Group II Intron Reverse Transcriptase with Template-Primer and Its Functional and Evolutionary Implications. Molecular Cell 68, 926-939.e4 (2017).

Das, D. & Georgiadis. M. M. The Crystal Structure of the Monomeric Reverse Transcriptase from Moloney Murine Leukemia Virus. Structure 12, 819-829 (2004).

Avidan, O., Meer, M. E., Oz, 1. & Hizi, A. The processivity and fidelity of DNA synthesis exhibited by the reverse transcriptase of bovine leukemia virus. European Journal of Biochemistry 269, 859-867 (2002).

Gerard, G. F. et al. The role of template-primer in protection of reverse transcriptase from thermal inactivation. Nucleic Acids Res 30, 3118-3129 (2002).

Monot, C. et al. The Specificity and Flexibility of L1 Reverse Transcription Priming at Imperfect T-Tracts. PLOS Genetics 9, e1003499 (2013).

Mohr, S. et al. Thermostable group II intron reverse transcriptase fusion proteins and their use in cDNA synthesis and next-generation RNA sequencing. RNA 19, 958-970 (2013).

Any of the references noted above which relate to reverse transcriptases are hereby incorporated by reference in their entireties, if not already stated so.

### Prime Editors: Fusion Proteins

The napDNAbps and the polymerases (e.g., reverse transcriptases) may be provided in the form of a fusion protein. That is, the present disclosure contemplates using prime editors comprising fusion proteins, wherein the fusion proteins comprise a napDNAbp domain and a polymerase (e.g., reverse transcriptase) domain.

The prime editor systems utilized in the methods and compositions described herein contemplate fusion proteins comprising a napDNAbp and a polymerase (e.g., DNA-dependent DNA polymerase or RNA-dependent DNA polymerase, such as, reverse transcriptase), and optionally joined by a linker. The application contemplates any suitable napDNAbp and polymerase (e.g., DNA-dependent DNA polymerase or RNA-dependent DNA polymerase, such as, reverse transcriptase) to be combined in a single fusion protein. Examples of napDNAbps and polymerases (e.g., DNA-dependent DNA polymerase or RNA-dependent DNA polymerase, such as, reverse transcriptase) are each defined herein. Since polymerases are well-known in the art, and the amino acid sequences are readily available, this disclosure is not meant in any way to be limited to those specific polymerases identified herein.

In various embodiments, the fusion proteins may comprise any suitable structural configuration. For example, the fusion protein may comprise from the N-terminus to the C-terminus direction, a napDNAbp fused to a polymerase (e.g., DNA-dependent DNA polymerase or RNA-dependent DNA polymerase, such as, reverse transcriptase). In other embodiments, the fusion protein may comprise from the N-terminus to the C-terminus direction, a polymerase (e.g., a reverse transcriptase) fused to a napDNAbp. The fused domain may optionally be joined by a linker, e.g., an amino acid sequence. In other embodiments, the fusion proteins may comprise the structure NH₂-[napDNAbp]-[polymerase]-COOH; or NH₂-[polymerase]-[napDNAbp]-C00H, wherein each instance of “]-[” indicates the presence of an optional linker sequence. In embodiments wherein the polymerase is a reverse transcriptase, the fusion proteins may comprise the structure NH-2-[napDNAbp]-[RT]-COOH; or NH₂-[RT]-[napDNAbp]-COOH, wherein each instance of “]-[” indicates the presence of an optional linker sequence.

In various embodiments, the prime editor fusion protein may have the following amino acid sequence (referred to herein as “PE1”), which includes a Cas9 variant comprising an H840A mutation (i.e., a Cas9 nickase) and an M-MLV RT wild type, as well as an N-terminal NLS sequence (19 amino acids) and an amino acid linker (32 amino acids) that joins the C-terminus of the Cas9 nickase domain to the N-terminus of the RT domain. The PE1 fusion protein has the following structure: [NLS]-[Cas9(H840A)]-[linker]-[MMLV_RT(wt)]. The amino acid sequence of PE1 and its individual components are as follows:

| Description | Sequence |
|---|---|
| PEI FUSION | MKRTADGSEFESPKKKRKVDKKYSIGLDIG |
| PROTEIN | TNSVGWAVITDEYKVPSKKFKVLGNTDRHS |
| CAS9(H840A)- | IKKNLIGALLFDSGETAEATRLKRTARRRY |
| MMLV_RT(WT) | TRRKNRICYLQEIFSNEMAKVDDSFFHRLE |
| | ESELVEEDKKHERHPIFGNIVDEVAYHEKY |
| | PTIYHLRKKLVDSTDKADLRLIYLALAHMI |
| | KFRGHFLIEGDLNPDNSDVDKLFIQLVQTY |
| | NQLFEENPINASGVDAKAILSARLSKSRRL |
| | ENLIAQLPGEKKNGLFGNLIALSLGLTPNF |
| | KSNEDLAEDAKLQLSKDTYDDDLDNLLAQI |
| | GDQYADLFLAAKNLSDAILLSDILRVNTEI |
| | TKAPLSASMIKRYDEHHQDLTLLKALVRQQ |
| | LPEKYKEIFFDQSKNGYAGYIDGGASQEEF |
| | YKFIKPILEKMDGTEELLVKLNREDLLRKQ |
| | RTEDNGSIPHQIHLGELHAILRRQEDFYPF |
| | LKDNREKIEKILTFRIPYYVGPLARGNSRF |
| | AWMTRKSEETITPWNFEEVVDKGASAQSFI |
| | ERMTNFDKNLPNEKVLPKHSLLYEYFTVYN |
| | ELTKVKYVTEGMRKPAFLSGEQKKAIVDLL |
| | FKTNRKVTVKQLKEDYFKKIECFDSVEISG |
| | VEDRFNASLGTYHDLLKIIKDKDFLDNEEN |
| | EDILEDIVLTLTLFEDREMIEERLKTYAHL |
| | EDDKVMKQLKRRRYTGWGRLSRKLINGIRD |
| | KQSGKTILDELKSDGFANRNFMQLIHDDSL |
| | TFKEDIQKAQVSGQGDSLHEHIANLAGSPA |
| | IKKGILQTVKVVDELVKVMGRHKPENIVIE |
| | MARENQTTQKGQKNSRERMKRIEEGIKELG |
| | SQILKEHPVENTQLQNEKLYLYYLQNGRDM |
| | YVDQELDINRLSDYDVDAIVPQSFLKDDSI |
| | DNKVLTRSDKNRGKSDNVPSEEVVKKMKNY |
| | WRQLLNAKLITQRKFDNLTKAERGGLSELD |
| | KAGFIKRQLVETRQITKHVAQILDSRMNTK |
| | YDENDKLIREVKVITLKSKLVSDERKDFQF |
| | YKVREINNYHHAHDAYLNAVVGTALIKKYP |
| | KLESEFVYGDYKVYDVRKMIAKSEQEIGKA |
| | TAKYFFYSNIMNFFKTEITLANGEIRKRPL |
| | IETNGETGEIVWDKGRDFATVRKVLSMPQV |
| | NIVKKTEVQTGGFSKESILPKRNSDKLIAR |
| | KKDWDPKKYGGFDSPTVAYSVLVVAKVEKG |
| | KSKKLKSVKELLGITIMERSSFEKNPIDFL |
| | EAKGYKEVKKDLIIKLPKYSLFELENGRKR |
| | MLASAGELQKGNELALPSKYVNFLYLASHY |
| | EKLKGSPEDNEQKQLFVEQHKHYLDEIIEQ |
| | ISEFSKRVILADANLDKVLSAYNKHRDKPI |
| | REQAENIIHLFTLTNLGAPAAFKYFDTTID |
| | RKRYTSTKEVLDATLIHQSITGLYETRIDL |
| | SQLGGDSGGSSGGSSGSETPGTSESATPES |
| | SGGSSGGSSTLNIEDEYRLHETSKEPDVSL |
| | GSTWLSDFPQAWAETGGMGLAVRQAPLIIP |
| | LKATSTPVSIKQYPMSQEARLGIKPHIQRL |
| | LDQGILVPCQSPWNTPLLPVKKPGTNDYRP |
| | VQDLREVNKRVEDIHPTVPNPYNLLSGLPP |
| | SHQWYTVLDLKDAFFCLRLHPTSQPLFAFE |
| | WRDPEMGISGQLTWTRLPQGFKNSPTLFDE |
| | ALHRDLADFRIQHPDLILLQYVDDLLLAAT |
| | SELDCQQGTRALLQTLGNLGYRASAKKAQI |
| | CQKQVKYLGYLLKEGQRWLTEARKETVMGQ |
| | PTPKTPRQLREFLGTAGFCRLWIPGFAEMA |
| | APLYPLTKTGTLFNWGPDQQKAYQEIKQAL |
| | LTAPALGLPDLTKPFELFVDEKQGYAKGVL |
| | TQKLGPWRRPVAYLSKKLDPVAAGWPPCLR |
| | MVAAIAVLTKDAGKLIMGQPLVILAPHAVE |
| | ALVKQPPDRWLSNARMTHYQALLLDTDRVQ |
| | FGPVVALNPATLLPLPEEGLQHNCLDILAE |
| | AHGTRPDLTDQPLPDADHTWYTDGSSLLQE |
| | GQRKAGAAVTTETEVIWAKALPAGTSAQRA |
| | ELIALTQALKMAEGKKLNVYTDSRYAFATA |
| | HIHGEIYRRRGLLTSEGKEIKNKDEILALL |
| | KALFLPKRLSIIHCPGHQKGHSAEARGNRM |
| | ADQAARKAAITETPDTSTLLIENSSPSGGS |
| | KRTADGSEFEPKKKRKV |
| | (SEQ ID NO: 100) |
| | KEY: |
| | NUCLEAR LOCALIZATION SEQUENCE (NLS) TOP: |
| | (SEQ ID NO: 101), |
| | BOTTOM: (SEQ ID NO: 103) |
| | CAS9(H840A) (SEQ ID NO: 37) |
| | 33-AMINO ACID LINKER (SEQ ID NO: 102) |
| | M-MLV reverse transcriptase (SEQ ID NO: 81) |
| PE1-N-TERMINAL | MKRTADGSEFESPKKKRKV (SEQ ID NO: 101) |
| NLS | |
| PEI-CAS9 (H840A) | DKKYSIGLDIGTNSVGWAVITDEYKVPSKK |
| (MET MINUS) | FKVLGNTDRHSIKKNLIGALLFDSGETAEA |
| | TRLKRTARRRYTRRKNRICYLQEIFSNEMA |
| | KVDDSFFHRLEESFLVEEDKKHERHPIFGN |
| | IVDEVAYHEKYPTIYHLRKKLVDSTDKADL |
| | RLIYLALAHMIKFRGHFLIEGDLNPDNSDV |
| | DKLFIQLVQTYNQLFEENPINASGVDAKAI |
| | LSARLSKSRRLENLIAQLPGEKKNGLFGNL |
| | IALSLGLTPNFKSNFDLAEDAKLQLSKDTY |
| | DDDLDNLLAQIGDQYADLFLAAKNLSDAIL |
| | LSDILRVNTEITKAPLSASMIKRYDEHHQD |
| | LTLLKALVRQQLPEKYKEIFFDQSKNGYAG |
| | YIDGGASQEEFYKFIKPILEKMDQTEELLV |
| | KLNREDLLRKQRTFDNGSIPHQIHLGELHA |
| | ILRRQEDFYPFLKDNREKIEKILTFRIPYY |
| | VGPLARGNSRFAWMTRKSEETITPWNFEEV |
| | VDKGASAQSFIERMTNFDKNLPNEKVLPKH |
| | SLLYEYFTVYNELTKVKYVTEGMRKPAFLS |
| | GEQKKAIVDLLFKTNRKVTVKQLKEDYFKK |
| | IECFDSVEISGVEDRENASLGTYHDLLKII |
| | KDKDELDNEENEDILEDIVLTLTLFEDREM |
| | IEERLKTYAHLEDDKVMKQLKRRRYTGWGR |
| | LSRKLINGIRDKQSGKTILDFLKSDGFANR |
| | NFMQLIHDDSLTFKEDIQKAQVSGQGDSLH |
| | EHIANLAGSPAIKKGILQTVKVVDELVKVM |
| | GRHKPENIVIEMARENQTTQKGQKNSRERM |
| | KRIEEGIKELGSQILKEHPVENTQLQNEKL |
| | YLYYLQNGRDMYVDQELDINRLSDYDVDAI |
| | VPQSFLKDDSIDNKVLTRSDKNRGKSDNVP |
| | SEEVVKKMKNYWRQLLNAKLITQRKEDNLT |
| | KAERGGLSELDKAGFIKRQLVETRQITKHV |
| | AQILDSRMNTKYDENDKLIREVKVITLKSK |
| | LVSDFRKDFQFYKVREINNYHHAHDAYLNA |
| | VVGTALIKKYPKLESEFVYGDYKVYDVRKM |
| | IAKSEQEIGKATAKYFFYSNIMNFFKTEIT |
| | LANGEIRKRPLIETNGETGEIVWDKGRDFA |
| | TVRKVLSMPQVNIVKKTEVQTGGFSKESIL |
| | PKRNSDKLIARKKDWDPKKYGGFDSPTVAY |
| | SVLVVAKVEKGKSKKLKSVKELLGITIMER |
| | SSFEKNPIDFLEAKGYKEVKKDLUIKLPKY |
| | SLFELENGRKRMLASAGELQKGNELALPSK |
| | YVNFLYLASHYEKLKGSPEDNEQKQLFVEQ |
| | HKHYLDEIIEQISEFSKRVILADANLDKVL |
| | SAYNKHRDKPIREQAENIIHLFTLTNLGAP |
| | AAFKYFDTTIDRKRYTSTKEVLDATLIHQS |
| | ITGLYETRIDLSQLGGD |
| | (SEQ ID NO: 37) |
| PEI-LINKER | SGGSSGGSSGSETPGTSESATPESSGGSSG |
| BETWEEN CAS9 | GSS (SEQ ID NO: 102) |
| DOMAIN AND RT | |
| DOMAIN (33 AMINO | |
| ACIDS) | |
| PE1-M-MLV RT | TLNIEDEYRLHETSKEPDVSLGSTWLSDFP |
| | QAWAETGGMGLAVRQAPLIIPLKATSTPVS |
| | IKQYPMSQEARLGIKPHIQRLLDQGILVPC |
| | QSPWNTPLLPVKKPGTNDYRPVQDLREVNK |
| | RVEDIHPTVPNPYNLLSGLPPSHQWYTVLD |
| | LKDAFFCLRLHPTSQPLFAFEWRDPEMGIS |
| | GQLTWTRLPQGFKNSPTLFDEALHRDLADF |
| | RIQHPDLILLQYVDDLLLAATSELDCQQGT |
| | RALLQTLGNLGYRASAKKAQICQKQVKYLG |
| | YLLKEGQRWLTEARKETVMGQPTPKTPRQL |
| | REFLGTAGFCRLWIPGFAEMAAPLYPLTKT |
| | GTLFNWGPDQQKAYQEIKQALLTAPALGLP |
| | DLTKPFELFVDEKQGYAKGVLTQKLGPWRR |
| | PVAYLSKKLDPVAAGWPPCLRMVAAIAVLT |
| | KDAGKLTMGQPLVILAPHAVEALVKQPPDR |
| | WLSNARMTHYQALLLDTDRVQFGPVVALNP |
| | ATLLPLPEEGLQHNCLDILAEAHGTRPDLT |
| | DQPLPDADHTWYTDGSSLLQEGQRKAGAAV |
| | TTETEVIWAKALPAGTSAQRAELIALTQAL |
| | KMAEGKKLNVYTDSRYAFATAHIHGEIYRR |
| | RGLLTSEGKEIKNKDEILALLKALFLPKRL |
| | SIIHCPGHQKGHSAEARGNRMADQAARKAA |
| | ITETPDTSTLLIENSSP |
| | (SEQ ID NO: 81) |
| PEI-C-TERMINAL NLS | SGGSKRTADGSEFEPKKKRKV |
| | (SEQ ID NO: 103) |

In another embodiment, the prime editor fusion protein may have the following amino acid sequence (referred to herein as “PE2”), which includes a Cas9 variant comprising an H840A mutation (i.e., a Cas9 nickase) and an M-MLV RT comprising mutations D200N, T330P, L603W, T306K, and W313F, as well as an N-terminal NLS sequence (19 amino acids) and an amino acid linker (33 amino acids) that joins the C-terminus of the Cas9 nickase domain to the N-terminus of the RT domain. The PE2 fusion protein has the following structure: [NLS]-[Cas9(H840A)]-[linker]-[MMLV_RT(D200N)(T330P)(L603W)(T306K)(W3I3F)]. The amino acid sequence of PE2 is as follows:

| | |
|---|---|
| PE2 FUSION PROTEIN | MKRTADGSEFESPKKKRKVDKKYSIGLDIGTNSVGWAVITDEYKV |
| CAS9(H840A)-MMLV_RT | PSKKFKVLGNTDRHSIKKNLIGALLFDSGETAEATRLKRTARRRY |
| D200N | TRRKNRICYLQEIFSNEMAKVDDSFFHRLEESFLVEEDKKHERHP |
| T330P | IFGNIVDEVAYHEKYPTIYHLRKKLVDSTDKADLRLIYLALAHMI |
| L603W | KERGHFLIEGDLNPDNSDVDKLFIQLVQTYNQLFEENPINASGVD |
| T306K | AKAILSARLSKSRRLENLIAQLPGEKKNGLFGNLIALSLGLTPNF |
| W313F | KSNFDLAEDAKLQLSKDTYDDDLDNLLAQIGDQYADLFLAAKNLS |
| | DAILLSDILRVNTEITKAPLSASMIKRYDEHHQDLTLLKALVRQQ |
| | LPEKYKEIFFDQSKNGYAGYIDGGASQEEFYKFIKPILEKMDGTE |
| | ELLVKLNREDLLRKQRTFDNGSIPHQIHLGELHAILRRQEDFYPF |
| | LKDNREKIEKILTFRIPYYVGPLARGNSRFAWMTRKSEETITPWN |
| | FEEVVDKGASAQSFIERMTNEDKNLPNEKVLPKHSLLYEYFTVYN |
| | ELTKVKYVTEGMRKPAFLSGEQKKAIVDLLFKTNRKVTVKQLKED |
| | YFKKIECFDSVEISGVEDRFNASLGTYRDLLKIIKDKDELDNEEN |
| | EDILEDIVLTLTLFEDREMIEERLKTYAHLFDDKVMKQLKRRRYT |
| | GWGRLSRKLINGIRDKQSGKTILDFLKSDGFANRNFMQLIHDDSL |
| | TFKEDIQKAQVSGQGDSLHEHIANLAGSPAIKKGILQTVKVVDEL |
| | VKVMGRHKPENIVIEMARENQTTQKGQKNSRERMKRIEEGIKELG |
| | SQILKEHPVENTQLQNEKLYLYYLQNGRDMYVDQELDINRLSDYD |
| | VDAIVPQSFLKDDSIDNKVLTRSDKNRGKSDNVPSEEVVKKMKNY |
| | WRQLLNAKLITQRKFDNLTKAERGGLSELDKAGFIKRQLVETRQI |
| | TKHVAQILDSRMNTKYDENDKLIREVKVITLKSKLVSDERKDFQF |
| | YKVREINNYHHAHDAYLNAVVGTALIKKYPKLESEFVYGDYKVYD |
| | VRKMIAKSEQEIGKATAKYFFYSNIMNFFKTEITLANGEIRKRPL |
| | IETNGETGEIVWDKGRDFATVRKVLSMPQVNIVKKTEVQTGGFSK |
| | ESILPKRNSDKLIARKKDWDPKKYGGFDSPTVAYSVLVVAKVEKG |
| | KSKKLKSVKELLGITIMERSSFEKNPIDFLEAKGYKEVKKDLIIK |
| | LPKYSLFELENGRKRMLASAGELQKGNELALPSKYVNFLYLASHY |
| | EKLKGSPEDNEQKQLFVEQHKHYLDEIIEQISEFSKRVILADANL |
| | DKVLSAYNKHRDKPIREQAENIIHLFTLINLGAPAAFKYEDTTID |
| | RKRYTSTKEVLDATLIHQSITGLVETRIDLSQLGGDSGGSSGGSS |
| | GSETPGTSESATPESSGGSSGGSSTLNIEDEYRLHETSKEPDVSL |
| | GSTWLSDFPQAWAETGGMGLAVRQAPLIIPLKATSTPVSIKQYPM |
| | SQEARLGIKPHIQRLLDQGILVPCQSPWNTPLLPVKKPGTNDYRP |
| | VQDLREVNKRVEDIHPTVPNPYNLLSGLPPSHQWYTVLDLKDAFF |
| | CLRLHPTSQPLFAFEWRDPEMGISGQLTWTRLPQGFKNSPTLFNE |
| | ALHRDLADFRIQHPDLILLQYVDDLLLAATSELDCQQGTRALLQT |
| | LGNLGYRASAKKAQICQKQVKYLGYLLKEGQRWLTEARKETVMGQ |
| | PTPKTPRQLREFLGKAGFCRLFIPGFAEMAAPLYPLTKPGTLFNW |
| | GPDQQKAYQEIKQALLTAPALGLPDLTKPFELFVDEKQGYAKGVL |
| | TQKLGPWRRPVAYLSKKLDPVAAGWPPCLRMVAAIAVLTKDAGKL |
| | TMGQPLVILAPHAVEALVKQPPDRWLSNARMTHYQALLLDTDRVQ |
| | FGPVVALNPATLLPLPEEGLQHNCLDILAEAHGTRPDLTDQPLPD |
| | ADHTWYTDGSSLLQEGQRKAGAAVTTETEVIWAKALPAGTSAQRA |
| | ELIALTQALKMAEGKKLNVYTDSRYAFATAHIHGEIYRRRGWLTS |
| | EGKEIKNKDEILALLKALFLPKRLSIIHCPGHQKGHSAEARGNRM |
| | ADQAARKAAITETPDTSTLLIENSSPSGGSKRTADGSEFEPKKKR |
| | KV |
| | (SEQ ID NO: 107) |
| | KEY: |
| | NUCLEAR LOCALIZATION SEQUENCE (NLS) TOP: (SEQ |
| | ID NO: 101), |
| | BOTTOM: (SEQ ID NO: 103) |
| | CAS9(H840A) (SEQ ID NO: 37) |
| | 33-AMINO ACID LINKER (SEQ ID NO: 102) |
| | M-MLV reverse transcriptase (SEQ ID NO: 98) |
| PE2-N-TERMINAL NLS | DKKYSIGLDIGTNSVGWAVITDEYKVPSKKFKVLGNTDRHSIKKN |
| | LIGALLFDMKRTADGSEFESPKKKRKV |
| | (SEQ ID NO: 101) |
| PE2-CAS9 (H840A) | SGETAEATRLKRTARRRYTRRKNRICYLQEIFSNEMAKVDDSFFH |
| (MET MINUS) | RLEESFLVEEDKKHERHPIFGNIVDEVAYHEKYPTIYHLRKKLVD |
| | STDKADLRLIYLALAHMIKFRGHFLIEGDLNPDNSDVDKLFIQLV |
| | QTYNQLFEENPINASGVDAKAILSARLSKSRRLENLIAQLPGEKK |
| | NGLFGNLIALSLGLTPNFKSNFDLAEDAKLQLSKDTYDDDLDNLL |
| | AQIGDQYADLFLAAKNLSDAILLSDILRVNTEITKAPLSASMIKR |
| | YDEHHQDLTLLKALVRQQLPEKYKEIFFDQSKNGYAGYIDGGASQ |
| | EEFYKFIKPILEKMDGTEELLVKLNREDLLRKQRTFDNGSIPHQI |
| | HLGELHAILRRQEDFYPFLKDNREKIEKILTFRIPYYVGPLARGN |
| | SRFAWMTRKSEETITPWNFEEVVDKGASAQSFIERMTNFDKNLPN |
| | EKVLPKHSLLYEYFTVYNELTKVKYVTEGMRKPAFLSGEQKKAIV |
| | DLLFKTNRKVTVKQLKEDYFKKIECFDSVEISGVEDRFNASLGTY |
| | HDLLKIIKDKDFLDNEENEDILEDIVLTLTLFEDREMIEERLKTY |
| | AHLFDDKVMKQLKRRRYTGWGRLSRKLINGIRDKQSGKTILDFLK |
| | SDGFANRNFMQLIHDDSLTFKEDIQKAQVSGQGDSLHEHIANLAG |
| | SPAIKKGILQTVKVVDELVKVMGRHKPENIVIEMARENQTTQKGQ |
| | KNSRERMKRIEEGIKELGSQILKEHPVENTQLQNEKLYLYYLQNG |
| | RDMYVDQELDINRLSDYDVDAIVPQSFLKDDSIDNKVLTRSDKNR |
| | GKSDNVPSEEVVKKMKNYWRQLLNAKLITQRKFDNLTKAERGGLS |
| | ELDKAGFIKRQLVETRQITKHVAQILDSRMNTKYDENDKLIREVK |
| | VITLKSKLVSDFRKDFQFYKVREINNYHHAHDAYLNAVVGTALIK |
| | KYPKLESEFVYGDYKVYDVRKMIAKSEQEIGKATAKYFFYSNIMN |
| | FFKTEITLANGEIRKRPLIETNGETGEIVWDKGRDFATVRKVLSM |
| | PQVNIVKKTEVQTGGFSKESILPKRNSDKLIARKKDWDPKKYGGF |
| | DSPTVAYSVLVVAKVEKGKSKKLKSVKELLGITIMERSSFEKNPI |
| | DFLEAKGYKEVKKDLIIKLPKYSLFELENGRKRMLASAGELQKGN |
| | ELALPSKYVNFLYLASHYEKLKGSPEDNEQKQLFVEQHKHYLDEI |
| | IEQISEFSKRVILADANLDKVLSAYNKHRDKPIREQAENIIHLFT |
| | LTNLGAPAAFKYFDTTIDRKRYTSTKEVLDATLIHQSITGLYETR |
| | IDLSQLGGD |
| | (SEQ ID NO: 37) |
| PE2-LINKER BETWEEN | SGGSSGGSSGSETPGTSESATPESSGGSSGGSS |
| CAS9 DOMAIN AND RT | (SEQ ID NO: 102) |
| DOMAIN (33 AMINO | |
| ACIDS) | |
| PE2-MMLV_RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPC |
| T330P | QSPWNTPLLPVKKPGTNDYRPVQDLREVNKRVEDIHPTVPNPYNL |
| L603W | LSGLPPSHQWYTVLDLKDAFFCLRLHPTSQPLFAFEWRDPEMGIS |
| T306K | GQLTWTRLPQGFKNSPTLFNEALHRDLADFRIQHPDLILLQYVDD |
| W313F | LLLAATSELDCQQGTRALLQTLQNLGYRASAKKAQICQKQVKYLQ |
| | YLLKEGQRWLTEARKETVMGQPTPKTPRQLREFLGKAGFCRLFIP |
| | GFAEMAAPLYPLTKPGTLFNWGPDQQKAYQEIKQALLTAPALGLP |
| | DLTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHNC |
| | LDILAEAHGTRPDLTDQPLPDADHTWYTDGSSLLQEGQRKAGAAV |
| | TTETEVIWAKALPAGTSAQRAELIALTQALKMAEGKKLNVYTDSR |
| | YAFATAHIHGETYRRRGWLTSEGKEIKNKDEILALLKALFLPKRL |
| | SIIHCPGHQKGHSAEARGNRMADQAARKAAITETPDTSTLLIENS |
| | SP |
| | (SEQ ID NO: 98) |
| PE2-C-TERMINAL NLS | SGGSKRTADGSEFEPKKKRKV (SEQ ID NO: 103) |

In still other embodiments, the prime editor fusion protein may have the following amino acid sequences:

| | |
|---|---|
| PE FUSION PROTEIN | MKRTADGSEFESPKKKRKVTLNIEDEYRLHETSKEPDVSLGSTWL |
| MMLV_RT(WT)- | SDFPQAWAETGGMGLAVRQAPLIIPLKATSTPVSIKQYPMSQEAR |
| 32AA-CAS9(H840A) | LGIKPHIQRLLDQGILVPCQSPWNTPLLPVKKPGTNDYRPVQDLR |
| | EVNKRVEDIHPTVPNPYNLLSGLPPSHQWYTVLDLKDAFFCLRLH |
| | PTSQPLFAFEWRDPEMGISGQLTWTRLPQGFKNSPTLFDEALHRD |
| | LADFRIQHPDLILLQYVDDLLLAATSELDCQQGTRALLQTLGNLG |
| | YRASAKKAQICQKQVKYLGYLLKEGQRWLTEARKETVMGQPTPKT |
| | PRQLREFLGTAGFCRLWIPGFAEMAAPLYPLTKTGTLFNWGPDQQ |
| | KAYQEIKQALLTAPALGLPDLTKPFELFVDEKQGYAKGVLTQKLG |
| | PWRRPVAYLSKKLDPVAAGWPPCLRMVAAIAVLTKDAGKLTMGQP |
| | LVILAPHAVEALVKQPPDRWLSNARMTHYQALLLDTDRVQFGPVV |
| | ALNPATLLPLPEEGLQHNCLDILAEAHGTRPDLTDQPLPDADHTW |
| | YTDGSSLLQEGQRKAGAAVTTETEVIWAKALPAGTSAQRAELIAL |
| | TQALKMAEGKKLNVYTDSRYAFATAHIHGEIYRRRGLLTSEGKEI |
| | KNKDEILALLKALFLPKRLSIIHCPGHQKGHSAEARGNRMADQAA |
| | RKAAITETPDTSTLLIENSSPSGGSSGGSSGSETPGTSESATPES |
| | SGGSSGGSSDKKYSIGLDIGTNSVGWAVITDEYKVPSKKFKVLGN |
| | TDRHSIKKNLIGALLEDSGETAEATRLKRTARRRYTRRKNRICYL |
| | QEIFSNEMAKVDDSFFHRLEESFLVEEDKKHERHPIFGNIVDEVA |
| | YHEKYPTIYHLRKKLVDSTDKADLRLIYLALAHMIKFRGHFLIEG |
| | DLNPDNSDVDKLFIQLVQTYNQLFEENPINASGVDAKAILSARLS |
| | KSRRLENLIAQLPGEKKNGLFGNLIALSLGLTPNFKSNFDLAEDA |
| | KLQLSKDTYDDDLDNLLAQIGDQYADLFLAAKNLSDAILLSDILR |
| | VNTEITKAPLSASMIKRYDEHHQDLTLLKALVRQQLPEKYKEIFF |
| | DQSKNGYAGYIDGGASQEEFYKFIKPILEKMDGTEELLVKLNRED |
| | LLRKQRTFDNGSIPHQIHLGELHAILRRQEDFYPFLKDNREKIEK |
| | ILTFRIPYYVGPLARGNSRFAWMTRKSEETITPWNFEEVVDKGAS |
| | AQSFIERMTNEDKNLPNEKVLPKHISLLYEYFTVYNELTKVKYVT |
| | EGMRKPAFLSGEQKKAIVDLLFKTNRKVTVKQLKEDYFKKIECFD |
| | SVEISGVEDRFNASLGTYHDLLKIIKDKDFLDNEENEDILEDIVL |
| | TLTLFEDREMIEERLKTYAHLEDDKVMKQLKRRRYTGWGRLSRKL |
| | INGIRDKQSGKTILDFLKSDGFANRNEMQLIHDDSLTFKEDIQKA |
| | QVSGQGDSLHEHIANLAGSPAIKKGILQTVKVVDELVKVMGRHKP |
| | ENIVIEMARENQTTQKGQKNSRERMKRIEEGIKELGSQILKEHPV |
| | ENTQLQNEKLYLYYLQNGRDMYVDQELDINRLSDYDVDAIVPQSF |
| | LKDDSIDNKVLTRSDKNRGKSDNVPSEEVVKKMKNYWRQLLNAKL |
| | ITQRKFDNLTKAERGGLSELDKAGFIKRQLVETRQITKHVAQILD |
| | SRMNTKYDENDKLIREVKVITLKSKLVSDFRKDFQFYKVREINNY |
| | HHAHDAYLNAVVGTALIKKYPKLESEFVYGDYKVYDVRKMIAKSE |
| | QEIGKATAKYFFYSNIMNFFKTEITLANGEIRKRPLIETNGETGE |
| | IVWDKGRDFATVRKVLSMPQVNIVKKTEVQTGGFSKESILPKRNS |
| | DKLIARKKDWDPKKYGGFDSPTVAYSVLVVAKVEKGKSKKLKSVK |
| | ELLGITIMERSSFEKNPIDFLEAKGYKEVKKDLIIKLPKYSLFEL |
| | ENGRKRMLASAGELQKGNELALPSKYVNFLYLASHYEKLKGSPED |
| | NEQKQLFVEQHKHYLDEHIEQISEFSKRVILADANLDKVLSAYNK |
| | HRDKPIREQAENIIHLFTLINLGAPAAFKYFDTTIDRKRYTSTKE |
| | VLDATLIHQSITGLYETRIDLSQLGGDSGGSKRTADGSEFEPKKK |
| | RKV |
| | (SEQ ID NO: 108) |
| | KEY: |
| | NUCLEAR LOCALIZATION SEQUENCE (NLS) TOP:(SEQ |
| | ID NO: 101), |
| | BOTTOM: (SEQ ID NO: 103) |
| | CAS9(H840A) (SEQ ID NO: 37) |
| | 33-AMINO ACID LINKER (SEQ ID NO: 102) |
| | M-MLV reverse transcriptase (SEQ ID NO: 81) |
| PE FUSION PROTEIN | MKRTADGSEFESPKKKRKVTLNIEDEYRLHETSKEPDVSLGSTWL |
| MMLV_RT(WT)- | SDFPQAWAETGGMGLAVRQAPLIIPLKATSTPVSIKQYPMSQEAR |
| 60AA-CAS9(H840A) | LGIKPHIQRLLDQGILVPCQSPWNTPLLPVKKPGTNDYRPVQDLR |
| | EVNKRVEDIHPTVPNPYNLLSGLPPSHQWYTVLDLKDAFFCLRLH |
| | PTSQPLFAFEWRDPEMGISGQLTWTRLPQGFKNSPTLFDEALHRD |
| | LADFRIQHPDLILLQYVDDLLLAATSELDCQQGTRALLQTLGNLG |
| | YRASAKKAQICQKQVKYLGYLLKEGQRWLTEARKETVMGQPTPKT |
| | PRQLREFLGTAGFCRLWIPGFAEMAAPLYPLTKTGTLFNWGPDQQ |
| | KAYQEIKQALLTAPALGLPDLTKPFELFVDEKQGYAKGVLTQKLG |
| | PWRRPVAYLSKKLDPVAAGWPPCLRMVAAIAVLTKDAGKLTMGQP |
| | LVILAPHAVEALVKQPPDRWLSNARMTHYQALLLDTDRVQFGPVV |
| | ALNPATLLPLPEEGLQHNCLDILAEAHGTRPDLTDQPLPDADHTW |
| | YTDGSSLLQEGQRKAGAAVTTETEVIWAKALPAGTSAQRAELIAL |
| | TQALKMAEGKKLNVYTDSRYAFATAHIHGEIYRRRGLLTSEGKEI |
| | KNKDEILALLKALFLPKRLSIIHCPGHQKGHSAEARGNRMADQAA |
| | RKAAITETPDTSTLLIENSSPSGGSSGGSSGSETPGTSESATPES |
| | AGSYPYDVPDYAGSAAPAAKKKKLDGSGSGGSSGGSDKKYSIGLD |
| | IGTNSVGWAVITDEYKVPSKKFKVLGNTDRHSIKKNLIGALLFDS |
| | GETAEATRLKRTARRRYTRRKNRICYLQEIFSNEMAKVDDSFFHR |
| | LEESFLVEEDKKHERHPIFGNIVDEVAYHEKYPTIYHLRKKLVDS |
| | TDKADLRLIYLALAHMIKFRGHFLIEGDLNPDNSDVDKLFIQLVQ |
| | TYNQLFEENPINASGVDAKAILSARLSKSRRLENLIAQLPGEKKN |
| | GLFGNLIALSLGLTPNFKSNFDLAEDAKLQLSKDTYDDDLDNLLA |
| | QIGDQYADLFLAAKNLSDAILLSDILRVNTEITKAPLSASMIKRY |
| | DEHHQDLTLLKALVRQQLPEKYKEIFFDQSKNGYAGYIDGGASQE |
| | EFYKFIKPILEKMDGTEELLVKLNREDLLRKQRTFDNGSIPHQIH |
| | LGELHAILRRQEDFYPFLKDNREKIEKILTFRIPYYVGPLARGNS |
| | RFAWMTRKSEETITPWNFEEVVDKGASAQSFIERMTNFDKNLPNE |
| | KVLPKHSLLYEYFTVYNELTKVKYVTEGMRKPAFLSGEQKKAIVD |
| | LLFKTNRKVTVKQLKEDYFKKIECFDSVEISGVEDRFNASLGTYH |
| | DLLKIIKDKDFLDNEENEDILEDIVLTLTLFEDREMIEERLKTYA |
| | HLEDDKVMKQLKRRRYTGWGRLSRKLINGIRDKQSGKTILDFLKS |
| | DGFANRNFMQLIHDDSLTFKEDIQKAQVSGQGDSLHEHIANLAGS |
| | PAIKKGILQTVKVVDELVKVMGRHIKPENIVIEMARENQTTQKGQ |
| | KNSRERMKRIEEGIKELGSQILKEHPVENTQLQNEKLYLYYLQNG |
| | RDMYVDQELDINRLSDYDVDAIVPQSFLKDDSIDNKVLTRSDKNR |
| | GKSDNVPSEEVVKKMKNYWRQLLNAKLITQRKFDNLTKAERGGLS |
| | ELDKAGFIKRQLVETRQITKHVAQILDSRMNTKYDENDKLIREVK |
| | VITLKSKLVSDERKDFQFYKVREINNYHHAHDAYLNAVVGTALIK |
| | KYPKLESEFVYGDYKVYDVRKMIAKSEQEIGKATAKYFFYSNIMN |
| | FFKTEITLANGEIRKRPLIETNGETGEIVWDKGRDFATVRKVLSM |
| | PQVNIVKKTEVQTGGFSKESILPKRNSDKLIARKKDWDPKKYGGF |
| | DSPTVAYSVLVVAKVEKGKSKKLKSVKELLGITIMERSSFEKNPI |
| | DFLEAKGYKEVKKDLIIKLPKYSLFELENGRKRMLASAGELQKGN |
| | ELALPSKYVNFLYLASHYEKLKGSPEDNEQKQLEVEQHKHYLDEI |
| | IEQISEFSKRVILADANLDKVLSAYNKHRDKPIREQAENIIHLFT |
| | LINLGAPAAFKYEDTTIDRKRYTSTKEVLDATLIHQSITGLYETR |
| | IDLSQLGGDSGGSKRTADGSEFEPKKKRKV |
| | (SEQ ID NO: 109) |
| | key |
| | Nuclear localization sequence (NLS) Top:(SEQ |
| | ID NO: 101), |
| | Bottom: (SEQ ID NO: 103) |
| | Cas9(H840A)(SEQ ID NO: 37) |
| | amino acid linker (SEQ ID NO: 128) |
| | M-MLV reverse transcriptase (SEQ ID NO: 81) |
| PE FUSION PROTEIN | MKRTADGSEFESPKKKRKVDKKYSIGLDIGTNSVGWAVITDEYKV |
| CAS9(H840A)-FEN1- | PSKKFKVLGNTDRHSIKKNLIGALLFDSGETAEATRLKRTARRRY |
| MMLV_RT | TRRKNRICYLQEIFSNEMAKVDDSFFHRLEESFLVEEDKKHERHP |
| D200N | IFGNIVDEVAYHEKYPTIYHLRKKLVDSTDKADLRLIYLALAHMI |
| T330P | KFRGHFLIEGDLNPDNSDVDKLFIQLVQTYNQLFEENPINASGVD |
| L603W | AKAILSARLSKSRRLENLIAQLPGEKKNGLFGNLIALSLGLTPNF |
| T306K | KSNFDLAEDAKLQLSKDTYDDDLDNLLAQIGDQYADLFLAAKNLS |
| W313F | DAILLSDILRVNTEITKAPLSASMIKRYDEHHQDLTLLKALVRQQ |
| | LPEKYKEIFFDQSKNGYAGYIDGGASQEEFYKFIKPILEKMDGTE |
| | ELLVKLNREDLLRKQRTFDNGSIPHQIHILGELHAILRRQEDFYP |
| | FLKDNREKIEKILTFRIPYYVGPLARGNSRFAWMTRKSEETITPW |
| | NFEEVVDKGASAQSFIERMTNFDKNLPNEKVLPKHSLLYEYFTVY |
| | NELTKVKYVTEGMRKPAFLSGEQKKAIVDLLFKTNRKVTVKQLKE |
| | DYFKKIECFDSVEISGVEDRENASLGTYHDLLKIIKDKDFLDNEE |
| | NEDILEDIVLTLTLFEDREMIEERLKTYAHLEDDKVMKQLKRRRY |
| | TGWGRLSRKLINGIRDKQSGKTILDELKSDGFANRNFMQLIHDDS |
| | LTFKEDIQKAQVSGQGDSLHEHIANLAGSPAIKKGILQTVKVVDE |
| | LVKVMGRHKPENIVIEMARENQTTQKGQKNSRERMKRIEEGIKEL |
| | GSQILKEHPVENTQLQNEKLYLYYLQNGRDMYVDQELDINRLSDY |
| | DVDAIVPQSFLKDDSIDNKVLTRSDKNRGKSDNVPSEEVVKKMKN |
| | YWRQLLNAKLITQRKEDNLTKAERGGLSELDKAGFIKRQLVETRQ |
| | ITKHVAQILDSRMNTKYDENDKLIREVKVITLKSKLVSDFRKDFQ |
| | FYKVREINNYHHAHDAYLNAVVGTALIKKYPKLESEFVYGDYKVY |
| | DVRKMIAKSEQEIGKATAKYFFYSNIMNFFKTEITLANGEIRKRP |
| | LIETNGETGEIVWDKGRDFATVRKVLSMPQVNIVKKTEVQTGGFS |
| | KESILPKRNSDKLIARKKDWDPKKYGGFDSPTVAYSVLVVAKVEK |
| | GKSKKLKSVKELLGITIMERSSFEKNPIDFLEAKGYKEVKKDLII |
| | KLPKYSLFELENGRKRMLASAGELQKGNELALPSKYVNFLYLASH |
| | YEKLKGSPEDNEQKQLEVEQHKHYLDEHIEQISEFSKRVILADAN |
| | LDKVLSAYNKHRDKPIREQAENHIHLFTLTNLGAPAAFKYFDTTI |
| | DRKRYTSTKEVLDATLIHQSITGLYETRIDLSQLGGDSGGSSGGS |
| | SGSETPGTSESATPESSGGSSGGSSGIQGLAKLIADVAPSAIREN |
| | DIKSYFGRKVAIDASMSIYQFLIAVRQGGDVLQNEEGETTSHLMG |
| | MFYRTIRMMENGIKPVYVFDGKPPQLKSGELAKRSERRAEAEKQL |
| | QQAQAAGAEQEVEKFTKRLVKVTKQHNDECKHLLSLMGIPYLDAP |
| | SEAEASCAALVKAGKVYAAATEDMDCLTFGSPVLMRHLTASEAKK |
| | LPIQEFHLSRILQELGLNQEQFVDLCILLGSDYCESIRGIGPKRA |
| | VDLIQKHKSIEEIVRRLDPNKYPVPENWLHKEAHQLFLEPEVLDP |
| | ESVELKWSEPNEEELIKFMCGEKQFSEERIRSGVKRLSKSRQGST |
| | QGRLDDFFKVTGSLSSAKRKEPEPKGSTKKKAKTGAAGKFKRGKS |
| | GGSSGGSSGSETPGTSESATPESSGGSSGGSSTLNIEDEYRLHET |
| | SKEPDVSLGSTWLSDFPQAWAETGGMGLAVRQAPLIIPLKATSTP |
| | VSIKQYPMSQEARLGIKPHIQRLLDQGILVPCQSPWNTPLLPVKK |
| | PGTNDYRPVQDLREVNKRVEDIHPTVPNPYNLLSGLPPSHQWYTV |
| | LDLKDAFFCLRLHPTSQPLFAFEWRDPEMGISGQLTWTRLPQGFK |
| | NSPTLFNEALHRDLADFRIQHPDLILLQYVDDLLLAATSELDCQQ |
| | GTRALLQTLGNLGYRASAKKAQICQKQVKYLGYLLKEGQRWLTEA |
| | RKETVMGQPTPKTPRQLREFLGKAGFCRLFIPGFAEMAAPLYPLT |
| | KPGTLFNWGPDQQKAYQEIKQALLTAPALGLPDLTKPFELFVDEK |
| | QGYAKGVLTQKLGPWRRPVAYLSKKLDPVAAGWPPCLRMVAAIAV |
| | LTKDAGKLTMGQPLVILAPHAVEALVKQPPDRWLSNARMTHYQAL |
| | LLDTDRVQFGPVVALNPATLLPLPEEGLQHNCLDILAEAHGTRPD |
| | LTDQPLPDADHTWYTDGSSLLQEGQRKAGAAVTTETEVIWAKALP |
| | AGTSAQRAELIALTQALKMAEGKKLNVYTDSRYAFATAHIHGEIY |
| | RRRGWLTSEGKEIKNKDEILALLKALFLPKRLSIIHCPGHQKGHS |
| | AEARGNRMADQAARKAAITETPDTSTLLIENSSPSGGSKRTADGS |
| | EFEPKKKRKV |
| | (SEQ ID NO: 110) |
| | key: |
| | Nuclear localization sequence (NLS) Top:(SEQ |
| | ID NO: 101), |
| | Bottom: (SEQ ID NO: 103) |
| | Cas9(H840A) (SEQ ID NO: 37) |
| | 33-amino acid linker 1 (SEQ ID NO: 102) |
| | M-MLV reverse transcriptase (SEQ ID NO: 98) |
| | 33-amino acid linker 2 (SEQ ID NO: 102) |
| | FEN1 (SEQ ID NO: 113) |

In other embodiments, the prime editor fusion proteins can be based on SaCas9 or on SpCas9 nickases with altered PAM specificities, such as the following exemplary sequences:

| | |
|---|---|
| SACAS9-M-MLV RT | MKRTADGSEFESPKKKRKVGKRNYILGLDIGITSVGYGIIDYETR |
| PRIME EDITOR | DVIDAGVRLFKEANVENNEGRRSKRGARRLKRRRRHRIQRVKKLL |
| | FDYNLLTDHSELSGINPYEARVKGLSQKLSEEEFSAALLHLAKRR |
| | GVHNVNEVEEDTGNELSTKEQISRNSKALEEKYVAELQLERLKKD |
| | GEVRGSINRFKTSDYVKEAKQLLKVQKAYHQLDQSFIDTYIDLLE |
| | TRRTYYEGPGEGSPFGWKDIKEWYEMLMGHCTYFPEELRSVKYAY |
| | NADLYNALNDLNNLVITRDENEKLEYYEKFQIIENVFKQKKKPTL |
| | KQIAKEILVNEEDIKGYRVTSTGKPEFTNLKVYHDIKDITARKED |
| | ENAELLDQIAKILTIYQSSEDIQEELTNLNSELTQEEIEQISNLK |
| | GYTGTHNLSLKAINLILDELWHTNDNQIAIFNRLKLVPKKVDLSQ |
| | QKEIPTTLVDDFILSPVVKRSFIQSIKVINAIIKKYGLPNDIIIE |
| | LAREKNSKDAQKMINEMQKRNRQTNERIEEIIRTTGKENAKYLIE |
| | KIKLHDMQEGKCLYSLEAIPLEDLLNNPFNYEVDHIPRSVSFDNS |
| | FNNKVLVKQEEASKKGNRTPFQYLSSSDSKISYETFKKHILNLAK |
| | GKGRISKTKKEYLLEERDINRFSVQKDFINRNLVDTRYATRGLMN |
| | LLRSYFRVNNLDVKVKSINGGFTSFLRRKWKFKKERNKGYKHHAE |
| | DALIIANADFIFKEWKKLDKAKKVMENQMFEEKQAESMPEIETEQ |
| | EYKEIFITPHQIKHIKDFKDYKYSHRVDKKPNRELINDTLYSTRK |
| | DDKGNTLIVNNLNGLYDKDNDKLKKLINKSPEKLLMYHHDPQTYQ |
| | KLKLIMEQYGDEKNPLYKYYEETGNYLTKYSKKDNGPVIKKIKYY |
| | GNKLNAHLDITDDYPNSRNKVVKLSLKPYRFDVYLDNGVYKFVTV |
| | KNLDVIKKENYYEVNSKCYEEAKKLKKISNQAEFIASFYNNDLIK |
| | INGELYRVIGVNNDLLNRIEVNMIDITYREYLENMNDKRPPRIIK |
| | TIASKTQSIKKYSTDILGNLYEVKSKKHPQIIKKGSGGSSGGSSG |
| | SETPGTSESATPESSGGSSGGSSTLNIEDEYRLHETSKEPDVSLG |
| | STWLSDFPQAWAETGGMGLAVRQAPLIIPLKATSTPVSIKQYPMS |
| | QEARLGIKPHIQRLLDQGILVPCQSPWNTPLLPVKKPGTNDYRPV |
| | QDLREVNKRVEDIHPTVPNPYNLLSGLPPSHQWYTVLDLKDAFFC |
| | LRLHPTSQPLFAFEWRDPEMGISGQLTWTRLPQGFKNSPTLFDEA |
| | LHRDLADFRIQHPDLILLQYVDDLLLAATSELDCQQGTRALLQTL |
| | GNLGYRASAKKAQICQKQVKYLGYLLKEGQRWLTEARKETVMGQP |
| | TPKTPRQLREFLGTAGFCRLWIPGFAEMAAPLYPLTKTGTLFNWG |
| | PDQQKAYQEIKQALLTAPALGLPDLTKPFELFVDEKQGYAKGVLT |
| | QKLGPWRRPVAYLSKKLDPVAAGWPPCLRMVAAIAVLTKDAGKLT |
| | MGQPLVILAPHAVEALVKQPPDRWLSNARMTHYQALLLDTDRVQF |
| | GPVVALNPATLLPLPEEGLQHNCLDILAEAHGTRPDLTDQPLPDA |
| | DHTWYTDGSSLLQEGQRKAGAAVTTETEVIWAKALPAGTSAQRAE |
| | LIALTQALKMAEGKKLNVYTDSRYAFATAHIHGEIYRRRGLLTSE |
| | GKEIKNKDEILALLKALFLPKRLSIIHCPGHQKGHSAEARGNRMA |
| | DQAARKAAITETPDTSTLLIENSSPSGGSKRTADGSEFEPKKKRK |
| | V |
| | (SEQ ID NO: 114) |
| SPCAS9(H840A)- | MKRTADGSEFESPKKKRKVDKKYSIGLDIGTNSVGWAVITDEYKV |
| VRQR-MALONEY | PSKKFKVLGNTDRHSIKKNLIGALLFDSGETAEATRLKRTARRRY |
| MURINE LEUKEMIA | TRRKNRICYLQEIFSNEMAKVDDSFFHRLEESFLVEEDKKHERHP |
| VIRUS REVERSE | IFGNIVDEVAYHEKYPTIYHLRKKLVDSTDKADLRLIYLALAHMI |
| TRANSCRIPTASE | QKFRGHFLIEGDLNPDNSDVDKLFIQLVQTYNLFEENPINASGVD |
| PRIME EDITOR | AKAILSARLSKSRRLENLIAQLPGEKKNGLFGNLIALSLGLTPNF |
| | AKSNFDLAEDAKLQLSKDTYDDDLDNLLAQIGDQYADLFLAAKNL |
| | SDILLSDILRVNTEITKAPLSASMIKRYDEHHQDLILLKALVRQQ |
| | LPEKYKEIFFDQSKNGYAGYIDGGASQEEFYKFIKPILEKMDGTE |
| | ELLVKLNREDLLRKQRTFDNGSIPHQIHLGELHAILRRQEDFYPF |
| | LKDNREKIEKILTFRIPYYVGPLARGNSRFAWMTRKSEETITPWN |
| | FEEVVDKGASAQSFIERMTNFDKNLPNEKVLPKHSLLYEYFTVYN |
| | ELTKVKYVTEGMRKPAFLSGEQKKAIVDLLFKTNRKVTVKQLKED |
| | YFKKIECFDSVEISGVEDRFNASLGTYHDLLKIIKDKDFLDNEEN |
| | EDILEDIVLTLTLFEDREMIEERLKTYAHLEDDKVMKQLKRRRYT |
| | GWGRLSRKLINGIRDKQSGKTILDFLKSDGFANRNFMQLIHDDSL |
| | TFKEDIQKAQVSGQGDSLHEHIANLAGSPAIKKGILQTVKVVDEL |
| | VKVMGRHKPENIVIEMARENQTTQKGQKNSRERMKRIEEGIKELG |
| | SQILKEHPVENTQLQNEKLYLYYLQNGRDMYVDQELDINRLSDYD |
| | VDAIVPQSFLKDDSIDNKVLTRSDKNRGKSDNVPSEEVVKKMKNY |
| | WRQLLNAKLITQRKEDNLTKAERGGLSELDKAGFIKRQLVETRQI |
| | TKHVAQILDSRMNTKYDENDKLIREVKVITLKSKLVSDFRKDFQF |
| | YKVREINNYHHAHDAYLNAVVGTALIKKYPKLESEFVYGDYKVYD |
| | VRKMIAKSEQEIGKATAKYFFYSNIMNFFKTEITLANGEIRKRPL |
| | IETNGETGEIVWDKGRDFATVRKVLSMPQVNIVKKTEVQTGGFSK |
| | ESILPKRNSDKLIARKKDWDPKKYGGFVSPTVAYSVLVVAKVEKG |
| | KSKKLKSVKELLGITIMERSSFEKNPIDFLEAKGYKEVKKDLIIK |
| | LPKYSLFELENGRKRMLASARELQKGNELALPSKYVNFLYLASHY |
| | EKLKGSPEDNEQKQLFVEQHKHYLDEIIEQISEFSKRVILADANL |
| | DKVLSAYNKHRDKPIREQAENIIHLFTLTNLGAPAAFKYFDTTID |
| | RKQYRSTKEVLDATLIHQSITGLYETRIDLSQLGGDSGGSSGGSS |
| | GSETPGTSESATPESSGGSSGGSSTLNIEDEYRLHETSKEPDVSL |
| | GSTWLSDFPQAWAETGGMGLAVRQAPLIIPLKATSTPVSIKQYPM |
| | SQEARLGIKPHIQRLLDQGILVPCQSPWNTPLLPVKKPGTNDYRP |
| | VQDLREVNKRVEDIHPTVPNPYNLLSGLPPSHQWYTVLDLKDAFF |
| | CLRLHPTSQPLFAFEWRDPEMGISGQLTWTRLPQGFKNSPTLFNE |
| | ALHRDLADFRIQHPDLILLQYVDDLLLAATSELDCQQGTRALLQT |
| | LGNLGYRASAKKAQICQKQVKYLGYLLKEGQRWLTEARKETVMGQ |
| | PTPKTPRQLREFLGKAGFCRLFIPGFAEMAAPLYPLTKPGTLFNW |
| | GPDQQKAYQEIKQALLTAPALGLPDLTKPFELFVDEKQGYAKGVL |
| | TQKLGPWRRPVAYLSKKLDPVAAGWPPCLRMVAAIAVLTKDAGKL |
| | TMGQPLVILAPHAVEALVKQPPDRWLSNARMTHYQALLLDTDRVQ |
| | FGPVVALNPATLLPLPEEGLQHNCLDILAEAHGTRPDLTDQPLPD |
| | ADHTWYTDGSSLLQEGQRKAGAAVTTETEVIWAKALPAGTSAQRA |
| | ELIALTQALKMAEGKKLNVYTDSRYAFATAHIHGEIYRRRGWLTS |
| | EGKEIKNKDEILALLKALFLPKRLSIIHCPGHQKGHSAEARGNRM |
| | ADQAARKAAITETPDTSTLLIENSSPSGGSKRTADGSEFEPKKKR |
| | KV |
| | (SEQ ID NO: 115) |
| SPCAS9(H840A)- | MKRTADGSEFESPKKKRKVDKKYSIGLDIGTNSVGWAVITDEYKV |
| VRER-MALONEY | PSKKFKVLGNTDRHSIKKNLIGALLFDSGETAEATRLKRTARRRY |
| MURINE LEUKEMIA | TRRKNRICYLQEIFSNEMAKVDDSFFHRLEESFLVEEDKKHERHP |
| VIRUS REVERSE | IFGNIVDEVAYHEKYPTIYHLRKKLVDSTDKADLRLIYLALAHMI |
| TRANSCRIPTASE | KFRGHFLIEGDLNPDNSDVDKLFIQLVQTYNQLFEENPINASGVD |
| PRIME EDITOR | AKAILSARLSKSRRLENLIAQLPGEKKNGLFGNLIALSLGLTPNF |
| | KSNFDLAEDAKLQLSKDTYDDDLDNLLAQIGDQYADLFLAAKNLS |
| | DAILLSDILRVNTEITKAPLSASMIKRYDEHHQDLTLLKALVRQQ |
| | LPEKYKEIFFDQSKNGYAGYIDGGASQEEFYKFIKPILEKMDGTE |
| | ELLVKLNREDLLRKQRTFDNGSIPHQIHLGELHAILRRQEDFYPF |
| | LKDNREKIEKILTFRIPYYVGPLARGNSRFAWMTRKSEETITPWN |
| | FEEVVDKGASAQSFIERMTNFDKNLPNEKVLPKHSLLYEYFTVYN |
| | ELTKVKYVTEGMRKPAFLSGEQKKAIVDLLFKTNRKVTVKQLKED |
| | YFKKIECFDSVEISGVEDRFNASLGTYHDLLKIIKDKDFLDNEEN |
| | EDILEDIVLTLTLFEDREMIEERLKTYAHLEDDKVMKQLKRRRYT |
| | GWGRLSRKLINGIRDKQSGKTILDFLKSDGFANRNFMQLIHDDSL |
| | TFKEDIQKAQVSGQGDSLHEHIANLAGSPAIKKGILQTVKVVDEL |
| | VKVMGRHKPENIVIEMARENQTTQKGQKNSRERMKRIEEGIKELG |
| | SQILKEHPVENTQLQNEKLYLYYLQNGRDMYVDQELDINRLSDYD |
| | VDAIVPQSFLKDDSIDNKVLTRSDKNRGKSDNVPSEEVVKKMKNY |
| | WRQLLNAKLITQRKFDNLTKAERGGLSELDKAGFIKRQLVETRQI |
| | TKHVAQILDSRMNTKYDENDKLIREVKVITLKSKLVSDFRKDFQF |
| | YKVREINNYHHAHDAYLNAVVGTALIKKYPKLESEFVYGDYKVYD |
| | VRKMIAKSEQEIGKATAKYFFYSNIMNFFKTEITLANGEIRKRPL |
| | IETNGETGEIVWDKGRDFATVRKVLSMPQVNIVKKTEVQTGGFSK |
| | ESILPKRNSDKLIARKKDWDPKKYGGFVSPTVAYSVLVVAKVEKG |
| | KSKKLKSVKELLGITIMERSSFEKNPIDFLEAKGYKEVKKDLIKL |
| | PKYSLFELENGRKRMLASARELQKGNELALPSKYVNFLYLASHYE |
| | KLKGSPEDNEQKQLFVEQHKHYLDEHEQISEFSKRVILADANLDK |
| | VLSAYNKHRDKPIREQAENIIHLFTLTNLGAPAAFKYFDTTIDRK |
| | EYRSTKEVLDATLIHQSITGLYETRIDLSQLGGDSGGSSGGSSGS |
| | ETPGTSESATPESSGGSSGGSSTLNIEDEYRLHETSKEPDVSLGS |
| | TWLSDFPQAWAETGGMGLAVRQAPLIIPLKATSTPVSIKQYPMSQ |
| | EARLGIKPHIQRLLDQGILVPCQSPWNTPLLPVKKPGTNDYRPVQ |
| | DLREVNKRVEDIHPTVPNPYNLLSGLPPSHQWYTVLDLKDAFFCL |
| | RLHPTSQPLFAFEWRDPEMGISGQLTWTRLPQGFKNSPTLFNEAL |
| | HRDLADFRIQHPDLILLQYVDDLLLAATSELDCQQGTRALLQTLG |
| | NLGYRASAKKAQICQKQVKYLGYLLKEGQRWLTEARKETVMGQPT |
| | PKTPRQLREFLGKAGFCRLFIPGFAEMAAPLYPLTKPGTLFNWGP |
| | DQQKAYQEIKQALLTAPALGLPDLTKPFELFVDEKQGYAKGVLTQ |
| | KLGPWRRPVAYLSKKLDPVAAGWPPCLRMVAAIAVLTKDAGKLTM |
| | GQPLVILAPHAVEALVKQPPDRWLSNARMTHYQALLLDTDRVQFG |
| | PVVALNPATLLPLPEEGLQHNCLDILAEAHGTRPDLTDQPLPDAD |
| | HTWYTDGSSLLQEGQRKAGAAVTTETEVIWAKALPAGTSAQRAEL |
| | IALTQALKMAEGKKLNVYTDSRYAFATAHIHGEIYRRRGWLTSEG |
| | KEIKNKDEILALLKALFLPKRLSIIHCPGHQKGHSAEARGNRMAD |
| | QAARKAAITETPDTSTLLIENSSPSGGSKRTADGSEFEPKKKRKV |
| | (SEQ ID NO: 116) |

In yet other embodiments, the prime editor fusion proteins utilized in the methods and compositions contemplated herein may include a Cas9 nickase (e.g., Cas9 (H840A)) fused to a truncated version of M-MLV reverse transcriptase. In this embodiment, the reverse transcriptase also contains 4 mutations (D200N. T306K, W313F, T330P; noting that the L603W mutation present in PE2 is no longer present due to the truncation). The DNA sequence encoding this truncated editor is 522 bp smaller than PE2, and therefore makes its potentially useful for applications where delivery of the DNA sequence is challenging due to its size (i.e., adeno-associated virus and lentivirus delivery). This embodiment is referred to as Cas9(H840A)-MMLV-RT(trunc) or “PE2-short” or “PE2-trune” and has the following amino acid sequence:

| | |
|---|---|
| CAS9(H840A)- | MKRTADGSEFESPKKKRKVDKKYSIGLDIGTNSVGWAVITDEYKVPSKKF |
| MMLV-RT(TRUNC) | KVLGNTDRHSIKKNLIGALLFDSGETAEATRLKRTARRRYTRRKNRICYL |
| OR PE2-SHORT | QEIFSNEMAKVDDSFFHRLEESFLVEEDKKHERHPIFGNIVDEVAYHEKY |
| | PTIYHLRKKLVDSTDKADLRLIYLALAHMIKERGHFLIEGDLNPDNSDVD |
| | KLFIQLVQTYNQLFEENPINASGVDAKAILSARLSKSRRLENLIAQLPGE |
| | KKNGLFGNLIALSLGLTPNFKSNFDLAEDAKLQLSKDTYDDDLDNLLAQI |
| | GDQYADLFLAAKNLSDAILLSDILRVNTEITKAPLSASMIKRYDEHHQDL |
| | TLLKALVRQQLPEKYKEIFFDQSKNGYAGYIDGGASQEEFYKFIKPILEK |
| | MDGTEELLVKLNREDLLRKQRTFDNGSIPHQIHLGELHAILRRQEDFYPF |
| | LKDNREKIEKILTFRIPYYVGPLARGNSRFAWMTRKSEETITPWNFEEVV |
| | DKGASAQSFIERMTNFDKNLPNEKVLPKHSLLYEYFTVYNELTKVKYVTE |
| | GMRKPAFLSGEQKKAIVDLLFKTNRKVTVKQLKEDYFKKIECFDSVEISG |
| | VEDRFNASLGTYHDLLKIIKDKDELDNEENEDILEDIVLTLTLFEDREMI |
| | EERLKTYAHLEDDKVMKQLKRRRYTGWGRLSRKLINGIRDKQSGKTILDE |
| | LKSDGFANRNFMQLIHDDSLTFKEDIQKAQVSGQGDSLHEHIANLAGSPA |
| | IKKGILQTVKVVDELVKVMGRHKPENIVIEMARENQTTQKGQKNSRERMK |
| | RIEEGIKELGSQILKEHPVENTQLQNEKLYLYYLQNGRDMYVDQELDINR |
| | LSDYDVDAIVPQSFLKDDSIDNKVLTRSDKNRGKSDNVPSEEVVKKMKNY |
| | WRQLLNAKLITQRKFDNLTKAERGGLSELDKAGFIKRQLVETRQITKHVA |
| | QILDSRMNTKYDENDKLIREVKVITLKSKLVSDFRKDFQFYKVREINNYH |
| | HAHDAYLNAVVGTALIKKYPKLESEFVYGDYKVYDVRKMIAKSEQEIGKA |
| | TAKYFFYSNIMNFFKTEITLANGEIRKRPLIETNGETGEIVWDKGRDFAT |
| | VRKVLSMPQVNIVKKTEVQTGGFSKESILPKRNSDKLIARKKDWDPKKYG |
| | GFDSPTVAYSVLVVAKVEKGKSKKLKSVKELLGITIMERSSFEKNPIDFL |
| | EAKGYKEVKKDLIIKLPKYSLFELENGRKRMLASAGELQKGNELALPSKY |
| | VNFLYLASHYEKLKGSPEDNEQKQLFVEQHKHYLDEHIEQISEFSKRVIL |
| | ADANLDKVLSAYNKHRDKPIREQAENIIHLFTLTNLGAPAAFKYFDTTID |
| | RKRYTSTKEVLDATLIHQSITGLYETRIDLSQLGGDSGGSSGGSSGSETP |
| | GTSESATPESSGGSSGGSSTLNIEDEYRLHETSKEPDVSLGSTWLSDFPQ |
| | AWAETGGMGLAVRQAPLIIPLKATSTPVSIKQYPMSQEARLGIKPHIQRL |
| | LDQGILVPCQSPWNTPLLPVKKPGTNDYRPVQDLREVNKRVEDIHPTVPN |
| | PYNLLSGLPPSHQWYTVLDLKDAFFCLRLHPTSQPLFAFEWRDPEMGISG |
| | QLTWTRLPQGFKNSPTLFNEALHRDLADFRIQHPDLILLQYVDDLLLAAT |
| | SELDCQQGTRALLQTLGNLGYRASAKKAQICQKQVKYLGYLLKEGQRWLT |
| | EARKETVMGQPTPKTPRQLREFLGKAGFCRLFIPGFAEMAAPLYPLTKPG |
| | TLFNWGPDQQKAYQEIKQALLTAPALGLPDLTKPFELFVDEKQGYAKGVL |
| | TQKLGPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVALNPA |
| | TLLPLPEEGLQHNCLDNSRLINSGGSKRTADGSEFEPKKKRKV |
| | (SEQ ID NO: 117) |
| | KEY: |
| | NUCLEAR LOCALIZATION SEQUENCE (NLS) TOP: |
| | (SEQ ID NO: 101), |
| | BOTTOM: (SEQ ID NO: 103) |
| | CAS9(H840A) (SEQ ID NO: 37) |
| | 33-AMINO ACID LINKER 1 (SEQ ID NO: 102) |
| | M-MLV TRUNCATED reverse transcriptase |
| | (SEQ ID NO: 80) |

In various embodiments, the prime editor fusion proteins utilized in the methods and compositions contemplated herein may also include any variants of the above-disclosed sequences having an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to PE1, PE2, or any of the above indicated prime editor fusion sequences.

In certain embodiments, linkers may be used to link any of the peptides or peptide domains or moieties of the invention (e.g., a napDNAbp linked or fused to a reverse transcriptase).

### Prime Editors: Modified PE Fusion Proteins (e.g., PEmax)

In one aspect, the present disclosure provides modified prime editor proteins. In one embodiment, the modified prime editor fusion protein is PEmax (of SEQ ID NO: 99), or an amino acid sequence having at least 80%, at least 85%, at least 90%, at least 95%, at least 96%, at least 97%, at least 98%, at least 99%, or at least up to 100% sequence identify with SEQ ID NO: 99. The sequence of PEmax is as follows:

KEY:
Bipartite SV40 NLS
SpCas9 R221K N394K H840A
Linker = (SGGSx2-bipartite SV40 NLS-SGGSx2)
(SGGSSGGS KRTADGSEFESPKKKRKV SGGSSGGS) (SEQ ID NO: 105)
Genscript codon optimized MMLV RT pentamutant
(D200N T306K W313F T330P L603W)
Other linker sequences
c-Myc NLS
PEmax protein sequence (SEQ ID NO: 99):
MKRTADGSEFESPKKKRKVDKKYSIGLDIGTNSVGWAVITDEYKVPSKKFKVLGNTDRHS
IKKNLIGALLFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRKL
ENLIAQLPGEKKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVKLKREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYN
ELTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDM
YVDQELDINRLSDYDVDAIVPQSFLKDDSIDNKVLTRSDKNRGKSDNVPSEEVVKKMKNY
WRQLLNAKLITQRKEDNLTKAERGGLSELDKAGFIKRQLVETRQITKHVAQILDSRMNTK
YDENDKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLANGEIRKRPL
IETNGETGEIVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKYVNFLYLASHY
EKLKGSPEDNEQKQLFVEQHKHYLDEIEQISEFSKRVILADANLDKVLSAYNKHRDKPIR
EQAENIIHLFTLTNLGAPAAFKYFDTTIDRKRYTSTKEVLDATLIHQSITGLYETRIDLS
QLGGDSGGSSGGSKRTADGSEFESPKKKRKVSGGSSGGSTLNIEDEYRLHETSKEPDVSL
GSTWLSDFPQAWAETGGMGLAVRQAPLIIPLKATSTPVSIKQYPMSQEARLGIKPHIQRL
LDQGILVPCQSPWNTPLLPVKKPGTNDYRPVQDLREVNKRVEDIHPTVPNPYNLLSGLPP
SHQWYTVLDLKDAFFCLRLHPTSQPLFAFEWRDPEMGISGQLTWTRLPQGFKNSPTLFNE
ALHRDLADFRIQHPDLILLQYVDDLLLAATSELDCQQGTRALLQTLGNLGYRASAKKAQI
CQKQVKYLGYLLKEGQRWLTEARKETVMGQPTPKTPRQLREFLGKAGFCRLFIPGFAEMA
APLYPLTKPGTLFNWGPDQQKAYQEIKQALLTAPALGLPDLTKPFELFVDEKQGYAKGVL
TQKLGPWRRPVAYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEGLQHNCLDILAE
AHGTRPDLTDQPLPDADHTWYTDGSSLLQEGQRKAGAAVTTETEVIWAKALPAGTSAQRA
ELIALTQALKMAEGKKLNVYTDSRYAFATAHIHGEIYRRRGWLTSEGKEIKNKDEILALL
KALFLPKRLSIIHCPGHQKGHSAEARGNRMADQAARKAAITETPDTSTLLIENSSPSGGS
KRTADGSEFESPKKKRKVGSGPAAKRVKLD
PEmax DNA sequence (SEQ ID NO: 5660):
ATGAAACGGACAGCCGACGGAAGCGAGTTCGAGTCACCAAAGAAGAAGCGGAAAGTCGAC
AAGAAGTACAGCATCGGCCTGGACATCGGCACCAACTCTGTGGGCTGGGCCGTGATCACC
GACGAGTACAAGGTGCCCAGCAAGAAATTCAAGGTGCTGGGCAACACCGACCGGCACAGC
ATCAAGAAGAACCTGATCGGAGCCCTGCTGTTCGACAGCGGCGAAACAGCCGAGGCCACC
CGGCTGAAGAGAACCGCCAGAAGAAGATACACCAGACGGAAGAACCGGATCTGCTATCTG
CAAGAGATCTTCAGCAACGAGATGGCCAAGGTGGACGACAGCTTCTTCCACAGACTGGAA
GAGTCCTTCCTGGTGGAAGAGGATAAGAAGCACGAGCGGCACCCCATCTTCGGCAACATC
GTGGACGAGGTGGCCTACCACGAGAAGTACCCCACCATCTACCACCTGAGAAAGAAACTG
GTGGACAGCACCGACAAGGCCGACCTGCGGCTGATCTATCTGGCCCTGGCCCACATGATC
AAGTTCCGGGGCCACTTCCTGATCGAGGGCGACCTGAACCCCGACAACAGCGACGTGGAC
AAGCTGTTCATCCAGCTGGTGCAGACCTACAACCAGCTGTTCGAGGAAAACCCCATCAAC
GCCAGCGGCGTGGACGCCAAGGCCATCCTGTCTGCCAGACTGAGCAAGAGCAGAAAGCTG
GAAAATCTGATCGCCCAGCTGCCCGGCGAGAAGAAGAATGGCCTGTTCGGAAACCTGATT
GCCCTGAGCCTGGGCCTGACCCCCAACTTCAAGAGCAACTTCGACCTGGCCGAGGATGCC
AAACTGCAGCTGAGCAAGGACACCTACGACGACGACCTGGACAACCTGCTGGCCCAGATC
GGCGACCAGTACGCCGACCTGTTTCTGGCCGCCAAGAACCTGTCCGACGCCATCCTGCTG
AGCGACATCCTGAGAGTGAACACCGAGATCACCAAGGCCCCCCTGAGCGCCTCTATGATC
AAGAGATACGACGAGCACCACCAGGACCTGACCCTGCTGAAAGCTCTCGTGCGGCAGCAG
CTGCCTGAGAAGTACAAAGAGATTTTCTTCGACCAGAGCAAGAACGGCTACGCCGGCTAC
ATTGACGGCGGAGCCAGCCAGGAAGAGTTCTACAAGTTCATCAAGCCCATCCTGGAAAAG
ATGGACGGCACCGAGGAACTGCTCGTGAAGCTGAAGAGAGAGGACCTGCTGCGGAAGCAG
CGGACCTTCGACAACGGCAGCATCCCCCACCAGATCCACCTGGGAGAGCTGCACGCCATT
CTGCGGCGGCAGGAAGATTTTTACCCATTCCTGAAGGACAACCGGGAAAAGATCGAGAAG
ATCCTGACCTTCCGCATCCCCTACTACGTGGGCCCTCTGGCCAGGGGAAACAGCAGATTC
GCCTGGATGACCAGAAAGAGCGAGGAAACCATCACCCCCTGGAACTTCGAGGAAGTGGTG
GACAAGGGCGCTTCCGCCCAGAGCTTCATCGAGCGGATGACCAACTTCGATAAGAACCTG
CCCAACGAGAAGGTGCTGCCCAAGCACAGCCTGCTGTACGAGTACTTCACCGTGTATAAC
GAGCTGACCAAAGTGAAATACGTGACCGAGGGAATGAGAAAGCCCGCCTTCCTGAGCGGC
GAGCAGAAAAAGGCCATCGTGGACCTGCTGTTCAAGACCAACCGGAAAGTGACCGTGAAG
CAGCTGAAAGAGGACTACTTCAAGAAAATCGAGTGCTTCGACTCCGTGGAAATCTCCGGC
GTGGAAGATCGGTTCAACGCCTCCCTGGGCACATACCACGATCTGCTGAAAATTATCAAG
GACAAGGACTTCCTGGACAATGAGGAAAACGAGGACATTCTGGAAGATATCGTGCTGACC
CTGACACTGTTTGAGGACAGAGAGATGATCGAGGAACGGCTGAAAACCTATGCCCACCTG
TTCGACGACAAAGTGATGAAGCAGCTGAAGCGGCGGAGATACACCGGCTGGGGCAGGCTG
AGCCGGAAGCTGATCAACGGCATCCGGGACAAGCAGTCCGGCAAGACAATCCTGGATTTC
CTGAAGTCCGACGGCTTCGCCAACAGAAACTTCATGCAGCTGATCCACGACGACAGCCTG
ACCTTTAAAGAGGACATCCAGAAAGCCCAGGTGTCCGGCCAGGGCGATAGCCTGCACGAG
CACATTGCCAATCTGGCCGGCAGCCCCGCCATTAAGAAGGGCATCCTGCAGACAGTGAAG
GTGGTGGACGAGCTCGTGAAAGTGATGGGCCGGCACAAGCCCGAGAACATCGTGATCGAA
ATGGCCAGAGAGAACCAGACCACCCAGAAGGGACAGAAGAACAGCCGCGAGAGAATGAAG
CGGATCGAAGAGGGCATCAAAGAGCTGGGCAGCCAGATCCTGAAAGAACACCCCGTGGAA
AACACCCAGCTGCAGAACGAGAAGCTGTACCTGTACTACCTGCAGAATGGGCGGGATATG
TACGTGGACCAGGAACTGGACATCAACCGGCTGTCCGACTACGATGTGGACGCTATCGTG
CCTCAGAGCTTTCTGAAGGACGACTCCATCGACAACAAGGTGCTGACCAGAAGCGACAAG
AACCGGGGCAAGAGCGACAACGTGCCCTCCGAAGAGGTCGTGAAGAAGATGAAGAACTAC
TGGCGGCAGCTGCTGAACGCCAAGCTGATTACCCAGAGAAAGTTCGACAATCTGACCAAG
GCCGAGAGAGOCGGCCTGAGCGAACTGGATAAGGCCGGCTTCATCAAGAGACAGCTGGTG
GAAACCCGGCAGATCACAAAGCACGTGGCACAGATCCTGGACTCCCGGATGAACACTAAG
TACGACGAGAATGACAAGCTGATCCGGGAAGTGAAAGTGATCACCCTGAAGTCCAAGCTG
GTGTCCGATTTCCGGAAGGATTTCCAGTTTTACAAAGTGCGCGAGATCAACAACTACCAC
CACGCCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGATGATC
GCCAAGAGCGAGCAGGAAATCGGCAAGGCTACCGCCAAGTACTTCTTCTACAGCAACATC
ATGAACTTTTTCAAGACCGAGATTACCCTGGCCAACGGCGAGATCCGGAAGCGGCCTCTG
ATCGAGACAAACGGCGAAACCGGGGAGATCGTGTGGGATAAGGGCCGGGATTTTGCCACC
GTGCGGAAAGTGCTGAGCATGCCCCAAGTGAATATCGTGAAAAAGACCGAGGTGCAGACA
GGCGGCTTCAGCAAAGAGTCTATCCTGCCCAAGAGGAACAGCGATAAGCTGATCGCCAGA
AAGAAGGACTGGGACCCTAAGAAGTACGGCGGCTTCGACAGCCCCACCGTGGCCTATTCT
GTGCTGGTGGTGGCCAAAGTGGAAAAGGGCAAGTCCAAGAAACTGAAGAGTGTGAAAGAG
CTGCTGGGGATCACCATCATGGAAAGAAGCAGCTTCGAGAAGAATCCCATCGACTTTCTG
GAAGCCAAGGGCTACAAAGAAGTGAAAAAGGACCTGATCATCAAGCTGCCTAAGTACTCC
CTGTTCGAGCTGGAAAACGGCCGGAAGAGAATGCTGGCCTCTGCCGGCGAACTGCAGAAG
GGAAACGAACTGGCCCTGCCCTCCAAATATGTGAACTTCCTGTACCTGGCCAGCCACTAT
GAGAAGCTGAAGGGCTCCCCCGAGGATAATGAGCAGAAACAGCTGTTTGTGGAACAGCAC
AAGCACTACCTGGACGAGATCATCGAGCAGATCAGCGAGTTCTCCAAGAGAGTGATCCTG
GCCGACGCTAATCTGGACAAAGTGCTGTCCGCCTACAACAAGCACCGGGATAAGCCCATC
AGAGAGCAGGCCGAGAATATCATCCACCTGTTTACCCTGACCAATCTGGGAGCCCCTGCC
GCCTTCAAGTACTTTGACACCACCATCGACCGGAAGAGGTACACCAGCACCAAAGAGGTG
CTGGACGCCACCCTGATCCACCAGAGCATCACCGGCCTGTACGAGACACGGATCGACCTG
TCTCAGCTGGGAGGTGACTCCGGCGGAAGCTCTGGTGGCAGCAAGCGGACCGCCGACGGC
TCTGAATTCGAGAGCCCTAAGAAGAAAAGAAAGGTGAGCGGAGGCTCTAGCGGCGGAAGC
ACCCTGAACATTGAAGACGAGTATAGACTGCATGAAACAAGCAAGGAACCCGACGTGTCC
CTGGGCTCCACCTGGCTGTCCGACTTTCCCCAGGCCTGGGCCGAGACAGGAGGAATGGGC
CTGGCCGTGCGGCAGGCACCCCTGATCATCCCTCTGAAGGCCACCTCTACACCCGTGAGC
ATCAAGCAGTACCCTATGTCTCAGGAGGCCAGACTGGGCATCAAGCCTCACATCCAGAGG
CTGCTGGACCAGGGCATCCTGGTGCCATGCCAGAGCCCCTGGAACACACCACTGCTGCCC
GTGAAGAAGCCAGGCACCAATGACTATAGACCCGTGCAGGATCTGAGAGAGGTGAACAAG
AGGGTGGAGGATATCCACCCCACCGTGCCCAACCCTTACAATCTGCTGTCCGGCCTGCCC
CCTTCTCACCAGTGGTATACAGTGCTGGACCTGAAGGATGCCTTCTTTTGTCTGAGACTG
CACCCTACCAGCCAGCCACTGTTCGCCTTTGAGTGGAGGGACCCTGAGATGGGCATCTCT
GGCCAGCTGACCTGGACACGCCTGCCTCAGGGCTTCAAGAATAGCCCAACACTGTTTAAC
GAGGCCCTGCACCGCGACCTGGCAGATTTCCGGATCCAGCACCCAGATCTGATCCTGCTG
CAGTACGTGGACGATCTGCTGCTGGCCGCCACCAGCGAGCTGGATTGCCAGCAGGGAACA
CGCGCCCTGCTGCAGACCCTGGGAAACCTGGGATATAGGGCATCCGCCAAGAAGGCCCAG
ATCTGTCAGAAGCAGGTGAAGTACCTGGGCTATCTGCTGAAGGAGGGCCAGAGATGGCTG
ACAGAGGCCAGGAAGGAGACAGTGATGGGCCAGCCAACACCCAAGACCCCAAGACAGCTG
AGGGAGTTCCTGGGCAAAGCAGGATTTTGCAGGCTGTTCATCCCAGGATTCGCAGAGATG
GCAGCACCTCTGTACCCACTGACCAAGCCGGGCACCCTGTTTAATTGGGGCCCTGACCAG
CAGAAGGCCTATCAGGAGATCAAGCAGGCCCTGCTGACAGCACCAGCCCTGGGCCTGCCA
GACCTGACCAAGCCTTTCGAGCTGTTTGTGGATGAGAAGCAGGGCTACGCCAAGGGCGTG
CTGACCCAGAAGCTGGGACCATGGAGACGGCCCGTGGCCTATCTGTCCAAGAAGCTGGAC
CCAGTGGCAGCAGGATGGCCACCATGCCTGAGGATGGTGGCAGCAATCGCCGTGCTGACA
AAGGATGCCGGCAAGCTGACCATGGGACAGCCACTGGTCATCCTGGCACCACACGCAGTG
GAGGCCCTGGTGAAGCAGCCTCCAGATCGCTGGCTGTCTAACGCCCGGATGACACACTAC
CAGGCCCTGCTGCTGGACACCGATCGCGTGCAGTTTGGCCCTGTGGTGGCCCTGAATCCA
GCCACCCTGCTGCCTCTGCCAGAGGAGGGCCTGCAGCACAACTGTCTGGACATCCTGGCA
GAGGCACACGGAACAAGGCCAGACCTGACCGATCAGCCCCTGCCTGACGCCGATCACACA
TGGTATACCGATGGAAGCTCCCTGCTGCAGGAGGGCCAGAGGAAGGCAGGAGCAGCAGTG
ACCACAGAGACAGAAGTGATCTGGGCCAAGGCCCTGCCAGCAGGCACATCCGCCCAGCGG
GCCGAGCTGATCGCCCTGACCCAGGCCCTGAAGATGGCCGAGGGCAAGAAGCTGAACGTG
TACACAGACTCCAGATATGCCTTCGCCACCGCACACATCCACGGAGAGATCTACAGGCGC
CGGGGCTGGCTGACCTCTGAGGGCAAGGAGATCAAGAACAAGGATGAGATCCTGGCCCTG
CTGAAGGCCCTGTTTCTGCCCAAGCGGCTGAGCATCATCCACTGTCCTGGACACCAGAAG
GGACACTCCGCCGAGGCAAGGGGCAATCGGATGGCCGACCAGGCCGCCAGAAAGGCTGCT
ATTACTGAAACTCCCGACACTTCCACTCTGCTGATTGAAAACTCCTCCCCTTCTGGCGGC
TCAAAAAGAACCGCCGACGGCAGCGAATTCGAGTCTCCCAAGAAGAAGAGGAAAGTCGGC
TCTGGCCCTGCCGCTAAGAGAGTGAAGCTGGAC
PEmax component sequences of SEQ ID NO: 99:
Bipartite SV40 NLS:
MKRTADGSEFESPKKKRKV (SEQ ID NO: 101)
SpCas9 R221K N394K H840A:
DKKYSIGLDIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRKLENLIAQLPGEKKNGLFGNL
IALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAG
YIDGGASQEEFYKFIKPILEKMDGTEELLVKLKREDLLRKQRTFDNGSIPHQIHLGELHA
ILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQGDSLH
EHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDAI
VPQSFLKDDSIDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGRDFA
TVRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPKY
SLFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGD
(SEQ ID NO: 104)
Linker = (SGGSx2-bipartite SV40 NLS-SGGSx2):
SGGSSGGSKRTADGSEFESPKKKRKVSGGSSGGS (SEQ ID NO: 105)
Genscript codon optimized MMLV RT pentamutant
(D200N T306K W313F T330P L603W);
TLNIEDEYRLHETSKEPDVSLGSTWLSDFPQAWAETGGMGLAVRQAPLIIPLKATSTPVS
IKQYPMSQEARLGIKPHIQRLLDQGILVPCQSPWNTPLLPVKKPGTNDYRPVQDLREVNK
RVEDIHPTVPNPYNLLSGLPPSHQWYTVLDLKDAFFCLRLHPTSQPLFAFEWRDPEMGIS
GQLTWTRLPQGFKNSPTLFNEALHRDLADFRIQHPDLILLQYVDDLLLAATSELDCQQGT
RALLQTLGNLGYRASAKKAQICQKQVKYLGYLLKEGQRWLTEARKETVMGQPTPKTPRQL
REFLGKAGFCRLFIPGFAEMAAPLYPLTKPGTLFNWGPDQQKAYQEIKQALLTAPALGLP
DLTKPFELFVDEKQGYAKGVLTQKLGPWRRPVAYLSKKLDPVAAGWPPCLRMVAAIAVLT
KDAGKLTMGQPLVILAPHAVEALVKQPPDRWLSNARMTHYQALLLDTDRVQFGPVVALNP
ATLLPLPEEGLQHNCLDILAEAHGTRPDLTDQPLPDADHTWYTDGSSLLQEGQRKAGAAV
TTETEVIWAKALPAGTSAQRAELIALTQALKMAEGKKLNVYTDSRYAFATAHIHGEIYRR
RGWLTSEGKEIKNKDEILALLKALFLPKRLSIIHCPGHQKGHSAEARGNRMADQAARKAA
ITETPDTSTLLIENSSP (SEQ ID NO: 98)
Other linker sequences:
SGGS (SEQ ID NO: 122)
Bipartite SV40 NLS:
KRTADGSEFESPKKKRKV (SEQ ID NO: 140)
Other linker sequences:
GSG (SEQ ID NO: 122)
c-Myc NLS:
PAAKRVKLD (SEQ ID NO: 135)

The modified fusion proteins may comprise any suitable structural configuration. For example, the fusion protein may comprise from the N-terminal to the C-terminal direction, a napDNAbp fused to a polymerase (e.g., a DNA-dependent DNA polymerase or an RNA-dependent DNA polymerase, such as a reverse transcriptase). In other embodiments, the fusion protein may comprise from the N-terminal to the C-terminal direction, a polymerase (e.g., a reverse transcriptase) fused to a napDNAbp. The fused domain may optionally be joined by a linker, e.g., an amino acid sequence. In other embodiments, the fusion proteins may comprise the structure NH₂-[napDNAbp]-[polymerase]-COOH; or NH₂-[polymerase]-[napDNAbp]-COOH, wherein each instance of “]-[” indicates the presence of an optional linker sequence. In embodiments wherein the polymerase is a reverse transcriptase, the fusion proteins may comprise the structure NH₂-[napDNAbp]-[RT]-COOH; or NH₂-[RT]-[napDNAbp]-COOH, wherein each instance of “]-[” indicates the presence of an optional linker sequence.

In various embodiments, the prime editor fusion proteins utilized in the methods and compositions contemplated herein may also include any variants of the above-disclosed sequences having an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to PEmax.

In certain embodiments, linkers may be used to link any of the peptides or peptide domains or moieties of the invention (e.g., a napDNAbp linked or fused to a reverse transcriptase).

The present disclosure contemplates the modification of any Cas9 protein known in the art with one or more of the PEmax mutations described herein (i.e., R221K, N394K, and/or H840A) and the combination of any modified Cas9 protein with one or more of the PEmax architecture features described herein (e.g., the optimized MMLV RT pentamutant, NLS's, linkers, etc.).

In some embodiments, the PEmax proteins described herein include any of the following other Cas9 sequences disclosed herein, or variants thereof, which may be further modified with one or more of the mutations described herein at corresponding amino acid positions. The napDNAbp used in the PEmax constructs described herein may include any suitable homologs and/or orthologs or naturally occurring enzymes, such as Cas9, Cas9 homologs and/or orthologs have been described in various species, including, but not limited to, S. pyogenes and S. thermophilus. The Cas moiety may be configured (e.g., mutagenized, recombinantly engineered, or otherwise obtained from nature) as a nickase, i.e., capable of cleaving only a single strand of the target double-stranded DNA. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain; that is, the Cas9 is a nickase. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs provided herein.

### Prime Editor-MMR Inhibitor Fusion Proteins

The present disclosure contemplates that in some embodiments, the MMR inhibitor (e.g., an antibody or polypeptide inhibitor of an MMR protein, such as an MLH1 dominant negative variant that inhibits MMR) may be linked to a prime editor fusion protein or at least one of the components thereof.

In certain embodiments, the inhibitor domains described herein, e.g., an anti-MLH1 antibody, or an MLH1 dominant negative variant, may also be provided in cis by fusing the domain to the prime editor domain. Any of the following structures are contemplated, wherein “]-[” denotes an optional linker:
[napDNAbp]-[reverse transcriptase]-[MLH1 inhibitor];
[reverse transcriptase]-[napDNAbp]-[MLH1 inhibitor];
[MLH1 inhibitor]-[reverse transcriptase]-[napDNAbp];
[MLH1 inhibitor]-[napDNAbp]-[reverse transcriptase];
[napDNAbp]-[MLH1 inhibitor]-[reverse transcriptase]; or
[reverse transcriptase]-[MLH1 inhibitor]-[napDNAbp].

In certain embodiments, the inhibitor domain is an MLH1 dominant negative variant. Any of the following structures are contemplated, wherein “]-[” denotes an optional linker:
[napDNAbp]-[reverse transcriptase]-[MLH1 dominant negative variant];
[reverse transcriptase]-[napDNAbp]-[MLH1 dominant negative variant];
[MLH1 dominant negative variant]-[reverse transcriptase]-[napDNAbp];
[MLH1 dominant negative variant]-[napDNAbp]-[reverse transcriptase];
[napDNAbp]-[MLH1 dominant negative variant]-[reverse transcriptase]: or
[reverse transcriptase]-[MLH1 dominant negative variant]-[napDNAbp].

In certain other embodiments, the inhibitor domains described herein, e.g., an anti-MMR protein antibody, or an MMR protein dominant negative variant, may also be provided in cis by fusing the domain to the prime editor domain. Any of the following structures are contemplated, wherein “]-[” denotes an optional linker:
[napDNAbp]-[reverse transcriptase]-[anti-MMR protein inhibitor];
[reverse transcriptase]-[napDNAbp]-[anti-MMR protein inhibitor];
[anti-MMR protein inhibitor]-[reverse transcriptase]-[napDNAbp];
[anti-MMR protein inhibitor]-[napDNAbp]-[reverse transcriptase];
[napDNAbp]-[anti-MMR protein inhibitor]-[reverse transcriptase]; or
[reverse transcriptase]-[anti-MMR protein inhibitor]-[napDNAbp], wherein the MMR protein is any one of MLH1, PMS2 (or MutL alpha), PMS1 (or MutL beta). MLH3 (or MutL gamma), MutS alpha (MSH2-MSH6), MutS beta (MSH2-MSH3). MSH2, MSH6, PCNA, RFC, EXO1, POLδ, and PCNA.

In certain embodiments, the inhibitor domain is a dominant negative variant of any MMR protein, such as MLH1, PMS2 (or MutL alpha), PMS1 (or MutL beta), MLH3 (or MutL gamma), MutS alpha (MSH2-MSH6), MutS beta (MSH2-MSH3). MSH2, MSH6, PCNA, RFC, EXO1, POLδ, and PCNA. Any of the following structures are contemplated, wherein “]-[” denotes an optional linker:
[napDNAbp]-[reverse transcriptase]-[dominant negative variant of any MMR protein];
[reverse transcriptase]-[napDNAbp]-[dominant negative variant of any MMR protein];
[dominant negative variant of any MMR protein]-[reverse transcriptase]-[napDNAbp];
[dominant negative variant of any MMR protein napDNAbp]-[reverse transcriptase];
[napDNAbp]-[dominant negative variant of any MMR protein]-[reverse transcriptase]; or [reverse transcriptase]-[dominant negative variant of any MMR protein]-[napDNAbp].

In addition, the MMR inhibitor may be fused to only one of the domains of a prime editor and administered separate from the other prime editor domain. For example, the MMR inhibitor may be fused to the napDNAbp domain, whereby the polymerase domain is provided separately in trans. In another, the MMR inhibitor may be fused to the polymerase domain, whereby the napDNAbp domain is provided separately in trans.

### A. Linkers

As defined above, the term “linker,” as used herein, refers to a chemical group or a molecule linking two molecules or moieties. e.g., a binding domain and a cleavage domain of a nuclease. In some embodiments, a linker joins a gRNA binding domain of an RNA-programmable nuclease and the catalytic domain of a polymerase (e.g., a reverse transcriptase). In some embodiments, a linker joins a dCas9 and reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid. 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)ₙ (SEQ ID NO: 118), (G)ₙ(SEQ ID NO: 119), (EAAAK)ₙ (SEQ ID NO: 120), (GGS)ₙ(SEQ ID NO: 121), (SGGS)ₙ (SEQ ID NO: 122), (XP)ₙ(SEQ ID NO: 123), or any combination thereof, wherein n is independently an integer between 1 and 30, and wherein X is any amino acid. In some embodiments, the linker comprises the amino acid sequence (GGS)n (SEQ ID NO: 121), wherein n is 1, 3, or 7. In some embodiments, the linker comprises the amino acid sequence SGSETPGTSESATPES (SEQ ID NO: 124), also referred to as XTEN. In some embodiments, the linker comprises the amino acid sequence SGGSSGGSSGSETPGTSESATPESSGGSSGGS (SEQ ID NO: 125). In some embodiments, the linker comprises the amino acid sequence SGGSGGSGGS (SEQ ID NO: 126). In some embodiments, the linker comprises the amino acid sequence SGGS (SEQ ID NO: 127). In other embodiments, the linker comprises the amino acid sequence SGGSSGGSSGSETPGTSESATPESAGSYPYDVPDYAGSAAPAAKKKKLDGSGSGGSSGGS (SEQ ID NO: 128, 60AA).

In certain embodiments, linkers may be used to link any of the peptides or peptide domains or moieties of the invention (e.g., a napDNAbp linked or fused to a reverse transcriptase).

As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a recombinase. In some embodiments, a linker joins a dCas9 and reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminoHEXAnoic acid (Ahx). In certain embodiments, the linker is based on a carbocyclic moiety (e.g., cyclopentane, cycloHEXAne). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)n (SEQ ID NO: 118), (G)n (SEQ ID NO: 119), (EAAAK)ₙ(SEQ ID NO: 120), (GGS)ₙ (SEQ ID NO: 121), (SGGS)n (SEQ ID NO: 122), (XP)n (SEQ ID NO: 123), or any combination thereof, wherein n is independently an integer between 1 and 30, and wherein X is any amino acid. In some embodiments, the linker comprises the amino acid sequence (GGS)n (SEQ ID NO: 121), wherein n is 1, 3, or 7. In some embodiments, the linker comprises the amino acid sequence SGSETPGTSESATPES (SEQ ID NO: 124), also referred to as XTEN. In some embodiments, the linker comprises the amino acid sequence SGGSSGGSSGSETPGTSESATPESSGGSSGGS (SEQ ID NO: 125). In some embodiments, the linker comprises the amino acid sequence SGGSGGSGGS (SEQ ID NO: 126). In some embodiments, the linker comprises the amino acid sequence SGGS (SEQ ID NO: 127).

In particular, the following linkers can be used in various embodiments to join prime editor domains with one another: GGS (SEQ ID NO: 129); GGSGGS (SEQ ID NO: 130); GGSGGSGGS (SEQ ID NO: 131); SGGSSGGSSGSETPGTSESATPESSGGSSGGSS (SEQ ID NO: 102); SGSETPGTSESATPES (SEQ ID NO: 124), also referred to as XTEN; SGGSSGGSSGSETPGTSESATPESAGSYPYDVPDYAGSAAPAAKKKKLDGSGSGGSSGGS (SEQ ID NO: 128).

The PE fusion proteins may also comprise various other domains besides the napDNAbp (e.g., Cas9 domain) and the polymerase domain (e.g., RT domain). For example, in the case where the napDNAbp is a Cas9 and the polymerase is a RT, the PE fusion proteins may comprise one or more linkers that join the Cas9 domain with the RT domain. The linkers may also join other functional domains, such as nuclear localization sequences (NLS) or a FEN1 (or other flap endonuclease) to the PE fusion proteins or a domain thereof.

In some embodiments, the PE fusion proteins may comprise an inhibitor of the DNA mismatch repair pathway (e.g., MLH1dn as described herein). In certain embodiments, a PE fusion protein and an inhibitor of the DNA mismatch repair pathway are fused via a linker. In some embodiments, the linker is a self-hydrolyzing linker. Suitable self-hydrolyzing linkers include, but are not limited to, amino acid sequences comprising 2A self-cleaving peptides. 2A self-cleaving peptides are capable of inducing ribosomal skipping during protein translation, resulting in the failure of the ribosome to make a peptide bond between two genes, or two gene fragments. Exemplary 2A self-cleaving peptides that may be used as linkers in the fusion proteins described herein include the amino acid sequences:

T2A-
(SEQ ID NO: 233)
EGRGSLLTCGDVEENPGP
P2A-
(SEQ ID NO: 234)
ATNFSLLKQAGDVEENPGP
E2A-
(SEQ ID NO: 235)
QCTNYALLKLAGDVESNPGP
F2A-
(SEQ ID NO: 236)
VKQTLNFDLLKLAGDVESNPGP

In certain embodiments, the PE fusion proteins described herein are fused to MLH1dn by a linker comprising the amino acid sequence of SEQ ID NO: 234.

### B. Nuclear Localization Sequence (NLS)

In various embodiments, the PE fusion proteins may comprise one or more nuclear localization sequences (NLS), which help promote translocation of a protein into the cell nucleus. Such sequences are well-known in the art and can include the examples represented by SEQ ID NOs: 1, 101, 103, 133-139.

The NLS examples above are non-limiting. The PE fusion proteins may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

In various embodiments, the prime editors and constructs encoding the prime editors utilized in the methods and compositions disclosed herein further comprise one or more, preferably, at least two nuclear localization signals. In certain embodiments, the prime editors comprise at least two NLSs. In embodiments with at least two NLSs, the NLSs can be the same NLSs or they can be different NLSs. In addition, the NLSs may be expressed as part of a fusion protein with the remaining portions of the prime editors. In some embodiments, one or more of the NLSs are bipartite NLSs (“bpNLS”). In certain embodiments, the disclosed fusion proteins comprise two bipartite NLSs. In some embodiments, the disclosed fusion proteins comprise more than two bipartite NLSs.

The location of the NLS fusion can be at the N-terminus, the C-terminus, or within a sequence of a prime editor (e.g., inserted between the encoded napDNAbp component (e.g., Cas9) and a polymerase domain (e.g., a reverse transcriptase domain).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations). The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 132), MDSLLMNRRKFLYQFKNVRWAKGRRETYLC (SEQ ID NO: 1), KRTADGSEFESPKKKRKV (SEQ ID NO: 140), or KRTADGSEFEPKKKRKV (SEQ ID NO: 141). In other embodiments. NLS comprises the amino acid sequences NLSKRPAAIKKAGQAKKKK (SEQ ID NO: 142), PAAKRVKLD (SEQ ID NO: 135), RQRRNELKRSF (SEQ ID NO: 143), NQSSNFGPMKGGNFGGRSSGPYGGGGQYFAKPRNQGGY (SEQ ID NO: 144).

In one aspect of the disclosure, a prime editor may be modified with one or more nuclear localization signals (NLS), preferably at least two NLSs. In certain embodiments, the prime editors are modified with two or more NLSs. The disclosure contemplates the use of any nuclear localization signal known in the art at the time of the disclosure, or any nuclear localization signal that is identified or otherwise made available in the state of the art after the time of the instant filing. A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 132)); (ii) a bipartite motif consisting of two basic domains separated by a variable number of spacer amino acids and exemplified by the Xenopus nucleoplasmin NLS (KRXXXXXXXXXXKKKK (SEQ ID NO: 145)); and (iii) noncanonical sequences such as M9 of the hnRNP A1 protein, the influenza virus nucleoprotein NLS, and the yeast Gal4 protein NLS (Dingwall and Laskey 1991). Nuclear localization signals appear at various points in the amino acid sequences of proteins. NLS's have been identified at the N-terminus, the C-terminus and in the central region of proteins. Thus, the disclosure provides prime editors that may be modified with one or more NLSs at the C-terminus, the N-terminus, as well as at in internal region of the prime editor. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a prime editor to include one or more NLSs. In one aspect, the prime editors may be engineered to express a prime editor protein that is translationally fused at its N-terminus or its C-terminus (or both) to one or more NLSs, i.e., to form a prime editor-NLS fusion construct. In other embodiments, the prime editor-encoding nucleotide sequence may be genetically modified to incorporate a reading frame that encodes one or more NLSs in an internal region of the encoded prime editor. In addition, the NLSs may include various amino acid linkers or spacer regions encoded between the prime editor and the N-terminally, C-terminally, or internally-attached NLS amino acid sequence, e.g., and in the central region of proteins. Thus, the present disclosure also provides for nucleotide constructs, vectors, and host cells for expressing fusion proteins that comprise a prime editor and one or more NLSs.

The prime editors utilized in the methods and compositions described herein may also comprise nuclear localization signals which are linked to a prime editor through one or more linkers, e.g., and polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and be joined to the prime editor by any suitable strategy that effectuates forming a bond (e.g., covalent linkage, hydrogen bonding) between the prime editor and the one or more NLSs.

### C. Flap Endonucleases (e.g., FEN1)

In various embodiments, the PE fusion proteins may comprise one or more flap endonucleases (e.g., FEN1), which refers to an enzyme that catalyzes the removal of 5′ single strand DNA flaps. These are enzymes that process the removal of 5′ flaps formed during cellular processes, including DNA replication. The prime editing utilized in the methods and compositions described herein may utilize endogenously supplied flap endonucleases or those provided in trans to remove the 5′ flap of endogenous DNA formed at the target site during prime editing. Flap endonucleases are known in the art and can be found described in Patel et al., “Flap endonucleases pass 5′-flaps through a flexible arch using a disorder-thread-order mechanism to confer specificity for free 5′-ends,” Nucleic Acids Research, 2012, 40(10): 4507-4519 and Tsutakawa et al., “Human flap endonuclease structures, DNA double-base flipping, and a unified understanding of the FEN1 superfamily,” Cell. 2011, 145(2): 198-211 (each of which are incorporated herein by reference). An exemplary flap endonuclease is FEN1, which can be represented by the following amino acid sequence:

| | | SEQ |
|---|---|---|
| Description | Sequence | ID NO: |
| FEN1 | MGIQGLAKLIADVAPSAIRENDIKSY | 146 |
| Wild type | FGRKVAIDASMSIYQFLIAVRQGGDV | |
| (wt) | LQNEEGETTSHLMGMFYRTIRMMENG | |
| | IKPVYVFDGKPPQLKSGELAKRSERR | |
| | AEAEKQLQQAQAAGAEQEVEKFTKRL | |
| | VKVTKQHNDECKHLLSLMGIPYLDAP | |
| | SEAEASCAALVKAGKVYAAATEDMDC | |
| | LTFGSPVLMRHLTASEAKKLPIQEFH | |
| | LSRILQELGLNQEQFVDLCILLGSDY | |
| | CESIRGIGPKRAVDLIQKHKSIEEIV | |
| | RRLDPNKYPVPENWLHKEAHQLFLEP | |
| | EVLDPESVELKWSEPNEEELIKFMCG | |
| | EKQFSEERIRSGVKRLSKSRQGSTQG | |
| | RLDDFFKVTGSLSSAKRKEPEPKGST | |
| | KKKAKTGAAGKFKRGK | |

The flap endonucleases may also include any FEN 1 variant, mutant, or other flap endonuclease ortholog, homolog, or variant. Non-limiting FEN 1 variant examples are as follows:

| | | SEQ |
|---|---|---|
| Description | Sequence | ID NO: |
| FEN1 | MGIQGLAKLIADVAPSAIRENDIKSYFGRKVAIDASMSIYQFLIAVRQGGDVLQ | 147 |
| K168R (relative | NEEGETTSHLMGMFYRTIRMMENGIKPVYVFDGKPPQLKSGELAKRSERRAEAE | |
| to FEN1 wt) | KQLQQAQAAGAEQEVEKFTKRLVKVTKQHNDECKHLLSLMGIPYLDAPSEAEAS | |
| | CAALVRAGKVYAAATEDMDCLTFGSPVLMRHLTASEAKKLPIQEFHLSRILQEL | |
| | GLNQEQFVDLCILLGSDYCESIRGIGPKRAVDLIQKHKSIEEIVRRLDPNKYPV | |
| | PENWLHKEAHQLFLEPEVLDPESVELKWSEPNEEELIKFMCGEKQFSEERIRSG | |
| | VKRLSKSRQGSTQGRLDDFFKVTGSLSSAKRKEPEPKGSTKKKAKTGAAGKFKR | |
| | GK | |
| FEN1 | MGIQGLAKLIADVAPSAIRENDIKSYFGRKVAIDASMSIYQFLIAVRQGGDVLQ | 148 |
| S187A (relative | NEEGETTSHLMGMFYRTIRMMENGIKPVYVEDGKPPQLKSGELAKRSERRAEAE | |
| to FENI wt) | KQLQQAQAAGAEQEVEKFTKRLVKVTKQHNDECKHLLSLMGIPYLDAPSEAEAS | |
| | CAALVKAGKVYAAATEDMDCLTFGAPVLMRHLTASEAKKLPIQEFHLSRILQEL | |
| | GLNQEQFVDLCILLGSDYCESIRGIGPKRAVDLIQKHKSIEEIVRRLDPNKYPV | |
| | PENWLHKEAHQLFLEPEVLDPESVELKWSEPNEEELIKFMCGEKQFSEERIRSG | |
| | VKRLSKSRQGSTQGRLDDFFKVTGSLSSAKRKEPEPKGSTKKKAKTGAAGKFKR | |
| | GK | |
| FEN1 | MGIQGLAKLIADVAPSAIRENDIKSYFGRKVAIDASMSIYQFLIAVRQGGDVLQ | 149 |
| K354R (relative | NEEGETTSHLMGMFYRTIRMMENGIKPVYVFDGKPPQLKSGELAKRSERRAEAE | |
| to FEN1 wt) | KQLQQAQAAGAEQEVEKFTKRLVKVTKQHNDECKHLLSLMGIPYLDAPSEAEAS | |
| | CAALVKAGKVYAAATEDMDCLTFGSPVLMRHLTASEAKKLPIQEFHLSRILQEL | |
| | GLNQEQFVDLCILLGSDYCESIRGIGPKRAVDLIQKHKSIEEIVRRLDPNKYPV | |
| | PENWLHKEAHQLFLEPEVLDPESVELKWSEPNEEELIKFMCGEKQFSEERIRSG | |
| | VKRLSKSRQGSTQGRLDDFFKVTGSLSSARRKEPEPKGSTKKKAKTGAAGKFKR | |
| | GK | |
| GEN1 | MGVNDLWQILEPVKQHIPLRNLGGKTIAVDLSLWVCEAQTVKKMMGSVMKPHLR | 150 |
| | NLFFRISYLTQMDVKLVFVMEGEPPKLKADVISKRNQSRYGSSGKSWSQKTGRS | |
| | HFKSVLRECLHMLECLGIPWVQAAGEAEAMCAYLNAGGHVDGCLTNDGDTFLYG | |
| | AQTVYRNFTMNTKDPHVDCYTMSSIKSKLGLDRDALVGLAILLGCDYLPKGVPG | |
| | VGKEQALKLIQILKGQSLLQRFNRWNETSCNSSPQLLVTKKLAHCSVCSHPGSP | |
| | KDHERNGCRLCKSDKYCEPHDYEYCCPCEWHRTEHDRQLSEVENNIKKKACCCE | |
| | GFPFHEVIQEFLLNKDKLVKVIRYQRPDLLLFQRFTLEKMEWPNHYACEKLLVL | |
| | LTHYDMIERKLGSRNSNQLQPIRIVKTRIRNGVHCFEIEWEKPEHYAMEDKQHG | |
| | EFALLTIEEESLFEAAYPEIVAVYQKQKLEIKGKKQKRIKPKENNLPEPDEVMS | |
| | FQSHMTLKPTCEIFHKQNSKLNSGISPDPTLPQESISASLNSLLLPKNTPCLNA | |
| | QEQFMSSLRPLAIQQIKAVSKSLISESSQPNTSSHNISVIADLHLSTIDWEGTS | |
| | FSNSPAIQRNTFSHDLKSEVESELSAIPDGFENIPEQLSCESERYTANIKKVLD | |
| | EDSDGISPEEHLLSGITDLCLQDLPLKERIFTKLSYPQDNLQPDVNLKTLSILS | |
| | VKESCIANSGSDCTSHLSKDLPGIPLQNESRDSKILKGDQLLQEDYKVNTSVPY | |
| | SVSNTVVKTCNVRPPNTALDHSRKVDMQTTRKILMKKSVCLDRHSSDEQSAPVF | |
| | GKAKYTTQRMKHSSQKHNSSHFKESGHNKLSSPKIHIKETEQCVRSYETAENEE | |
| | SCFPDSTKSSLSSLQCHKKENNSGTCLDSPLPLRQRLKLRFQST | |
| ERCC5 | MGVQGLWKLLECSGRQVSPEALEGKILAVDISIWLNQALKGVRDRHGNSIENPH | 151 |
| | LLTLFHRLCKLLFFRIRPIFVFDGDAPLLKKQTLVKRRQRKDLASSDSRKTTEK | |
| | LLKTFLKRQAIKTAFRSKRDEALPSLTQVRRENDLYVLPPLQEEEKHSSEEEDE | |
| | KEWQERMNQKQALQEEFFHNPQAIDIESEDFSSLPPEVKHEILTDMKEFTKRRR | |
| | TLFEAMPEESDDFSQYQLKGLLKKNYLNQHIEHVQKEMNQQHSGHIRRQYEDEG | |
| | GFLKEVESRRVVSEDTSHYILIKGIQAKTVAEVDSESLPSSSKMHGMSFDVKSS | |
| | PCEKLKTEKEPDATPPSPRTLLAMQAALLGSSSEEELESENRRQARGRNAPAAV | |
| | DEGSISPRTLSAIKRALDDDEDVKVCAGDDVQTGGPGAEEMRINSSTENSDEGL | |
| | KVRDGKGIPFTATLASSSVNSAEEHVASTNEGREPTDSVPKEQMSLVHVGTEAF | |
| | PISDESMIKDRKDRLPLESAVVRHSDAPGLPNGRELTPASPTCTNSVSKNETHA | |
| | EVLEQQNELCPYESKFDSSLLSSDDETKCKPNSASEVIGPVSLQETSSIVSVPS | |
| | EAVDNVENVVSFNAKEHENFLETIQEQQTTESAGQDLISIPKAVEPMEIDSEES | |
| | ESDGSFIEVQSVISDEELQAEFPETSKPPSEQGEEELVGTREGEAPAESESLLR | |
| | DNSERDDVDGEPQEAEKDAEDSLHEWQDINLEELETLESNLLAQQNSLKAQKQQ | |
| | QERIAATVTGQMFLESQELLRLFGIPYIQAPMEAEAQCAILDLTDQTSGTITDD | |
| | SDIWLFGARHVYRNFFNKNKFVEYYQYVDFHNQLGLDRNKLINLAYLLGSDYTE | |
| | GIPTVGCVTAMEILNEFPGHGLEPLLKFSEWWHEAQKNPKIRPNPHDTKVKKKL | |
| | RTLQLTPGFPNPAVAEAYLKPVVDDSKGSFLWGKPDLDKIREFCQRYFGWNRTK | |
| | TDESLFPVLKQLDAQQTQLRIDSFFRLAQQEKEDAKRIKSQRLNRAVTCMLRKE | |
| | KEAAASEIEAVSVAMEKEFELLDKAKRKTQKRGITNTLEESSSLKRKRLSDSKR | |
| | KNTCGGFLGETCLSESSDGSSSEDAESSSLMNVQRRTAAKEPKTSASDSQNSVK | |
| | EAPVKNGGATTSSSSDSDDDGGKEKMVLVTARSVFGKKRRKLRRARGRKRKT | |

In various embodiments, the prime editor fusion proteins utilized in the methods and compositions contemplated herein may include any flap endonuclease variant of the above-disclosed sequences having an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the above sequences. Other endonucleases that may be utilized by the instant methods to facilitate removal of the 5′ end single strand DNA flap include, but are not limited to (1) trex 2, (2) exo1 endonuclease (e.g., Keijzers et al., Biosci Rep. 2015, 35(3): e00206)

### Trex 2

3′ three prime repair exonuclease 2 (TREX2)-
human [Accession No. NM_080701]
(SEQ ID NO: 152)
MSEAPRAETFVFLDLEATGLPSVEPEIAELSLFAVHRSSLENPEH
DESGALVLPRVLDKLTLCMCPERPFTAKASEITGLSSEGLARCRK
AGFDGAVVRTLQAFLSRQAGPICLVAHNGEDYDFPLLCAELRRLG
ARLPRDTVCLDTLPALRGLDRAHSHGTRARGRQGYSLGSLFHRYF
RAEPSAAHSAEGDVHTLLLIFLHRAAELLAWADEQARGWAHIEPM
YLPPDDPSLEA.
3′ three prime repair exonuclease 2 (TREX2)-
mouse [Accession No. NM_011907]
(SEQ ID NO: 153)
MSEPPRAETFVFLDLEATGLPNMDPEIAEISLFAVHRSSLENPER
DDSGSLVLPRVLDKLTLCMCPERPFTAKASEITGLSSESLMHCGK
AGENGAVVRTLQGFLSRQEGPICLVAHNGFDYDFPLLCTELQRLG
AHLPQDTVCLDTLPALRGLDRAHSHGTRAQGRKSYSLASLFHRYF
QAEPSAAHSAEGDVHTLLLIFLHRAPELLAWADEQARSWAHIEPM
YVPPDGPSLEA.
3′ three prime repair exonuclease 2 (TREX2)-rat
[Accession No. NM_001107580]
(SEQ ID NO: 154)
MSEPLRAETFVFLDLEATGLPNMDPEIAEISLFAVHRSSLENPER
DDSGSLVLPRVLDKLTLCMCPERPFTAKASEITGLSSEGLMNCRK
AAFNDAVVRTLQGFLSRQEGPICLVAHNGFDYDFPLLCTELQRLG
AHLPRDTVCLDTLPALRGLDRVHSHGTRAQGRKSYSLASLFHRYF
QAEPSAAHSAEGDVNTLLLIFLHRAPELLAWADEQARSWAHIEPM
YVPPDGPSLEA.

### ExoI

Human exonuclease 1 (EXO1) has been implicated in many different DNA metabolic processes, including DNA mismatch repair (MMR), micro-mediated end-joining, homologous recombination (HR), and replication. Human EXO1 belongs to a family of eukaryotic nucleases. Rad2/XPG, which also include FEN1 and GEN1. The Rad2/XPG family is conserved in the nuclease domain through species from phage to human. The EXO1 gene product exhibits both 5′ exonuclease and 5′ flap activity. Additionally, EXO1 contains an intrinsic 5′ RNase H activity. Human EXO1 has a high affinity for processing double stranded DNA (dsDNA), nicks, gaps, pseudo Y structures and can resolve Holliday junctions using its inherent flap activity. Human EXO1 is implicated in MMR and contain conserved binding domains interacting directly with MLH1 and MSH2. EXO1 nucleolytic activity is positively stimulated by PCNA, MutSα (MSH2/MSH6 complex), 14-3-3, MRN and 9-1-1 complex.

exonuclease 1 (EXO1) Accession No. NM_003686
(Homo sapiens exonuclease 1
(EXO1), transcript variant 3)-isoform A
(SEQ ID NO: 155)
MGIQGLLQFIKEASEPIHVRKYKGQVVAVDTYCWLHKGAIACAEK
LAKGEPTDRYVGFCMKFVNMLLSHGIKPILVFDGCTLPSKKEVER
SRRERRQANLLKGKQLLREGKVSEARECFTRSINITHAMAHKVIK
AARSQGVDCLVAPYEADAQLAYLNKAGIVQAIITEDSDLLAFGCK
KVILKMDQFGNGLEIDQARLGMCRQLGDVFTEEKFRYMCILSGCD
YLSSLRGIGLAKACKVLRLANNPDIVKVIKKIGHYLKMNITVPED
YINGFIRANNTFLYQLVFDPIKRKLIPLNAYEDDVDPETLSYAGQ
YVDDSIALQIALGNKDINTFEQIDDYNPDTAMPAHSRSHSWDDKT
CQKSANVSSIWHRNYSPRPESGTVSDAPQLKENPSTVGVERVIST
KGLNLPRKSSIVKRPRSAELSEDDLLSQYSLSFTKKTKKNSSEGN
KSLSFSEVFVPDLVNGPINKKSVSTPPRTRNKFATFLQRKNEESG
AVVVPGTRSRFFCSSDSTDCVSNKVSIQPLDETAVTDKENNLHES
EYGDQEGKRLVDTDVARNSSDDIPNNHIPGDHIPDKATVFTDEES
YSFESSKFTRTISPPTLGTLRSCFSWSGGLGDFSRTPSPSPSTAL
QQFRRKSDSPTSLPENNMSDVSQLKSEESSDDESHPLREEACSSQ
SQESGEFSLQSSNASKLSQCSSKDSDSEESDCNIKLLDSQSDQTS
KLRLSHFSKKDTPLRNKVPGLYKSSSADSLSTTKIKPLGPARASG
LSKKPASIQKRKHHNAENKPGLQIKLNELWKNFGFKKF.
exonuclease 1 (EXO1) Accession No. NM_006027
(Homo sapiens exonuclease 1
(EXO1), transcript variant 3)-isoform B
(SEQ ID NO: 156)
MGIQGLLQFIKEASEPIHVRKYKGQVVAVDTYCWLHKGAIACAEK
LAKGEPTDRYVGFCMKFVNMLLSHGIKPILVFDGCTLPSKKEVER
SRRERRQANLLKGKQLLREGKVSEARECFTRSINITHAMAHKVIK
AARSQGVDCLVAPYEADAQLAYLNKAGIVQAIITEDSDLLAFGCK
KVILKMDQFGNGLEIDQARLGMCRQLGDVFTEEKFRYMCILSGCD
YLSSLRGIGLAKACKVLRLANNPDIVKVIKKIGHYLKMNITVPED
YINGFIRANNTFLYQLVFDPIKRKLIPLNAYEDDVDPETLSYAGQ
YVDDSIALQIALGNKDINTFEQIDDYNPDTAMPAHSRSHSWDDKT
CQKSANVSSIWHRNYSPRPESGTVSDAPQLKENPSTVGVERVIST
KGLNLPRKSSIVKRPRSAELSEDDLLSQYSLSFTKKTKKNSSEGN
KSLSFSEVFVPDLVNGPTNKKSVSTPPRTRNKFATFLQRKNEESG
AVVVPGTRSRFFCSSDSTDCVSNKVSIQPLDETAVTDKENNLHES
EYGDQEGKRLVDTDVARNSSDDIPNNHIPGDHIPDKATVFTDEES
YSFESSKFTRTISPPTLGTLRSCFSWSGGLGDFSRTPSPSPSTAL
QQFRRKSDSPTSLPENNMSDVSQLKSEESSDDESHPLREEACSSQ
SQESGEFSLQSSNASKLSQCSSKDSDSEESDCNIKLLDSQSDQTS
KLRLSHFSKKDTPLRNKVPGLYKSSSADSLSTTKIKPLGPARASG
LSKKPASIQKRKHHNAENKPGLQIKLNELWKNFGFKKDSEKLPPC
KKPLSPVRDNIQLTPEAEEDIFNKPECGRVQRAIFQ.
exonuclease 1 (EXO1) Accession No. NM_001319224
(Homo sapiens exonuclease 1
(EXO1), transcript variant 4)-isoform C
(SEQ ID NO: 157)
MGIQGLLQFIKEASEPIHVRKYKGQVVAVDTYCWLHKGAIACAEK
LAKGEPTDRYVGFCMKFVNMLLSHGIKPILVFDGCTLPSKKEVER
SRRERRQANLLKGKQLLREGKVSEARECFTRSINITHAMAHKVIK
AARSQGVDCLVAPYEADAQLAYLNKAGIVQAITEDSDLLAFGCKK
VILKMDQFGNGLEIDQARLGMCRQLGDVFTEEKFRYMCILSGCDY
LSSLRGIGLAKACKVLRLANNPDIVKVIKKIGHYLKMNITVPEDY
INGFIRANNTFLYQLVFDPIKRKLIPLNAYEDDVDPETLSYAGQY
VDDSIALQIALGNKDINTFEQIDDYNPDTAMPAHSRSHSWDDKTC
QKSANVSSIWHRNYSPRPESGTVSDAPQLKENPSTVGVERVISTK
GLNLPRKSSIVKRPRSELSEDDLLSQYSLSFTKKTKKNSSEGNKS
LSFSEVFVPDLVNGPINKKSVSTPPRTRNKFATFLQRKNEESGAV
VVPGTRSRFFCSSDSTDCVSNKVSIQPLDETAVTDKENNLHESEY
GDQEGKRLVDTDVARNSSDDIPNNHIPGDHIPDKATVFTDEESYS
FESSKFTRTISPPTLGTLRSCFSWSGGLGDFSRTPSPSPSTALQQ
FRRKSDSPTSLPENNMSDVSQLKSEESSDDESHPLREEACSSQSQ
ESGEFSLQSSNASKLSQCSSKDSDSEESDCNIKLLDSQSDQTSKL
RLSHFSKKDTPLRNKVPGLYKSSSADSLSTTKIKPLGPARASGLS
KKPASIQKRKHHNAENKPGLQIKLNELWKNFGFKKDSEKLPPCKK
PLSPVRDNIQLTPEAEEDIFNKPECGRVQRAIFQ.

### D. Inteins and Split-Inteins

It will be understood that in some embodiments (e.g., delivery of a prime editor in vivo using AAV particles), it may be advantageous to split a polypeptide (e.g., a deaminase or a napDNAbp) or a fusion protein (e.g., a prime editor) into an N-terminal half and a C-terminal half, delivery them separately, and then allow their colocalization to reform the complete protein (or fusion protein as the case may be) within the cell. Separate halves of a protein or a fusion protein may each comprise a split-intein tag to facilitate the reformation of the complete protein or fusion protein by the mechanism of protein trans splicing.

Protein trans-splicing, catalyzed by split inteins, provides an entirely enzymatic method for protein ligation. A split-intein is essentially a contiguous intein (e.g., a mini-intein) split into two pieces named N-intein and C-intein, respectively. The N-intein and C-intein of a split intein can associate non-covalently to form an active intein and catalyze the splicing reaction essentially in same way as a contiguous intein does. Split inteins have been found in nature and also engineered in laboratories. As used herein, the term “split intein” refers to any intein in which one or more peptide bond breaks exists between the N-terminal and C-terminal amino acid sequences such that the N-terminal and C-terminal sequences become separate molecules that can non-covalently reassociate, or reconstitute, into an intein that is functional for trans-splicing reactions. Any catalytically active intein, or fragment thereof, may be used to derive a split intein for use in the methods of the invention. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing trans-splicing reactions.

As used herein, the “N-terminal split intein (In)” refers to any intein sequence that comprises an N-terminal amino acid sequence that is functional for trans-splicing reactions. An In thus also comprises a sequence that is spliced out when trans-splicing occurs. An In can comprise a sequence that is a modification of the N-terminal portion of a naturally occurring intein sequence. For example, an In can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the In.

As used herein, the “C-terminal split intein (Ic)” refers to any intein sequence that comprises a C-terminal amino acid sequence that is functional for trans-splicing reactions. In one aspect, the Ic comprises 4 to 7 contiguous amino acid residues, at least 4 amino acids of which are from the last β-strand of the intein from which it was derived. An Ic thus also comprises a sequence that is spliced out when trans-splicing occurs. An Ic can comprise a sequence that is a modification of the C-terminal portion of a naturally occurring intein sequence. For example, an Ic can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the Ic.

In some embodiments of the invention, a peptide linked to an Ic or an In can comprise an additional chemical moiety including, among others, fluorescence groups, biotin, polyethylene glycol (PEG), amino acid analogs, unnatural amino acids, phosphate groups, glycosyl groups, radioisotope labels, and pharmaceutical molecules. In other embodiments, a peptide linked to an Ic can comprise one or more chemically reactive groups including, among others, ketone, aldehyde. Cys residues and Lys residues. The N-intein and C-intein of a split intein can associate non-covalently to form an active intein and catalyze the splicing reaction when an “intein-splicing polypeptide (ISP)” is present. As used herein, “intein-splicing polypeptide (ISP)” refers to the portion of the amino acid sequence of a split intein that remains when the Ic, In, or both, are removed from the split intein. In certain embodiments, the In comprises the ISP. In another embodiment, the Ic comprises the ISP. In yet another embodiment, the ISP is a separate peptide that is not covalently linked to In nor to Ic.

Split inteins may be created from contiguous inteins by engineering one or more split sites in the unstructured loop or intervening amino acid sequence between the −12 conserved beta-strands found in the structure of mini-inteins. Some flexibility in the position of the split site within regions between the beta-strands may exist, provided that creation of the split will not disrupt the structure of the intein, the structured beta-strands in particular, to a sufficient degree that protein splicing activity is lost.

In protein trans-splicing, one precursor protein consists of an N-extein part followed by the N-intein, another precursor protein consists of the C-intein followed by a C-extein part, and a trans-splicing reaction (catalyzed by the N- and C-inteins together) excises the two intein sequences and links the two extein sequences with a peptide bond. Protein trans-splicing, being an enzymatic reaction, can work with very low (e.g., micromolar) concentrations of proteins and can be carried out under physiological conditions.

Exemplary sequences are as follows:

| NAME | SEQUENCE OF LIGAND-DEPENDENT INTEIN |
|---|---|
| 2-4 | CLAEGTRIFDPVTGTTHRIEDVVDGRKPIHVVAAAKDGTL |
| INTEIN: | LARPVVSWFDQGTRDVIGLRIAGGAIVWATPDHKVLTEYG |
| | WRAAGELRKGDRVAGPGGSGNSLALSLTADQMVSALLDAE |
| | PPILYSEYDPTSPFSEASMMGLLTNLADRELVHMINWAKR |
| | VPGFVDLTLHDQAHLLECAWLEILMIGLVWRSMEHPGKLL |
| | FAPNLLLDRNQGKCVEGMVEIFDMLLATSSRFRMMNLQGE |
| | EFVCLKSIILLNSGVYTFLSSTLKSLEEKDHIHRALDKIT |
| | DTLIHLMAKAGLTLQQQHQRLAQLLLILSHIRHMSNKGME |
| | HLYSMKYKNVVPLYDLLLEMLDAHRLHAGGSGASRVQAFA |
| | DALDDKFLHDMLAEELRYSVIREVLPTRRARTFDLEVEEL |
| | HTLVAEGVVVHNC (SEQ ID NO: 158) |
| 3-2 | CLAEGTRIFDPVTGTTHRIEDVVDGRKPIHVVAVAKDGTL |
| INTEIN | LARPVVSWFDQGTRDVIGLRIAGGAIVWATPDHKVLTEYG |
| | WRAAGELRKGDRVAGPGGSGNSLALSLTADQMVSALLDAE |
| | PPILYSEYDPTSPFSEASMMGLLTNLADRELVHMINWAKR |
| | VPGFVDLTLHDQAHLLECAWLEILMIGLVWRSMEHPGKLL |
| | FAPNLLLDRNQGKCVEGMVEIFDMLLATSSRFRMMNLQGE |
| | EFVCLKSIILLNSGVYTFLSSTLKSLEEKDHIHRALDKIT |
| | DTLIHLMAKAGLTLQQQHQRLAQLLLILSHIRHMSNKGME |
| | HLYSMKYTNVVPLYDLLLEMLDAHRLHAGGSGASRVQAFA |
| | DALDDKFLHDMLAEELRYSVIREVLPTRRARTFDLEVEEL |
| | HTLVAEGVVVHNC (SEQ ID NO: 159) |
| 30R3-1 | CLAEGTRIFDPVTGTTHRIEDVVDGRKPIHVVAAAKDGTL |
| INTEIN | LARPVVSWFDQGTRDVIGLRIAGGATVWATPDHKVLTEYG |
| | WRAAGELRKGDRVAGPGGSGNSLALSLTADQMVSALLDAE |
| | PPIPYSEYDPTSPFSEASMMGLLTNLADRELVHMINWAKR |
| | VPGFVDLTLHDQAHLLECAWLEILMIGLVWRSMEHPGKLL |
| | FAPNLLLDRNQGKCVEGMVEIFDMLLATSSRFRMMNLQGE |
| | EFVCLKSIILLNSGVYTFLSSTLKSLEEKDHIHRALDKIT |
| | DTLIHLMAKAGLTLQQQHQRLAQLLLILSHIRHMSNKGME |
| | HLYSMKYKNVVPLYDLLLEMLDAHRLHAGGSGASRVQAFA |
| | DALDDKFLHDMLAEGLRYSVIREVLPTRRARTFDLEVEEL |
| | HTLVAEGVVVHNC (SEQ ID NO: 160) |
| 30R3-2 | CLAEGTRIFDPVTGTTHRIEDVVDGRKPIHVVAAAKDGTL |
| INTEIN | LARPVVSWFDQGTRDVIGLRIAGGATVWATPDHKVLTEYG |
| | WRAAGELRKGDRVAGPGGSGNSLALSLTADQMVSALLDAE |
| | PPILYSEYDPTSPFSEASMMGLLTNLADRELVHMINWAKR |
| | VPGFVDLTLHDQAHLLECAWLEILMIGLVWRSMEHPGKLL |
| | FAPNLLLDRNQGKCVEGMVEIFDMLLATSSRFRMMNLQGE |
| | EFVCLKSIILLNSGVYTFLSSTLKSLEEKDHIHRALDKIT |
| | DTLIHLMAKAGLTLQQQHQRLAQLLLILSHIRHMSNKGME |
| | HLYSMKYKNVVPLYDLLLEMLDAHRLHAGGSGASRVQAFA |
| | DALDDKFLHDMLAEELRYSVIREVLPTRRARTFDLEVEEL |
| | HTLVAEGVVVHNC (SEQ ID NO: 161) |
| 30R3-3 | CLAEGTRIFDPVTGTTHRIEDVVDGRKPIHVVAAAKDGTL |
| INTEIN | LARPVVSWFDQGTRDVIGLRIAGGATVWATPDHKVLTEYG |
| | WRAAGELRKGDRVAGPGGSGNSLALSLTADQMVSALLDAE |
| | PPIPYSEYDPTSPFSEASMMGLLTNLADRELVHMINWAKR |
| | VPGFVDLTLHDQAHLLECAWLEILMIGLVWRSMEHPGKLL |
| | FAPNLLLDRNQGKCVEGMVEIFDMLLATSSRFRMMNLQGE |
| | EFVCLKSIILLNSGVYTFLSSTLKSLEEKDHIHRALDKIT |
| | DTLIHLMAKAGLTLQQQHQRLAQLLLILSHIRHMSNKGME |
| | HLYSMKYKNVVPLYDLLLEMLDAHRLHAGGSGASRVQAFA |
| | DALDDKFLHDMLAEELRYSVIREVLPTRRARTFDLEVEEL |
| | HTLVAEGVVVHNC (SEQ ID NO: 162) |
| 37R3-1 | CLAEGTRIFDPVTGTTHRIEDVVDGRKPIHVVAAAKDGTL |
| INTEIN | LARPVVSWFDQGTRDVIGLRIAGGATVWATPDHKVLTEYG |
| | WRAAGELRKGDRVAGPGGSGNSLALSLTADQMVSALLDAE |
| | PPILYSEYNPTSPFSEASMMGLLTNLADRELVHMINWAKR |
| | VPGFVDLTLHDQAHLLERAWLEILMIGLVWRSMEHPGKLL |
| | FAPNLLLDRNQGKCVEGMVEIFDMLLATSSRFRMMNLQGE |
| | EFVCLKSIILLNSGVYTFLSSTLKSLEEKDHIHRALDKIT |
| | DTLIHLMAKAGLTLQQQHQRLAQLLLILSHIRHMSNKGME |
| | HLYSMKYKNVVPLYDLLLEMLDAHRLHAGGSGASRVQAFA |
| | DALDDKFLHDMLAEGLRYSVIREVLPTRRARTFDLEVEEL |
| | HTLVAEGVVVHNC |
| | ((SEQ ID NO: 163) |
| 37R3-2 | CLAEGTRIFDPVTGTTHRIEDVVDGRKPIHVVAAAKDGTL |
| INTEIN | LARPVVSWFDQGTRDVIGLRIAGGAIVWATPDHKVLTEYG |
| | WRAAGELRKGDRVAGPGGSGNSLALSLTADQMVSALLDAE |
| | PPILYSEYDPTSPFSEASMMGLLTNLADRELVHMINWAKR |
| | VPGFVDLTLHDQAHLLERAWLEILMIGLVWRSMEHPGKLL |
| | FAPNLLLDRNQGKCVEGMVEIFDMLLATSSRFRMMNLQGE |
| | EFVCLKSIILLNSGVYTFLSSTLKSLEEKDHIHRALDKIT |
| | DTLIHLMAKAGLTLQQQHQRLAQLLLILSHIRHMSNKGME |
| | HLYSMKYKNVVPLYDLLLEMLDAHRLHAGGSGASRVQAFA |
| | DALDDKFLHDMLAEGLRYSVIREVLPTRRARTFDLEVEEL |
| | HTLVAEGVVVHNC (SEQ ID NO: 164) |
| 37R3-3 | CLAEGTRIFDPVTGTTHRIEDVVDGRKPIHVVAVAKDGTL |
| INTEIN | LARPVVSWFDQGTRDVIGLRIAGGATVWATPDHKVLTEYG |
| | WRAAGELRKGDRVAGPGGSGNSLALSLTADQMVSALLDAE |
| | PPILYSEYDPTSPFSEASMMGLLTNLADRELVHMINWAKR |
| | VPGFVDLTLHDQAHLLERAWLEILMIGLVWRSMEHPGKLL |
| | FAPNLLLDRNQGKCVEGMVEIFDMLLATSSRFRMMNLQGE |
| | EFVCLKSIILLNSGVYTFLSSTLKSLEEKDHIHRALDKIT |
| | DTLIHLMAKAGLTLQQQHQRLAQLLLILSHIRHMSNKGME |
| | HLYSMKYKNVVPLYDLLLEMLDAHRLHAGGSGASRVQAFA |
| | DALDDKFLHDMLAEELRYSVIREVLPTRRARTFDLEVEEL |
| | HTLVAEGVVVHNC (SEQ ID NO: 165) |

Although inteins are most frequently found as a contiguous domain, some exist in a naturally split form. In this case, the two fragments are expressed as separate polypeptides and must associate before splicing takes place, so-called protein trans-splicing.

An exemplary split intein is the Ssp DnaE intein, which comprises two subunits, namely, DnaE-N and DnaE-C. The two different subunits are encoded by separate genes, namely dnaE-n and dnaE-c, which encode the DnaE-N and DnaE-C subunits, respectively. DnaE is a naturally occurring split intein in Synechocytis sp. PCC6803 and is capable of directing trans-splicing of two separate proteins, each comprising a fusion with either DnaE-N or DnaE-C.

Additional naturally occurring or engineered split-intein sequences are known in the or can be made from whole-intein sequences described herein or those available in the art. Examples of split-intein sequences can be found in Stevens et al., “A promiscuous split intein with expanded protein engineering applications,” PNAS, 2017, Vol. 114: 8538-8543; Iwai et al., “Highly efficient protein trans-splicing by a naturally split DnaE intein from Nostc punctiforme, FEBS Lett, 580: 1853-1858, each of which are incorporated herein by reference. Additional split intein sequences can be found, for example, in WO 2013/045632, WO 2014/055782, WO 2016/069774, and EP2877490, the contents each of which are incorporated herein by reference. In addition, protein splicing in trans has been described in vivo and in vitro (Shingledecker, et al., Gene 207:187 (1998), Southworth, et al., EMBO J. 17:918 (1998); Mills, et al., Proc. Natl. Acad. Sci. USA, 95:3543-3548 (1998); Lew, et al., J. Biol. Chem., 273:15887-15890 (1998); Wu, et al., Biochim. Biophys. Acta 35732:1 (1998b), Yamazaki, et al., J. Am. Chem. Soc. 120:5591 (1998), Evans, et al., J. Biol. Chem. 275:9091 (2000); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999)) and provides the opportunity to express a protein as to two inactive fragments that subsequently undergo ligation to form a functional product.

### RNA-Protein Interaction Domain

In various embodiments, two separate protein domains (e.g., a Cas9 domain and a polymerase domain) may be colocalized to one another to form a functional complex (akin to the function of a fusion protein comprising the two separate protein domains) by using an “RNA-protein recruitment system,” such as the “MS2 tagging technique.” Such systems generally tag one protein domain with an “RNA-protein interaction domain” (aka “RNA-protein recruitment domain”) and the other with an “RNA-binding protein” that specifically recognizes and binds to the RNA-protein interaction domain, e.g., a specific hairpin structure. These types of systems can be leveraged to colocalize the domains of a prime editor, as well as to recruitment additional functionalities to a prime editor, such as a UGI domain. In one example, the MS2 tagging technique is based on the interaction of the MS2 bacteriophage coat protein (“MCP” or “MS2cp”) with a stem-loop or hairpin structure present in the genome of the phage, i.e., the “MS2 hairpin.” In the case of the MS2 hairpin, it is recognized and bound by the MS2 bacteriophage coat protein (MCP). Thus, in one exemplary scenario a deaminase-MS2 fusion can recruit a Cas9-MCP fusion.

A review of other modular RNA-protein interaction domains are described in the art, for example, in Johansson et al., “RNA recognition by the MS2 phage coat protein,” Sen Virol., 1997, Vol. 8(3): 176-185; Delebecque et al., “Organization of intracellular reactions with rationally designed RNA assemblies,” Science, 2011, Vol. 333: 470-474; Mali et al., “Cas9 transcriptional activators for target specificity screening and paired nickases for cooperative genome engineering,” Nat. Biotechnol., 2013, Vol. 31: 833-838; and Zalatan et al., “Engineering complex synthetic transcriptional programs with CRISPR RNA scaffolds,” Cell, 2015, Vol. 160: 339-350, each of which are incorporated herein by reference in their entireties. Other systems include the PP7 hairpin, which specifically recruits the PCP protein, and the “com” hairpin, which specifically recruits the Com protein. See Zalatan et al.

The nucleotide sequence of the MS2 hairpin (or equivalently referred to as the “MS2 aptamer”) is: GCCAACATGAGGATCACCCATGTCTGCAGGGCC (SEQ ID NO: 166). The amino acid sequence of the MCP or MS2cp is:

(SEQ ID NO: 167)
GSASNFTQFVLVDNGGTGDVTVAPSNFANGVAEWISSNSRSQAYK
VTCSVRQSSAQNRKYTIKVEVPKVATQTVGGEELPVAGWRSYLNM
ELTIPIFATNSDCELIVKAMQGLLKDGNPIPSAIAANSGIY.

### E. UGI Domain

In other embodiments, the prime editors utilized in the methods and compositions described herein may comprise one or more uracil glycosylase inhibitor domains. The term “uracil glycosylase inhibitor (UGI)” or “UGI domain.” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 168. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 168. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 168. In some embodiments, a UGI comprises an amino acid sequence homologous to the amino acid sequence set forth in SEQ ID NO: 168, or an amino acid sequence homologous to a fragment of the amino acid sequence set forth in SEQ ID NO: 168. In some embodiments, proteins comprising UGI or fragments of UGI or homologs of UGI or UGI fragments are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 168. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 168. In some embodiments, the UGI comprises the following amino acid sequence:

Uracil-DNA glycosylase inhibitor:
>sp|P14739|UNGI_BPPB2
(SEQ ID NO: 168)
MTNLSDIIEKETGKQLVIQESILMLPEEVEEVIGNKPESDILVHT
AYDESTDENVMLLTSDAPEYKPWALVIQDSNGENKIKML.

The prime editors utilized in the methods and compositions described herein may comprise more than one UGI domain, which may be separated by one or more linkers as described herein.

### F. Additional PE Elements

In certain embodiments, the prime editors utilized in the methods and compositions described herein may comprise an inhibitor of base repair. The term “inhibitor of base repair” or “IBR” refers to a protein that is capable in inhibiting the activity of a nucleic acid repair enzyme, for example a base excision repair enzyme. In some embodiments, the IBR is an inhibitor of OGG base excision repair. In some embodiments, the IBR is an inhibitor of base excision repair (“iBER”). Exemplary inhibitors of base excision repair include inhibitors of APE1. Endo III. Endo IV, Endo V, Endo VIII, Fpg, hOGG1, hNEIL1, T7 EndoI, T4PDG, UDG, hSMUG1, and hAAG. In some embodiments, the IBR is an inhibitor of Endo V or hAAG. In some embodiments, the IBR is an iBER that may be a catalytically inactive glycosylase or catalytically inactive dioxygenase or a small molecule or peptide inhibitor of an oxidase, or variants thereof. In some embodiments, the IBR is an iBER that may be a TDG inhibitor, MBD4 inhibitor or an inhibitor of an AlkBH enzyme. In some embodiments, the IBR is an iBER that comprises a catalytically inactive TDG or catalytically inactive MBD4. An exemplary catalytically inactive TDG is an N140A mutant of SEQ ID NO: 172 (human TDG).

Some exemplary glycosylases are provided below. The catalytically inactivated variants of any of these glycosylase domains are iBERs that may be fused to the napDNAbp or polymerase domain of the prime editors utilized in the methods and compositions provided in this disclosure.

OGG (human)
(SEQ ID NO: 169)
MPARALLPRRMGHRTLASTPALWASIPCPRSELRLDLVLPSGQSF
RWREQSPAHWSGVLADQVWTLTQTEEQLHCTVYRGDKSQASRPTP
DELEAVRKYFQLDVTLAQLYHHWGSVDSHFQEVAQKFQGVRLLRQ
DPIECLFSFICSSNNNIARITGMVERLCQAFGPRLIQLDDVTYHG
FPSLQALAGPEVEAHLRKLGLGYRARYVSASARAILEEQGGLAWL
QQLRESSYEEAHKALCILPGVGTKVADCICLMALDKPQAVPVDVH
MWHIAQRDYSWHPTTSQAKGPSPQTNKELGNFFRSLWGPYAGWAQ
AVLFSADLRQSRHAQEPPAKRRKGSKGPEG
MPG (human)
(SEQ ID NO: 170)
MVTPALQMKKPKQFCRRMGQKKQRPARAGQPHSSSDAAQAPAEQP
HSSSDAAQAPCPRERCLGPPTTPGPYRSIYFSSPKGHLTRLGLEF
FDQPAVPLARAFLGQVLVRRLPNGTELRGRIVETEAYLGPEDEAA
HSRGGRQTPRNRGMFMKPGTLYVYIIYGMYFCMNISSQGDGACVL
LRALEPLEGLETMRQLRSTLRKGTASRVLKDRELCSGPSKLCQAL
AINKSFDQRDLAQDEAVWLERGPLEPSEPAVVAAARVGVGHAGEW
ARKPLRFYVRGSPWVSVVDRVAEQDTQA
MBD4 (human)
(SEQ ID NO: 171)
MGTTGLESLSLGDRGAAPTVTSSERLVPDPPNDLRKEDVAMELER
VGEDEEQMMIKRSSECNPLLQEPIASAQFGATAGTECRKSVPCGW
ERVVKQRLFGKTAGREDVYFISPQGLKFRSKSSLANYLHKNGETS
LKPEDFDFTVLSKRGIKSRYKDCSMAALTSHLQNQSNNSNWNLRT
RSKCKKDVFMPPSSSSELQESRGLSNFTSTHLLLKEDEGVDDVNF
RKVRKPKGKVTILKGIPIKKTKKGCRKSCSGFVQSDSKRESVCNK
ADAESEPVAQKSQLDRTVCISDAGACGETLSVTSEENSLVKKKER
SLSSGSNFCSEQKTSGIINKFCSAKDSEHNEKYEDTFLESEEIGT
KVEVVERKEHLHTDILKRGSEMDNNCSPTRKDFTGEKIFQEDTIP
RTQIERRKTSLYFSSKYNKEALSPPRRKAFKKWTPPRSPFNLVQE
TLFHDPWKLLIATIFLNRTSGKMAIPVLWKFLEKYPSAEVARTAD
WRDVSELLKPLGLYDLRAKTIVKFSDEYLTKQWKYPIELHGIGKY
GNDSYRIFCVNEWKQVHPEDHKLNKYHDWLWENHEKLSLS
TDG (human)
(SEQ ID NO: 172)
MEAENAGSYSLQQAQAFYTFPFQQLMAEAPNMAVVNEQQMPEEVP
APAPAQEPVQEAPKGRKRKPRTTEPKQPVEPKKPVESKKSGKSAK
SKEKQEKITDTFKVKRKVDRENGVSEAELLTKTLPDILTFNLDIV
IIGINPGLMAAYKGHHYPGPGNHFWKCLFMSGLSEVQLNHMDDHT
LPGKYGIGFTNMVERTTPGSKDLSSKEFREGGRILVQKLQKYQPR
IAVENGKCIYEIFSKEVFGVKVKNLEFGLQPHKIPDTETLCYVMP
SSSARCAQFPRAQDKVHYYIKLKDLRDQLKGIERNMDVQEVQYTF
DLQLAQEDAKKMAVKEEKYDPGYEAAYGGAYGENPCSSEPCGFSS
NGLIESVELRGESAFSGIPNGQWMTQSFTDQIPSFSNHCGTQEQE
EESHA

In some embodiments, the fusion proteins described herein may comprise one or more heterologous protein domains (e.g., about or more than about 1, 2, 3, 4, 5, 6, 7, 8, 9, 10, or more domains in addition to the prime editor components). A fusion protein may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags that are useful for solubilization, purification, or detection of the fusion proteins.

Examples of protein domains that may be fused to a prime editor or component thereof (e.g., the napDNAbp domain, the polymerase domain, or the NLS domain) include, without limitation, epitope tags, and reporter gene sequences. Non-limiting examples of epitope tags include histidine (His) tags, V5 tags. FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A prime editor may be fused to a gene sequence encoding a protein or a fragment of a protein that bind DNA molecules or bind other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a prime editor are described in US Patent Publication No. 2011/0059502, published Mar. 10, 2011 and incorporated herein by reference in its entirety.

In an aspect of the disclosure, a reporter gene which includes, but is not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In certain embodiments of the disclosure the gene product is luciferase. In a further embodiment of the disclosure the expression of the gene product is decreased.

Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments of the present disclosure, the activity of the prime editing system may be temporally regulated by adjusting the residence time, the amount, and/or the activity of the expressed components of the PE system. For example, as described herein, the PE may be fused with a protein domain that is capable of modifying the intracellular half-life of the PE. In certain embodiments involving two or more vectors (e.g., a vector system in which the components described herein are encoded on two or more separate vectors), the activity of the PE system may be temporally regulated by controlling the timing in which the vectors are delivered. For example, in some embodiments a vector encoding the nuclease system may deliver the PE prior to the vector encoding the template. In other embodiments, the vector encoding the PEgRNA may deliver the guide prior to the vector encoding the PE system. In some embodiments, the vectors encoding the PE system and PEgRNA are delivered simultaneously. In certain embodiments, the simultaneously delivered vectors temporally deliver, e.g., the PE, PEgRNA, and/or second strand guide RNA components. In further embodiments, the RNA (such as, e.g., the nuclease transcript) transcribed from the coding sequence on the vectors may further comprise at least one element that is capable of modifying the intracellular half-life of the RNA and/or modulating translational control. In some embodiments, the half-life of the RNA may be increased. In some embodiments, the half-life of the RNA may be decreased. In some embodiments, the element may be capable of increasing the stability of the RNA. In some embodiments, the element may be capable of decreasing the stability of the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription. In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus (WHP).

Posttranscriptional Regulatory Element (WPRE), which creates a tertiary structure to enhance expression from the transcript. In further embodiments, the element is a modified and/or truncated WPRE sequence that is capable of enhancing expression from the transcript, as described, for example in Zufferey et al., J Virol, 73(4): 2886-92 (1999) and Flajolet et al., J Virol, 72(7): 6175-80 (1998). In some embodiments, the WPRE or equivalent may be added to the 3′ UTR of the RNA. In some embodiments, the element may be selected from other RNA sequence motifs that are enriched in either fast- or slow-decaying transcripts. In some embodiments, the vector encoding the PE or the PEgRNA may be self-destroyed via cleavage of a target sequence present on the vector by the PE system. The cleavage may prevent continued transcription of a PE or a PEgRNA from the vector. Although transcription may occur on the linearized vector for some amount of time, the expressed transcripts or proteins subject to intracellular degradation will have less time to produce off-target effects without continued supply from expression of the encoding vectors.

### PEgRNAs

The prime editing system utilized in the methods and compositions described herein contemplates the use of any suitable PEgRNAs.

### PEgRNA Architecture

In some embodiments, an extended guide RNA usable in the prime editing system utilized in the methods and compositions disclosed herein whereby a traditional guide RNA includes a ˜20 nt protospacer sequence and a gRNA core region, which binds with the napDNAbp. In this embodiment, the guide RNA includes an extended RNA segment at the 5′ end, i.e., a 5′ extension. In this embodiment, the 5′extension includes a reverse transcription template sequence, a reverse transcription primer binding site, and an optional 5-20 nucleotide linker sequence. The RT primer binding site hybridizes to the free 3′ end that is formed after a nick is formed in the non-target strand of the R-loop, thereby priming reverse transcriptase for DNA polymerization in the 5′-3′ direction.

In another embodiment, an extended guide RNA usable in the prime editing system utilized in the methods and compositions disclosed herein whereby a traditional guide RNA includes a ˜20 nt protospacer sequence and a gRNA core, which binds with the napDNAbp. In this embodiment, the guide RNA includes an extended RNA segment at the 3′ end, i.e., a 3′ extension. In this embodiment, the 3′extension includes a reverse transcription template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

In another embodiment, an extend guide RNA usable in the prime editing system utilized in the methods and compositions disclosed herein whereby a traditional guide RNA includes a ˜20 nt protospacer sequence and a gRNA core, which binds with the napDNAbp. In this embodiment, the guide RNA includes an extended RNA segment at an intermolecular position within the gRNA core, i.e., an intramolecular extension. In this embodiment, the intramolecular extension includes a reverse transcription template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

In one embodiment, the position of the intermolecular RNA extension is not in the protospacer sequence of the guide RNA. In another embodiment, the position of the intermolecular RNA extension in the gRNA core. In still another embodiment, the position of the intermolecular RNA extension is any with the guide RNA molecule except within the protospacer sequence, or at a position which disrupts the protospacer sequence. In one embodiment, the intermolecular RNA extension is inserted downstream from the 3′ end of the protospacer sequence. In another embodiment, the intermolecular RNA extension is inserted at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides downstream of the 3′ end of the protospacer sequence.

In other embodiments, the intermolecular RNA extension is inserted into the gRNA, which refers to the portion of the guide RNA corresponding or comprising the tracrRNA, which binds and/or interacts with the Cas9 protein or equivalent thereof (i.e., a different napDNAbp). Preferably the insertion of the intermolecular RNA extension does not disrupt or minimally disrupts the interaction between the tracrRNA portion and the napDNAbp.

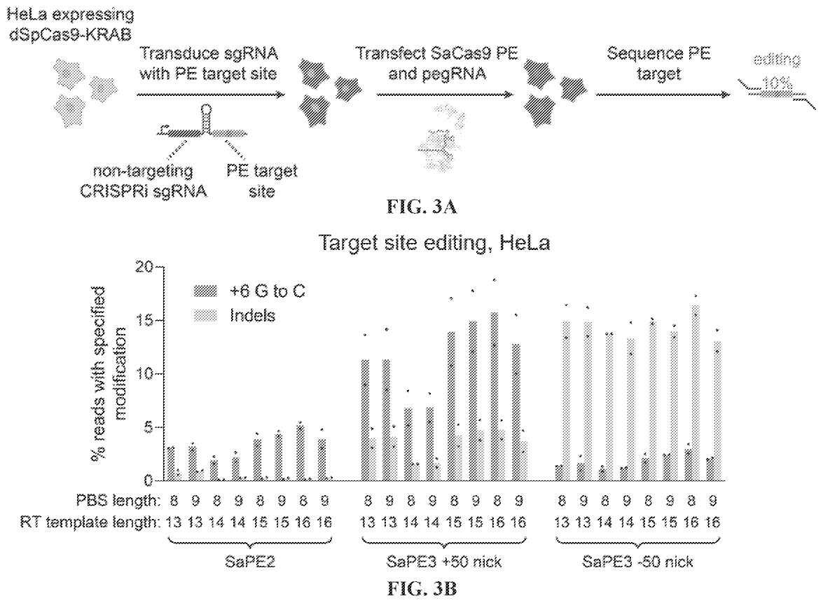

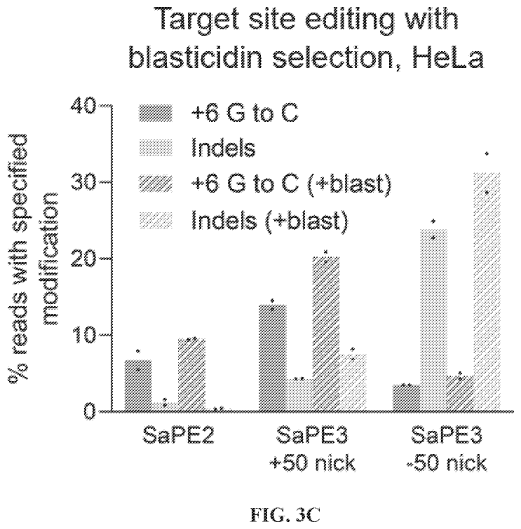

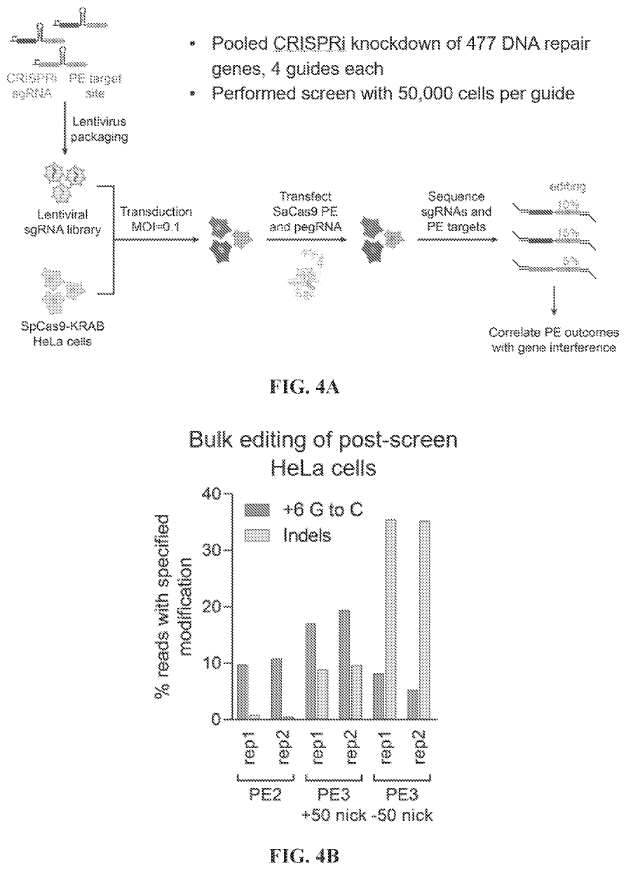

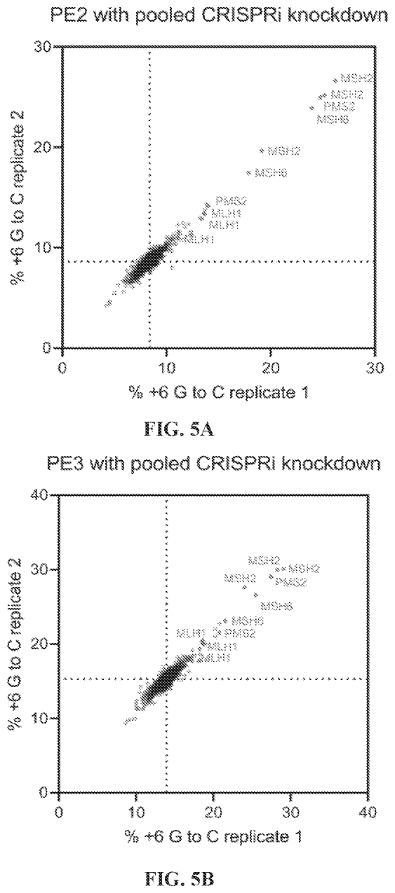

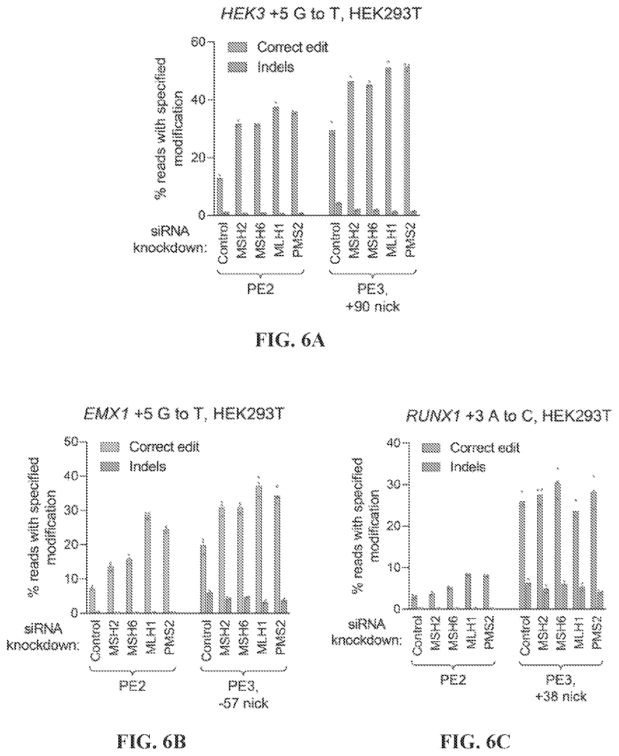

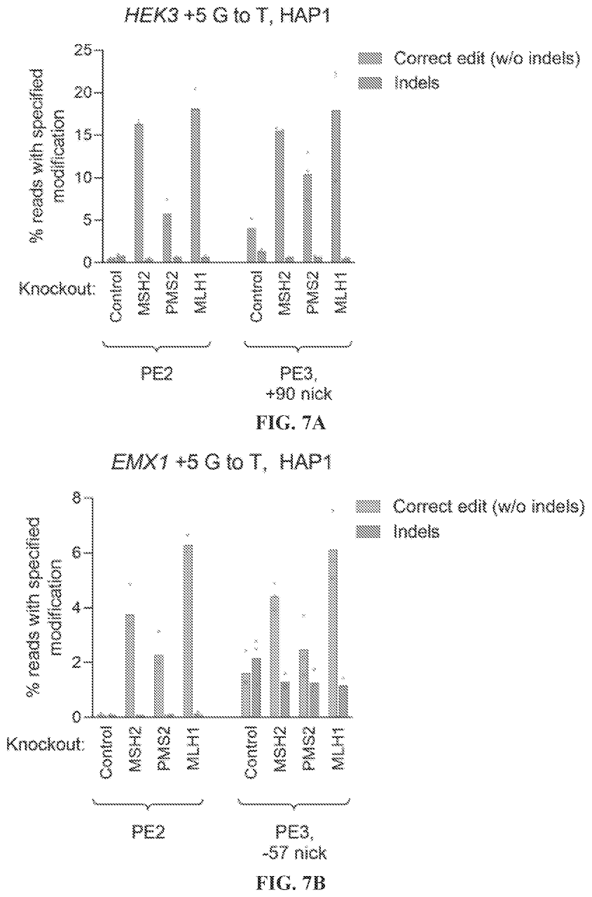

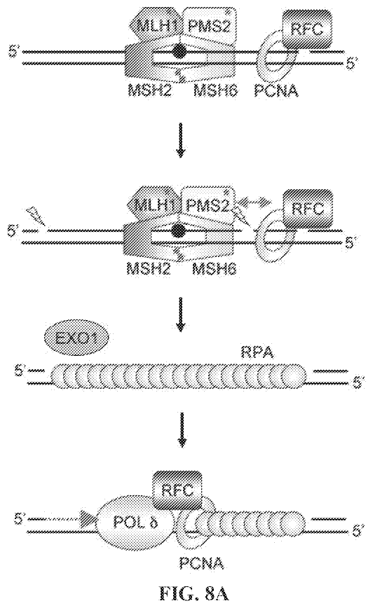

The length of the RNA extension (which includes at least the RT template and primer binding site, e.g., see FIG. 3) can be any useful length. In various embodiments, the RNA extension is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence can also be any suitable length. For example, the RT template sequence can be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In still other embodiments, wherein the reverse transcription primer binding site sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In other embodiments, the optional linker or spacer sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence, in certain embodiments, encodes a single-stranded DNA molecule which is homologous to the non-target strand (and thus, complementary to the corresponding site of the target strand) but includes one or more nucleotide changes. The least one nucleotide change may include one or more single-base nucleotide changes, one or more deletions, and one or more insertions.

The synthesized single-stranded DNA product of the RT template sequence is homologous to the non-target strand and contains one or more nucleotide changes. The single-stranded DNA product of the RT template sequence hybridizes in equilibrium with the complementary target strand sequence, thereby displacing the homologous endogenous target strand sequence. The displaced endogenous strand may be referred to in some embodiments as a 5′ endogenous DNA flap species. This 5′ endogenous DNA flap species can be removed by a 5′ flap endonuclease (e.g., FEN1) and the single-stranded DNA product, now hybridized to the endogenous target strand, may be ligated, thereby creating a mismatch between the endogenous sequence and the newly synthesized strand. The mismatch may be resolved by the cell's innate DNA repair and/or replication processes.

In various embodiments, the nucleotide sequence of the RT template sequence corresponds to the nucleotide sequence of the non-target strand which becomes displaced as the 5′ flap species and which overlaps with the site to be edited.

In various embodiments of the extended guide RNAs, the reverse transcription template sequence may encode a single-strand DNA flap that is complementary to an endogenous DNA sequence adjacent to a nick site, wherein the single-strand DNA flap comprises a desired nucleotide change. The single-stranded DNA flap may displace an endogenous single-strand DNA at the nick site. The displaced endogenous single-strand DNA at the nick site can have a 5′ end and form an endogenous flap, which can be excised by the cell. In various embodiments, excision of the 5′ end endogenous flap can help drive product formation since removing the 5′ end endogenous flap encourages hybridization of the single-strand 3′ DNA flap to the corresponding complementary DNA strand, and the incorporation or assimilation of the desired nucleotide change carried by the single-strand 3′ DNA flap into the target DNA.

In various embodiments of the extended guide RNAs, the cellular repair of the single-strand DNA flap results in installation of the desired nucleotide change, thereby forming a desired product.

In still other embodiments, the desired nucleotide change is installed in an editing window that is between about −5 to +5 of the nick site, or between about −10 to +10 of the nick site, or between about −20 to +20 of the nick site, or between about −30 to +30 of the nick site, or between about −40 to +40 of the nick site, or between about −50 to +50 of the nick site, or between about −60 to +60 of the nick site, or between about −70 to +70 of the nick site, or between about −80 to +80 of the nick site, or between about −90 to +90 of the nick site, or between about −100 to +100 of the nick site, or between about −200 to +200 of the nick site.

In other embodiments, the desired nucleotide change is installed in an editing window that is between about +1 to +2 from the nick site, or about +1 to +3, +1 to +4, +1 to +5, +1 to +6, +1 to +7, +1 to +8, +1 to +9, +1 to +10, +1 to +11, +1 to +12, +l to +13, +1 to +14, +1 to +15, +1 to +16, +1 to +17, +1 to +18, +1 to +19, +1 to +20, +1 to +21, +1 to +22, +1 to +23, +1 to +24, +1 to +25, +1 to +26, +1 to +27, +1 to +28, +1 to +29, +1 to +30, +1 to +31, +1 to +32, +1 to +33, +1 to +34, +1 to +35, +1 to +36, +1 to +37, +1 to +38, +1 to +39, +1 to +40, +1 to +41, +1 to +42, +1 to +43, +1 to +44, +1 to +45, +1 to +46, +1 to +47, +1 to +48, +1 to +49, +1 to +50, +1 to +51, +1 to +52, +1 to +53, +1 to +54, +1 to +55, +1 to +56, +1 to +57, +1 to +58, +1 to +59, +1 to +60, +1 to +61, +1 to +62, +1 to +63, +1 to +64, +1 to +65, +1 to +66, +1 to +67, +1 to +68, +1 to +69, +1 to +70, +1 to +71, +1 to +72, +1 to +73, +1 to +74, +1 to +75, +1 to +76, +1 to +77, +1 to +78, +1 to +79, +1 to +80, +1 to +81, +1 to +82, +1 to +83, +1 to +84, +1 to +85, +1 to +86, +1 to +87, +1 to +88, +1 to +89, +1 to +90, +1 to +90, +1 to +91, +1 to +92, +1 to +93, +1 to +94, +1 to +95, +1 to +96, +1 to +97, +1 to +98, +1 to +99, +1 to +100, +1 to +101, +1 to +102, +1 to +103, +1 to +104, +1 to +105, +1 to +106, +1 to +107, +1 to +108, +1 to +109, +1 to +110, +1 to +111, +1 to +112, +1 to +113, +1 to +114, +1 to +115, +1 to +116, +1 to +117, +1 to +118, +1 to +119, +1 to +120, +1 to +121, +1 to +122, +1 to +123, +1 to +124, or +1 to +125 from the nick site.

In still other embodiments, the desired nucleotide change is installed in an editing window that is between about +1 to +2 from the nick site, or about +1 to +5, +1 to +10, +1 to +15, +1 to +20, +1 to +25, +1 to +30, +1 to +35, +1 to +40, +1 to +45, +1 to +50, +1 to +55, +1 to +100, +1 to +105, +1 to +110, +1 to +115, +1 to +120, +1 to +125, +1 to +130, +1 to +135, +1 to +140, +1 to +145, +1 to +150, +1 to +155, +1 to +160, +1 to +165, +1 to +170, +1 to +175, +1 to +180, +1 to +185, +1 to +190, +1 to +195, or +1 to +200, from the nick site.

In various aspects, the extended guide RNAs are modified versions of a guide RNA. Guide RNAs maybe naturally occurring, expressed from an encoding nucleic acid, or synthesized chemically. Methods are well known in the art for obtaining or otherwise synthesizing guide RNAs and for determining the appropriate sequence of the guide RNA, including the protospacer sequence which interacts and hybridizes with the target strand of a genomic target site of interest.

In various embodiments, the particular design aspects of a guide RNA sequence will depend upon the nucleotide sequence of a genomic target site of interest (i.e., the desired site to be edited) and the type of napDNAbp (e.g., Cas9 protein) present in the prime editing systems utilized in the methods and compositions described herein, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT. Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a prime editor to a target sequence may be assessed by any suitable assay. For example, the components of a prime editor, including the guide sequence to be tested, may be provided to a host cell having the corresponding target sequence, such as by transfection with vectors encoding the components of a prime editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a prime editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG (SEQ ID NO: 173) where NNNNNNNNNNNNXGG (SEQ ID NO: 174) (N is A, G, T, or C; and X can be anything). A unique target sequence in a genome may include an S. pyogenes Cas9 target site of the form MMMMMMMMMNNNNNNNNNNNXGG (SEQ ID NO: 175) where NNNNNNNNNNNXGG (SEQ ID NO: 176) (N is A, G. T. or C; and X can be anything). For the S. thermophilus CRISPR1Cas9, a unique target sequence in a genome may include a Cas9 target site of the form MMMMMMMMNNNNNNNNNNNNXXAGAAW (SEQ ID NO: 177) where NNNNNNNNNNNNXXAGAAW (SEQ ID NO: 178) (N is A, G, T. or C; X can be anything; and W is A or T). A unique target sequence in a genome may include an S. thermophilus CRISPR 1 Cas9 target site of the form MMMMMMMMMNNNNNNNNNNNXXAGAAW (SEQ ID NO: 179) where NNNNNNNNNNNXXAGAAW (SEQ ID NO: 180) (N is A, G. T, or C; X can be anything; and W is A or T). For the S. pyogenes Cas9, a unique target sequence in a genome may include a Cas9 target site of the form MMMMMMMMNNNNNNNNNNNNXGGXG (SEQ ID NO: 181) where NNNNNNNNNNNNXGGXG (SEQ ID NO: 182) (N is A, G, T, or C; and X can be anything). A unique target sequence in a genome may include an S. pyogenes Cas9 target site of the form MMMMMMMMMNNNNNNNNNNNXGGXG (SEQ ID NO: 183) where NNNNNNNNNNNXGGXG (SEQ ID NO: 184) (N is A, G, T, or C; and X can be anything). In each of these sequences “M” may be A, G, T, or C, and need not be considered in identifying a sequence as unique.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62). Further algorithms may be found in U.S. application Ser. No. 61/836,080; Broad Reference BI-2013/004A); incorporated herein by reference.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In some embodiments, the single transcript further includes a transcription termination sequence: preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(1)
(SEQ ID NO: 185)
NNNNNNNNGTTTTTGTACTCTCAAGATTTAGAAATAAATCTTGCA
GAAGCTACAAAGATAAGGCTTCATGCCGAAATCAACACCCTGTCA
TTTTATGGCAGGGTGTTTTCGTTATTTAATTTTTT;
(2)
(SEQ ID NO: 186)
NNNNNNNNNNNNNNNNNNGTTTTTGTACTCTCAGAAATGCAGAAG
CTACAAAGATAAGGCTTCATGCCGAAATCAACACCCTGTCATTTT
ATGGCAGGGTGTTTTCGTTATTTAATTTTTT;
(3)
(SEQ ID NO: 187)
NNNNNNNNNNNNNNNNNNNNGTTTTTGTACTCTCAGAAATGCAGA
AGCTACAAAGATAAGGCTTCATGCCGAAATCAACACCCTGTCATT
TTATGGCAGGGTGTTTTTT;
(4)
(SEQ ID NO: 188)
NNNNNNNNNNNNNNNNNNNNGTTTTAGAGCTAGAAATAGCAAGTT
AAAATAAGGCTAGTCCGTTATCAACTTGAAAAAGTGGCACCGAGT
CGGTGCTTTTTT;
(5)
(SEQ ID NO: 189)
NNNNNNNNNNNNNNNNNNNNGTTTTAGAGCTAGAAATAGCAAGTT
AAAATAAGGCTAGTCCGTTATCAACTTGAAAAAGTGTTTTTTT;
and
(6)
(SEQ ID NO: 190)
NNNNNNNNNNNNNNNNNNNNGTTTTAGAGCTAGAAATAGCAAGTT
AAAATAAGGCTAGTCCGTTATCATTTTTTTT.

In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the fusion proteins comprising a Cas9 domain and a single-stranded DNA binding protein, as disclosed herein, to a target site, e.g., a site comprising a point mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein.

In some embodiments, the guide RNA comprises a structure 5′-[guide sequence]-GUUUUAGAGCUAGAAAUAGCAAGUUAAAAUAAAGGCUAGUCCGUUAUCAACUUG AAAAAGUGGCACCGAGUCGGUGCUUUUU-3′ (SEQ ID NO: 191), wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein. Additional guide sequences are well known in the art and can be used with the prime editors utilized in the methods and compositions described herein.

In some embodiments, a PEgRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: a homology arm, an editing template, and a primer binding site. In addition, the PEgRNA may comprise an optional 3′ end modifier region (e1) and an optional 5′ end modifier region (e2). Still further, the PEgRNA may comprise a transcriptional termination signal at the 3′ end of the PEgRNA (not depicted). These structural elements are further defined herein. The depiction of the structure of the PEgRNA is not meant to be limiting and embraces variations in the arrangement of the elements. For example, the optional sequence modifiers (e1) and (e2) could be positioned within or between any of the other regions shown, and not limited to being located at the 3′ and 5′ ends.

In some embodiments, a PEgRNA contemplated herein and may be designed in accordance with the methodology defined in Example 2. The PEgRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: a homology arm, an editing template, and a primer binding site. In addition, the PEgRNA may comprise an optional 3′ end modifier region (e1) and an optional 5′ end modifier region (e2). Still further, the PEgRNA may comprise a transcriptional termination signal on the 3′ end of the PEgRNA (not depicted). These structural elements are further defined herein. The depiction of the structure of the PEgRNA is not meant to be limiting and embraces variations in the arrangement of the elements. For example, the optional sequence modifiers (e1) and (e2) could be positioned within or between any of the other regions shown, and not limited to being located at the 3′ and 5′ ends.

### PEgRNA Improvements

The PEgRNAs may also include additional design improvements that may modify the properties and/or characteristics of PEgRNAs thereby improving the efficacy of prime editing. In various embodiments, these improvements may belong to one or more of a number of different categories, including but not limited to: (1) designs to enable efficient expression of functional PEgRNAs from non-polymerase III (pol III) promoters, which would enable the expression of longer PEgRNAs without burdensome sequence requirements; (2) improvements to the core, Cas9-binding PEgRNA scaffold, which could improve efficacy; (3) modifications to the PEgRNA to improve RT processivity, enabling the insertion of longer sequences at targeted genomic loci; and (4) addition of RNA motifs to the 5′ or 3′ termini of the PEgRNA that improve PEgRNA stability, enhance RT processivity, prevent misfolding of the PEgRNA, or recruit additional factors important for genome editing.

In one embodiment, PEgRNA could be designed with polIII promoters to improve the expression of longer-length PEgRNA with larger extension arms. sgRNAs are typically expressed from the U6 snRNA promoter. This promoter recruits pol III to express the associated RNA and is useful for expression of short RNAs that are retained within the nucleus. However, pol III is not highly processive and is unable to express RNAs longer than a few hundred nucleotides in length at the levels required for efficient genome editing. Additionally, pol III can stall or terminate at stretches of U's, potentially limiting the sequence diversity that could be inserted using a PEgRNA. Other promoters that recruit polymerase II (such as pCMV) or polymerase I (such as the U1 snRNA promoter) have been examined for their ability to express longer sgRNAs. However, these promoters are typically partially transcribed, which would result in extra sequence 5′ of the spacer in the expressed PEgRNA, which has been shown to result in markedly reduced Cas9:sgRNA activity in a site-dependent manner. Additionally, while pol III-transcribed PEgRNAs can simply terminate in a run of 6-7 U's, PEgRNAs transcribed from pol II or pol I would require a different termination signal. Often such signals also result in polyadenylation, which would result in undesired transport of the PEgRNA from the nucleus. Similarly, RNAs expressed from pol II promoters such as pCMV are typically 5′-capped, also resulting in their nuclear export.

Previously, Rinn and coworkers screened a variety of expression platforms for the production of long-noncoding RNA-(lncRNA) tagged sgRNAs¹⁸³. These platforms include RNAs expressed from pCMV and that terminate in the ENE element from the MALAT1 ncRNA from humans¹⁸⁴, the PAN ENE element from KSHV¹⁸, or the 3′ box from U1 snRNA¹⁸⁶. Notably, the MALAT1 ncRNA and PAN ENEs form triple helices protecting the polyA-tail^{184, 187}. These constructs could also enhance RNA stability. It is contemplated that these expression systems will also enable the expression of longer PEgRNAs.

In addition, a series of methods have been designed for the cleavage of the portion of the pol II promoter that would be transcribed as part of the PEgRNA, adding either a self-cleaving ribozyme such as the hammerhead¹⁸⁸, pistol¹⁸⁹, hatchet¹⁸⁹, hairpin¹⁹⁰, VS¹⁹¹, twister¹⁹², or twister sister¹⁹² ribozymes, or other self-cleaving elements to process the transcribed guide, or a hairpin that is recognized by Csy4¹⁹³ and also leads to processing of the guide. Also, it is hypothesized that incorporation of multiple ENE motifs could lead to improved PEgRNA expression and stability, as previously demonstrated for the KSHV PAN RNA and element¹⁸⁵. It is also anticipated that circularizing the PEgRNA in the form of a circular intronic RNA (ciRNA) could also lead to enhanced RNA expression and stability, as well as nuclear localization¹⁹⁴.

In various embodiments, the PEgRNA may include various above elements, as exemplified by the following sequence.

Non-limiting example 1-PERNA expression
platform consisting of pCMV, Csy4
hairpin, the PEgRNA, and MALATI ENE
(SEQ ID NO: 192)
TAGTTATTAATAGTAATCAATTACGGGGTCATTAGTTCATAGCCC
ATATATGGAGTTCCGCGTTACATAACTTACGGTAAATGGCCCGCC
TGGCTGACCGCCCAACGACCCCCGCCCATTGACGTCAATAATGAC
GTATGTTCCCATAGTAACGCCAATAGGGACTTTCCATTGACGTCA
ATGGGTGGAGTATTTACGGTAAACTGCCCACTTGGCAGTACATCA
AGTGTATCATATGCCAAGTACGCCCCCTATTGACGTCAATGACGG
TAAATGGCCCGCCTGGCATTATGCCCAGTACATGACCTTATGGGA
CTTTCCTACTTGGCAGTACATCTACGTATTAGTCATCGCTATTAC
CATGGTGATGCGGTTTTGGCAGTACATCAATGGGCGTGGATAGCG
GTTTGACTCACGGGGATTTCCAAGTCTCCACCCCATTGACGTCAA
TGGGAGTTTGTTTTGGCACCAAAATCAACGGGACTTTCCAAAATG
TCGTAACAACTCCGCCCCATTGACGCAAATGGGCGGTAGGCGTGT
ACGGTGGGAGGTCTATATAAGCAGAGCTGGTTTAGTGAACCGTCA
GATCGTTCACTGCCGTATAGGCAGGGCCCAGACTGAGCACGTGAG
TTTTAGAGCTAGAAATAGCAAGTTAAAATAAGGCTAGTCCGTTAT
CAACTTGAAAAAGTGGGACCGAGTCGGTCCTCTGCCATCAAAGCG
TGCTCAGTCTGTTTTAGGGTCATGAAGGTTTTTCTTTTCCTGAGA
AAACAACACGTATTGTTTTCTCAGGTTTTGCTTTTTGGCCTTTTT
CTAGCTTAAAAAAAAAAAAAGCAAAAGATGCTGGTGGTTGGCACT
CCTGGTTTCCAGGACGGGGTTCAAATCCCTGCGGCGTCTTTGCTT
TGACT
Non-limiting example 2-PEgRNA expression
platform consisting of pCMV, Csy4
hairpin, the PEgRNA, and PAN ENE
(SEQ ID NO: 193)
TAGTTATTAATAGTAATCAATTACGGGGTCATTAGTTCATAGCCCA
TATATGGAGTTCCGCGTTACATAACTTACGGTAAATGGCCCGCCT
GGCTGACCGCCCAACGACCCCCGCCCATTGACGTCAATAATGACG
TATGTTCCCATAGTAACGCCAATAGGGACTTTCCATTGACGTCAA
TGGGTGGAGTATTTACGGTAAACTGCCCACTTGGCAGTACATCAA
GTGTATCATATGCCAAGTACGCCCCCTATTGACGTCAATGACGGT
AAATGGCCCGCCTGGCATTATGCCCAGTACATGACCTTATGGGAC
TTTCCTACTTGGCAGTACATCTACGTATTAGTCATCGCTATTACC
ATGGTGATGCGGTTTTGGCAGTACATCAATGGGCGTGGATAGCGG
TTTGACTCACGGGGATTTCCAAGTCTCCACCCCATTGACGTCAAT
GGGAGTTTGTTTTGGCACCAAAATCAACGGGACTTTCCAAAATGT
CGTAACAACTCCGCCCCATTGACGCAAATGGGCGGTAGGCGTGTA
CGGTGGGAGGTCTATATAAGCAGAGCTGGTTTAGTGAACCGTCAG
ATCGTTCACTGCCGTATAGGCAGGGCCCAGACTGAGCACGTGAGT
TTTAGAGCTAGAAATAGCAAGTTAAAATAAGGCTAGTCCGTTATC
AACTTGAAAAAGTGGGACCGAGTCGGTCCTCTGCCATCAAAGCGT
GCTCAGTCTGTTTTGTTTTGGCTGGGTTTTTCCTTGTTCGCACCG
GACACCTCCAGTGACCAGACGGCAAGGTTTTTATCCCAGTGTATA
TTGGAAAAACATGTTATACTTTTGACAATTTAACGTGCCTAGAGC
TCAAATTAAACTAATACCATAACGTAATGCAACTTACAACATAAA
TAAAGGTCAATGTTTAATCCATAAAAAAAAAAAAAAAAAAA
Non-limiting example 3-PBgRNA expression
platform consisting of pCMV, Csy4
hairpin, the PEgRNA, and 3xPAN ENE
(SEQ ID NO: 194)
TAGTTATTAATAGTAATCAATTACGGGGTCATTAGTTCATAGCCC
ATATATGGAGTTCCGCGTTACATAACTTACGGTAAATGGCCCGCC
TGGCTGACCGCCCAACGACCCCCGCCCATTGACGTCAATAATGAC
GTATGTTCCCATAGTAACGCCAATAGGGACTTTCCATTGACGTCA
ATGGGTGGAGTATTTACGGTAAACTGCCCACTTGGCAGTACATCA
AGTGTATCATATGCCAAGTACGCCCCCTATTGACGTCAATGACGG
TAAATGGCCCGCCTGGCATTATGCCCAGTACATGACCTTATGGGA
CTTTCCTACTTGGCAGTACATCTACGTATTAGTCATCGCTATTAC
CATGGTGATGCGGTTTTGGCAGTACATCAATGGGCGTGGATAGCG
GTTTGACTCACGGGGATTTCCAAGTCTCCACCCCATTGACGTCAA
TGGGAGTTTGTTTTGGCACCAAAATCAACGGGACTTTCCAAAATG
TCGTAACAACTCCGCCCCATTGACGCAAATGGGCGGTAGGCGTGT
ACGGTGGGAGGTCTATATAAGCAGAGCTGGTTTAGTGAACCGTCA
GATCGTTCACTGCCGTATAGGCAGGGCCCAGACTGAGCACGTGAG
TTTTAGAGCTAGAAATAGCAAGTTAAAATAAGGCTAGTCCGTTAT
CAACTTGAAAAAGTGGGACCGAGTCGGTCCTCTGCCATCAAAGCG
TGCTCAGTCTGTTTTGTTTTGGCTGGGTTTTTCCTTGTTCGCACC
GGACACCTCCAGTGACCAGACGGCAAGGTTTTTATCCCAGTGTAT
ATTGGAAAAACATGTTATACTTTTGACAATTTAACGTGCCTAGAG
CTCAAATTAAACTAATACCATAACGTAATGCAACTTACAACATAA
ATAAAGGTCAATGTTTAATCCATAAAAAAAAAAAAAAAAAAAACA
CACTGTTTTGGCTGGGTTTTTCCTTGTTCGCACCGGACACCTCCA
GTGACCAGACGGCAAGGTTTTTATCCCAGTGTATATTGGAAAAAC
ATGTTATACTTTTGACAATTTAACGTGCCTAGAGCTCAAATTAAA
CTAATACCATAACGTAATGCAACTTACAACATAAATAAAGGTCAA
TGTTTAATCCATAAAAAAAAAAAAAAAAAAATCTCTCTGTTTTGG
CTGGGTTTTTCCTTGTTCGCACCGGACACCTCCAGTGACCAGACG
GCAAGGTTTTTATCCCAGTGTATATTGGAAAAACATGTTATACTT
TTGACAATTTAACGTGCCTAGAGCTCAAATTAAACTAATACCATA
ACGTAATGCAACTTACAACATAAATAAAGGTCAATGTTTAATCCA
TAAAAAAAAAAAAAAAAAAA
Non-limiting example 4-PEgRNA expression
platform consisting of pCMV, Csy4
hairpin, the PEgRNA, and 3′ box
(SEQ ID NO: 195)
TAGTTATTAATAGTAATCAATTACGGGGTCATTAGTTCATAGCCC
ATATATGGAGTTCCGCGTTACATAACTTACGGTAAATGGCCCGCC
TGGCTGACCGCCCAACGACCCCCGCCCATTGACGTCAATAATGAC
GTATGTTCCCATAGTAACGCCAATAGGGACTTTCCATTGACGTCA
ATGGGTGGAGTATTTACGGTAAACTGCCCACTTGGCAGTACATCA
AGTGTATCATATGCCAAGTACGCCCCCTATTGACGTCAATGACGG
TAAATGGCCCGCCTGGCATTATGCCCAGTACATGACCTTATGGGA
CTTTCCTACTTGGCAGTACATCTACGTATTAGTCATCGCTATTAC
CATGGTGATGCGGTTTTGGCAGTACATCAATGGGCGTGGATAGCG
GTTTGACTCACGGGGATTTCCAAGTCTCCACCCCATTGACGTCAA
TGGGAGTTTGTTTTGGCACCAAAATCAACGGGACTTTCCAAAATG
TCGTAACAACTCCGCCCCATTGACGCAAATGGGCGGTAGGCGTGT
ACGGTGGGAGGTCTATATAAGCAGAGCTGGTTTAGTGAACCGTCA
GATCGTTCACTGCCGTATAGGCAGGGCCCAGACTGAGCACGTGAG
TTTTAGAGCTAGAAATAGCAAGTTAAAATAAGGCTAGTCCGTTAT
CAACTTGAAAAAGTGGGACCGAGTCGGTCCTCTGCCATCAAAGCG
TGCTCAGTCTGTTTGTTTCAAAAGTAGACTGTACGCTAAGGGTCA
TATCTTTTTTTGTTTGGTTTGTGTCTTGGTTGGCGTCTTAAA
Non-limiting example 5-PEgRNA expression
platform consisting of pU1, Csy4 hairpin,
the PEgRNA, and 3′ box
(SEQ ID NO: 196)
CTAAGGACCAGCTTCTTTGGGAGAGAACAGACGCAGGGGGGGGAG
GGAAAAAGGGAGAGGCAGACGTCACTTCCCCTTGGCGGCTCTGGC
AGCAGATTGGTCGGTTGAGTGGCAGAAAGGCAGACGGGGACTGGG
CAAGGCACTGTCGGTGACATCACGGACAGGGCGACTTCTATGTAG
ATGAGGCAGCGCAGAGGCTGCTGCTTCGCCACTTGCTGCTTCACC
ACGAAGGAGTTCCCGTGCCCTGGGAGCGGGTTCAGGACCGCTGAT
CGGAAGTGAGAATCCCAGCTGTGTGTCAGGGCTGGAAAGGGCTCG
GGAGTGCGCGGGGCAAGTGACCGTGTGTGTAAAGAGTGAGGCGTA
TGAGGCTGTGTCGGGGCAGAGGCCCAAGATCTCAGTTCACTGCCG
TATAGGCAGGGCCCAGACTGAGCACGTGAGTTTTAGAGCTAGAAA
TAGCAAGTTAAAATAAGGCTAGTCCGTTATCAACTTGAAAAAGTG
GGACCGAGTCGGTCCTCTGCCATCAAAGCGTGCTCAGTCTGTTTC
AGCAAGTTCAGAGAAATCTGAACTTGCTGGATTTTTGGAGCAGGG
AGATGGAATAGGAGCTTGCTCCGTCCACTCCACGCATCGACCTGG
TATTGCAGTACCTCCAGGAACGGTGCACCCACTTTCTGGAGTTTC
AAAAGTAGACTGTACGCTAAGGGTCATATCTTTTTTTGTTTGGTT
TGTGTCTTGGTTGGCGTCTTAAA.

In various other embodiments, the PEgRNA may be improved by introducing improvements to the scaffold or core sequences. This can be done by introducing known The core, Cas9-binding PEgRNA scaffold can likely be improved to enhance PE activity. Several such approaches have already been demonstrated. For instance, the first pairing element of the scaffold (P1) contains a GTTTT-AAAAC (SEQ ID NO: 68) pairing element. Such runs of Ts have been shown to result in pol IU pausing and premature termination of the RNA transcript. Rational mutation of one of the TA pairs to a G-C pair in this portion of P1 has been shown to enhance sgRNA activity, suggesting this approach would also be feasible for PEgRNAs¹⁹⁵. Additionally, increasing the length of P1 has also been shown to enhance sgRNA folding and lead to improved activity¹⁹⁵, suggesting it as another avenue for the improvement of PEgRNA activity. Example improvements to the core can include:

PEgRNA containing a 6 nt extension to P1
(SEQ ID NO: 197)
GGCCCAGACTGAGCACGTGAGTTTTAGAGCTAGCTCATGAAAATG
AGCTAGCAAGTTAAAATAAGGCTAGTCCGTTATCAACTTGAAAAA
GTGGGACCGAGTCGGTCCTCTGCCATCAAAGCGTGCTCAGTCTGT
TTTTTT
PEgRNA containing a T-A to G-C mutation
within P1
(SEQ ID NO: 198)
GGCCCAGACTGAGCACGTGAGTTTGAGAGCTAGAAATAGCAAGTT
TAAATAAGGCTAGTCCGTTATCAACTTGAAAAAGTGGGACCGAGT
CGGTCCTCTGCCATCAAAGCGTGCTCAGTCTGTTTTTTT

In various other embodiments, the PEgRNA may be improved by introducing modifications to the edit template region. As the size of the insertion templated by the PEgRNA increases, it is more likely to be degraded by endonucleases, undergo spontaneous hydrolysis, or fold into secondary structures unable to be reverse-transcribed by the RT or that disrupt folding of the PEgRNA scaffold and subsequent Cas9-RT binding. Accordingly, it is likely that modification to the template of the PEgRNA might be necessary to affect large insertions, such as the insertion of whole genes. Some strategies to do so include the incorporation of modified nucleotides within a synthetic or semi-synthetic PEgRNA that render the RNA more resistant to degradation or hydrolysis or less likely to adopt inhibitory secondary structures¹⁹⁶. Such modifications could include 8-aza-7-deazaguanosine, which would reduce RNA secondary structure in G-rich sequences; locked-nucleic acids (LNA) that reduce degradation and enhance certain kinds of RNA secondary structure; 2′-O-methyl, 2′-fluoro, or 2′-O-methoxyethoxy modifications that enhance RNA stability. Such modifications could also be included elsewhere in the PEgRNA to enhance stability and activity. Alternatively or additionally, the template of the PEgRNA could be designed such that it both encodes for a desired protein product and is also more likely to adopt simple secondary structures that are able to be unfolded by the RT. Such simple structures would act as a thermodynamic sink, making it less likely that more complicated structures that would prevent reverse transcription would occur. Finally, one could also split the template into two, separate PEgRNAs. In such a design, a PE would be used to initiate transcription and also recruit a separate template RNA to the targeted site via an RNA-binding protein fused to Cas9 or an RNA recognition element on the PEgRNA itself such as the MS2 aptamer. The RT could either directly bind to this separate template RNA, or initiate reverse transcription on the original PEgRNA before swapping to the second template. Such an approach could enable long insertions by both preventing misfolding of the PEgRNA upon addition of the long template and also by not requiring dissociation of Cas9 from the genome for long insertions to occur, which could possibly be inhibiting PE-based long insertions.

In still other embodiments, the PEgRNA may be improved by introducing additional RNA motifs at the 5′ and 3′ termini of the PEgRNAs, or even at positions therein between (e.g., in the gRNA core region, or the spacer). Several such motifs—such as the PAN ENE from KSHV and the ENE from MALAT1 were discussed above as possible means to terminate expression of longer PEgRNAs from non-pol III promoters. These elements form RNA triple helices that engulf the polyA tail, resulting in their being retained within the nucleus^{184, 187}. However, by forming complex structures at the 3′ terminus of the PEgRNA that occlude the terminal nucleotide, these structures would also likely help prevent exonuclease-mediated degradation of PEgRNAs.

Other structural elements inserted at the 3′ terminus could also enhance RNA stability, albeit without enabling termination from non-pol III promoters. Such motifs could include hairpins or RNA quadruplexes that would occlude the 3′ terminus¹⁹⁷, or self-cleaving ribozymes such as HDV that would result in the formation of a 2′-3′-cyclic phosphate at the 3′ terminus and also potentially render the PEgRNA less likely to be degraded by exonucleases¹⁹⁸. Inducing the PEgRNA to cyclize via incomplete splicing—to form a ciRNA—could also increase PEgRNA stability and result in the PEgRNA being retained within the nucleus¹⁹⁴.

Additional RNA motifs could also improve RT processivity or enhance PEgRNA activity by enhancing RT binding to the DNA-RNA duplex. Addition of the native sequence bound by the RT in its cognate retroviral genome could enhance RT activity¹⁹⁹. This could include the native primer binding site (PBS), polypurine tract (PPT), or kissing loops involved in retroviral genome dimerization and initiation of transcription¹⁹⁹.

Addition of dimerization motifs—such as kissing loops or a GNRA tetraloop/tetraloop receptor pair²⁰⁰—at the 5′ and 3′ termini of the PEgRNA could also result in effective circularization of the PEgRNA, improving stability. Additionally, it is envisioned that addition of these motifs could enable the physical separation of the PEgRNA spacer and primer binding site, preventing occlusion of the spacer which would hinder PE activity. Short 5′ extensions or 3′ extensions to the PEgRNA that form a small toehold hairpin in the spacer region or along the primer binding site could also compete favorably against the annealing of intracomplementary regions along the length of the PEgRNA, e.g., the interaction between the spacer and the primer binding site that can occur. Finally, kissing loops could also be used to recruit other template RNAs to the genomic site and enable swapping of RT activity from one RNA to the other. A number secondary RNA structures that may be engineered into any region of the PEgRNA, including in the terminal portions of the extension arm (i.e., eland e2), as shown. Example improvements include, but are not limited to:

PEgRNA-HDV fusion
(SEQ ID) NO: 199)
GGCCCAGACTGAGCACGTGAGTTTTAGAGCTAGAAATAGCAAGTT
AAAATAAGGCTAGTCCGTTATCAACTTGAAAAAGTGGGACCGAGT
CGGTCCTCTGCCATCAAAGCGTGCTCAGTCTGGGCCGGCATGGTC
CCAGCCTCCTCGCTGGCGCCGGCTGGGCAACATGCTTCGGCATGG
CGAATGGGACTTTTTTT
PEgRNA-MMLV kissing loop
(SEQ ID NO: 200)
GGTGGGAGACGTCCCACCGGCCCAGACTGAGCACGTGAGTTTTAG
AGCTAGAAATAGCAAGTTAAAATAAGGCTAGTCCGTTATCAACTT
GAAAAAGTGGGACCGAGTCGGTCCTCTGCCATCAAAGCTTCGACC
GTGCTCAGTCTGGTGGGAGACGTCCCACCTTTTTTT
PEgRNA-VS ribozyme kissing loop
(SEQ ID NO: 201)
GAGCAGCATGGCGTCGCTGCTCACGGCCCAGACTGAGCACGTGAG
TTTTAGAGCTAGAAATAGCAAGTTAAAATAAGGCTAGTCCGTTAT
CAACTTGAAAAAGTGGGACCGAGTCGGTCCTCTGCCATCAAAGCT
TCGACCGTGCTCAGTCTCCATCAGTTGACACCCTGAGGTTTTTTT
PEgRNA-GNRA tetraloop/tetraloop receptor
(SEQ ID NO: 202)
GCAGACCTAAGTGGUGACATATGGTCTGGGCCCAGACTGAGCACG
TGAGTTTTAGAGCTAUACGTAGCAAGTTAAAATAAGGCTAGTCCG
TTATCAACTTUACGAAGTGGGACCGAGTCGGTCCTCTGCCATCAA
AGCTTCGACCGTGCTCAGTCTGCATGCGATTAGAAATAATCGCAT
GTTTTTTT
PEgRNA template switching secondary RNA-
HDV fusion
(SEQ ID NO: 203)
TCTGCCATCAAAGCTGCGACCGTGCTCAGTCTGGTGGGAGACGTC
CCACCGGCCGGCATGGTCCCAGCCTCCTCGCTGGCGCCGGCTGGG
CAACATGCTTCGGCATGGCGAATGGGACTTTTTTT

PEgRNA scaffolds could be further improved via directed evolution, in an analogous fashion to how SpCas9 and prime editors (PE) have been improved. Directed evolution could enhance PEgRNA recognition by Cas9 or evolved Cas9 variants. Additionally, it is likely that different PEgRNA scaffold sequences would be optimal at different genomic loci, either enhancing PE activity at the site in question, reducing off-target activities, or both. Finally, evolution of PEgRNA scaffolds to which other RNA motifs have been added would almost certainly improve the activity of the fused PEgRNA relative to the unevolved, fusion RNA. For instance, evolution of allosteric ribozymes composed of c-di-GMP-I aptamers and hammerhead ribozymes led to dramatically improved activity²⁰², suggesting that evolution would improve the activity of hammerhead-PEgRNA fusions as well. In addition, while Cas9 currently does not generally tolerate 5′ extension of the sgRNA, directed evolution will likely generate enabling mutations that mitigate this intolerance, allowing additional RNA motifs to be utilized. The present disclosure contemplates any such ways to further improve the efficacy of the prime editing systems utilized in the methods and compositions disclosed here.

In various embodiments, it may be advantageous to limit the appearance of consecutive sequence of Ts from the extension arm as consecutive series of T's may limit the capacity of the PEgRNA to be transcribed. For example, strings of at least consecutive three T's, at least consecutive four T's, at least consecutive five T's, at least consecutive six T's, at least consecutive seven T's, at least consecutive eight T's, at least consecutive nine T's, at least consecutive ten T's, at least consecutive eleven T's, at least consecutive twelve T's, at least consecutive thirteen T's, at least consecutive fourteen T's, or at least consecutive fifteen T's should be avoided when designing the PEgRNA, or should be at least removed from the final designed sequence. In one embodiment, one can avoid the includes of unwanted strings of consecutive T's in PEgRNA extension arms but avoiding target sites that are rich in consecutive A:T nucleobase pairs.

### PEgRNAs for Evading MMR

The present disclosure also provides novel pegRNAs for use in prime editing. As discussed above, prime editing using pegRNAs having DNA synthesis templates that comprise three or more consecutive nucleotide mismatches relative to a target site sequence can evade correction by the MMR pathway, resulting in an increase in prime editing efficiency and/or a decrease in the frequency of indel formation compared to the introduction of a single nucleotide mismatch using prime editing. Thus, the present disclosure provides pegRNAs useful for introducing modifications into a target nucleic acid with increased prime editing efficiency and/or decreased indel frequency compared to a corresponding control pegRNA that does not contain three or more consecutive nucleotide mismatches relative to the target site sequence.

The pegRNAs provided by the present disclosure are useful for editing a nucleic acid molecule by prime editing while improving prime editing efficiency and/or reducing indel formation. Without wishing to be bound by theory, the pegRNAs provided in the present disclosure may evade or reduce the impact of cellular MMR correction of mismatches at the target site that are introduced by the nucleotide alteration(s) through prime editing. In some embodiments, the extension arm of the pegRNAs provided by the present disclosure comprise three or more consecutive nucleotide mismatches relative to a target site on the nucleic acid molecule. In some embodiments, the DNA synthesis template of the pegRNA comprises three or more consecutive nucleotide mismatches relative to the target site on the nucleic acid molecule. In some embodiments, at least one of the three or more consecutive nucleotide mismatches introduces a silent mutation. In some embodiments, at least one of the consecutive nucleotide mismatches results in an alteration in the amino acid sequence of a protein expressed from the target nucleic acid molecule, while at least one of the remaining nucleotide mismatches is a silent mutation. The silent mutations may be introduced in coding regions of the target nucleic acid molecule or in non-coding regions of the target nucleic acid molecule. When the silent mutations are introduced in a coding region, they may introduce into the nucleic acid molecule one or more alternate codons encoding the same amino acid as the unedited nucleic acid molecule. When the silent mutations are introduced in a non-coding region, they may be present in a region of the nucleic acid molecule that does not influence splicing, gene regulation, RNA lifetime, or other biological properties of the target site on the nucleic acid molecule.

Any number of consecutive nucleotide mismatches of three or more can be incorporated into the extension arm of the pegRNAs described herein to achieve the benefits of avoiding or reducing the impact of correction by the cellular MMR pathway. In some embodiments, the DNA synthesis template of the extension arm on the pegRNA comprises three or more, four or more, five or more, six or more, seven or more, eight or more, nine or more, or ten or more consecutive nucleotide mismatches relative to a target site on the nucleic acid molecule.

Any number of consecutive nucleotide mismatches of three or more can be incorporated into the extension arm of the pegRNAs described herein to achieve the benefits of evading correction by the MMR pathway, thereby increasing editing efficiency and/or reducing unintended indels. In some embodiments, the DNA synthesis template of the extension arm on the pegRNA comprises three consecutive nucleotide mismatches relative to the endogenous sequence of a target site on the nucleic acid molecule edited by prime editing. In some embodiments, the DNA synthesis template of the extension arm on the pegRNA comprises 3-5 consecutive nucleotide mismatches relative to the endogenous sequence of a target site on the nucleic acid molecule edited by prime editing. In some embodiments, the DNA synthesis template of the extension arm on the pegRNA comprises 6-10 consecutive nucleotide mismatches relative to the endogenous sequence of a target site on the nucleic acid molecule edited by prime editing. In some embodiments, the DNA synthesis template of the extension arm on the pegRNA comprises 11-20 consecutive nucleotide mismatches relative to the endogenous sequence of a target site on the nucleic acid molecule edited by prime editing. In some embodiments, the DNA synthesis template of the extension arm on the pegRNA comprises 20-25 consecutive nucleotide mismatches relative to the endogenous sequence of a target site on the nucleic acid molecule edited by prime editing. In some embodiments, the DNA synthesis template of the extension arm on the pegRNA comprises 3, 4, 5, 6, 7, 8, 9, 10, 11, 12, 13, 14, 15, 16, 17, 18, 19, 20, 21, 22, 23, 24, or 25 consecutive nucleotide mismatches relative to the endogenous sequence of a target site on the nucleic acid molecule edited by prime editing. In some embodiments, the DNA synthesis template of the extension arm on the pegRNA comprises three or more, four or more, five or more, six or more, seven or more, eight or more, nine or more, or ten or more consecutive nucleotide mismatches relative to the endogenous sequence of a target site on the nucleic acid molecule edited by prime editing.

### Kits

The compositions of the present disclosure may be assembled into kits. In some embodiments, the kit comprises nucleic acid vectors for the expression of a prime editor and an MMR inhibitor, such as, but not limited to an MLH1 dominant negative variant as described herein. In other embodiments, the kit further comprises appropriate guide nucleotide sequences (e.g., PEgRNAs and second-site gRNAs) or nucleic acid vectors for the expression of such guide nucleotide sequences, to target the Cas9 protein or prime editor to the desired target sequence.

The kit described herein may include one or more containers housing components for performing the methods described herein and optionally instructions for use. Any of the kit described herein may further comprise components needed for performing the assay methods. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe and shipped refrigerated. Alternatively, it may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink wrapped pouch, a vacuum scalable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the various components of the prime editing system utilized in the methods and compositions described herein (e.g., including, but not limited to, the napDNAbps, reverse transcriptases, polymerases, fusion proteins (e.g., comprising napDNAbps and reverse transcriptases (or more broadly, polymerases), extended guide RNAs, and complexes comprising fusion proteins and extended guide RNAs, as well as accessory elements, such as second strand nicking components (e.g., second strand nicking gRNA) and 5′ endogenous DNA flap removal endonucleases for helping to drive the prime editing process towards the edited product formation). In some embodiments, the nucleotide sequence(s) comprises a heterologous promoter (or more than a single promoter) that drives expression of the prime editing system components.

Other aspects of this disclosure provide kits comprising one or more nucleic acid constructs encoding the various components of the prime editing systems utilized in the methods and compositions described herein, e.g., the comprising a nucleotide sequence encoding the components of the prime editing system capable of modifying a target DNA sequence. In some embodiments, the nucleotide sequence comprises a heterologous promoter that drives expression of the prime editing system components.

Some aspects of this disclosure provides kits comprising a nucleic acid construct, comprising (a) a nucleotide sequence encoding a napDNAbp (e.g., a Cas9 domain) fused to a reverse transcriptase and (b) a heterologous promoter that drives expression of the sequence of (a).

### Cells

Cells that may contain any of the compositions described herein include prokaryotic cells and eukaryotic cells. The methods described herein are used to deliver a Cas9 protein or a prime editor and an MMR inhibitor (e.g., an MLH1 dominant negative variant) into a eukaryotic cell (e.g., a mammalian cell, such as a human cell). In some embodiments, the cell is in vitro (e.g., cultured cell. In some embodiments, the cell is in vivo (e.g., in a subject such as a human subject). In some embodiments, the cell is ex vivo (e.g., isolated from a subject and may be administered back to the same or a different subject).

Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells. SHSY5Y human neuroblastoma cells (cloned from a myeloma) and Saos-2 (bone cancer) cells. In some embodiments, rAAV vectors are delivered into human embryonic kidney (HEK) cells (e.g., HEK 293 or HEK 293T cells). In some embodiments. rAAV vectors are delivered into stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR 1, EMT6/AR 10.0, FM3, H1299, H69, HB54. HB55, HCA2, Hepalclc7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3, . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, TIHP1, U373, U87, U937, VCaP, WM39, WT-49, X63, YAC-1 and YAR cells.

Some aspects of this disclosure provide cells comprising any of the constructs disclosed herein. In some embodiments, a host cell is transiently or non-transiently transfected with one or more vectors described herein. In some embodiments, a cell is transfected ex vivo. In some embodiments, a cell is transfected in vivo. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A 10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293, BxPC3, C3H-10T1/2, C6136, Cal-27, CHO, CHO-7, CHO-IR, CHO-K1, CHO-K2, CHO-T, CHO Dhfr −/−, COR-L23, COR-L23/CPR, COR-L2315010, COR-L23/R23, COS-7, COV-434, CML T1, CMT, CT26, D17, DH82, DU145, DuCaP, EL4, EM2, EM3, EMT6/AR 1, EMT6/AR 10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see. e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

### Vectors

Some aspects of the present disclosure relate to using recombinant virus vectors (e.g., adeno-associated virus vectors, adenovirus vectors, or herpes simplex virus vectors) for the delivery of the prime editors and MLH1 dominant negative mutants as described herein into a cell. In the case of a split-PE approach, the N-terminal portion of a PE fusion protein and the C-terminal portion of a PE fusion are delivered by separate recombinant virus vectors (e.g., adeno-associated virus vectors, adenovirus vectors, or herpes simplex virus vectors) into the same cell, since the full-length Cas9 protein or prime editors exceeds the packaging limit of various virus vectors, e.g., rAAV (˜4.9 kb).

In some embodiments, the vectors used herein may encode the PE fusion proteins, or any of the components thereof (e.g., napDNAbp, linkers, or polymerases), or an MLH1 dominant negative mutant. In addition, the vectors used herein may encode the PEgRNAs, and/or the accessory gRNA for second strand nicking. The vectors may be capable of driving expression of one or more coding sequences in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the promoters that may be used in the prime editor vectors may be constitutive, inducible, or tissue-specific. In some embodiments, the promoters may be a constitutive promoters. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EFla) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EFla promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. In some embodiments, the tissue-specific promoter is exclusively or predominantly expressed in liver tissue. Non-limiting exemplary tissue-specific promoters include B29 promoter, CD14 promoter, CD43 promoter, CD45 promoter, CD68 promoter, desmin promoter, elastase-I promoter, endoglin promoter, fibronectin promoter, Flt-1 promoter, GFAP promoter, GPIIb promoter, ICAM-2 promoter, INF-β promoter, Mb promoter. Nphsl promoter, OG-2 promoter, SP-B promoter, SYN1 promoter, and WASP promoter.

In some embodiments, the prime editor and MLH1 dominant negative mutant vectors (e.g., including any vectors encoding the prime editor fusion protein and/or the PEgRNAs, and/or the accessory second strand nicking gRNAs) may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the prime editor vectors (e.g., including any vectors encoding the prime editor fusion protein and/or the PEgRNAs, and/or the accessory second strand nicking gRNAs) and MLH1 dominant negative mutant vector (e.g., any vector encoding an MLH1 dominant negative mutant as described herein) may comprise tissue-specific promoters to start expression only after it is delivered into a specific tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-s promoter, Mb promoter, Nphsl promoter, OG-2 promoter, SP-B promoter, SYN1 promoter, and WASP promoter.

In some embodiments, the nucleotide sequence encoding the PEgRNA (or any guide RNAs used in connection with prime editing) may be operably linked to at least one transcriptional or translational control sequence. In some embodiments, the nucleotide sequence encoding the guide RNA may be operably linked to at least one promoter. In some embodiments, the promoter may be recognized by RNA polymerase III (Pol III). Non-limiting examples of Pol III promoters include U6, H I and tRNA promoters. In some embodiments, the nucleotide sequence encoding the guide RNA may be operably linked to a mouse or human U6 promoter. In other embodiments, the nucleotide sequence encoding the guide RNA may be operably linked to a mouse or human HI promoter. In some embodiments, the nucleotide sequence encoding the guide RNA may be operably linked to a mouse or human tRNA promoter. In embodiments with more than one guide RNA, the promoters used to drive expression may be the same or different. In some embodiments, the nucleotide encoding the crRNA of the guide RNA and the nucleotide encoding the tracr RNA of the guide RNA may be provided on the same vector. In some embodiments, the nucleotide encoding the crRNA and the nucleotide encoding the tracr RNA may be driven by the same promoter. In some embodiments, the crRNA and tracr RNA may be transcribed into a single transcript. For example, the crRNA and tracr RNA may be processed from the single transcript to form a double-molecule guide RNA. Alternatively, the crRNA and tracr RNA may be transcribed into a single-molecule guide RNA.

In some embodiments, the nucleotide sequence encoding the guide RNA may be located on the same vector comprising the nucleotide sequence encoding the PE fusion protein. In some embodiments, expression of the guide RNA and of the PE fusion protein may be driven by their corresponding promoters. In some embodiments, expression of the guide RNA may be driven by the same promoter that drives expression of the PE fusion protein. In some embodiments, the guide RNA and the PE fusion protein transcript may be contained within a single transcript. For example, the guide RNA may be within an untranslated region (UTR) of the Cas9 protein transcript. In some embodiments, the guide RNA may be within the 5′ UTR of the PE fusion protein transcript. In other embodiments, the guide RNA may be within the 3′ UTR of the PE fusion protein transcript. In some embodiments, the intracellular half-life of the PE fusion protein transcript may be reduced by containing the guide RNA within its 3′ UTR and thereby shortening the length of its 3′ UTR. In additional embodiments, the guide RNA may be within an intron of the PE fusion protein transcript. In some embodiments, suitable splice sites may be added at the intron within which the guide RNA is located such that the guide RNA is properly spliced out of the transcript. In some embodiments, expression of the Cas9 protein and the guide RNA in close proximity on the same vector may facilitate more efficient formation of the CRISPR complex.

The vector system may comprise one vector, or two vectors, or three vectors, or four vectors, or five vector, or more. In some embodiments, the vector system may comprise one single vector, which encodes both the PE fusion protein, the PEgRNA, and an MLH1 dominant negative mutant. In other embodiments, the vector system may comprise two vectors, wherein one vector encodes the PE fusion protein and the PEgRNA, and the other encodes the MLH1 dominant negative mutant.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. Delivery methods

In some aspects, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a prime editor as described herein in combination with (and optionally complexed with) a guide sequence, as well as an inhibitor of the DNA mismatch repair pathway, are delivered to a cell. In any of the delivery methods described herein, an inhibitor of the DNA mismatch repair pathway can also be delivered along with the prime editor. In some embodiments, the inhibitor is MLH1dn as described further herein. In some embodiments, the inhibitor is encoded on the same vector as the prime editor. In certain embodiments, the inhibitor is fused to the prime editor. In some embodiments, the inhibitor is encoded on a second vector, which is delivered along with a vector encoding the prime editor. In some embodiments, the prime editor fusion protein and the inhibitor of the DNA mismatch repair pathway are delivered to a cell as proteins directly. In certain embodiments, the prime editor is fused to the inhibitor of the DNA mismatch repair pathway, and the fusion protein is delivered directly into a cell.

Exemplary delivery strategies include vector-based strategies, PE ribonucleoprotein complex delivery, and delivery of PE by mRNA methods. In some embodiments, the method of delivery provided comprises nucleofection, microinjection, biolistics, virosomes, liposomes, immunoliposomes, polycation or lipid:nucleic acid conjugates, naked DNA, artificial virions, and agent-enhanced uptake of DNA.

Exemplary methods of delivery of nucleic acids include lipofection, nucleofection, electoporation, stable genome integration (e.g., piggybac), microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 91/17424; WO 91/16024. Delivery may be to cells (e.g., in vitro or ex vivo administration) or target tissues (e.g., in vivo administration). Delivery may be achieved through the use of RNP complexes.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In other embodiments, the method of delivery and vector provided herein is an RNP complex. RNP delivery of fusion proteins markedly increases the DNA specificity of base editing. RNP delivery of fusion proteins leads to decoupling of on- and off-target DNA editing. RNP delivery ablates off-target editing at non-repetitive sites while maintaining on-target editing comparable to plasmid delivery, and greatly reduces off-target DNA editing even at the highly repetitive VEGFA site 2. See Rees, H. A. et al., Improving the DNA specificity and applicability of base editing through protein engineering and protein delivery, Nat. Commun. 8, 15790 (2017), U.S. Pat. No. 9,526,784, issued Dec. 27, 2016, and U.S. Pat. No. 9,737,604, issued Aug. 22, 2017, each of which is incorporated by reference herein.

Additional methods for the delivery of nucleic acids to cells are known to those skilled in the art. See, for example, US 2003/0087817, incorporated herein by reference.

Other aspects of the present disclosure provide methods of delivering the prime editor constructs into a cell to form a complete and functional prime editor within a cell. For example, in some embodiments, a cell is contacted with a composition described herein (e.g., compositions comprising nucleotide sequences encoding the split Cas9 or the split prime editor or AAV particles containing nucleic acid vectors comprising such nucleotide sequences). In some embodiments, the contacting results in the delivery of such nucleotide sequences into a cell, wherein the N-terminal portion of the Cas9 protein or the prime editor and the C-terminal portion of the Cas9 protein or the prime editor are expressed in the cell and are joined to form a complete Cas9 protein or a complete prime editor.

It should be appreciated that any rAAV particle, nucleic acid molecule or composition provided herein may be introduced into the cell in any suitable way, either stably or transiently. In some embodiments, the disclosed proteins may be transfected into the cell. In some embodiments, the cell may be transduced or transfected with a nucleic acid molecule. For example, a cell may be transduced (e.g., with a virus encoding a split protein), or transfected (e.g., with a plasmid encoding a split protein) with a nucleic acid molecule that encodes a split protein, or an rAAV particle containing a viral genome encoding one or more nucleic acid molecules. Such transduction may be a stable or transient transduction. In some embodiments, cells expressing a split protein or containing a split protein may be transduced or transfected with one or more guide RNA sequences, for example in delivery of a split Cas9 (e.g., nCas9) protein. In some embodiments, a plasmid expressing a split protein may be introduced into cells through electroporation, transient (e.g., lipofection) and stable genome integration (e.g., piggybac) and viral transduction or other methods known to those of skill in the art.

### EXAMPLES

### Example 1: Enhanced Prime Editing Systems Through Identification and Manipulation of Cellular Determinants of Editing Outcomes

### Introduction

The ability to manipulate the genome in a programmable manner has illuminated biology and shown promise in the clinical treatment of genetic diseases. Towards the goal of expanding the breadth of sequence changes that can be precisely installed in living cells, prime editing was developed, a gene editing approach that enables all types of targeted DNA base pair substitutions, small insertions, small deletions, and combinations thereof without requiring double-strand DNA breaks or donor DNA templates (Anzalone et al., 2020; Anzalone et al., 2019). Prime editing has been broadly applied to introduce genetic changes in flies (Bosch et al., 2021), rice and wheat (Lin et al., 2020), zebrafish (Petri et al., 2021), mouse embryos (Liu et al., 2020), post-natal mice (Liu et al., 2021), human stem cells (Sirin et al., 2020), and patient-derived organoids (Schene et al., 2020). Despite its versatility, prime editing efficiency can vary widely across different edit classes, target loci, and cell types (Anzalone et al., 2019). To maximize the utility of prime editing, cellular determinants of prime editing outcomes were identified, and the resulting insights were used to develop prime editing systems with improved efficiency and outcome purity.

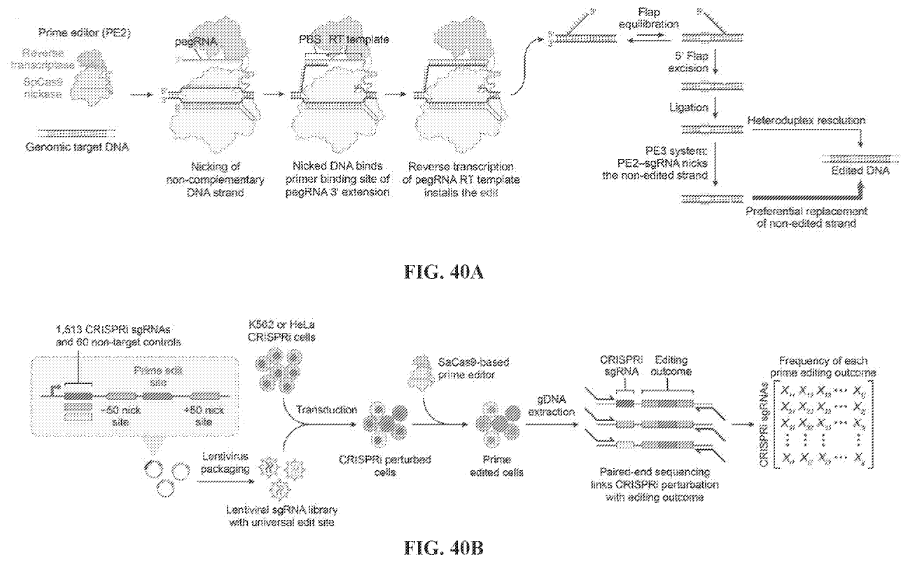

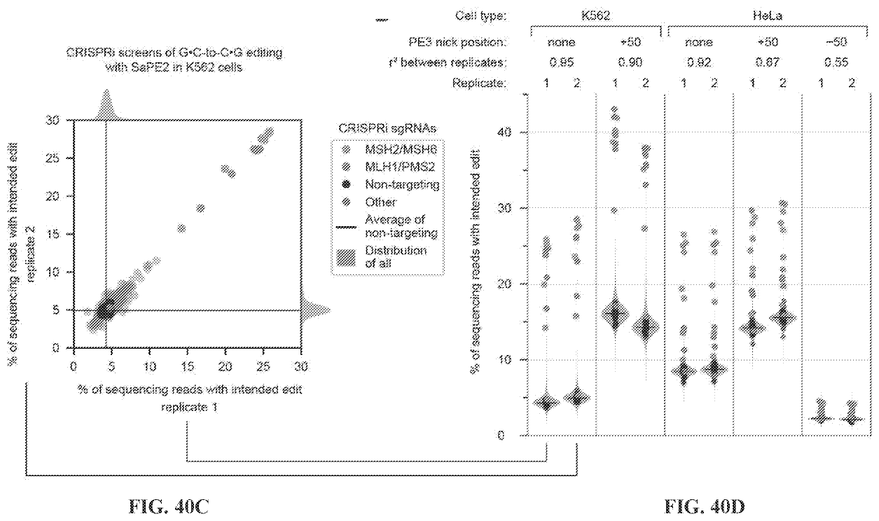

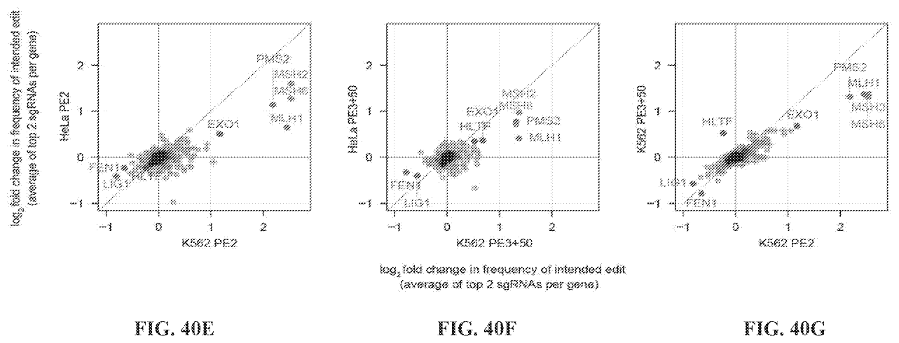

The PE2 prime editing system comprises two components: an engineered reverse transcriptase (RT) fused to a Cas9 nickase (the PE2 protein) and a prime editing guide RNA (pegRNA) that contains both a spacer sequence complementary to target DNA and a 3′ extension encoding the desired edit (Anzalone et al., 2020; Anzalone et al., 2019) (FIG. 40A). Together, these components form a PE2-pegRNA complex that binds one strand of a target DNA locus and nicks the opposite strand. This nick exposes a DNA 3′ end that can hybridize to the primer binding site (PBS) in the pegRNA extension. Reverse transcription of the RT template within the pegRNA extension then generates a 3′ DNA flap that contains the edited sequence and ultimately leads to installation of that sequence into the genome.

The PE3 system differs from PE2 by using an additional single guide RNA (sgRNA) to nick the non-edited strand away from the pegRNA target, which enhances editing efficiency by stimulating replacement of the non-edited strand (Anzalone et al., 2019) (FIG. 40A).

According to the current model, the newly synthesized 3′ flap displaces an adjacent strand of genomic DNA through flap interconversion (FIG. 40A). Excision of the displaced 5′ flap then allows ligation of the edited sequence into the genome. Nicking the non-edited strand in the PE3 system is thought to induce cellular replacement of the non-edited strand during heteroduplex resolution, and thus promotes copying of the edited sequence to the complementary strand.

The inventors studied the roles of DNA repair mechanisms in prime editing and the development of improved prime editing systems through manipulation of those processes. As presented herein, pooled CRISPR interference (CRISPRi)-based screens were used to systematically probe the effect of 476 genes involved in DNA repair and associated processes on substitution prime editing outcomes. Specific DNA mismatch repair (MMR) genes were identified that strongly suppress prime editing efficiency and promote indel formation. Consistent with a model in which MMR reverts heteroduplex DNA formed during prime editing, classes of prime edits were identified that are less vulnerable to MMR activity and are therefore generated more efficiently, including G⋅C-to-C⋅G transversions and substitutions of three or more contiguous bases. Integrating these findings, novel prime editing systems were developed with improved editing outcomes through transient expression of a dominant negative MMR protein (MLH1dn). In MMR-proficient cell types, including induced pluripotent stem cells and primary T cells, these PE4 (PE2+MLH1dn) and PE5 (PE3+MLH1dn) systems enhance editing efficiency over PE2 and PE3 by an average of 7.7-fold and 2.0-fold, respectively, and increase edit:indel ratios (outcome purity) by 3.4-fold. Transient co-expression of MLH1dn did not result in detected changes to microsatellite repeat length, a clinically used biomarker of MMR proficiency (Umar et al., 2004). By varying codon usage, nuclear localization signal, Cas9 domain mutations, and linker composition and length, an optimized prime editor architecture (PEmax—see FIG. 54B) was engineered that further increases editing efficiency in synergy with improvements from PE4, PE5, and recently developed engineered pegRNAs (epegRNAs) (Nelson et al., 2021). Finally, it was shown that strategic installation of additional silent mutations nearby an intended edit can improve prime editing efficiency by weakening MMR recognition. These findings deepen the understanding of prime editing and establish prime editing systems with substantially improved efficiency and outcome purity across 169 different edits at 19 loci in seven mammalian cell types.

### Results

### Design of a Pooled CRISPRi Screen for Prime Editing Outcomes

A new genetic screening approach called Repair-seq was used to study prime editing efficiency and outcome, including the original sequence, the desired edit, and indel frequency. Repair-seq measures the effects of many loss-of-function perturbations on the outcomes of genome editing experiments by linking the identity of CRISPRi sgRNAs to edited sites in pooled screens (Hussmann et al., 2021) (FIG. 40B). Briefly, a library of sgRNAs are lentivirally transduced into cells expressing the CRISPRi effector protein (dCas9-KRAB) such that most infected cells receive only one sgRNA, causing the knockdown of one gene per cell. A target sequence for genome editing is also delivered with the library sgRNAs in the same lentiviral cassette. After genome editing occurs at this target site, paired-end sequencing then enables the frequency of each editing outcome to be measured alongside each linked CRISPRi perturbation.

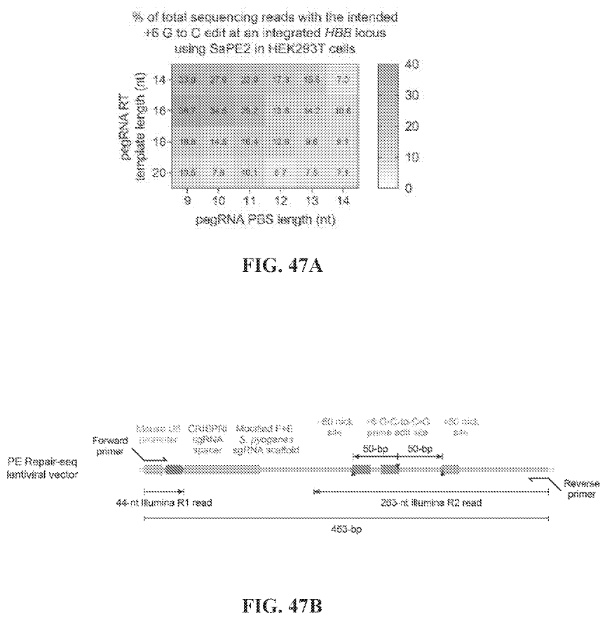

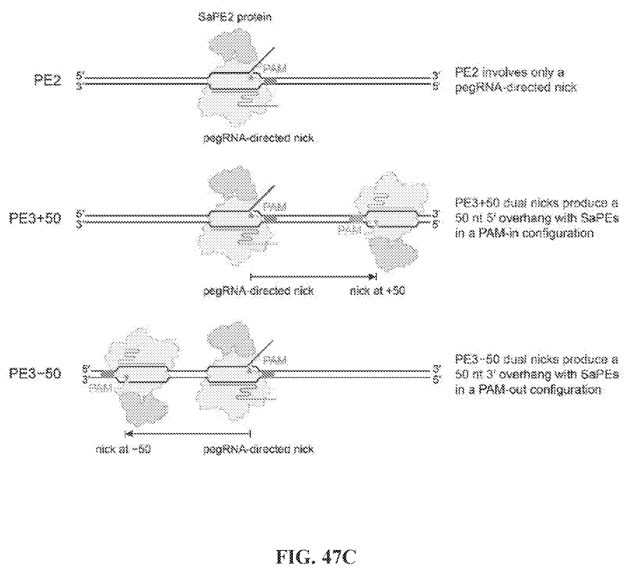

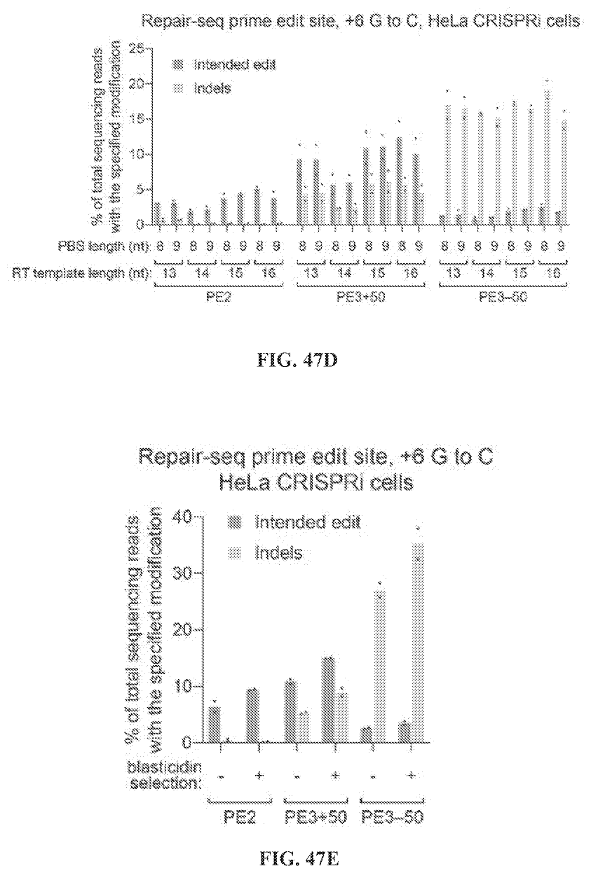

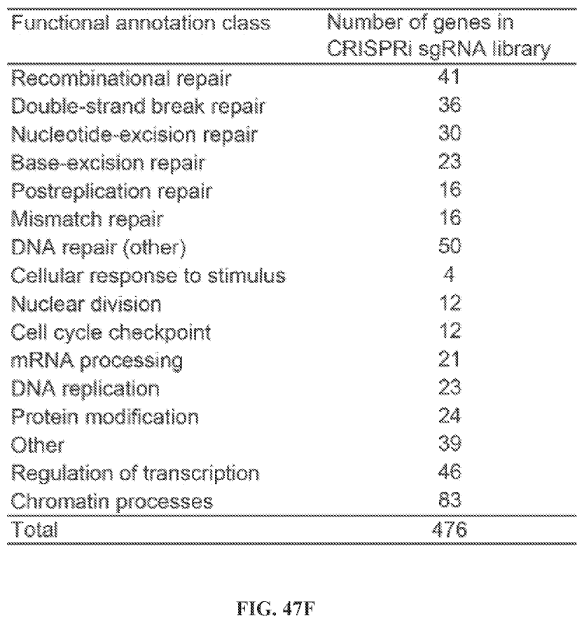

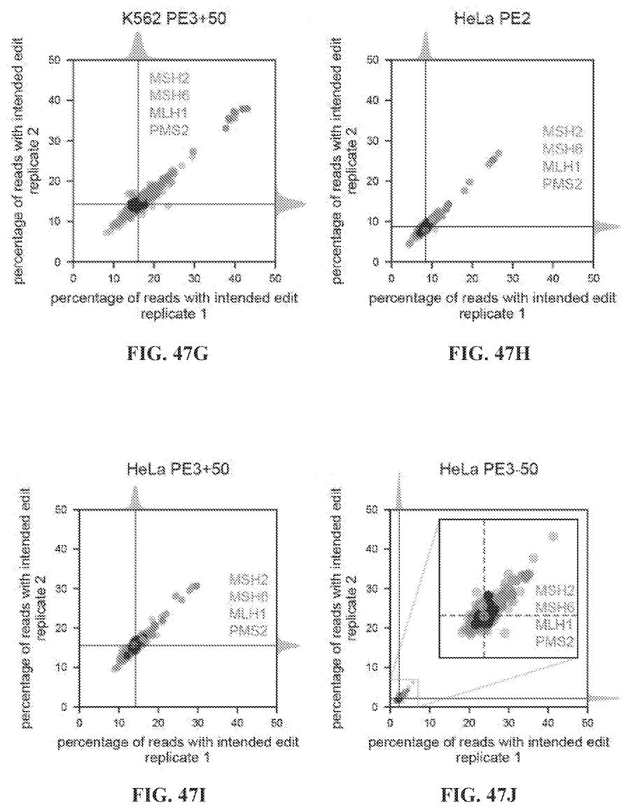

To adapt Repair-seq for prime editing, prime editing and CRISPRi, which typically also relies on Streptococcus pyogenes Cas9 (SpCas9), were first made orthogonal. An SaPE2 prime editor variant was constructed by replacing the SpCas9 nickase domain in PE2 with Staphylococcus aureus Cas9 (SaCas9) N580A nickase (Ran et al., 2015). Prime editing activity was verified using SaPE2 and orthogonal S. aureus pegRNAs (Sa-pegRNAs) (FIG. 47A).

Next, a lentiviral Repair-seq vector was designed for screening with SaPE2 by adding a composite SaPE2 edit site. This site was comprised of a single target protospacer that was identified to be efficiently edited in HEK293T cells (FIG. 47A) and two flanking protospacers that allowed complementary-strand (the non-edited strand) nicks 50-bp downstream (+50 nick) or upstream (−50 nick) of the target (FIG. 40B. FIG. 47B). This design supported prime editing with SaPE2 in three approaches: PE2, PE3 with a +50 nick (PE3+50), or PE3 with a −50 nick (PE3-50) (FIG. 47C).

To enable screening, Sa-pegRNAs were optimized for editing this site in a validated HeLa CRISPRi cell line (Gilbert et al., 2013). Transfection of these cells with SaPE2, Sa-pegRNA, and Sa-sgRNA plasmids that program a G⋅C-to-C⋅G transversion at the edit site yielded up to 5.2% editing and 0.17% indels (PE2), 12% editing and 5.8% indels (PE3+50), and 2.6% editing and 19% indels (PE3-50) (FIG. 47D). The high proportion of indels from PE3-50 is consistent with past reports that 3′ overhangs left by dual nicks lead to a higher frequency of insertions, as compared to 5′ overhangs (Bothmer et al., 2017). Intended editing frequencies were increased to 9.4% with PE2, 15% with PE3+50, and 3.5% with PE3-50 using a SaPE2 construct carrying a blasticidin selection marker to enrich successfully transfected cells (FIG. 47E). Together, these efforts established a screening assay with a baseline of prime editing outcomes in an efficiency range well-suited to detect increases or decreases in editing from CRISPRi perturbations.

Identification of DNA Repair Components that Affect Prime Editing Outcomes

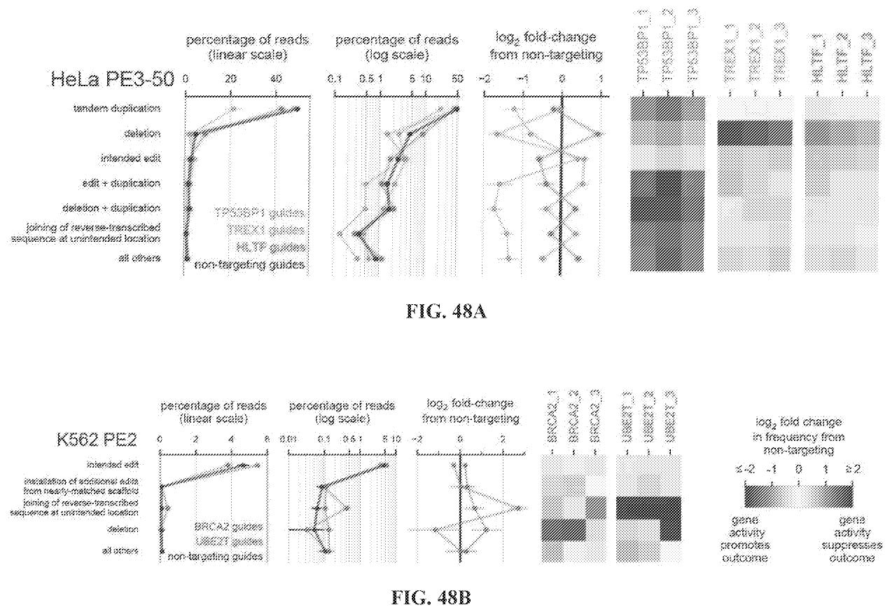

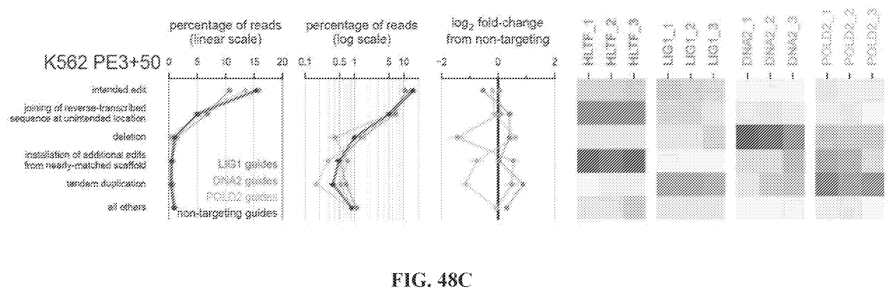

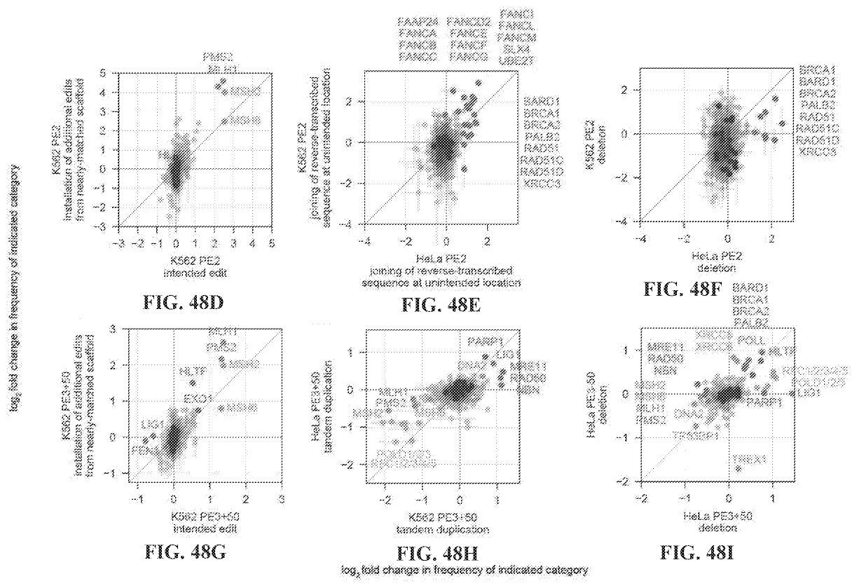

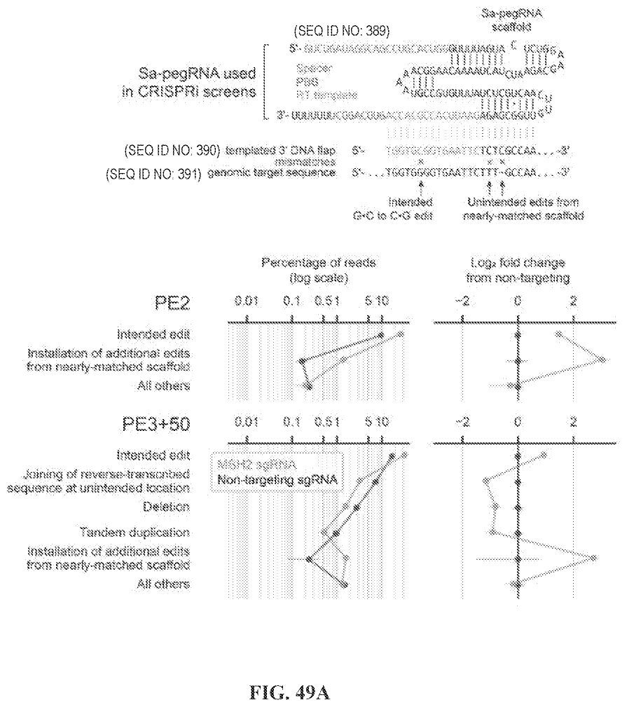

With this optimized assay, Repair-seq screens of prime editing outcomes were performed with PE2 and PE3+50 in K562 and HeLa cells and with PE3-50 in HeLa cells. A library of 1,513 sgRNAs targeting 476 genes enriched for roles in DNA repair and associated processes (FIG. 48F), along with 60 non-targeting control sgRNAs, were cloned into the lentiviral screening vector (Hussmann et al., 2021) (Table 5). This library was transduced into human K562 and HeLa CRISPRi cell lines (Gilbert et al., 2014: Gilbert et al., 2013) and, after 5 days, cells were transfected with SaPE2, Sa-pegRNA, and Sa-sgRNA plasmids that program a G⋅C-to-C⋅G transversion at the pre-validated edit site. Genomic DNA was extracted from cells 3 days after transfection, a 453-bp region containing the CRISPRi sgRNA, edit site, and complementary nick sites was amplified by PCR, and paired-end sequencing was performed to measure the distribution of editing outcomes for each genetic perturbation (FIG. 40B, FIG. 47B). To interpret the resulting data, the frequencies of editing outcomes from cells containing a gene-targeting CRISPRi sgRNA were compared to the corresponding frequencies from cells containing non-targeting sgRNA controls. Reduction in an outcome's frequency upon gene knockdown suggests that the gene's activity promotes formation of the outcome, while an increase in frequency suggests that the gene's activity suppresses the outcome.

The effect of gene knockdowns on the frequency of the intended G⋅C-to-C⋅G edit was examined. In cells with non-targeting CRISPRi sgRNAs, 4.3-4.9% (K562) and 8.5-8.7% (HeLa) of sequencing reads contained exactly the intended edit following PE2 editing (FIGS. 40C-40D). These levels increased to 14-16% (K562) and 14-16% (HeLa) for PE3+50, but decreased to 2.1-2.2% (HeLa) for PE3-50. Across all prime editing conditions screened, CRISPRi targeting of MSH2, MSH6, MLH1, and PMS2, components of the MutSα-MutLα MMR complex (Iyer et al., 2006; Kunkel and Erie, 2005; Li, 2008), substantially increased editing efficiency by up to 5.8-fold for PE2, 2.5-fold for PE3+50, and 2.0-fold for PE3-50 (FIGS. 40E-40G and 47G-47J, Table 6). Knockdown of EXO1, an exonuclease with a role in MMR (Genschel et al., 2002), also increased intended editing efficiency by up to 2.3-fold for PE2 in K562 cells. In contrast, knockdown of LIG1, a nick-sealing DNA ligase (Pascal et al., 2004), and of FEN1, a 5′ flap endonuclease (Liu et al., 2004), reduced the frequency of intended editing, consistent with their previously proposed roles in nick ligation and 5′ flap excision during prime editing (Anzalone et al., 2019). Together, these data suggest that MMR activity antagonizes the installation of point mutations by prime editing.

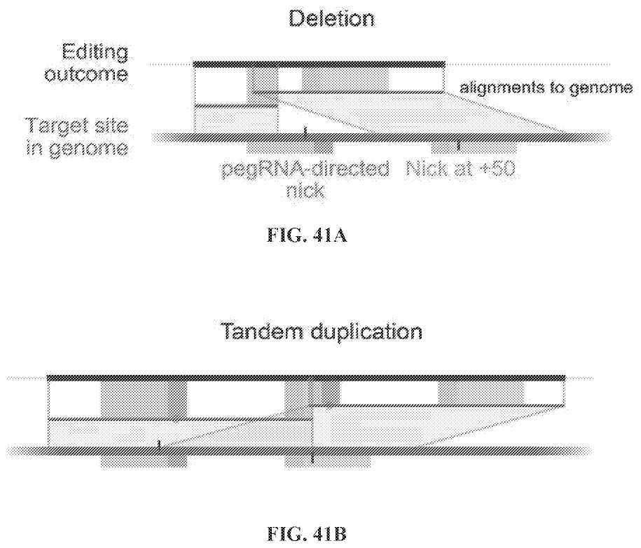

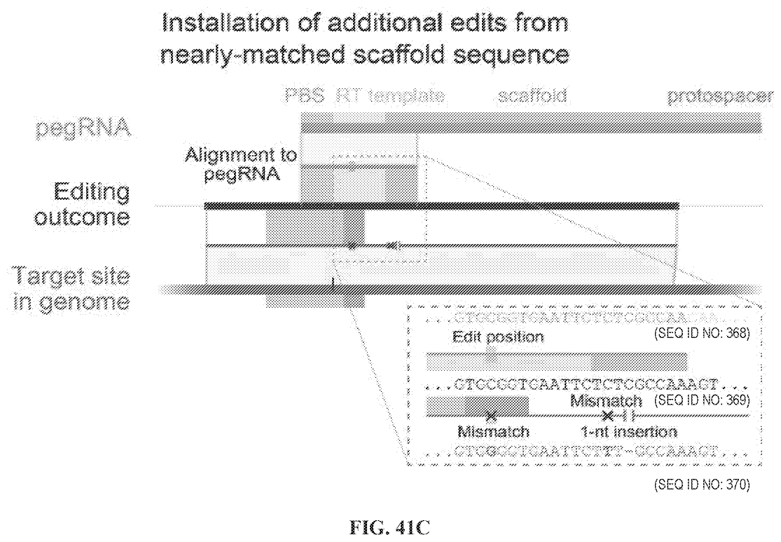

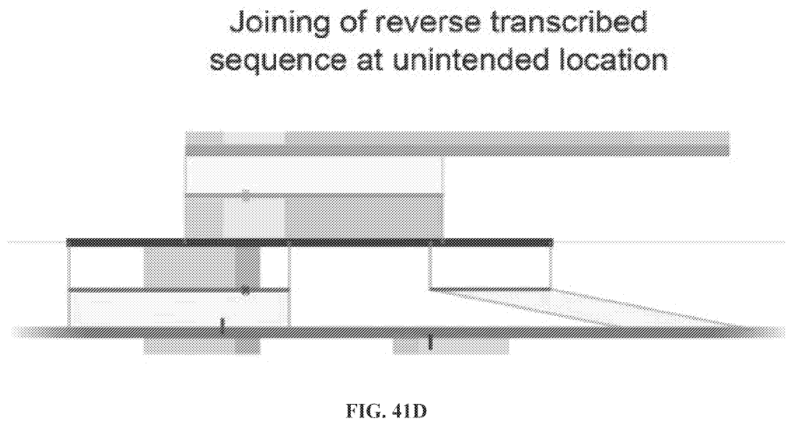

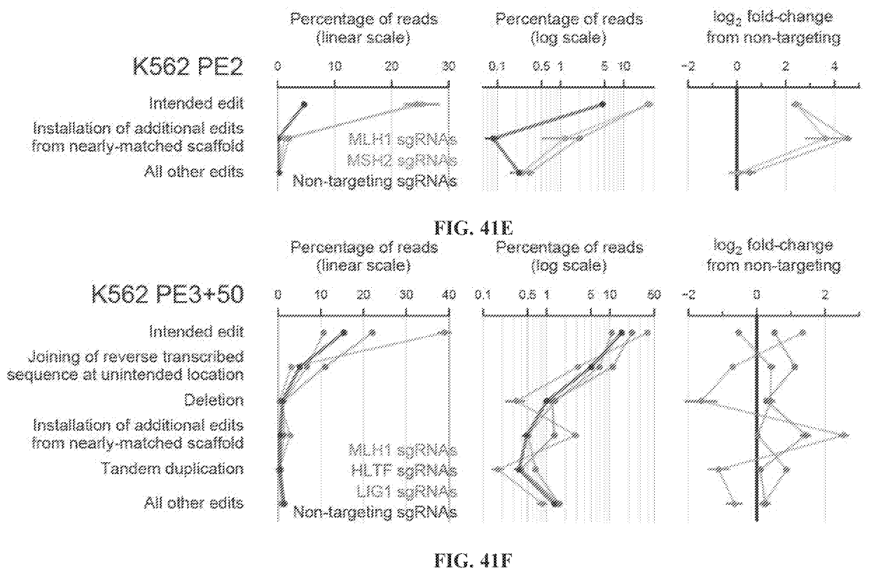

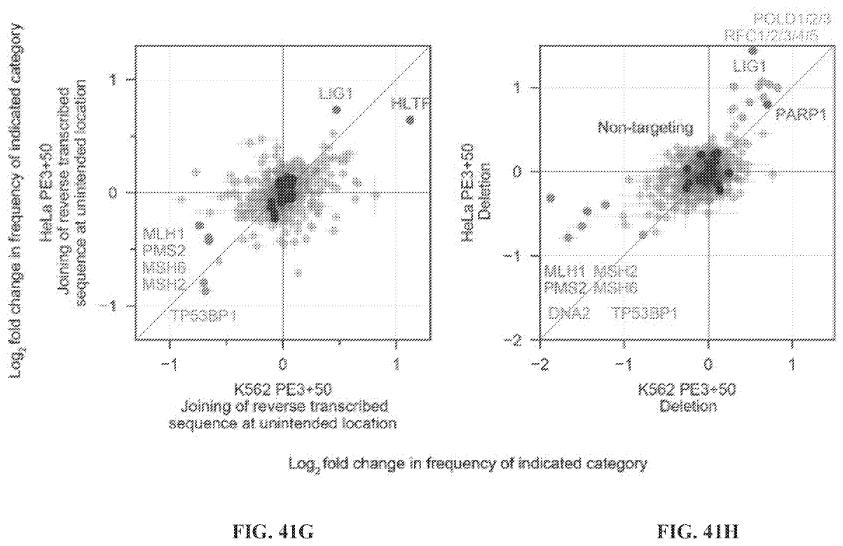

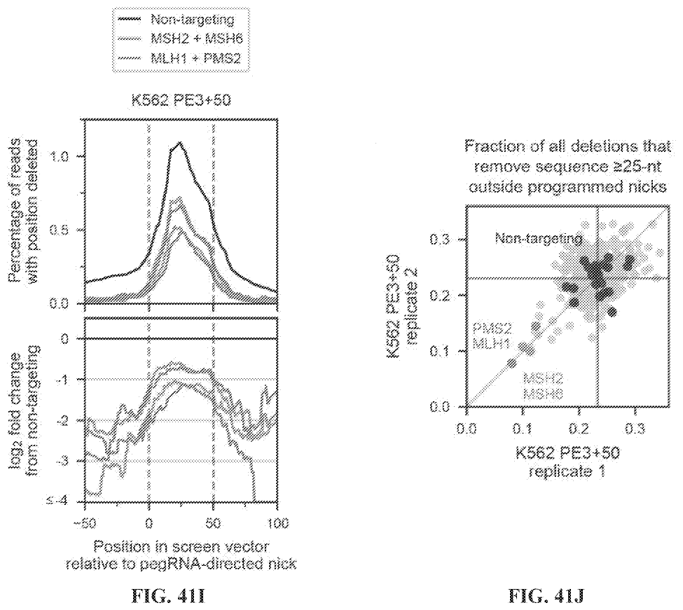

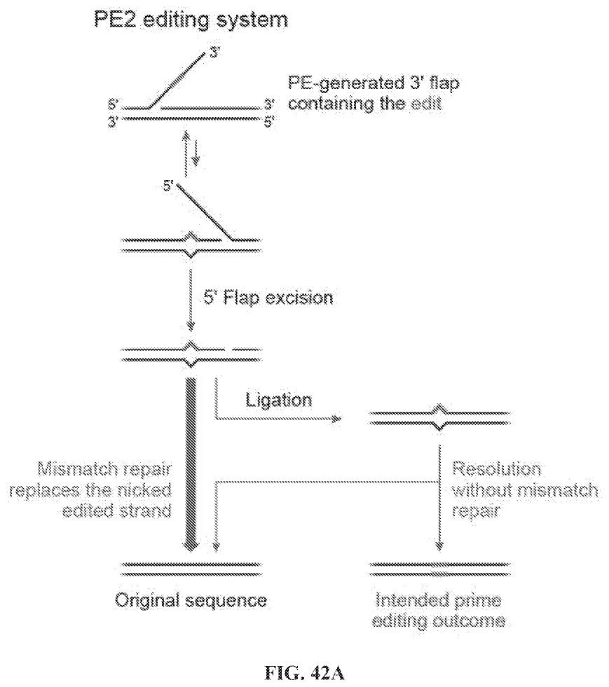

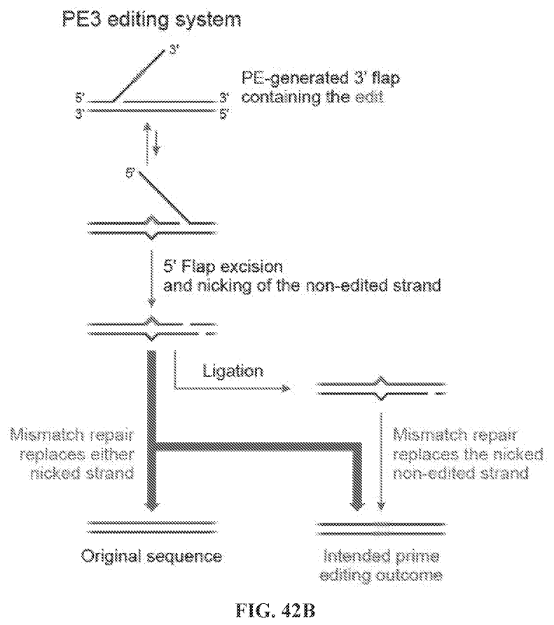

In addition to the intended edit, Repair-seq screens also measure the effects of genetic perturbations on the formation of editing byproducts. These byproducts were classified into four primary categories: deletions (FIG. 41A), tandem duplications (FIG. 41B), and two classes of outcomes containing unintended sequence from the pegRNA (FIGS. 41C-41D). Low baseline frequencies of total unintended edits from PE2 (0.31% in K562, 0.60% in HeLa; FIG. 41E), but more frequent and diverse unintended byproducts from PE3-50 (58% in HeLa; FIG. 48A) and PE3+50 (8.2% in K562, 9.5% in HeLa; FIG. 41F) were observed. The baseline frequencies and genetic modulators of these categories varied across PE2, PE3+50, and PE3-50 screens, providing a rich set of observations of how different prime editing configurations are processed (FIGS. 48A-48I). Two of these observations informed models for the role of MMR activity during prime editing.

First, one unintended outcome category contained the intended G⋅C to C⋅G edit as well as an additional base substitution and a 1-nucleotide (nt) insertion near the target site (FIG. 41C). The sequence at these additional mutations perfectly matched 9-nt at the 3′ end of the pegRNA scaffold sequence, consistent with reverse transcription into the pegRNA scaffold and incorporation of the resulting 3′ DNA flap into partially homologous genomic sequence. Recoding the pegRNA scaffold to avoid sequence homology with the genomic target strongly reduced the frequency of these additional mutations (FIGS. 49A-49B), suggesting a generalizable approach to eliminate this type of editing byproduct. Notably, it was observed that knockdown of MMR genes increased the frequency of this editing byproduct from 0.08% to 2.0% in PE2 K562 screens (FIG. 41E, FIG. 48D). MMR thus suppresses the formation of this outcome to a larger extent than the intended edit, demonstrating that distinct prime editing intermediates can differ in the extent to which they are processed by MMR.

Second, MMR knockdown reduced the frequency of most categories of unintended outcomes from PE3+50 (FIGS. 41F-41H and 48H), suggesting that transiently inhibiting some MMR activities may increase both the efficiency and outcome purity of prime editing. While tandem duplications of sequence between the nicks were common for PE3-50 (FIG. 48A), these duplication outcomes were rarer for PE3+50 (0.37% of non-targeting reads in K562, 2.3% in HeLa) and were reduced by up to 3.7-fold (K562) and 1.5-fold (HeLa) by MMR knockdown (FIG. 41F. FIG. 48H). The most abundant class of unintended PE3+50 outcomes contained sequence from the reverse-transcribed 3′ DNA flap that does not rejoin genomic sequence at the intended flap annealing location (5.1% of non-targeting reads in K562, 3.8% in HeLa; FIG. 41D). In both cell types, the frequency of unintended flap rejoining outcomes increased upon knockdown of HLTF, a fork remodeling helicase (Poole and Cortez, 2017), but was reduced by up to 1.7-fold by knockdown of MMR genes (FIG. 41H). Finally, MMR knockdown substantially reduced deletions from PE3+50 by up to 3.7-fold (K562) and 1.6-fold (HeLa; FIG. 41G). MMR knockdown in K562 cells also qualitatively shifted the observed boundaries of deleted sequence. For both PE3 configurations, genomic sequence between the two SaPE2-induced nicks was most frequently deleted, but deletions extending outside of this region were also observed (FIG. 41I, top). MMR knockdown decreased the frequency of these longer deletions dramatically more than deletions between the programmed nicks with PE3+50 (FIG. 41I, bottom and FIG. 41J), suggesting that MMR activity may cause the formation of longer deletions during prime editing. Model for mismatch repair of prime editing intermediates

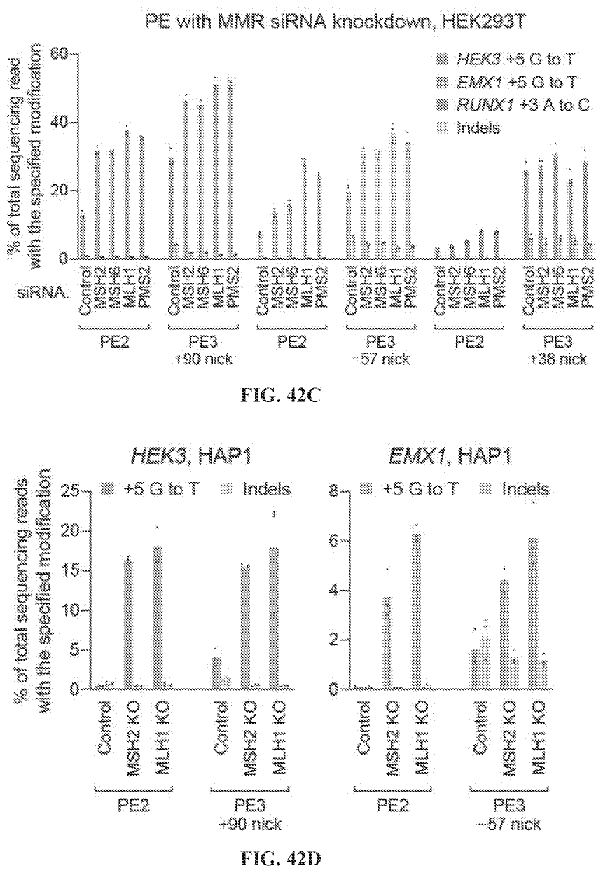

The effects of MMR knockdown on both intended and unintended editing outcomes in these Repair-seq screens led to a working model for the role of MMR during prime editing. In eukaryotes, MMR resolves DNA heteroduplexes containing a single base mismatch or small insertion-deletion loop (IDL) by selectively replacing the DNA strand that contains a nearby nick (Iyer et al., 2006; Kunkel and Erie, 2005; Li, 2008). To initiate MMR, the heteroduplex is first bound by MutSα (MSH2-MSH6), which recognizes base mismatches and 1- to 2-nt IDLs (Warren et al., 2007), or by MutSβ (MSH2-MSH3), which recognizes 2- to 13-nt IDLs (Gupta et al., 2012) (FIG. 42C). Next, MSH2 recruits the MutLα heterodimer (PMS2-MLH1), which incises only the nick-containing strand around the heteroduplex (Fang and Modrich, 1993; Kadyrov et al., 2006; Pluciennik et al., 2010: Thomas et al., 1991). From these incisions, EXO1 mediates 5′-to-3′ excision of the heteroduplex (Genschel et al., 2002), polymerase δ resynthesizes the excised DNA strand, and ligase I (LIG1) seals the nascent strand to complete repair (Iyer et al., 2006; Kunkel and Erie, 2005; Zhang et al., 2005).

Without wishing to be bound by theory, MMR may engage a specific prime editing intermediate: a DNA heteroduplex formed by hybridization of the reverse-transcribed 3′ DNA flap to the adjacent genomic DNA (FIG. 42A). If MutSα-MutLα recognizes the heteroduplex within this structure, the 3′ nick present after flap equilibration—but before ligation—could stimulate selective excision of the edited strand and subsequent repair to regenerate the original, unedited sequence. Alternatively, MMR may also prevent productive flap interconversion by rejecting annealing of the edited 3′ flap to the genomic target (Sugawara et al., 2004). In either case, inhibiting MMR during prime editing could delay heteroduplex repair or increase the likelihood of nick ligation. Moreover, successful nick ligation would remove the ability of MMR to bias resolution of the heteroduplex towards removal of the edited product. This model is supported by substantial increases in PE2 editing efficiencies (1.6- to 5.8-fold) from knockdown of MutSα-MutLα genes (FIG. 40C-40D), which strongly suggest that the 3′-nicked heteroduplex intermediate is often repaired by MMR to the original sequence before ligation of the nick (FIG. 42A). Interfering with MMR reversion of these intermediates can thus enhance prime editing efficiency.

This model also explains the benefit from complementary-strand nicks in the PE3 editing system. Nicking the non-edited strand of the heteroduplex intermediate introduces an additional strand discrimination signal that could direct MMR to more frequently replace the non-edited strand, leading to higher prime editing efficiency and dampened effects of MMR suppression (FIG. 42B). Consistent with this model, knockdown of MutSα-MutLα components increased intended G⋅C-to-C⋅G editing by up to 5.8-fold for PE2, but only by up to 2.6-fold for PE3+50 (FIG. 40G). MMR activity may also promote successful prime editing by directing excision of the non-edited strand, but only for a prime editing intermediate in which the edited strand has been ligated and the non-edited strand is nicked. However, MMR knockdown substantially enhances PE3+50 editing in K562 and HeLa cells (FIG. 40F), suggesting that this intermediate is uncommon and that MMR typically repairs the heteroduplex before nick ligation.

The mechanism of MMR may also explain how MutSα-MutLα gene knockdown reduces indel byproducts from PE3+50 (FIG. 41F). During repair of prime editing heteroduplex intermediates, MutLα may induce DSBs by indiscriminately nicking the target locus, particularly when both DNA strands already contain pegRNA- and sgRNA-programmed nicks (FIG. 49D). DSBs formed after excision from these additional, non-programmed nicks could broaden the boundaries of indels at prime edited loci. In agreement with this hypothesis, knockdown of MutSα-MutLα components disproportionately reduced PE3+50 deletion outcomes outside of the sequence between pegRNA and sgRNA nicks (FIG. 41I). Taken together, these findings from Repair-seq screens thus support a model in which MMR activity strongly suppresses intended prime editing outcomes and instead promotes indel byproducts.

### MMR Inhibition Improves Prime Editing at Endogenous Loci

The effect of MMR on prime editing was tested with canonical SpCas9-based prime editors at endogenous genomic loci and in additional cell types. HEK293T cells were treated with siRNAs targeting MSH2, MSH6, MLH1, or PMS2, the cells were cultured for 3 days to allow siRNA-mediated knockdown (FIG. 49E), then plasmids encoding PE2 and pegRNAs that program point mutations at three endogenous genomic loci were transfected (EMX1, RUNX1, and HEK293T cell site 3, hereafter referred to as HEK3). Across these loci, it was observed that mRNA knockdown strongly increased average PE2 editing from 7.7% to 25% with a decrease in indel frequency from 0.39% to 0.28% (FIG. 42C), but improved average PE3 editing efficiency to a lesser extent (from 25% to 37%). These results are consistent with the model presented herein, which states that complementary-strand nicking improves prime editing efficiency by directing MMR of the unedited strand. Thus, the impact of MMR on PE3 editing efficiency is tempered by its opposing effects on reverting the 3′ flap intermediate (which impedes prime editing) and on mediating replacement of the unedited strand (which promotes prime editing). Additionally, knockdown of MMR genes reduced the frequency of PE3 indels from 5.5% down to 3.2% on average, resulting in a 2.9-fold increase in PE3 outcome purity (FIG. 42C). These findings demonstrate that inhibition of MMR components can improve prime editing outcomes at endogenous genomic sites in human cells.

Prime editing was measured in MMR-deficient ΔMSH2 or ΔMLH1 haploid HAP1 cells. PE2 prime editing efficiency was much greater in MMR-deficient HAP1 cells (17% at HEK3, 5.0% at EMX1) than in wild-type control cells (0.44% at HEK3, 0.07% at EMX1; FIG. 42D). Moreover, consistent with the hypothesis stated herein, nicking the unedited strand at these loci did not affect editing efficiency in MMR-deficient HAP1 cells (FIG. 42D). Taken together, these results further support a model in which MMR impedes prime editing by promoting excision of the edited DNA strand, even though this effect is partially counterbalanced in the PE3 system by the role of MMR in replacing the non-edited strand.

### Engineered Dominant Negative MMR Proteins Enhance Prime Editing Efficiency and Precision

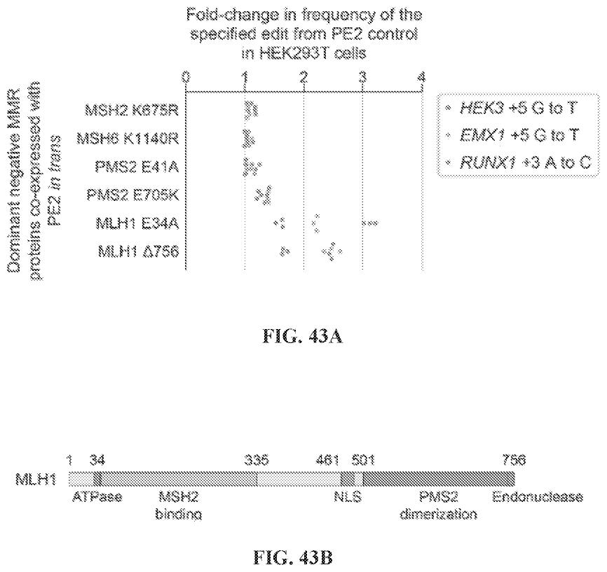

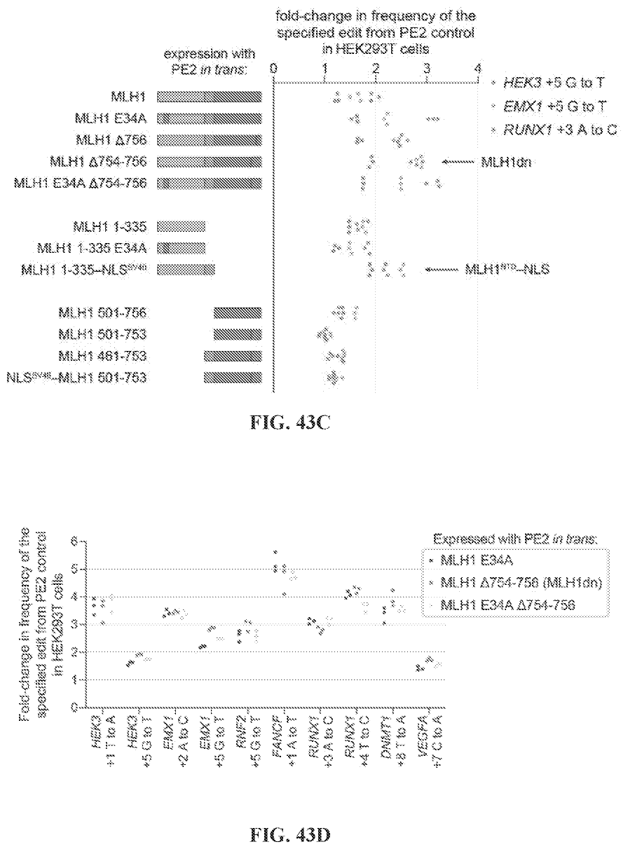

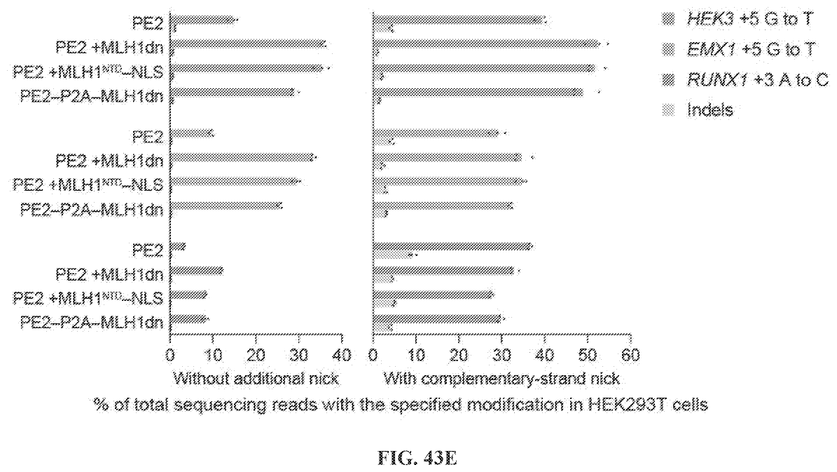

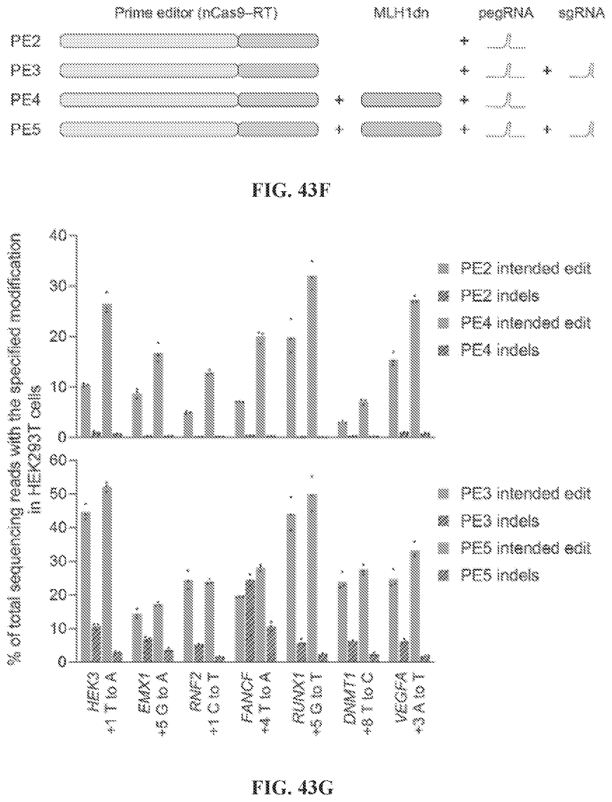

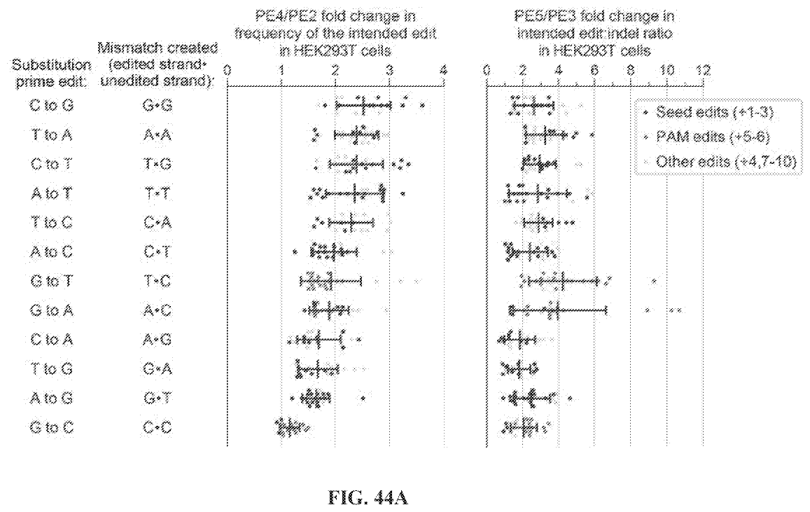

Encouraged that cellular pre-treatment with MMR-targeting siRNAs can enhance prime editing efficiency, strategies for simultaneous co-delivery of prime editors and MMR-inhibiting agents were explored. Co-transfection of PE2 and MLH1 siRNAs without pre-treatment did not substantially increase editing efficiency after 3 days (FIG. 49F). It was hypothesized that dominant negative MMR protein variants could instead be transiently co-expressed with PE2 or as fusion proteins with PE2 to enhance prime editing. HEK293T cells were co-transfected with plasmids encoding PE2, pegRNAs, and ATPase-deficient mutants of human MSH2, MSH6, PMS2, and MLH1 (laccarino et al., 1998; Räschle et al., 2002; Tomer et al., 2002), or endonuclease-deficient mutants of PMS2 and MLH1 (Gueneau et al., 2013; Kadyrov et al., 2006) (FIG. 43A). Of these mutants, ATPase-impaired MLH1 E34A and endonuclease-impaired MLH1 Δ756 increased PE2 editing efficiency by 1.6- to 3.1-fold for three single-base substitution edits at the HEK3, EMX1, and RUNX1 loci.

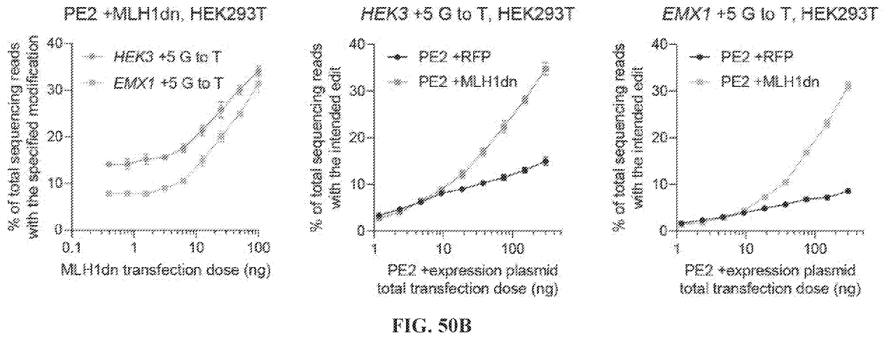

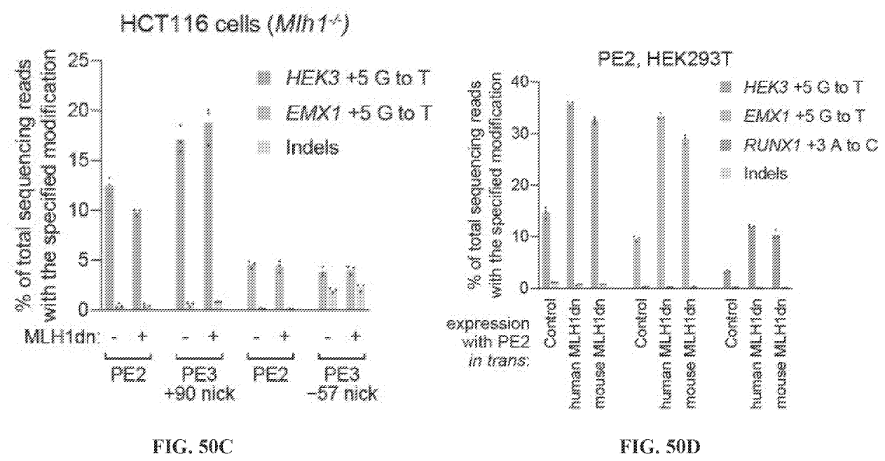

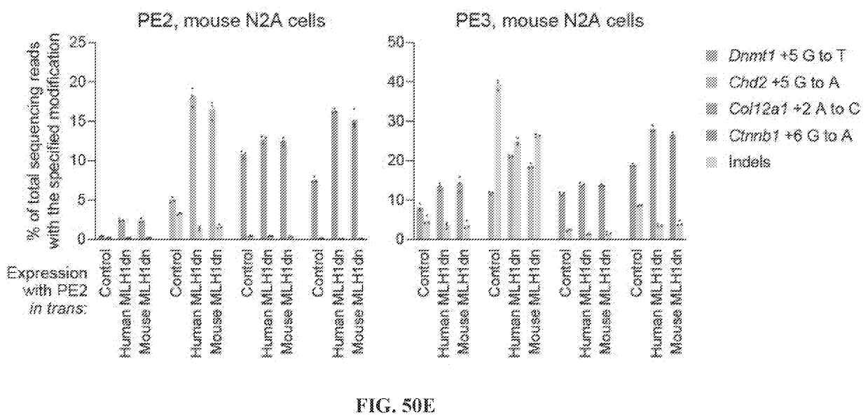

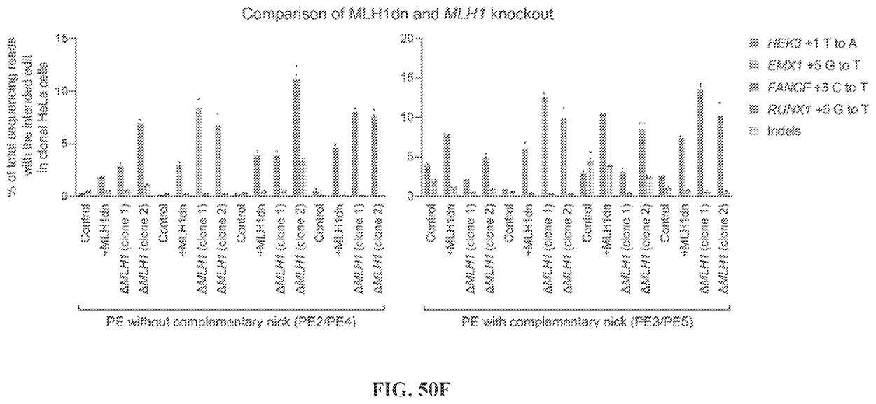

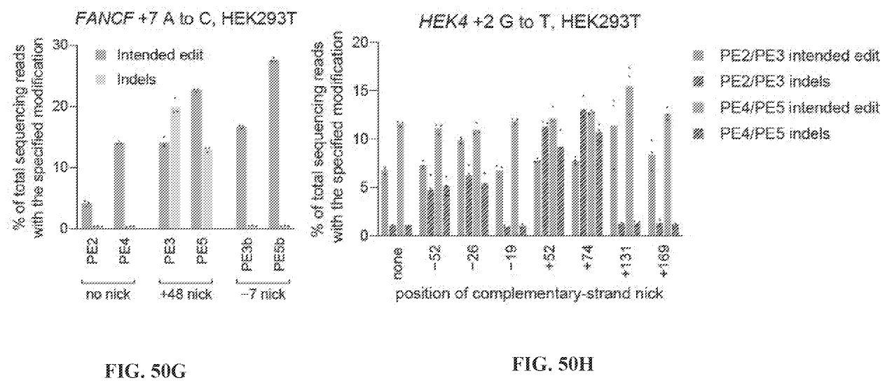

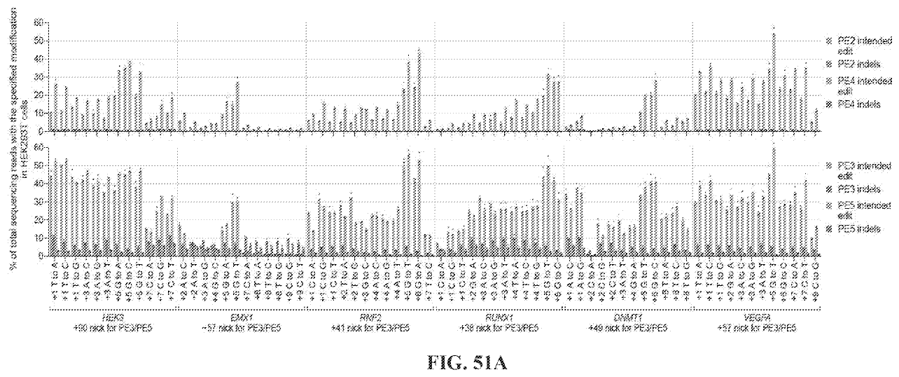

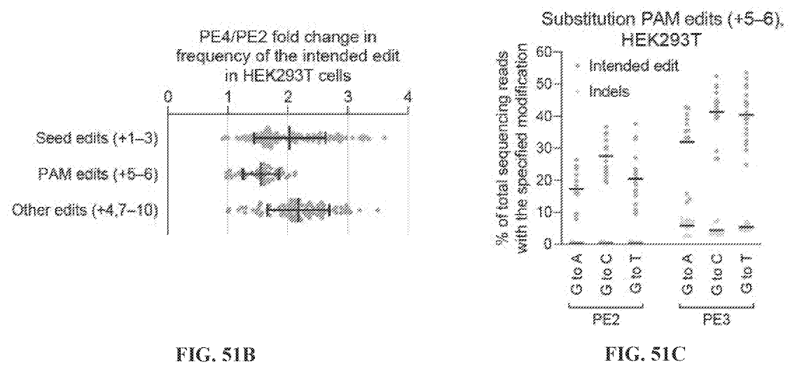

Next, additional dominant negative MLH1 variants were engineered and tested to maximize the enhancement of prime editing efficiency. The MLH1 N-terminal domain (NTD) mediates MutLα (PMS2-MLH1) recruitment to MSH2 during MMR (Plotz et al., 2003) and contains an ATPase essential for MutLα function (Kadyrov et al., 2006) (FIG. 43B). In contrast, the MLH1 C-terminal domain (CTD) dimerizes with PMS2 and contributes to MutLα endonuclease activity critical for MMR (Gueneau et al., 2013) (FIG. 43B). While the MLH1 Δ756 dominant negative variant disrupts this endonuclease, it was found that a larger deletion of these residues (MLH1 Δ754-756) further elevated prime editing efficiency at three sites tested (FIG. 43C, FIG. 50A). Combining ATPase and endonuclease mutations (ML H1 E34A Δ754-756) did not further improve prime editing, however (FIG. 43C). Comparing these dominant negative MLH1 variants across ten prime edits, it was observed that MLH1 Δ754-756 enhanced PE2 editing efficiencies to the greatest extent on average (by 3.2-fold: FIG. 50D). Thus, MLH1 Δ754-756 was designated as MLH1dn. MLH1dn improved prime editing in a dose-dependent manner within HEK293T cells (FIG. 50B). MLH1dn did not increase editing in MMR-deficient HCT116 cells (Parsons et al., 1993) (FIG. 50C). Both human and mouse MLH1dn also improved substitution prime editing efficiency across three sites in human HEK293T cells and four sites in mouse N2A cells (FIGS. 50D-50E).

Next, shorter MLH1 truncations that can also inhibit MMR during prime editing were identified. On its own, the MLH1 NTD (residues 1-335) modestly enhanced PE2 editing by 1.5- to 1.8-fold, but introducing the E34A ATPase mutation weakened this improvement at two of three tested loci (FIG. 43C, FIG. 50A). Appending a nuclear localization signal (NLS) to the MLH1 NTD boosted PE2 editing by 1.9- to 2.5-fold, to a similar degree as full-length MLH1dn. In contrast, the MLH1 CTD (residues 501-756), as well as endonuclease mutants and NLS-appended variants thereof, did not substantially enhance PE2 editing (FIG. 43C, FIG. 50A). These data suggest that dominant negative MLH1 variants can inhibit MMR by forming catalytically impaired MutLα complexes with PMS2 or by saturating the binding of MSH2, which would prevent productive recruitment of MutLα during MMR. Full-length MLH1dn, which can do both, could inhibit MMR through both mechanisms, but NLS-appended MLH1 NTD (hereafter referred to as MLH1^{NTD}-NLS) would be expected to inhibit MMR through MSH2 binding as it lacks the CTD needed to dimerize with PMS2 (Guerrette et al., 1999). MLH1^{NTD}-NLS improves PE2 editing efficiency to almost the same extent as MLH1dn (FIG. 43C, FIG. 50A), suggesting that MSH2 sequestration is an effective mechanism for inhibiting MMR.

MLH1dn or MLH1^{NTD}-NLS were also directly fused to the PE2 protein (via a 32aa (SGGS)x2-XTEN16-(SGGS)x2 linker), but did not exhibit higher editing efficiency compared to PE2 alone (FIG. 50A). However, appending MLH1dn to PE2 with a self-cleaving P2A linker (PE2-P2A-MLH1dn) (Kim et al., 2011) enhanced prime editing efficiency at three sites by 2.0- to 2.7-fold (FIG. 50A). Among the 55 total dominant negative MMR protein variants tested across three loci in HEK293T cells, PE2 and MLH1dn expressed in trans provided the greatest average enhancement in PE2 editing efficiency (3.2-fold). Strong average improvement of PE2 editing was also observed from PE2 with MLH1^{NTD}-NLS expressed in trans (2.7-fold) and PE2-P2A-MLH1dn (2.4-fold) (FIG. 43C). These three variants also increased PE3 editing efficiency by 1.2-fold on average but reduced undesired indel products by 1.4- to 4.0-fold (FIG. 43E). PE2 editing with MLH1dn was designated as the PE4 system, and PE3 editing with MLH1dn as the PE5 system (FIG. 43F). MLH1^{NTD}-NLS can also offer enhanced prime editing efficiencies with a smaller protein (355-aa) compared to MLH1dn (753-aa). When a single construct is required, the use of PE2-P2A-MLH1dn is recommended.

The generality of PE4 and PE5 systems were assessed with seven additional single-base substitution edits across different genomic loci in HEK293T cells. On average, PE4 improved editing efficiency over PE2 by 2.0-fold with minimal indels (<0.4% on average; FIG. 43G). PE5 improved editing over PE3 by 1.2-fold and enhanced edit:indel purity by 3.0-fold (FIG. 43G). Whether MLH H1dn could elevate the efficiency of PE3b, a prime editing strategy that uses a complementary-strand nick specific for the edited sequence to minimize coincident nicks on both strands that promote indel formation (Anzalone et al., 2019), was also tested. PE3b with co-expression of MLH1dn (hereafter referred to as PE5b) increased editing at the FANCF locus by 1.7-fold over PE3b with low indel frequencies (<0.6%; FIG. 50H). Moreover, it was shown that PE4 offers substantially improved prime editing performance compared to PE2 when indels must be minimized or at loci where complementary-strand nicks yield unproductive editing outcomes (FIG. 50H). Prime editing enhancement from MLH1dn versus complete MLH1 knockout was also compared in clonal HeLa cell lines. At four sites tested, MLH1 knockout enhanced PE2 and PE3 editing to a larger degree than MLH1dn co-expression (PE4 and PE5; FIG. 50F), suggesting opportunities for additional prime editing enhancement through further modulation of this pathway. Collectively, these data establish PE4 and PE5 systems that substantially enhance prime editing efficiency and outcome purity at a variety of endogenous genomic loci in human cells.

### Characterization of the Types of Prime Edits Enhanced by PE4 and PE5

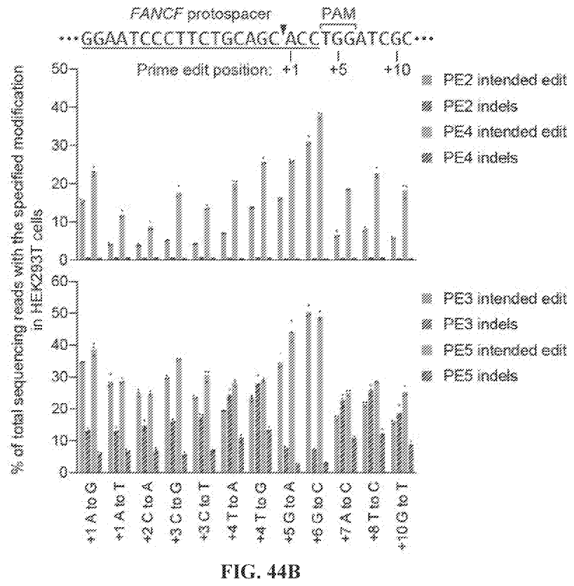

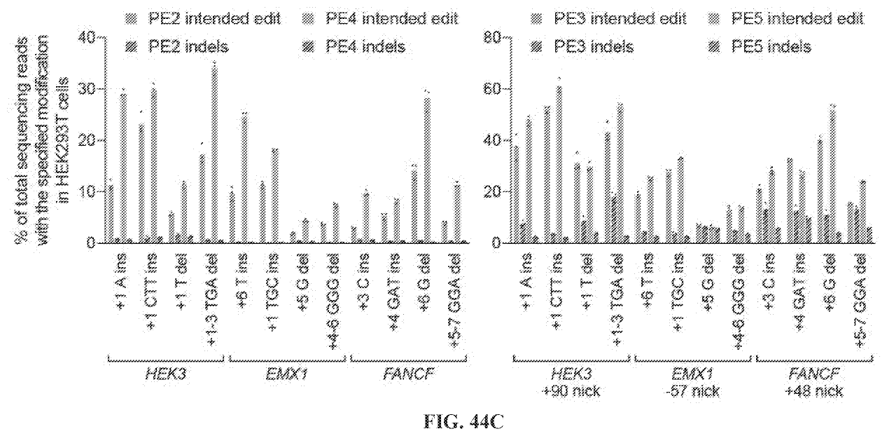

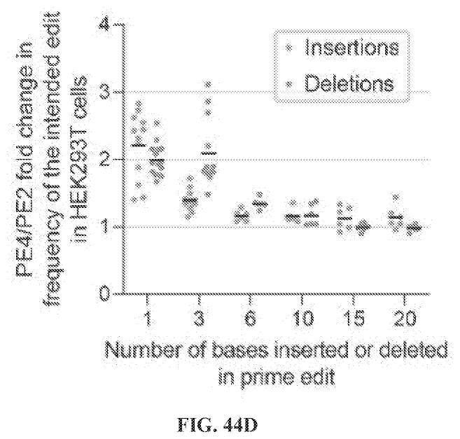

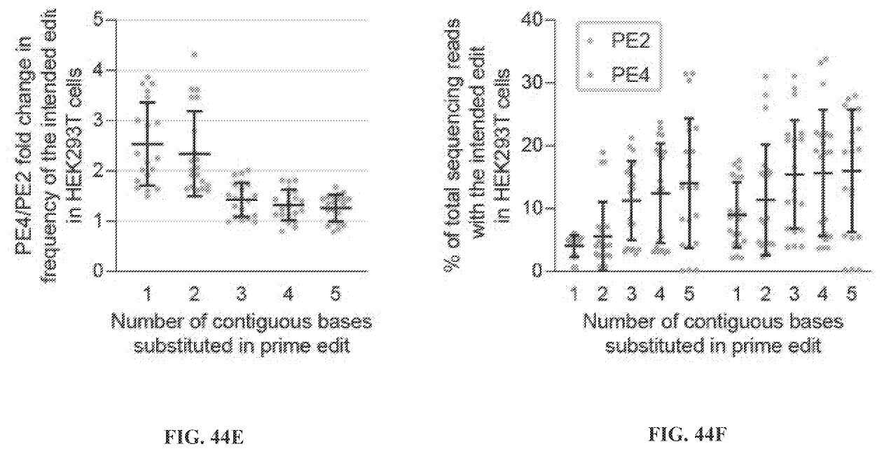

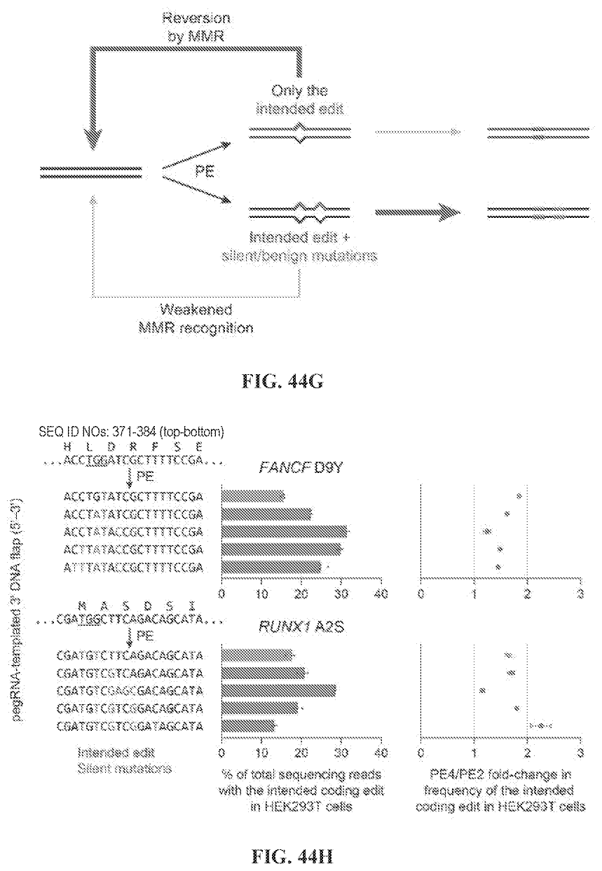

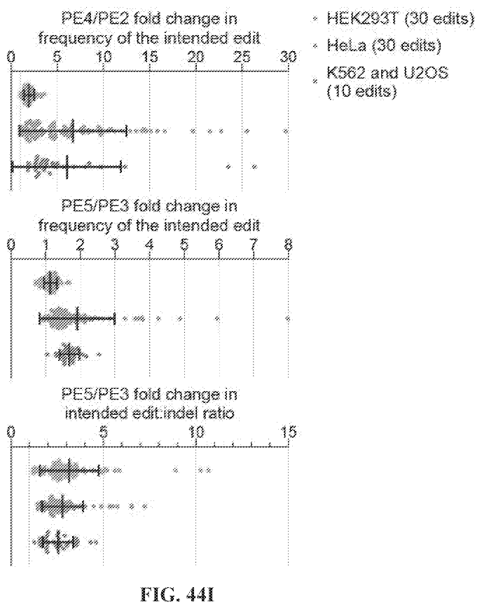

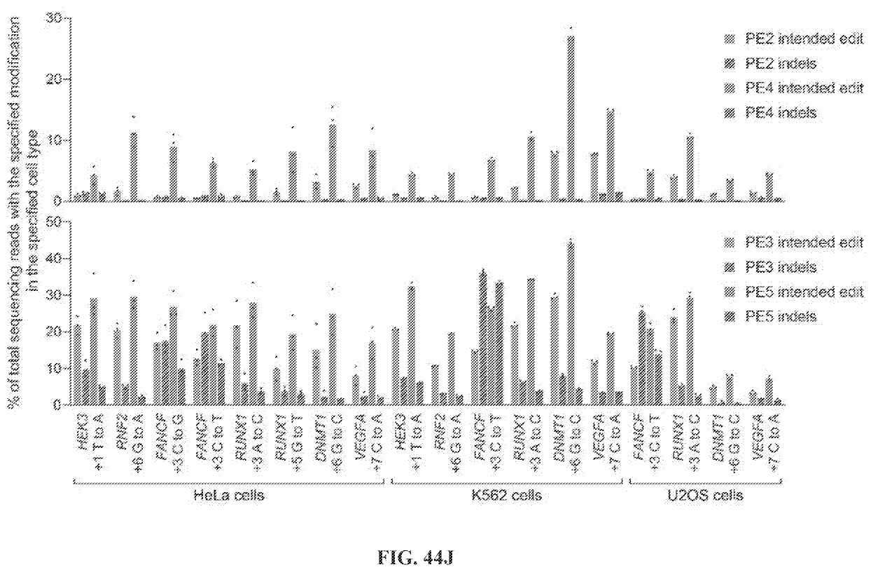

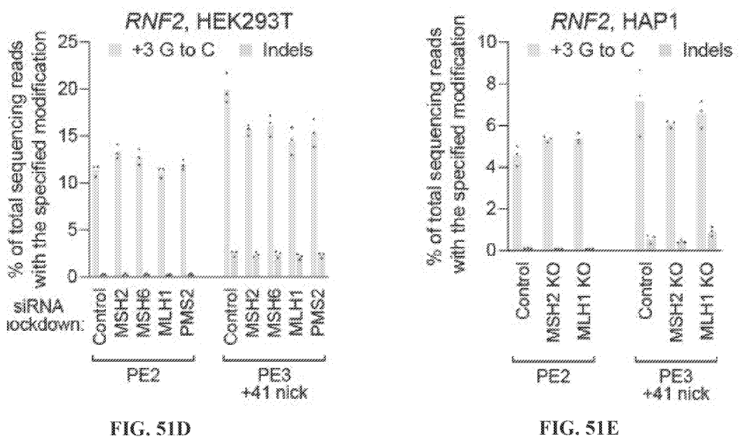

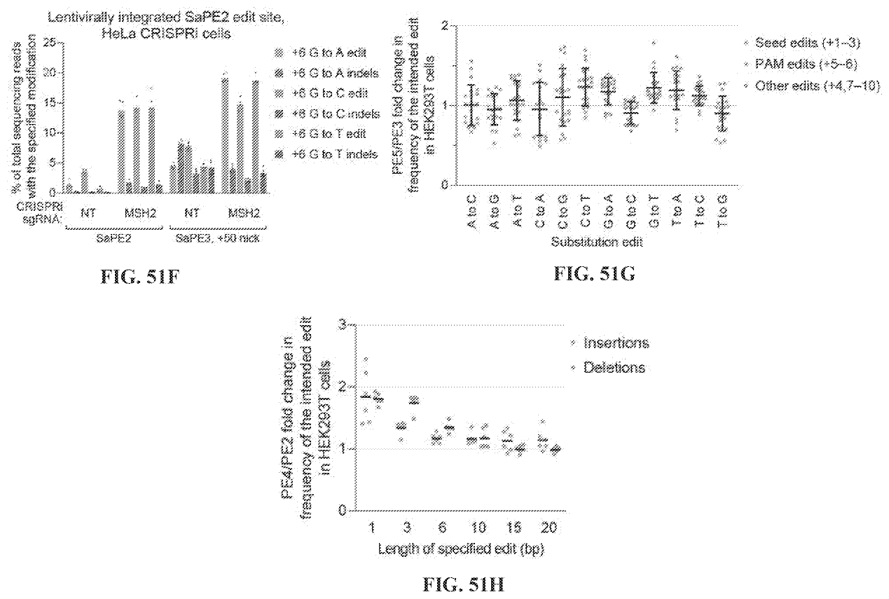

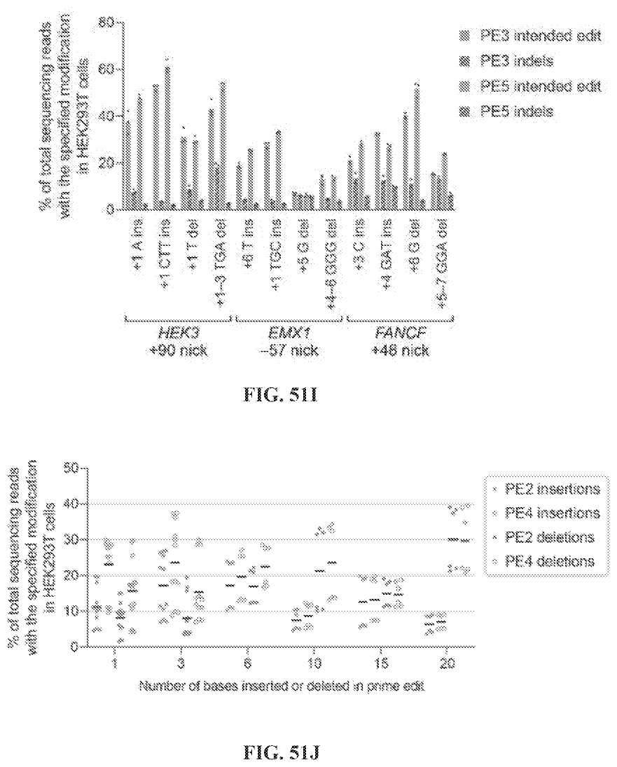

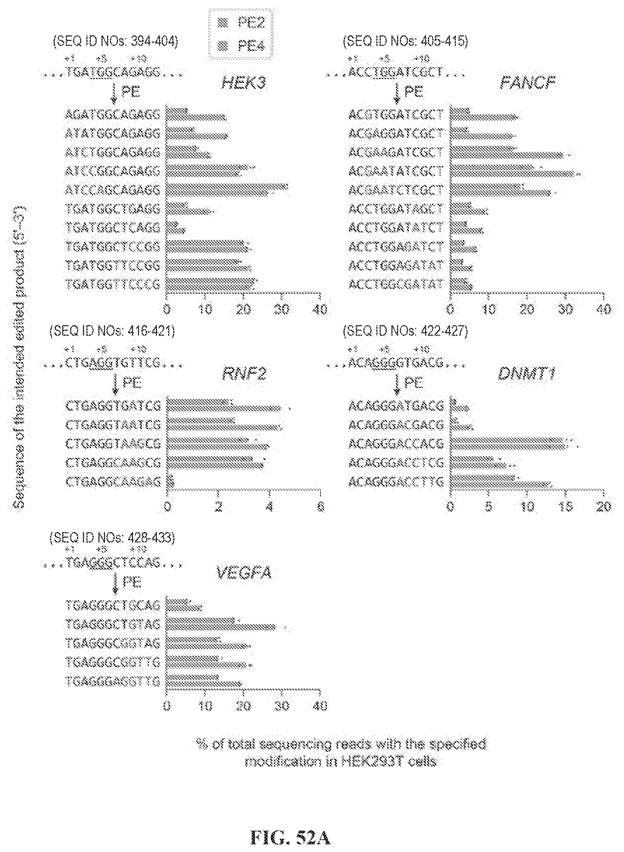

Next, the extent to which MLH1dn improves prime editing across a wide range of different edit types was studied. PE4 was compared with PE2 in HEK293T cells using 84 pegRNAs that together introduce all 12 possible single-base substitutions across seven genomic loci. Among these edits, MLH1dn improved prime editing efficiency by 2.0-fold and reduced average indel frequencies from 0.40% to 0.31% compared to PE2 (FIG. 44A-B, FIGS. 51A-51B).

MLH1dn increased the efficiency of A⋅T-to-G⋅C, T⋅A-to-G⋅C, and C⋅G-to-A⋅T prime edits to a lesser extent, by 1.7-fold over PE2. Notably, G⋅C-to-C⋅G substitutions, which form C⋅C mismatches after 3′ flap hybridization, were the least improved with MLH1dn (1.2-fold), consistent with previous studies establishing that C⋅C mismatches are not efficiently repaired by MMR (Lahue et al., 1989; Su et al., 1988; Thomas et al., 1991). This finding suggests that G⋅C-to-C⋅G edits more effectively evade MMR and may therefore yield higher basal editing efficiency. Consistent with this possibility, G⋅C-to-C⋅G edits with PE2 were substantially more efficient (27%) than G⋅C-to-A⋅T (18%) or G⋅C-to-T⋅A (20%) edits among PAM-altering prime edits across seven endogenous loci (FIG. 51C).

To further confirm that G⋅C-to-C⋅G substitutions are less sensitive to MMR, the effect of MMR knockdown or knockout was tested on the efficiency of a G⋅C-to-C⋅G prime edit at the RNF2 locus that is unaffected by MLH1dn co-expression (FIG. 51A). siRNA knockdown of MMR components in HEK293T cells (FIG. 51D) and knockout of MSH2 or MLH1 in HAP1 cells (FIG. 51E) did not change the efficiency of this G⋅C-to-C⋅G edit. G⋅C-to-A⋅T, G⋅C-to-C⋅G, and G⋅C-to-T⋅A edits were also compared with SaPE2 at the pre-validated screening site in HeLa CRISPRi cells (FIG. 40B). PE2 and PE3+50 more efficiently installed the G⋅C-to-C⋅G edit than the G⋅C-to-A⋅T or G⋅C-to-T⋅A edits, consistent with weaker MMR activity at C⋅C mismatches (FIG. 51F). Furthermore, CRISPRi knockdown of MSH2 improved G⋅C-to-A⋅T and G⋅C-to-T⋅A editing efficiencies (by 16-fold for PE2 and 4.3-fold for PE3+50) to a greater extent than G⋅C-to-C⋅G editing (by 4.0-fold for PE2 and 1.9-fold for PE3+50). These data strongly support that G⋅C-to-C⋅G prime edits are less susceptible to repair by MMR and are consequently installed with higher efficiency.

PE5 and PE3 were also compared with the same set of 84 pegRNAs used in the above experiments. PE5 yielded an average 1.2-fold increase in editing relative to PE3 (FIG. 44A, FIG. 51A, FIG. 51G). MLH1dn substantially reduced the frequency of unwanted indel products by 2.2-fold on average, resulting in 2.8-fold higher edit:indel purity (FIG. 44A). Collectively, this analysis of 84 different single-nucleotide substitutions at seven genomic loci strongly support a model in which PE4 and PE5 systems improve prime editing efficiency and outcome purity by impairing counterproductive MMR of prime editing intermediates.

To determine if MLH1dn could also improve small insertion and deletion prime edits, 1- and 3-bp insertions and deletions were next installed with PE4 and PE5 in HEK293T cells. Across 12 pegRNAs at three loci, it was observed that PE4 increased average editing efficiency by 2.2-fold over PE2 with no increase in indel frequency, while PE5 increased editing efficiency by 1.2-fold and edit:indel purity by 2.9-fold over PE3 (FIGS. 44C and 51I). To evaluate the impact of MLH1dn on larger sequence changes, PE2 and PE4 were tested with a combined 33 pegRNAs that together program 1-, 3-, 6-, 10-, 15-, and 20-bp insertions and deletions at the HEK3 and FANCF loci. It was found that the enhancement in prime editing efficiency from MLH1dn gradually declined as the length of the insertions and deletions increased (FIGS. 44D and 51H), such that 15- and 20-bp insertions and deletions were edited by PE4 only 1.1-fold more efficiently on average than by PE2. These observations are consistent with previous reports that MMR repairs IDLs <13-nt in length (Acharya et al., 1996; Genschel et al., 1998; Umar et al., 1994). These results together demonstrate that PE4 and PE5 strategies can enhance small (<15-bp) targeted insertions and deletions.

### Installing Additional Silent Mutations can Increase Prime Editing Efficiency by Evading MMR

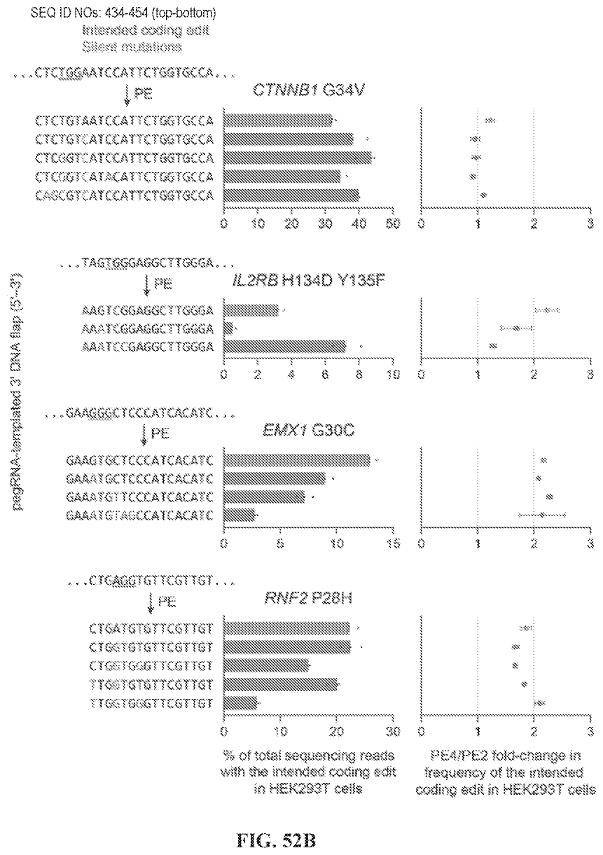

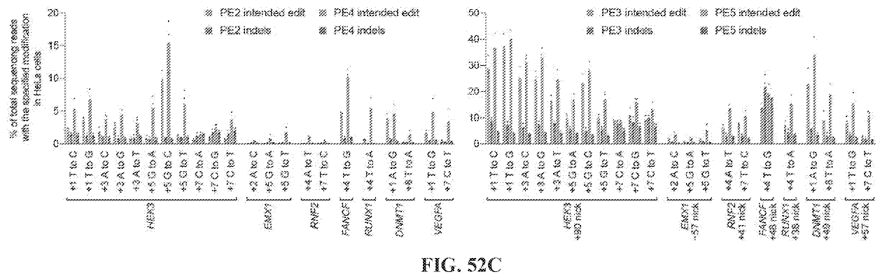

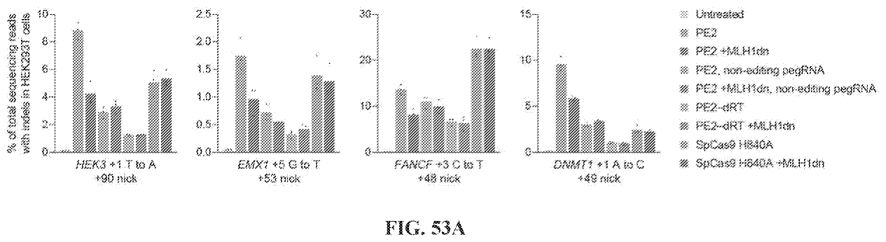

Whether other classes of prime edits could bypass MMR was also explored. In addition to point mutations, short insertions, and short deletions, prime editing can also install multiple neighboring or contiguous base substitutions (Anzalone et al., 2019). MutSα and MutSβ heterodimers each recognize specific heteroduplex DNA structures (Gupta et al., 2012; Warren et al., 2007), suggesting that a DNA bubble of contiguous mismatches could weaken recognition from these MMR components. To assess this possibility. PE2 and PE4 were tested with 35 different edits that generate 1-5 contiguous base substitutions at five genomic loci in HEK293T cells. Across seven edits that mutate two adjacent bases, PE4 yielded 2.3-fold higher editing efficiency than PE2, comparable to the 2.4-fold enhancement for single-base substitutions at the same target nucleotides (FIG. 44E, FIG. 52A). In contrast, the editing frequencies of longer 3- to 5-base contiguous substitutions were only improved by 1.2- to 1.5-fold with PE4 relative to PE2. The reduced impact of MMR on these larger edits was reflected in higher average PE2 editing efficiency of for 3- to 5-bp contiguous substitutions (13% across 21 edits) compared to 1-2 bp contiguous substitutions (4.8% across 14 edits) (FIG. 44F, FIG. 52A). Inhibiting MMR with MLH1dn (PE4) raised the average editing frequency of these 3-5-bp and 1-2-bp contiguous substitutions to 16% and 10%, respectively, lessening the difference between these edit types.

Installing additional silent mutations nearby the intended edits could similarly increase prime editing efficiency by weakening repair of the resulting heteroduplex (FIG. 51G), even in the absence of any MLH1 inhibition. PE2 was tested at six gene targets with either pegRNAs that program only a coding mutation or with pegRNAs that program additional silent mutations close to the coding edit (most fewer than 5 bp away). At four of six sites, making additional silent mutations increased the efficiency of the desired coding changes, by an average of 1.8-fold for the best pegRNAs at each site (FIGS. 44H and 52B). Inhibiting MMR with MLH1dn (PE4) improved editing efficiency with the best silent pegRNAs to a lesser extent (1.2-fold on average) than with pegRNAs that only make a coding mutation (1.7-fold), suggesting that these additional silent mutations enhance editing by evading MMR. Consistent with this mechanism, at the two sites in which silent pegRNAs do not improve editing, it was observed that the tested silent mutations do not substantially dampen the effect of MMR inhibition on editing efficiency (FIG. 52B). Collectively, these findings support that MMR less efficiently repairs heteroduplexes containing three or more contiguous mismatched bases and reveal, surprisingly, that the strategic installation of additional silent or benign mutations nearby the desired edit can increase prime editing efficiency by evading MMR reversal of prime editing intermediates, even when using PE2- or PE3-based systems that do not manipulate MMR activity.

PE4 and PE5 Strongly Improves Prime Editing in Cell Types with Fully Active MMR

HEK293T cells are partially MMR-deficient due to MLH1 promoter hypermethylation (Trojan et al., 2002), which in light of the above findings may explain higher prime editing efficiency observed in HEK293T cells compared to other mammalian cell types (Anzalone et al., 2019). To evaluate whether MLH1dn also improves prime editing to a greater degree in cells with fully active MMR, prime editing was compared in HEK293T cells and in MMR-proficient HeLa cells using 30 pegRNAs that together target seven loci. Consistent with higher MMR activity in HeLa cells (Holmes et al., 1990; Thomas et al., 1991), complementary-strand nicking (PE3 vs. PE2) resulted in much larger improvements in prime editing efficiency in HeLa cells (22-fold on average) than in HEK293T cells (3.5-fold; FIGS. 44I-44J and 52C.). Likewise, PE4 enhanced average editing efficiency over PE2 to a much greater extent in MMR-proficient HeLa cells (6.7-fold) than in partially MMR-deficient HEK293T cells (2.0-fold) across the same 30 edits, while maintaining minimal indel frequencies (0.67% on average in HeLa cells). Similarly, PE5 improved average editing efficiency over PE3 by 1.9-fold in HeLa cells but only by 1.1-fold in HEK293T cells. MLH1dn also increased edit:indel ratios on average by 2.8-fold in HeLa cells and 3.2-fold in HEK293T cells (FIG. 44I).

PE4 and PE5 were also assessed in MMR-proficient K562 and U2OS cell lines (Matheson and Hall, 2003; Peng et al., 2014). Across six sites in K562 cells and four sites in U2OS cells, PE4 and PE5 elevated average prime editing efficiency by 6.0-fold and 1.7-fold over PE2 and PE3, respectively (FIGS. 44I-44J), again offering a much larger benefit than was observed in MMR-impaired HEK293T cells. PE5 in these cells also improved edit:indel purity over PE3 by 2.6-fold. Intriguingly, although PE4 only increased G⋅C-to-C⋅G editing frequency at DNMT1 by 1.4-fold over PE2 in HEK293T cells (FIG. 51A), a larger improvement was observed in HeLa, K562, and U2OS cells (averaging 2.7-fold; FIG. 44J), suggesting that PE4 and PE5 can enhance prime editing in MMR-proficient cell types even for classes of edits that evade MMR activity more effectively. Together, this comparison between 70 edits across seven endogenous sites in HEK293T, HeLa, K562, and U2OS cells illustrate that MLH1 dn substantially improves prime editing efficiency, but especially in cell types that are not MMR-deficient.

### Effect of MLH1dn on Prime Editing Outcome Purity

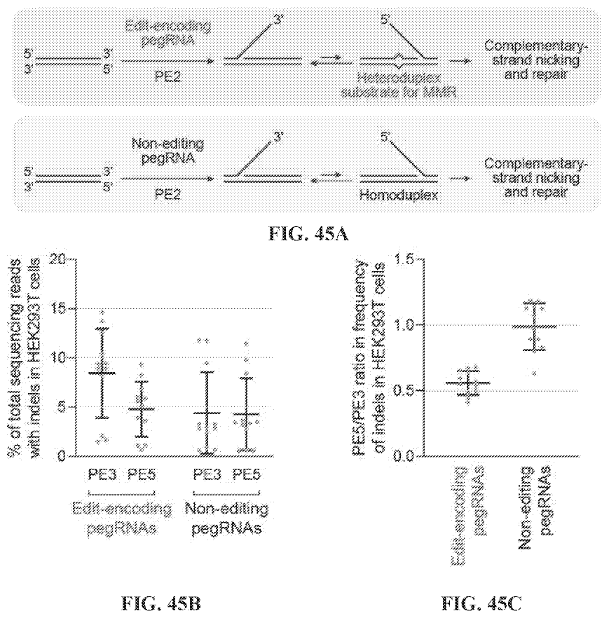

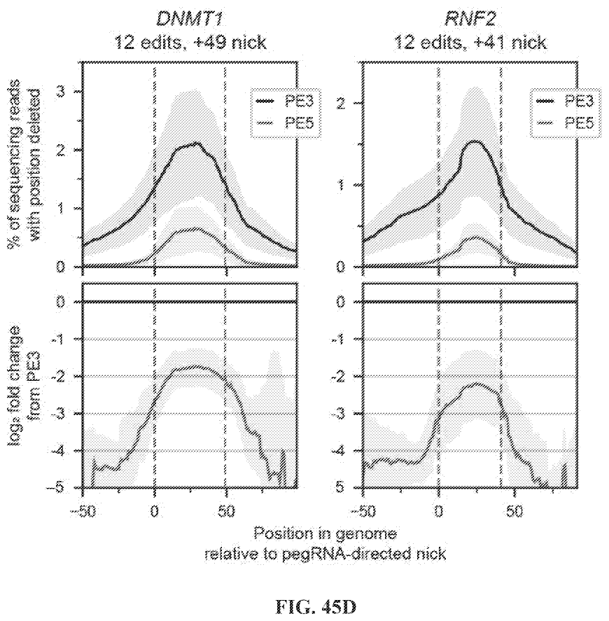

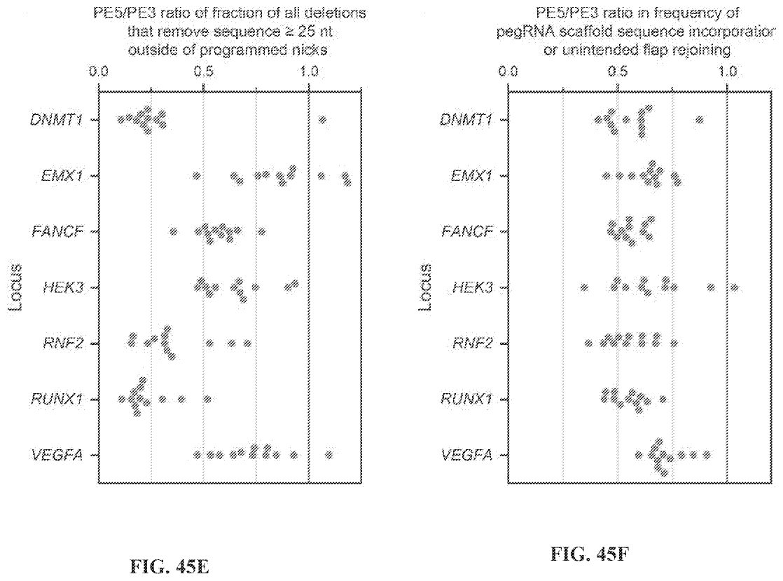

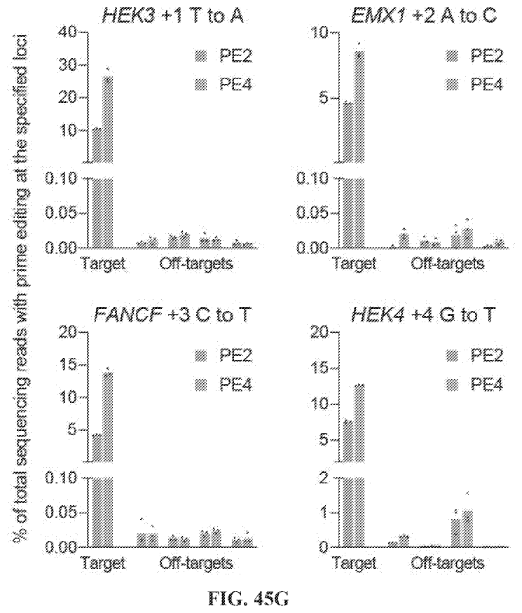

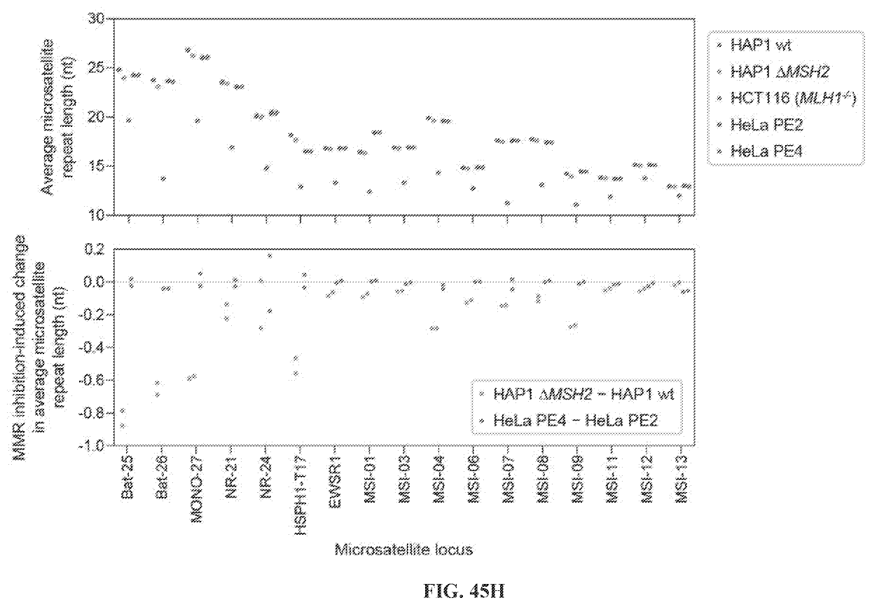

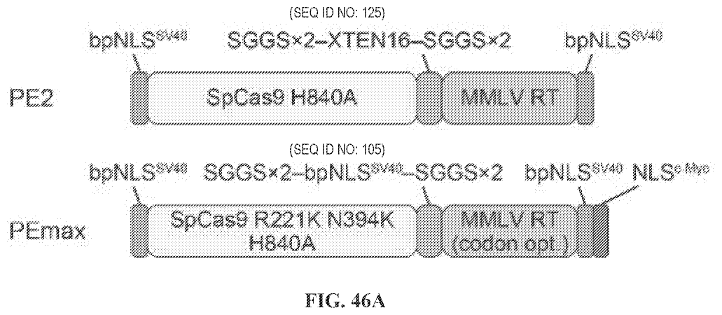

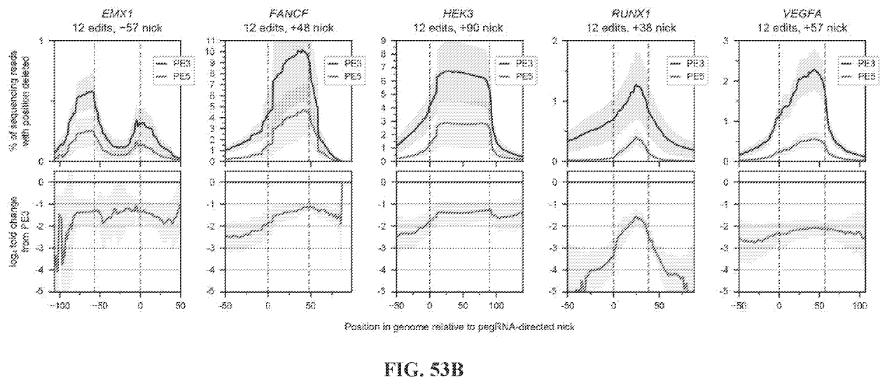

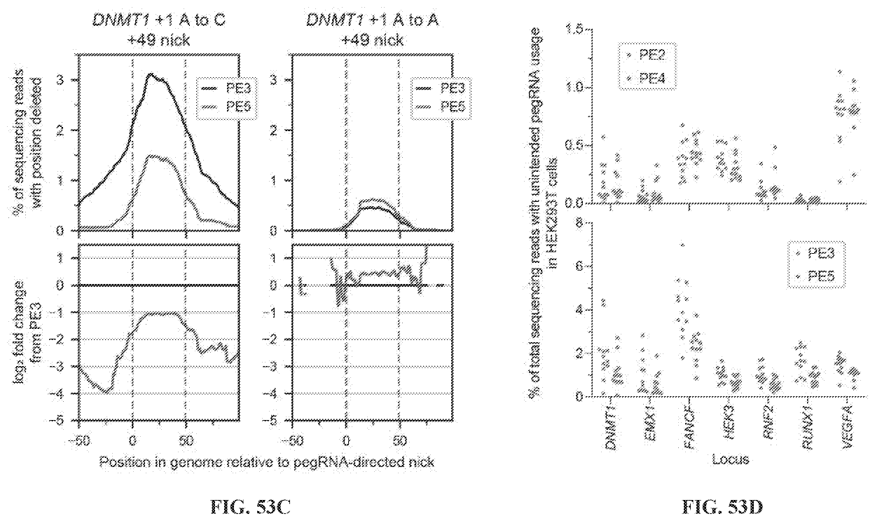

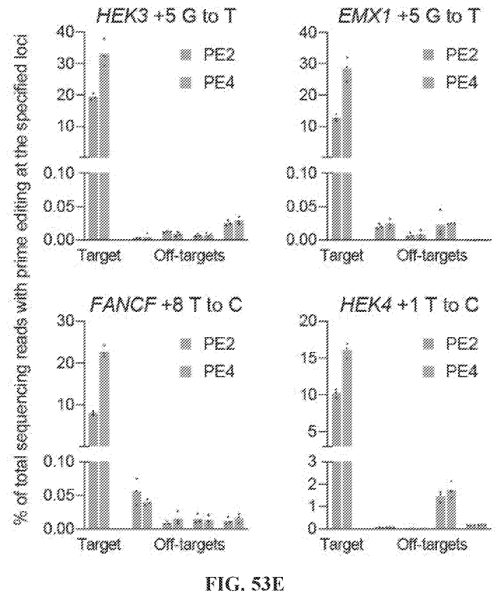

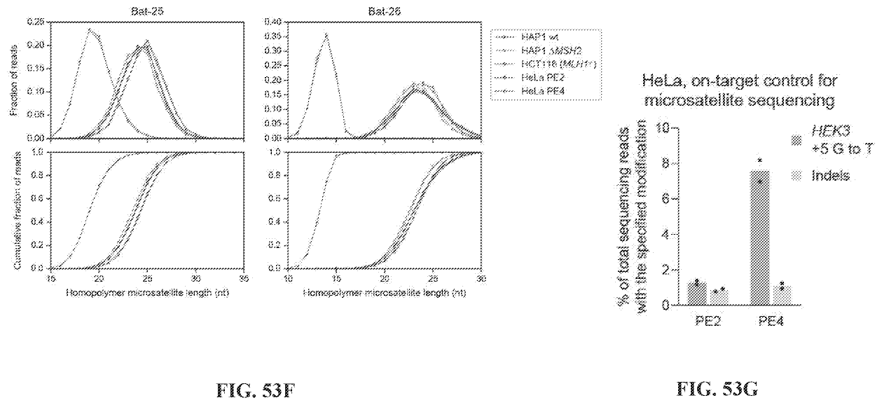

How MLH1dn reduces unintended prime editing outcomes at endogenous genomic targets was characterized next in depth. To decouple steps that lead to prime editing from those that lead to indel byproducts, prime editors were tested with non-editing pegRNAs that template a 3′ DNA flap with perfect complementarity to the target locus and would result in no sequence change at the target locus (FIG. 45A). Across four sites in HEK293T cells, these non-editing pegRNAs yielded an average 4.4% indels with PE3 and 4.3% indels with PE5 (FIGS. 45B-45C, FIG. 53A). In contrast, four pegRNAs that program single-nucleotide mutations at these sites generated on average 8.5% indels with PE3 and 4.8% indels with PE5. These data show that MMR inhibition with MLH1 dn does not alter indel frequencies in the absence of a heteroduplex. It was also observed that MLH1dn did not alter the frequency of indels from pegRNAs and nicking sgRNAs co-transfected with PE2-dRT, a PE2 containing an inactivated reverse transcriptase, or with SpCas9 H840A nickase (nCas9; FIG. 53A), suggesting that MMR does not affect the repair of a doubly nicked intermediate that lacks a reverse-transcribed 3′ flap. These results together strongly suggest that MMR engagement of the prime editing heteroduplex intermediate stimulates indel products that can be mitigated with PE5.

Next, evidence that MMR activity expands the size of prime editing indel byproducts was looked for at seven endogenous loci in HEK293T cells. The distribution of unintended target sequence deletions from PE3 and PE5 was compared with 84 pegRNAs encoding single-base substitutions. Consistent with findings from the Repair-seq screens (FIG. 41I), MLH1dn in the PE5 system reduced deletions outside of the pegRNA- and sgRNA-programmed nicks to a greater extent than deletions between these nicks (FIGS. 45D-45E, 53B). In comparison. MLH1dn did not affect the distribution of prime editing deletion outcomes from a non-editing pegRNA that does not create a mismatch (FIG. 53C). Taken together, this analysis supports a model in which non-programmed incisions by MutLα and subsequent excision at the target locus generates larger indel byproducts during MMR of prime editing intermediates.

Lastly, it was examined how MLH1dn impacts pegRNA scaffold sequence incorporation and unintended flap rejoining, two unintended outcome categories previously identified in the Repair-seq screens (FIGS. 41C-41D). Across the 84 pegRNAs tested in HEK293T cells above, PE5 reduced the average frequency of these combined outcomes by 1.6-fold compared to PE3 (from 1.8% to 1.0% on average, FIG. 45F, FIG. 53D). Consistent with data from PE2 and PE3+50 screens (FIGS. 41E-41F), these outcomes were much rarer in the absence of a complementary-strand nick (0.27% frequency for PE2, 0.28% for PE4; FIG. 53D), suggesting that they typically form through a doubly nicked intermediate. Thus, PE5 broadly narrows the size of unintended deletions and reduces the frequency of pegRNA scaffold sequence incorporation and unintended flap rejoining compared to PE3.

### Effect of MLH1dn on Off-Target Genomic DNA Changes

It was next assessed whether MMR component manipulation could influence off-target editing. PE2 and PE4 were tested in HEK293T cells with eight pegRNAs targeting the HEK3, EMX1, FANCF, and HEK4 loci. The resulting genomic changes were measured at the top four Cas9 off-target sites identified by CIRCLE-seq for each of the targeted loci (Tsai et al., 2017). The average frequency of off-target prime editing remained very low with or without ML1dn (0.094% with PE2, 0.12% with PE4), while the average efficiency of on-target editing increased from 9.7% for PE2 to 20% for PE4; (FIG. 45G, FIG. 53E). These data are consistent with previous reports noting the high DNA specificity of prime editing (Anzalone et al., 2019; Jin et al., 2021; Kim et al., 2020), and suggest that MLH1dn does not substantially increase off-target prime editing.

Next, it was asked whether transient inhibition of MMR with MLH1dn could induce genomic mutations independent of prime editor activity. In humans, MMR deficiency is a risk factor for colorectal cancer and most frequently manifests as mutations altering the length of repetitive microsatellite sequences within the genome (Fishel et al., 1993; Leach et al., 1993; Parsons et al., 1993). Because errors from microsatellite replication are repaired almost exclusively by MMR (Strand et al., 1993; Tran et al., 1997), microsatellite instability is used clinically as a measure of MMR activity (Bacher et al., 2004; Umar et al., 2004).

Microsatellite instability was evaluated by high-throughput sequencing of 17 mononucleotide tracts previously shown to be responsive to MMR inhibition and used as clinical biomarkers for MMR deficiency (Hempelmann et al., 2015). These microsatellite loci were analyzed in HAP1 cells, HeLa cells and in MMR-deficient HCT1 16 cells. HCT116 cells exhibited substantially shorter microsatellite lengths on average (13.9-nt) than HAP1 or HeLa cells (18.4-nt; FIG. 45H, FIG. 53F). To gauge whether this assay can also detect a shorter duration of MMR deficiency in the same cell type, microsatellite instability was compared in wild-type HAP1 cells and monoclonal HAP1 cells grown for 2 months (˜60 cell divisions) following knockout of MSH2. These MMR knockout cells exhibited a 0.24-nt average decrease in microsatellite length (FIG. 45H, FIG. 53F), demonstrating that even recent MMR impairment can be detected through the accumulation of microsatellite length erosion.

To assess the effect of transient MLH1dn expression as used in PE4 and PE5 systems, microsatellite instability was next measured in MMR-proficient HeLa cells 3 days after transfection with plasmids encoding PE2 or PE4. Although MLH1dn improved prime editing efficiency from 1.3% (PE2) to 7.6% (PE4) at the on-target locus (FIG. 51G), average microsatellite lengths were indistinguishable in PE2-treated cells compared to in PE4-treated cells (<0.01 nt of difference; FIG. 45H, FIG. 53F). These data indicate that transient MLH1dn expression can enhance prime editing without stimulating detectable instability at 17 microsatellites used for clinical diagnosis of MMR deficiency. As microsatellite repeats are up to 10⁵-fold more susceptible to MMR inhibition than the rest of the genome on average (Strand et al., 1993; Tran et al., 1997), these findings suggest that the PE4 and PE5 editing systems that use transient MLH1dn expression, including MLH1dn mRNA co-delivery described below, can enhance on-target editing without substantial off-target mutational burden.

PEmax Systems with Optimized Architectures and Synergy with Engineered pegRNAs

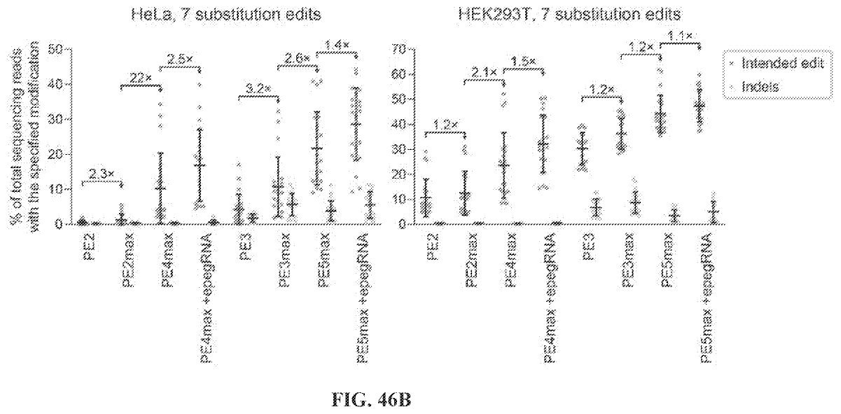

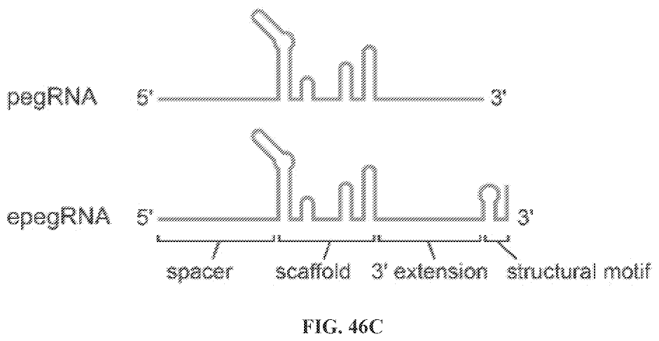

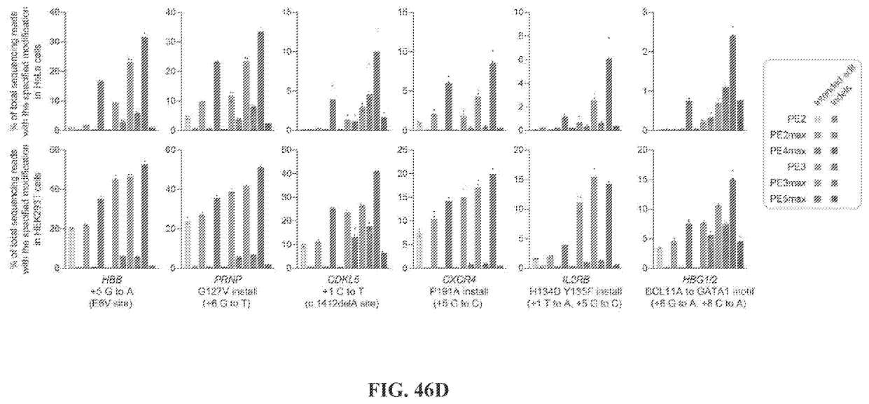

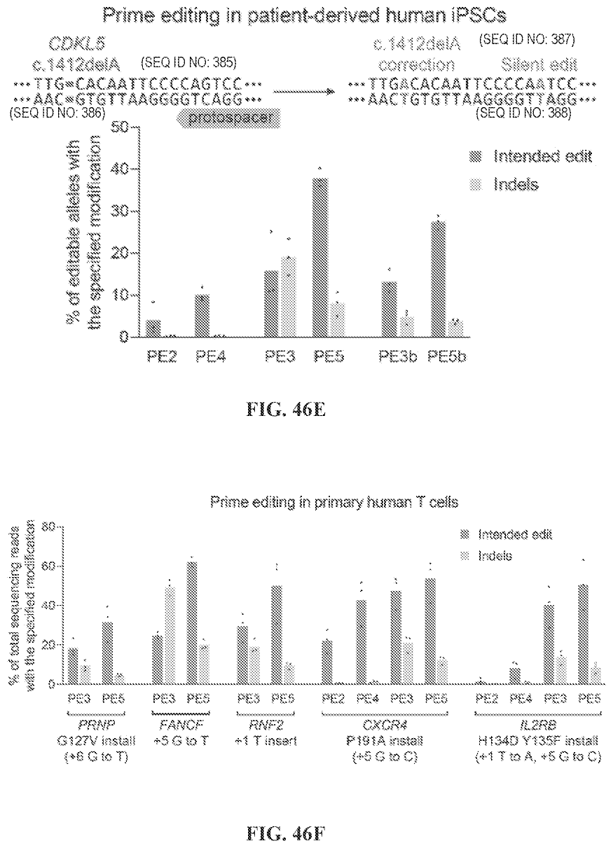

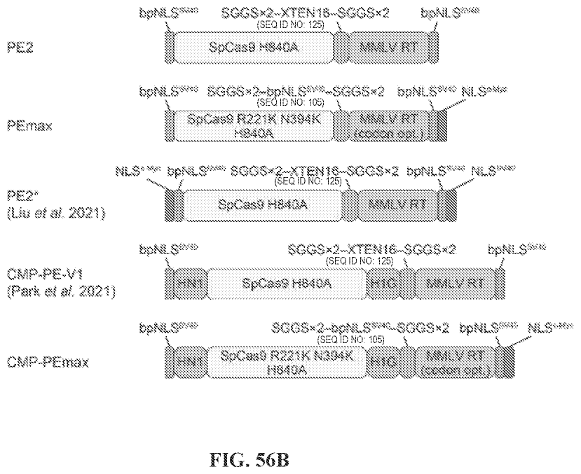

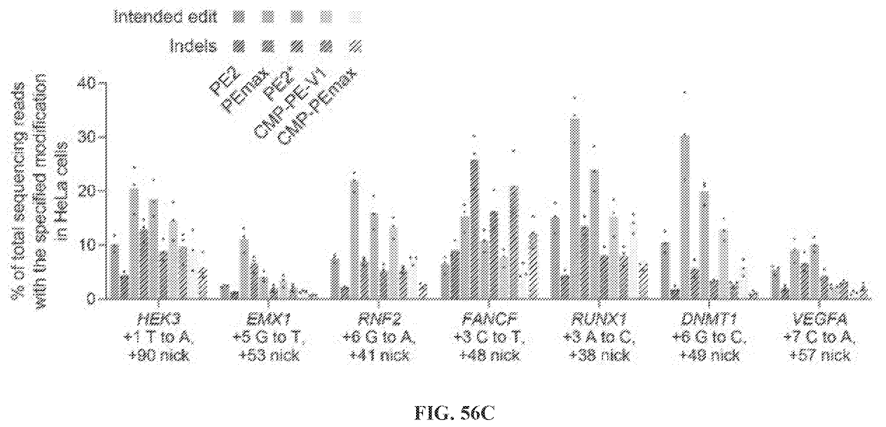

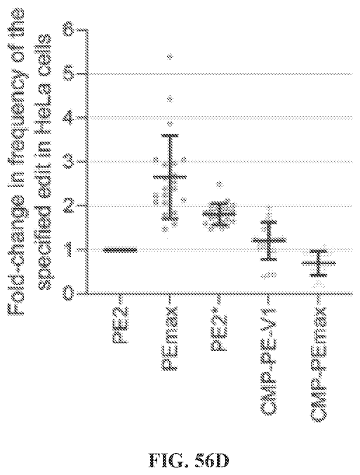

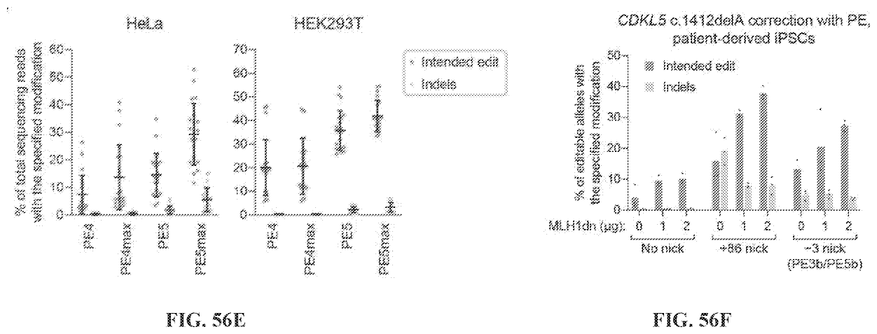

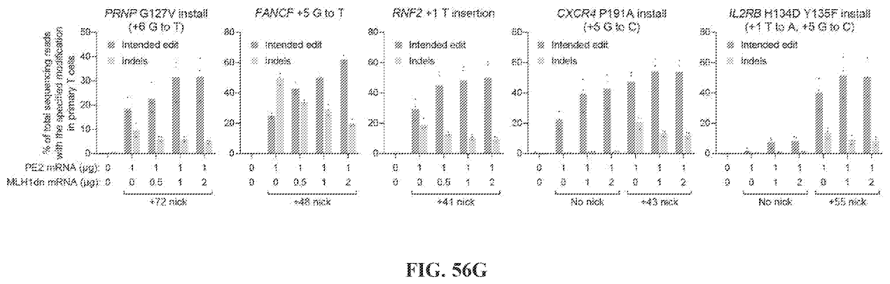

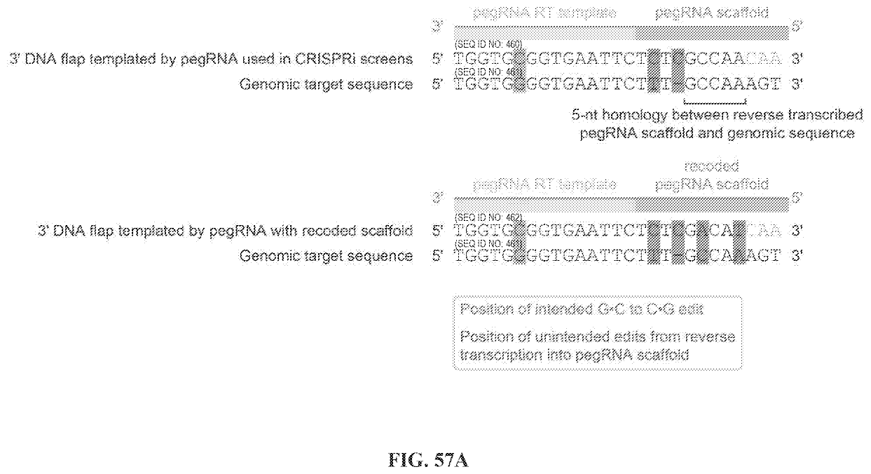

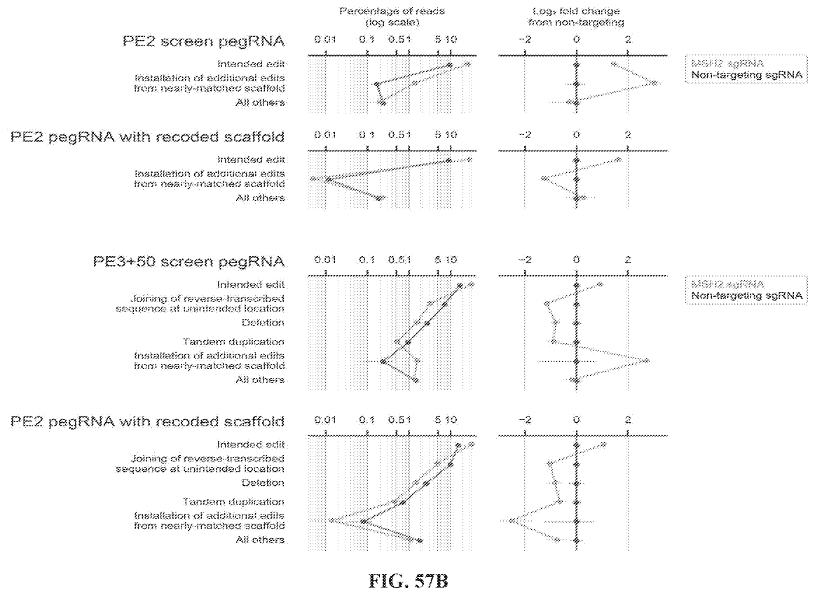

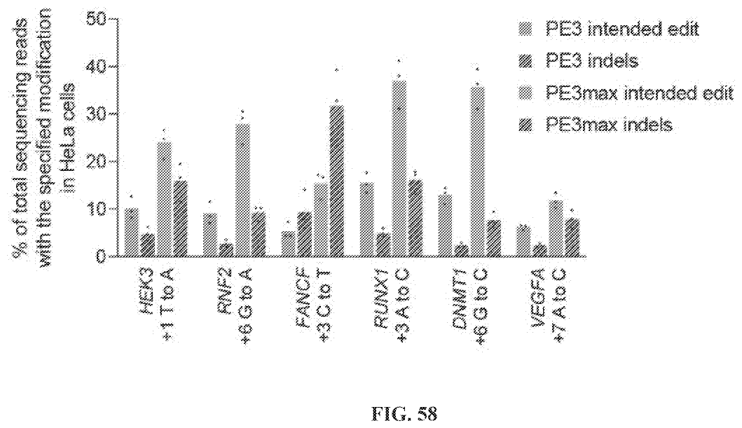

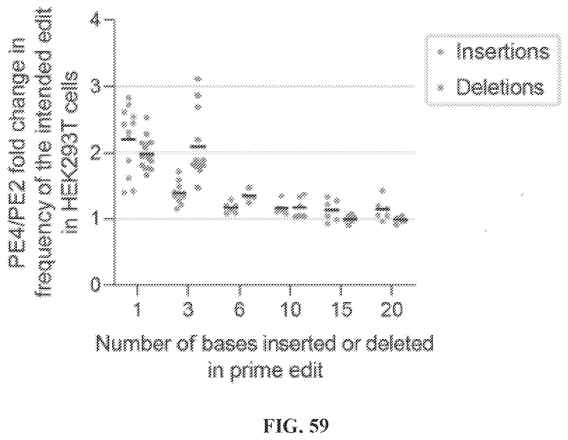

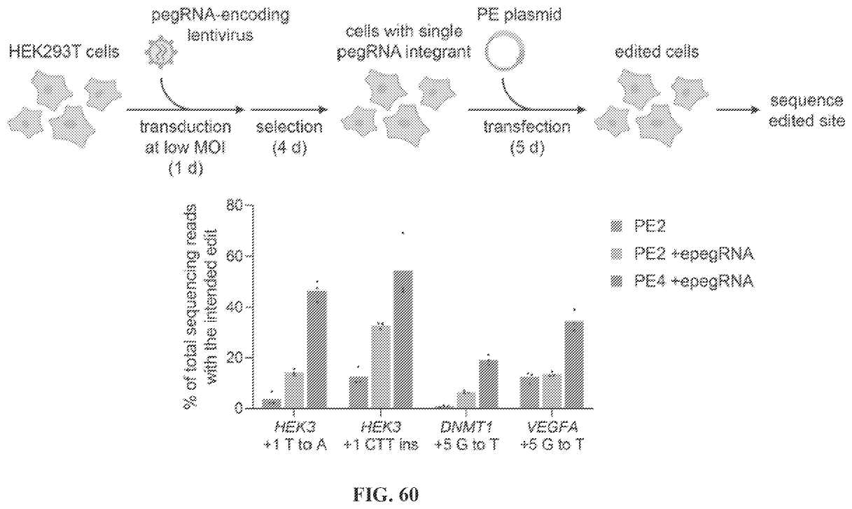

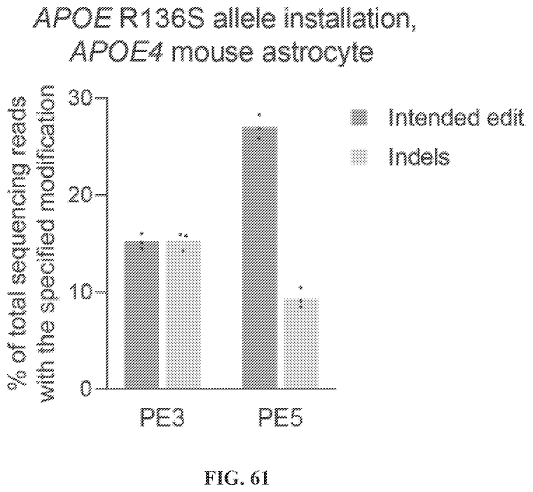

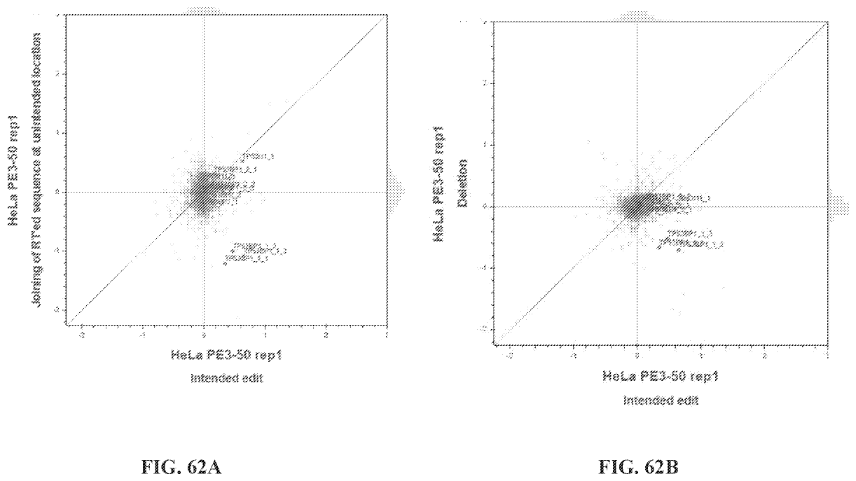

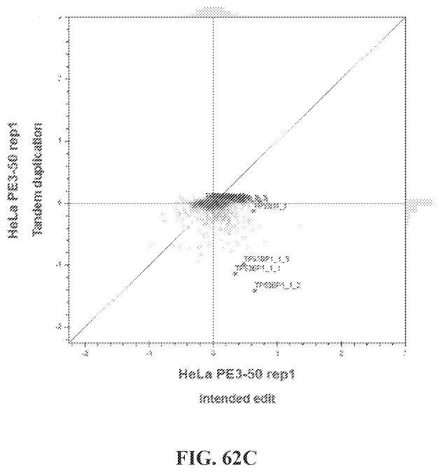

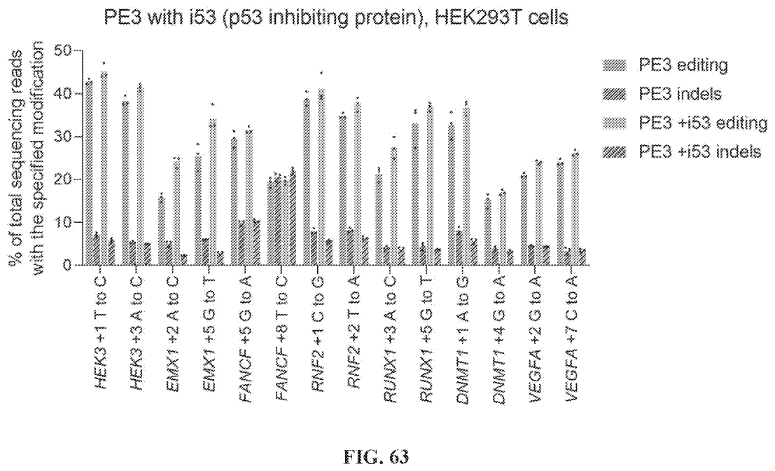

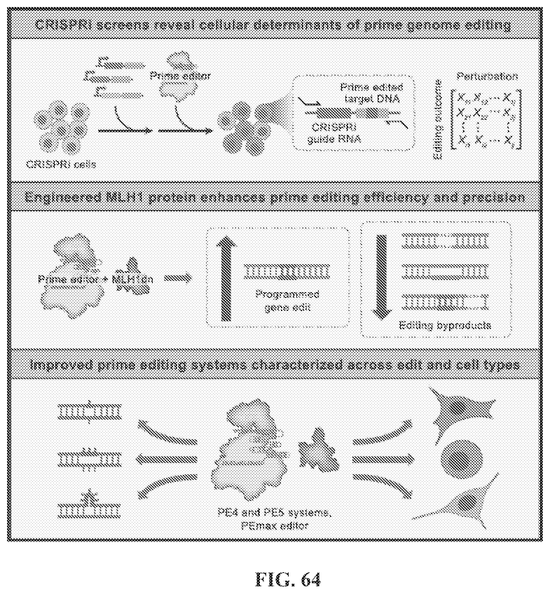

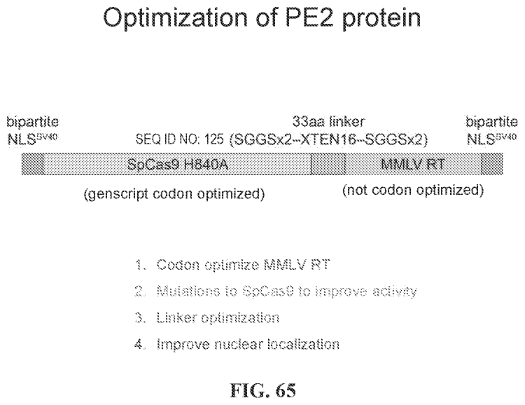

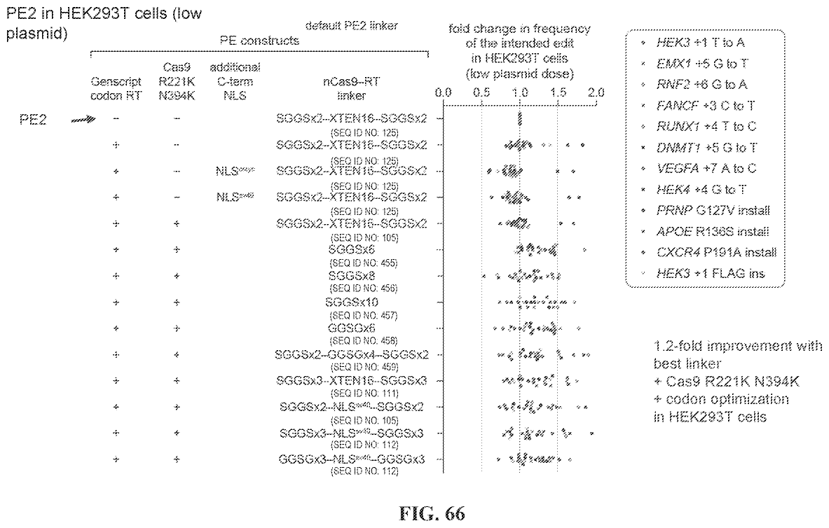

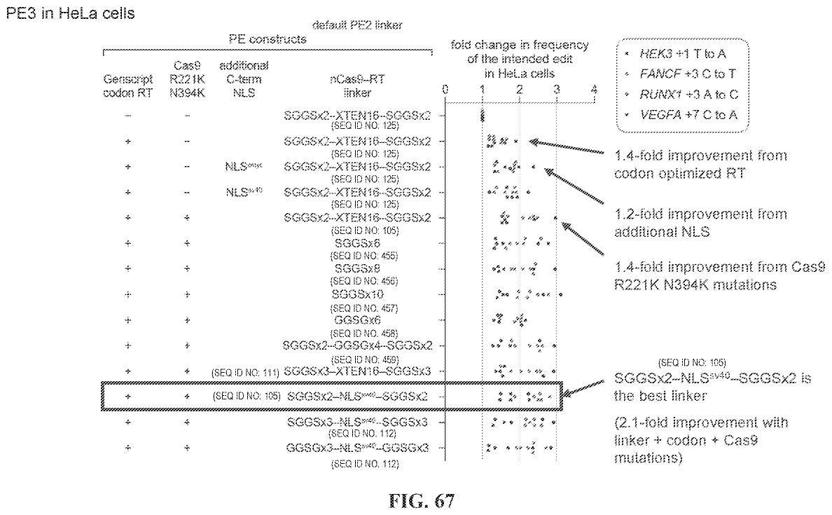

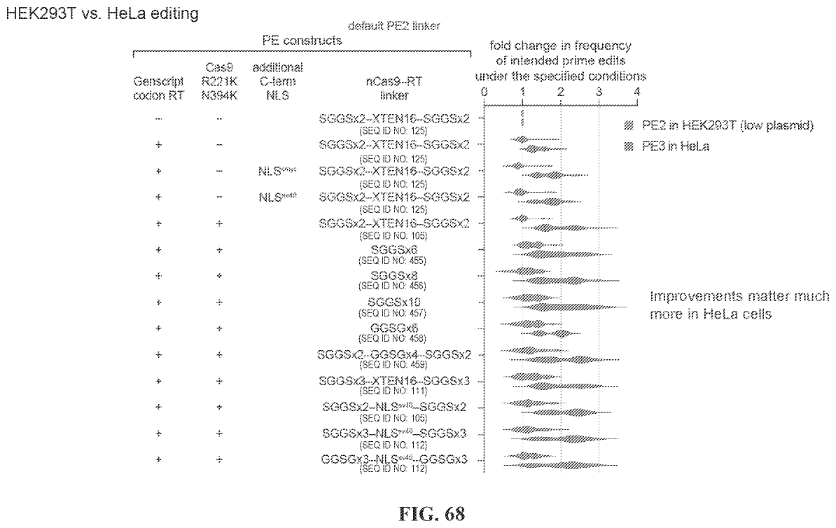

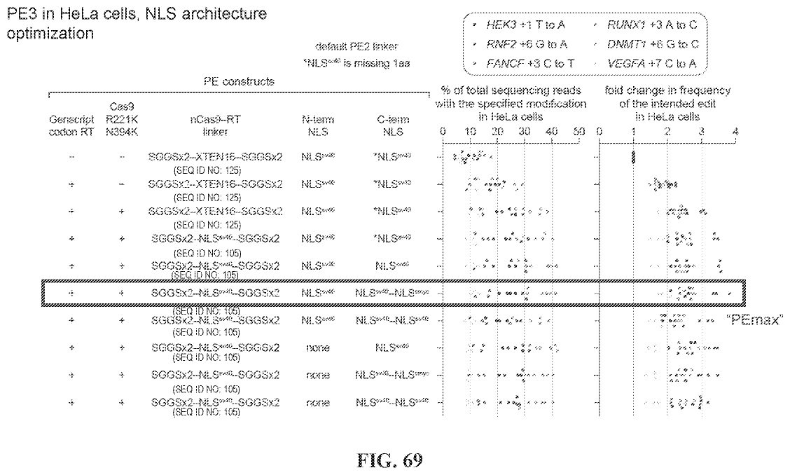

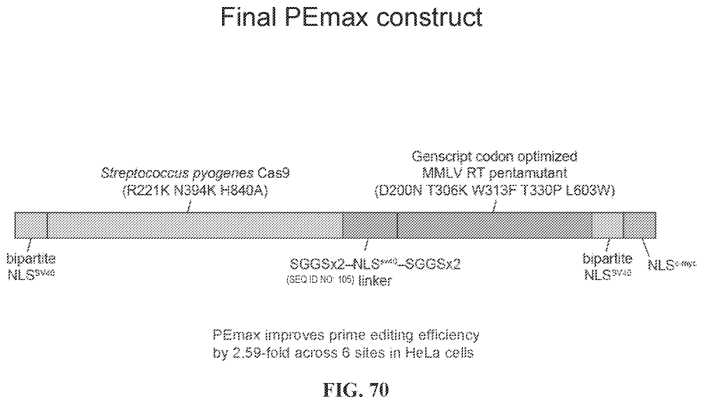

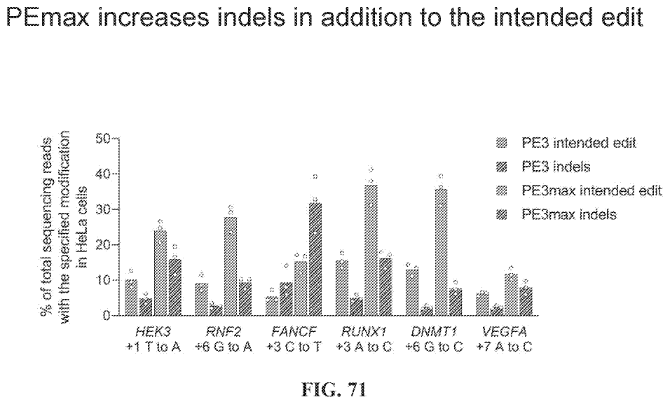

To further improve prime editing, the PE2 protein was optimized by varying RT codon usage, mutations within the SpCas9 domain, the length and composition of peptide linkers between nCas9 and RT, and the location, composition, and number of NLS sequences (FIGS. 56A-56B). Among 21 such variants tested, the greatest enhancement in editing efficiency was observed from a prime editor architecture that uses a human codon-optimized RT, a 34-aa linker containing a bipartite SV40 NLS (Wu et al., 2009), an additional C-terminal c-Myc NLS (Dang and Lee, 1988), and R221K N394K mutations in SpCas9 previously shown to improve Cas9 nuclease activity (Spencer and Zhang, 2017) (FIG. 46A, FIG. 56A). This optimized prime editor architecture was designated as PEmax (SEQ ID NO: 99 disclosed herein; also see schematic of FIG. 54A). At seven target sites tested in HeLa cells, this optimized prime editor architecture (hereafter referred to as PEmax) outperforms other improved prime editor variants, including PE2*, which includes additional NLS sequences (Liu et al., 2021), and CMP-PE-V1, which contains high-mobility peptides (Park et al., 2021) (FIGS. 56B-56D). Inserting high-mobility peptides into PEmax (CMP-PEmax) did not further improve prime editing (FIGS. 56C-56D).

Across seven substitution edits targeting different loci, using the PEmax architecture with the PE2 system (PE2max) increased the average frequency of intended editing by 2.3-fold in HeLa cells and 1.2-fold in HEK293T cells over the original PE2 architecture (FIG. 52B). Similarly, PE3 using the PEmax architecture (PE3max) increased average editing efficiency over PE3 by 2.8-fold in HeLa cells and 1.2-fold in HEK293T cells (FIGS. 53B and 56C). PE3max also slightly reduced average edit:indel purity by 1.2-fold in both cell types, which may reflect enhanced nickase activity from the Cas9 R221K and N394K mutations within the PEmax architecture (Spencer and Zhang, 2017).

It was also observed that PE4max (PE4 using the PEmax architecture) enhanced average editing frequencies over PE4 by 1.9-fold in HeLa cells and by 1.1-fold in HEK293T cells (FIG. 56E). Finally, PE5max (PE5 using the PEmax architecture) similarly increased average editing efficiency over PE5 by 2.2-fold in HeLa cells and 1.2-fold in HEK293T cells.

In a separate improvement to prime editing, engineered pegRNAs (epegRNAs) were recently developed, which contain an additional 3′ RNA structural motif that increases prime editing efficacy (Nelson et al., 2021) (FIG. 46C). To assess whether optimized PE4max and PE5max systems can synergize with epegRNAs. PE4max and PE5max were tested in combination with epegRNAs in HeLa and HEK293T cells. Across seven substitution edits targeting different loci, epegRNAs improved PE4max editing efficiency over normal pegRNAs by an average of 2.5-fold in HeLa cells and 1.5-fold in HEK293T cells (FIG. 46B). Similarly, epegRNAs enhanced PE5max editing over normal pegRNAs by 1.4-fold in HeLa cells and 1.1-fold in HEK293T cells, without affecting edit:indel purity.

Combining all enhancements to prime editing systems described above—MLH1dn, PEmax, and epegRNAs—dramatically improved prime editing performance. PE4max with epegRNAs enhanced editing efficiency by an average of 72-fold in MMR-proficient HeLa cells and 3.5-fold in MMR-deficient HEK293T cells relative to PE2 with normal pegRNAs (FIG. 46B). PE5max with epegRNAs also improved editing efficiency over PE3 with pegRNAs on average by 12-fold in HeLa cells and 1.6-fold in HEK293T cells, with outcome purity increasing on average by 4.6-fold in HeLa cells and 3.3-fold in HEK293T cells. Collectively, these results demonstrate that combining PE4/PE5. PEmax, and epegRNA approaches can greatly enhance prime editing efficiency and outcome purity.

Prime Editing of Disease-Relevant Loci and Cell Types with PE4 and PE5

To establish the applicability of these improved editing systems, PE4max and PE5max were applied to edit therapeutically relevant loci in wild-type HeLa and HEK293T cells. First, a silent G⋅C-to-A⋅T transversion was made at the 6^{th} codon of HBB, which is mutated in sickle cell disease patients (Ingram, 1956). Second, the G127V allele (a G⋅C-to-T⋅A transversion) was installed in PRNP that confers resistance to prion disease (Asante et al., 2015; Mead et al., 2009). Third, a silent C⋅G-to-T⋅A mutation was introduced at a CDKL5 site known to contain a causative mutation for CDKL5 deficiency disorder, a severe neurodevelopmental condition (Olson et al., 2019). Fourth, the CXCR4 P191A allele (a G⋅C-to-C⋅G edit) that inhibits HIV infection in human cells was installed (Liu et al., 2018). Fifth, the IL2RB H134D Y135F (non-adjacent T⋅A-to-A⋅T and G⋅C-to-C⋅G edits) variant that enables orthogonal IL-2 receptor responsiveness for adoptive T cell transfer therapy was generated (Sockolosky et al., 2018). Lastly, the BCL 11 A repressor binding site within the HBG1 and HBG2 fetal hemoglobin gene promoters was recoded to a GATA1 transcriptional activator motif (non-adjacent G⋅C-to-A⋅T and C⋅G-to-A⋅T edits), which in principle could induce fetal hemoglobin expression for treatment of hemoglobinopathies (Amato et al., 2014). Sequences are shown in Table 4, below.

TABLE 4
Sequences Used for Prime Editing of Disease-Relevant Loci and Cell Types

| | | SEQ |
|---|---|---|
| Oligonucleotides | Sequence | ID NO: |
| CDKL5 | mG*mA*mG*rGrGrArCrUrCrCrUrArGrArGrGrArCrUrGrGrUrUrUrUrArGrArGrCrUr | 237 |
| c.1412delA | ArGrArArArUrArGrCrArArGrUrUrArArArArUrArArGrGrCrUrArGrUrCrCrGrUrUrAr | |
| correction with | UrCrArArCrUrUrGrArArArArArGrUrGrGrCrArCrCrGrArGrUrCrGrGrUrGrCrArUrAr | |
| silent edit | UrUrGrArCrArCrArArUrUrCrCrCrCrArArUrCrCrUrCrUrArGrGrArGrUrCrArArArAr | |
| epegRNA | ArCrArCrGrUrCrArGrGrGrUrCrArGrGrArGrCrCrCrCrCrCrCrCrCrUrGrCrArCrCrCr | |
| | ArGrGrArArArArCrCrCrUrCrArArArGrUrCrGrGrGrGrGrGrCrArA*mC*mC*mC | |
| CDKL5 +86 nick | mG*mC*mA*rGrArArCrCrGrCrCrArCrUrCrArUrUrCrArGrUrUrUrUrArGrArGrCrUr | 238 |
| sgRNA | ArGrArArArUrArGrC:ArArGrUrUrArArArArUrArArGrGrCrUrArGrUrCrCrGrUrUrAr | |
| | UrCrArArCrUrUrGrArArArArArGrUrGrGrCrArCrCrGrArGrUrCrGrGrUrGrCrUmU*m | |
| | U*mU | |
| CDKL5 −3 nick | mA*mC*mA*rCrArArUrUrCrCrCrCrArArUrCrCrUrCrUrGrUrUrUrUrArGrArGrCrUr | 239 |
| sgRNA (for | ArGrArArArUrArGrCrArArGrUrUrArArArArUrArArGrGrCrUrArGrUrCrCrGrUrUrAr | |
| PE3b/PE5b) | UrCrArArCrUrUrGrArArArArArGrUrGrGrCrArCrCrGrArGrUrCrGrGrUrGrCrUmU*m | |
| | U*mU | |
| PRNP G127V | mG*mC*mA*rGrUrGrGrUrGrGrGrGrGrGrCrCrUrUrGrGrGrUrUrUrUrArGrArGrCrUr | 240 |
| install pegRNA | ArGrArArArUrArGrCrArArGrUrUrArArArArUrArArGrGrCUrArGrUrCrCrGrUrUrAr | |
| | UrCrArArCrUrUrGrArArArArArGrUrGrGrCrArCrCrGrArGrUrCrGrGrUrGrCrArUrGr | |
| | UrArGrArCrGrCrCrArArGrGrCrCrCrCrCrC*mA*mC*mC | |
| PRNP +72 nick | mG*mC*mA*rUrGrUrUrUrUrCrArCrGrArUrArGrUrArArGrUrUrUrUrArGrArGrCrUr | 241 |
| sgRNA | ArGrArArArUrArGrC:ArArGrUrUrArArArArUrArArGrGrCrUrArGrUrCrCrGrUrUrAr | |
| | UrCrArArCrUrUrGrArArArArArGrUrGrGrCrArCrCrGrArGrUrCrGrGmUrGrCrUmU* | |
| | mU*mU | |
| FANCF +5 G to | mG*mG*mA*rArUrCrCrCrUrUrCrUrGrCrArGrCrArCrCrGrUrUrUrUrArGrArGrCrUr | 242 |
| T pegRNA | ArGrArArArUrArGrCrArArGrUrUrArArArArUrArArGrGrCrUrArGrUrCrCrGrUrUrAr | |
| | UrCrArArCrUrUrGrArArArArArGrUrGrGrCrArCrCrGrArGrUrCrGrGrUrGrCrGrGrAr | |
| | ArArArGrCrGrArUrCrArArGrGrUrGrCrUrGrCrArGrArArG*mG*mG*mA | |
| FANCF +48 nick | mG*mG*mG*rGrUrCrCrCrArGrGrUrGrCrUrGrArCrGrUrGrUrUrUrUrArGrArGrCrUr | 243 |
| sgRNA | ArGrArArArUrArGrCrArArGrUrUrArArArArUrArArGrGrCrUrArGrUrCrCrGrUrUrAr | |
| | UrCrArArCrUrUrGrArArArArArGrUrGrGrCrArCrCrGrArGrUrCrGrGrUrGrCrUmU*m | |
| | U*mU | |
| RNF2 +1 T | mG*mU*mC*rArUrCrUrUrArGrUrCrArUrUrArCrCrUrGrGrUrUrUrUrArGrArGrCrUr | 244 |
| insertion | ArGrArArArUrArGrCrArArGrUrUrArArArArUrArArGrGrCrUrArGrUrCrCrGrUrUrAr | |
| pegRNA | UrCrArArCrUrUrGrArArArArArGrUrGrGrCrArCrCrGrArGrUrCrGrGrUrGrCrArArCr | |
| | GrArArCrArCrCrUrCrArGrArGrUrArArUrGrArCrUrArArG*mA*mU*mG | |
| RNF2 +41 nick | mU*mC*mA*rArCrCrArUrUrArArGrCrArArArArCrArUrGrUrUrUrUrArGrArGrCrUr | 245 |
| sgRNA | ArGrArArArUrArGrCrArArGrUrUrArArArArUrArArGrGrCrUrArGrUrCrCrGrUrUrAr | |
| | UrCrArArCrUrUrGrArArArArArGrUrGrGrCrArCrCrGrArGrUrCrGrGrUrGrCrUmU*m | |
| | U*mU | |
| CXCR4 P191A | mC*mA*mA*rCrCrArCrCrCrArCrArArGrUrCrArUrUrGrGrUrUrUrUrArGrArGrCrUr | 246 |
| install pegRNA | ArGrArArArUrArGrCrArArGrUrUrArArArArUrArArGrGrCrUrArGrUrCrCrGrUrUrAr | |
| | UrCrArArCrUrUrGrArArArArArGrUrGrGrCrArCrCrGrArGrUrCrGrGrUrGrCrUrGrAr | |
| | CrCrGrCrUrUrCrUrArCrGrCrCrArArUrGrArCrUrUrGrUrGrGrGrU*mG*mG*mU | |
| CXCR4 +43 nick | mC*mA*mU*rCrUrUrUrGrCrCrArArCrGrUrCrArGrUrGrGrUrUrUrUrArGrArGrCrUr | 247 |
| sgRNA | ArGrArArArUrArGrCrArArGrUrUrArArArArUrArArGrGrCrUrArGrUrCrCrGrUrUrAr | |
| | UrCrArArCrUrUrGrArArArArArGrUrGrGrCrArCrCrGrArGrUrCrGrGrUrGrCrUmU*m | |
| | U*mU | |
| IL2RB H134D | mC*mC*mA*rGrGrUrGrUrCrUrUrUrCrArArArGrUrArGrGrUrUrUrUrArGrArGrCrUr | 248 |
| Y135F install | ArGrArArArUrArGrCrArArGrUrUrArArArArUrArArGrGrCrUrArGrUrCrCrGrUrUrAr | |
| pegRNA | UrCrArArCrUrUrGrArArArArArGrUrGrGrCrArCrCrGrArGrUrCrGrGrUrGrCrUrCrCr | |
| | CrArArGrCrCrUrCrCrGrArCrUrUrCrUrUrUrGrArArArGrA*mC*mA*mC | |
| IL2RB +55 nick | mC*mU*mC*rCrCrUrCrCrArArGrUrUrGrUrCrCrArCrGrGrUrUrUrUrArGrArGrCrUr | 249 |
| sgRNA | ArGrArArArUrArGrCrArArGrUrUrArArArArUrArArGrGrCrUrArGrUrCrCrGrUrUrAr | |
| | UrCrArArCrUrUrGrArArArArArGrUrGrGrCrArCrCrGrArGrUrCrGrGrUrGrCrUmU*m | |
| | U*mU | |

Across these six disease-relevant edits, it was observed that PE4max increased average prime editing efficiency over PE2 by 29-fold in HeLa cells and 2.1-fold in HEK293T cells (FIG. 46D). Notably, PE4max editing efficiencies (8.6% editing with 0.19% indels in HeLa cells, and 20% editing with 0.26% indels in HEK293T cells) were similar to or exceeded those of PE3 (4.5% editing with 1.5% indels in HeLa cells, and 24% editing with 5.4% indels in HEK293T cells), but with far fewer indels. In addition. PE5max improved disease-relevant allele conversion over PE3 by an average of 6.1-fold in HeLa cells and 1.5-fold in HEK293T cells, and enhanced edit:indel purity by 6.4-fold in HeLa cells and by 3.5-fold in HEK293T cells (FIG. 46D). Taken together, these results demonstrate that PE4max and PE5max support substantially higher prime editing performance compared to PE2 and PE3 at gene targets relevant to human disease in commonly used cell lines.

Next, PE4 and PE5 editing systems were evaluated in a cell model of genetic disease and in primary human cells. First, the pathogenic CDKL5 c.1412delA mutation in human induced pluripotent stem cells (iPSCs) derived from a patient heterozygous for this allele was corrected (Chen et al., 2021). Electroporation of these iPSCs with PE3 components (in vitro-transcribed PE2 mRNA and synthetic pegRNAs and nicking sgRNAs) yielded 17% correction of editable pathogenic alleles and 20% total indel products (FIG. 46E, FIG. 56F). Co-electroporation of these components with MLH1dn mRNA for PE5 editing elevated correction efficiency to 34% and lowered the frequency of indels to 6.1%. To further minimize indels, PE4 and PE5b strategies were also used. Without complementary-strand nicking, it was observed that MLH1dn improved allele correction from 4.0% (PE2) to 10% (PE4) with few indels (<0.34%) (FIG. 46E, FIG. 56F). Similarly, PE3b resulted in 13% editing of the mutant allele with 4.8% indels, while PE5b elevated editing to 27% with 3.8% indels. Across the PE4, PE5, and PE5b systems tested, MLH1dn enhances correction of the pathogenic CDKL5 c.1412delA mutation by 2.2-fold in efficiency and 3.6-fold in outcome purity in patient-derived iPSCs.

Second, primary T cells were electroporated from healthy human donors with PE2 mRNA, MLH1dn mRNA, and synthetic pegRNAs and nicking sgRNAs to introduce the protective PRNP G127V mutation, a G⋅C-to-T⋅A transversion at FANCF, or a 1-bp insertion of a T at RNF2. At these three sites, it was found that PE5 achieved an average of 41% editing efficiency with 12% indels, a substantial improvement from 22% editing efficiency and 26% indels with PE3 (FIG. 46F, FIG. 56G). PE4 and PE5 were also applied to install the protective CXCR4 P191A allele that prevents HIV infection (Liu et al., 2018), and the IL2RB H134D Y135F variant that enables orthogonal IL-2 T cell stimulation (Sockolosky el al., 2018). For these edits, PE4 increased the frequency of allele conversion over PE2 by an average of 3.6-fold with few indel byproducts (FIG. 46F. FIG. 56G). In addition, PE5 resulted in an average of 52% editing with 11% indels, compared to 44% editing with 17% indels with PE3, a 2.0-fold improvement in edit:indel ratio. Taken together, these results across six sites in human iPSCs and primary T cells establish PE4 and PE5 as enhanced prime editing systems that enable substantially greater editing efficiency and outcome purity in cell types relevant to the study and potential treatment of genetic disease.

### Discussion

Using pooled CRISPRi screens, it was discovered that MMR activity strongly suppresses the efficiency and outcome purity of substitution prime edits. Based on the results and insights from the CRISPRi screens and the role of MMR in suppressing editing with nucleotide substitution, the PE4 and PE5 systems were developed. Particularly, the PE4 and PE5 systems co-express MLH1dn to transiently inhibit MMR, enhance prime editing efficacy, and reduce indels without inducing substantial off-target genomic changes. Optimization of the prime editor protein resulted in a PEmax architecture that can synergize with PE4 and PE5 systems, as well as with epegRNAs (Nelson et al., 2021—incorporated herein by reference), to further enhance prime editing performance. Together, the model for DNA repair of prime editing supported by these findings, the PE4 and PE5 strategies developed to circumvent a prime editing bottleneck, and the improved PEmax prime editor architecture described here substantially advance the utility of prime editing for precision manipulation of the genome.

This work revealed that prime editing can install certain types of edits, including G⋅C-to-C⋅G transversions and substitutions of three or more contiguous bases, with higher efficiency due to the ability of the corresponding prime editing intermediates to evade MMR. In addition to edit type, other properties could also affect the sensitivity of prime editing to MMR, such as the sequence context of the target site. The state of the edited locus may also be important, as MMR more efficiently repairs early replicating euchromatin (Supek and Lehner, 2015) as well as lagging strand DNA during replication (Lujan et al., 2014).

While it is demonstrated that co-delivery of MLH1dn with prime editors enhances editing efficiency and precision, the studies presented herein on the role of MMR in determining prime editing outcomes coupled with insights into the types of prime editing intermediates that are repaired by MMR allows researchers to design prime editing experiments to evade MMR, even without expression of MLH1dn. For example, the data presented herein show that strategically installing additional nearby silent mutations can enhance prime editing outcomes by avoiding MMR reversal of prime editing intermediates, even with PE2 or PE3 systems. Other modalities for MMR inhibition may also prove beneficial for prime editing. Although no small molecules that selectively target MMR have been reported, chemical inhibitors would be useful in applications limited by MLH1dn delivery and could allow temporal control of MMR inhibition. For uses such as viral delivery that may maintain prime editor expression over a long duration, RNA interference offers another strategy for transient MMR knockdown.

PE4 and PE5 systems powerfully enhance prime editing in seven mammalian cell types tested, including patient-derived iPSCs and primary T cells. PE4 increases editing efficiency by an average of 7.7-fold over PE2 in MMR-proficient cell types, uniquely enabling appreciable levels of gene editing without generating DSBs from complementary-strand nicking. This improvement in combination with the PEmax architecture and epegRNAs can surpass editing frequencies of PE3 while maintaining few indel byproducts. PE4max with epegRNAs thus will be particularly useful for gene editing applications that cannot tolerate indel formation or are limited by delivery of nicking sgRNAs. In comparison, PE5 elevates editing efficiency over PE3 by an average 2.0-fold and editing outcome purity by approximately 3-fold in cells with active MMR, which includes most cell targets.

### Experimental Model and Subject Details

Culture conditions for immortalized cell lines

HEK293T, HeLa dCas9-BFP-KRAB, HeLa, HCT116, and N2A cells were cultured in Dulbecco's Modified Eagle Medium (DMEM) plus GlutaMAX (Thermo Fisher Scientific) supplemented with 10% fetal bovine serum (FBS) (Thermo Fisher Scientific). HeLa dCas9-BFP-KRAB cells were cultured in DMEM plus GlutaMAX supplemented with 10% FBS. 100 U mL-1 penicillin, and 100 μg mL-1 streptomycin (Thermo Fisher Scientific). K562 dCas9-BFP-KRAB and K562 cells were cultured in Roswell Park Memorial Institute (RPMI) 1640 medium (Thermo Fisher Scientific) supplemented with 10% FBS and 1% Penicillin-Streptomycin-Glutamine 100× (Thermo Fisher Scientific). All HAP1 cell types were cultured in Iscove's Modified Dulbecco's Medium (IMDM) plus GlutaMAX (Thermo Fisher Scientific) supplemented with 10% FBS. U2OS cells were cultured in McCoy's 5A medium (Gibco) supplemented with 10% FBS, 100 U mL⁻¹ penicillin, and 100 μg mL⁻¹ streptomycin (Thermo Fisher Scientific). HeLa dCas9-BFP-KRAB and K562 dCas9-BFP-KRAB cell lines were verified by short tandem repeat marker testing. All cell types were passaged every 2-3 days, maintained below 80% confluency, cultured at 37° C. with 5% CO₂, and tested negative for mycoplasma.

### Isolation of Primary Human T Cells

Peripheral blood mononuclear cells (PBMCs) were isolated from buffy coat of healthy donors (Memorial Blood Centers in St. Paul, Minnesota) by density centrifugation using Lymphoprep density gradient medium (STEMCELL Technologies) and SepMate tubes (STEMCELL Technologies). T cells were isolated from PBMCs using the EasySep Human T Cell Isolation Kit (STEMCELL Technologies).

### Culture Conditions for Human Patient-Derived Induced Pluripotent Stem Cells

All iPSC culturing work was performed by staff at the Human Neuron Core at Boston Children's Hospital following institutional guidelines and under institutional approvals (IRB #: P00016119). A clonal iPS cell line, MAN0855-0l #A (Coriell Institute #0R00007), was expanded from a female CDKL5 deficiency disorder patient carrying a heterozygous CDKL5 c.1412delA p.D471fs mutation on the X chromosome (Chen et al., 2021). MAN0855-01 #A was previously verified to express the mutant CDKL5 transcript by Sanger sequencing of cDNA. The MAN0855-01 #A iPS cell line was cultured in StemFlex medium (Thermo Fisher Scientific) on Geltrex (Thermo Fisher Scientific) diluted 1:50 in DMEM/F12 (Thermo Fisher Scientific) and coated according to the manufacturer's protocol. For regular maintenance. iPS cell colonies were clump-passaged using Gentle Cell Dissociation Reagent (STEMCELL Technologies) at 80% confluency every 5-7 days.

### Method Details

### General Methods and Molecular Cloning

Lentiviral transfer plasmids and plasmids for mammalian expression of prime editors and other proteins were cloned using uracil excision (USER) assembly (Cavaleiro et al., 2015). Briefly, DNA fragments were amplified with deoxyuracil-containing primers (Integrated DNA Technologies) using the uracil tolerant Phusion U Green Multiplex PCR Master Mix (Thermo Fisher Scientific). Deoxyuracil-incorporated DNA fragments were assembled with USER enzyme (New England BioLabs) and DpnI (New England BioLabs) according to the manufacturer's protocol using junctions with a melting temperature of 42-60° C., followed by transformation into cells. All prime editor constructs were cloned into the pCMV-PE2 vector backbone (Anzalone et al., 2019) (Addgene #132775) under constitutive expression from a CMV promoter. All prime editor constructs also contained the following mutations within the MMLV RT: D200N, T306K, W313F, T330P, and L603W. All DNA repair protein and RFP expression constructs were cloned into vectors under constitutive expression from an EFla promoter. Human MSH2, MSH6, PMS2, and MLH1 sequences were subcloned from the plasmids pFB1_hMSH2 (Addgene #129423), pFB1_hMSH6 (Addgene #129424), pFB1_PMS2 (Addgene #129425), and pFB1_MLH1 (Addgene #129426) (Geng et al., 2011). Human CDKN1A sequence was subcloned from the plasmid Flag p21 WT (Addgene #16240) (Zhou et al., 2001). Codon-optimized MLH1 sequences for human cell and mouse cell expression were designed using GenSmart Codon Optimization (Genscript) and ordered as gBlock gene fragments (Integrated DNA Technologies).

Plasmids for mammalian expression of pegRNAs or sgRNAs were cloned using Golden Gate assembly (Engler et al., 2008) as previously described (Anzalone et al., 2019). Briefly, a guide RNA vector backbone for human U6 promoter expression was digested overnight with BsaI-HFv2 (New England BioLabs) according to the manufacturer's protocol, and linearized product was purified by electrophoresis with a 1% agarose gel using the QIAquick Gel Extraction Kit (QIAGEN). Oligonucleotides (Integrated DNA Technologies) for the spacer sequence, guide RNA scaffold, and 3′ extension were annealed, assembled with linearized U6 backbone DNA using T4 DNA ligase (New England BioLabs) according to the manufacturer's protocol, and transformed into cells. Only guide RNA scaffold oligonucleotides were purchased with 5′ phosphorylation modifications. Some plasmids encoding pegRNAs and epegRNAs were synthesized by Twist Bioscience. A list of pegRNAs and nicking sgRNAs used in this work is provided in Table 7.

Unless otherwise noted, assembled plasmids were transformed into One Shot Mach1 cells (Thermo Fisher Scientific) and grown on Luria-Bertani (LB) or 2×YT agar with 50 μg ml⁻¹ carbenicillin (Gold Biotechnology). Plasmid sequences were fully verified by Sanger sequencing (Quintara Biosciences), and bacteria containing verified plasmids were grown in 2×YT medium with 100 μg ml⁻¹ carbenicillin (Gold Biotechnology). Plasmid DNA were isolated using the QIAGEN Plasmid Plus Midi Kit or QIAGEN Plasmid Plus Maxi Kit with endotoxin removal and 2× the recommended amount of RNase A in Buffer PL. Some pegRNA and sgRNA plasmid DNA were isolated with the PureYield Plasmid Miniprep System (Promega Corporation) with endotoxin removal. Plasmid DNA purified using the PureYield Plasmid Miniprep System were only used for HEK293T and HeLa cell transfections. All plasmids were eluted in nuclease-free water (QIAGEN) and quantified using a NanoDrop One UV-Vis spectrophotometer (Thermo Fisher Scientific).

### Lentivirus Production for Generating Cell Lines

To package lentivirus for generating stable cell lines, HEK293T cells were seeded on 6-well plates (Corning) at 7.5×10⁵ cells per well in DMEM supplemented with 10% FBS. At 60% confluency 16 h after seeding, cells were transfected with 12 μL Lipofectamine 2000 (Thermo Fisher Scientific) according to the manufacturer's protocol and 1.33 μg lentiviral transfer plasmid, 0.67 μg pMD2.G (Addgene #12259), and 1 μg psPAX2 (Addgene #12260). 6 h after transfection, media was exchanged with DMEM supplemented with 10% FBS. 48 h after transfection, viral supernatant was centrifuged at 3000 g for 15 min to remove cellular debris, filtered through a 0.45 μm PVDF filter (Corning), and stored at −80° C.

Design and Construction of HEK293T Line with Integrated HBB Coding Region

A lentiviral transfer plasmid was designed and cloned to contain the human HBB coding region (CDS) and a PuroR-T2A-BFP marker under expression from an EFla promoter (pEF1α). Lentivirus carrying this cassette were produced from HEK293T cells as described above. To stably integrate the HBB CDS, 6×10⁵ HEK293T cells were infected with lentivirus in 6-well plates (Corning) with DMEM supplemented with 10% FBS and 10 μg mL¹ polybrene (Sigma-Aldrich). BFP fluorescence was monitored daily using a CytoFLEX S Flow Cytometer (Beckman Coulter) to ensure an MOI of 0.1 and single copy integration. Following infection for 2 days, HEK293T cells were selected in 2 μg μL⁻¹ puromycin (Thermo Fisher Scientific) for 3 days and stable transduction was confirmed by measuring BFP fluorescence. The resulting cell line was used to optimize pegRNAs for prime editing at an integrated HBB site with SaPE2. To measure editing, a 214-bp amplicon of the integrated HBB CDS locus was PCR amplified. Amplification of the genomic HBB locus with these primers yields a differently sized 1064-bp amplicon.

Design and Construction of HeLa Line with CRISPRi sgRNA and Prime Edit Target

The lentiviral transfer plasmid backbone for prime editing Repair-seq screens (pPC1000) was designed and cloned to contain a universal prime edit site and express a control S. pyogenes sgRNA for CRISPRi targeting. The prime edit site consisted of an HBB target protospacer for Sa-pegRNA that is flanked by two complementary-strand Sa-sgRNA protospacers derived from the Saccharomyces cerevisiae genome. These protospacers were situated such that SaPE2-sgRNA complexes nick 50-bp upstream and 50-bp downstream of the nick formed by SaPE2-pegRNA. This 234-bp edit site was positioned adjacently to the sequence of an EGFP-targeting control S. pyogenes sgRNA under expression from a mouse U6 promoter such that sgRNA and edit site could be amplified by PCR in the same 453-bp amplicon. pPC1000 also contained an pEF1α-PuroR-T2A-BFP selection marker.

Lentivirus encoding the pPC1000 cassette were produced from HEK293T cells as described above. For stable integration, 2.5×10⁵ HeLa dCas9-BFP-KRAB cells were infected with pPC1000 lentivirus in 6-well plates (Corning) with DMEM supplemented with 10% FBS and 10 μg mL⁻¹ polybrene (Sigma-Aldrich). BFP fluorescence was monitored with a CytoFLEX S Flow Cytometer (Beckman Coulter) to ensure an MOI of 0.1 and single copy integration. Following 2 days of infection, cells were selected in 2 μg μL⁻¹ puromycin (Thermo Fisher Scientific) for 3 days and stable transduction was confirmed by measuring BFP fluorescence. The resulting HeLa dCas9-BFP-KRAB cell line with integrated pPC1000 sequence was used to pilot prime editing conditions, Sa-pegRNAs, and Sa-sgRNAs for Repair-seq screens.

### Transfection of HEK293T, HeLa, HCT116, and N2A Cells

Unless otherwise noted, HEK293T cells were seeded on 96-well plates (Corning) at 1.6-1.8×10⁴ cells per well in DMEM plus GlutaMAX supplemented with 10% FBS. Between 16 and 24 h after seeding, cells were transfected at 60-80% confluency with 0.5 μL Lipofectamine 2000 (Thermo Fisher Scientific) according to the manufacturer's protocol and 200 ng prime editor plasmid, 66 ng pegRNA plasmid, 22 ng sgRNA plasmid (when required), and 100 ng for RFP or MMR protein expression (when required).

For arrayed experiments, HeLa dCas9-BFP-KRAB and HeLa cells were seeded on 96-well plates (Corning) at 8×10³ cells per well in DMEM plus GlutaMAX supplemented with 10% FBS. Between 16 and 24 h after seeding, cells were transfected at 60-80% confluency with 0.3 μL TransIT-HeLa reagent (Mirus Bio) according to the manufacturer's protocol and 56.25 ng prime editor plasmid bearing a P2A-BlastR selection marker, 18.75 ng pegRNA plasmid, 6.25 sgRNA plasmid (when required), and 28.1 ng human codon-optimized MLH1dn plasmid (when required). 24 h following transfection, 10 ng μL⁻¹ blasticidin (Thermo Fisher Scientific) was added to each well to select for cells expressing prime editor.

HCT116 cells were seeded on 96-well plates (Corning) at 1.6×10⁴ cells per well in DMEM plus GlutaMAX supplemented with 10% FBS. Between 16 and 20 h after seeding, cells were transfected at 60-80% confluency with 0.5 μL Lipofectamine 3000 plus 0.8 μL P3000 reagent (Thermo Fisher Scientific) according to the manufacturer's protocol and 200 ng prime editor plasmid bearing a P2A-BlastR selection marker, 66 ng pegRNA plasmid, 22 ng sgRNA plasmid (when required), and 100 ng MLH1dn plasmid (when required). The day after transfection, media was replaced with fresh DMEM plus GlutaMAX supplemented with 10% FBS and 10 ng μL⁻¹ blasticidin (Thermo Fisher Scientific) to select for cells expressing prime editor.

N2A cells were seeded on 96-well plates (Corning) at 1.6×10⁴ cells per well in DMEM plus GlutaMAX supplemented with 10% FBS. Between 16 and 20 h after seeding, cells were transfected at 60-80% confluency with 0.5 μL Lipofectamine 2000 (Thermo Fisher Scientific) according to the manufacturer's protocol and 175 ng prime editor plasmid, 50 ng pegRNA plasmid, 20 ng sgRNA plasmid (when required), and 87.5 ng plasmid encoding human codon-optimized hMLH1dn or mouse codon-optimized mMLH1dn when required. Genomic DNA was extracted 72 h following transfection.

### Electroporation of HAP1, K562, and U2OS Cels

HAP1 cells were nucleofected using the SE Cell Line 4D-Nucleofector X Kit S (Lonza) according to the manufacturer's protocol with 4×10⁵ cells (program DZ-113), 300 ng PE2-P2A-BSD, 100 ng pegRNA plasmid, and 33 ng sgRNA plasmid (when required). After nucleofection, cells were cultured in 48-well plates (Corning) with IMDM plus GlutaMAX supplemented with 10% FBS. The day after nucleofection, media was replaced with fresh IMDM plus GlutaMAX supplemented with 10% FBS and 10 ng μL⁻¹ blasticidin (Thermo Fisher Scientific) to select for cells expressing prime editor.

K562 cells were nucleofected using the SF Cell Line 4D-Nucleofector X Kit S (Lonza) according to the manufacturer's protocol with 5×10⁵ cells (program FF-120), 800 ng prime editor plasmid, 200 ng pegRNA plasmid, 83 ng sgRNA plasmid (when required), and 400 ng MLH1dn plasmid (when required). After nucleofection, cells were cultured in 6-well plates (Corning) with RPMI 1640 medium supplemented with 10% FBS and 292 μg mL⁻¹ L-Glutamine (Thermo Fisher Scientific).

U2OS cells were nucleofected using the SE Cell Line 4D-Nucleofector X Kit S (Lonza) according to the manufacturer's protocol with 2×10⁵ cells (program DN-100), 1600 ng PE2 or PE2-P2A-MLH1dn plasmid, 400 ng pegRNA plasmid, and 166 ng sgRNA plasmid (when required). After nucleofection, cells were cultured in 12-well or 24-well plates (Corning) with McCoy's 5A medium supplemented with 10% FBS.

### Genomic DNA Extraction

Unless otherwise noted, HEK293T, HeLa dCas9-BFP-KRAB, HeLa, HCT116, N2A, HAP1, K562, and U2OS cells were cultured for 72 h after transfection or nucleofection before genomic DNA was isolated. Cells were washed once with PBS (Thermo Fisher Scientific) and lysed with gDNA lysis buffer (10 mM Tris-HCl, pH 8.0; 0.05% SDS; 800 units μL⁻¹ proteinase K (New England BioLabs)) at 37° C. for 1.5-2 h. followed by enzyme inactivation at 80° C. for 30 min.

### High-Throughput Amplicon Sequencing of Genomic DNA Samples

To assess gene editing, loci were amplified from genomic DNA samples via two rounds of PCR and deep sequenced. Briefly, an initial PCR step (PCR1) amplified the genomic sequence of interest using primers (Integrated DNA Technologies) containing Illumina forward and reverse adapters. Each 20 μL PCR 1 reaction was performed with 500 nM of each primer, 0.8 to 1.0 μL genomic DNA, 1×SYBR Green (Thermo Fisher Scientific), and 10 μL Q5 High-Fidelity 2× Master Mix (New England BioLabs) on a CFX96 Touch Real-Time PCR Detection System (Bio-Rad Laboratories) with the following thermocycling conditions: 98° C. for 2 min, 29-31 cycles of [98° C. for 10 s, 61° C. for 20 s, and 72° C. for 30 s], followed by 72° C. for 2 min. PCR 1 reactions were monitored with SYBR Green fluorescence to avoid over-amplification. A list of primers used for PCR1 reactions is provided in Table 8.

TABLE 8
Primers Used for PCR1 Reactions

| | | SEQ ID |
|---|---|---|
| Primer description | Primer sequence (5′-3′) | NO: |
| Lentiviral HBB CDS site-PCR1 | ACACTCTTTCCCTACACGACGCTCTTCCGATCTAAATT | 250 |
| forward | GGACAGCAAGAAAGCGAGCTTAG | |
| Lentiviral HBB CDS site-PCR1 | TGGAGTTCAGACGTGTGCTCTTCCGATCTCTTTGCCAC | 251 |
| reverse | ACTGAGTGAGCTG | |
| Lentiviral pPC1000 edit site-PCR1 | ACACTCTTTCCCTACACGACGCTCTTCCGATCTNNNNG | 252 |
| forward | CCTTTAGTGATGGCCTGGCTCACCTG | |
| Lentiviral pPC1000 edit site-PCR1 | TGGAGTTCAGACGTGTGCTCTTCCGATCTTAGGCGGCC | 253 |
| reverse | GCTGCACGTAG | |
| Lentiviral pPC1000 CRISPRi sgRNA | ACACTCTTTCCCTACACGACGCTCTTCCGATCTATCCC | 254 |
| and edit site | TTGGAGAACCACCTTGTTG | |
| (for Repair-seq screens)- | | |
| PCR1 forward | | |
| Lentiviral pPC1000 CRISPRi sgRNA | TGGAGTTCAGACGTGTGCTCTTCCGATCTCAACCAAC | 255 |
| and edit site | GCTATCGGCATGG | |
| (for Repair-seq screens)- | | |
| PCR1 reverse | | |
| HEK3 locus-PCR1 forward | ACACTCTTTCCCTACACGACGCTCTTCCGATCTNNNNC | 256 |
| | AGGGAAACGCCCATGCAATTAG | |
| HEK3 locus-PCR1 reverse | TGGAGTTCAGACGTGTGCTCTTCCGATCTCTCTGTTGA | 257 |
| | GCTCGACCCTGAA | |
| EMX1 locus-PCR1 forward | ACACTCTTTCCCTACACGACGCTCTTCCGATCTNNNNC | 258 |
| | AGCTCAGCCTGAGTGTTGA | |
| EMX1 locus-PCR1 reverse | TGGAGTTCAGACGTGTGCTCTTCCGATCTCTCGTGGGT | 259 |
| | TIGTGGTTGC | |
| RNF2 locus-PCR1 forward | ACACTCTTTCCCTACACGACGCTCTTCCGATCTNNNNT | 260 |
| | TAGCCAACATACAGAAGTCAGG | |
| RNF2 locus-PCR1 reverse | TGGAGTTCAGACGTGTGCTCTTCCGATCTAGACCATA | 261 |
| | GCACTTCCCTTCC | |
| FANCF locus-PCR1 forward | ACACTCTTTCCCTACACGACGCTCTTCCGATCINNNNC | 262 |
| | GATGGATGTGGCGCAGGTAG | |
| FANCF locus-PCR1 reverse | TGGAGTTCAGACGTGTGCTCTTCCGATCTAGACGCTG | 263 |
| | GGAGATTGACATGC | |
| RUNX1 locus-PCR1 forward | ACACTCTTTCCCTACACGACGCTCTTCCGATCTNNNNT | 264 |
| | CACAAACAAGACAGGGAACTG | |
| RUNX1 locus-PCR1 reverse | TGGAGTTCAGACGTGTGCTCTTCCGATCTAGATGTAG | 265 |
| | GGCTAGAGGGGTG | |
| DNMT1 locus-PCR1 forward | ACACTCTTTCCCTACACGACGCTCTTCCGATCTNNNNC | 266 |
| | ACAACAGCTTCATGTCAGCC | |
| DNMT1 locus-PCR1 reverse | TGGAGTTCAGACGTGTGCTCTTCCGATCTACGTTAATG | 267 |
| | TTTCCTGATGGTCC | |
| VEGFA locus-PCR1 forward | ACACTCTTTCCCTACACGACGCTCTTCCGATCTNNNNA | 268 |
| | CTTGGTGCCAAATTCTTCTCC | |
| VEGFA locus-PCR1 reverse | TGGAGTTCAGACGTGTGCTCTTCCGATCTAAAGAGGG | 269 |
| | AATGGGCTTTGGA | |
| HEK4 locus-PCR1 forward | ACACTCTTTCCCTACACGACGCTCTTCCGATCTNNNNG | 270 |
| | AACCCAGGTAGCCAGAGAC | |
| HEK4 locus-PCR1 reverse | TGGAGTTCAGACGTGTGCTCTTCCGATCTTCCTTTCAA | 271 |
| | CCCGAACGGAG | |
| HBB locus-PCR1 forward | ACACTCTTTCCCTACACGACGCTCTTCCGATCTNNNNG | 252 |
| | CCTTTAGTGATGGCCTGGCTCACCTG | |
| HBB locus-PCR1 reverse | TGGAGTTCAGACGTGTGCTCTTCCGATCTGTCTTCTCT | 272 |
| | GTCTCCACATGCC | |
| PRNP locus-PCR1 forward | ACACTCTTTCCCTACACGACGCTCTTCCGATCTNNNNG | 273 |
| | TCAGTGGAACAAGCCGAGT | |
| PRNP locus-PCR1 reverse | TGGAGTTCAGACGTGTGCTCTTCCGATCTACTTGGTTG | 274 |
| | GGGTAACGGTG | |
| CDKL5 locus-PCR1 forward | ACACTCTTTCCCTACACGACGCTCTTCCGATCINNNNC | 275 |
| | TTCAGAAGGCCCAGGGACAAAG | |
| CDKL5 locus-PCR1 reverse | TGGAGTTCAGACGTGTGCTCTTCCGATCTCTGGTACTG | 276 |
| | GGCTCCGCAATTT | |
| CXCR4 locus-PCR1 forward | ACACTCTTTCCCTACACGACGCTCTTCCGATCTNNNNT | 277 |
| | CTTGAGGGCCTTGCGCTTC | |
| CXCR4 locus-PCR1 reverse | TGGAGTTCAGACGTGTGCTCTTCCGATCTCCTTCATCA | 278 |
| | GTCTGGACCGCTA | |
| IL2RB locus-PCR1 forward | ACACTCTTTCCCTACACGACGCTCTTCCGATCTNNNNG | 279 |
| | GACCAGGACAGGAAGGAGGAA | |
| IL2RB locus-PCR1 reverse | TGGAGTTCAGACGTGTGCTCTTCCGATCTGTGGCAGGT | 280 |
| | ACAAAGTGGGAGG | |
| HBG1/2 locus-PCR1 forward | ACACTCTTTCCCTACACGACGCTCTTCCGATCTNNNNT | 281 |
| | CTTCATCCCTAGCCAGCCGC | |
| HBG1/2 locus-PCR1 reverse | TGGAGTTCAGACGTGTGCTCTTCCGATCTAACGGCTG | 282 |
| | ACAAAAGAAGTCCTGG | |
| mouse Dnmt1 locus-PCR1 forward | ACACTCTTTCCCTACACGACGCTCTTCCGATCTNNNNC | 283 |
| | CTTCGGGCATAGCATGG | |
| mouse Damt1 locus-PCR1 reverse | TGGAGTTCAGACGTGTGCTCTTCCGATCTCCTCGGCAT | 284 |
| | CGGTCC | |
| mouse Chd2 locus-PCR1 forward | ACACTCTTTCCCTACACGACGCTCTTCCGATCTNNNNA | 285 |
| | GTCAGTGCCTCACCTCTCACAC | |
| mouse Chd2 locus-PCR1 reverse | TGGAGTTCAGACGTGTGCTCTTCCGATCTCTTGCAGAT | 286 |
| | CGAGGAGACTOGCA | |
| mouse Col12a1 locus-PCR1 forward | ACACTCTTTCCCTACACGACGCTCTTCCGATCTNNNNT | 287 |
| | GAAGTCATGTGCGGTCTGGTCA | |
| mouse Col12a1 locus-PCR1 reverse | TGGAGTTCAGACGTGTGCTCTTCCGATCTAGCTCACAG | 288 |
| | CCTCATGAAGGGA | |
| mouse Ctnnb1 locus-PCR1 forward | ACACTCTTTCCCTACACGACGCTCTTCCGATCTNNNNA | 289 |
| | CCAGCTACTTGCTCTTGCGTG | |
| mouse Ctnnbl locus-PCR1 reverse | TGGAGTTCAGACGTGTGCTCTTCCGATCTCGTAGATGG | 290 |
| | CTTCTTCAGGTAGCA | |
| HEK3 OT1 locus-PCR1 forward | ACACTCTTTCCCTACACGACGCTCTTCCGATCTNNNNT | 291 |
| | CCCCTGTTGACCTGGAGAA | |
| HEK3 OT1 locus-PCR1 reverse | TGGAGTTCAGACGTGTGCTCTTCCGATCTCACTGTACT | 292 |
| | TGCCCTGACCA | |
| HEK3 OT2 locus-PCR1 forward | ACACTCTTTCCCTACACGACGCTCTTCCGATCTNNNNT | 293 |
| | TGGTGTTGACAGGGAGCAA | |
| HEK3 OT2 locus-PCR1 reverse | TGGAGTTCAGACGTGTGCTCTTCCGATCTCTGAGATGT | 294 |
| | GGGCAGAAGGG | |
| HEK3 OT3 locus-PCR1 forward | ACACTCTTTCCCTACACGACGCTCTTCCGATCTNNNNT | 295 |
| | GAGAGGGAACAGAAGGGCT | |
| HEK3 OT3 locus-PCR1 reverse | TGGAGTTCAGACGTGTGCTCTTCCGATCTGTCCAAAG | 296 |
| | GCCCAAGAACCT | |
| HEK3 OT4 locus-PCR1 forward | ACACTCTTTCCCTACACGACGCTCTTCCGATCTNNNNT | 297 |
| | CCTAGCACTTTGGAAGGTCG | |
| HEK3 OT4 locus-PCR1 reverse | TGGAGTTCAGACGTGTGCTCTTCCGATCTGCTCATCTT | 298 |
| | AATCTGCTCAGCC | |
| FANCF OT1 locus-PCR1 forward | ACACTCTTTCCCTACACGACGCTCTTCCGATCTNNNNG | 299 |
| | CGGGCAGTGGCGTCTTAGTCG | |
| FANCF OT1 locus-PCR1 reverse | TGGAGTTCAGACGTGTGCTCTTCCGATCTCCCTGGGTT | 300 |
| | TGGTTGGCTGCTC | |
| FANCF OT2 locus-PCR1 forward | ACACTCTTTCCCTACACGACGCTCTTCCGATCTNNNNC | 301 |
| | TCCTTGCCGCCCAGCCGGTC | |
| FANCF OT2 locus-PCR1 reverse | TGGAGTTCAGACGTGTGCTCTTCCGATCTCACTGGGG | 302 |
| | AAGAGGCGAGGACAC | |
| FANCF OT3 locus-PCR1 forward | ACACTCTTTCCCTACACGACGCTCTTCCGATCTNNNNC | 303 |
| | CAGTGTTTCCCATCCCCAACAC | |
| FANCF OT3 locus-PCR1 reverse | TGGAGTTCAGACGTGTGCTCTTCCGATCTGAATGGATC | 304 |
| | CCCCCCTAGAGCTC | |
| FANCF OT4 locus-PCR1 forward | ACACTCTTTCCCTACACGACGCTCTTCCGATCTNNNNC | 305 |
| | AGGCCCACAGOTCCTTCTGGA | |
| FANCF OT4 locus-PCR1 reverse | TGGAGTTCAGACGTGTGCTCTTCCGATCTCCACACGG | 1306 |
| | AAGGCTGACCACG | |
| EMX1 OT1 locus-PCR1 forward | ACACTCTTTCCCTACACGACGCTCTTCCGATCTNNNNG | 307 |
| | TGGGGAGATTTGCATCTGTGGAGG | |
| EMX1 OT1 locus-PCR1 reverse | TGGAGTTCAGACGTGTGCTCTTCCGATCTGCTTTTATA | 308 |
| | CCATCTTGGGGTTACAG | |
| EMX1 OT2 locus-PCR1 forward | ACACTCTTTCCCTACACGACGCTCTTCCGATCTNNNNC | 309 |
| | AATGTGCTTCAACCCATCACGGC | |
| EMX1 OT2 locus-PCR1 reverse | TGGAGTTCAGACGTGTGCTCTTCCGATCTCCATGAATT | 310 |
| | TGTGATGGATGCAGTCTG | |
| EMX1 OT3 locus-PCR1 forward | ACACTCTTTCCCTACACGACGCTCTTCCGATCTNNNNG | 311 |
| | AGAAGGAGGTGCAGGAGCTAGAC | |
| EMX1 OT3 locus-PCR1 reverse | TGGAGTTCAGACGTGTGCTCTTCCGATCTCATCCCGAC | 312 |
| | CTTCATCCCTCCTGG | |
| EMX1 OT4 locus-PCR1 forward | ACACTCTTTCCCTACACGACGCTCTTCCGATCTNNNNA | 313 |
| | CAGGTGAATAACCGGTCGCCA | |
| EMXI OT4 locus-PCR1 reverse | TGGAGTTCAGACGTGTGCTCTTCCGATCTTTTATCAAA | 314 |
| | CAAGGTGCAGATACAGCAA | |
| HEK4 OT1 locus-PCR1 forward | ACACTCTTTCCCTACACGACGCTCTTCCGATCTNNNNG | 315 |
| | GCATGGCTTCTGAGACTCA | |
| HEK4 OT1 locus-PCR1 reverse | TGGAGTTCAGACGTGTGCTCTTCCGATCTGTCTCCCTT | 316 |
| | GCACTCCCTGTCTTT | |
| HEK4 OT2 locus-PCR1 forward | ACACTCTTTCCCTACACGACGCTCTTCCGATCTNNNNT | 317 |
| | TTGGCAATGGAGGCATTGG | |
| HEK4 OT2 locus-PCR1 reverse | TGGAGTTCAGACGTGTGCTCTTCCGATCTGAAGAGGC | 318 |
| | TGCCCATGAGAG | |
| HEK4 OT3 locus-PCR1 forward | ACACTCTTTCCCTACACGACGCTCTTCCGATCTNNNNG | 319 |
| | GTCTGAGGCTCGAATCCTG | |
| HEK4 OT3 locus-PCR1 reverse | TGGAGTTCAGACGTGTGCTCTTCCGATCTCTGTGGCCT | 320 |
| | CCATATCCCTG | |
| HEK4 OT4 locus-PCR1 forward | ACACTCTTTCCCTACACGACGCTCTTCCGATCTNNNNT | 321 |
| | TTCCACCAGAACTCAGCCC | |
| HEK4 OT4 locus-PCR1 reverse | TGGAGTTCAGACGTGTGCTCTTCCGATCTCCTCGGTTC | 322 |
| | CTCCACAACAC | |
| Bat-25 microsatellite-PCR1 forward | ACACTCTTTCCCTACACGACGCTCTTCCGATCTGGAGG | 323 |
| | ATGACGAGTTGGCCCTAGAC | |
| Bat-25 microsatellite-PCR1 reverse | TGGAGTTCAGACGTGTGCTCTTCCGATCTCCAAAGAG | 324 |
| | ACAGCAGTTGGAACATGA | |
| Bat-26 microsatellite-PCR1 forward | ACACTCTTTCCCTACACGACGCTCTTCCGATCTAGTGG | 325 |
| | AGTGGAGGAGGGGAGAGAAA | |
| Bat-26 microsatellite-PCR1 reverse | TGGAGTTCAGACGTGTGCTCTTCCGATCTTTGCAGTTT | 326 |
| | CATCACTGTCTGCGGT | |
| MONO-27 microsatellite-PCR1 | ACACTCTTTCCCTACACGACGCTCTTCCGATCTCTACT | 327 |
| forward | GTCCTACTGTGCCTGGCTCC | |
| MONO-27 microsatellite-PCR1 | TGGAGTTCAGACGTGTGCTCTTCCGATCTCAGCCTGGG | 328 |
| reverse | CAAGATAATGAGACCC | |
| NR-21 microsatellite-PCR1 forward | ACACTCTTTCCCTACACGACGCTCTTCCGATCTCTGGT | 329 |
| | GCACAGAGCAGAACCATCCT | |
| NR-21 microsatellite-PCR1 reverse | TGGAGTTCAGACGTGTGCTCTTCCGATCTGCAACCTCA | 330 |
| | AAAGCTGCCTCCCTTT | |
| NR-24 microsatellite-PCR1 forward | ACACTCTTTCCCTACACGACGCTCTTCCGATCTCTGTA | 331 |
| | GTCCCAGCTATTCGGGAGGC | |
| NR-24 microsatellite-PCR1 reverse | TGGAGTTCAGACGTGTGCTCTTCCGATCTAAATGACCC | 332 |
| | CTTCCTGCCCATCACT | |
| HSPH1-T17 microsatellite-PCR1 | ACACTCTTTCCCTACACGACGCTCTTCCGATCTTGGAA | 333 |
| forward | AAGGAACTGCATCTGTGACGG | |
| HSPH1-T17 microsatellite-PCR1 | TGGAGTTCAGACGTGTGCTCTTCCGATCTTTTTCCTAA | 334 |
| reverse | TCCCCTGTGAAACCTGT | |
| EWSR1 microsatellite-PCR1 | ACACTCTTTCCCTACACGACGCTCTTCCGATCTAACAA | 335 |
| forward | TGTTCATGGTTGTGATGT | |
| EWSR1 microsatellite-PCR1 reverse | TGGAGTTCAGACGTGTGCTCTTCCGATCTCTGGATCTC | 336 |
| | CCCTTGAACCTTTGGA | |
| MSI-01 microsatellite-PCR1 forward | ACACTCTTTCCCTACACGACGCTCTTCCGATCTTTGAT | 337 |
| | GTCCTGCGTCTAGGGTCTGC | |
| MSI-01 microsatellite-PCR1 reverse | TGGAGTTCAGACGTGTGCTCTTCCGATCTGACTGGAG | 338 |
| | CCTTGGACAGGTTGAGA | |
| MSI-03 microsatellite-PCR1 forward | ACACTCTTTCCCTACACGACGCTCTTCCGATCTGOCAC | 339 |
| | TGCTATTTGAAAGAGTTGCTC | |
| MSI-03 microsatellite-PCR1 reverse | TGGAGTTCAGACGTGTGCTCTTCCGATCTCACACCTGG | 340 |
| | CTAAATGCTCGGATT | |
| MSI-04 microsatellite-PCR1 forward | ACACTCTTTCCCTACACGACGCTCTTCCGATCTTCCAA | 341 |
| | GATTCCTTCCCTGGCCACTC | |
| MSI-04 microsatellite-PCR1 reverse | TGGAGTTCAGACGTGTGCTCTTCCGATCTACTGTCTGT | 342 |
| | AGTCCTGGCTTCGTGG | |
| MSI-06 microsatellite-PCR1 forward | ACACTCTTTCCCTACACGACGCTCTTCCGATCTAGCAG | 343 |
| | CAAACTGAACAGGTCACCAAC | |
| MSI-06 microsatellite-PCR1 reverse | TGGAGTTCAGACGTGTGCTCTTCCGATCTGCTACACTG | 344 |
| | TGGCATCAGCACATATC | |
| MSI-07 microsatellite-PCR1 forward | ACACTCTTTCCCTACACGACGCTCTTCCGATCTGCTGA | 345 |
| | AAGCAACCTAAGCTGTGGTGA | |
| MSI-07 microsatellite-PCR1 reverse | TGGAGTTCAGACGTGTGCTCTTCCGATCTTGCTATAAG | 346 |
| | AGCTGAGCAGACGACA | |
| MSI-08 microsatellite-PCR1 forward | ACACTCTTTCCCTACACGACGCTCTTCCGATCTCCAGC | 347 |
| | CCCCATGTACACTGTAGTOG | |
| MSI-08 microsatellite-PCR1 reverse | TGGAGTTCAGACGTGTGCTCTTCCGATCTCCCACCCCA | 348 |
| | AGGCCAAAATCAGTAA | |
| MSI-09 microsatellite-PCR1 forward | ACACTCTTTCCCTACACGACGCTCTTCCGATCTGTCTC | 349 |
| | GGCTACTTGGGAGGCTTAGG | |
| MSI-09 microsatellite-PCR1 reverse | TGGAGTTCAGACGTGTGCTCTTCCGATCTCCGACTAAA | 350 |
| | GAGGTCATTCACTTGT | |
| MSI-11 microsatellite-PCR1 forward | ACACTCTTTCCCTACACGACGCTCTTCCGATCTAGCAT | 351 |
| | GTTTGCAGCCTTCTTCTGGA | |
| MSI-11 microsatellite-PCR1 reverse | TGGAGTTCAGACGTGTGCTCTTCCGATCTAGAACTTCT | 352 |
| | CTCACAATGTAGCCCCT | |
| MSI-12 microsatellite-PCR1 forward | ACACTCTTTCCCTACACGACGCTCTTCCGATCTGCTGA | 353 |
| | GGCTAAACACTATCATGCCA | |
| MSI-12 microsatellite-PCR1 reverse | TGGAGTTCAGACGTGTGCTCTTCCGATCTCAGAGGTTG | 354 |
| | CAGTGAGCCGAGATTG | |
| MSI-13 microsatellite-PCR1 forward | ACACTCTTTCCCTACACGACGCTCTTCCGATCTACATC | 355 |
| | TTCAGGTCAGGAAAACAGCTCG | |
| MSI-13 microsatellite-PCR1 reverse | TGGAGTTCAGACGTGTGCTCTTCCGATCTTAATGACTT | 356 |
| | GGGCTTTGGAAGCAGC | |
| MSH2-RT-qPCR forward | CTTCGTGCGCTTCTTTCAGGG | 357 |
| MSH2-RT-qPCR reverse | GCAGATTCTTTGCTCCTGCCG | 358 |
| MSH6-RT-qPCR forward | CGCTGAGTGATGCCAACAAGG | 359 |
| MSH6-RT-qPCR reverse | AGCCCTCCGTTGAGGTTCTTC | 360 |
| MLHI-RT-qPCR forward | AAGACAATGGCACCGGGATCA | 361 |
| MLH1-RT-qPCR reverse | AAGCCTCACCTCGAAAGCCAT | 362 |
| PMS2-RT-qPCR forward | CTGGGCAGGTGGTACTGAGTC | 363 |
| PMS2-RT-qPCR reverse | TAGTGGCACCAGCATCCAGAC | 364 |
| ACTB-RT-PCR forward | GAGGCACTCTTCCAGCCTT | 365 |
| ACTB-RT-qPCR reverse | AAGGTAGTTTCGTGGATGCC | 366 |

The subsequent PCR step (PCR2) added unique i7 and i5 Illumina barcode combinations to both ends of the PCR1 DNA fragment to enable sample demultiplexing. Each 12.5 μL PCR2 reaction was performed with 500 nM of each barcoding primer, 0.5 μL PCR 1 product, and 6.25 μL Phusion U Green Multiplex PCR Master Mix (Thermo Fisher Scientific) with the following thermocycling conditions: 98° C. for 2 min, 9 cycles of [98° C. for 15 s, 61° C. for 20 s, and 72° C. for 30 s], followed by 72° C. for 2 min. PCR2 products were pooled by common amplicons, separated by electrophoresis on a 1% agarose gel, purified using the QIAquick Gel Extraction Kit (QIAGEN), and eluted in nuclease-free water. DNA amplicon libraries were quantified with a Qubit 3.0 Fluorometer (Thermo Fisher Scientific), then sequenced using the MiSeq Reagent Kit v2 or MiSeq Reagent Micro Kit v2 (Illumina), with 280-300 single-read cycles. A list of FASTQ sequencing files generated in this work is provided in Table 7.

### Quantification of Amplicon Sequencing Data

All arrayed prime editing experiments from FIGS. 42A-54B were analyzed as follows. Sequencing reads were demultiplexed using MiSeq Reporter (Illumina). Amplicon sequences were aligned to a reference sequence with CRISPResso2 (Clement et al., 2019) in standard mode using the parameters “−q 30” and “−discard_indel_reads TRUE”. For each amplicon, the CRISPResso2 quantification window was positioned to include the entire sequence between pegRNA- and sgRNA-directed Cas9 cut sites, as well as an additional >10-nt beyond both cut sites. For each amplicon, the same quantification window was used for PE2, PE3, PE4, and PE5 conditions, regardless of whether a nicking sgRNA is used. All prime editing efficiencies describe percentage of (number of reads with the intended edit that do not contain indels)/(number of reads that align to the amplicon). Single-base substitution prime editing frequencies were quantified as: (frequency of intended base substitution in reference-aligned, non-discarded reads)×(number of reference-aligned, non-discarded reads)/(number of reference-aligned reads). For all other prime edits (insertion, deletion, contiguous substitutions, combinations of edits). CRISPResso2 was run in HDR mode with all the same parameters described above and using the intended editing outcome as the expected allele (−e). Frequencies for these edits were quantified as: (number of HDR-aligned reads)/(number of reference-aligned reads). All indel frequencies were quantified as: (number of indel-containing reads)/(number of reference-aligned reads).

### CRISPRi Library Cloning and Lentivirus Production

An oligonucleotide library of CRISPRi sgRNAs, oBA697, was designed to contain 60 non-targeting control sgRNAs and 1.513 sgRNAs that target 477 DNA repair genes with three sgRNAs per gene (Hussmann et al., 2021). A list of targeted genes and sequences in the sgRNA library is provided in Table 5. The oBA697 oligonucleotide library was amplified by PCR using Phusion High-Fidelity DNA Polymerase (New England BioLabs) and purified with the NucleoSpin Gel and PCR Clean-up kit (Macherey-Nagel). The amplified CRISPRi library inserts and the pPC1000 lentiviral screen vector containing the pre-validated prime edit site were digested with BstXI and BlpI restriction endonucleases (Thermo Fisher Scientific), ligated with T4 ligase (New England BioLabs), then transformed into Stellar cells (Takara Bio). The pooled plasmid library was amplified in MegaX DH10B electrocompetent cells (Thermo Fisher Scientific). The pooled library sequences were further verified by sequencing on a MiSeq Reagent Kit v2 (Illumina).

To produce lentivirus with pPC1000 libraries, HEK293T cells were seeded in a 15 cm dish with DMEM supplemented with 10% FBS. One day after seeding, cells were transfected using 60 μL TransIT-LT1 reagent (Mirus Bio) with 15 μg pPC1000 plasmid library and 5 μg packaging plasmids for expression of HIV-1 gag/pol, rev, tat, and VSV-G envelope protein. 24 h after transfection, 40 μL ViralBoost reagent (ALSTEM) was added to each 15 cm dish. 48 h after transfection, viral supernatant was collected, filtered through a 0.45 μm PVDF filter (Corning), and stored at −80° C.

### Repair-Seq Screens in HeLa Cells

PE2, PE3+50, PE3-50 Repair-seq screens were performed in biological duplicate in HeLa cells with integrated dCas9-BFP-KRAB (Addgene #46911) (Gilbert et al., 2013). Lentivirus containing the CRISPRi library were produced from HEK293T cells as described above. HeLa CRISPRi cells expressing dCas9-KRAB were transduced with the lentiviral library at 0.1 MOI in DMEM supplemented with 10% FBS, 100 U mL¹ penicillin, 100 μg mL⁻¹ streptomycin (Thermo Fisher Scientific), and 8 μg mL⁻¹ polybrene (Sigma-Aldrich). 2 days after infection, HeLa CRISPRi cells were treated with 1 μg mL⁻¹ puromycin (Thermo Fisher Scientific) to select for HeLa CRISPRi cells with integrated library members. 3 days after infection, an additional 2 μg mL⁻¹ puromycin was added to cells. Throughout lentiviral transduction and selection steps, cells were analyzed for BFP fluorescence on a BD LSRII flow cytometer to ensure a MOI of 0.1 and completed selection. Following 3 days of selection, media was changed to DMEM supplemented with 10% FBS, and HeLa CRISPRi cells were transfected at 50% confluency in 150 mm culture dishes (Corning) with 30 μg SaPE2-P2A-BSD plasmid, 10 μg Sa-pegRNA plasmid for installing a +6 G⋅C to C⋅G edit at the pre-validated edit site, and 3.3 μg Sa-sgRNA plasmid for +50 or −50 complementary-strand nicking (when required), using 140 μL TransIT-HeLa reagent (Mirus Bio) according to the manufacturer's protocol. For an unedited HeLa control condition, cells were transfected with only 30 μg SaPE2-P2A-BSD plasmid as described above. 24 h following transfection, cells were treated with 10 μg mL¹ blasticidin (Thermo Fisher Scientific) to select for expression of SaPE2 protein. 72 h after transfection, HeLa CRISPRi cells were washed with PBS (Thermo Fisher Scientific), resuspended using Trypsin and DMEM, and pelleted at 1000 g for 10 min. Finally, cells were washed once more with PBS, pelleted at 1000 g for 10 min, then stored at −80° C.

### Repair-Seq Screens in K562 Cells

PE2 and PE3+50 Repair-seq screens were performed in biological duplicate in K562 cells with integrated dCas9-BFP-KRAB (Addgene #46911) (Gilbert et al., 2014). Lentivirus containing the CRISPRi library were produced from HEK293T cells as described above. K562 CRISPRi cells expressing dCas9-KRAB were transduced with the lentiviral library at 0.2 MOI in RPMI supplemented with 10% FBS, 100 U mL⁻¹ penicillin, 100 μg mL⁻¹ streptomycin, 292 μg mL⁻¹ L-Glutamine, and 8 μg mL⁻¹ polybrene (Sigma-Aldrich) by centrifugation at 1000 g for 2 h at room temperature. 2 days post infection, K562 CRISPRi cells were treated with 3 μg mL⁻¹ puromycin (Thermo Fisher Scientific) to select for cells with integrated library members. After infection, the density of the K562 CRISPRi cells was maintained at approximately 5×10⁵ mL⁻¹ and the culture was replaced by fresh RPMI supplemented with 10% FBS. 100 U mL⁻¹ penicillin, 100 μg mL⁻¹ streptomycin, 292 μg mL⁻¹ L-Glutamine, and 3 μg mL⁻¹ puromycin 3 days and 5 days post infection. During media replacement, the cells were pelleted, washed with DPBS and resuspended in fresh media to remove dead cells. All centrifugations were performed at 150 g for 5 min in 50 mL canonical tubes to avoid cell loss. 6 days post infection, the culture was replaced twice by fresh RPMI supplemented with 10% FBS and 292 μg mL⁻¹ L-Glutamine (Thermo Fisher Scientific) to remove dead cells and antibiotics. Throughout lentiviral transduction and selection steps, cells were analyzed for BFP fluorescence on an Attune N×T flow cytometer to ensure an MOI of 0.2 and completed selection. 7 days post infection, the cells were nucleofected using the SE Cell Line 4D-Nucleofector X kit L (Lonza) with 1×10⁷ cells (program FF-120), 7.5 μg SaPE2 plasmid, 2.5 μg Sa-pegRNA plasmid for installing a +6 G⋅C to C⋅G edit at the pre-validated edit site, and 3.3 μg Sa-sgRNA plasmid for +50 complementary-strand nicking (for PE3+50 conditions). For an unedited K562 control condition, cells were mock electroporated without any DNA plasmid as described above. After nucleofection, the cells were seeded at a density of 5×10⁵ mL⁻¹ in RPMI supplemented with 10% FBS and 292 μg mL⁻¹ L-Glutamine. 48 h post nucleofection, the culture were pipetted up and down 5 times to prevent cells from clumping. 84 h post nucleofection, the cells were pelleted at 1000 g for 10 min, washed with DPBS, pelleted at 1000 g for 10 min, and then stored at −80° C.

### High-Throughput Sequencing of Repair-Seq Libraries

Genomic DNA from edited HeLa CRISPRi and K562 CRISPRi cells was extracted using NucleoSpin Blood XL Maxi kit (Machery-Nagel). The number of live cells from which genomic DNA was extracted for each Repair-seq condition is listed in Table 9.

TABLE 9
High-throughput Sequencing of Repair-seq Libraries

| PE repair- | Number of live cells | Number of |
|---|---|---|
| seq screen | harvested for genomic | sequencing |
| condition | DNA extraction (× 10e6) | reads |
| K562 PE2 rep1 | 83.72 | 118658517 |
| K562 PE2 rep2 | 118.69 | 188344211 |
| K562 PE3 + 50 rep1 | 70.213 | 230946050 |
| K562 PE3 + 50 rep2 | 90.965 | 206570163 |
| HeLa PE2 rep1 | 107.85 | 420685828 |
| HeLa PE2 rep2 | 73.5 | 171390008 |
| HeLa PE3 + 50 rep1 | 105.6 | 229364033 |
| HeLa PE3 + 50 rep2 | 95.4 | 290852565 |
| HeLa PE3-50 rep1 | 93.15 | 234577915 |
| HeLa PE3-50 rep2 | 96.9 | 150012222 |

The genomic DNA was used as template for the initial round of PCR (PCR1) to amplify the region of interest. For each sample, a 100 μL PCR1 reaction was performed with 1 μL of 100 μM of each primer for amplifying pPC1000 sgRNA and edit site (Table 8), 10 μg of genomic DNA as template, 50 μL of NEBNext Ultra II Q5 Master Mix (New England BioLabs), and molecular biology grade water (Corning) on a BioRad C1000 thermal cycler with the following thermocycler conditions: 98° C. for 30 s, 22 cycles of [98° C. for 10 s, 65° C. for 75 s], followed by 65° C. for 5 min. These amplification reactions were verified by TBE gel electrophoresis and ethidium bromide staining. The amplified products were purified using SPRIselect (Beckman Coulter) prior to the following round of PCR amplification (PCR2). Purified PCR 1 amplicons were quantified using a high sensitivity DNA chip (Agilent Technologies) on an Agilent 2100 Bioanalyzer. PCR2 enabled indexing of the samples by the addition of i7 and i5 Illumina barcodes. For each 50 μL indexing PCR2 reaction: 10 ng of PCR1 amplicon was used as template along with 25 μL of KAPA HiFi HotStart ReadyMix (Roche Molecular Systems), 3 μL of 10 μM of each barcoding primer with the following thermocycler conditions: 95° C. for 3 min, 8 cycles of [98° C. for 20 s, 65° C. for 15 s, 72° C. for 15 s], followed by 72° C. for 1 min. The reactions were verified by TBE gel electrophoresis and ethidium bromide staining. PCR2 products from four PCR2 products were purified using SPRIselect and quantified on an Agilent 2100 Bioanalyzer prior to pooling. Repair-seq libraries were sequenced with the NovaSeq 6000 S1 Reagent Kit v1.5 (Illumina) with two 8-nt index reads, 44 cycles for R 1 read, 263 cycles for R2 read. The number of sequencing reads acquired for each screen condition and replicate are listed in Table 9.

### Processing of Repair-Seq Screen Data

Repair-seq screen data was processed using a modified version of the analysis approach described in (Hussmann et al., 2021), with modifications made to accommodate the different library preparation strategy used in this Example (direction amplification of genomic DNA without ligation of UMIs before amplification) and the qualitatively different categories of repair outcomes empirically observed in prime editing data.

Briefly, sequencing data for a batch of screens consists of 4 reads per spot: 2 8-nt index reads, a 44-nt R1 read of the CRISPRi sgRNA, and a 263-nt R2 read of the repair outcome. Reads from a batch of screens are demultiplexed into individual screens based on index reads. Within each screen, reads are demultiplexed into sets representing outcomes from cells receiving each individual CRISPRi sgRNA by comparing R1 sequences to a table of expected CRISPRi sgRNAs, allowing up to one mismatch between observed and expected sequences. Because direct amplification without UMIs does not allow consensus error correction of multiple reads of each repair outcome, analysis must account for presence of errors in outcome sequences introduced by PCR or by sequencing to avoid interpreting such errors as genuine editing outcomes. As an initial triage, reads with less than 60% of base calls with a quality score greater than or equal to 30 were discarded.

To categorize a repair outcome sequencing read that passed this quality filter, the outcome was first locally aligned to the screen vector, the pegRNA sequence, the human genome (hg19) and the Bos taurus genome (bosTau7) to identify a comprehensive set of alignments between portions of the outcome sequence and any of these reference sequences. The set of local alignments identified was then pruned to a parsimonious set of alignments that explains as much of the read as possible using the minimum number of alignments using a greedy approach. The parsimonious alignments are then parsed through a decision tree that examines their configuration to assign the outcome to a category.

Outcomes were classified as unedited if they consisted of a single alignment to the screen vector that did not contain the programmed SNV or any indels, with the exception of deletions of 1-nt that did not fall within 5-nt of a programmed nick, which were considered possible sequencing or PCR errors and were disregarded. Outcomes were classified as deletions if they consisted of two alignments to the screen vector that collectively covered the entire read but omitted a segment of the screen vector. Outcomes were classified as tandem duplications if they consisted of two or more alignments to the screen vector that collectively covered the entire read such that the portions of the screen vector covered by any two consecutive alignments on the read overlapped. Outcomes were classified as joining of pegRNA sequence at an unintended location if the set of parsimonious alignments included an alignment to the pegRNA such that the primer binding site (PBS) of the pegRNA was aligned to the same part of the read as the PBS in a screen vector alignment but the reverse transcription template (RTT) of the pegRNA was not aligned to the same part of the read as the RTT in a screen vector alignment. Note that in some such cases, the sequence produced is also consistent in theory with a multi-stage editing event consisting of an initial deletion or duplication that does not disrupt the PAM or protospacer followed by pegRNA-dependent editing of the resulting modified target sequence. Outcomes were classified as installation of additional edits from nearly matched scaffold sequence if the set of parsimonious alignments included an alignment to the pegRNA such that both the PBS and RTT were aligned to the same parts of the read as the PBS and RTT in a single alignment to the screen vector that covered the whole read and that the pegRNA alignment contained fewer edits relative to the outcome than the screen vector alignment.

### Quantification of CRISPRi-Induced Changes in Outcome Frequencies

Following categorization of all outcomes for all CRISPRi sgRNAs, counts of each category for each sgRNA are collected into a matrix for downstream analysis, and the total frequency of each category across all outcomes from cells receiving non-targeting sgRNAs is calculated to establish unperturbed baseline frequencies. Because not all CRISPRi sgRNAs achieve high levels of knockdown, calculation of gene-level effects of CRISPRi sgRNAs on outcome categories must strike a balance between assigning increased confidence to phenotypes that are supported by multiple sgRNAs per gene without penalizing genes if not all sgRNAs targeting the gene have high activity. To do this, gene-level changes in outcome category frequencies in a given screen replicate (used in FIGS. 40E-40G, FIGS. 41E-41H, FIGS. 47A-47I) are calculated by first computing the log 2 fold change in frequency of the category for every targeting sgRNA relative to the combined frequency across all non-targeting sgRNAs. For each gene, the gene-level log 2 fold change is then taken to be the mean of these values for the two sgRNAs targeting the gene with the most extreme absolute values. To provide a qualitative estimate of the range of values produced in the absence of genuine signal by this process of selecting extreme values, the sixty non-targeting sgRNAs were randomly partitioned into 20 sets of 3 quasi-genes and the same process was applied to these quasi-genes.

### Quantification of Deletion Boundaries

To maximize signal to noise in calculation of position-specific profiles of deletion frequencies and in relative fraction of deletions that removed sequence far outside of programmed nicks, outcomes from all sgRNAs targeting each gene were grouped together. In each such group of outcomes, an array of counts for every position in the screen vector was initialized to 0. For each read classified as a deletion, an interval from the first position deleted to the last position deleted was incremented by 1 in the array of counts. The final array of counts was then divided by the total number of outcomes. Deletions flanked by microhomology result in sequence outcomes that are consistent with two or more degenerate pairs of deletion boundaries. For these deletions, the pair with the minimum values in the coordinate system of the screen vector was arbitrarily chosen. To prevent primer dimers or other non-specific amplification products from being incorrectly identified as long deletions, apparent deletions for which the deleted region overlapped a window of 10-nt around either amplicon primer were excluded from calculation of deletion boundary statistics.

### Design of Recoded Sa-pegRNA Scaffold

In Repair-seq screens performed, an unintended editing outcome was observed in which additional edits are installed from nearly-matched scaffold sequence. This outcome contains a +17 T⋅A-to-C⋅G and +19 C⋅G insertion, in addition to the intended +6 G⋅C-to-C⋅G transversion. These unintended edits are consistent with incorporation of an extended 3′ DNA flap generated from reverse transcription of the Sa-pegRNA scaffold sequence into the genome. Because this extended 3′ flap shares 5-nt of homology (5′-GCCAA-3′) with the genomic target sequence after the last edited nucleotide, it was hypothesized that disrupting this homology could reduce the frequency of incorporating these unintended edits from reverse transcription of the Sa-pegRNA scaffold. A recoded Sa-pegRNA was therefore designed that alters two base pairs within the Sa-pegRNA scaffold while preserving the same base pairing interactions. The extended 3′ flap templated by this recoded Sa-pegRNA has reduced homology with the genomic target sequence. The spacer, PBS, and RT template sequences of the recoded Sa-pegRNA are identical to those for the Sa-pegRNA used in Repair-seq screens. It was observed that prime editing with this recoded Sa-pegRNA mediates similar frequencies of intended editing but substantially reduced unintended scaffold sequence incorporation compared to the original Sa-pegRNA used in Repair-seq screens.

HEK293T siRNA Transfection

For experiments in FIG. 42C, HEK293T cells were seeded on 6-well plates (Corning) at 7.5×10⁵ cells per well in DMEM plus GlutaMAX supplemented with 10% FBS. At 60% confluency 16 h after seeding, cells were transfected with 9 μL Lipofectamine RNAiMAX (Thermo Fisher Scientific) according to the manufacturer's protocol and 90 pmol ON-TARGETplus SMARTpool siRNAs (Horizon Discovery). One day after transfection, media was replaced with fresh DMEM plus GlutaMAX supplemented with 10% FBS. 2 days after transfection, cells were washed once with PBS and resuspended using TrypLE (Thermo Fisher Scientific) and DMEM plus GlutaMAX supplemented with 10% FBS. HEK293T cells were then seeded on 96-well plates (Corning) at 2.5×10⁴ cells per well. Between 16 and 24 h after seeding, cells were transfected at 60-80% confluency with 0.5 μL Lipofectamine 2000 (Thermo Fisher Scientific) according to the manufacturer's protocol and 200 ng prime editor plasmid, 66 ng pegRNA plasmid, 22 ng sgRNA plasmid (when required), and 5 pmol of the same ON-TARGETplus SMARTpool siRNAs used in the first transfection. For control conditions, cells were treated with non-targeting siRNAs in both transfections. For experiments in FIG. 49F, only the second transfection with PE components and siRNA was performed. Cells were cultured for 72 h after the second transfection before genomic DNA extraction. Real time quantitative PCR

To measure RNAi knockdown (FIG. 49E), RNA was isolated from HEK293T cells 72 h after the second siRNA transfection and converted to cDNA using the SYBR Green Fast Advanced Cells-to-CT Kit (Thermo Fisher Scientific) with cell lysis for 10-15 min using lysis solution containing 1:50 DNasel to fully digest genomic DNA. All other steps were carried out according to the manufacturer's protocol. Each 20 μL qPCR reaction was performed in technical and biological triplicate with 500 nM of each primer, 2 μL cDNA, 1×SYBR Green (Thermo Fisher Scientific), and 10 μL Q5 High-Fidelity 2× Master Mix (New England BioLabs) on a CFX96 Touch Real-Time PCR Detection System (Bio-Rad Laboratories) with the following thermocycling conditions: 98° C. for 2 min, and 40 cycles of [98° C. for 15 s, 65° C. for 20 s, and 72° C. for 30 s]. β-actin (ACTB) served as a housekeeping gene to normalize the amount of cDNA in each qPCR reaction. Relative RNA abundances from gene knockdown were calculated in comparison to a non-targeting siRNA control by the 2^{-ΔΔCT}, method. A list of primers used for qPCR reactions is provided in Table 8.

### Plasmid Transfection Dose Titration in HEK293T Cells

For experiments in FIG. 50B, HEK293T cells were seeded on 96-well plates (Corning) at 1.6-1.8×10⁴ cells per well in DMEM plus GlutaMAX supplemented with 10% FBS. Between 16 and 24 h after seeding, cells were transfected at 60-80% confluency with 0.5 μL Lipofectamine 2000 (Thermo Fisher Scientific) according to the manufacturer's protocol and 66 ng pegRNA plasmid, 0-200 ng PE2 plasmid, and 0-100 ng for MLH1dn or RFP plasmid, pUC19 filler plasmid was combined with PE2, MLH1dn, and RFP plasmids in different amounts to maintain a constant amount of total plasmid transfected (366 ng). In titrations varying the amount of total editor and in trans protein together, PE2 plasmid was used at a mass ratio of 2:1 with MLH1dn and RFP. Genomic DNA was isolated from cells 72 h after transfection.

### Generation of MLH1 Knock-Out HeLa Cell Clones

One clonal wild-type HeLa cell line and two clonal ΔMLH1 HeLa lines were used to compare prime editing enhancement from MLH1dn expression versus MLH1 knockout (FIG. 50F). To generate clonal lines, HeLa cells were seeded on 6-well plates (Corning) at 2.5×10⁵ cells per well in DMEM plus GlutaMAX supplemented with 10% FBS. At 60% confluency 18 h after seeding, cells were transfected using 7.5 μL TransIT-HeLa reagent (Mirus Bio) according to the manufacturer's protocol with 2 μg pLX_331-Cas9 (SpCas9 with blasticidin marker, Addgene #96924) and 500 ng sgRNA plasmid (spacer, 5′-GACAGTGGTGAACCGCATCG-3′ (SEQ ID NO: 367)). To make clonal wild-type HeLa cells as a control, 500 ng pUC19 plasmid was transfected instead of sgRNA plasmid. 24 h following transfection, 10 ng μL⁻¹ blasticidin (Thermo Fisher Scientific) was added to each well to select for cells transfected with Cas9.

Three days following transfection, cells were plated on 96-well plates at 1 cell per well with conditioned DMEM plus GlutaMAX supplemented with 10% FBS. Single clones were grown and expanded for 18 days. To verify that ΔMLH1 cells contain biallelic MLH1 frameshift mutations and that control cells contain the wild-type genotype, the MLH1 locus from clonal genomic DNA was sequenced on a MiSeq (Illumina) as described above. FASTQ sequencing files of MLH1 in HeLa clones are listed in Table 3. HeLa ΔMLH1 clone 1 contains MLH1 c.55_56insA and c.41_58delinsTAACTTCC alleles. HeLa ΔMLH1 clone 2 contains MLH1 c.55_56insA and c.20_66del alleles. All prime editing experiments with these clonal HeLa lines were performed as described above for HeLa cells.

### Prime Editing of Contiguous Substitutions and Additional Silent Mutations

Seven sets of prime edits that substitute 1-5 contiguous bases (35 edits total) were tested across five loci in HEK293T cells. Within each set of contiguous substitutions, all five edits altered at least one base within the seed region of the pegRNA protospacer (+1-3 nucleotides), at least one base within the PAM sequence of the pegRNA protospacer (+5 G or +6 G), or no bases within the seed region or PAM sequence at all. Because prime edits that alter the seed region or PAM sequence are more efficiently made, the design of these contiguous substitution edits controls for these confounding effects on editing efficiency, thereby enabling comparison of editing efficiency within each set.

Six sets of prime edits that program a coding change with or without additional silent mutations (27 edits total) were tested across six gene targets in HEK293T cells. Each of the six coding edits makes a transversion at one of the PAM nucleotides of the pegRNA protospacer (+5 G or +6 G). This design controls for confounding effects on editing efficiency (as explained above), allowing comparison of editing efficiency within each set. Silent mutations were designed to be close to (typically within 5-bp from) the intended coding edit to maximize interference of MMR recognition of the intended coding edit. The frequency of reads that contain the intended coding edit without indels and with or without any additional silent mutations was quantified using CRISPResso2 as described above.

### Analysis of Prime Editor Activity at Cas9 Off-Target Sites

Prime editor activity at known Cas9 off-target sites was determined by sequencing genomic DNA from HEK293T cells 3 days after transfection with plasmids encoding PE2, pegRNAs, and MLH1dn (when required) as described above. The top 4 off-target sites for each of the HEK3, EMX1, FANCF, and HEK4 spacers previously detected by CIRCLE-seq (Tsai et al., 2017) (16 sites total) were deep sequenced from genomic DNA samples as described above. To analyze off-target editing, reads were aligned to reference off-target amplicons using CRiSPResso2 (Clement et al., 2019) in standard mode with the parameters “−q 30” and “−w 10.” Off-target reads were called as leniently as possible to capture all potential reverse transcription products. For each off-target reference amplicon, the nucleotide sequence 3′ of the Cas9 nick site (prime-editable target) was compared to the 3′ DNA flap sequence encoded by pegRNA reverse transcription. Counting from the 5′ ends, the minimum sequence of the 3′ DNA flap that deviates from prime-editable target sequence was designated as an off-target marker sequence. All reference-aligned reads that contain this off-target marker sequence directly 3′ of the Cas9 nick site (including indel-containing reads) were called as off-target reads. Off-target editing efficiencies were thus quantified as a percentage of (number of off-target reads)/(number of reference-aligned reads). For some amplicons, mismatch rates at the relevant editing position were comparable to rates at other positions in the amplicon, suggesting that context-specific sequencing errors may contribute to apparent off-target prime editing and therefore this conservative approach may overestimate the true rate of pegRNA-mediated editing at off-target sites.

### Sequencing of Microsatellite Instability in Genomic DNA

Microsatellite instability was assessed in genomic DNA from HCT116 cells, monoclonal wild-type HAP1 cells, monoclonal HAP1 cells grown for 2 months (˜60 cell division) following MSH2 knockout, HeLa cells 3 days after transfection with plasmids encoding PE2-P2A-BSD, pegRNA, and MLH1dn when required. HeLa cell transfections were performed as described above. 17 mononucleotide repeats that are highly sensitive to MMR activity and are widely used to diagnosis MMR deficiency in tumors (Bacher et al., 2004; Hempelmann et al., 2015; Umar et al., 2004) were deep sequenced from genomic DNA samples. The first PCR reaction (PCR1) amplified the microsatellite sequence of interest using primers (Integrated DNA Technologies) containing Illumina forward and reverse adapters. Each 20 μL PCR1 reaction was performed with 250 nM of each primer, 0.8 μL genomic DNA, 1×SYBR Green (Thermo Fisher Scientific), and 10 μL Q5 High-Fidelity 2× Master Mix (New England BioLabs) on a CFX96 Touch Real-Time PCR Detection System (Bio-Rad Laboratories) with the following thermocycling conditions: 98° C. for 3 min, 30 cycles of [98° C. for 15 s, 62° C. for 30 s, and 72° C. for 30 s], followed by 72° C. for 3 min. All 17 PCR 1 products amplified from the same genomic DNA sample were pooled, purified with 0.8×AMPure XP beads (Beckman Coulter), and eluted in nuclease-free water. A list of primers used for PCR 1 reactions is provided in Table 8. The subsequent PCR step (PCR2) added unique i7 and i5 Illumina barcode combinations to both ends of the PCR1 DNA amplicons to enable sample demultiplexing. Each 20 μL PCR2 reaction was performed with 500 nM of each barcoding primer, 25 ng of pooled PCR1 product, 1×SYBR Green, and 10 μL Q5 High-Fidelity 2× Master Mix on a CFX96 Touch Real-time PCR Detection System with the following thermocycling conditions: 98° C. for 2 min, 8 cycles of [98° C. for 15 s, 61° C. for 20 s, and 72° C. for 30 s], followed by 72° C. for 2 min. All PCR2 products were pooled, purified with 0.8×AMPure XP beads, and eluted in nuclease-free water. DNA amplicon libraries were quantified with a Qubit 3.0 Fluorometer (Thermo Fisher Scientific), then sequenced using the MiSeq Reagent Kit v2 (Illumina) with 300 single-read cycles. A list of FASTQ sequencing files generated in these experiments is provided in Table 7.

### Quantification of Microsatellite Instability

The seventeen microsatellites analyzed all consist of long homopolymers. To quantify the observed lengths of these microsatellites in a way that is robust against the high rate of sequencing errors observed in homopolymers, each sequencing read was searched for the sequences expected to flank the homopolymers and then considered the final length of the homopolymer to be the distance between these flanking sequences. Specifically, for each locus, the longest homopolymer within the amplicon was identified, and 20-nt of the expected reference sequence on either side was recorded. Sequencing reads were demultiplexed into their loci of origin based on the first 20-nt of each read. Within reads for each locus, for each sequencing read, the first occurrences of sequences within Hamming distance 2 of the two flanking sequences were recorded. If both flanking sequences were located in the expected relative orientation within 50-nt of each, the distance between was recorded.

In Vitro Transcription of Prime Editor and MLH1dn mRNA Used in iPSC and T Cell Experiments

As described previously (Nelson et al., 2021), plasmids were cloned to encode an inactivated T7 promoter followed by a 5′ untranslated region (UTR), Kozak sequence, coding sequences of PE2 or MLH1dn, and a 3′ UTR. T7 promoter inactivation prevents potential transcription from circular plasmid template during mRNA generation. These components together were PCR amplified with Phusion U Green Multiplex Master Mix (Thermo Fisher Scientific) using primers that correct T7 promoter inactivation and append a 119-nt poly(A) tail to the 3′ UTR. The resulting PCR product was purified with the QIAquick PCR Purification Kit (Thermo Fisher Scientific) and served as a template for subsequent in vitro transcription. PE2 and MLH1dn mRNAs were transcribed from these templates using the HiScribe T7 High-Yield RNA Synthesis Kit (New England BioLabs) with co-transcriptional capping by CleanCap AG (TriLink Biotechnologies) and full replacement of UTP with N¹-Methylpseudouridine-5′-triphosphate (TriLink Biotechnologies). Transcribed mRNAs were precipitated in 2.5 M lithium chloride (Thermo Fisher Scientific), washed twice in 70% ethanol, then dissolved in nuclease-free water. The resulting PE2 and MLH1dn mRNA was quantified with a NanoDrop One UV-Vis spectrophotometer (Thermo Fisher Scientific) and was stored at −80° C.

### Electroporation of Human Patient-Derived Induced Pluripotent Stem Cells

Prior to electroporation, 24-well culture plates (Thermo Fisher Scientific) were coated with 250 μL rhLaminin-521 (Thermo Fisher Scientific) diluted 1:40 in DPBS (Thermo Fisher Scientific) per well, and incubated at 37° C. in a 5% CO₂ incubator for 2 h. For electroporation, iPS cell colonies at 70-80% confluency were washed once with DPBS and dissociated in pre-warmed Accutase (Innovative Cell Technologies) for 10 min at 37° C. in a 5% CO₂ incubator. Next, iPS cells were gently triturated, moved into a sterile 15 mL conical tube, then combined with an equal volume of DMEM/F12 (Thermo Fisher Scientific) to quench dissociation enzyme activity. Cells were pelleted at 300 g for 3 min and resuspended in StemFlex medium (Thermo Fisher Scientific) supplemented with 10 μM Y-27632 (Cayman Chemical). Cell counts and viability were determined using the Countess II FL Automated Cell Counter (Thermo Fisher Scientific). For electroporation using the NEON Transfection System 10 μL kit (Thermo Fisher Scientific), 2×10⁵ iPS cells were pelleted at 300 g for 3 min and resuspended in 9 μL NEON Buffer R. The cell solution was combined with a 3 μL mixture of 1 μg PE2 mRNA, 90 pmol synthetic pegRNA (Integrated DNA Technologies), 60 pmol synthetic sgRNA (Synthego) when required, and 0-2 μg MLH1dn mRNA in NEON Buffer R. Synthetic pegRNAs and sgRNAs were dissolved in TE buffer (10 mM Tris-HCl, pH 8.0; 0.1 mM EDTA). Mock control electroporations were performed with 3 μL NEON Buffer R without any RNA added. Directly prior to electroporation, rhLaminin-521 was aspirated and immediately replaced with 250 μL pre-warmed StemFlex medium supplemented with 10 μM Y-27632 per 24-well. Next, 10 μL of the combined cell and RNA mixture was electroporated using the NEON Transfection System (Thermo Fisher Scientific) with the following parameters: 1400 V, 20 ms, one pulse. Cells were seeded immediately into rhLaminin-521-coated 24-well plates with 250 μL StemFlex medium supplemented with 10 μM Y-27632 per well. Media was changed the following day with 500 μL StemFlex medium supplemented with 5 μM Y-27632, 72 h following electroporation, media was changed to 500 μL StemFlex medium per well. Genomic DNA was extracted 96 h after electroporation by washing iPS cells once with DPBS, lysing with gDNA lysis buffer (10 mM Tris-HCl, pH 8.0; 0.05% SDS; 800 units μL⁻¹ proteinase K (New England BioLabs)) at 37° C. for 2 h, followed by enzyme inactivation at 80° C. for 30 min. All iPSC electroporations were performed in technical duplicate and biological triplicate. Following amplicon sequencing of the edited CDKL5 locus, frequencies of intended editing and indels were quantified with CRISPResso2 in HDR mode, as described above. Because patient-derived iPSCs were heterozygous for the c.1412delA allele, frequency of editable alleles with the intended edit was quantified as: (editing frequency−editing frequency in mock controls)/(100−editing frequency in mock controls). Frequency of editable alleles with indels was quantified as described above: (total number of indel-containing reads)/(number of amplicon-aligned reads). The resulting frequencies of editable alleles with the intended edit or indels were averaged between technical duplicates, and values from biological triplicates are shown.

### Electroporation of Primary Human T Cells

Prior to electroporation, T cells were activated for 2 days with Dynabeads Human T-Expander CD3/CD28 (Thermo Fisher Scientific) and cultured at 37° C. and 5% CO₂ in T cell media (X-VIVO 15 Serum-free Hematopoietic Cell Medium (Lonza), supplemented with 5% AB human serum (Valley Biomedical), 1× GlutaMAX (Thermo Fisher Scientific), 12 mM N-acetyl-cysteine (Sigma Aldrich), 50 U mL⁻¹ penicillin and 50 μg mL⁻¹ streptomycin (Thermo Fisher Scientific), 300 IU mL⁻¹ IL-2 (Peprotech), and 5 ng mL⁻¹ recombinant human IL-7 (Peprotech) and IL-15 (Peprotech)). CD3/CD28 beads were removed from cells 5-7 h before electroporation. For electroporation using the NEON Transfection System 10 μL kit (Thermo Fisher Scientific), 3.0-3.5×10⁵ cells per sample were pelleted by centrifugation for 5 min at 300 g and resuspended in 11 μL NEON Buffer T. The cell solution was added to a mix of 1 μg PE2 mRNA, 90 pmol synthetic pegRNA (Integrated DNA Technologies), 60 pmol synthetic sgRNA (Synthego), and 0-2 μg MLH1dn mRNA. Synthetic pegRNAs and sgRNAs were dissolved in TE buffer (10 mM Tris-HCl, pH 8.0; 0.1 mM EDTA). Mock control electroporations were performed with 3 μL NEON Buffer T without any RNA added. Electroporation on the NEON Transfection System (Thermo Fisher Scientific) was carried out using 10 μL NEON tips with the following parameters: 1,400 V, 10 ms, three pulses. Cells were plated in 600 μL fresh T cell media in a 24-well plate. 2 days after electroporation, cell counts and viability were determined using the Countess II Automated Cell Counter (Thermo Fisher Scientific) and 1 mL fresh T cell media was added to cells. 4 days after electroporation, cells were pelleted by centrifugation for 5 min at 300 g and genomic DNA was isolated using the PureLink Genomic DNA Mini Kit (Thermo Fisher Scientific) following the “mammalian cells lysate” protocol with elution in nuclease-free water.

### Quantification and Statistical Analysis

The number of independent biological replicates and technical replicates for each experiment are described in the Brief Description of the Drawings or the Methods details section. In FIG. 44G, a non-parametric Mann-Whitney U test was used to compare prime editing data in HEK293T cells with prime editing data in HeLa, K562, and U2OS cells. This Example refers to Tables 5-7, which are provided below:

Lengthy table referenced here
US20250327045A1-20251023-T00001
Please refer to the end of the specification for access instructions.

Lengthy table referenced here
US20250327045A1-20251023-T00002
Please refer to the end of the specification for access instructions.

Lengthy table referenced here
US20250327045A1-20251023-T00003
Please refer to the end of the specification for access instructions.

### Example 2: Development of PEmax

To further improve prime editing, the PE2 protein was optimized by varying reverse transcriptase (RT) codon usages, the length and composition of the peptide linkers between nCas9 and the reverse transcriptase, the location, composition, and number of NLS sequences, and mutations within the SpCas9 domain (FIGS. 55A and 55B). Among 20 such variants tested, the greatest enhancement in editing efficiency was observed with a prime editor architecture that uses a Genscript human codon-optimized RT, a 34-aa linker containing a bipartite SV40 NLS (Wu et al., 2009), an additional C-terminal c-Myc NLS (Dang and Lee, 1988), and R221K and N394K mutations in SpCas9 previously shown to improve Cas9 nuclease activity (Spencer and Zhang, 2017) (FIGS. 54A and 55A). This optimized prime editor architecture was designated as PEmax. Across seven substitution edits targeting different loci, using the PEmax architecture with the PE2 system (PE2max) increased the average frequency of intended editing by 2.3-fold in HeLa cells and 1.2-fold in HEK293T cells over the original PE2 architecture (FIG. 55B). Similarly, PE3 using the PEmax architecture (PE3max) increased average editing efficiencies over PE3 by 3.2-fold in HeLa cells and 1.2-fold in HEK293T cells, without substantially changing product purity (FIGS. 54A and 55A).

### REFERENCES

Acharya, S., Wilson, T., Gradia, S., Kane, M. F., Guerrette, S., Marsischky, G. T., Kolodner, R., and Fishel, R. (1996), hMSH2 forms specific mispair-binding complexes with hMSH3 and hMSH6. Proceedings of the National Academy of Sciences 93, 13629-13634.
Amato, A., Cappabianca, M. P., Perri, M., Zaghis, I., Grisanti, P., Ponzini, D., and Di Biagio, P. (2014). Interpreting elevated fetal hemoglobin in pathology and health at the basic laboratory level: new and known γ-gene mutations associated with hereditary persistence of fetal hemoglobin. International Journal of Laboratory Hematology 36, 13-19.
Anzalone, A. V., Koblan, L. W., and Liu, D. R. (2020). Genome editing with CRISPR-Cas nucleases, base editors, transposases and prime editors. Nature Biotechnology 38, 824-844.
Anzalone, A. V., Randolph. P. B., Davis, J. R., Sousa, A. A., Koblan, L. W., Levy, J. M., Chen, P. J., Wilson, C., Newby, G. A., Raguram, A., et al. (2019). Search-and-replace genome editing without double-strand breaks or donor DNA. Nature.
Asante, E. A., Smidak, M., Grimshaw, A., Houghton, R., Tomlinson, A., Jeelani, A., Jakubcova, T., Hamdan, S., Richard-Londt, A., Linehan, J. M., et al. (2015). A naturally occurring variant of the human prion protein completely prevents prion disease. Nature 522, 478-481.
Bacher, J. W., Flanagan. L. A., Smalley, R. L., Nassif, N. A., Burgart, L. J., Halberg, R. B., Megid, W. M. A., and Thibodeau, S. N. (2004). Development of a fluorescent multiplex assay for detection of MSI-High tumors. Disease Markers 20, 237-250.
Bartlett, D. W., and Davis, M. E. (2006). Insights into the kinetics of siRNA-mediated gene silencing from live-cell and live-animal bioluminescent imaging. Nucleic Acids Research 34, 322-333.
Bosch, J. A., Birchak, G., and Perrimon, N. (2021). Precise genome engineering in Drosophila using prime editing. Proceedings of the National Academy of Sciences 118, e2021996118.
Bothmer, A., Phadke, T., Barrera, L. A., Margulies, C. M., Lee, C. S., Buquicchio, F., Moss, S., Abdulkerim, H. S., Selleck, W., Jayaram, H., et al. (2017). Characterization of the interplay between DNA repair and CRISPR/Cas9-induced DNA lesions at an endogenous locus. Nature Communications 8, 13905.
Cavaleiro, A. M., Kim, S. H., Seppals, S., Nielsen, M. T., and Norholm, M. H. H. (2015). Accurate DNA Assembly and Genome Engineering with Optimized Uracil Excision Cloning. ACS Synthetic Biology 4, 1042-1046.
Chen, P.-F., Chen, T., Forman, T. E., Swanson, A. C., O'Kelly, B., Dwyer, S. A., Buttermore, E. D., Kleiman, R., Js Carrington, S., Lavery, D. J., et al. (2021). Generation and characterization of human induced pluripotent stem cells (iPSCs) from three male and three female patients with CDKL5 Deficiency Disorder (CDD). Stem Cell Research 53, 102276.
Clement, K., Rees. H., Canver, M. C., Gehrke, J. M., Farouni, R., Hsu, J. Y., Cole, M. A., Liu, D. R., Joung, J. K., Bauer, D. E., et al. (2019). CRISPResso2 provides accurate and rapid genome editing sequence analysis. Nature Biotechnology 37, 224-226.
Dang, C. V., and Lee, W. M. (1988). Identification of the human c-myc protein nuclear translocation signal. Molecular and Cellular Biology 8, 4048-4054.
Engler, C., Kandzia, R., and Marillonnet, S. (2008). A One Pot, One Step, Precision Cloning Method with High Throughput Capability. PLoS ONE 3, e3647.
Fang, W. H., and Modrich, P. (1993). Human strand-specific mismatch repair occurs by a bidirectional mechanism similar to that of the bacterial reaction. Journal of Biological Chemistry 268, 11838-11844.
Fishel, R., Lescoe, M. K., Rao, M. R. S., Copeland, N. G., Jenkins, N. A., Garber, J., Kane, M., and Kolodner, R. (1993). The human mutator gene homolog MSH2 and its association with hereditary nonpolyposis colon cancer. Cell 75, 1027-1038.
Geng, H., Du, C., Chen, S., Salerno, V., Manfredi, C., and Hsieh, P. (2011). In vitro studies of DNA mismatch repair proteins. Analytical Biochemistry 413, 179-184.
Genschel, J., Bazemore, L. R., and Modrich, P. (2002). Human Exonuclease I Is Required for 5′ and 3′ Mismatch Repair. Journal of Biological Chemistry 277, 13302-13311.
Genschel, J., Littman, S. J., Drummond, J. T., and Modrich, P. (1998). Isolation of MutSβ from Human Cells and Comparison of the Mismatch Repair Specificities of MutSβ and MutSα. Journal of Biological Chemistry 273, 19895-19901.
Gilbert, Luke A., Horlbeck, Max A., Adamson, B., Villalta, Jacqueline E., Chen, Y., Whitehead, Evan H., Guimaraes, C., Panning, B., Ploegh, Hidde L., Bassik, Michael C., et al. (2014). Genome-Scale CRISPR-Mediated Control of Gene Repression and Activation. Cell 159, 647-661.
Gilbert, Luke A., Larson, Matthew H., Morsut, L., Liu. Z., Brar, Gloria A., Torres. Sandra E., Stern-Ginossar, N., Brandman, O., Whitehead, Evan H., Doudna, Jennifer A., et al. (2013). CRISPR-Mediated Modular RNA-Guided Regulation of Transcription in Eukaryotes. Cell 154, 442-451.
Gueneau. E., Dherin. C., Legrand, P., Tellier-Lebegue, C., Gilquin, B., Bonnesoeur, P., Londino, F., Quemener, C., Le Du, M.-H., Mdrquez, J. A., et al. (2013). Structure of the MutLα C-terminal domain reveals how Mlh1 contributes to Pms1 endonuclease site. Nature Structural & Molecular Biology 20, 461-468.
Guerrette, S., Acharya, S., and Fishel, R. (1999). The Interaction of the Human MutL Homologues in Hereditary Nonpolyposis Colon Cancer. Journal of Biological Chemistry 274, 6336-6341.
Gupta, S., Gellert, M., and Yang, W. (2012). Mechanism of mismatch recognition revealed by human MutSβ bound to unpaired DNA loops. Nature Structural & Molecular Biology 19, 72-78.
Hempelmann, J. A., Scroggins, S. M., Pritchard, C. C., and Salipante, S. J. (2015). MSIplus for Integrated Colorectal Cancer Molecular Testing by Next-Generation Sequencing. The Journal of Molecular Diagnostics 17, 705-714.
Hussmann, J. A., Ling, J., Ravisankar. P., Yan, J., Cirincione, A., Xu, A., Simpson, D., Yang, D., Bothmer, A., Cotta-Ramusino, C., et al. (2021). Repair-seq enables systematic mapping of DNA repair processes in genome editing. Submitted.
Iaccarino. I., Marra, G., and Palomba, F. J., J. (1998), hMSH2 and hMSH6 play distinct roles in mismatch binding and contribute differently to the ATPase activity of hMutSalpha. The EMBO Journal 17, 2677-2686.
Ingram, V. M. (1956). A Specific Chemical Difference Between the Globins of Normal Human and Sickle-Cell Anaemia Hmmoglobin. Nature 178, 792-794.
Iyer, R. R., Pluciennik, A., Burdett, V., and Modrich, P. L. (2006). DNA Mismatch Repair: Functions and Mechanisms. Chemical Reviews 106, 302-323.
Jin, S., Lin, Q., Luo, Y., Zhu, Z., Liu, G., Li, Y., Chen, K., Qiu, J.-L., and Gao, C. (2021). Genome-wide specificity of prime editors in plants. Nature Biotechnology.
Kadyrov, F. A., Dzantiev, L., Constantin, N., and Modrich, P. (2006). Endonucleolytic Function of MutLα in Human Mismatch Repair. Cell 126, 297-308.
Kim, D. Y., Moon, S. B., Ko, J.-H., Kim. Y.-S., and Kim, D. (2020). Unbiased investigation of specificities of prime editing systems in human cells. Nucleic Acids Research 48, 10576-10589.
Kim, J. H., Lee, S.-R., Li, L.-H., Park, H.-J., Park, J.-H., Lee, K. Y., Kim, M.-K., Shin, B. A., and Choi, S.-Y. (2011). High Cleavage Efficiency of a 2A Peptide Derived from Porcine Teschovirus-1 in Human Cell Lines, Zebrafish and Mice. PLoS ONE 6, e18556.
Kunkel, T. A., and Erie, D. A. (2005). DNA mismatch repair. Annu Rev Biochem 74, 681-710.
Lahue, R. S., Au, K. G., and Modrich. P. (1989). DNA mismatch correction in a defined system. Science 245, 160.
Leach. F. S., Nicolaides. N. C., Papadopoulos, N., Liu, B., Jen, J., Parsons, R., Peltomaki, P., Sistonen, P., Aaltonen, L. A., Nystrom-Lahti, M., et al. (1993). Mutations of a mutS homolog in hereditary nonpolyposis colorectal cancer. Cell 75, 1215-1225.
Li, G.-M. (2008). Mechanisms and functions of DNA mismatch repair. Cell Research 18, 85-98.
Lin, Q., Zong. Y., Xue, C., Wang, S., Jin, S., Zhu, Z., Wang, Y., Anzalone, A. V., Raguram, A., Doman, J. L., et al. (2020). Prime genome editing in rice and wheat. Nature Biotechnology 38, 582-585.
Liu, P., Liang, S.-Q., Zheng, C., Mintzer, E., Zhao, Y. G., Ponnienselvan, K., Mir, A., Sontheimer, E. J., Gao, G., Flotte, T. R., et al. (2021). Improved prime editors enable pathogenic allele correction and cancer modelling in adult mice. Nature Communications 12.
Liu, S., Wang, Q., Yu, X., Li, Y., Guo, Y., Liu, Z., Sun, F., Hou, W., Li, C., Wu, L., et al. (2018). HIV-1 inhibition in cells with CXCR4 mutant genome created by CRISPR-Cas9 and piggyBac recombinant technologies. Scientific Reports 8.
Liu, Y., Li, X., He. S., Huang. S., Li, C., Chen. Y., Liu. Z., Huang, X., and Wang. X. (2020). Efficient generation of mouse models with the prime editing system. Cell Discovery 6.
Lujan, S. A., Clausen, A. R., Clark, A. B., MacAlpine, H. K., MacAlpine. D. M., Malc, E. P., Mieczkowski, P. A., Burkholder, A. B., Fargo, D. C., Gordenin, D. A., et al. (2014). Heterogeneous polymerase fidelity and mismatch repair bias genome variation and composition. Genome Res 24, 1751-1764.
Mead, S., Whitfield, J., Poulter, M., Shah, P., Uphill, J., Campbell, T., Al-Dujaily, H., Hummerich, H., Beck, J., Mein. C. A., et al. (2009). A Novel Protective Prion Protein Variant that Colocalizes with Kuru Exposure. New England Journal of Medicine 361, 2056-2065.
Nelson, J. W., Randolph, P. B., Shen. S. P., Everette, K. A., Chen, P. J., Anzalone. A. V., Newby, G. A., An, M., Chen, J. C., and Liu, D. R. (2021). Engineered pegRNAs that improve prime editing efficiency. Submitted.
Olson, H. E., Demarest, S. T., Pestana-Knight, E. M., Swanson, L. C., Iqbal, S., Lal, D., Leonard, H., Cross, J. H., Devinsky, O., and Benke, T. A. (2019). Cyclin-Dependent Kinase-Like 5 Deficiency Disorder: Clinical Review. Pediatric Neurology 97, 18-25.
Parsons, R., Li, G.-M., Longley, M. J., Fang, W.-H., Papadopoulos, N., Jen. J., De La Chapelle, A., Kinzler, K. W., Vogelstein, B., and Modrich, P. (1993). Hypermutability and mismatch repair deficiency in RER+ tumor cells. Cell 75, 1227-1236.
Petri, K., Zhang, W., Ma, J., Schmidts, A., Lee, H., Horng, J. E., Kim, D. Y., Kurt, I. C., Clement, K., Hsu, J. Y., et al. (2021). CRISPR prime editing with ribonucleoprotein complexes in zebrafish and primary human cells. Nature Biotechnology.
Plotz, G., Raedle, J., Brieger, A., Trojan, J., and Zeuzem, S. (2003). N-terminus of hMLH1 confers interaction of hMutL and hMutL with hMutS. Nucleic Acids Research 31, 3217-3226.
Pluciennik. A., Dzantiev, L., Iyer, R. R., Constantin, N., Kadyrov, F. A., and Modrich. P. (2010). PCNA function in the activation and strand direction of MutL endonuclease in mismatch repair. Proceedings of the National Academy of Sciences 107, 16066-16071.
Ran, F. A., Cong, L., Yan, W. X., Scott, D. A., Gootenberg, J. S., Kriz, A. J., Zetsche, B., Shalem, O., Wu, X., Makarova, K. S., et al. (2015). In vivo genome editing using Staphylococcus aureus Cas9. Nature 520, 186-191.
Räschle, M., Dufner, P., Marra, G., and Jiricny, J. (2002). Mutations within the hMLH1 and hPMS2 Subunits of the Human MutLα Mismatch Repair Factor Affect Its ATPase Activity, but Not Its Ability to Interact with hMutSα. Journal of Biological Chemistry 277, 21810-21820.
Schene, I. F., Joore, I. P., Oka. R., Mokry. M., Van Vugt. A. H. M., Van Boxtel, R., Van Der Doef, H. P. J., Van Der Laan, L. J. W., Verstegen, M. M. A., Van Hasselt, P. M., et al. (2020). Prime editing for functional repair in patient-derived disease models. Nature Communications 11.
Shcherbakova, P. V., and Kunkel, T. A. (1999). Mutator phenotypes conferred by MLH1 overexpression and by heterozygosity for mlh1 mutations. Molecular and cellular biology 19, 3177-3183.
Sockolosky. J. T., Trotta, E., Parisi, G., Picton. L., Su, L. L., Le, A. C., Chhabra. A., Silveria, S. L., George, B. M., King, I. C., et al. (2018). Selective targeting of engineered T cells using orthogonal IL-2 cytokine-receptor complexes. Science 359, 1037-1042.
Spencer, J. M., and Zhang, X. (2017). Deep mutational scanning of S. pyogenes Cas9 reveals important functional domains. Scientific Reports 7.
Strand, M., Prolla. T. A., Liskay, R. M., and Petes, T. D. (1993). Destabilization of tracts of simple repetitive DNA in yeast by mutations affecting DNA mismatch repair. Nature 365, 274-276.
Su, S. S., Lahue, R. S., Au, K. G., and Modrich, P. (1988). Mispair specificity of methyl-directed DNA mismatch correction in vitro. Journal of Biological Chemistry 263, 6829-6835.
Sugawara, N., Goldfarb, T., Studamire, B., Alani, E., and Haber, J. E. (2004). Heteroduplex rejection during single-strand annealing requires Sgs1 helicase and mismatch repair proteins Msh2 and Msh6 but not Pms1. Proceedings of the National Academy of Sciences 101, 9315-9320.
Supek. F., and Lehner, B. (2015). Differential DNA mismatch repair underlies mutation rate variation across the human genome. Nature 521, 81-84.
Sürün, D., Schneider. A., Mircetic, J., Neumann. K., Lansing, F., Paszkowski-Rogacz. M., Hänchen, V., Lee-Kirsch. M. A., and Buchholz. F. (2020). Efficient Generation and Correction of Mutations in Human iPS Cells Utilizing mRNAs of CRISPR Base Editors and Prime Editors. Genes 11, 511.
Thomas. D. C., Roberts, J. D., and Kunkel, T. A. (1991). Heteroduplex repair in extracts of human HeLa cells. Journal of Biological Chemistry 266, 3744-3751.
Tomer, G., Buermeyer, A. B., Nguyen, M. M., and Liskay, R. M. (2002). Contribution of human mlh1 and pms2 ATPase activities to DNA mismatch repair. J Biol Chem 277, 21801-21809.
Tran, H. T., Keen, J. D., Kricker, M., Resnick, M. A., and Gordenin, D. A. (1997). Hypermutability of homonucleotide runs in mismatch repair and DNA polymerase proofreading yeast mutants. Molecular and Cellular Biology 17, 2859-2865.
Trojan. J., Zeuzem, S., Randolph. A., Hemmerle, C., Brieger, A., Raedle, J., Plotz, G., Jiricny, J., and Marra, G. (2002). Functional analysis of hMLH1 variants and HNPCC-related mutations using a human expression system. Gastroenterology 122, 211-219.
Tsai, S. Q., Nguyen, N. T., Malagon-Lopez, J., Topkar, V. V., Aryee, M. J., and Joung, J. K. (2017). CIRCLE-seq: a highly sensitive in vitro screen for genome-wide CRISPR-Cas9 nuclease off-targets. Nature Methods 14, 607-614.
Umar, A., Boland, C. R., Terdiman, J. P., Syngal, S., Chapelle, A. D. L., Ruschoff, J., Fishel, R., Lindor, N. M., Burgart, L. J., Hamelin. R., et al. (2004). Revised Bethesda Guidelines for Hereditary Nonpolyposis Colorectal Cancer (Lynch Syndrome) and Microsatellite Instability. JNCI Journal of the National Cancer Institute 96, 261-268.
Umar. A., Boyer, J. C., and Kunkel, T. A. (1994). DNA loop repair by human cell extracts. Science 266, 814.
Warren, J. J., Pohlhaus, T. J., Changela, A., Iyer, R. R., Modrich, P. L., and Beese, Lorena S. (2007). Structure of the Human MutSα DNA Lesion Recognition Complex. Molecular Cell 26, 579-592.
Wu, J., Corbett, A. H., and Berland, K. M. (2009). The Intracellular Mobility of Nuclear Import Receptors and NLS Cargoes. Biophysical Journal 96, 3840-3849.
Zhang, Y., Yuan. F., Presnell, S. R., Tian. K., Gao, Y., Tomkinson, A. E., Gu, L., and Li, G.-M. (2005). Reconstitution of 5′-Directed Human Mismatch Repair in a Purified System. Cell 122, 693-705.
Zhou, B. P., Liao, Y., Xia, W., Spohn, B., Lee, M.-H., and Hung, M.-C. (2001). Cytoplasmic localization of p21Cip1/WAFI by Akt-induced phosphorylation in HER-2/neu-overexpressing cells. Nature Cell Biology 3, 245-252.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following embodiments.

LENGTHY TABLES
The patent application contains a lengthy table section. A copy of the table is available in electronic form from the USPTO web site (https://seqdata.uspto.gov/docdetail?docId=US20250327045A1). An electronic copy of the table will also be available from the USPTO upon request and payment of the fee set forth in 37 CFR 1.19(b)(3).

1-191. (canceled)

192. A prime editor comprising (i) a nucleic acid programmable DNA binding protein (napDNAbp) and (ii) a DNA polymerase, wherein the napDNAbp is a Cas9 nickase (nCas9) comprising a R221K amino acid substitution, a N394K amino acid substitution, and an amino acid substitution that inactivates HNH domain nuclease activity, or corresponding amino acid substitutions, relative to a wild type Cas9 as set forth in SEQ ID NO: 2.

193-329. (canceled)

330. The prime editor of claim 192, wherein the nCas9 is connected to the DNA polymerase in a fusion protein.

331. The prime editor of claim 330, wherein the fusion protein comprises the structure: NH₂-[napDNAbp]-[DNA polymerase]-COOH, wherein “]-[” indicates an optional linker sequence.

332. The prime editor of claim 192, wherein the nCas9 comprises a R221K, a N394K, and a H840X amino acid substitution compared to a wild type Cas9 as set forth in SEQ ID NO: 2, wherein X is any amino acid other than histidine.

333. The prime editor of claim 332, wherein the nCas9 comprises a H840A amino acid substitution compared to a wild type Cas9 as set forth in SEQ ID NO: 2.

334. The prime editor of claim 192, wherein the nCas9 comprises an amino acid sequence having at least 80% sequence identity with SEQ ID NO: 104.

335. The prime editor of claim 192, wherein the nCas9 comprises the amino acid sequence of SEQ ID NO: 104.

336. The prime editor of claim 330, wherein the nCas9 and the DNA polymerase are connected by a linker in the fusion protein.

337. The prime editor of claim 336, wherein the linker comprises the sequence of SEQ ID NO: 105.

338. The prime editor of claim 192, wherein the prime editor further comprises (i) a SV40 NLS at the N terminus, and (ii) a SV40 NLS and/or a c-Myc NLS at the C terminus.

339. The prime editor of claim 338, wherein the N terminus SV40 NLS comprises SEQ ID NO: 101, the C terminus SV40 NLS comprises SEQ ID NO: 140, and the C terminus c-Myc NLS comprises SEQ ID NO: 135.

340. The prime editor of claim 192, wherein the DNA polymerase is a reverse transcriptase.

341. The prime editor of claim 340, wherein the reverse transcriptase is a retroviral reverse transcriptase.

342. The prime editor of claim 340, wherein the reverse transcriptase is a Moloney-Murine Leukemia Virus reverse transcriptase (MMLV-RT) comprising an amino acid sequence having at least 80% sequence identity with any one of SEQ ID NOs: 81-98.

343. The prime editor of claim 340, wherein the reverse transcriptase is a Moloney-Murine Leukemia Virus reverse transcriptase (MMLV-RT) comprising an amino acid sequence of any one of SEQ ID NOs: 81-98.

344. The prime editor of claim 340, wherein the reverse transcriptase comprises a D200N, a T330P, a T306K, a W313F, and a L603W amino acid substitution relative to MMLV-RT as set forth in SEQ ID NO: 81.

345. The prime editor of claim 330, wherein the fusion protein comprises an amino acid sequence having at least 80% sequence identity with SEQ ID NO: 99.

346. The prime editor of claim 330, wherein the fusion protein comprises the amino acid sequence of SEQ ID NO: 99.

347. A prime editor system comprising the prime editor of claim 192 and a prime editing guide RNA (pegRNA).

348. The prime editor system of claim 347 further comprising a second strand nicking single guide RNA (sgRNA).

349. One or more polynucleotides encoding the prime editor of claim 192.

350. A polynucleotide encoding the prime editor of claim 345.

351. A method of editing a nucleic acid molecule comprising contacting the nucleic acid molecule with the prime editor system of claim 347.
